(19) United States
(12) Patent Application Publication
(10) Pub. No.: US 2024/0245787 A1
(43) Pub. Date: Jul. 25, 2024

(54) E3 LIGASE BINDERS AND USES THEREOF

(71) Applicant: President and Fellows of Harvard College, Cambridge, MA (US)

(72) Inventors: Christina M. Woo, Cambridge, MA (US); Saki Ichikawa, Cambridge, MA (US); Hope Airlie Flaxman, Cambridge, MA (US); Wengqing Xu, Cambridge, MA (US)

(73) Assignee: President and Fellows of Harvard College, Cambridge, MA (US)

(21) Appl. No.: 18/286,740

(22) PCT Filed: Apr. 13, 2022
(86) PCT No.: PCT/US2022/024624

§ 371 Date: Oct. 12, 2023

Related U.S. Application Data
(60) Provisional application No. 63/174,389, filed on Apr. 13, 2021.
(60) Provisional application No. 63/303,364, filed on Jan. 26, 2022.

(51) Int. Cl. A61K 47/55; A61K 47/54

(52) U.S. Cl. CPC A61K 47/55; A61K 47/542

(57) ABSTRACT

The present invention relates to compounds of Formula (I′) and Formula (I), and pharmaceutically acceptable salts or tautomers thereof. Also disclosed are compositions, combination therapies, kits, uses, and methods. Exemplary uses include treating diseases and disorders including cancers (e.g., hemopoietic cancers (e.g., leukemia, lymphoma, multiple myeloma)), inflammatory diseases (e.g., erythema nodosum leprosum, arthritis, Crohn's disease, colitis, inflammatory bowel disease), and autoimmune diseases (e.g., pulmonary fibrosis, systemic lupus erythematosus).

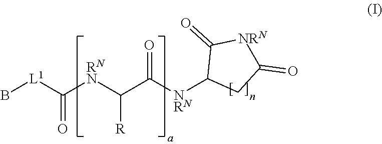

### RELATED APPLICATIONS

This application claims priority under 35 U.S.C. § 119(e) to U.S. Provisional Applications, U.S. Ser. No. 63/174,389, filed Apr. 13, 2021, and U.S. Ser. No. 63/303,364, filed Jan. 26, 2022, each of which is incorporated herein by reference.

### GOVERNMENT SUPPORT

This invention was made with government support under 1745303 awarded by the National Science Foundation (NSF). The government has certain rights in this invention.

### BACKGROUND

Cereblon (CRBN) is a conserved protein that functions as a substrate recognition adaptor in the CRL4^{CRBN} E3 ubiquitin ligase complex. The immunomodulatory drugs (IMiDs) thalidomide, lenalidomide, and pomalidomide are therapeutic agents that bind cereblon (CRBN) and modulate selection of protein substrates for ubiquitylation and degradation.¹ Degradation of these chemically-induced substrates, which include IKZF1, IKZF3, CK1a, GSPT1, SALL4, and p63, partially underlies the pleiotropic effects of the IMiDs, including their therapeutic efficacy in multiple myeloma,^{2,3} del(5q) myelodysplastic syndrome,⁴ and other hemopoietic cancers and/or malignancies,⁵ as well as the teratogenic effects observed during development.⁶⁻⁸ However, despite increasing engagement of CRBN for targeted protein degradation strategies, endogenous substrate selection mechanisms of CRBN have remained elusive. The conservation of CRBN across species and association with neurological development implies its participation in a vital biological mechanism that may be impacted during therapy.

E3 ubiquitin ligase complexes select proteins for degradation through the recognition of degrons, specific amino acid that are sufficient to promote ubiquitylation and degradation when embedded in a substrate. Short sequences at the protein N-terminus were the first discovered degrons⁹ and, more recently, several E3 ligases that recognize C-terminal degrons have been reported.^{10,11} Several degrons are generated by post-translational modifications (PTMs), such as the recognition of proline oxidation by the E3 ligase VHL.¹² Additionally, small molecules that induce degrons exist in nature (e.g., the plant hormone auxin), which are reminiscent of the activity of the IMiDs.¹³ The IMiDs may therefore chemically mimic the endogenous recognition element of the thalidomide binding domain of CRBN by either chemical-induction of a degron or represent the degron itself (FIG. 1A).

Efforts to identify a degron for the thalidomide binding domain of CRBN have sought to either discover substrates that compete for IMiD binding or reveal ligands by an in vitro structure-focused approach. Substrates for the thalidomide-binding domain of CRBN that compete for binding with thalidomide include MEIS2¹⁴ and amyloid precursor protein,¹⁵ yet a defined and transferrable degron within these substrates has not been identified. Structure-focused approaches capitalize on the finding that thalidomide binds to CRBN by creating three contacts through the glutarimide ring,^{14,16} which suggests that the degron recognized by CRBN likely possesses the potential for a similar tripartite binding pattern. Based on structural similarity to thalidomide, the degron could derive from nucleotide or amino acid sources (FIG. 1B). Mimicry of thalidomide with uridine derivatives is intriguing given the binding pattern and homology of CRBN with the RNA binding protein RIG-1¹⁴ and efforts to evaluate biological ligands for CRBN have led to the discovery of several uridine derivatives as ligands of the thalidomide binding domain of CRBN in vitro,^{17,18} although no connection of these ligands to cellular activity has been reported.

### SUMMARY OF THE INVENTION

Thalidomide and lenalidomide are proposed to mimic a naturally occurring degron; however, the structural motif recognized by the thalidomide binding domain of CRBN is unknown. Based on structural similarity to thalidomide, the degron recognized by CRBN may consist of a C-terminal cyclic imide, such that thalidomide may mimic post-translational modifications (PTMs) like pyroglutamate or cyclic imides that arise from cyclized glutamine (cQ) or cyclized asparagine (CN). These PTMs are generated in the proteome by enzymes,¹⁹ spontaneous cyclization during protein aging,²⁰⁻²³ or during specific protein splicing events (e.g., intein excision).^{24, 25} After formation, cyclic imides are presumed to undergo further hydrolysis despite their stability to mass spectrometry analysis,²⁶⁻²⁸ and may therefore be overlooked PTMs in the cellular proteome if proteins bearing these modifications are indeed recognized and degraded by CRBN.

Provided herein are C-terminal cyclic imides, post-translational modifications that arise from intramolecular cyclization of glutamine or asparagine residues, that are degrons for CRBN. Dipeptides bearing the cyclic imide degron are substitutes for thalidomide when embedded within bifunctional small molecule degraders. Thus, these ligands of CRBN act as functional and transferrable substitutes for IMiDs in cells using a targeted protein degradation strategy. Installation of the degron at the C-termini of proteins induces CRBN-dependent ubiquitylation and degradation in vitro and in cells. The discovery of the cyclic imide degron defines a novel regulatory process controlled by these modifications, which may impact the clinical development of therapeutic agents that engage CRBN.

In one aspect, provided herein are compounds of Formula (I′):

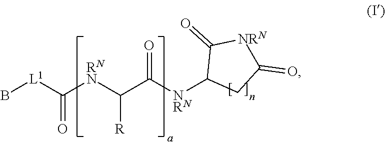

or a pharmaceutically acceptable salt or tautomer thereof, wherein B, L¹, R^{N}, R, c, and n are defined herein. In some embodiments, R is hydrogen, optionally substituted alkyl, optionally substituted carbocyclyl, optionally substituted aryl, optionally substituted heteroaryl, or -L¹-B; optionally where R and R^{N} are joined together to form a optionally substituted 6-membered ring or optionally substituted 5-membered ring; provided that only one instance of B is a binder of a target. In some embodiments, B is a binder of a target, wherein the target is a protein (e.g., a receptor, enzyme, antibody, hormone, contractile protein, hormonal protein, structural protein, storage protein, transport protein, regulatory proteins, defensive protein), polypeptide, peptide, carbohydrate, or small molecule. In some embodiments, B is a binder of a target, wherein the target is selected from the group consisting of a bromodomain, a bromodomain-containing protein, a histone methyltransferase, a kinase, a phosphorylase, a cytosolic signaling protein, a nuclear protein, a histone deacetylase, a lysine methyltransferase, a protein regulating angiogenesis, a protein regulating immune response, an aryl hydrocarbon receptor, a hormone receptor, and a transcription factor; and L¹ is a linker.

In one aspect, provided herein are compounds of Formula (I′) or (I):

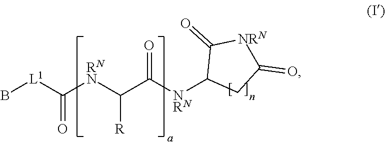

or a pharmaceutically acceptable salt or tautomer thereof, wherein B, L¹, R^{N}, R, c, and n are defined herein. In some embodiments, R is an amino acid side chain (e.g., the side chain of tyrosine, phenylalanine). In some embodiments, B is a binder of a target, wherein the target is a protein (e.g., a receptor, enzyme, antibody, hormone, contractile protein, hormonal protein, structural protein, storage protein, transport protein, regulatory proteins, defensive protein), polypeptide, peptide, carbohydrate, or small molecule. In some embodiments, B is a binder of a target wherein the target is selected from the group consisting of a bromodomain, a bromodomain-containing protein, a histone methyltransferase, a kinase, a cytosolic signaling protein, a nuclear protein, a histone deacetylase, a lysine methyltransferase, a protein regulating angiogenesis, a protein regulating immune response, an aryl hydrocarbon receptor, a hormone receptor, and a transcription factor; and L¹ is a linker.

In some embodiments, a compound of Formula (I′) or (I) is of the formula:

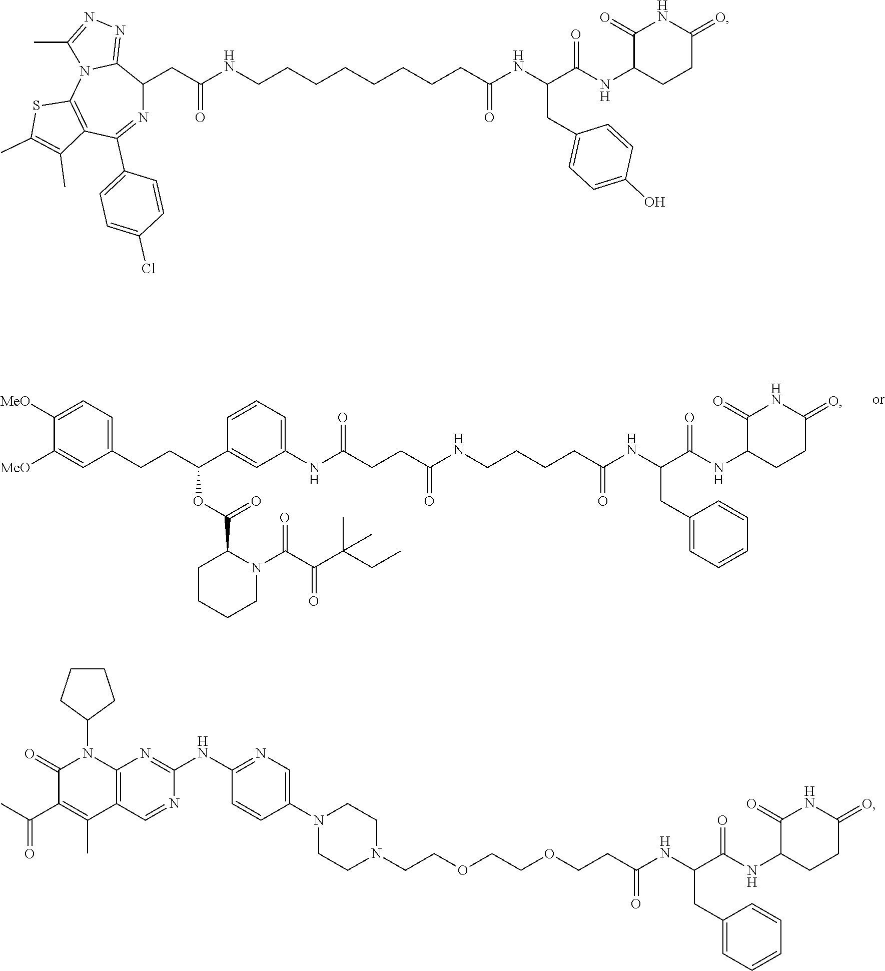 [SMILES: Cc1sc2c(c1C)C(c1ccc(Cl)cc1)=NC(CC(=O)NCCCCCCCCC(=O)NC(Cc1ccc(O)cc1)C(=O)NC1CCC(=O)NC1=O)c1nnc(C)n1-2 ; CCC(C)(C)C(=O)C(=O)N1CCCC[C@H]1C(=O)O[C@H](CCc1ccc(OC)c(OC)c1)c1cccc(NC(=O)CCC(=O)NCCCCC(=O)NC(Cc2ccccc2)C(=O)NC2CCC(=O)NC2=O)c1 ; "or" ; CC(=O)c1c(C)c2cnc(Nc3ccc(N4CCN(CCOCCOCCC(=O)NC(Cc5ccccc5)C(=O)NC5CCC(=O)NC5=O)CC4)cn3)nc2n(C2CCCC2)c1=O]

or a pharmaceutically acceptable salt or tautomer thereof.

In some embodiments, a compound of Formula (I′) or (I) is of the formula:

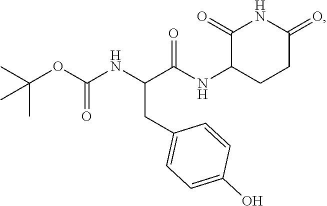 [SMILES: CC(C)(C)OC(=O)NC(Cc1ccc(O)cc1)C(=O)NC1CCC(=O)NC1=O]

or a pharmaceutically acceptable salt or tautomer thereof.

In another aspect, provided herein is a composition comprising a compound disclosed herein (e.g., a compound of Formula (I′) or (I)), or a pharmaceutically acceptable salt or tautomer thereof, and optionally a pharmaceutically acceptable excipient.

In one aspect, provided herein is a method of treating or preventing a disease in a subject, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease or disorder is an inflammatory disease, proliferative disease, autoimmune disease, hematological disease, genetic disease, neurological disease, painful condition, metabolic disorder, infectious disease, cardiovascular disease, cerebrovascular disease, tissue repair disorder, pulmonary disease, dermatological disease, bone disease, or hormonal disease. In some embodiments, the disease is cancer. In some embodiments the disease is a proliferative disease, an inflammatory disease, or an autoimmune disease. In some embodiments, the disease is associated with or mediated by bromodomain, cyclin dependent kinase, or FKBP activity. In some embodiments, the disease is associated with or mediated by bromodomain or FKBP activity.

In another aspect, provided herein is a method of treating a disease associated with a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments the disease is a proliferative disease, an inflammatory disease, or an autoimmune disease.

In a further aspect, provided herein is a method of treating a disease associated with or mediated by a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments the disease is a proliferative disease, an inflammatory disease, or an autoimmune disease.

In one aspect, provided herein is a method of treating a disease associated with aberrant (e.g., increased) activity of a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments the disease is a proliferative disease, an inflammatory disease, or an autoimmune disease.

In another aspect, provided herein is a method of modulating (e.g., inhibiting) the activity of a target (i.e., the target that B binds to) in a subject, the method comprising administering to the subject an effective amount of a compound disclosed herein (e.g., a compound of Formula (I′) or (I)), or a pharmaceutically acceptable salt or tautomer thereof, or composition disclosed herein.

In a further aspect, provided herein is a method of modulating (e.g., inhibiting) the activity of a target (i.e., the target that B binds to) in a biological sample, the method comprising contacting the biological sample with an effective amount of a compound disclosed herein (e.g., a compound of Formula (I′) or (I)), or a pharmaceutically acceptable salt or tautomer thereof, or composition disclosed herein.

In one aspect, provided herein is a method of modulating (e.g., inhibiting) the expression of a gene that is regulated by a target (i.e., the target that B binds to) in a subject, the method comprising administering to the subject an effective amount of a compound disclosed herein (e.g., a compound of Formula (I′) or (I)), or a pharmaceutically acceptable salt or tautomer thereof, or composition disclosed herein.

In another aspect, provided herein is a method of treating a disease associated with a bromodomain-containing protein, a bromodomain, a cyclin dependent kinase, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments the disease is a proliferative disease, an inflammatory disease, or an autoimmune disease. In some embodiments, the cyclin dependent kinase is CDK4. In some embodiments, the cyclin dependent kinase is CDK6. In some embodiments, the bromodomain-containing protein is BRD4. In some embodiments, the bromodomain is BRD4. In some embodiments, the FKBP is FKBP12.

In a further aspect, provided herein is a method of treating a disease associated with or mediated by a bromodomain-containing protein, a bromodomain, a cyclin dependent kinase, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments, the disease is a proliferative disease, an inflammatory disease, or an autoimmune disease. In some embodiments, the cyclin dependent kinase is CDK4. In some embodiments the cyclin dependent kinase is CDK6. In some embodiments, the bromodomain-containing protein is BRD4. In some embodiments, the bromodomain is BRD4. In some embodiments, the FKBP is FKBP12.

In one aspect, provided herein is a method of treating a disease associated with aberrant (e.g., increased) activity a bromodomain-containing protein, a bromodomain, a cyclin dependent kinase, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments the disease is a proliferative disease, an inflammatory disease, or an autoimmune disease. In some embodiments, the cyclin dependent kinase is CDK4. In some embodiments, the cyclin dependent kinase is CDK6. In some embodiments, the bromodomain-containing protein is BRD4. In some embodiments, the bromodomain is BRD4. In some embodiments, the FKBP is FKBP12.

In another aspect, provided herein is a method of modulating (e.g., inhibiting) the activity of a bromodomain-containing protein, a bromodomain, a cyclin dependent kinase, or a FKBP in a subject, the method comprising administering to the subject an effective amount of a compound disclosed herein (e.g., a compound of Formula (I′) or (I)), or a pharmaceutically acceptable salt or tautomer thereof, or composition disclosed herein. In some embodiments, the cyclin dependent kinase is CDK4. In some embodiments, the cyclin dependent kinase is CDK6. In some embodiments, the bromodomain-containing protein is BRD4. In some embodiments, the bromodomain is BRD4. In some embodiments, the FKBP is FKBP12.

In a further aspect, provided herein is a method of modulating (e.g., inhibiting) the activity of a bromodomain-containing protein, a bromodomain, a cyclin dependent kinase, or a FKBP in a biological sample, the method comprising contacting the biological sample with an effective amount of a compound disclosed herein (e.g., a compound of Formula (I′) or (I)), or a pharmaceutically acceptable salt or tautomer thereof, or composition disclosed herein. In some embodiments the cyclin dependent kinase is CDK4. In some embodiments the cyclin dependent kinase is CDK6. In some embodiments, the bromodomain-containing protein is BRD4. In some embodiments, the bromodomain is BRD4. In some embodiments, the FKBP is FKBP12.

In one aspect, provided herein is a method of modulating (e.g., inhibiting) the expression of a gene that is regulated by a bromodomain-containing protein, a bromodomain, a cyclin dependent kinase, or a FKBP in a subject, the method comprising administering to the subject an effective amount of a compound disclosed herein (e.g., a compound of Formula (I′) or (I)), or a pharmaceutically acceptable salt or tautomer thereof, or composition disclosed herein. In some embodiments the cyclin dependent kinase is CDK4. In some embodiments the cyclin dependent kinase is CDK6. In some embodiments, the bromodomain-containing protein is BRD4. In some embodiments, the bromodomain is BRD4. In some embodiments, the FKBP is FKBP12.

In another aspect, provided herein is a method of inducing the degradation of a protein in a subject, the method comprising administering to the subject an effective amount of a compound disclosed herein (e.g., a compound of Formula (I′) or (I)), or a pharmaceutically acceptable salt or tautomer thereof, or composition disclosed herein.

In a further aspect, provided herein is a method of inducing the degradation of a protein in a cell, tissue, or biological sample, the method comprising administering to the cell, tissue, or biological sample an effective amount of a compound disclosed herein (e.g., a compound of Formula (I′) or (I)), or a pharmaceutically acceptable salt or tautomer thereof, or composition disclosed herein.

In a further aspect, provided herein is a kit comprising a compound disclosed herein (e.g., a compound of Formula (I′) or (I)), or a pharmaceutically acceptable salt or tautomer thereof, or composition disclosed herein and instructions for using the compound or composition.

The details of certain embodiments of the invention are set forth in the Detailed Description of Certain Embodiments, as described below. Other features, objects, and advantages of the invention will be apparent from the Definitions, Figures, Examples, and Claims.

### BRIEF DESCRIPTION OF THE DRAWINGS

FIGS. 1A-1I show that cyclic glutamine dipeptides functionally engage cereblon in targeted protein degradation. FIG. 1A shows an illustration of CRBN engaged by thalidomide and lenalidomide in a manner that may mimic the biological ligand. Models of CRBN engagement by either a small molecule or post-translational modification (PTM)-based degron. FIG. 1B shows the structure of thalidomide and candidate structures for the CRBN degron. FIG. 1C shows the structures of dBET6 and candidate dipeptide degraders JQ1-XcQ for functional engagement of CRBN and target protein degradation in cells. FIGS. 1D-1E show Western blots of BRD4 levels after treatment of HEK293T cells with 100 nM (FIG. 1D) or 10 μM (FIG. 1E) of dBET6 or the 20 dipeptide degraders. FIG. 1F shows a Western blot of BRD4 after treatment with dBET6 and selected dipeptide degraders over a 1-100 nM dose response range. FIG. 1G shows a Western blot of BRD4 after treatment of wild type (WT), shRNA knockdown (CRBN KD) HEK293T cells with the indicated degrader. FIG. 1H shows a Western blot of BRD4 after treatment of HEK293T cells with one of the three epimers of JQ1-FcQ at 100 nM. FIG. 1I shows a Western blot of BRD4 after co-treatment of HEK293T cells with JQ1-FcQ and lenalidomide or Boc-FcQ. Degradation assays were performed with 4 h incubation. Western blot data are representative of at least 2 independent replicates.

FIGS. 2A-2D show that cyclic asparagine dipeptides are functional substitutes of thalidomide in targeted protein degradation. FIG. 2A shows the structure of candidate dipeptide degraders JQ1-XcN. FIG. 2B shows a Western blot of BRD4 levels after treatment of HEK293T cells with 100 nM dBET6 or the indicated dipeptide degraders over 1-100 nM in the presence or absence of MLN4924. FIG. 2C shows a Western blot of BRD4 after treatment of wild type (WT) HEK293T cells or CRBN-KD cells. FIG. 2D shows a Western blot of BRD4 after co-treatment of HEK293T cells with JQ1-FcQ and lenalidomide or Boc-FcN. Degradation assays were performed with 4 h incubation. Western blot data are representative of at least two independent replicates.

FIGS. 3A-3C show the engagement of CRBN and ternary complex formation of dipeptide degraders. FIG. 3A shows a co-immunoprecipitation of BRD4 with FLAG-CRBN from HEK293T cells after 2 h incubation with 25 μM of the indicated degraders. Western blot data are representative of two independent replicates. FIG. 3B shows a AlphaScreen for ternary complex formation between GST-BRD4 and His-CRBN/DDB1 in the presence of varying concentrations of dBET6 or JQ1-FcQ. FIG. 3C shows a relative area under the curve from the AlphaScreen for ternary complex formation between GST-BRD4 and His-CRBN/DDB1 in the presence of the indicated members of the degrader library normalized to dBET6. AlphaScreen data is representative of three replicates.

FIGS. 4A-4J show that the dipeptide FcQ is a selective and transferrable substitute for the IMiDs in target protein degradation. FIG. 4A shows quantitative proteomics of MM.1S cells after treatment with 10 μM pomalidomide over 10 h. FIG. 4B shows quantitative proteomics of MM. 1S cells after treatment with 10 μM of Boc-FcQ over 10 h. Global proteomics experiments were performed in biological triplicate. FIG. 4C shows a Western blot of IKZF1 levels after treatment with 10 μM of the indicated compound in MM.1S cells. FIG. 4D shows results of a cell viability (MTT) assay of the indicated compounds after treatment of MM.1S cells for 5 d. FIG. 4E shows the structure of FKBP12 degraders dFKBP-1 and dFKBP-FcQ. FIG. 4F shows a Western blot of FKBP12 levels after treatment of HEK293T cells with dFKBP-1 or dFKBP-FcQ over a 10 nM-10 UM dose response range. FIG. 4G shows a Western blot of FKBP levels after co-treatment of HEK293T cells with JQ1-FcQ and lenalidomide or Boc-FcQ. Western blot data are representative of three independent replicates. FIG. 4H shows the structure of CDK6 degraders dCDK6-Pom and dCDK6-FcQ. FIG. 4I shows a Western blot of CDK4/6 levels after treatment of Jurkat cells with dCDK6-Pom or dCDK6-FcQ over a 0.01-10 UM dose response range. FIG. 4J shows a Western blot of CDK6 levels after co-treatment of Jurkat cells with dCDK6-FcQ and lenalidomide or Boc-FcQ. FIG. 4K shows the structures of dCDK-PEG2-FcQ, dCDK-C4-FcQ, and dCDK-C6-FcQ. FIG. 4L shows a Western blot of CDK4/6 levels after treatment of Jurkat with various concentrations of dCDK-PEG2-FcQ, dCDK-C4-FcQ, or dCDK-C6-FcQ. FIG. 4M shows a Western blot of FKBP levels after treatment of HEK293T cells with 1 μM or 0.5 μM of dFKBP-XcN or dFKBP-XcQ.

FIGS. 5A-5J show that proteins with C-terminal cGln or cAsn are substrates of CRBN for ubiquitylation and degradation. FIG. 5A shows in vitro ubiquitylation of GFP tagged with C-terminal cyclic imide. GFP-G=GFP with C-terminal GGG; GFP-Me=GFP with C-terminal FcQMe; GFP-FcQ=GFP with C-terminal FcQ; GFP-FcN=GFP with C-terminal FcN. FIG. 5B shows in vitro ubiquitylation of GFP tagged with C-terminal glutamine. FIG. 5C shows a flow cytometry analysis of the % GFP-positive HEK293T cells 6 h after electroporation with GFP tagged with the indicated peptide. GFP-His₆=GFP with C-terminal His₆ tag (no sortase treatment). Each condition was assayed in triplicate. FIG. 5D shows a flow cytometry analysis of the % GFP-positive HEK293T cells 6 h after electroporation with GFP-FcQ with or without lenalidomide competition (100 μM). Each condition was assayed in triplicate. FIG. 5E shows in vitro ubiquitylation of FKBP12 tagged with C-terminal cyclic imide. FKBP12-H6=GFP with C-terminal His₆ tag (no sortase treatment); FKBP12-Me=FKBP12 with C-terminal FcQMe; FKBP12-FcQ=FKBP12 with C-terminal FcQ; FKBP12-FcN=FKBP12 with C-terminal FcN. FIG. 5F shows a Western blot of FKBP12 tagged with the indicated peptide after electroporation into HEK293T cells. FIG. 5G shows a schematic of cyclic imide formation via intramolecular cyclization and cleavage of the protein backbone. Formation of the cyclic imide reveals a degron for CRBN and promotes protein degradation. FIGS. 5H-5I show proteins (FIG. 5H) and peptides (FIG. 5I) that have at least one cN or cQ modification from global proteomics datasets. FIG. 5J shows a representative spectrum of hemoglobin peptide bearing C-terminal aspartimide from red blood cells (SEQ ID NO: 2). ns=not significant, *=p<0.05, **=p<0.01, ***=p<0.001, ****=p<0.0001; Western blot data are representative of at least two independent replicates.

FIGS. 6A-6D show an examination of bifunctional degraders of BRD4 with candidate degrons for CRBN. FIG. 6A shows the structures of JQ1-uracil, JQ1-PEG-uracil, and JQ1-uridine. FIG. 6B shows a Western blot of BRD4 after treatment with JQ1-uracil, JQ1-PEG-uracil, or JQ1-uridine in HEK293T cells over 24 h. FIG. 6C shows the structures of JQ1-cQ, JQ1-cN, and JQ1-FpE. FIG. 6D shows a Western blot of BRD4 after treatment with JQ1-cQ, JQ1-cN, or JQ1-pE in HEK293T cells over 4 h. Western blot data are representative of at least two independent replicates.

FIGS. 7A-7C show the examination of dipeptides as functional degraders of BRD4. FIG. 7A shows an evaluation of JQ1-XcQ degraders in targeted protein degradation of BRD4 at 1 μM concentrations in HEK293T cells over 4 h. FIG. 7B shows a dBET6 degradation of BRD4 over 4 h is competitively inhibited by lenalidomide or Boc-FcQ in a dose dependent manner over concentrations of 0.1-100 μM. FIG. 7C shows levels of BRD4 over time in HEK293T cells treated with dBET6 or JQ1-FcQ. Western blot data are representative of at least two independent replicates.

FIGS. 8A-8B show the evaluation of JQ1-XcN degraders in targeted protein degradation of BRD4. FIG. 8A shows the evaluation of JQ1-HcN in targeted protein degradation of BRD4 at various concentrations in HEK293T cells over 4 h. FIG. 8B shows levels of BRD4 over time in HEK293T cells treated with JQ1-FcN. Western blot data are representative of two independent replicates.

FIG. 9 shows co-immunoprecipitation of BRD4 with FLAG-CRBN from HEK293T lysates in the presence of 1 μM of the indicated degraders. Lysates of HEK293T-CRBN were incubated with the indicated degrader and 1 μM MLN4924 for 2 h prior to immunoprecipitation with anti-FLAG magnetic beads.

FIGS. 10A-10W show AlphaScreen experiments with the CULT domain of CRBN and BRD4. FIGS. 10A-10B show a schematic showing the domains of cereblon, including the CULT domain with residues for immunomodulatory drug binding highlighted (FIG. 10A) (SEQ ID NOs: 2-4), and AlphaScreen design (FIG. 10B). FIGS. 10C-10L and FIGS. 10M-10U show AlphaScreen experiments performed with the indicated degrader compounds. Data shown in FIGS. 10C-10L and FIGS. 10M-10U are each performed on a single 384-well plate; dBET6 was assayed on each plate as an internal control. Each condition was measured in triplicate. FIG. 10V shows a schematic of a NanoBRET assay. FIG. 10W shows the NanoBRET measurement of ternary complex formation induced by the indicated members of the degrader library as determined by acceptor:donor signal ratio, with background signal from no-ligand control for each degrader subtracted. Each condition was assayed in triplicate. Error bars represent SEM.

FIGS. 11A-11W show models of dipeptide degraders with CRBN built from 6BOY and 6H0F. FIG. 11A shows models of JQ1-FcQ (yellow) by molecular replacement of dBET6 (orange) in the ternary complex with CRBN (grey) and BRD4 (blue). FIG. 11B shows a zoom in of the CRBN binding pocket with thalidomide analog (orange), Ac-FcQ (yellow), or Ac-FcN (green). FIG. 11C shows a model of lenalidomide (orange), FcQ (yellow), and FcN (green) in the ternary complex with CRBN (grey) and IKZF1 (blue, space filling mode). FIGS. 11D-11W show models of the 20 glutarimide dipeptides in the CRBN binding pocket.

FIGS. 12A-12F show the characterization of the IMiDs and the indicated dipeptides in HEK293T cells and multiple myeloma MM.1S cells. FIG. 12A shows competitive inhibition of BRD4 degradation by the indicated dipeptide degrader (JQ1-FcN, JQ1-FcQ) with the indicated compounds in HEK293T cells for 4 h. FIG. 12B shows a quantitative proteomics of MM.1S cells after treatment with 10 μM of Boc-FcN for 10 h. FIG. 12C shows protein expression levels of IKZF1 after treatment with the indicated compounds in MM.1S cells for 10 h. FIG. 12D shows a quantitative proteomics of HEK293T cells after treatment with 0.1 μM of JQ1-FcQ or dBET6 for 2 h. FIG. 12E shows protein expression levels of BRD2 and BRD4 after treatment with the indicated compounds in HEK293T cells for 2 h. FIG. 12F shows a time course of FKBP12 degradation by the indicated degraders in HEK293T cells.

FIGS. 13A-13F show the design and characterization of degron-tagged semi-synthetic proteins as CRBN substrates. FIG. 13A shows a sortase system used to generate degron-tagged GFP from GFP-LPETG-His₆ (SEQ ID NOs: 5-6). FIG. 13B shows intact MS measurements of the indicated semi-synthetic GFP proteins FIG. 13C shows a sortase system used to generate degron-tagged GST-FKBP12 from GST-FKBP12-LPETG-His₆ (SEQ ID NO: 5, 7). FIG. 13D shows intact MS measurements of the indicated semi-synthetic GST-FKBP12 proteins FIG. 13E shows hydrolysis of cyclic imides Fmoc-GGGFcQ (SEQ ID NO: 8) or Fmoc-GGGFcN (SEQ ID NO: 9) in PBS at 37° C. FIG. 13F shows hydrolysis of C-terminal cyclic imides on GFP-FcQ or GFP-FcN in PBS at 37° C.

FIGS. 14A-14E show the analysis of cQ/cN modifications on hemoglobin derived from red blood cell lysates. FIG. 14A shows a Western blot of red blood cell lysates from two donors in comparison to HEK293T lysate. Red blood cells do not express CRBN. FIG. 14B shows representative spectra of peptides containing cN detected in hemoglobin subunits alpha and beta (SEQ ID NOs: 1, 10, 11). FIG. 14C shows a comparison of peptide spectral matches (PSMs) for hemoglobin subunits observed in global proteomics datasets and red blood cell (RBC) lysates. Lower-case n and q represent the dehydrated cyclic imide modification (SEQ ID NO: 12-20). FIG. 14D shows a schematic of the sequences of hemoglobin subunits. The peptides with cN modifications observed by MS analysis are underlined (global datasets=blue, red blood cell donors=pink); and the modification sites are highlighted in red (SEQ ID NO: 21-22). FIG. 14E shows ion intensity chromatograms extracted for the tryptic peptide and post-translationally modified peptides from red blood cell lysates. These peptides do not have the same retention time (SEQ ID NOs: 1, 11, 23, 24).

FIGS. 15A-15I show that C-terminal cGln and cAsn are degrons that promote CRBN-dependent ubiquitylation and degradation. FIG. 15A shows a quantification of ubiquitylated protein band intensity in experiment shown in FIG. 5A across three replicates. FIG. 15B shows in vitro ubiquitylation of GFP tagged with uncyclized C-terminal glutamine and asparagine. FIG. 15C shows a quantification of ubiquitylated protein band intensity in experiment shown in FIG. 15B across three replicates. FIG. 15D shows a flow cytometry analysis of the GFP levels in WT or CRBN KO HEK293T cells 6 hours after electroporation with GFP tagged with the indicated peptide. FIG. 15E shows a flow cytometry analysis of the GFP levels in HEK293T cells 6 hours after electroporation with GFP tagged with the indicated peptide, with or without lenalidomide competition (100 μM). GFP-His₆=GFP with C-terminal His₆ tag (no sortase treatment). FIG. 15F shows a flow cytometry analysis of the GFP levels in HEK293T cells 6 hours after electroporation with GFP tagged with the indicated peptide, with C-terminal Q and N cyclized and uncyclized. FIG. 15G shows a flow cytometry analysis of the GFP levels in HEK293T cells 6 hours after electroporation with GFP tagged with the indicated peptide with or without lenalidomide competition (100 μM).

FIG. 15H shows a flow cytometry analysis of the GFP levels in Jurkat cells 6 hours after electroporation with GFP tagged with the indicated peptide, with or without lenalidomide competition (100 μM). FIG. 15I shows a flow cytometry analysis of the GFP levels in MEF cells 6 hours after electroporation with GFP tagged with the indicated peptide, with or without lenalidomide competition (100 μM). Comparisons were performed using a one-way ANOVA with Šídák's multiple comparisons test. ns=not significant, *=p<0.05, **=p<0.01, ***=p<0.001, ****=p<0.0001. All Western blot data and flow cytometry data are representative of three independent replicates.

FIGS. 16A-16F show that CRBN regulates endogenous substrates bearing C-terminal cyclic imides. FIG. 16A shows a comparison of peptide spectral matches (PSMs) for hemoglobin subunits observed in global proteomics datasets and RBC lysates (SEQ ID NOs: 25-32). HBA[63-69cN] was observed by extracted ion chromatogram in the MS1. FIG. 16B shows quantification of the three major peptide groups bearing C-terminal cyclic imides in RBC samples with or without base treatment. Proteomics experiments were performed in biological triplicate. FIG. 16C shows in vitro time course for formation of the cyclic imide fragment (cN) and the hydrolysis products on the synthetic peptide corresponding to hemoglobin beta residues 42-60. The peptide was incubated in 20 mM ammonium acetate buffer at 37° C. over pH 7.4-9.0. FIG. 16D shows percentage of the formed cyclic imide fragment (CN) and the hydrolysis products at the indicated residue relative to the parent synthetic peptide at different pH. FIG. 16E shows volcano plots of peptide groups bearing C-terminal cyclic imides in WT HEK293T, CRBN CRISPR/Cas9 knockout HEK293T, or HEK293T treated with 200 μM lenalidomide over 48 hours. Upregulated proteins at 1% FDR=red, 5% FDR=pink. FIG. 16F shows volcano plots of peptide groups bearing C-terminal cyclic imides in MM.1S treated with DMSO or 200 μM lenalidomide over 48 hours. Upregulated proteins at 1% FDR=red, 5% FDR=pink.

FIGS. 17A-17C show the design and characterization of degron-tagged semi-synthetic GFP as CRBN substrates. FIG. 17A shows Western blots of in vitro ubiquitylation of GFP tagged with the indicated peptides quantified in FIG. 4F. FIG. 17B shows Western blots of in vitro ubiquitylation of GFP tagged with the indicated peptides quantified in FIG. 4H. FIG. 17C shows intact MS measurements of the indicated semi-synthetic GFP proteins.

FIGS. 18A-18D demonstrate the analysis of cQ/cN modifications on hemoglobin derived from red blood cells and beta-crystallin derived from bovine lens. FIG. 18A (left) shows hydrolysis of cyclic imides Fmoc-GGGFcQ (SEQ ID NO: 8) or Fmoc-GGGFcN (SEQ ID NO: 9) in PBS at 37° C. Right: hydrolysis of C-terminal cyclic imides on GFP-FcQ or GFP-FcN in PBS at 37° C. FIG. 18B demonstrates representative spectra of peptides containing cN detected in hemoglobin subunits alpha and beta (SEQ ID NOs: 1, 10, 11).

FIG. 18C shows ion intensity chromatogram extracted for the masses of the parent tryptic peptide and the cyclic imide fragment for red blood cell lysates and the synthetic peptide labeled with TMT-10plex reagent (sequence shown below the chromatogram). The synthetic peptide shows completely overlapping retention time with the internal cleavage product indicative of the in situ formation while the RBC sample shows a different retention time for the cyclic imide fragment (SEQ ID NOs: 34-35). FIG. 18D shows ion intensity chromatograms extracted for the masses of the cyclic imide fragment and the corresponding tryptic peptide for three cyclic imide-bearing peptide groups identified in bovine lens. Quantification of these peptide groups validates the sensitivity of the cyclic imide modifications to base treatment. Proteomics experiments were performed in biological quadruplicate.

FIGS. 19A-19F shows the analysis of cQ/cN and Q/N modifications on synthetic peptides and in cell lines. FIG. 19A demonstrates the scheme of cyclic imide formation in a peptide and subsequent hydrolysis to afford the truncated C-terminal glutamine or asparagine fragments. FIG. 19B shows a representative overlay of extracted ion chromatograms of each peptide at the masses corresponding to parent peptide (black), cyclic imide fragment (red), and its hydrolyzed forms (blue). The two constitutional isomers formed via hydrolysis of the cyclic imide fragment were not distinguished in our study. The three species were largely observed at distinct retention times (SEQ ID NOs: 10, 34-43). FIG. 19C demonstrates in vitro time course for formation of the cyclic imide fragment (CN) and the hydrolysis products at the indicated position on the synthetic peptide after incubation. The peptides were incubated in 20 mM ammonium acetate buffer at 37° C., and at pH 7.4-9.0. FIG. 19D shows raw extracted ion chromatograms for the m z of the cyclic imide fragment HBB[42-58] (908.39-908.41) from the peptide formation study (upper) and from a label-free RBC digest (lower) run on the same liquid chromatography gradient. The extracted peaks were observed at the same retention time. FIG. 19E shows volcano plots of peptide groups bearing C-terminal glutamine or asparagine (protein terminus excluded) in WT HEK293T, CRBN CRISPR/Cas9 knockout HEK293T, or HEK293T treated with 200 μM lenalidomide over 48 h. Upregulated peptide groups at 1% FDR=red, 5% FDR=pink. FIG. 19F shows volcano plots of peptide groups bearing C-terminal glutamine or asparagine (protein terminus excluded) in MM.1S treated with DMSO or 200 μM lenalidomide over 48 hours. Upregulated peptide groups at 1% FDR=red, 5% FDR=pink.

FIGS. 20A-C show Western blots of BRD4 levels after treatment of HEK293T cells with 100 nM (FIG. 20A) and 10 μM (FIG. 20B) of dBET6 or the 20 JQ1 dipeptide degraders (JQ1-XcN). FIG. 20C shows a Western blot of HEK293T cells with different dipeptide degraders (JQ1-XcN) at version concentrations.

FIG. 21 shows a schematic of in vitro TR-FRET assay.

FIG. 22A shows the results of cellular degradation assay of BRD4 BD1 and BD2 demonstrating that cyclimid degraders preferentially from the ternary complex with BD1 compared to BD2. FIG. 22B shows the metabolic stability of cyclimids. FIG. 22C shows the results of in vitro permeability assay in Caco-2 cells.

FIGS. 23A-23E show degron-inspired CRBN ligands, cyclimids, exhibit distinct and diverse binding affinities against CRBN compared to IMiDs. FIG. 23A shows CRBN recognizes C-terminal cyclic imide degrons, which are mimicked by thalidomide and lenalidomide. Structures of immunomodulatory drugs (IMiDs) and C-terminal cyclic imide degron (XcQ and XcN, collectively called cyclimids). Cyclimids, a class of CRBN ligands inspired by degron, may serve as alternative ligands in the development of PROTACs. FIG. 23B are structures of dBET6 and cyclimid degraders JQ1-XcQ and JQ1-XcN for functional engagement of CRBN and BRD4 degradation in cells. FIG. 23C is a schematic of the TR-FRET assay. FIG. 23D show KD values of the indicated compounds against CRBN/DDB1 complex measured by TR-FRET assay. FIG. 23E show the comparison between cQ- and cN-cyclimids or IMiDs in terms of pKD values against CRBN/DDB1. In contrast to IMiDs, cN-cyclimids bind to CRBN/DDB1 more tightly than their cQ counterparts.

FIGS. 24A-24D show cyclimid degraders induce interprotein contacts that are sensitive and distinct from IMiD-based degraders. FIG. 24A is a schematic of the TR-FRET assay principle for determining KD (binary) and KD (ternary) against CRBN/DDB1. FIG. 24B shows a comparison of pKD (ternary) values against CRBN/DDB1 in the presence of BD1 or BD2 domain of BRD4. KD (ternary) values of the indicated compounds were measured using the TR-FRET assay in the presence of either BD1 or BD2 domain of BRD4.

FIG. 24C is a schematic of the cooperativity factor Alpha. FIG. 24D shows a comparison of log(Alpha) values in the presence of BD1 or BD2 domain of BRD4. Log(Alpha) values for the cyclimids with the BD1 domain of BRD4 showed great variance depending on the amino acid at the N−1 position.

FIGS. 25A-25F show pKD (ternary) values measured with TR-FRET correlate with degradation ability of cyclimid degraders. FIG. 25A shows the selectivity parameter between BD1 and BD2 degradation of IMiD-based or cyclimid BRD4 degraders. Cyclimid degraders display higher selectivity than dBET6. FIG. 25B show the alpha values of JQ1-YcQ were measured in the presence of either the BD1 or BD2 domains of BRD4. FIGS. 25C-25E show BRD4 protein levels in MDA-MB-231 cell lysate after 5 hour treatment with dBET6 and the selected cyclimid BRD4 degraders were measured by TR-FRET assay. FIG. 25F is a correlation between pDC50 and the pKD (ternary) of BRD4.

FIGS. 26A-26C show the interrogation of ligand-, time-, and concentration-dependent dynamics of CRBN/DDB1 or BRD4 dissociation from the ternary complex. FIG. 26A is a schematic illustration of TR-FRET assay system employed to monitor CRBN/DDB1 or BRD4 dissociation and association. FIG. 26B shows kinetic measurements of CRBN/DDB1 dissociation in the presence of BD1 or BD2 domains of BRD4. t_{1/2} values of the indicated compounds were measured by TR-FRET assay. FIG. 26C show kinetic measurements of BRD4 dissociation in the presence of the BD1 or BD2 domain of BRD4. t_{1/2} values of the indicated compounds were measured by TR-FRET assay.

FIGS. 27A-27H show the cyclimid library can be readily applied to targeting other proteins for degradation. FIG. 27A depict a structure of dFKBP-1 and cyclimid-based FKBP degraders, dFKBP-cyclimid. FIG. 27B are the pKD (ternary) values of the indicated compounds against CRBN/DDB1 in the presence of FKBP12. FIG. 27C shows a western blot of FKBP12, FKBP51, and FKBP52 levels after treatment of HEK293T cells with 10 μM dFKBP-1 or the dFKBP-cyclimid. FIG. 27D shows a structure of dCDK-1 and cyclimid-based CDK degraders, dCDK-cyclimid. FIG. 27E shows a western blot of CDK4 and CDK6 levels after treatment of Jurkat cells with 10 μM dCDK-1 or the dCDK-cyclimid. FIG. 27F shows a western blot of CDK4 and CDK6 levels after treatment of Jurkat cells with 1 μM dCDK-1 or the dCDK-cyclimid. FIG. 27G shows a western blot of CDK4 and CDK6 levels after treatment of Jurkat cells with 0.1 μM dCDK-1 or the dCDK-cyclimid. FIG. 27H shows a western blot of CDK4 and CDK6 levels after treatment of Jurkat cells with 0.01 μM dCDK-1 or the dCDK-cyclimid.

FIGS. 28A-28F show cyclimids do not have inherent off-target effects via the recruitment of neosubstrates unlike IMiDs. FIG. 28A shows degradation of validated and pomalidomide-sensitive ZF degrons in cells by reported IMID-based and cyclimid BRD4 degraders in a dosage range of 32 nM to 20 μM. U2OS cells stably expressing 6 ZF degrons fused to eGFP were treated with PROTACs followed by flow cytometry to assess ZF degradation. FIG. 28B shows degradation of GSPT1 in HEK293T cells using IMID-based and cyclimid BRD4 degraders. FIG. 28C is a schematic of the suppression of off-target degradation by cyclimids. FIG. 28D shows a western blot of indicated proteins after treatment of MM.1S cells with one of the IMiD-based or cyclimid BRD4 degraders. IMID-based degraders induce off-target degradation that is substantially decreased by cyclimid degraders. FIG. 28E shows degradation of pomalidomide-sensitive ZF degrons in cells by IMID- or cyclimid-based FKBP and CDK degraders in a dosage range of 32 nM to 20 μM. U2OS cells stably expressing 2 ZF degrons fused to eGFP were treated with PROTACs followed by flow cytometry to assess ZF degradation. FIG. 27F shows a western blot of indicated proteins after treatment of MM.1S cells with one of IMID- or cyclimid-based FKBP and CDK degraders. Cyclimid degraders suppress off-target degradation induced by IMID-based degraders.

### DEFINITIONS

For convenience, certain terms employed herein, in the specification, examples, and claims are collected herein.

Unless otherwise required by context, singular terms shall include pluralities, and plural terms shall include the singular.

The language “in some embodiments” and the language “in certain embodiments” are used interchangeably.

The following definitions are more general terms used throughout the present application:

The singular terms “a,” “an,” and “the” include plural references unless the context clearly indicates otherwise. Similarly, the word “or” is intended to include “and” unless the context clearly indicates otherwise.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7th Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3rd Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high-performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFC₁₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

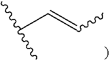

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5, 7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR)₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(ORC^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR)₂, —OP(OR)₃⁺X⁻, —OP(R^{cc})₄, —OP(OR^{cc})₄, —B(R^{aa})₂, —B(OR)₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR;
wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, CN, —NO₂, —N₃, —SO₂H, —SO₃H, —OH, OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)Rec, —NR^{ff}CO₂R^{ee}, —NR^{ff}C(═O)N(R^{ff})₂, —C(═NR^{ff})OR^{ee}, OC(═NR^{ff})Rec, —OC(═NR^{ff})OR^{ee}, —C(═NR^{ff})N(R^{ff})₂, —OC(═NR^{ff})N(R^{ff})₂, —NR^{ff}C(—NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), OC(═O)(C₁₋₆ alkyl), —OCO(C₁₋₆ alkyl), C(═O)NH₂, C(═O)N(C₁₋₆ alkyl)₂, OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), OC(═NH)OC₁₋₆ alkyl, —C(═NH)N(C₁₋₆ alkyl)₂, C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R& substituents can be joined to form ═O or ═S; and
each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —C₁), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR)₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR)₂, and —NR^{bb}P(═O)(N(R^{bb})₂)₂, wherein R^{aa}, R^{bb}, and Rec are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{cc}, —C(═NR^{cc})N(R^{cc})₂, —SO₂N(R^{cc})₂, —SO₂R^{cc}, —SO₂OR, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(0-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o)-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), 1-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), 1-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, 1-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o)-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb}) 2)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “amorphous” or “amorphous form” refers to a form of a solid (“solid form”), the form substantially lacking three-dimensional order. In certain embodiments, an amorphous form of a solid is a solid form that is substantially not crystalline. In certain embodiments, the X-ray powder diffraction (XRPD) pattern of an amorphous form includes a wide scattering band with a peak at 2θ of, e.g., between 20 and 70°, inclusive, using CuKα radiation. In certain embodiments, the XRPD pattern of an amorphous form further includes one or more peaks attributed to crystalline structures. In certain embodiments, the maximum intensity of any one of the one or more peaks attributed to crystalline structures observed at a 2θ of between 20 and 70°, inclusive, is not more than 300-fold, not more than 100-fold, not more than 30-fold, not more than 10-fold, or not more than 3-fold of the maximum intensity of the wide scattering band. In certain embodiments, the XRPD pattern of an amorphous form includes no peaks attributed to crystalline structures.

The term “co-crystal” refers to a crystalline structure comprising at least two different components (e.g., a compound disclosed herein (e.g., a compound of Formula (I′) or (I)) and an acid), wherein each of the components is independently an atom, ion, or molecule. In certain embodiments, none of the components is a solvent. In certain embodiments, at least one of the components is a solvent. A co-crystal of a compound disclosed herein (e.g., a compound of Formula (I′) or (I)) and an acid is different from a salt formed from a compound disclosed herein (e.g., a compound of Formula (I′) or (I)) and the acid. In the salt, a compound disclosed herein (e.g., a compound of Formula (I′) or (I)) is complexed with the acid in a way that proton transfer (e.g., a complete proton transfer) from the acid to a compound disclosed herein (e.g., a compound of Formula (I′) or (I)) easily occurs at room temperature. In the co-crystal, however, a compound disclosed herein (e.g., a compound of Formula (I′) or (I)) is complexed with the acid in a way that proton transfer from the acid to a compound disclosed herein (e.g., a compound of Formula (I′) or (I)) does not easily occur at room temperature. In certain embodiments, in the co-crystal, there is no proton transfer from the acid to a compound disclosed herein (e.g., a compound of Formula (I′) or (I)). In certain embodiments, in the co-crystal, there is partial proton transfer from the acid to a compound disclosed herein (e.g., a compound of Formula (I′) or (I)). Co-crystals may be useful to improve the properties (e.g., solubility, stability, and ease of formulation) of a compound disclosed herein (e.g., a compound of Formula (I′) or (I)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The term “amino acid” refers to naturally occurring and synthetic amino acids, as well as amino acid analogs and amino acid mimetics that function in a manner similar to the naturally occurring amino acids. Every amino acid contains an amine (—NH₂) and a carboxylic acid (—COOH) functional group. Each amino acid contains a unique side chain, designated by the “R” substituent shown below.

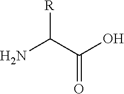

Naturally occurring amino acids are those encoded by the genetic code, as well as those amino acids that are later modified, e.g., hydroxyproline, γ-carboxyglutamate, and O-phosphoserine. In certain embodiments, the amino acid is an N-alkyl amino acid, where the hydrogen on any non-proline amine (N) is replaced with an alkyl (e.g., methyl (—CH₃)) group. In certain embodiments, the N-alkyl amino acid is sarcosine (Sar). Amino acid analogs refer to compounds that have the same basic chemical structure as a naturally occurring amino acid, i.e., a carbon that is bound to a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Unnatural (or non-natural) amino acids refer to those not naturally incorporated into proteins during translation. Examples of unnatural amino acids include, but are not limited to, β-amino acids (e.g., β² and β³), homo-amino acids, proline derivatives, pyruvic acid derivatives, alanine derivatives (e.g., 1′- and 2′-naphthylalanine), glycine derivatives, ring-substituted phenylalanine and tyrosine derivatives, linear core amino acids, and N-methyl amino acids. Amino acids may be referred to herein by either their commonly known three letter symbols or by the one-letter symbols recommended by the IUPAC-IUB Biochemical Nomenclature Commission. As to amino acid sequences, one of skill will recognize that individual substitutions to a peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. In certain embodiments, a compound of Formula (I′) or (I) provided herein comprises an amino acid side chain selected from the 20 proteinogenic amino acids (i.e., an amino acid incorporated into proteins during translation) shown in Table A. The term amino acid may also refer to non-proteinogenic amino acids, such as, for example, selenocysteine (—CH₂SeH).

TABLE A
Amino acids and side chains.

| | 3- | | Side chain |
|---|---|---|---|
| | letter | | classification |
| Amino Acid | code | Side chain (R group) | (at pH 7.4) |
| Arginine | Arg | —(CH₂)₃NHC(NH)NH₂ | Charged (Positive) |
| Histidine | His | —CH₂—C₃H₃N₂ | |
| Lysine | Lys | —(CH₂)₄NH₂ | |
| Aspartic acid | Asp | —CH₂COOH | Charged (Negative) |
| (aspartate) | | | |
| Glutamic acid | Glu | —CH₂CH₂COOH | |
| (glutamate) | | | |
| Serine | Ser | —CH₂OH | Uncharged |
| Threonine | Thr | —CH(OH)CH₃ | |
| Asparagine | Asn | —CH₂CONH₂ | |
| Glutamine | Gln | —CH₂CH₂CONH₂ | |
| Cysteine | Cys | —CH₂SH | Special |
| Glycine | Gly | —H | |
| Proline | Pro | —CH₂CH₂CH₂— | Hydrophobic |
| Alanine | Ala | —CH₃ | |
| Isoleucine | Ile | —CH(CH₃)CH₂CH₃ | |
| Leucine | Leu | —CH₂CH(CH₃)₂ | |
| Methionine | Met | —CH₂CH₂SCH₃ | |
| Phenylalanine | Phe | —CH₂C₆H₅ | |
| Tryptophan | Trp | —CH₂C₈H₆N | |
| Tyrosine | Tyr | —CH₂—C₆H₄OH | |
| Valine | Val | —CH(CH₃)₂ | |

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “E3 ubiquitin ligase” or “E3 ligase” refers to any protein that recruits an E2 ubiquitin-conjugating enzyme that has been loaded with ubiquitin, recognizes a protein substrate, and assists or directly catalyzes the transfer of ubiquitin from the E2 protein to the protein substrate.

A “kinase” is a type of enzyme that transfers phosphate groups from high energy donor molecules, such as ATP, to specific substrates, referred to as phosphorylation. Kinases are part of the larger family of phosphotransferases. One of the largest groups of kinases are protein kinases, which act on and modify the activity of specific proteins. Kinases are used extensively to transmit signals and control complex processes in cells. Various other kinases act on small molecules such as lipids, carbohydrates, amino acids, and nucleotides, either for signaling or to prime them for metabolic pathways. Kinases are often named after their substrates. More than 500 different protein kinases have been identified in humans. These exemplary protein kinases include, but are not limited to, AAK1, ABL, ACK, ACTR2, ACTR2B, AKT1, AKT2, AKT3, ALK, ALK1, ALK2, ALK4, ALK7, AMPKa1, AMPKa2, ANKRD3, ANPa, ANPb, ARAF, ARAFps, ARG, AurA, AurAps1, AurAps2, AurB, AurBps1, AurC, AXL, BARK1, BARK2, BIKE, BLK, BMPRIA, BMPRIAps1, BMPRIAps2, BMPRIB, BMPR2, BMX, BRAF, BRAFps, BRK, BRSK1, BRSK2, BTK, BUB1, BUBR1, CaMK1a, CaMK1b, CaMK1d, CaMK1g, CaMK2a, CaMK2b, CaMK2d, CaMK2g, CaMK4, CaMKK1, CaMKK2, caMLCK, CASK, CCK4, CCRK, CDC2, CDC7, CDK10, CDK11, CDK1, CDK2, CDK3, CDK4, CDK4ps, CDK5, CDK5ps, CDK6, CDK7, CDK7ps, CDK8, CDK8ps, CDK9, CDKL1, CDKL2, CDKL3, CDKL4, CDKL5, CGDps, CHED, CHK1, CHK2, CHK2ps1, CHK2ps2, CK1a, CK1a2, CK1aps1, CK1aps2, CK1aps3, CK1d, CK1e, CK1g1, CK1g2, CK1g2ps, CK1g3, CK2al, CK2a1-rs, CK2a2, CLIK1, CLIKIL, CLK1, CLK2, CLK2ps, CLK3, CLK3ps, CLK4, COT, CRIK, CRK7, CSK, CTK, CYGD, CYGF, DAPK1, DAPK2, DAPK3, DCAMKL1, DCAMKL2, DCAMKL3, DDR1, DDR2, DLK, DMPK1, DMPK2, DRAK1, DRAK2, DYRKIA, DYRKIB, DYRK2, DYRK3, DYRK4, EGFR, EphA1, EphA10, EphA2, EphA3, EphA4, EphA5, EphA6, EphA7, EphA8, EphB1, EphB2, EphB3, EphB4, EphB6, Erk1, Erk2, Erk3, Erk3ps1, Erk3ps2, Erk3ps3, Erk3ps4, Erk4, Erk5, Erk7, FAK, FER, FERps, FES, FGFR1, FGFR2, FGFR3, FGFR4, FGR, FLT1, FLT1ps, FLT3, FLT4, FMS, FRK, Fused, FYN, GAK, GCK, GCN2, GCN22, GPRK4, GPRK5, GPRK6, GPRK6ps, GPRK7, GSK3A, GSK3B, Haspin, HCK, HER2/ErbB2, HER3/ErbB3, HER4/ErbB4, HH498, HIPK1, HIPK2, HIPK3, HIPK4, HPK1, HR1, HRIps, HSER, HUNK, ICK, IGF1R, IKKa, IKKb, IKKe, ILK, INSR, IRAK1, IRAK2, IRAK3, IRAK4, IRE1, IRE2, IRR, ITK, JAK1, JAK2, JAK3, JNK1, JNK2, JNK3, KDR, KHS1, KHS2, KIS, KIT, KSGCps, KSR1, KSR2, LATS1, LATS2, LCK, LIMK1, LIMK2, LIMK2ps, LKB1, LMR1, LMR2, LMR3, LOK, LRRK1, LRRK2, LTK, LYN, LZK, MAK, MAP2K1, MAP2KIps, MAP2K2, MAP2K2ps, MAP2K3, MAP2K4, MAP2K5, MAP2K6, MAP2K7, MAP3K1, MAP3K2, MAP3K3, MAP3K4, MAP3K5, MAP3K6, MAP3K7, MAP3K8, MAPKAPK2, MAPKAPK3, MAPKAPK5, MAPKAPKps1, MARK1, MARK2, MARK3, MARK4, MARKps01, MARKps02, MARKps03, MARKps04, MARKps05, MARKps07, MARKps08, MARKps09, MARKps10, MARKps11, MARKps12, MARKps13, MARKps15, MARKps16, MARKps17, MARKps18, MARKps19, MARKps20, MARKps21, MARKps22, MARKps23, MARKps24, MARKps25, MARKps26, MARKps27, MARKps28, MARKps29, MARKps30, MAST1, MAST2, MAST3, MAST4, MASTL, MELK, MER, MET, MISR2, MLK1, MLK2, MLK3, MLK4, MLKL, MNK1, MNKIps, MNK2, MOK, MOS, MPSK1, MPSK1ps, MRCKa, MRCKb, MRCKps, MSK1, MSK12, MSK2, MSK22, MSSK1, MST1, MST2, MST3, MST3ps, MST4, MUSK, MYO3A, MYO3B, MYT1, NDR1, NDR2, NEK1, NEK10, NEK11, NEK2, NEK2ps1, NEK2ps2, NEK2ps3, NEK3, NEK4, NEK4ps, NEK5, NEK6, NEK7, NEK8, NEK9, NIK, NIM1, NLK, NRBP1, NRBP2, NuaK1, NuaK2, Obscn, Obscn2, OSR1, p38a, p38b, p38d, p38g, p70S6K, p70S6Kb, p70S6Kps1, p70S6Kps2, PAK1, PAK2, PAK2ps, PAK3, PAK4, PAK5, PAK6, PASK, PBK, PCTAIRE1, PCTAIRE2, PCTAIRE3, PDGFRa, PDGFRb, PDK1, PEK, PFTAIRE1, PFTAIRE2, PHKg1, PHKg1ps1, PHKg1ps2, PHKg1ps3, PHKg2, PIK3R4, PIM1, PIM2, PIM3, PINK1, PITSLRE, PKACa, PKACb, PKACg, PKCa, PKCb, PKCd, PKCe, PKCg, PKCh, PKCi, PKCips, PKCt, PKCz, PKD1, PKD2, PKD3, PKG1, PKG2, PKN1, PKN2, PKN3, PKR, PLK1, PLK1ps1, PLK1ps2, PLK2, PLK3, PLK4, PRKX, PRKXps, PRKY, PRP4, PRP4ps, PRPK, PSKH1, PSKHIps, PSKH2, PYK2, QIK, QSK, RAF1, RAF1ps, RET, RHOK, RIPK1, RIPK2, RIPK3, RNAseL, ROCK1, ROCK2, RON, ROR1, ROR2, ROS, RSK1, RSK12, RSK2, RSK22, RSK3, RSK32, RSK4, RSK42, RSKL1, RSKL2, RYK, RYKps, SAKps, SBK, SCYL1, SCYL2, SCYL2ps, SCYL3, SGK, SgK050ps, SgK069, SgK071, SgK085, SgK110, SgK196, SGK2, SgK223, SgK269, SgK288, SGK3, SgK307, SgK384ps, SgK396, SgK424, SgK493, SgK494, SgK495, SgK496, SIK (e.g., SIK1, SIK2), skMLCK, SLK, Slob, smMLCK, SNRK, SPEG, SPEG2, SRC, SRM, SRPK1, SRPK2, SRPK2ps, SSTK, STK33, STK33ps, STLK3, STLK5, STLK6, STLK6ps1, STLK6-rs, SuRTK106, SYK, TAK1, TAO1, TAO2, TAO3, TBCK, TBK1, TEC, TESK1, TESK2, TGFbR1, TGFbR2, TIE1, TIE2, TLK1, TLKIps, TLK2, TLK2ps1, TLK2ps2, TNK1, Trad, Trb1, Trb2, Trb3, Trio, TRKA, TRKB, TRKC, TSSK1, TSSK2, TSSK3, TSSK4, TSSKps1, TSSKps2, TTBK1, TTBK2, TTK, TTN, TXK, TYK2, TYK22, TYRO3, TYRO3ps, ULK1, ULK2, ULK3, ULK4, VACAMKL, VRK1, VRK2, VRK3, VRK3ps, Weel, WeelB, WeelBps, Weelps1, Weelps2, Wnk1, Wnk2, Wnk3, Wnk4, YANK1, YANK2, YANK3, YES, YESps, YSK1, ZAK, ZAP70, ZC1/HGK, ZC2/TNIK, ZC3/MINK, and ZC4/NRK.

The term “histone” refers to highly alkaline proteins found in eukaryotic cell nuclei that package and order DNA into structural units called nucleosomes. They are the protein components of chromatin, acting as spools around which DNA winds, and play a role in gene regulation. In certain embodiments, the histone is histone H1 (e.g., histone H1F, histone H1H1). In certain embodiments, the histone is histone H2A (e.g., histone H2AF, histone H2A1, histone H2A2). In certain embodiments, the histone is histone H2B (e.g., histone H2BF, histone H2B1, histone H2B2). In certain embodiments, the histone is histone H3 (e.g., histone H3A1, histone H3A2, histone H3A3). In certain embodiments, the histone is histone H4 (e.g., histone H41, histone H44).

“Histone methyltransferases” or “HMTs” are histone-modifying enzymes that catalyze the transfer of one, two, or three methyl groups to lysine and/or arginine residues of histone proteins. HMTs modify histones at certain sites through methylation. Methylation of histones is of biological relevance because such methylation is an epigenetic modification of chromatin that determines gene expression, genomic stability, stem cell maturation, cell lineage development, genetic imprinting, DNA methylation, and/or cell mitosis. In certain embodiments, an HMT described herein is a histone-lysine N-methyltransferase. In certain embodiments, an HMT described herein is a histone-arginine N-methyltransferase. In certain embodiments, an HMT described herein is EZH1. In certain embodiments, an HMT described herein is EZH2. In certain embodiments, an HMT described herein is DOT1. In certain embodiments, an HMT described herein is G9a, GLP, MLL1, MLL2, MLL3, MLL4, NSD2, PRMT1, PRMT3, PRMT4, PRMT5, PRMT6, SET1b, SET7/9, SET8, SETMAR, SMYD2, SUV39H1, or SUV39H2.

The term “bromodomain” refers to a protein domain that recognizes acetylated lysine residues such as those on the N-terminal tails of histones. In certain embodiments, a bromodomain of a BET protein comprises about 110 amino acids and shares a conserved fold comprising a left-handed bundle of four alpha helices linked by diverse loop regions that interact with chromatin. In certain embodiments, the bromodomain is ASH1L (GenBank ID: gi|8922081), ATAD2 (GenBank ID: gi|24497618), BAZ2B (GenBank ID: gi|7304923), BRD1 (GenBank ID: gi|11321642), BRD2(1) (GenBank ID: gi|4826806), BRD2(2) (GenBank ID: gi|4826806), BRD3(1) (GenBank ID: gi|11067749), BRD3(2) (GenBank ID: gi|11067749), BRD4(1) (GenBank ID: gi|19718731), BRD4(2) (GenBank ID: gi|19718731), BRD9 (GenBank ID: gi|57770383), BRDT(1) (GenBank ID: gi|46399198), BRPF1 (GenBank ID: gi|51173720), CECR2 (GenBank ID: gi|148612882), CREBBP (GenBank ID: gi|4758056), EP300 (GenBank ID: gi|50345997), FALZ (GenBank ID: gi|38788274), GCN5L2 (GenBank ID: gi|10835101), KIAA1240 (GenBank ID: gi|51460532), LOC93349 (GenBank ID: gi|134133279), PB1(1) (GenBank ID: gi|30794372), PB1(2) (GenBank ID: gi|30794372), PB1(3) (GenBank ID: gi|30794372), PB1(5) (GenBank ID: gi|30794372), PB1(6) (GenBank ID: gi|30794372), PCAF (GenBank ID: gi|40805843), PHIP(2) (GenBank ID: gi|34996489), SMARCA2 (GenBank ID: gi|48255900), SMARCA4 (GenBank ID: gi|21071056), SP140 (GenBank ID: gi|52487219), TAF1(1) (GenBank ID: gi|20357585), TAF1(2) (GenBank ID: gi|20357585), TAF1L(1) (GenBank ID: gi|24429572), TAF1L(2) (GenBank ID: gi|24429572), TIF1 (GenBank ID: gi|14971415), TRIM28 (GenBank ID: gi|5032179), or WDR9(2) (GenBank ID: gi|16445436).

The term “bromodomain-containing protein,” “bromodomain protein,” or “BET protein” refers to a protein, whether wild-type or mutant, natural or synthetic, truncated or complete, or a variant thereof, that possesses the minimum amino acid sequence sufficient for a functional bromodomain capable of mediating molecular recognition of acetyl-lysine of acetylated lysine residues on a second protein (e.g., a histone), such as on the tails of histones.

The term “BRD4” or “Brd4” refers to Bromodomain-containing protein 4 that in humans is encoded by the BRD4 gene. BDR4 is a member of the BET (bromodomain and extra terminal domain) family, along with BRD2, BRD3, and BRDT. BRD4, similar to its BET family members, contains two bromodomains that recognize acetylated lysine residues. An increase in Brd4 expression leads to increased P-TEFb-dependent phosphorylation of RNA polymerase II (RNAPII) CTD and stimulation of transcription in vivo. Conversely, a reduction in Brd4 expression by siRNA reduced CTD phosphorylation and transcription, revealing that Brd4 is a positive regulatory component of P-TEFb. In chromatin immunoprecipitation (ChIP) assays, the recruitment of P-TEFb to a promoter was dependent on Brd4 and was enhanced by an increase in chromatin acetylation. Together, P-TEFb alternately interacts with Brd4 and the inhibitory subunit to maintain functional equilibrium in the cell.

The term “FKBP” refers to proteins that have prolyl isomerase activity. FKBPs have been identified in many eukaryotes as protein folding chaperones for proteins containing proline residues. FKBPs belong to the immunophilin family. Cytosolic signaling protein FKBP12 is notable in humans for binding the immunosuppressant molecule tacrolimus. FKBP12 contains a PPIase core domain, which is found in many FKBPs, and occurs in many species and is essential to mammals. FKBP12 is implicated in various diseases.

The term “aryl hydrocarbon receptor”, “AhR”, “AHR”, “ahr”, or “ahR” is a transcription factor that regulates gene expression. The aryl hydrocarbon receptor is a cytosolic transcription factor that exist bound to co-chaperones in the resting state. Upon ligand binding, the co-chaperones dissociate, allowing AHR to translocate to the nucleus, dimerize, and alter transcription of target genes. AHR play a role in regulating metabolism enzymes, immunity, stem cell maintenance, and cellular differentiation.

A “nuclear protein” is a protein found in a cell nucleus. As used herein, the term “nuclear receptor” relates to a class of proteins found within cells that are responsible for sensing steroid and thyroid hormones and certain other molecules. In response, these receptors work with other proteins to regulate the expression of specific genes, thereby controlling the development, homeostasis, and metabolism of the organism. Since the expression of a large number of genes is regulated by nuclear receptors, ligands that activate these receptors can have profound effects on the organism.

“Histone deacetylase” or “HDAC” are a class of enzymes that remove acetyl groups from a histone, which allows histones to bind DNA and inhibit gene transcription. Examples of HDACs include, but are not limited to, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, and SIRT7.

“Lysine methyltransferases” are enzymes that catalyze the transfer of methyl groups from S-adenosylmethionine (SAM) to the lysine residues on histones. Lysine methyltransferases belong to the histone methyltransferase group of enzymes.

A “transcription factor” is a type of protein that is involved in the process of transcribing DNA into RNA. Transcription factors can work independently or with other proteins in a complex to either stimulate or repress transcription. Transcription factors contain at least one DNA-binding domain that give them the ability to bind to specific sequences of DNA. Other proteins such as coactivators, chromatin remodelers, histone acetyltransferases, histone deacetylases, kinases, and methylases are also essential to gene regulation, but lack DNA-binding domains, and therefore are not transcription factors. Non-limiting examples of transcription factors include TFIIA, TFIIB, TFIID, TFIIE, TFIIF, TFIIH, SP1, AP-1, C/EBP, ATF/CREB, NF1, CCAAT, GATA, HNF, PIT-1, MyOD, Myf5, Hox, Winged Helix, SREBP, p53, Mef2, STAT, R-SMAD, NF-κB, SMARCA4, SMARCA2, TRIM24, and TUBBY.

As used herein, the term “SMARCA4” relates to transcription activator BRG1 also known as ATP-dependent helicase. SMARCA4 is a protein that in humans is encoded by the SMARCA4 gene. Mutations are linked to lung cancer cell lines. BRG1 plays a role in the control of retinoic acid and glucocorticoid-induced cell differentiation in lung cancer and other tumors.

A “hormone receptor” is a receptor that binds to a specific hormone and are a wide family of proteins made up of receptors for thyroid and steroid hormones, and other various ligands. There are two main classes of hormone receptors: trans membrane receptors and intracellular or nuclear receptors. Examples include androgen receptors, calcitriol receptors, corticotropin-releasing hormone receptor 1, corticotropin releasing hormone receptor 2, estrogen receptors, follicle-stimulating hormone receptors, glucagon receptors, gonadotropin receptors, gonadotropin-releasing hormone receptors, growth hormone receptors, insulin receptor, luteinizing hormone, progesterone receptors, retinoid receptors, somatostatin receptors, thyroid hormone receptors, and thyrotropin receptors.

The term “cyclimid” refers to a class of CRBN ligands inspired by the C-terminal cyclic imide degron, which for example, serve as E3 ligase binders in the development of PROTACs, and comprise C-terminal cyclic imide degron. Cyclic imides include moieties of the formula:

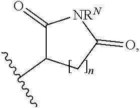

wherein n is 1, 2, or 3. For example, cyclimids disclosed herein include XcQ (cQ stands for cyclized glutamine (i.e., cyclized to form a glutarimide)) and XcN (cN stands for cyclized asparagine (i.e., cyclized to form an aspartimide)), wherein XcN is of the formula:

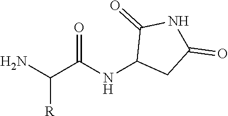

and XcQ is of the formula:

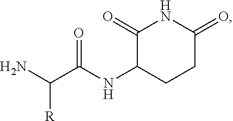

wherein X represents the one letter code of an amino acid and R is the corresponding amino acid side chain. When a prefix is present (e.g., Boc in Boc-FcQ), then the group is attached through the terminal amine (—NH₂) as drawn above (e.g., Boc-FcQ has the following structure:

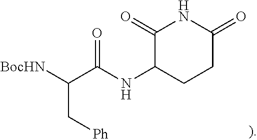 [SMILES: CC(C)(C)OC(=O)NC(Cc1ccccc1)C(=O)NC1CCC(=O)NC1=O]

The term “binder” refers to a compound that binds to the target. The term “target” refers to protein, polypeptide, molecule (e.g., signaling molecule), receptor, enzyme, etc. of interest. The term binder is used herein to describe a compound (e.g., small molecule, protein, peptide, sugar), which binds to a target (e.g., protein, polypeptide, molecule (e.g., signaling molecule), receptor, enzyme, etc.) of interest and places/presents that protein or polypeptide in proximity to a ubiquitin ligase such that ubiquitination of the protein or polypeptide by ubiquitin ligase and subsequent degradation may occur. Any compound which can bind to the target moiety is a binder. Any compound or construct that be acted on or be degraded by a ubiquitin ligase, is a target. In some embodiments the target is a protein. In some embodiments, the target proteins may include, for example, structural proteins, receptors, enzymes, cell surface proteins, proteins pertinent to the integrated function of a cell, including proteins involved in catalytic activity (e.g., aromatase activity, motor activity, helicase activity, metabolic processes (anabolism and catabolism), antioxidant activity, proteolysis, biosynthesis, oxidoreductase activity, transferase activity, hydrolase activity, lyase activity, isomerase activity, ligase activity); proteins with kinase activity, enzyme regulator activity, signal transducer activity, structural molecule activity, proteins with binding activity (e.g., bind to a protein, lipid, carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, or translation regulator activity. Proteins of interest can include proteins from prokaryotes and eukaryotes, including humans, as targets for drug therapy; from other animals, including domesticated animals; proteins from microbials for the determination of targets for antibiotics; proteins from other antimicrobials and plants; and even proteins from viruses, among numerous other sources of proteins. Non-limiting examples of small molecule target protein binding moieties include Hsp90 inhibitors, kinase inhibitors, MDM2 inhibitors, compounds targeting BET Bromodomain-containing proteins, HDAC inhibitors, lysine methyltransferase inhibitors, angiogenesis inhibitors, immunosuppressive compounds, and compounds targeting the aryl hydrocarbon receptor (AHR), among numerous other target protein binding moieties.

The term “small molecule” refers to molecules, whether naturally-occurring or artificially created (e.g., via chemical synthesis) that have a relatively low molecular weight. In some embodiments, the small molecule is found in the body. Typically, a small molecule is an organic compound (e.g., it contains carbon). A small molecule may be an inorganic compound in some embodiments. The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, at least about 1,000 g/mol, at least about 1,100 g/mol, at least about 1,200 g/mol, at least about 1,300 g/mol, at least about 1,400 g/mol, at least about 1,500 g/mol, at least about 2,000 g/mol, at least about 2,500 g/mol, or at least about 3,000 g/mol. Combinations of the above ranges (e.g., at least about 200 g/mol and not more than about 500 g/mol) are also possible.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher than normal risk of becoming abnormal or unhealthy, which may need to be prevented. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for binding a target (e.g., a protein (e.g., a bromodomain or bromodomain-containing protein (e.g., BRD4), a HMT, a kinase (e.g., a tyrosine kinase, a serine/threonine kinase, a cyclin dependent kinase (e.g., CDK4, CDK6), or a leucine-rich repeat kinase), a cytosolic signaling protein (e.g., FKBP12), a nuclear protein, a histone deacetylase, a lysine methyltransferase, a protein regulating angiogenesis, a protein regulating immune response, an aryl hydrocarbon receptor (AHR), a hormone receptor (e.g., an estrogen receptor, an androgen receptor, a glucocorticoid receptor), or a transcription factor (e.g., SMARCA4, SMARCA2, or TRIM24)). In certain embodiments, a therapeutically effective amount is an amount sufficient for treating a proliferative disease (e.g., cancer). In certain embodiments, a therapeutically effective amount is an amount sufficient for binding a target protein (e.g., a bromodomain or bromodomain-containing protein (e.g., BRD4), a HMT, a kinase (e.g., a tyrosine kinase, a serine/threonine kinase, a cyclin dependent kinase (e.g., CDK4, CDK6), or a leucine-rich repeat kinase), a cytosolic signaling protein (e.g., FKBP12), a nuclear protein, a histone deacetylase, a lysine methyltransferase, a protein regulating angiogenesis, a protein regulating immune response, an aryl hydrocarbon receptor (AHR), a hormone receptor (e.g., an estrogen receptor, an androgen receptor, a glucocorticoid receptor), or a transcription factor (e.g., SMARCA4, SMARCA2, or TRIM24)) and/or inducing the degradation of the target (e.g., a protein (e.g., a bromodomain or bromodomain-containing protein (e.g., BRD4), a HMT, a kinase (e.g., a tyrosine kinase, a serine/threonine kinase, a cyclin dependent kinase (e.g., CDK4, CDK6), or a leucine-rich repeat kinase), a cytosolic signaling protein (e.g., FKBP12), a nuclear protein, a histone deacetylase, a lysine methyltransferase, a protein regulating angiogenesis, a protein regulating immune response, an aryl hydrocarbon receptor (AHR), a hormone receptor (e.g., an estrogen receptor, an androgen receptor, a glucocorticoid receptor), or a transcription factor (e.g., SMARCA4, SMARCA2, or TRIM24)).

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for binding a target (e.g., a bromodomain or bromodomain-containing protein (e.g., BRD4), a HMT, a kinase (e.g., a tyrosine kinase, a serine/threonine kinase, a cyclin dependent kinase (e.g., CDK4, CDK6), or a leucine-rich repeat kinase), a cytosolic signaling protein (e.g., FKBP12), a nuclear protein, a histone deacetylase, a lysine methyltransferase, a protein regulating angiogenesis, a protein regulating immune response, an aryl hydrocarbon receptor (AHR), a hormone receptor (e.g., an estrogen receptor, an androgen receptor, a glucocorticoid receptor), or a transcription factor (e.g., SMARCA4, SMARCA2, or TRIM24)) and/or inducing the degradation of the target protein (e.g., a bromodomain or bromodomain-containing protein (e.g., BRD4), a HMT, a kinase (e.g., a tyrosine kinase, a serine/threonine kinase, a cyclin dependent kinase (e.g., CDK4, CDK6), or a leucine-rich repeat kinase), a cytosolic signaling protein (e.g., FKBP12), a nuclear protein, a histone deacetylase, a lysine methyltransferase, a protein regulating angiogenesis, a protein regulating immune response, an aryl hydrocarbon receptor (AHR), a hormone receptor (e.g., an estrogen receptor, an androgen receptor, a glucocorticoid receptor), or a transcription factor (e.g., SMARCA4, SMARCA2, or TRIM24)). In certain embodiments, a prophylactically effective amount is an amount sufficient for treating a disease (e.g., proliferative disease, cancer, benign neoplasms, inflammatory disease, autoimmune disease).

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

As used herein the term “inhibit” or “inhibition” in the context of enzymes, for example, in the context of BRD4, CDK4, CDK6, or FKBP, refers to a reduction in the activity of the enzyme. In some embodiments, the term refers to a reduction of the level of enzyme activity, e.g., BRD4, CDK4, CDK6, or FKBP activity, to a level that is statistically significantly lower than an initial level, which may, for example, be a baseline level of enzyme activity. In some embodiments, the term refers to a reduction of the level of enzyme activity, e.g., BRD4, CDK4, CDK6, or FKBP activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

When a compound, pharmaceutical composition, method, use, or kit is referred to as “selectively,” “specifically,” or “competitively” inhibiting the target that B binds to, the compound, pharmaceutical composition, method, use, or kit inhibits the target to a greater extent (e.g., not less than 2-fold, not less than 5-fold, not less than 10-fold, not less than 30-fold, not less than 100-fold, not less than 1,000-fold, or not less than 10,000-fold; and/or: not more than 2-fold, not more than 5-fold, not more than 10-fold, not more than 30-fold, not more than 100-fold, not more than 1,000-fold, or not more than 10,000-fold) than inhibiting other proteins.

When a compound, pharmaceutical composition, method, use, or kit is referred to as “selectively,” “specifically,” or “competitively” inhibiting BRD4, CDK4, CDK6, or FKBP, the compound, pharmaceutical composition, method, use, or kit inhibits BRD4, CDK4, CDK6, or FKBP to a greater extent (e.g., not less than 2-fold, not less than 5-fold, not less than 10-fold, not less than 30-fold, not less than 100-fold, not less than 1,000-fold, or not less than 10,000-fold; and/or: not more than 2-fold, not more than 5-fold, not more than 10-fold, not more than 30-fold, not more than 100-fold, not more than 1,000-fold, or not more than 10,000-fold) than inhibiting other proteins.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, hemolytic autoimmune anemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenia purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischemic colitis, diversion colitis, Behcet's syndrome, indeterminate colitis) and inflammatory bowel syndrome (IBS)), lupus, multiple sclerosis, morphea, myasthenia gravis, myocardial ischemia, nephrotic syndrome, pemphigus vulgaris, pernicious anemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, scleroderma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosus, rheumatoid arthritis, psoriatic arthritis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, hantavirus, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMOL), polycythemia vera, lymphoma, acute lymphocytic leukemia (ALL), chronic lymphocytic leukemia, Wilms tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenia purpura and disseminated intravascular coagulation, infections by parasites such as Plasmodium, chemical injuries from, e.g., lead poisoning, and hypersplenism.

The terms “genetic disease” or “genetic related disease” refer to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; Lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating.

In certain embodiments, the painful condition is neuropathic pain. The term “neuropathic pain” refers to pain resulting from injury to a nerve. Neuropathic pain is distinguished from nociceptive pain, which is the pain caused by acute tissue injury involving small cutaneous nerves or small nerves in muscle or connective tissue. Neuropathic pain typically is long-lasting or chronic and often develops days or months following an initial acute tissue injury. Neuropathic pain can involve persistent, spontaneous pain as well as allodynia, which is a painful response to a stimulus that normally is not painful. Neuropathic pain also can be characterized by hyperalgesia, in which there is an accentuated response to a painful stimulus that usually is trivial, such as a pin prick. Neuropathic pain conditions can develop following neuronal injury and the resulting pain may persist for months or years, even after the original injury has healed. Neuronal injury may occur in the peripheral nerves, dorsal roots, spinal cord or certain regions in the brain. Neuropathic pain conditions include, but are not limited to, diabetic neuropathy (e.g., peripheral diabetic neuropathy); sciatica; non-specific lower back pain; multiple sclerosis pain; carpal tunnel syndrome, fibromyalgia; HIV-related neuropathy; neuralgia (e.g., post-herpetic neuralgia, trigeminal neuralgia); pain resulting from physical trauma (e.g., amputation; surgery, invasive medical procedures, toxins, burns, infection), pain resulting from cancer or chemotherapy (e.g., chemotherapy-induced pain such as chemotherapy-induced peripheral neuropathy), and pain resulting from an inflammatory condition (e.g., a chronic inflammatory condition). Neuropathic pain can result from a peripheral nerve disorder such as neuroma; nerve compression; nerve crush, nerve stretch or incomplete nerve transection; mononeuropathy or polyneuropathy. Neuropathic pain can also result from a disorder such as dorsal root ganglion compression; inflammation of the spinal cord; contusion, tumor or hemisection of the spinal cord; tumors of the brainstem, thalamus or cortex; or trauma to the brainstem, thalamus or cortex. The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia), or an absence of or deficit in selective sensory pathways (hypoalgesia).

In certain embodiments, the painful condition is non-inflammatory pain. The types of non-inflammatory pain include, without limitation, peripheral neuropathic pain (e.g., pain caused by a lesion or dysfunction in the peripheral nervous system), central pain (e.g., pain caused by a lesion or dysfunction of the central nervous system), deafferentation pain (e.g., pain due to loss of sensory input to the central nervous system), chronic nociceptive pain (e.g., certain types of cancer pain), noxious stimulus of nociceptive receptors (e.g., pain felt in response to tissue damage or impending tissue damage), phantom pain (e.g., pain felt in a part of the body that no longer exists, such as a limb that has been amputated), pain felt by psychiatric subjects (e.g., pain where no physical cause may exist), and wandering pain (e.g., wherein the pain repeatedly changes location in the body).

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY, or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

The terms “infectious disease” and “infectious disorder” refer to diseases and disorders caused by microorganisms, for example bacteria, viruses, fungi, or parasite. Exemplary infectious diseases include, but are not limited to, Acute Flaccid Myelitis (AFM), anaplasmosis, anthrax, babesiosis, botulism, brucellosis, campylobacteriosis, carbapenem-resistant infection (CRE/CRPA), chancroid, chikungunya virus infection, chlamydia, ciguatera (Harmful Algae Blooms (HABs)), Clostridium difficile infection, Clostridium perfringens (epsilon toxin), coccidioidomycosis fungal infection (valley fever), COVID-19, Creutzfeldt-Jakob disease, transmissible spongiform encephalopathy (CJD), cryptosporidiosis, cyclosporiasis, dengue fever, diphtheria, E. coli infection, Shiga toxin-producing (STEC), eastern equine encephalitis, Ebola, ehrlichiosis, encephalitis, arboviral or parainfectious, enterovirus infection, non-polio enterovirus, enterovirus infection, D68 (EV-D68), giardiasis (giardia), glanders, gonococcal infection (gonorrhea), granuloma inguinale, haemophilus influenza disease, hantavirus pulmonary syndrome, hemolytic uremic syndrome, hepatitis A, hepatitis B, hepatitis C, hepatitis D, hepatitis E, herpes, herpes zoster, shingles, histoplasmosis infection, human immunodeficiency virus, AIDS, human papillomavirus, influenza, lead poisoning, legionellosis (Legionnaires Disease), leprosy (Hansens Disease), leptospirosis, listeriosis (Listeria), Lyme disease, lymphogranuloma venereum infection (LGV), malaria, measles, melioidosis, meningitis, viral (Meningitis, viral), meningococcal disease, bacterial (Meningitis, bacterial), middle east respiratory syndrome coronavirus (MERS-COV), multisystem inflammatory syndrome in children (MIS-C), mumps, norovirus, paralytic shellfish poisoning, pediculosis, lice, pelvic inflammatory disease, pertussis, plague (bubonic, septicemic, pneumonic), pneumonia, poliomyelitis (Polio), Powassan virus, psittacosis (Parrot Fever), pthiriasis, pustular rash diseases (small pox, monkeypox, cowpox), Q-Fever, rabies, ricin poisoning, rickettsiosis (Rocky Mountain Spotted Fever), rubella, salmonella, scabies, scombroid, septic shock (Sepsis), severe acute respiratory syndrome (SARS), shigellosis gastroenteritis (shigella), smallpox, staphylococcal infections (MRSA, staph food poisoning, VISA, VRSA), streptococcal Disease (strep A, strep B), streptococcal toxic-shock syndrome, syphilis, tetanus infection, trichomoniasis, trichonosis infection, tuberculosis, tularemia, typhoid fever, typhus, vaginosis, vaping-associated lung injury, varicella, Vibrio cholerae (cholera), vibriosis (vibrio), viral hemorrhagic fever (Ebola, Lassa, Marburg), West Nile Virus, yellow fever, yersenia, or zika virus infection (Zika).

The terms “cardiovascular disease” and “cardiovascular disorder” refer to diseases and disorders that affect the heart or blood vessels. Exemplary cardiovascular diseases include, but are not limited to, angina, arrhythmia, congenital heart disease, coronary artery disease, heart attack, heart failure, dilated cardiomyopathy, hypertrophic cardiomyopathy, mitral regurgitation, mitral valve prolapse, pulmonary stenosis, aortic stenosis, atrial fibrillation, rheumatic heart disease, radiation heart disease, peripheral artery disease, aneurysm, atherosclerosis, renal artery disease, Raynaud's disease, peripheral venous disease, ischemic stroke, venous blood clots, blood clotting disorders, or Buerger's disease.

The terms “cerebrovascular disease” and “cerebrovascular disorder” refer to diseases and disorders that affect blood flow and blood vessels in the brain. The diseases and disorders may be due to stenosis, thrombosis, embolism, or hemorrhage. Exemplary cerebrovascular diseases include, but are not limited to, aneurysm, arteriovenous malformation (AVM), cerebral cavernous malformation (CCM), arteriovenous fistula (AVF), carotid-cavernous fistula, carotid stenosis, transient ischemic attack (TIA), or stroke.

The terms “pulmonary disease” or “pulmonary disorder” refer to diseases and disorders relating to the lungs. Exemplary pulmonary diseases include, but are not limited to, asbestosis, asthma, bronchiectasis, bronchitis, chronic cough, chronic obstructive pulmonary disease (COPD), common cold, COVID-19, croup, cystic fibrosis, hantavirus, influenza, idiopathic pulmonary fibrosis, lung cancer, pandemic flu, pertussis, pleurisy, pneumonia, pulmonary edema, pulmonary Embolism, pulmonary fibrosis, pulmonary Hypertension, respiratory syncytial virus (RSV), sarcoidosis, sleep apnea, spirometry, sudden infant death syndrome (SIDS), or tuberculosis.

The terms “dermatological disease” or “dermatological disorder” refers to diseases and disorders relating to the skin. Exemplary dermatological diseases include, but are not limited to, acanthoma fissuratum, acanthosis nigricans, accessory tragus, acne, acne excoriée, acne keloidalis nuchae, acquired digital fibrokeratoma, acrochordons, acrodermatitis enteropathica, acropustulosis of infancy, actinic cheilitis, actinic keratosis, actinic purpura, dolorosa (Dercum's disease), albinism, alkaptonuria, allergic contact dermatitis, alopecia areata, alopecia mucinosa, androgenetic alopecia, anetoderma, angioedema, angiofibroma, angiokeratoma, angiomas, angular cheilitis, aphthous ulcer, aplasia cutis congenita, ashy dermatosis, asteatotic eczema, atopic dermatitis, atrophie blanche, atrophoderma of pasini and pierini, atypical moles, balanitis, basal cell carcinoma, basal cell nevus syndrome, Becker's nevus, bee and wasp stings, black hairy tongue, Blaschko's lines, blue nevus, boils, Bowen's disease, Bowenoid papulosis, brachioradial pruritus, bullous pemphigoid, Buruli ulcer, calcipotriene, canker sore, capsaicin, carbuncle, cellulitis, central centrifugal cicatricial alopecia, chancroid, cherry angioma, chicken pox, chondrodermatitis nodularis helicis, condyloma acuminata, confluent and reticulated papillomatosis, congenital adrenal hyperplasia, contact dermatitis, Cowden syndrome, cutaneous T cell lymphoma, cutis marmorata, cysts, dandruff, Darier disease, dermal fillers, dermatitis herpetiformis, dermatofibroma, dermatofibrosarcoma protuberans, dermatographism, dermatomyositis, dermatosis papulosa nigra, diaper dermatitis, digital mucous cyst, discoid lupus erythematosus, disseminated superficial actinic porokeratosis, DRESS syndrome, dry skin, dyshidrotic dermatitis, dysplastic moles, eczema, Ehlers-Danlos syndrome, elastosis perforans serpiginosa, epidermal cyst, epidermal nevus, epidermolysis bullosa, epidermolysis bullosa acquisita, erosive pustular dermatosis, erysipelas, erythema ab igne, erythema annulare centrifugum, erythema dyschromicum perstans, erythema infectiosum, erythema multiforme, erythema nodosum, erythema toxicum neonatorum, erythrasma, erythromelalgia, etanercept, exanthem subitum, Fabry disease, Favre-Racouchot syndrome, female pattern hair loss, fifth disease (also referred to as erythema infectiosum), fire ant bites, fish tank granuloma, flea bites, focal dermal hypoplasia, folliculitis, Fordyce spots, Fox-Fordyce disease, frostbite, fungal infections, furuncle, geographic tongue, Gianotti-Crosti syndrome, glomus tumor, Gorlin syndrome, granuloma annulare, granuloma inguinale, green nail syndrome, Grover's disease, habit tic nail deformity, Hailey-Hailey disease, hair loss, hair removal, hair transplantation, halo moles, hand rashes, hand foot and mouth disease, head lice, hemangiomas, Henoch-Schonlein purpura, hereditary hemorrhagic telangiectasia, herpes, herpes zoster, hidradenitis suppurativa, hidrocystoma, hirsutism, hives, hot tub folliculitis, hyperhidrosis, hyperpigmentation, hypersensitivity vasculitis, hypomelanosis of ito, ichthyosis, idiopathic guttate hypomelanosis, impetigo, incontinentia pigmenti, ingenol mebutate, intertrigo, itching, Jessner lymphocytic infiltrate, jiggers, juvenile plantar dermatosis, juvenile xanthogranuloma, Kaposi's sarcoma, Kawasaki's disease, keloids and hypertrophic scars, keratoacanthoma, keratosis follicularis spinulosa decalvans, keratosis pilaris, Kyrle's disease, leiomyoma, leishmaniasis, lentigines, lentigo maligna, leprosy, leukocytoclastic vasculitis, leukoplakia, lice, lichen amyloidosis, lichen nitidus, lichen planus, lichen sclerosus, lichen simplex chronicus, lichen spinulosus, lichen striatus, linear IgA bullous dermatosis, lipodermatosclerosis, lipoma, loose anagen syndrome, Lyme disease, lymphangioma circumscriptum, lymphogranuloma venereum, majocchi granuloma, mastocytoma, mastocytosis, measles, melanoma, acral lentiginous, melanoma in situ, melanonychia, melasma, Melkersson-Rosenthal syndrome, meralgia paresthetica, Merkel cell carcinoma, metastatic skin cancer, methotrexate, milia, miliaria, moles, molluscum contagiosum, Mongolian spot, monilethrix, morphea, mucocele, muir-torre syndrome, mycophenolate mofetil, mycosis fungoides, myiasis, nail fungus, necrobiosis lipoidica diabeticorum, neurofibromatosis, nevoid basal cell carcinoma syndrome, nevus achromicus, nevus anemicus, nevus flammeus, nevus sebaceus, nevus spilus, nevus of ota and ito, notalgia paresthetica, nummular eczema, ochronosis, onycholysis, onychomycosis, onychophagia, orange palpebral spots, paederus dermatitis, Paget's disease, palmoplantar pustulosis, panniculitis, parapsoriasis, paronychia nail infection, pediculosis, pellagra, pemphigus, pemphigoid gestationis, perioral dermatitis, perleche, Peutz-Jeghers syndrome, phytophotodermatitis, pilar cyst, pilomatricoma, pimecrolimus, pitted keratolysis, pityriasis alba, pityriasis lichenoides, pityriasis rosea, pityriasis rubra pilaris, pityrosporum folliculitis, poikiloderma of civatte, poison ivy dermatitis, polymorphous light eruption, porokeratosis of mibelli, porphyria cutanea tarda, postherpetic neuralgia, pressure ulcers, progressive pigmentary purpura, prurigo nodularis, prurigo pigmentosa, pruritic urticarial papules and plaques of pregnancy, pseudofolliculitis barbae, pseudoxanthoma elasticum, psoriasis, punctate palmoplantar keratoderma, pyoderma gangrenosum, pyogenic granuloma, red scrotum syndrome, rheumatoid nodules, rocky mountain spotted fever, rosacea, roseola infantum, sarcoidosis, scabies, scarlet fever, Schamberg's disease, scleroderma, sebaceous cyst, sebaceous hyperplasia, seborrheic dermatitis, seborrheic keratoses, shingles, skin tags, spider angioma, spider bites, spitz nevus, sporotrichosis, squamous cell carcinoma, staphylococcal scalded skin syndrome, stasis dermatitis, steatocystoma multiplex, Stevens Johnson syndrome, stretch marks, striae, Sturge-Weber syndrome, subacute cutaneous lupus erythematosus, subungual hematoma, sweet's syndrome, swimmer's itch, syphilis, syringoma, systemic lupus erythematosus, systemic sclerosis, elangiectasia macularis eruptiva perstans, telogen effluvium hair loss, tinea, tinea incognito, tinea versicolor, toxic epidermal necrolysis, transient neonatal pustular melanosis, tretinoin, trichilemmal cyst, trichotillomania, tuberous sclerosis, twenty nail dystrophy, urticaria, urticaria pigmentosum, varicella, vitiligo, warts, washboard nail, xanthelasma, xeroderma pigmentosum, xerosis, or xerotic eczema.

The terms “bone diseases” and “bone disorders” refer to diseases and disorders affecting bones. Exemplary bone diseases include, but are not limited to, bone spurs, bone tumor, chondroblastoma, chondromyxoid fibroma, enchondroma, extra-abdominal desmoid tumors, fibrous dysplasia, hypophosphatasia, Klippel-Feil Syndrome, osteochondritis dissecans (OCD), osteochondroma, osteoporosis, osteopetrosis, osteonecrosis, osteitis deformans, osteogenesis imperfecta, or osteoid osteoma.

The terms “hormonal diseases” and “hormonal disorders” refer to diseases and disorders regulated or related to hormones and/or the endocrine system. Exemplary hormonal diseases include, but are not limited to, acromegaly, Addison's Disease, adrenal cancer, adrenal disorders, anaplastic thyroid cancer, Cushing's Syndrome, De Quervain's thyroiditis, diabetes, follicular thyroid cancer, gestational diabetes, goiters, Graves' Disease, growth disorders, growth hormone deficiency, Hashimoto's thyroiditis, heart disease, Hurthle cell thyroid cancer, hyperglycemia, hyperparathyroidism, hyperthyroidism, hypoglycemia, hypoparathyroidism, hypothyroidism, low testosterone, medullary thyroid cancer, MEN 1, MEN 2A, MEN 2B, menopause, metabolic syndrome, obesity, osteoporosis, papillary thyroid cancer, parathyroid diseases, pheochromocytoma, pituitary disorders, pituitary tumors, polycystic ovary syndrome, prediabetes, reproduction, silent thyroiditis, thyroid cancer, thyroid diseases, thyroid nodules, thyroiditis, turner syndrome, Type 1 diabetes, or Type 2 diabetes.

Reference to “a compound of the disclosure,” “a compound provided herein,” “a compound disclosed herein,” “a compound described herein,” and the like, means any compound disclosed herein; specifically, a compound of Formula (I′) or (I), or any subgenus or species thereof, or any pharmaceutically acceptable salt, stereoisomer, tautomer, isotopically labeled derivative, solvate, hydrate, polymorph, co-crystal, or prodrug thereof. In the various aspects and embodiments disclosed herein, express reference to a compound of Formula (I′) or (I) is understood to alternatively refer to a compound of any disclosed subgenus or species thereof, or a pharmaceutically acceptable salt, stereoisomer, tautomer, isotopically labeled derivative, solvate, hydrate, polymorph, co-crystal, or prodrug thereof. In some embodiments a compound of Formula (I′) or (I) is a compound of Formula (I′) or (I), or pharmaceutically acceptable salt, stereoisomer, tautomer, isotopically labeled derivative, solvate, hydrate, polymorph, co-crystal, or prodrug thereof. In some embodiments a compound of Formula (I′) or (I) is a compound of Formula (I′) or (I), or pharmaceutically acceptable salt, stereoisomer, tautomer, solvate, hydrate, polymorph, or co-crystal thereof. In some embodiments a compound of Formula (I′) or (I) is a compound of Formula (I′) or (I), or pharmaceutically acceptable salt, stereoisomer, or tautomer thereof. In some embodiments a compound of Formula (I′) or (I) is a compound of Formula (I′) or (I), or pharmaceutically acceptable salt or tautomer thereof. In some embodiments a compound of Formula (I′) or (I) is the free base of a compound of Formula (I′) or (I).

These and other exemplary substituents are described in more detail in the Detailed Description, Examples, Figures, and Claims. The invention is not limited in any manner by the above exemplary listing of substituents.

### DETAILED DESCRIPTION

Before the disclosed compounds, compositions, kits, methods, and uses are described in more detail, it should be understood that the aspects described herein are not limited to specific embodiments, methods, uses, or configurations, and as such can, of course, vary. It is also to be understood that the terminology used herein is for the purpose of describing particular aspects only and, unless specifically defined herein, is not intended to be limiting.

Provided herein is a targeted protein degradation strategy used to discover that C-terminal cyclic imides are versatile and flexible degrons recognized by the thalidomide binding domain of CRBN. Targeted protein degradation strategies typically use small molecules as mimetics of the degrons of E3 ligases to redirect substrate recognition to a desired target protein.^{29,5} Here, this strategy was implemented in the reverse direction, leveraging small molecule degraders to identify biologically relevant structural motifs capable of functionally engaging CRBN within a cell. The data presented herein demonstrates that dipeptides with variable amino acids at the N−1 position conjugated to a C-terminal glutarimide or aspartimide are the minimal degrons to functionally engage CRBN in the degradation of BRD4. Measurement of the ternary complex in vitro and in cells revealed that the dipeptide degraders are ligands for CRBN. Deeper investigation of one degron, FcQ, revealed its ability to promote degradation of additional substrates (e.g., FKBP12 and CDK6) when incorporated into different small molecule degraders. Incorporation of the glutarimide and aspartimide degrons into bifunctional small molecule degraders (e.g., PROTACs (i.e., PROTACs comprising either glutarimide or aspartimide with one of JQ1 or dFKBP)) induced selective ubiquitylation by CRBN in vitro and degradation in cells. Also, endogenous proteins bearing the C-terminal cyclic imide degron are globally up-regulated upon CRBN knockout or the inhibition of the thalidomide-binding domain of CRBN, indicating those proteins are broadly regulated by CRBN. These properties are prototypical for a degron, and analogous to other degrons that are generated post-translationally or constitutively found at the N- and C-termini of proteins.⁴⁶

Provided herein is the insight that thalidomide and its derivatives are mimics of a C-terminal cyclic imide degron for CRBN enables exploitation of new chemical space for novel degrader design and mechanism of action studies. Mechanistically, the dipeptide Boc-FcQ does not mediate the same ternary complexes as the IMiDs, and the differences between CRBN binding (e.g., inhibition) and substrate degradation by the IMiDs may now be differentiable by the dipeptide degron. The dipeptide scaffold may further mitigate the potential for off-target degradation from the IMiDs when employed in bifunctional degraders that engage CRBN relative to IMiDs.^{7,47} The IMiDs additionally possess multiple biological activities including immunomodulatory functions that regulate signaling through NFκB, IRF4, and TNFα,⁴⁸ which may be compared with the dipeptides to shed light on mechanisms that are common to the glutarimide moiety or those that lead to substrate degradation promoted by the IMiDs.

The discovery that the conserved thalidomide binding domain of CRBN recognizes C-terminal cyclic imides for degradation also implies that these are previously underappreciated post-translational modifications (PTMs). Recognition and removal of these modified proteins by CRBN, together with the labile nature of C-terminal cyclic imides toward hydrolysis, has limited their ready observation in the proteome to date. Cyclic imides may form from the aging process and thus give rise to damaged proteins that are recognized and removed by CRBN. It was observed that there are cN and cQ modifications at several thousand sites on several hundred proteins in proteomics datasets from across human tissues, and verified the existence of these modifications in red blood cells (e.g., hemoglobin subunits alpha and beta in red blood cells), bovine eyes, HEK293T cells, and MM.1S cells. Further evaluation of these modified proteins and the role of the cyclic imide PTM may be accelerated by the development of specific enrichment and detection methods for C-terminal cyclic imides to visualize and map these PTMs. Additionally, a stronger understanding of the genesis of C-terminal cyclic imides will enable further definition of the physiological function of the thalidomide-binding domain of CRBN. The C-terminal cyclic imide degron may represent an overlooked form of protein damage that is generated adventitiously at susceptible asparagine and glutamine residues across the proteome, which CRBN recognizes and removes. This model is reminiscent of the cellular machinery that is conserved to protect organisms from another form of spontaneous protein damage, the isoaspartate PTM, which arises from spontaneous deamidation at asparagine residues and is particularly important in the brain (E. Kim et al., Proc Natl Acad Sci USA 94, 6132-6137 (1997)). In line with a protein damage model, it was observed that these modifications readily form on peptides in vitro in a pH-dependent manner, and the abundance of C-terminal cyclic imide-bearing peptides, as well as their downstream hydrolyzed products, largely increase when CRBN is knocked out or the thalidomide-binding domain of CRBN is inhibited by lenalidomide. These observations are congruent with a model where CRBN is conserved to regulate removal of these damaged proteins, thus preventing the undesired accumulation of damaged protein fragments. Alternatively, these modifications may form during an enzymatic process or a concerted protein splicing mechanism, such as that observed during intein excision, which may specifically generate these PTMs. The mechanism of formation of these modifications, and whether or not an enzymatic process promotes their formation, will lead to further investigation, as to date C-terminal aspartimide formation in mammals has primarily been observed during protein aging,^{21,49} and glutarimide formation has been observed sparingly from deamidation studies of long-lived proteins⁵⁰ and proposed as an intermediate in protein splicing.²⁵

As C-terminal cyclic imide sites and substrates become more clearly defined, the mechanistic implications of these PTMs and their role in protein regulation and cellular signaling, particularly in instances of intellectual disability caused by the loss of the thalidomide-binding domain of CRBN (J. J. Higgins et al., Neurology 63, 1927-1931 (2004)) will also be a major future direction of inquiry. Further investigation into the cyclic imide modifications will elucidate the substrates and mechanisms regulated by CRBN and the biology mediated by these PTMs beyond CRBN-dependent protein degradation.

### Compounds

Provided herein are compounds of Formula (I′) and Formula (I). In some embodiments, a compound of Formula (I′) or Formula (I) is a bivalent compound comprising an E3 ligase binding moiety and a targeting moiety (i.e., B, a binder of a target, wherein the target is selected from a protein, polypeptide, or peptide).

Provided herein are compounds of Formula (I′):

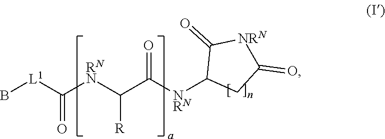

or a pharmaceutically acceptable salt or tautomer thereof, wherein:
B is hydrogen, optionally substituted alkyl, halogen, or a binder of a target, wherein the target is selected from a protein, polypeptide, peptide, carbohydrate, and small molecule;
L¹ is a bond, optionally substituted C₁₋₂₀ alkylene, optionally substituted C₂₋₂₀ alkenylene, optionally substituted C₂₋₂₀ alkynylene, optionally substituted C₁₋₂₀ heteroalkylene, optionally substituted C₂₋₂₀ heteroalkenylene, or optionally substituted C₂₋₂₀ heteroalkynylene, wherein:
   optionally one or more backbone carbon atoms of the optionally substituted alkylene, optionally substituted alkenylene, optionally substituted alkynylene, optionally substituted heteroalkylene, optionally substituted heteroalkenylene, and optionally substituted heteroalkynylene are independently replaced with —O—, —S—, —NR^{A}—, —C(═O)—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene; and optionally one or more backbone heteroatoms of the optionally substituted heteroalkylene, optionally substituted heteroalkenylene, and optionally substituted heteroalkynylene are independently replaced with —O—, —S—, —NR^{A}—, —C(═O)—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene;
R^{N} is hydrogen, optionally substituted alkyl, acyl, or a nitrogen protecting group;
R is hydrogen, optionally substituted alkyl, optionally substituted carbocyclyl, optionally substituted aryl, optionally substituted heteroaryl, or -L¹-B;
   optionally where R and R^{N} are joined together to form an optionally substituted 6-membered ring or optionally substituted 5-membered ring;
   a is selected from 0, 1, 2, 3, 4, or 5; and
   n is selected from 1, 2, and 3;
provided that only one instance of B is a binder of a target.

In some embodiments, a compound of Formula (I′) is of the formula:

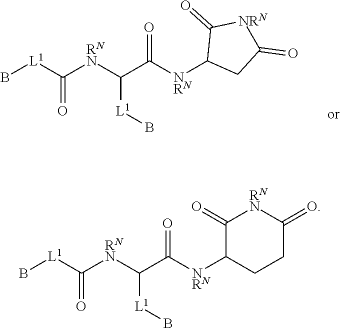

In some embodiments, a compound of Formula (I′) is of the formula:

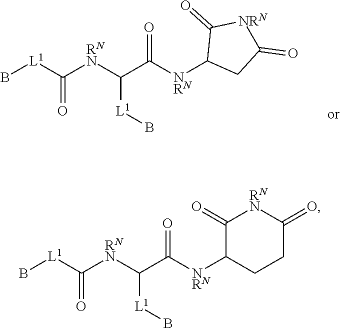

wherein

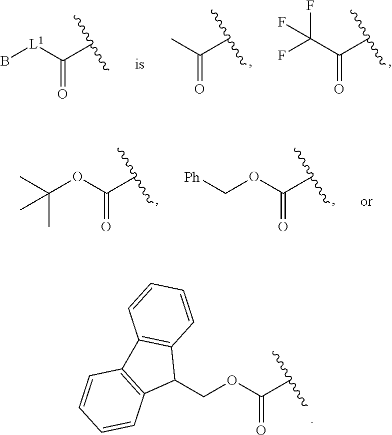

In some embodiments, a compound of Formula (I′) is of the formula:

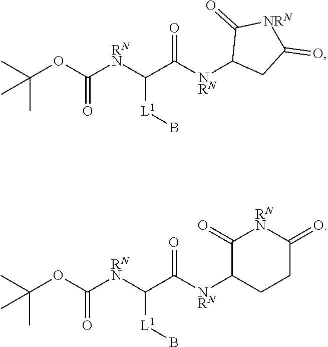

Provided herein are compounds of Formula (I′) or (I):

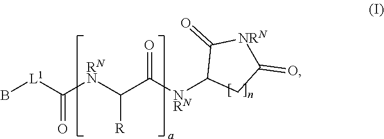

or a pharmaceutically acceptable salt or tautomer thereof, wherein:
B is hydrogen, optionally substituted alkyl, halogen, or a binder of a target, wherein the target is selected from a protein, polypeptide, peptide, carbohydrate, and small molecule;
   L¹ is a bond, optionally substituted C₁₋₂₀ alkylene, optionally substituted C₂₋₂₀ alkenylene, optionally substituted C₂₋₂₀ alkynylene, optionally substituted C₁₋₂₀ heteroalkylene, optionally substituted C₂₋₂₀ heteroalkenylene, or optionally substituted C₂₋₂₀ heteroalkynylene, wherein:
   optionally one or more backbone carbon atoms of the optionally substituted alkylene, optionally substituted alkenylene, optionally substituted alkynylene, optionally substituted heteroalkylene, optionally substituted heteroalkenylene, and optionally substituted heteroalkynylene are independently replaced with —O—, —S—, —NR^{A}—, —C(═O)—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene; and
   optionally one or more backbone heteroatoms of the optionally substituted heteroalkylene, optionally substituted heteroalkenylene, and optionally substituted heteroalkynylene are independently replaced with —O—, —S—, —NR^{A}, —C(═O)—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene;
R^{N} is hydrogen, optionally substituted alkyl, acyl, or a nitrogen protecting group;
R is hydrogen or optionally substituted alkyl;
   optionally where R and R^{N} are joined together to form a 5-membered ring;
a is selected from 0, 1, 2, 3, 4, and 5; and
n is selected from 1, 2, and 3.

Provided herein are compounds of Formula (I′) or (I):

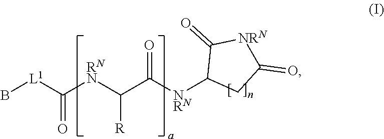

or a pharmaceutically acceptable salt or tautomer thereof, wherein:
B is hydrogen, optionally substituted alkyl, halogen, or a binder of a target, wherein the target is selected from a protein, polypeptide, peptide, carbohydrate, and small molecule;
   L¹ is a bond, optionally substituted C₁₋₂₀ alkylene, optionally substituted C₂₋₂₀ alkenylene, optionally substituted C₂₋₂₀ alkynylene, optionally substituted C₁₋₂₀ heteroalkylene, optionally substituted C₂₋₂₀ heteroalkenylene, or optionally substituted C₂₋₂₀ heteroalkynylene, wherein:
   optionally one or more backbone carbon atoms of the optionally substituted alkylene, optionally substituted alkenylene, optionally substituted alkynylene, optionally substituted heteroalkylene, optionally substituted heteroalkenylene, and optionally substituted heteroalkynylene are independently replaced with —O—, —S—, —NR^{A}—, —C(═O)—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene; and
   optionally one or more backbone heteroatoms of the optionally substituted heteroalkylene, optionally substituted heteroalkenylene, and optionally substituted heteroalkynylene are independently replaced with —O—, —S—, —NR^{A}, —C(═O)—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene;
R^{N} is hydrogen, optionally substituted alkyl, acyl, or a nitrogen protecting group;
R is hydrogen or optionally substituted alkyl;
   optionally where R and R^{N} are joined together to form a 5-membered ring;
a is selected from 0, 1, and 2; and
n is selected from 1, 2, and 3.

Provided herein are compounds of Formula (I′) or (I):

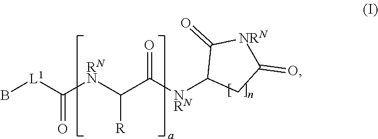

or a pharmaceutically acceptable salt or tautomer thereof, wherein:
B is hydrogen, optionally substituted alkyl, halogen, or a binder of a target, wherein the target is selected from a protein, polypeptide, peptide, carbohydrate, and small molecule;
   L¹ is a bond, optionally substituted C₁₋₂₀ alkylene, optionally substituted C₂₋₂₀ alkenylene, optionally substituted C₂₋₂₀ alkynylene, optionally substituted C₁₋₂₀ heteroalkylene, optionally substituted C₂₋₂₀ heteroalkenylene, or optionally substituted C₂₋₂₀ heteroalkynylene, wherein:
   optionally one or more backbone carbon atoms of the optionally substituted alkylene, optionally substituted alkenylene, optionally substituted alkynylene, optionally substituted heteroalkylene, optionally substituted heteroalkenylene, and optionally substituted heteroalkynylene are independently replaced with —O—, —S—, —NR^{A}—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene; and
   optionally one or more backbone heteroatoms of the optionally substituted heteroalkylene, optionally substituted heteroalkenylene, and optionally substituted heteroalkynylene are independently replaced with —O—, —S—, —NR^{A}—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene;
R^{N} is hydrogen, optionally substituted alkyl, acyl, or a nitrogen protecting group;
R is hydrogen or optionally substituted alkyl;
   optionally where R and R^{N} are joined together to form a 5-membered ring;
a is selected from 0, 1, and 2; and
n is selected from 1, 2, and 3.

In some embodiments, a compound of Formula (I′) or (I) is of the formula:

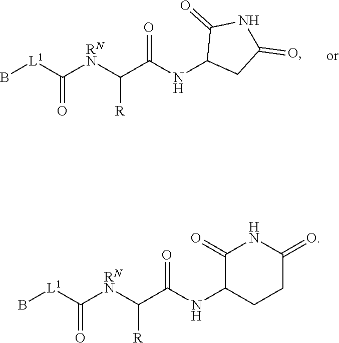

In some embodiments, a compound of Formula (I′) or (I) is of the formula:

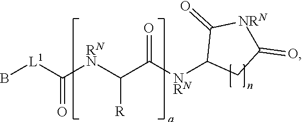

or a pharmaceutically acceptable salt or tautomer thereof, wherein L¹ is optionally substituted C₁₋₂₀ alkylene or optionally substituted C₁₋₂₀ heteroalkylene; and B is hydrogen. In some embodiments, -L¹-B is optionally substituted alkoxy or optionally substituted alkyl. In some embodiments, -L¹-B is selected from the group consisting of —O^{t}Bu, —OCH₂Ph, —OCH₂— (fluorenyl), —CF₃, and —CH₃.

In some embodiments, a compound of Formula (I′) or (I) is of the formula:

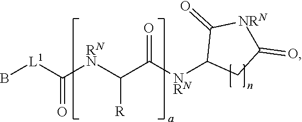

or a pharmaceutically acceptable salt or tautomer thereof, wherein B is binder of a target wherein the target is selected from a protein, polypeptide, peptide, carbohydrate, and small molecule. In some embodiments, B is a binder of a target selected from the group consisting of a bromodomain, a bromodomain-containing protein, a histone methyltransferase, a kinase, a cytosolic signaling protein, a nuclear protein, a histone deacetylase, a lysine methyltransferase, a protein regulating angiogenesis, a protein regulating immune response, an aryl hydrocarbon receptor, a hormone receptor, and a transcription factor. In certain embodiments, L¹ comprises more than four non-hydrogen atoms (i.e., the shortest path from B to the carbonyl carbon is four atoms in length).

In some embodiments R is hydrogen, optionally substituted alkyl, optionally substituted carbocyclyl, optionally substituted aryl, optionally substituted heteroaryl, or -L¹-B; optionally where R and R^{N} are joined together to form an optionally substituted 6-membered ring or optionally substituted 5-membered ring.

In certain embodiments, R is hydrogen or optionally substituted alkyl. In some embodiments, R is hydrogen. In certain embodiments, R is optionally substituted alkyl. In some embodiments, R is substituted alkyl. In some embodiments, R is hydrogen or C₁₋₆ alkyl substituted with —OR^{O}, —O⁻, —SR^{S}, —NR^{N}, —NH₃⁺, —C(═O)N(R^{A})₂, —C(═O)OR^{O}, —C(═O)O⁻, —N(R^{A})C(═NR^{A})N(R^{A})₂, —N(R^{A})C(═N⁺(R^{A})₂) N(R^{A})₂, optionally substituted aryl, optionally substituted heteroaryl, optionally substituted carbocyclyl, or optionally substituted heterocyclyl, wherein: R^{O} is hydrogen, optionally substituted alkyl, or an oxygen protecting group; R^{S} is hydrogen, optionally substituted alkyl, or a sulfur protection group; and R^{A} is hydrogen, optionally substituted alkyl, or a nitrogen protecting group. In some embodiments, R is hydrogen or C₁₋₆ alkyl substituted with —OR^{O}, —O⁻, —SR^{S}, —N(R^{N})₂, —NH₃⁺, —C(═O)N(R^{A})₂, —C(═O)OR^{O}, —C(═O)O⁻, —N(R^{A})C(═NR^{A})N(R^{A})₂, —N(R^{A})C(═N⁺(R^{A})₂) N(R^{A})₂, optionally substituted aryl, optionally substituted heteroaryl, optionally substituted carbocyclyl, or optionally substituted heterocyclyl, wherein: R^{O} is hydrogen, optionally substituted alkyl, or an oxygen protecting group; R^{S} is hydrogen, optionally substituted alkyl, or a sulfur protection group; and R^{A} is hydrogen, optionally substituted alkyl, or a nitrogen protecting group. In some embodiments, R is hydrogen or C₁₋₆ alkyl substituted with —OR^{O}, —O⁻, —SR^{S}, —N(R^{A})₂, —NH₃⁺, —C(═O)N(R^{A})₂, —C(═O)OR^{O}, —C(═O)O⁻, —N(R^{A})C(═NR^{A})N(R^{A})₂, —N(R^{A})C(═N⁺(R^{A})₂) N(R^{A})₂, optionally substituted aryl, optionally substituted heteroaryl, optionally substituted carbocyclyl, or optionally substituted heterocyclyl, wherein: R^{O} is hydrogen, optionally substituted alkyl, or an oxygen protecting group; R^{S} is hydrogen, optionally substituted alkyl, or a sulfur protection group; and R^{A} is hydrogen, optionally substituted alkyl, or a nitrogen protecting group. In some embodiments, R is C₁₋₆ alkyl substituted with —OH, —O⁻, —SH, —NH₂, —NMe₂, —NH₃⁺, —C(═O)NH₂, —C(═O)NMe₂, —C(═O)OH, —C(═O)O⁻, —NHC(═NH)NH₂, —NHC(—NH₂+)NH₂, phenyl, phenyl substituted with —OH or —NH₂, indolyl, or imidazolyl.

In certain embodiments, R is an amino acid side chain or derivative thereof (e.g., an amino acid analog). In some embodiments, R is selected from the group consisting of:

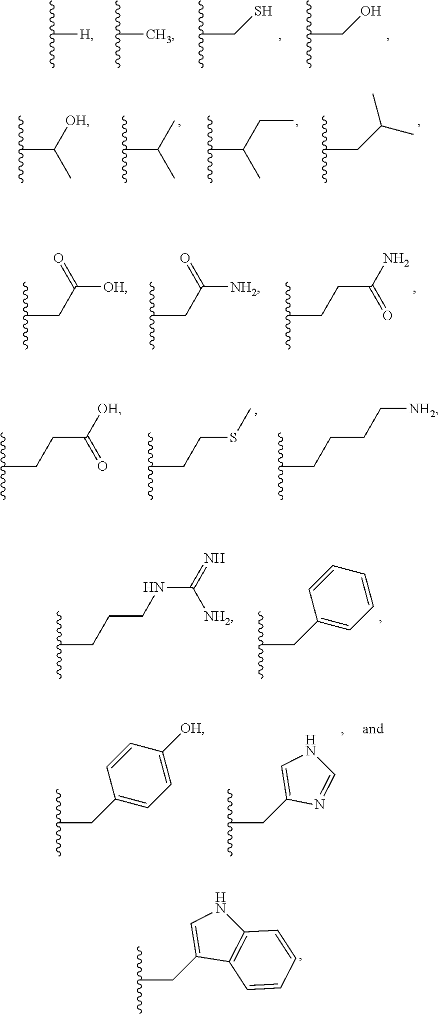

or a pharmaceutically acceptable salt or tautomer thereof. In certain embodiments. R is selected from the group consisting of:

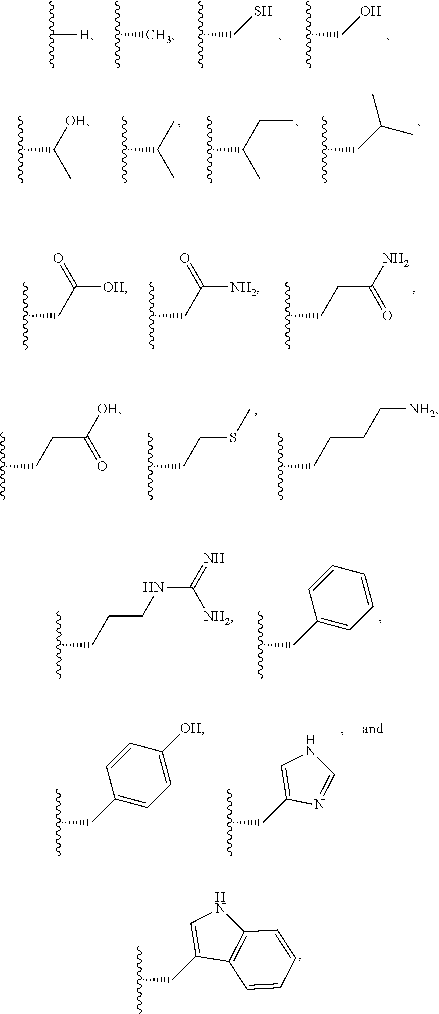

or a pharmaceutically acceptable salt or tautomer thereof. In some embodiments, R is an amino acid analog. In some embodiments, R is selected from the group consisting of:

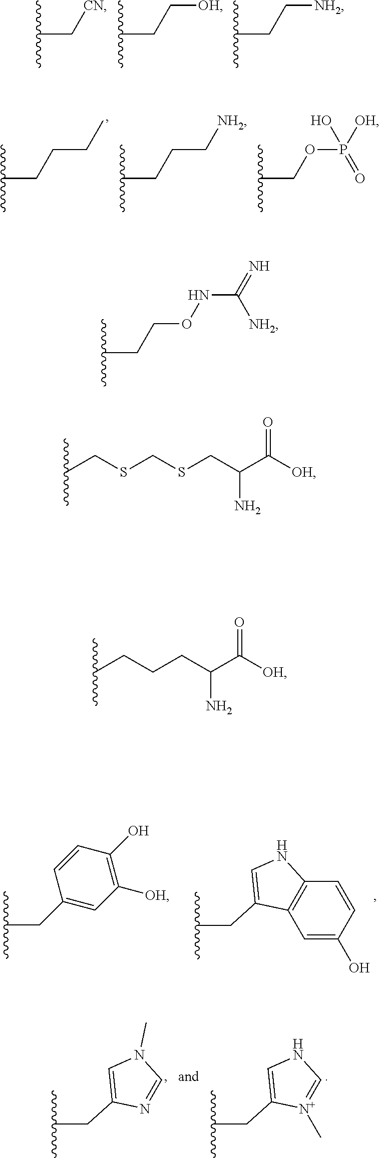

In some embodiments R is hydrogen, optionally substituted alkyl, optionally substituted carbocyclyl, optionally substituted aryl, optionally substituted heteroaryl, or -L¹-B. In some embodiments, R is -L¹-B. In some embodiments, R is C₃₋₈ carbocyclyl, C₁₋₆ alkyl, optionally substituted phenylmethyl, optionally substituted naphthalenylmethyl, or C₃₋₈ carbocyclylmethyl. In some embodiments, R is cyclopropyl, cyclobutyl, cyclopentyl, cyclohexyl, ethyl, propyl, butyl, tert-butyl, cyclopropylmethyl, cyclobutylmethyl, cyclopentylmethyl, cyclohexylmethyl, naphthalenylmethyl, phenylmethyl, or substituted phenylmethyl. In some embodiments, R is phenylmethyl substituted with methyl, halogen, acyl, —NO₂, or —NH₂.

In some embodiments R is selected from the group consist of:

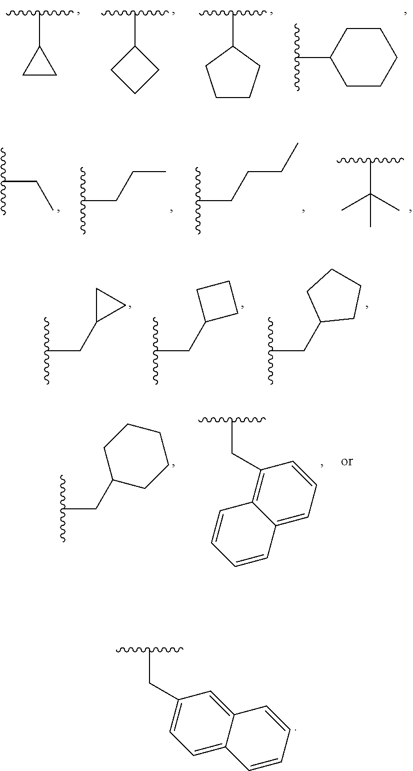

In some embodiments, R and R^{N} are joined together to form a ring. In certain embodiments, R and the R^{N} on the nitrogen adjacent to the carbon to which R is attached are joined to form a ring

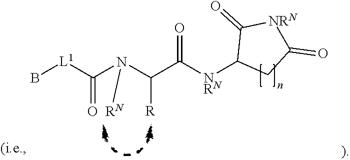

In some embodiments, R and R^{N} are joined together to form a 5-membered ring. In some embodiments, R and R^{N} are joined together to form a 5-membered ring, comprising two methylene units between R and R^{N}. In some embodiments, R and R^{N} are joined together to form a 6-membered ring. In some embodiments, R and R^{N} are joined together to form a 6-membered ring, comprising three methylene units between R and R^{N}. In some embodiments, R and R^{N} are joined together to form an optionally substituted 5-membered ring. In some embodiments, R and R^{N} are joined together to form an optionally substituted 6-membered ring. In some embodiments, R and R^{N} are joined together to form a ring:

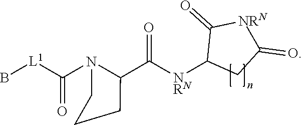

In some embodiments, R and R^{N} are joined together to form a ring:

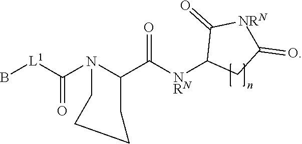

In some embodiments, R and R^{N} are joined together to form a ring:

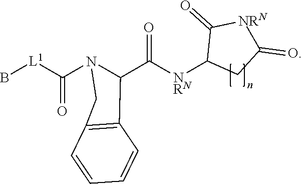

In some embodiments, R and R^{N} are joined together to form a ring:

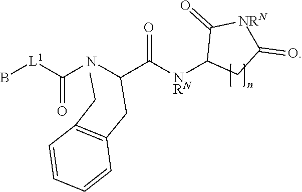

In some embodiments, R and R^{N} are joined to form a ring:

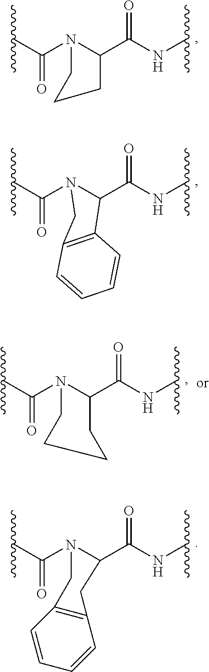 [SMILES: *NC(=O)C1CCCN1C(*)=O ; *NC(=O)C1c2ccccc2CN1C(*)=O ; *NC(=O)C1CCCCN1C(*)=O ; *NC(=O)C1Cc2ccccc2CN1C(*)=O]

In some embodiments, R^{N} is hydrogen, optionally substituted alkyl, acyl, or a nitrogen protecting group. In certain embodiments, each instance of R^{N} is hydrogen. In some embodiments, two instances of R^{N} are hydrogen. In certain embodiments, one instance of R^{N} is acyl or optionally substituted alkyl. In certain embodiments, each instance of R^{N} is acyl or optionally substituted alkyl. In certain embodiments, each instance of R^{N} is methyl. In some embodiments, two instances of R^{N} are methyl. In certain embodiments, one instance of R^{N} is a nitrogen protecting group. In certain embodiments, each instance of R^{N} is a nitrogen protecting group. In some embodiments, one instance of R^{N} is —C(═O)O^{t}Bu, —C(═O)OCH₂Ph, —C(═O)OCH₂-(fluorenyl), —C(═O)CF₃, and —C(═O)CH₃. In some embodiments, each instance of R^{N} is —C(═O)O^{t}Bu, —C(═O)OCH₂Ph, —C(═O)OCH₂-(fluorenyl), —C(═O)CF₃, and —C(═O)CH₃.

In some embodiments, n is 2. In certain embodiments, n is 1. In some embodiments, n is 3.

In some embodiments, a is selected from 0, 1, 2, 3, 4, and 5. In some embodiments, a is selected from 0, 1, and 2. In some embodiments, preferably, a is 1. In some embodiments, a is 0. In some embodiments, a is 2. In some embodiments, a is 3. In some embodiments, a is 4. In certain embodiments, a is 5.

In some embodiments,

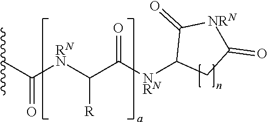

is of the formula:

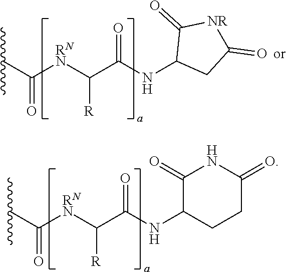

In some embodiments,

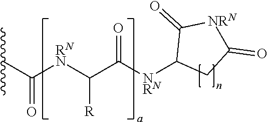

is of the formula:

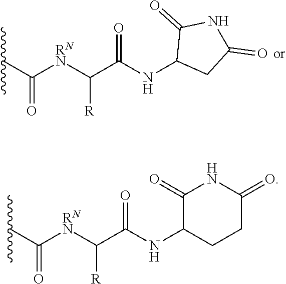

In some embodiments,

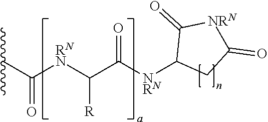

is of the formula:

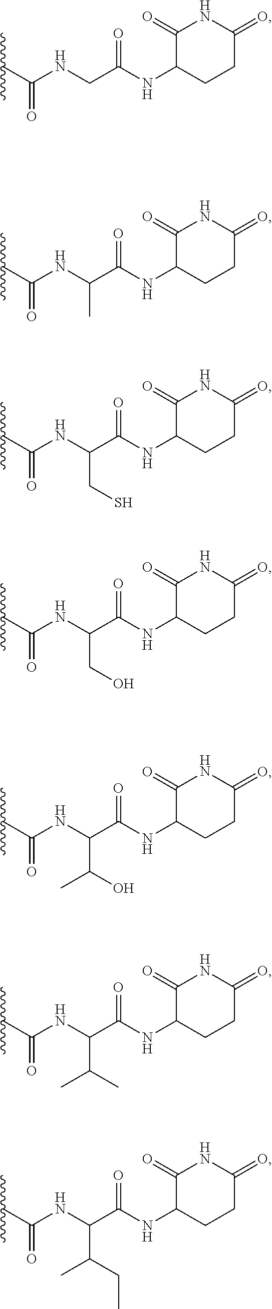

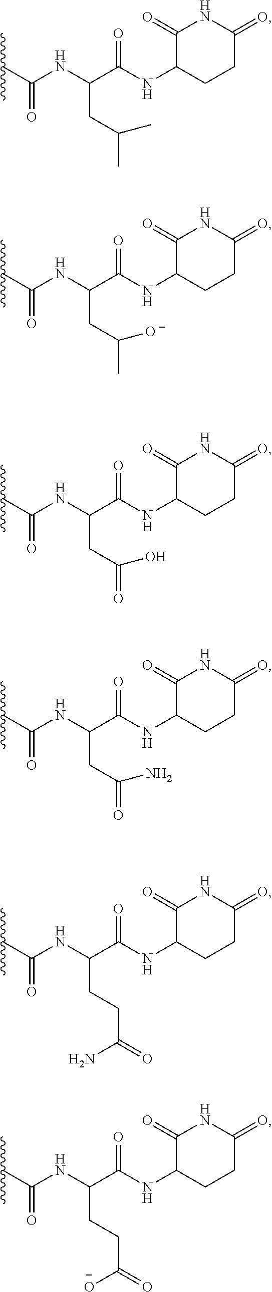 [SMILES: *C(=O)NC(CC(C)C)C(=O)NC1CCC(=O)NC1=O ; *C(=O)NC(CC(C)[O-])C(=O)NC1CCC(=O)NC1=O ; *C(=O)NC(CC(=O)O)C(=O)NC1CCC(=O)NC1=O ; *C(=O)NC(CC(N)=O)C(=O)NC1CCC(=O)NC1=O ; *C(=O)NC(CCC(N)=O)C(=O)NC1CCC(=O)NC1=O ; *C(=O)NC(CCC(=O)[O-])C(=O)NC1CCC(=O)NC1=O]

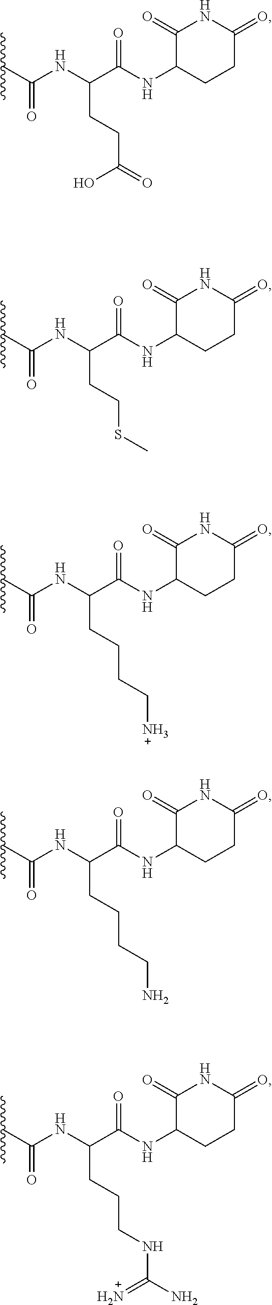 [SMILES: *C(=O)NC(CCC(=O)O)C(=O)NC1CCC(=O)NC1=O ; *C(=O)NC(CCSC)C(=O)NC1CCC(=O)NC1=O ; *C(=O)NC(CCCC[NH3+])C(=O)NC1CCC(=O)NC1=O ; *C(=O)NC(CCCCN)C(=O)NC1CCC(=O)NC1=O ; *C(=O)NC(CCCNC(N)=[NH2+])C(=O)NC1CCC(=O)NC1=O]

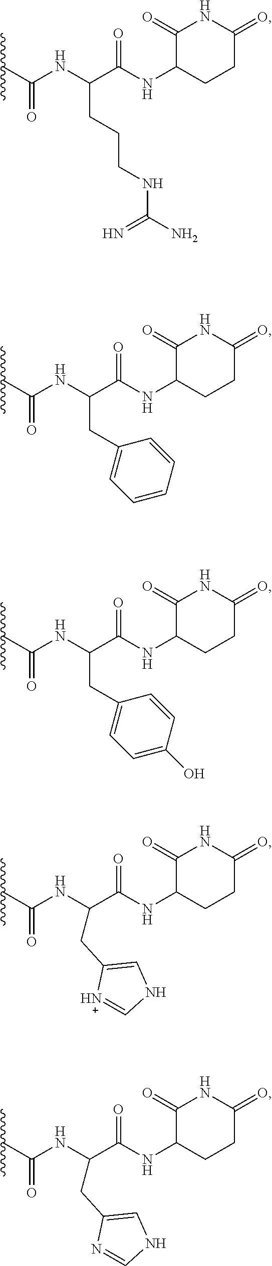 [SMILES: *C(=O)NC(CCCNC(=N)N)C(=O)NC1CCC(=O)NC1=O ; *C(=O)NC(Cc1ccccc1)C(=O)NC1CCC(=O)NC1=O ; *C(=O)NC(Cc1ccc(O)cc1)C(=O)NC1CCC(=O)NC1=O ; *C(=O)NC(Cc1c[nH]c[nH+]1)C(=O)NC1CCC(=O)NC1=O ; *C(=O)NC(Cc1c[nH]cn1)C(=O)NC1CCC(=O)NC1=O]

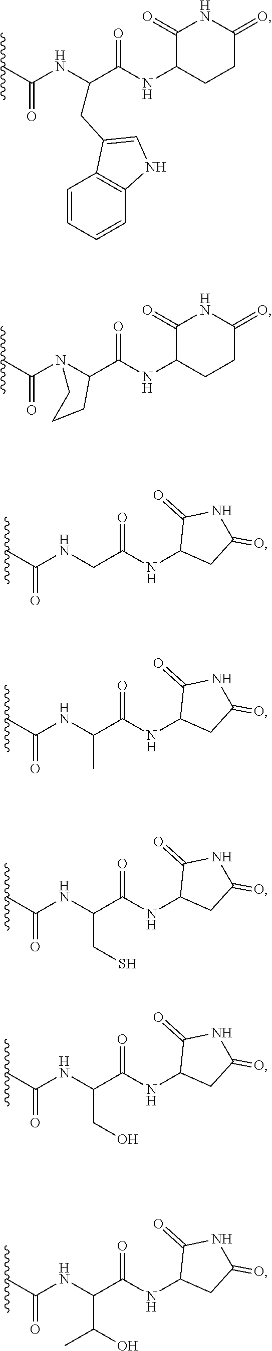

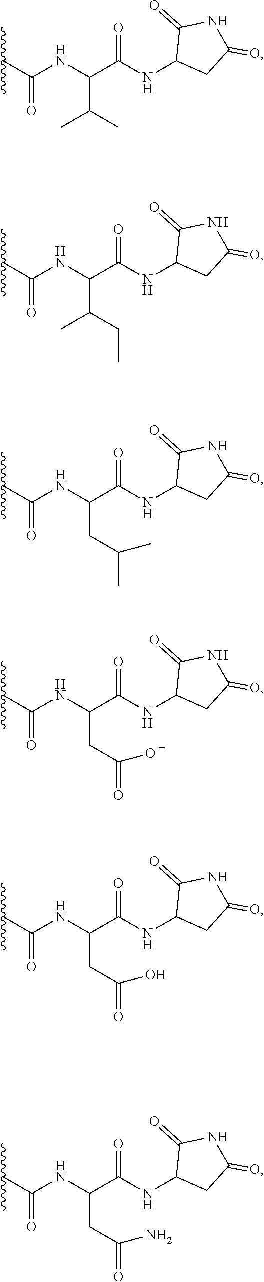 [SMILES: *C(=O)NC(C(=O)NC1CC(=O)NC1=O)C(C)C ; *C(=O)NC(C(=O)NC1CC(=O)NC1=O)C(C)CC ; *C(=O)NC(CC(C)C)C(=O)NC1CC(=O)NC1=O ; *C(=O)NC(CC(=O)[O-])C(=O)NC1CC(=O)NC1=O ; *C(=O)NC(CC(=O)O)C(=O)NC1CC(=O)NC1=O ; *C(=O)NC(CC(N)=O)C(=O)NC1CC(=O)NC1=O]

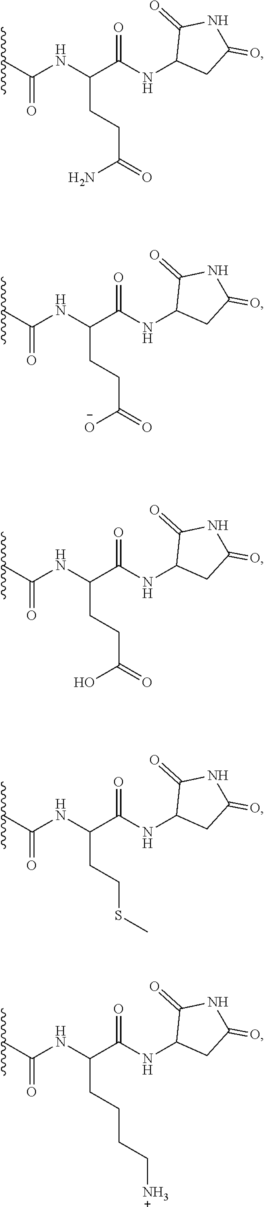 [SMILES: *C(=O)NC(CCC(N)=O)C(=O)NC1CC(=O)NC1=O ; *C(=O)NC(CCC(=O)[O-])C(=O)NC1CC(=O)NC1=O ; *C(=O)NC(CCC(=O)O)C(=O)NC1CC(=O)NC1=O ; *C(=O)NC(CCSC)C(=O)NC1CC(=O)NC1=O ; *C(=O)NC(CCCC[NH3+])C(=O)NC1CC(=O)NC1=O]

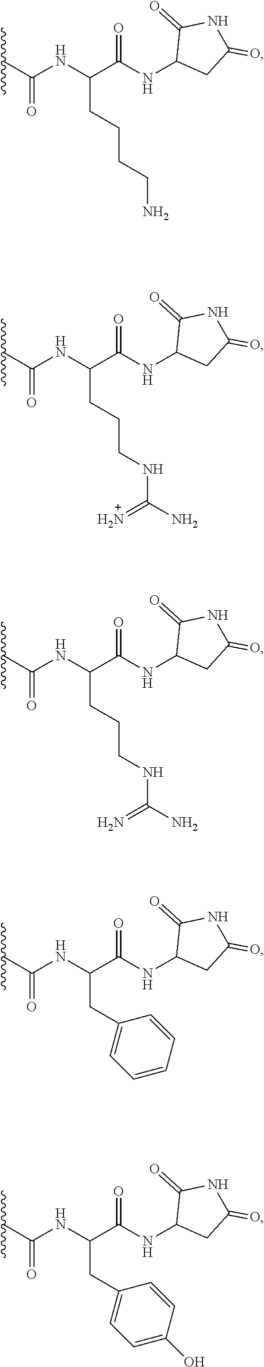 [SMILES: *C(=O)NC(CCCCN)C(=O)NC1CC(=O)NC1=O ; *C(=O)NC(CCCNC(N)=[NH2+])C(=O)NC1CC(=O)NC1=O ; *C(=O)NC(CCCNC(=N)N)C(=O)NC1CC(=O)NC1=O ; *C(=O)NC(Cc1ccccc1)C(=O)NC1CC(=O)NC1=O ; *C(=O)NC(Cc1ccc(O)cc1)C(=O)NC1CC(=O)NC1=O]

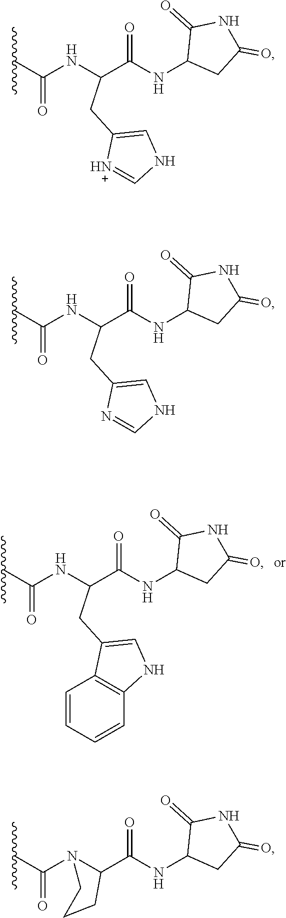 [SMILES: *C(=O)NC(Cc1c[nH]c[nH+]1)C(=O)NC1CC(=O)NC1=O ; *C(=O)NC(Cc1c[nH]cn1)C(=O)NC1CC(=O)NC1=O ; *C(=O)NC(Cc1c[nH]c2ccccc12)C(=O)NC1CC(=O)NC1=O ; "or" ; *C(=O)N1CCCC1C(=O)NC1CC(=O)NC1=O]

or a pharmaceutically acceptable salt or tautomer thereof. In some embodiments,

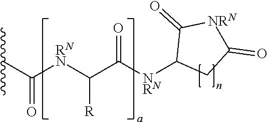

is of the formula:

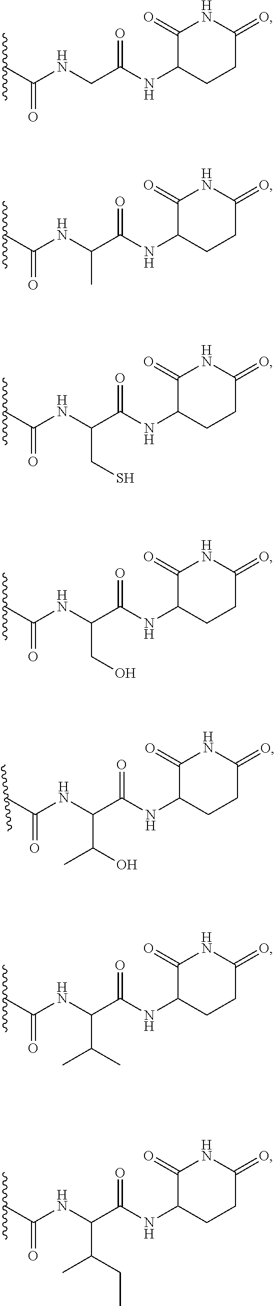

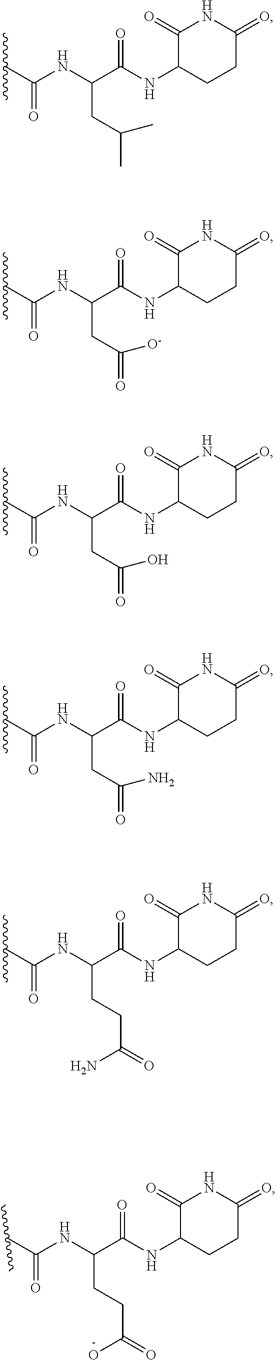 [SMILES: *C(=O)NC(CC(C)C)C(=O)NC1CCC(=O)NC1=O ; *C(=O)NC(CC(=O)[O-])C(=O)NC1CCC(=O)NC1=O ; *C(=O)NC(CC(=O)O)C(=O)NC1CCC(=O)NC1=O ; *C(=O)NC(CC(N)=O)C(=O)NC1CCC(=O)NC1=O ; *C(=O)NC(CCC(N)=O)C(=O)NC1CCC(=O)NC1=O ; *C(=O)NC(CCC(=O)O)C(=O)NC1CCC(=O)NC1=O]

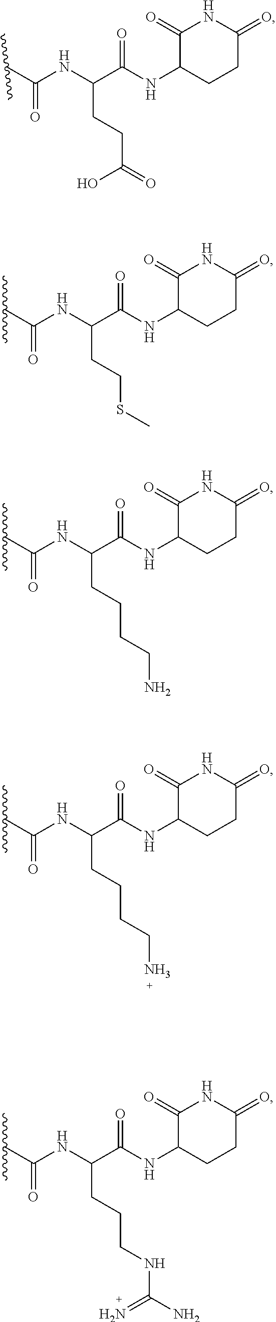 [SMILES: *C(=O)NC(CCC(=O)O)C(=O)NC1CCC(=O)NC1=O ; *C(=O)NC(CCSC)C(=O)NC1CCC(=O)NC1=O ; *C(=O)NC(CCCCN)C(=O)NC1CCC(=O)NC1=O ; *C(=O)NC(CCCCN)C(=O)NC1CCC(=O)NC1=O ; *C(=O)NC(CCCNC(=N)N)C(=O)NC1CCC(=O)NC1=O]

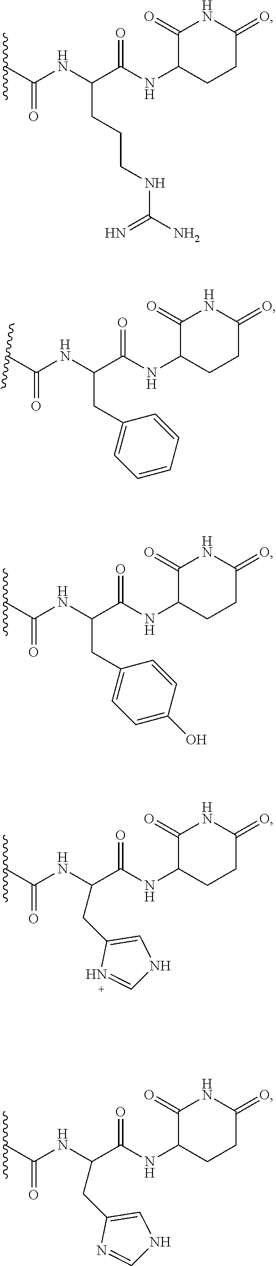 [SMILES: *C(=O)NC(CCCNC(=N)N)C(=O)NC1CCC(=O)NC1=O ; *C(=O)NC(Cc1ccccc1)C(=O)NC1CCC(=O)NC1=O ; *C(=O)NC(Cc1ccc(O)cc1)C(=O)NC1CCC(=O)NC1=O ; *C(=O)NC(Cc1c[nH]cn1)C(=O)NC1CCC(=O)NC1=O ; *C(=O)NC(Cc1c[nH]cn1)C(=O)NC1CCC(=O)NC1=O]

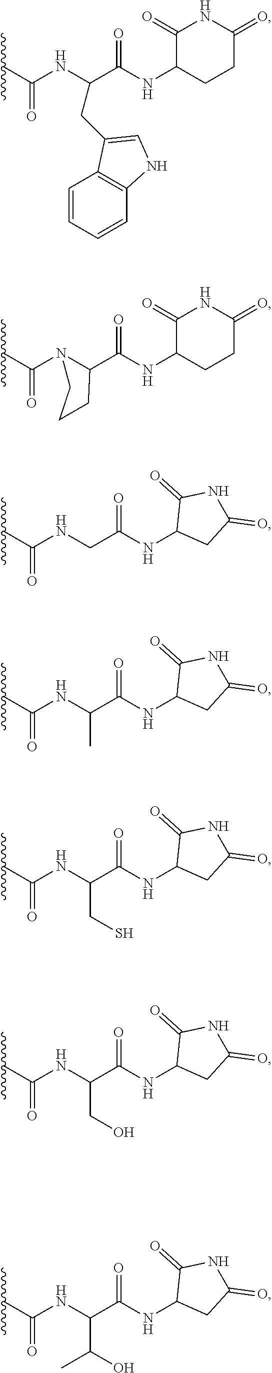

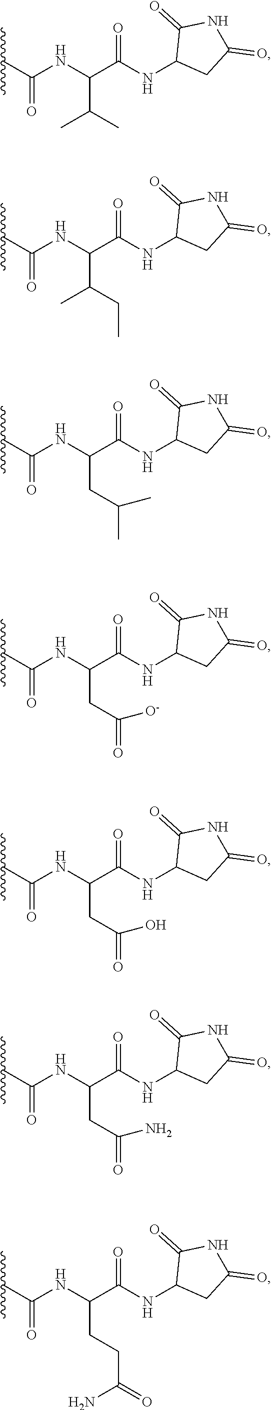

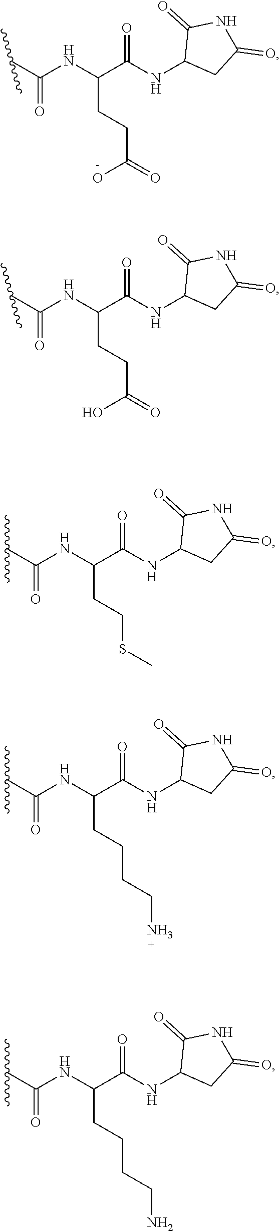 [SMILES: *C(=O)NC(CCC(=O)O)C(=O)NC1CC(=O)NC1=O ; *C(=O)NC(CCC(=O)O)C(=O)NC1CC(=O)NC1=O ; *C(=O)NC(CCSC)C(=O)NC1CC(=O)NC1=O ; *C(=O)NC(CCCCN)C(=O)NC1CC(=O)NC1=O ; *C(=O)NC(CCCCN)C(=O)NC1CC(=O)NC1=O]

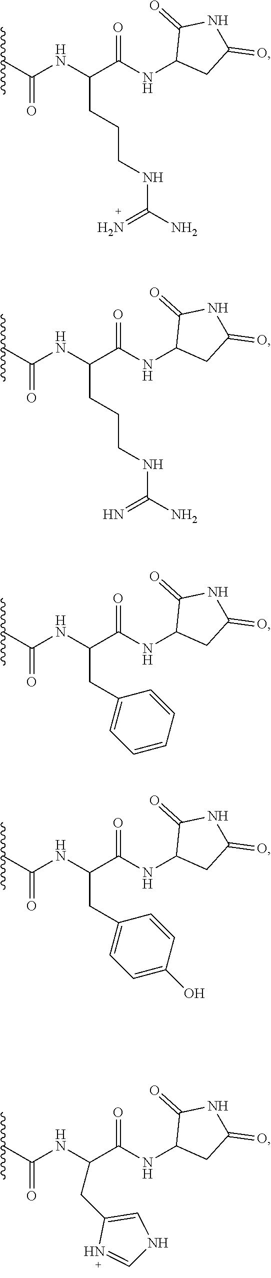 [SMILES: *C(=O)NC(CCCNC(=N)N)C(=O)NC1CC(=O)NC1=O ; *C(=O)NC(CCCNC(=N)N)C(=O)NC1CC(=O)NC1=O ; *C(=O)NC(Cc1ccccc1)C(=O)NC1CC(=O)NC1=O ; *C(=O)NC(Cc1ccc(O)cc1)C(=O)NC1CC(=O)NC1=O ; *C(=O)NC(Cc1c[nH]cn1)C(=O)NC1CC(=O)NC1=O]

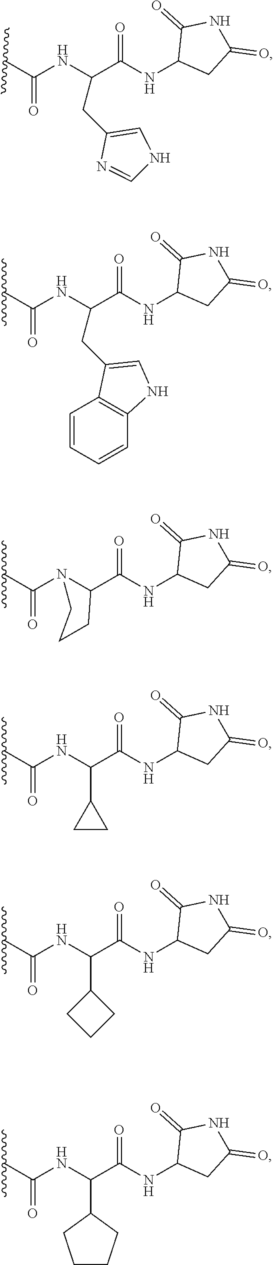 [SMILES: *C(=O)NC(Cc1c[nH]cn1)C(=O)NC1CC(=O)NC1=O ; *C(=O)NC(Cc1c[nH]c2ccccc12)C(=O)NC1CC(=O)NC1=O ; *C(=O)N1CCCC1C(=O)NC1CC(=O)NC1=O ; *C(=O)NC(C(=O)NC1CC(=O)NC1=O)C1CC1 ; *C(=O)NC(C(=O)NC1CC(=O)NC1=O)C1CCC1 ; *C(=O)NC(C(=O)NC1CC(=O)NC1=O)C1CCCC1]

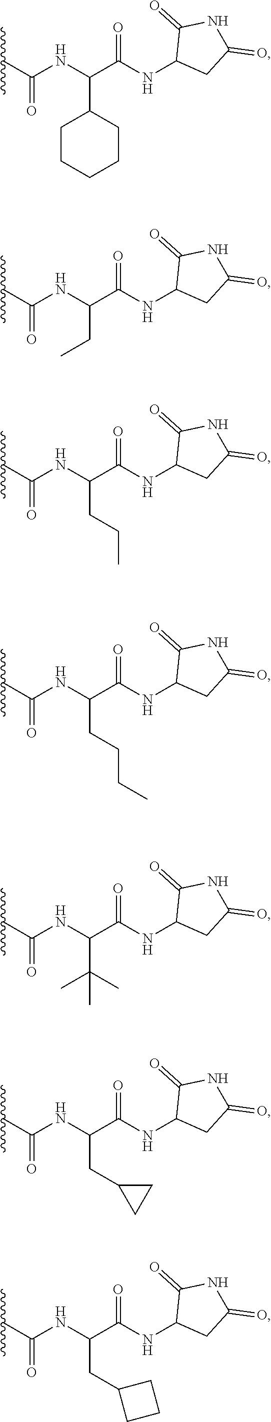

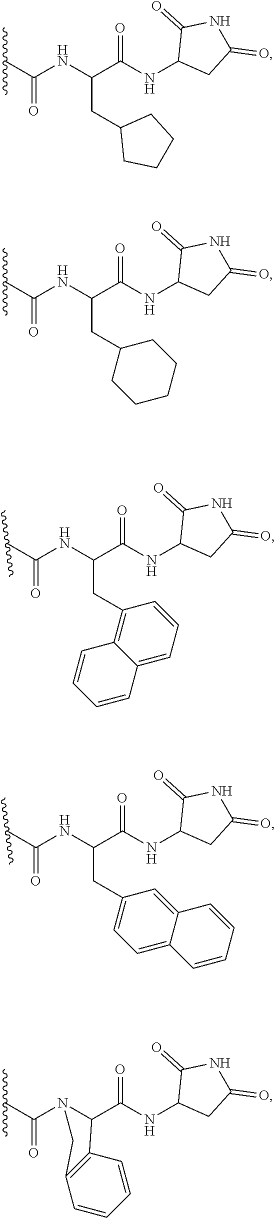 [SMILES: *C(=O)NC(CC1CCCC1)C(=O)NC1CC(=O)NC1=O ; *C(=O)NC(CC1CCCCC1)C(=O)NC1CC(=O)NC1=O ; *C(=O)NC(Cc1cccc2ccccc12)C(=O)NC1CC(=O)NC1=O ; *C(=O)NC(Cc1ccc2ccccc2c1)C(=O)NC1CC(=O)NC1=O ; *C(=O)N1Cc2ccccc2C1C(=O)NC1CC(=O)NC1=O]

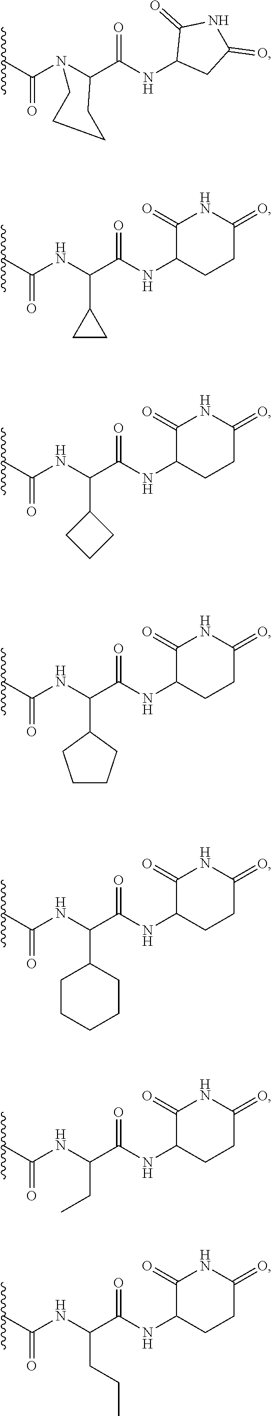

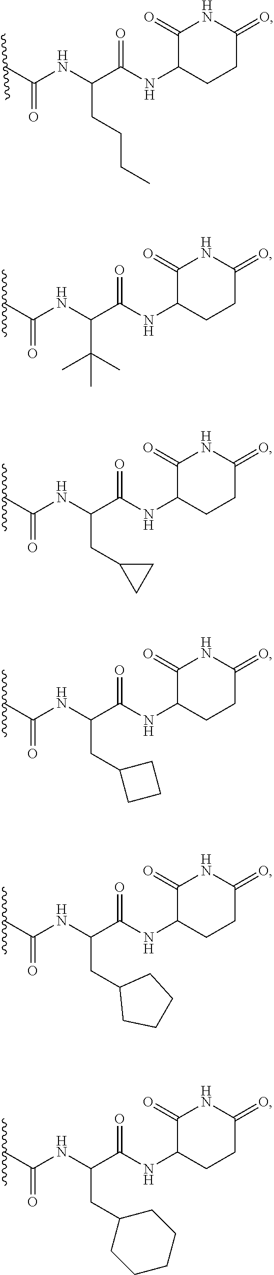 [SMILES: *C(=O)NC(CCCC)C(=O)NC1CCC(=O)NC1=O ; *C(=O)NC(C(=O)NC1CCC(=O)NC1=O)C(C)(C)C ; *C(=O)NC(CC1CC1)C(=O)NC1CCC(=O)NC1=O ; *C(=O)NC(CC1CCC1)C(=O)NC1CCC(=O)NC1=O ; *C(=O)NC(CC1CCCC1)C(=O)NC1CCC(=O)NC1=O ; *C(=O)NC(CC1CCCCC1)C(=O)NC1CCC(=O)NC1=O]

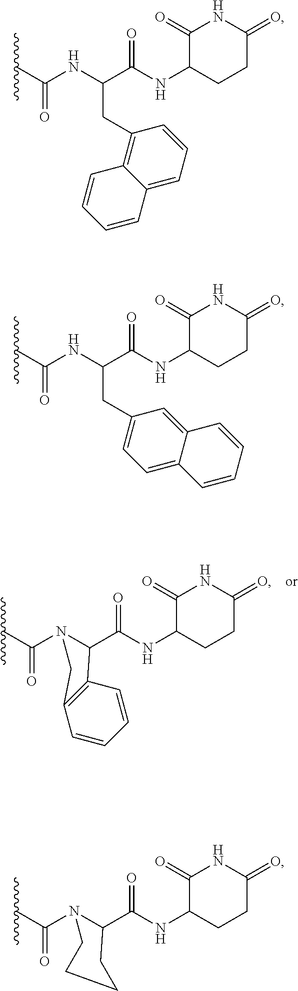 [SMILES: *C(=O)NC(Cc1cccc2ccccc12)C(=O)NC1CCC(=O)NC1=O ; *C(=O)NC(Cc1ccc2ccccc2c1)C(=O)NC1CCC(=O)NC1=O ; *C(=O)N1Cc2ccccc2C1C(=O)NC1CCC(=O)NC1=O ; *C(=O)N1CCCCC1C(=O)NC1CCC(=O)NC1=O]

or a pharmaceutically acceptable salt or tautomer thereof. In some embodiments,

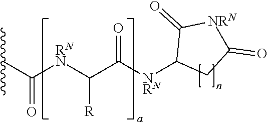

is of the formula:

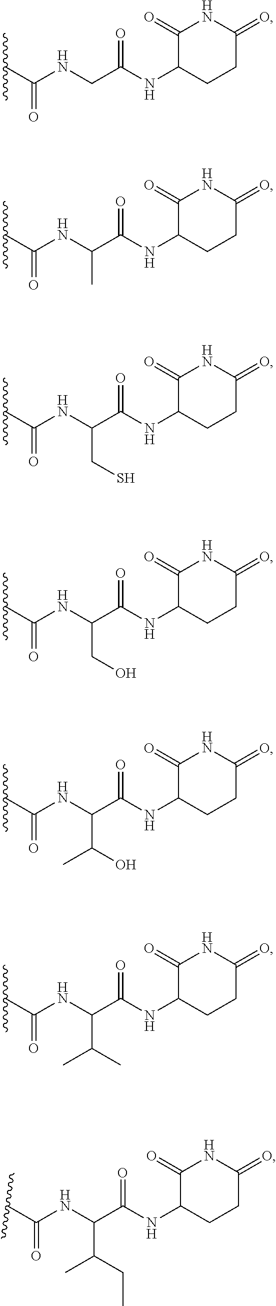

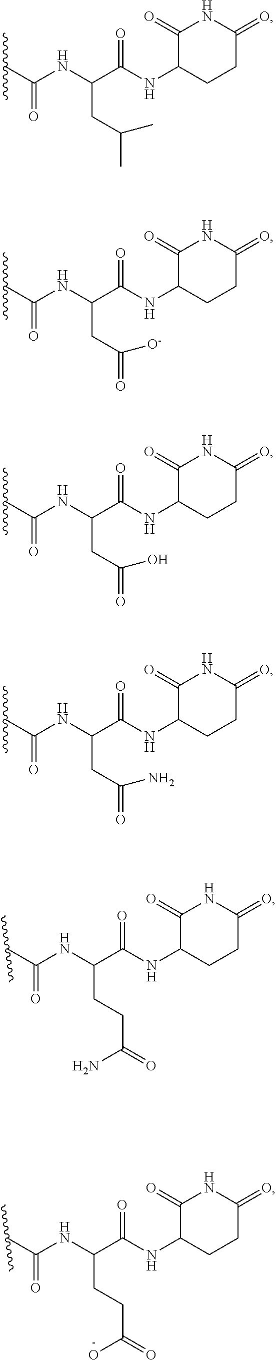 [SMILES: *C(=O)NC(CC(C)C)C(=O)NC1CCC(=O)NC1=O ; *C(=O)NC(CC(=O)[O-])C(=O)NC1CCC(=O)NC1=O ; *C(=O)NC(CC(=O)O)C(=O)NC1CCC(=O)NC1=O ; *C(=O)NC(CC(N)=O)C(=O)NC1CCC(=O)NC1=O ; *C(=O)NC(CCC(N)=O)C(=O)NC1CCC(=O)NC1=O ; *C(=O)NC(CCC(=O)O)C(=O)NC1CCC(=O)NC1=O]

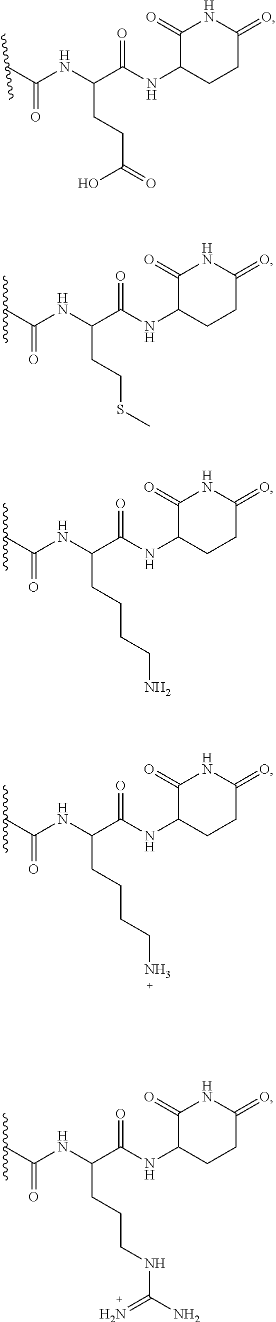 [SMILES: *C(=O)NC(CCC(=O)O)C(=O)NC1CCC(=O)NC1=O ; *C(=O)NC(CCSC)C(=O)NC1CCC(=O)NC1=O ; *C(=O)NC(CCCCN)C(=O)NC1CCC(=O)NC1=O ; *C(=O)NC(CCCCN)C(=O)NC1CCC(=O)NC1=O ; *C(=O)NC(CCCNC(=N)N)C(=O)NC1CCC(=O)NC1=O]

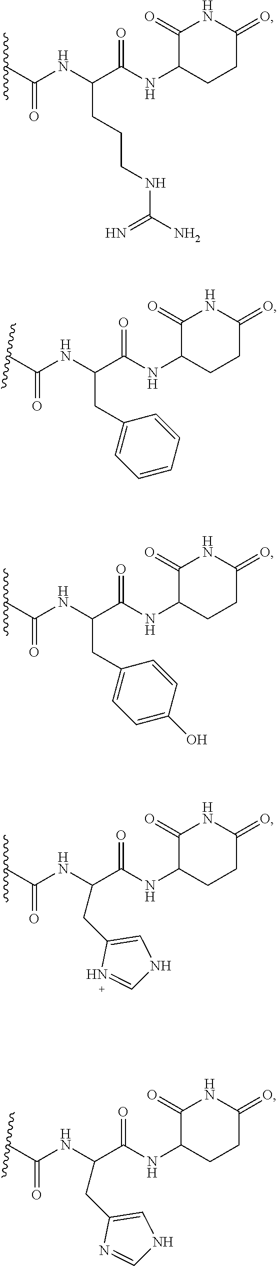 [SMILES: *C(=O)NC(CCCNC(=N)N)C(=O)NC1CCC(=O)NC1=O ; *C(=O)NC(Cc1ccccc1)C(=O)NC1CCC(=O)NC1=O ; *C(=O)NC(Cc1ccc(O)cc1)C(=O)NC1CCC(=O)NC1=O ; *C(=O)NC(Cc1c[nH]cn1)C(=O)NC1CCC(=O)NC1=O ; *C(=O)NC(Cc1c[nH]cn1)C(=O)NC1CCC(=O)NC1=O]

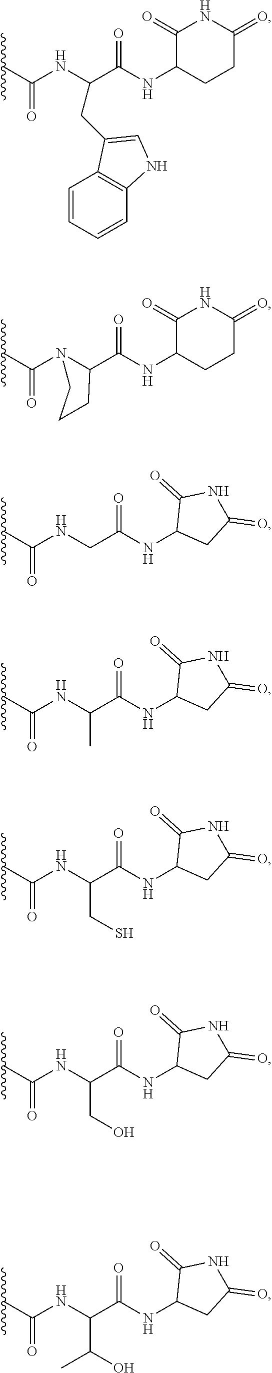

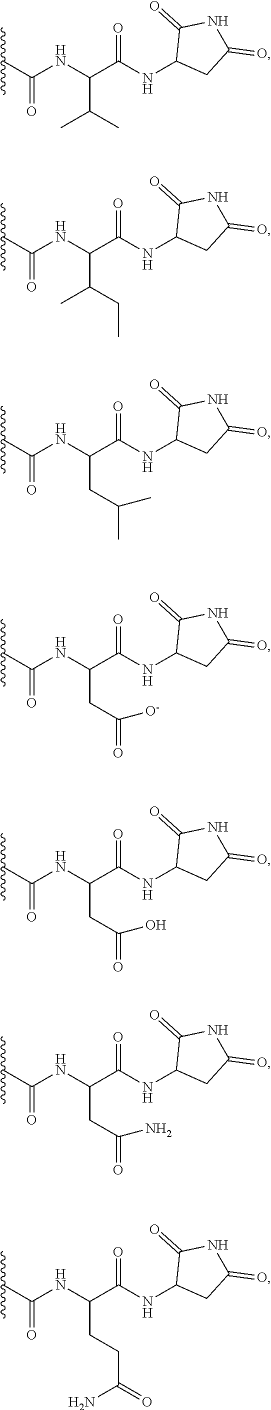

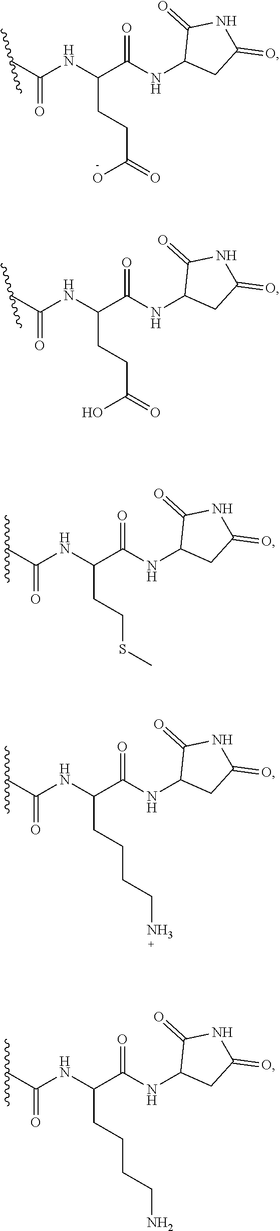 [SMILES: *C(=O)NC(CCC(=O)O)C(=O)NC1CC(=O)NC1=O ; *C(=O)NC(CCC(=O)O)C(=O)NC1CC(=O)NC1=O ; *C(=O)NC(CCSC)C(=O)NC1CC(=O)NC1=O ; *C(=O)NC(CCCCN)C(=O)NC1CC(=O)NC1=O ; *C(=O)NC(CCCCN)C(=O)NC1CC(=O)NC1=O]

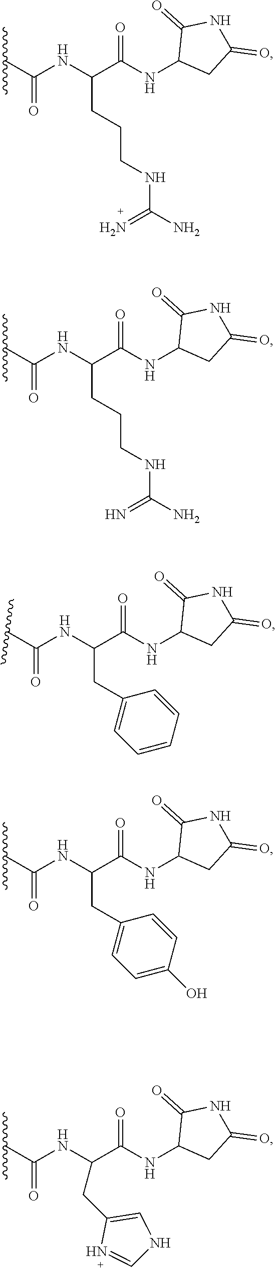 [SMILES: *C(=O)NC(CCCNC(=N)N)C(=O)NC1CC(=O)NC1=O ; *C(=O)NC(CCCNC(=N)N)C(=O)NC1CC(=O)NC1=O ; *C(=O)NC(Cc1ccccc1)C(=O)NC1CC(=O)NC1=O ; *C(=O)NC(Cc1ccc(O)cc1)C(=O)NC1CC(=O)NC1=O ; *C(=O)NC(Cc1c[nH]cn1)C(=O)NC1CC(=O)NC1=O]

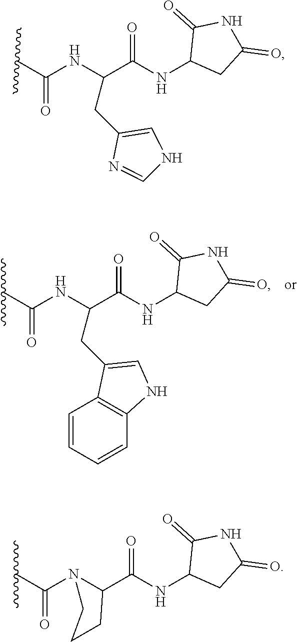 [SMILES: *C(=O)NC(Cc1c[nH]cn1)C(=O)NC1CC(=O)NC1=O ; *C(=O)NC(Cc1c[nH]c2ccccc12)C(=O)NC1CC(=O)NC1=O ; *C(=O)N1CCCC1C(=O)NC1CC(=O)NC1=O]

In some embodiments,

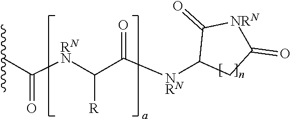

is of the formula:

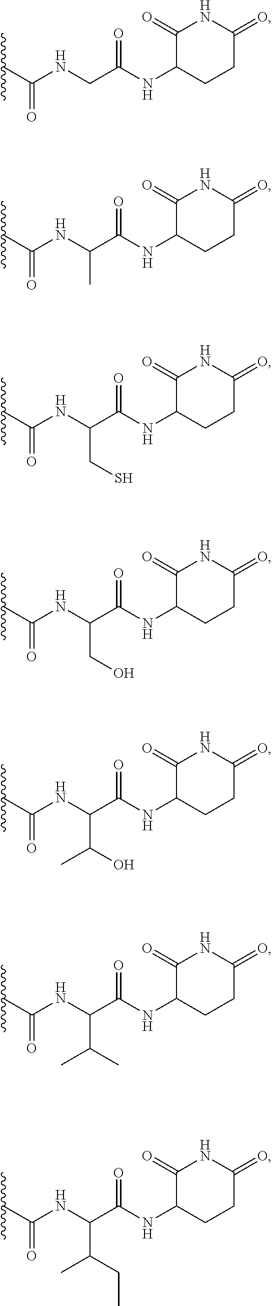

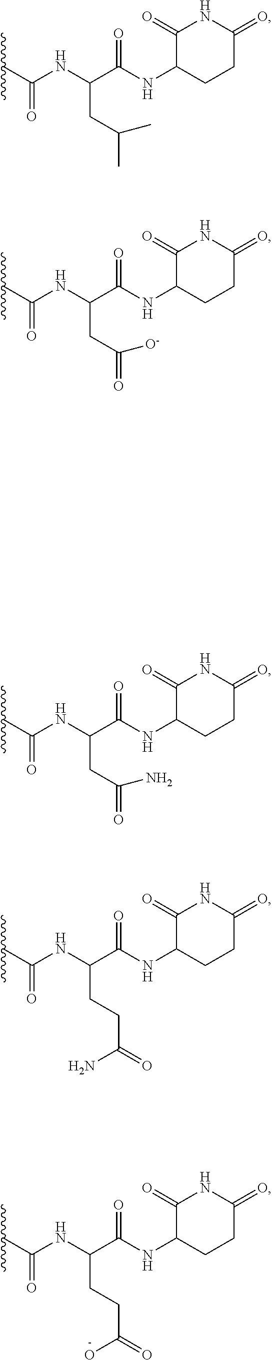 [SMILES: *C(=O)NC(CC(C)C)C(=O)NC1CCC(=O)NC1=O ; *C(=O)NC(CC(=O)[O-])C(=O)NC1CCC(=O)NC1=O ; *C(=O)NC(CC(N)=O)C(=O)NC1CCC(=O)NC1=O ; *C(=O)NC(CCC(N)=O)C(=O)NC1CCC(=O)NC1=O ; *C(=O)NC(CCC(=O)O)C(=O)NC1CCC(=O)NC1=O]

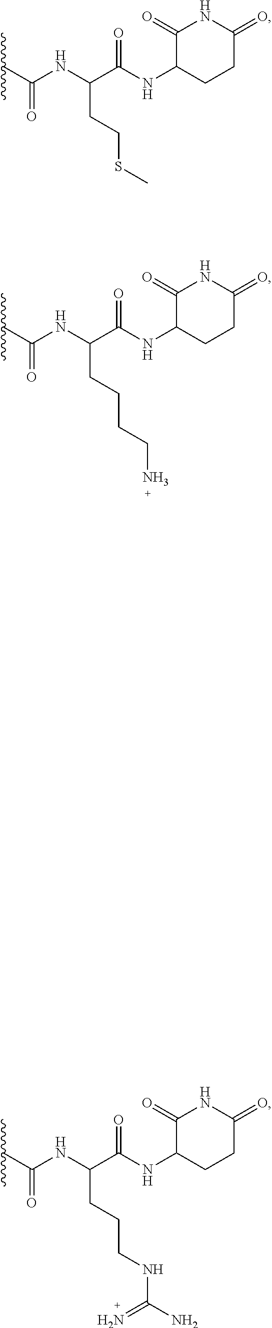 [SMILES: *C(=O)NC(CCSC)C(=O)NC1CCC(=O)NC1=O ; *C(=O)NC(CCCCN)C(=O)NC1CCC(=O)NC1=O ; *C(=O)NC(CCCNC(=N)N)C(=O)NC1CCC(=O)NC1=O]

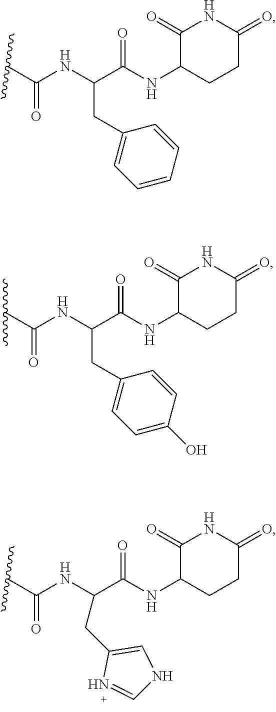 [SMILES: *C(=O)NC(Cc1ccccc1)C(=O)NC1CCC(=O)NC1=O ; *C(=O)NC(Cc1ccc(O)cc1)C(=O)NC1CCC(=O)NC1=O ; *C(=O)NC(Cc1c[nH]cn1)C(=O)NC1CCC(=O)NC1=O]

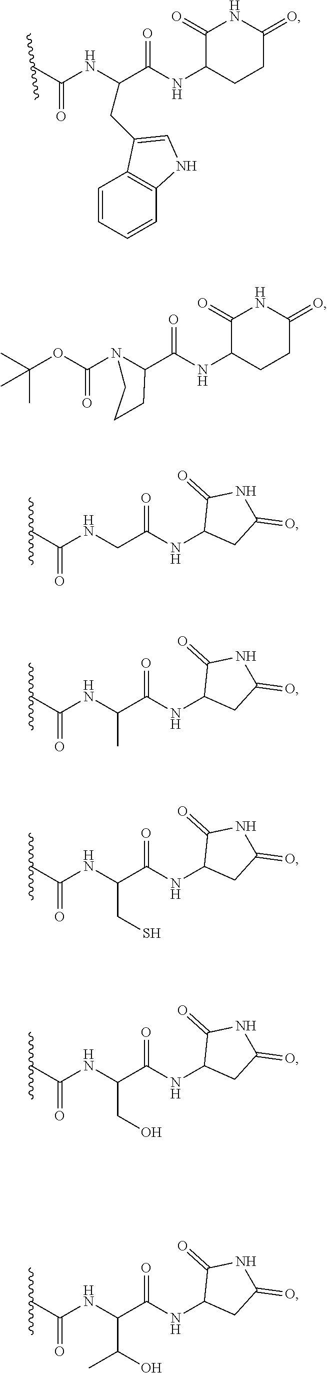

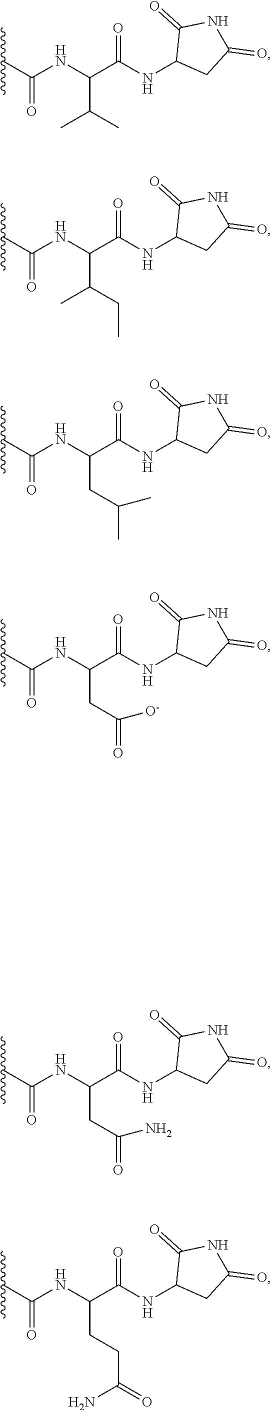 [SMILES: *C(=O)NC(C(=O)NC1CC(=O)NC1=O)C(C)C ; *C(=O)NC(C(=O)NC1CC(=O)NC1=O)C(C)CC ; *C(=O)NC(CC(C)C)C(=O)NC1CC(=O)NC1=O ; *C(=O)NC(CC(=O)[O-])C(=O)NC1CC(=O)NC1=O ; *C(=O)NC(CC(N)=O)C(=O)NC1CC(=O)NC1=O ; *C(=O)NC(CCC(N)=O)C(=O)NC1CC(=O)NC1=O]

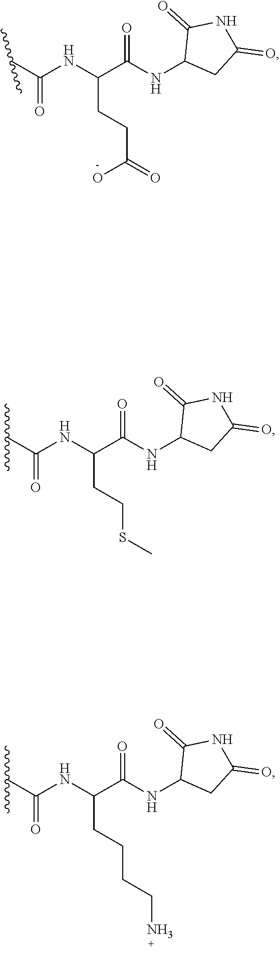 [SMILES: *C(=O)NC(CCC(=O)O)C(=O)NC1CC(=O)NC1=O ; *C(=O)NC(CCSC)C(=O)NC1CC(=O)NC1=O ; *C(=O)NC(CCCCN)C(=O)NC1CC(=O)NC1=O]

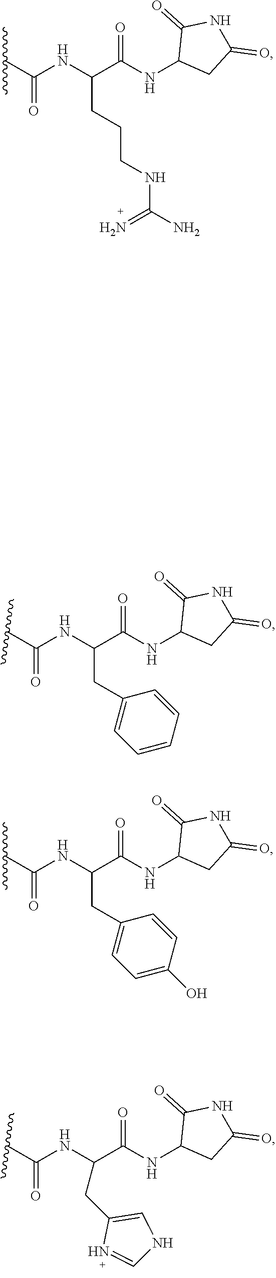 [SMILES: *C(=O)NC(CCCNC(=N)N)C(=O)NC1CC(=O)NC1=O ; *C(=O)NC(Cc1ccccc1)C(=O)NC1CC(=O)NC1=O ; *C(=O)NC(Cc1ccc(O)cc1)C(=O)NC1CC(=O)NC1=O ; *C(=O)NC(Cc1c[nH]cn1)C(=O)NC1CC(=O)NC1=O]

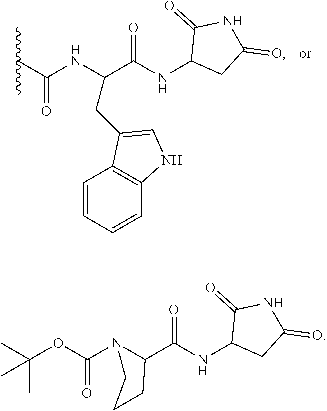 [SMILES: *C(=O)NC(Cc1c[nH]c2ccccc12)C(=O)NC1CC(=O)NC1=O ; CC(C)(C)OC(=O)N1CCCC1C(=O)NC1CC(=O)NC1=O]

In some embodiments,

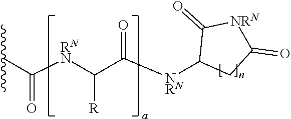

is of the formula:

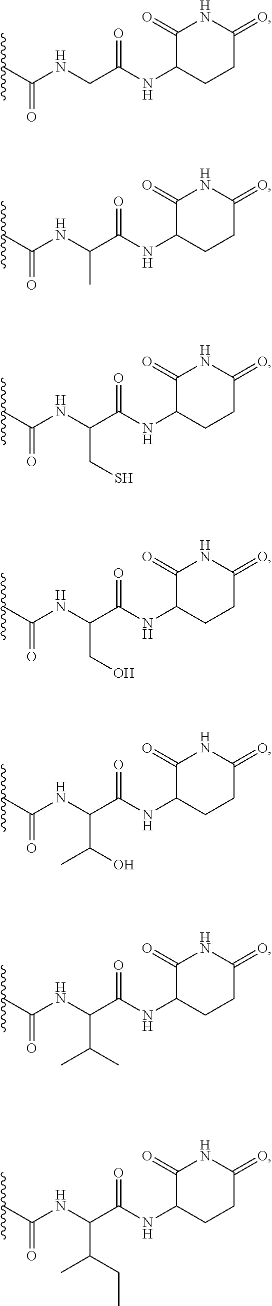

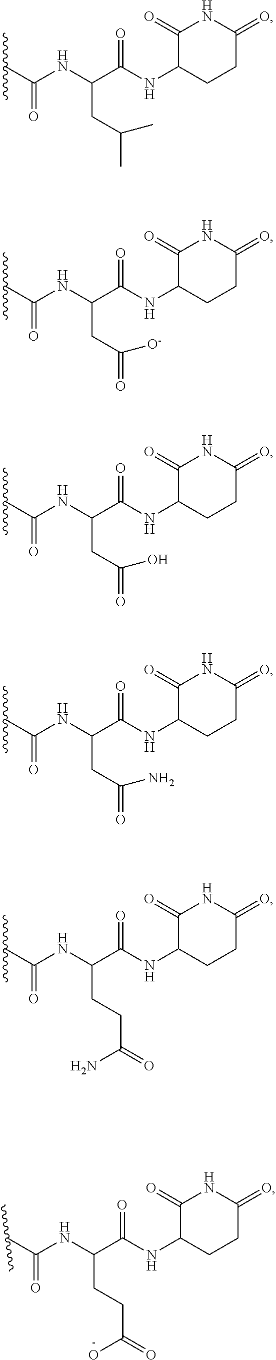 [SMILES: *C(=O)NC(CC(C)C)C(=O)NC1CCC(=O)NC1=O ; *C(=O)NC(CC(=O)[O-])C(=O)NC1CCC(=O)NC1=O ; *C(=O)NC(CC(=O)O)C(=O)NC1CCC(=O)NC1=O ; *C(=O)NC(CC(N)=O)C(=O)NC1CCC(=O)NC1=O ; *C(=O)NC(CCC(N)=O)C(=O)NC1CCC(=O)NC1=O ; *C(=O)NC(CCC(=O)O)C(=O)NC1CCC(=O)NC1=O]

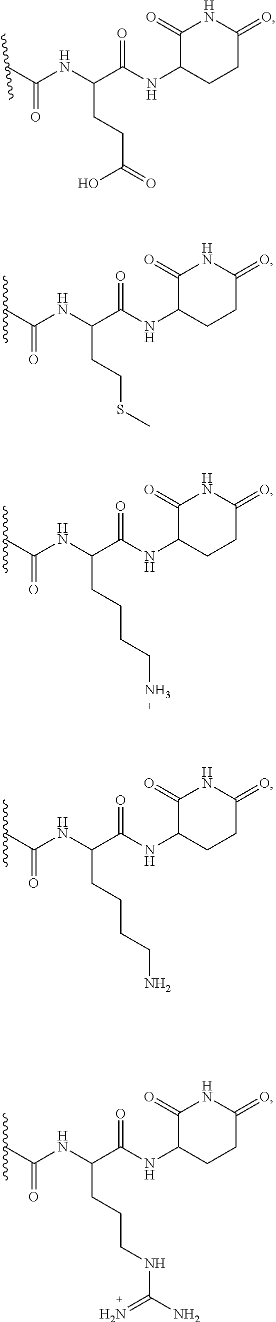 [SMILES: *C(=O)NC(CCC(=O)O)C(=O)NC1CCC(=O)NC1=O ; *C(=O)NC(CCSC)C(=O)NC1CCC(=O)NC1=O ; *C(=O)NC(CCCCN)C(=O)NC1CCC(=O)NC1=O ; *C(=O)NC(CCCCN)C(=O)NC1CCC(=O)NC1=O ; *C(=O)NC(CCCNC(=N)N)C(=O)NC1CCC(=O)NC1=O]

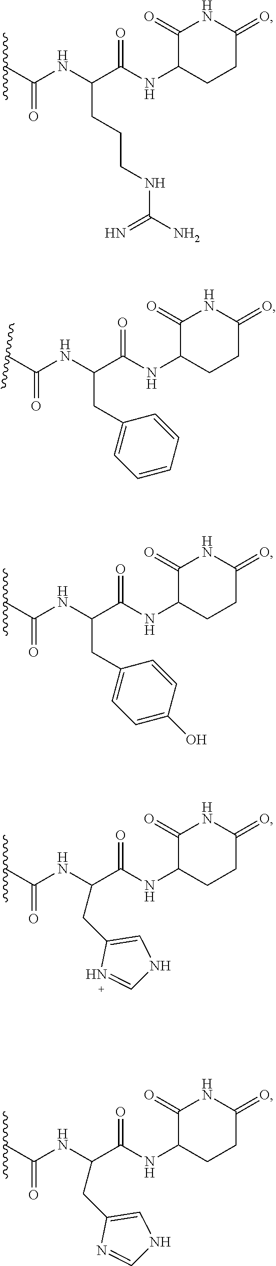 [SMILES: *C(=O)NC(CCCNC(=N)N)C(=O)NC1CCC(=O)NC1=O ; *C(=O)NC(Cc1ccccc1)C(=O)NC1CCC(=O)NC1=O ; *C(=O)NC(Cc1ccc(O)cc1)C(=O)NC1CCC(=O)NC1=O ; *C(=O)NC(Cc1c[nH]cn1)C(=O)NC1CCC(=O)NC1=O ; *C(=O)NC(Cc1c[nH]cn1)C(=O)NC1CCC(=O)NC1=O]

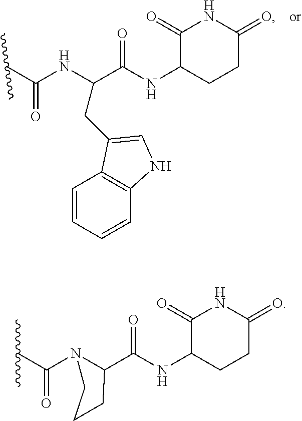 [SMILES: *C(=O)NC(Cc1c[nH]c2ccccc12)C(=O)NC1CCC(=O)NC1=O ; *C(=O)N1CCCC1C(=O)NC1CCC(=O)NC1=O]

In some embodiments,

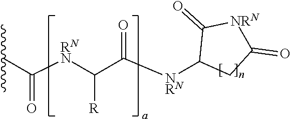

is of the formula:

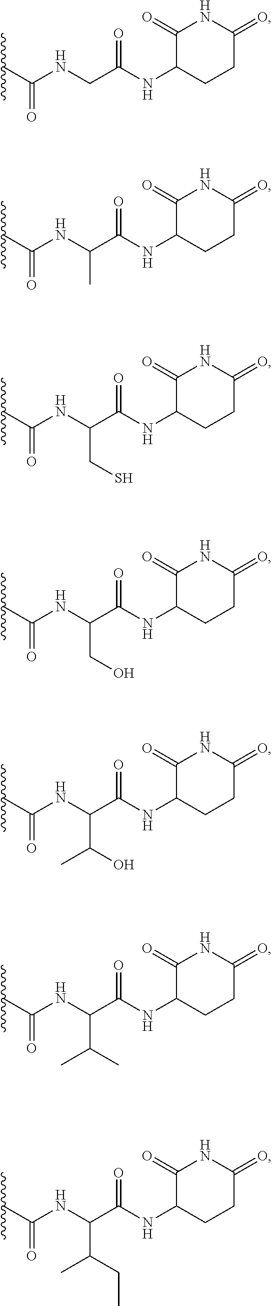

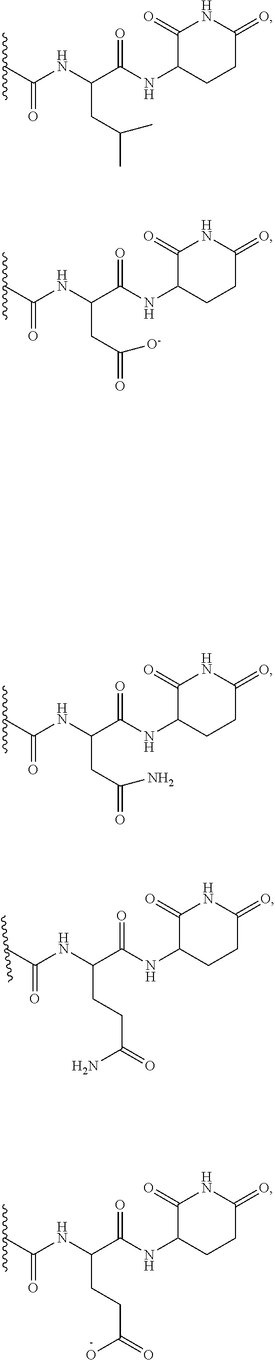 [SMILES: *C(=O)NC(CC(C)C)C(=O)NC1CCC(=O)NC1=O ; *C(=O)NC(CC(=O)[O-])C(=O)NC1CCC(=O)NC1=O ; *C(=O)NC(CC(N)=O)C(=O)NC1CCC(=O)NC1=O ; *C(=O)NC(CCC(N)=O)C(=O)NC1CCC(=O)NC1=O ; *C(=O)NC(CCC(=O)O)C(=O)NC1CCC(=O)NC1=O]

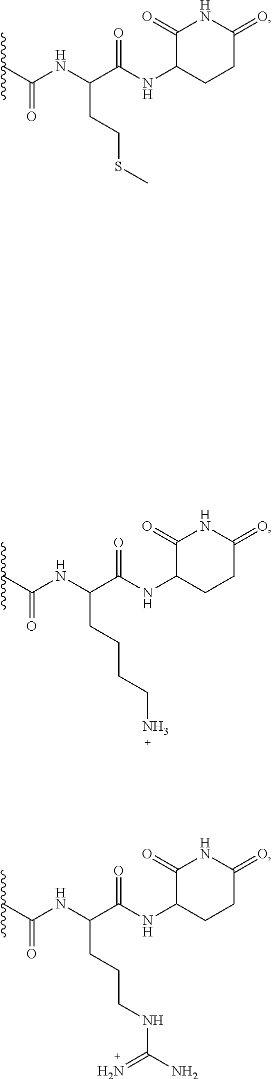 [SMILES: *C(=O)NC(CCSC)C(=O)NC1CCC(=O)NC1=O ; *C(=O)NC(CCCCN)C(=O)NC1CCC(=O)NC1=O ; *C(=O)NC(CCCNC(=N)N)C(=O)NC1CCC(=O)NC1=O]

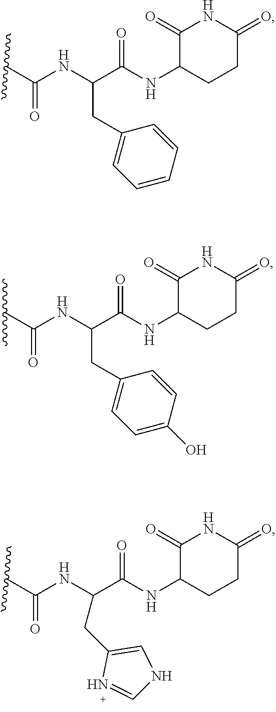 [SMILES: *C(=O)NC(Cc1ccccc1)C(=O)NC1CCC(=O)NC1=O ; *C(=O)NC(Cc1ccc(O)cc1)C(=O)NC1CCC(=O)NC1=O ; *C(=O)NC(Cc1c[nH]cn1)C(=O)NC1CCC(=O)NC1=O]

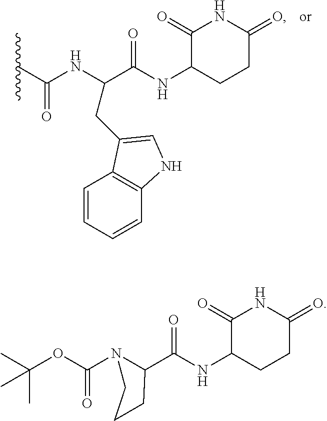 [SMILES: *C(=O)NC(Cc1c[nH]c2ccccc12)C(=O)NC1CCC(=O)NC1=O ; CC(C)(C)OC(=O)N1CCCC1C(=O)NC1CCC(=O)NC1=O]

In some embodiments,

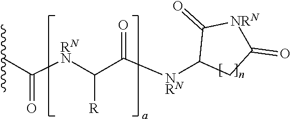

is of the formula:

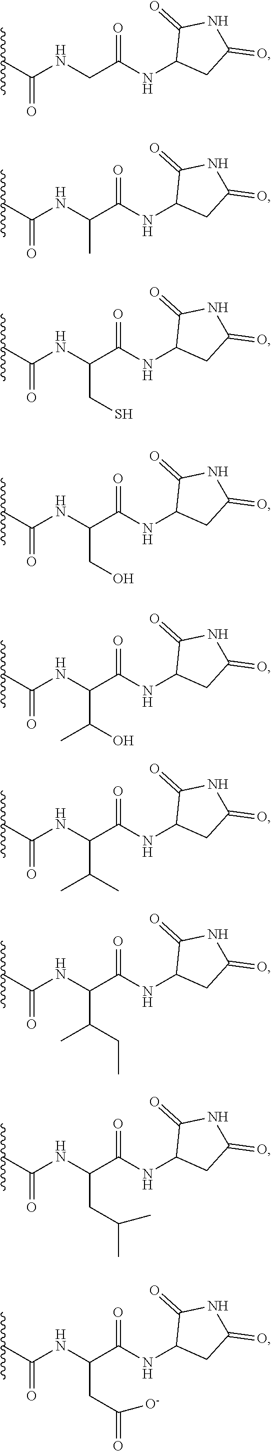

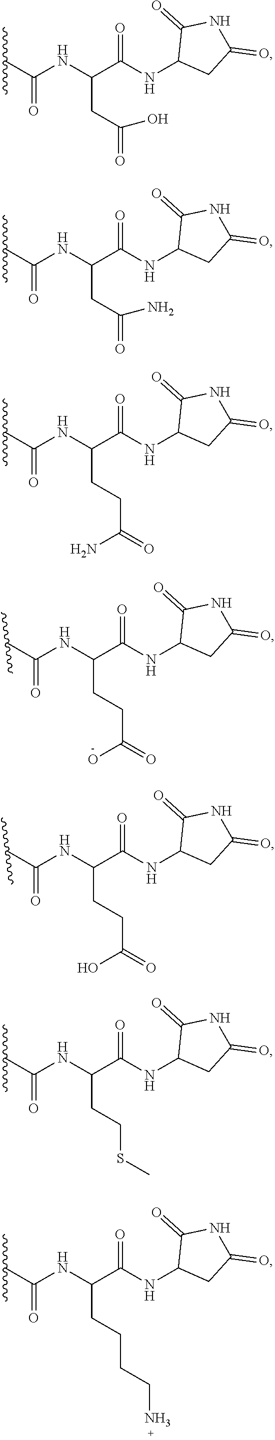

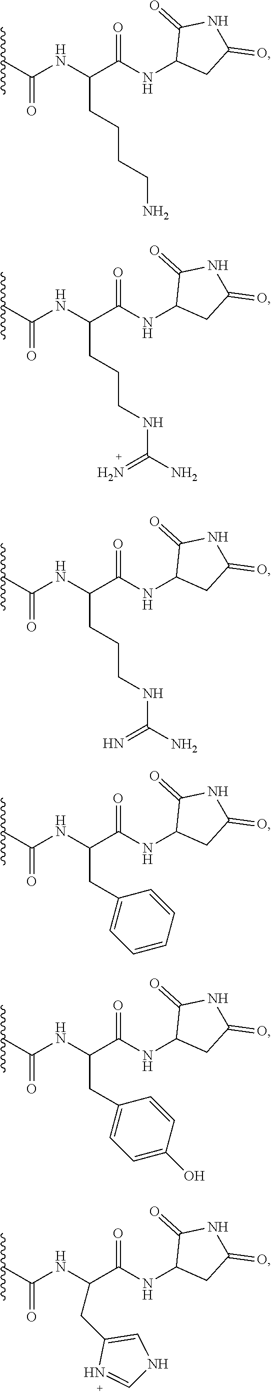

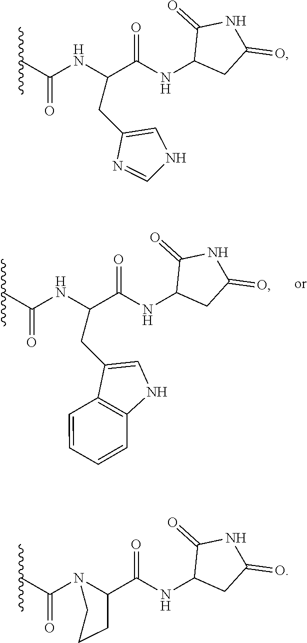 [SMILES: *C(=O)NC(Cc1c[nH]cn1)C(=O)NC1CC(=O)NC1=O ; *C(=O)NC(Cc1c[nH]c2ccccc12)C(=O)NC1CC(=O)NC1=O ; "or" ; *C(=O)N1CCCC1C(=O)NC1CC(=O)NC1=O]

In some embodiments,

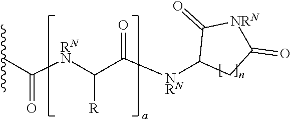

is of the formula:

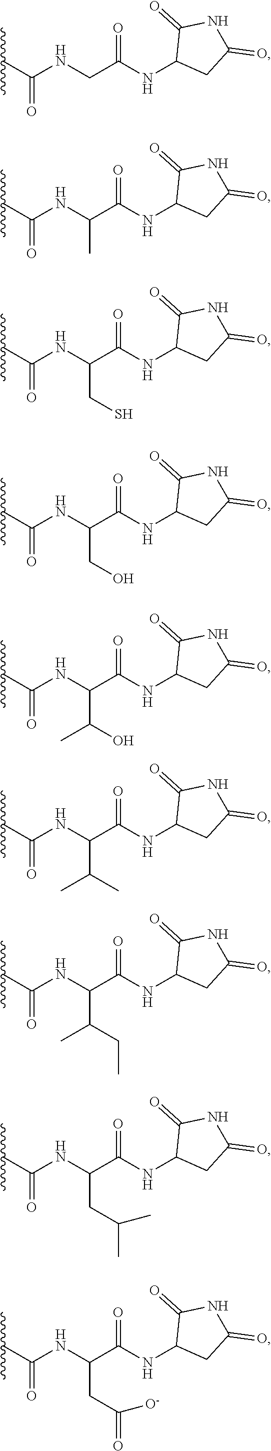

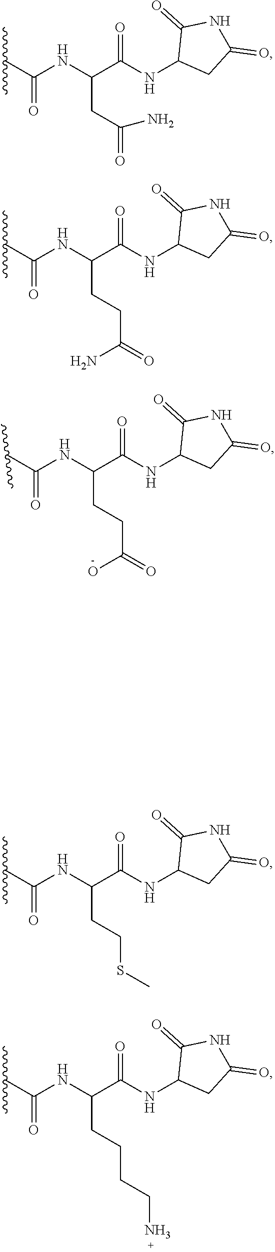

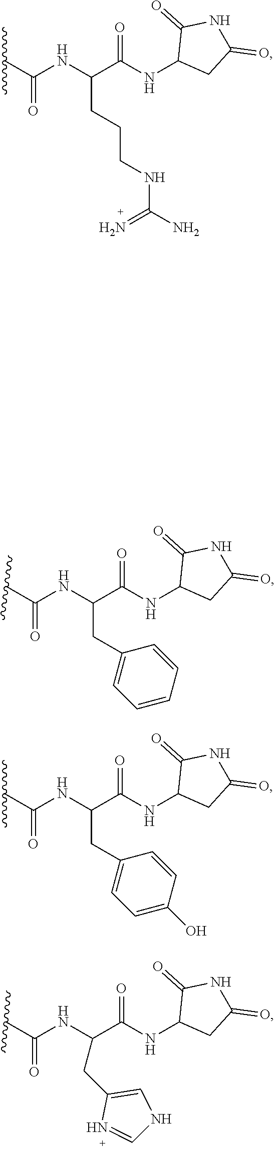

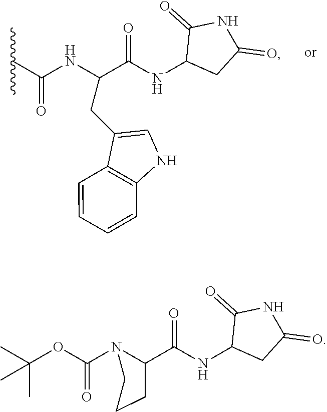 [SMILES: *C(=O)NC(Cc1c[nH]c2ccccc12)C(=O)NC1CC(=O)NC1=O ; "or" ; CC(C)(C)OC(=O)N1CCCC1C(=O)NC1CC(=O)NC1=O]

In some embodiments,

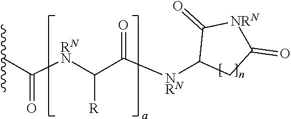

of Formula (I′) is of the formula:

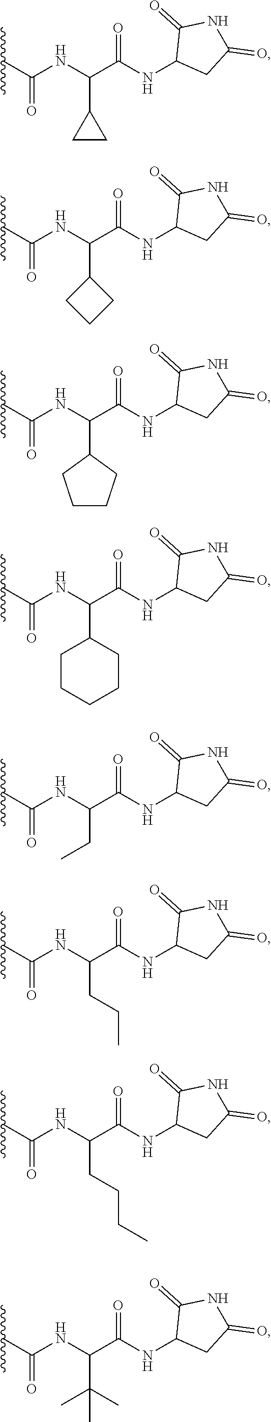

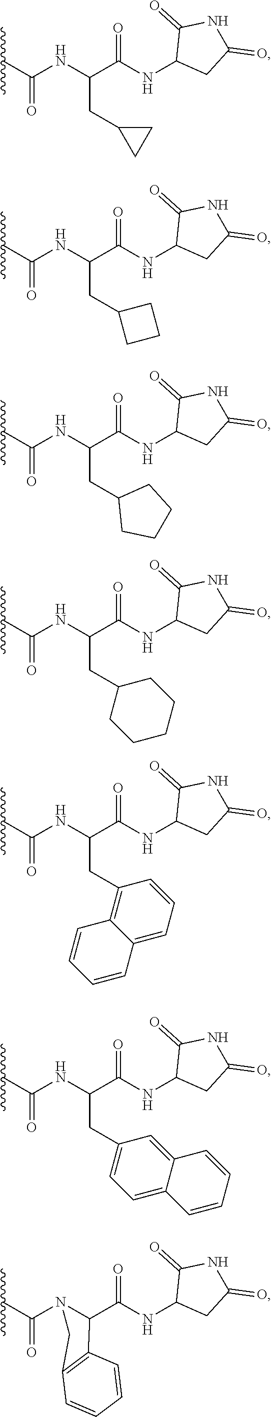

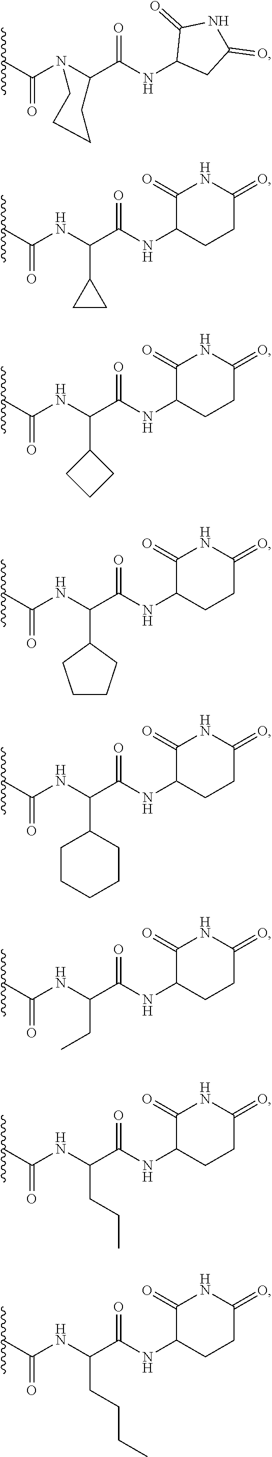

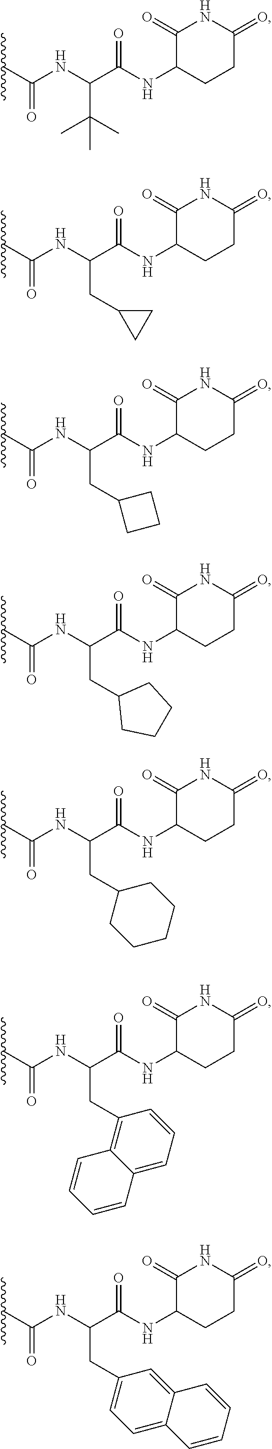

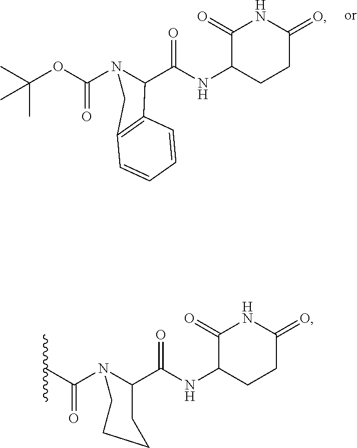 [SMILES: CC(C)(C)OC(=O)N1Cc2ccccc2C1C(=O)NC1CCC(=O)NC1=O ; "or" ; *C(=O)N1CCCCC1C(=O)NC1CCC(=O)NC1=O]

or a pharmaceutically acceptable salt or tautomer thereof. In some embodiments,

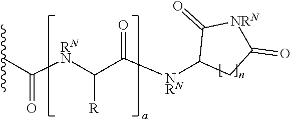

of Formula (I′) is of the formula:

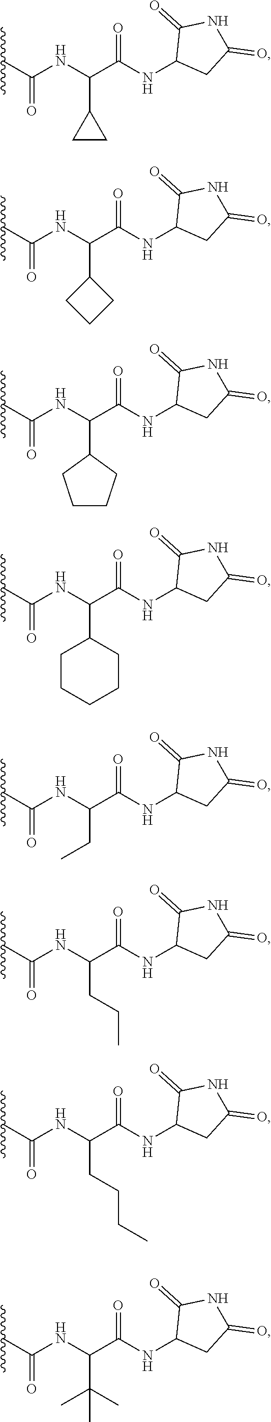

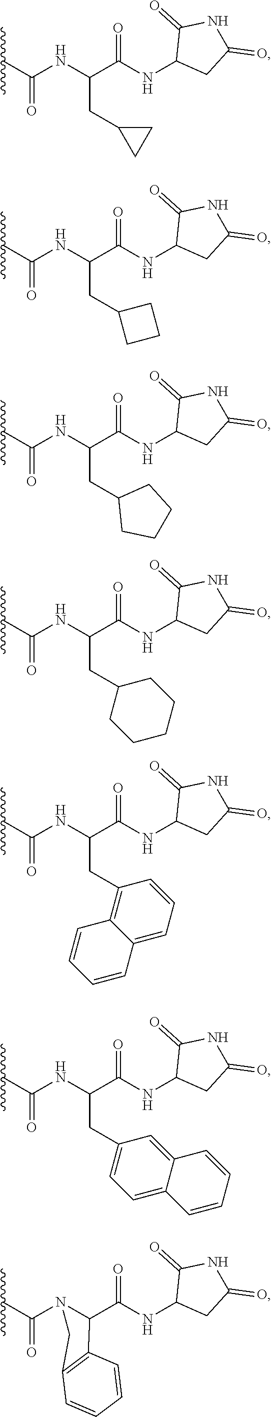

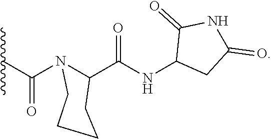 [SMILES: *C(=O)N1CCCCC1C(=O)NC1CC(=O)NC1=O]

In some embodiments,

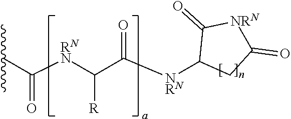

of Formula (I′) is of the formula:

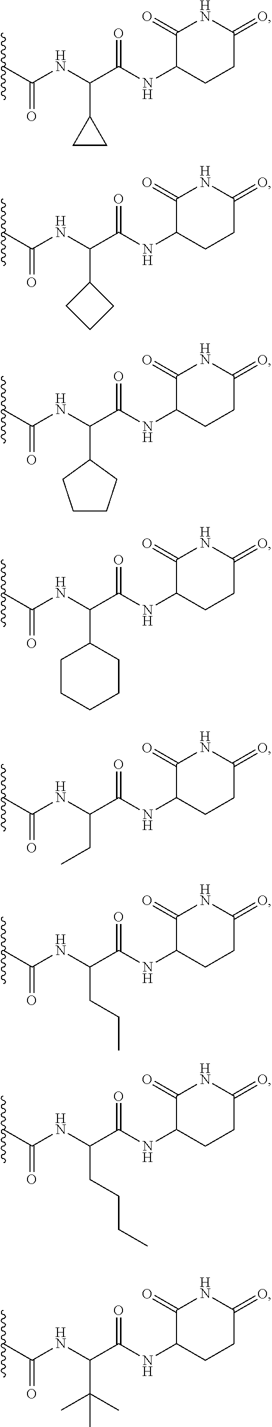

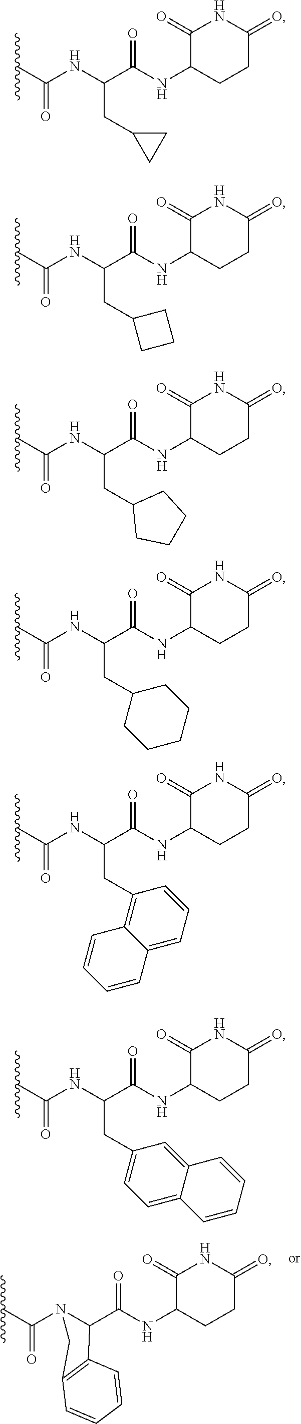

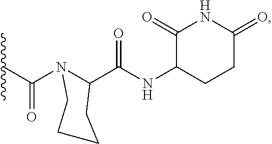 [SMILES: *C(=O)N1CCCCC1C(=O)NC1CCC(=O)NC1=O]

or a pharmaceutically acceptable salt or tautomer thereof.

In some embodiments, L¹ is a bond. In some embodiments, L¹ is optionally substituted C₁₋₂₀ alkylene, wherein optionally one or more backbone carbon atoms of the optionally substituted alkylene, optionally substituted alkenylene, optionally substituted alkynylene, optionally substituted heteroalkylene, optionally substituted heteroalkenylene, and optionally substituted heteroalkynylene are independently replaced with —O—, —S—, —NR^{A}—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene. In some embodiments, L¹ is optionally substituted C₁₋₂₀ alkylene, wherein one or more backbone carbon atoms of the optionally substituted alkylene, optionally substituted alkenylene, optionally substituted alkynylene, optionally substituted heteroalkylene, optionally substituted heteroalkenylene, and optionally substituted heteroalkynylene are independently replaced with —O—, —S—, —NR^{A}—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene. In some embodiments, L¹ is optionally substituted C₁₋₂₀ heteroalkylene, wherein optionally one or more backbone heteroatoms of the optionally substituted heteroalkylene, optionally substituted heteroalkenylene, and optionally substituted heteroalkynylene are independently replaced with —O—, —S—, —NR^{A}—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene.

In some embodiments, L¹ comprises one or more groups independently selected from —O—, —NR^{A}—, —C(═O)NR^{A}—, —NR^{A}C(═O), —C(═O)O—, —OC(═O)—, —C(═O)—,

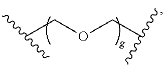

—C═C—, —C≡C—, optionally substituted piperidinylene, optionally substituted piperazinylene, optionally substituted phenylene, optionally substituted triazolylene, and optionally substituted pyrazolylene, wherein g is an integer from 1 to 10. In certain embodiments, L¹ comprises at least three groups independently selected from —O—, —NR^{A}—, —C(═O)NR^{A}—, —NR^{A}C(═O)—, —C(═O)O—, —OC(═O)—, —C(═O)—,

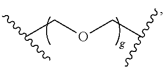

—C═C—, —C≡C—, optionally substituted piperidinylene, optionally substituted piperazinylene, optionally substituted phenylene, optionally substituted triazolylene, and optionally substituted pyrazolylene, wherein g is an integer from 1 to 10. In certain embodiments, L¹ comprises one or more groups independently selected from —O—, —NR^{A}—, —C(═O)NR^{A}—, —NR^{A}C(═O)—, —C(═O)O—, and —OC(═O)—.

In some embodiments, L¹ comprises one or more groups independently selected from —O—, —NR^{A}—, —C(═O)NR^{A}—, —NR^{A}C(═O)—, —C(═O)O—, and —OC(═O)—. In some embodiments, L¹ is C₄₋₁₆ alkylene, wherein 1, 2, or 3 backbone carbon atoms of the alkylene are independently replaced with —O—, —NR^{A}—, —C(═O)NR^{A}—, —NR^{A}C(═O)—, —C(═O)O—, or —OC(═O)—. In certain embodiments, L¹ is C₄₋₁₆ alkylene wherein 1 backbone carbon atom of the alkylene is replaced with —C(═O)NR^{A}—, —NR^{A}C(═O)—, —C(═O)O—, or —OC(═O)—.

In some embodiments, L¹ comprises one or more groups independently selected from —O—, —NR^{A}—, —C(═O)NR^{A}—, —NR^{A}C(═O), —C(═O)—, —C(═O)O—, and —OC(═O)—. In some embodiments, L¹ is C₄₋₁₆ alkylene, wherein 1, 2, or 3 backbone carbon atoms of the alkylene are independently replaced with —O—, —NR^{A}—, —C(═O)NR^{A}—, —NR^{A}C(═O)—, —C(═O)—, —C(═O)O—, or —OC(═O)—. In certain embodiments, L¹ is C₄₋₁₆ alkylene wherein 1 backbone carbon atom of the alkylene is replaced with —C(═O)NR^{A}—, —NR^{A}C(═O)—, —C(═O)—, —C(═O)O—, or —OC(═O)—.

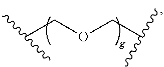

In certain embodiments, L¹ comprises wherein g is an integer from 1 to 10. In certain embodiments, g is 1, 2, 3, 4, 5, 6, 7, 8, 9, or 10. In certain embodiments, g is 2. In some embodiments, g is 3. In certain embodiments, g is 4.

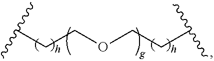

In certain embodiments, L¹ comprises wherein: g is an integer from 1 to 10; and each instance of h is independently an integer from 1 to 10, inclusive. In some embodiments, g is 2; and each instance of h is independently 1 or 2. In some embodiments, g is 3; and each instance of h is independently 1 or 2. In some embodiments, g is 4; and each instance of h is independently 1 or 2.

In some embodiments, L¹ is

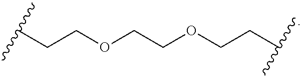 [SMILES: *CCOCCOCC*]

In certain embodiments, L¹ is optionally substituted C₄₋₁₆ alkylene. In certain embodiments, L¹ is C₄₋₁₆ alkylene. In certain embodiments, L¹ is C₄ alkylene or C₆ alkylene. In some embodiments, L¹ is optionally substituted C₄₋₁₆ alkylene, wherein 1, 2, or 3 backbone carbon atoms of the alkylene are independently replaced with —O—, —NR^{A}—, —C(═O)NR^{A}—, —NR^{A}C(═O)—, —C(═O)O—, or —OC(═O)—. In some embodiments, L¹ is C₄₋₁₆ alkylene, wherein 1, 2, or 3 backbone carbon atoms of the alkylene are independently replaced with —O—, —NR^{A}—, —C(═O)NR^{A}—, —NR^{A}C(═O)—, —C(═O)O—, or —OC(═O)—. In some embodiments, L¹ is optionally substituted C₄₋₁₆ alkylene wherein 1 backbone carbon atom of the alkylene is replaced with —C(═O)NR^{A}—, —NR^{A}C(═O)—, —C(═O)O—, or —OC(═O)—. In some embodiments, L¹ is C₄₋₁₆ alkylene wherein 1 backbone carbon atom of the alkylene is replaced with —C(═O)NR^{A}—, —NR^{A}C(═O)—, —C(═O)O—, or —OC(═O)—.

In some embodiments, L¹ is

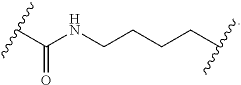 [SMILES: *CCCCNC(*)=O]

In some embodiments, L¹ is optionally substituted C₃ alkylene, wherein 1 backbone carbon atom of the alkylene is replaced with —O—. In some embodiments, L¹ is C₃ alkylene substituted with alkyl, wherein 1 backbone carbon atom of the alkylene is replaced with —O—. In some embodiments, L¹ is —O—C(CH₃)₂CH₂—. In some embodiments, L¹ is —O—C(CH₃)₂CH₂—; and B is hydrogen. In some embodiments, L¹ is optionally substituted C₂ alkylene, wherein 1 backbone carbon atom of the alkylene is replaced with —O—. In some embodiments, L¹ is C₂ alkylene substituted with aryl, wherein 1 backbone carbon atom of the alkylene is replaced with —O—. In some embodiments, L¹ is

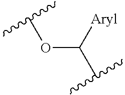

wherein aryl is selected from phenyl or fluorenyl. In some embodiments, L¹ is

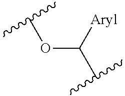

wherein aryl is selected from phenyl or fluorenyl; and B is hydrogen. In some embodiments, L¹ is optionally substituted C₁ alkylene.

In some embodiments, L¹ is —CH₂—. In some embodiments, L¹ is C₁₋₄ alkylene substituted with halogen. In some embodiments, L¹ is —CF₂—.

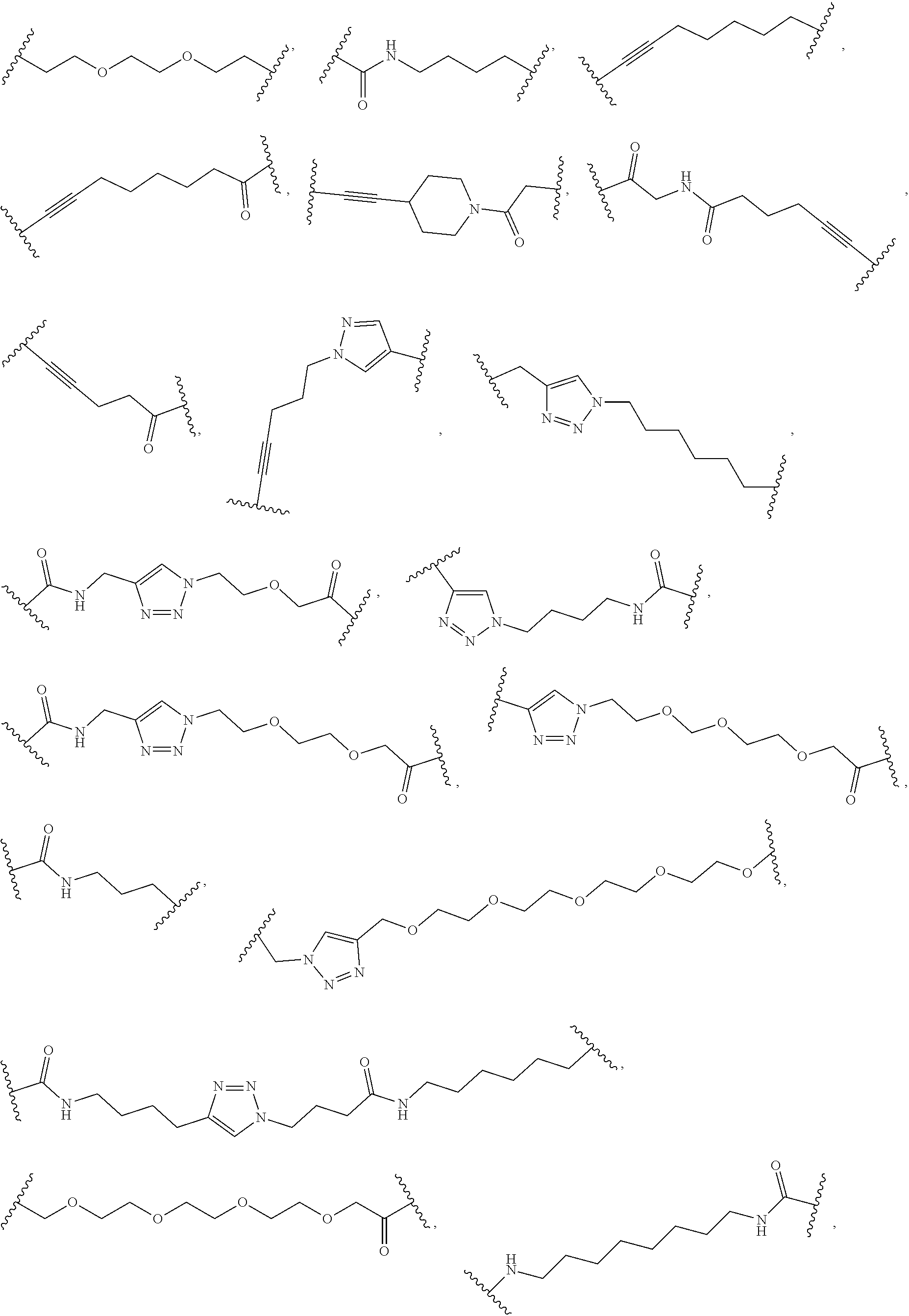

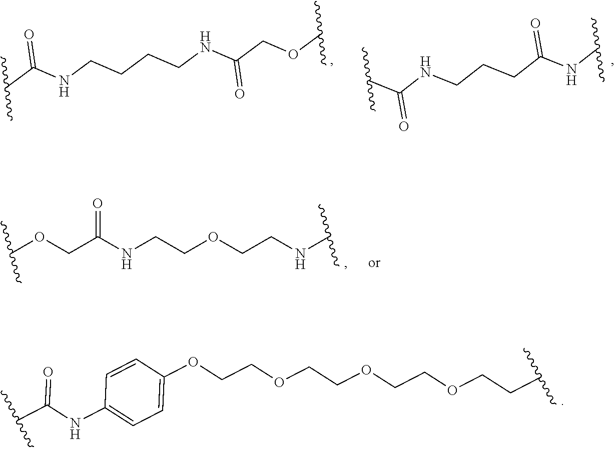 [SMILES: *OCC(=O)NCCCCNC(*)=O ; *NC(=O)CCCNC(*)=O ; *NCCOCCNC(=O)CO* ; *CCOCCOCCOCCOc1ccc(NC(*)=O)cc1]

In some embodiments, L¹ is

In some embodiments, B is hydrogen. In some embodiments, B is halogen (e.g., fluoro). In certain embodiments B is optionally substituted alkyl.

In some embodiments, B is a binder of a target. In some embodiments, B is a binder of a target wherein the target is selected from a protein (e.g., a receptor, enzyme, antibody, hormone, contractile protein, hormonal protein, structural protein, storage protein, transport protein, regulatory proteins, defensive protein), polypeptide, peptide, carbohydrate, and small molecule (e.g., signaling molecule, amino acid, cofactor). In some embodiments, the target is a protein, polypeptide, or peptide. In some embodiments, B is a small molecule, nucleic acid, or polypeptide. In some embodiments, B is a small molecule. In some embodiments, B is a binder of a protein. In some embodiments, B is a binder of any protein known in the art. In some embodiments, B is a binder of a receptor, enzyme, antibody, hormone, contractile protein, hormonal protein, structural protein, storage protein, transport protein, regulatory proteins, defensive protein. In some embodiments, B is a binder of a receptor, enzyme, antibody, hormone, or protein. In some embodiments, B is a binder of a polypeptide. In certain embodiments, B is a binder of a peptide. In some embodiments, B is a binder of a carbohydrate. In some embodiments, B is a binder of a small molecule. In some embodiments, the small molecule is selected from a signaling molecule, amino acid, and cofactor. In some embodiments, B is a binder of a target selected from the group consisting of a bromodomain, a bromodomain-containing protein, a histone methyltransferase, a kinase, a cytosolic signaling protein, a nuclear protein, a histone deacetylase, a lysine methyltransferase, a protein regulating angiogenesis, a protein regulating immune response, an aryl hydrocarbon receptor, a hormone receptor, and a transcription factor. In some embodiments, B is a binder of AKT, Anaplastic lymphoma kinase (ALK), Androgen receptor, Aryl hydrocarbon receptor, aryl hydrocarbon receptor (AHR), B-cell receptor (BCR), BCL2, BCL6, BCR-ABL, Brg/Brahma-associated factors (BAF complex), bromodomain and extraterminal (BET), Bromodomain-containing protein 2 (BRD2), Bromodomain-containing protein 3 (BRD3), Bromodomain-containing protein 4 (BRD4), Bromodomain-containing protein 7 (BRD7), Bromodomain-containing protein 9 (BRD9), Bruton's tyrosine kinase (BTK), Casein kinase 2 (CK2), Cyclin dependent kinase 4, Cyclin dependent kinase 6, Cyclin dependent kinase 8, Cyclin dependent kinase 9, c-Met, CRAPBPI and CRAPBPII, Dihydroorotate dehydrogenase (DHODH), HER2, Epidermal growth factor receptor (EGFR), ERK1, ERK2, ERRα, estrogen receptor (ER), estrogen-related receptors (ERRs), Eukaryotic translation initiation factor 4E (eIF4E), FK506 binding protein 12 (FKBP12), FMS-like tyrosine kinase 3 (FLT3), Focal adhesion kinase (FAK or PTK2), FRS2a, general control nonderepressible 5 (GCN5), HCV NS3/4A, Histone deacetylases (HDACs), Interleukin-1 receptor-associated kinase 4 (IRAK4), MetAP2, Murine double minute 2 (MDM2), Myeloid cell leukemia 1 (MCL1), NS3 protein, P300/CBP-associated factor (PCAF), p38 MAPK kinases, PCAF, GCN5, Phosphoinositide 3-kinases (P13Ks), Pirin, Poly (ADP-ribose) polymerases (PARPs), Polycomb repressive complex 2 (PRC2), PTK2/FAK, RAR, RIPK2, Rpn13, Serum/glucocorticoid-inducible protein kinase (SGK), SGK3, signal transducer and activator of transcription (STAT) proteins, Sirtuin2 (Sirt2), Smad3, SMARCA2, SMARCA4, PBRM1, TACC3, TANK-binding kinase 1 (TBK1), Tau, TBK1, TRIM24, or VHL. In some embodiments, B is a binder of cellular retinoic acid-binding proteins, dimetallohydrolase, fibroblast growth factor receptor substrate 2, lysine deacetylase, nuclear receptor, peptidyl-prolyl cis-trans isomerase, poly (ADP-ribose) polymerases, or transcriptional regulator. In some embodiments, B is a binder of a nuclear protein. In some embodiments, B is a binder of a protein regulating angiogenesis. In some embodiments, B is a binder of a protein regulating immune response. In some embodiments, B is a binder of an aryl hydrocarbon receptor.

In some embodiments B is a binder of a bromodomain. In some embodiments, the bromodomain is ASH1L, ATAD2, BAZ2B, BRD1, BRD2, BRD3, BRD4, BRD9, BRDT(1), BRPF1, CECR2, CREBBP, EP300, FALZ, GCN5L2, KIAA1240, LOC93349, PB1, PCAF, PHIP, SMARCA2, SMARCA4, SP140, TAF1, TAF1, TAF1L, TIF1, TRIM28, or WDR9(2).

In some embodiments, B is a bromodomain-containing protein 1 (BRD1) binder a bromodomain-containing protein 2 (BRD2) binder, bromodomain-containing protein 3 (BRD3) binder, or bromodomain-containing protein 4 (BRD4) binder. In some embodiments, B is a bromodomain-containing protein 4 (BRD4) binder.

In some embodiments, B is a binder of a bromodomain-containing protein. In some embodiments, B is a bromodomain-containing protein 4 (BRD4) binder. In some embodiments, B is a BET inhibitor. In certain embodiments, the bromodomain-containing protein is a bromo and extra terminal (BET) protein. In certain embodiments, the bromodomain-containing protein is a bromo and extra terminal (BET) protein, BRD2, BRD2(1), BRD2(2), BRD3, BRD3(1), BRD3(2), BRD4, BRD4(1), BRD4(2), BRDT, BRDT(1), BRDT(2), a TBP (TATA box binding protein)-associated factor protein (TAF), TAF1, TAF1L, a CREB-binding protein (CBP), or a E1A binding protein p300 (EP300).

In some embodiments, B is a binder of a histone deacetylase. In some embodiments, the histone methyltransferase is HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, and SIRT. In some embodiments, B is an HDAC inhibitor.

In some embodiments, B is a binder of a histone methyltransferase. In some embodiments, the histone methyltransferase is G9a, GLP, MLL1, MLL2, MLL3, MLL4, NSD2, PRMT1, PRMT3, PRMT4, PRMT5, PRMT6, SET1b, SET7/9, SET8, SETMAR, SMYD2, SUV39H1, or SUV39H2. In some embodiments, B is an HMT inhibitor. In some embodiments, the histone methyl transferase is a lysine methyl transferase. In some embodiments, B is a binder of a lysine methyltransferase. In some embodiments, B is a lysine methyltransferase inhibitor.

In some embodiments, B is a binder of a kinase. In some embodiments, the kinase is a tyrosine kinase, a serine/threonine kinase, a cyclin dependent kinase, or a leucine-rich repeat kinase. In some embodiments, the kinase is a cyclin dependent kinase. In some embodiments, the kinase is selected from AAK1, ABL, ACK, ACTR2, ACTR2B, AKT1, AKT2, AKT3, ALK, ALK1, ALK2, ALK4, ALK7, AMPKa1, AMPKa2, ANKRD3, ANPa, ANPb, ARAF, ARAFps, ARG, AurA, AurAps1, AurAps2, AurB, AurBps1, AurC, AXL, BARK1, BARK2, BIKE, BLK, BMPRIA, BMPRIAps1, BMPRIAps2, BMPRIB, BMPR2, BMX, BRAF, BRAFps, BRK, BRSK1, BRSK2, BTK, BUB1, BUBR1, CaMK1a, CaMK1b, CaMK1d, CaMK1g, CaMK2a, CaMK2b, CaMK2d, CaMK2g, CaMK4, CaMKK1, CaMKK2, caMLCK, CASK, CCK4, CCRK, CDC2, CDC7, CDK10, CDK11, CDK1, CDK2, CDK3, CDK4, CDK4ps, CDK5, CDK5ps, CDK6, CDK7, CDK7ps, CDK8, CDK8ps, CDK9, CDKL1, CDKL2, CDKL3, CDKL4, CDKL5, CGDps, CHED, CHK1, CHK2, CHK2ps1, CHK2ps2, CK1a, CK1a2, CK1aps1, CK1aps2, CK1aps3, CK1d, CK1e, CK1g1, CK1g2, CK1g2ps, CK1g3, CK2al, CK2al-rs, CK2a2, CLIK1, CLIKIL, CLK1, CLK2, CLK2ps, CLK3, CLK3ps, CLK4, COT, CRIK, CRK7, CSK, CTK, CYGD, CYGF, DAPK1, DAPK2, DAPK3, DCAMKL1, DCAMKL2, DCAMKL3, DDR1, DDR2, DLK, DMPK1, DMPK2, DRAK1, DRAK2, DYRKIA, DYRKIB, DYRK2, DYRK3, DYRK4, EGFR, EphA1, EphA10, EphA2, EphA3, EphA4, EphA5, EphA6, EphA7, EphA8, EphB1, EphB2, EphB3, EphB4, EphB6, Erk1, Erk2, Erk3, Erk3ps1, Erk3ps2, Erk3ps3, Erk3ps4, Erk4, Erk5, Erk7, FAK, FER, FERps, FES, FGFR1, FGFR2, FGFR3, FGFR4, FGR, FLT1, FLT1ps, FLT3, FLT4, FMS, FRK, Fused, FYN, GAK, GCK, GCN2, GCN22, GPRK4, GPRK5, GPRK6, GPRK6ps, GPRK7, GSK3A, GSK3B, Haspin, HCK, HER2/ErbB2, HER3/ErbB3, HER4/ErbB4, HH498, HIPK1, HIPK2, HIPK3, HIPK4, HPK1, HRI, HRIps, HSER, HUNK, ICK, IGF1R, IKKa, IKKb, IKKe, ILK, INSR, IRAK1, IRAK2, IRAK3, IRAK4, IRE1, IRE2, IRR, ITK, JAK1, JAK2, JAK3, JNK1, JNK2, JNK3, KDR, KHS1, KHS2, KIS, KIT, KSGCps, KSR1, KSR2, LATS1, LATS2, LCK, LIMK1, LIMK2, LIMK2ps, LKB1, LMR1, LMR2, LMR3, LOK, LRRK1, LRRK2, LTK, LYN, LZK, MAK, MAP2K1, MAP2K1ps, MAP2K2, MAP2K2ps, MAP2K3, MAP2K4, MAP2K5, MAP2K6, MAP2K7, MAP3K1, MAP3K2, MAP3K3, MAP3K4, MAP3K5, MAP3K6, MAP3K7, MAP3K8, MAPKAPK2, MAPKAPK3, MAPKAPK5, MAPKAPKps1, MARK1, MARK2, MARK3, MARK4, MARKps01, MARKps02, MARKps03, MARKps04, MARKps05, MARKps07, MARKps08, MARKps09, MARKps10, MARKps11, MARKps12, MARKps13, MARKps15, MARKps16, MARKps17, MARKps18, MARKps19, MARKps20, MARKps21, MARKps22, MARKps23, MARKps24, MARKps25, MARKps26, MARKps27, MARKps28, MARKps29, MARKps30, MAST1, MAST2, MAST3, MAST4, MASTL, MELK, MER, MET, MISR2, MLK1, MLK2, MLK3, MLK4, MLKL, MNK1, MNK1ps, MNK2, MOK, MOS, MPSK1, MPSK1ps, MRCKa, MRCKb, MRCKps, MSK1, MSK12, MSK2, MSK22, MSSK1, MST1, MST2, MST3, MST3ps, MST4, MUSK, MYO3A, MYO3B, MYT1, NDR1, NDR2, NEK1, NEK10, NEK11, NEK2, NEK2ps1, NEK2ps2, NEK2ps3, NEK3, NEK4, NEK4ps, NEK5, NEK6, NEK7, NEK8, NEK9, NIK, NIM1, NLK, NRBP1, NRBP2, NuaK1, NuaK2, Obscn, Obscn2, OSR1, p38a, p38b, p38d, p38g, p70S6K, p70S6Kb, p70S6Kps1, p70S6Kps2, PAK1, PAK2, PAK2ps, PAK3, PAK4, PAK5, PAK6, PASK, PBK, PCTAIRE1, PCTAIRE2, PCTAIRE3, PDGFRa, PDGFRb, PDK1, PEK, PFTAIRE1, PFTAIRE2, PHKg1, PHKg1ps1, PHKg1ps2, PHKg1ps3, PHKg2, PIK3R4, PIM1, PIM2, PIM3, PINK1, PITSLRE, PKACa, PKACb, PKACg, PKCa, PKCb, PKCd, PKCe, PKCg, PKCh, PKCi, PKCips, PKCt, PKCz, PKD1, PKD2, PKD3, PKG1, PKG2, PKN1, PKN2, PKN3, PKR, PLK1, PLK1ps1, PLK1ps2, PLK2, PLK3, PLK4, PRKX, PRKXps, PRKY, PRP4, PRP4ps, PRPK, PSKH1, PSKH1ps, PSKH2, PYK2, QIK, QSK, RAF1, RAF1ps, RET, RHOK, RIPK1, RIPK2, RIPK3, RNAseL, ROCK1, ROCK2, RON, ROR1, ROR2, ROS, RSK1, RSK12, RSK2, RSK22, RSK3, RSK32, RSK4, RSK42, RSKL1, RSKL2, RYK, RYKps, SAKps, SBK, SCYL1, SCYL2, SCYL2ps, SCYL3, SGK, SgK050ps, SgK069, SgK071, SgK085, SgK110, SgK196, SGK2, SgK223, SgK269, SgK288, SGK3, SgK307, SgK384ps, SgK396, SgK424, SgK493, SgK494, SgK495, SgK496, SIK (e.g., SIK1, SIK2), skMLCK, SLK, Slob, smMLCK, SNRK, SPEG, SPEG2, SRC, SRM, SRPK1, SRPK2, SRPK2ps, SSTK, STK33, STK33ps, STLK3, STLK5, STLK6, STLK6ps1, STLK6-rs, SuRTK106, SYK, TAK1, TAO1, TAO2, TAO3, TBCK, TBK1, TEC, TESK1, TESK2, TGFbR1, TGFbR2, TIE1, TIE2, TLK1, TLK1ps, TLK2, TLK2ps1, TLK2ps2, TNK1, Trad, Trb1, Trb2, Trb3, Trio, TRKA, TRKB, TRKC, TSSK1, TSSK2, TSSK3, TSSK4, TSSKps1, TSSKps2, TTBK1, TTBK2, TTK, TTN, TXK, TYK2, TYK22, TYRO3, TYRO3ps, ULK1, ULK2, ULK3, ULK4, VACAMKL, VRK1, VRK2, VRK3, VRK3ps, Weel, WeelB, WeelBps, Weelps1, Weelps2, Wnk1, Wnk2, Wnk3, Wnk4, YANK1, YANK2, YANK3, YES, YESps, YSK1, ZAK, ZAP70, ZC1/HGK, ZC2/TNIK, ZC3/MINK, and ZC4/NRK. In some embodiments, B is a kinase inhibitor. In some embodiments, B is a binder of cyclin kinase dependent kinase. In some embodiments, the kinase is a cyclin dependent kinase 1 (CDK1), cyclin dependent kinase 2 (CDK2), cyclin dependent kinase 3 (CDK3), cyclin dependent kinase 4 (CDK4), cyclin dependent kinase 5 (CDK5), cyclin dependent kinase 6 (CDK6), cyclin dependent kinase 7 (CDK7), cyclin dependent kinase 8 (CDK8), cyclin dependent kinase 9 (CDK9), cyclin dependent kinase 10 (CDK10), or cyclin dependent kinase 11 (CDK11). In some embodiments, B is a cyclin dependent kinase binder.

In some embodiments, the cyclic kinase inhibitor/binder is a cyclin dependent kinase 1 (CDK1) binder, cyclin dependent kinase 2 (CDK2) binder, cyclin dependent kinase 3 (CDK3) binder, cyclin dependent kinase 4 (CDK4) binder, cyclin dependent kinase 5 (CDK5) binder, cyclin dependent kinase 6 (CDK6) binder, cyclin dependent kinase 7 (CDK7) binder, cyclin dependent kinase 8 (CDK8) binder, cyclin dependent kinase 9 (CDK9) binder, cyclin dependent kinase 10 (CDK10) binder, or cyclin dependent kinase 11 (CDK11). In some embodiments, the cyclic kinase inhibitor/binder is a cyclin dependent kinase 4 (CDK4) binder or cyclin dependent kinase 6 (CDK6) binder. In some embodiments, the cyclic kinase inhibitor/binder is a cyclin dependent kinase 4 (CDK4) binder. In some embodiments, the cyclic kinase inhibitor/binder is a cyclin dependent kinase 6 (CDK6) binder. In some embodiments, B is palbociclib. In certain embodiments, B is of the formula:

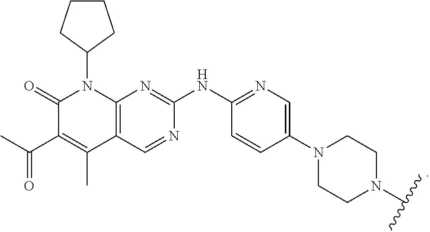 [SMILES: *N1CCN(c2ccc(Nc3ncc4c(C)c(C(C)=O)c(=O)n(C5CCCC5)c4n3)nc2)CC1]

In some embodiments, B is a binder of a cytosolic signaling protein. In some embodiments, the cytosolic signaling protein is FKBP. In some embodiments, the cytosolic signaling protein is FKBP12.

In some embodiments, B is a binder of a hormone receptor. In some embodiments, the hormone receptor is an estrogen receptor, an androgen receptor, or a glucocorticoid receptor.

In some embodiments, B is a binder of a transcription factor. In some embodiments, the transcription factor is SMARCA4, SMARCA2, or TRIM24. In some embodiments, the transcription factor is selected from AC008770.3, AC023509.3, AC092835.1, AC138696.1, ADNP, ADNP2, AEBP1, AEBP2, AHCTF1, AHDC1, AHR, AHRR, AIRE, AKAP8, AKAP8L, AKNA, ALX1, ALX3, ALX4, ANHX, ANKZF1, AR, ARGFX, ARHGAP35, ARID2, ARID3A, ARID3B, ARID3C, ARIDSA, ARIDSB, ARNT, ARNT2, ARNTL, ARNTL2, ARX, ASCL1, ASCL2, ASCL3, ASCL4, ASCL5, ASH1L, ATF1, ATF2, ATF3, ATF4, ATF5, ATF6, ATF6B, ATF7, ATMIN, ATOH1, ATOH7, ATOH8, BACH1, BACH2, BARHL1, BARHL2, BARX1, BARX2, BATF, BATF2, BATF3, BAZ2A, BAZ2B, BBX, BCL11A, BCL11B, BCL6, BCL6B, BHLHA15, BHLHA9, BHLHE22, BHLHE23, BHLHE40, BHLHE41, BNC1, BNC2, BORCS-MEF2B, BPTF, BRF2, BSX, C11orf95, CAMTA1, CAMTA2, CARF, CASZ1, CBX2, CC2D1A, CCDC169-SOHLH2, CCDC17, CDC5L, CDX1, CDX2, CDX4, CEBPA, CEBPB, CEBPD, CEBPE, CEBPG, CEBPZ, CENPA, CENPB, CENPBD1, CENPS, CENPT, CENPX, CGGBP1, CHAMP1, CHCHD3, CIC, CLOCK, CPEB1, CPXCR1, CREB1, CREB3, CREB3L1, CREB3L2, CREB3L3, CREB3L4, CREB5, CREBL2, CREBZF, CREM, CRX, CSRNP1, CSRNP2, CSRNP3, CTCF, CTCFL, CUX1, CUX2, CXXC1, CXXC4, CXXC5, DACH1, DACH2, DBP, DBX1, DBX2, DDIT3, DEAF1, DLX1, DLX2, DLX3, DLX4, DLX5, DLX6, DMBX1, DMRT1, DMRT2, DMRT3, DMRTA1, DMRTA2, DMRTB1, DMRTC2, DMTF1, DNMT1, DNTTIP1, DOTIL, DPF1, DPF3, DPRX, DR1, DRAP1, DRGX, DUX1, DUX3, DUX4, DUXA, DZIP1, E2F1, E2F2, E2F3, E2F4, E2F5, E2F6, E2F7, E2F8, E4F1, EBF1, EBF2, EBF3, EBF4, EEA1, EGR1, EGR2, EGR3, EGR4, EHF, ELF1, ELF2, ELF3, ELF4, ELF5, ELK1, ELK3, ELK4, EMX1, EMX2, EN1, EN2, EOMES, EPAS1, ERF, ERG, ESR1, ESR2, ESRRA, ESRRB, ESRRG, ESX1, ETS1, ETS2, ETV1, ETV2, ETV3, ETV3L, ETV4, ETV5, ETV6, ETV7, EVX1, EVX2, FAM170A, FAM200B, FBXL19, FERD3L, FEV, FEZF1, FEZF2, FIGLA, FIZ1, FLI1, FLYWCH1, FOS, FOSB, FOSL1, FOSL2, FOXA1, FOXA2, FOXA3, FOXB1, FOXB2, FOXC1, FOXC2, FOXD1, FOXD2, FOXD3, FOXD4, FOXD4L1, FOXD4L3, FOXD4L4, FOXD4L5, FOXD4L6, FOXE1, FOXE3, FOXF1, FOXF2, FOXG1, FOXH1, FOXI1, FOXI2, FOXI3, FOXJ1, FOXJ2, FOXJ3, FOXK1, FOXK2, FOXL1, FOXL2, FOXM1, FOXN1, FOXN2, FOXN3, FOXN4, FOXO1, FOXO3, FOXO4, FOX06, FOXP1, FOXP2, FOXP3, FOXP4, FOXQ1, FOXR1, FOXR2, FOXS1, GABPA, GATA1, GATA2, GATA3, GATA4, GATA5, GATA6, GATAD2A, GATAD2B, GBX1, GBX2, GCM1, GCM2, GFI1, GFIIB, GLI1, GLI2, GLI3, GLI4, GLIS1, GLIS2, GLIS3, GLMP, GLYR1, GMEB1, GMEB2, GPBP1, GPBP1L1, GRHL1, GRHL2, GRHL3, GSC, GSC2, GSX1, GSX2, GTF2B, GTF21, GTF2IRD1, GTF2IRD2, GTF2IRD2B, GTF3A, GZF1, HAND1, HAND2, HBP1, HDX, HELT, HES1, HES2, HES3, HES4, HES5, HES6, HES7, HESX1, HEY1, HEY2, HEYL, HHEX, HIC1, HIC2, HIFIA, HIF3A, HINFP, HIVEP1, HIVEP2, HIVEP3, HKR1, HLF, HLX, HMBOX1, HMG20A, HMG20B, HMGA1, HMGA2, HMGN3, HMX1, HMX2, HMX3, HNFIA, HNFIB, HNF4A, HNF4G, HOMEZ, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HSF1, HSF2, HSF4, HSF5, HSFX1, HSFX2, HSFY1, HSFY2, IKZF1, IKZF2, IKZF3, IKZF4, IKZF5, INSM1, INSM2, IRF1, IRF2, IRF3, IRF4, IRF5, IRF6, IRF7, IRF8, IRF9, IR^{X1}, IRX2, IRX3, IRX4, IRX5, IRX6, ISL1, ISL2, ISX, JAZF1, JDP2, JRK, JRKL, JUN, JUNB, JUND, KAT7, KCMF1, KCNIP3, KDM2A, KDM2B, KDM5B, KIN, KLF1, KLF10, KLF11, KLF12, KLF13, KLF14, KLF15, KLF16, KLF17, KLF2, KLF3, KLF4, KLF5, KLF6, KLF7, KLF8, KLF9, KMT2A, KMT2B, L3MBTL1, L3MBTL3, L3MBTL4, LBX1, LBX2, LCOR, LCORL, LEF1, LEUTX, LHX1, LHX2, LHX3, LHX4, LHX5, LHX6, LHX8, LHX9, LIN28A, LIN28B, LIN54, LMXIA, LMXIB, LTF, LYL1, MAF, MAFA, MAFB, MAFF, MAFG, MAFK, MAX, MAZ, MBD1, MBD2, MBD3, MBD4, MBD6, MBNL2, MECOM, MECP2, MEF2A, MEF2B, MEF2C, MEF2D, MEIS1, MEIS2, MEIS3, MEOX1, MEOX2, MESP1, MESP2, MGA, MITF, MIXL1, MKX, MLX, MLXIP, MLXIPL, MNT, MNX1, MSANTD1, MSANTD3, MSANTD4, MSC, MSGN1, MSX1, MSX2, MTERF1, MTERF2, MTERF3, MTERF4, MTF1, MTF2, MXD1, MXD3, MXD4, MXI1, MYB, MYBL1, MYBL2, MYC, MYCL, MYCN, MYF5, MYF6, MYNN, MYOD1, MYOG, MYPOP, MYRF, MYRFL, MYSM1, MYT1, MYTIL, MZF1, NACC2, NAIF1, NANOG, NANOGNB, NANOGP8, NCOA1, NCOA2, NCOA3, NEUROD1, NEUROD2, NEUROD4, NEUROD6, NEUROG1, NEUROG2, NEUROG3, NFAT5, NFATC1, NFATC2, NFATC3, NFATC4, NFE2, NFE2L1, NFE2L2, NFE2L3, NFE4, NFIA, NFIB, NFIC, NFIL3, NFIX, NFκB1, NFκB2, NFX1, NFXL1, NFYA, NFYB, NFYC, NHLH1, NHLH2, NKRF, NKX1-1, NKX1-2, NKX2-1, NKX2-2, NKX2-3, NKX2-4, NKX2-5, NKX2-6, NKX2-8, NKX3-1, NKX3-2, NKX6-1, NKX6-2, NKX6-3, NME2, NOBOX, NOTO, NPAS1, NPAS2, NPAS3, NPAS4, NROB1, NRID1, NRID2, NR1H2, NR1H3, NR1H4, NR112, NR113, NR2C₁, NR2C₂, NR2E1, NR2E3, NR2F1, NR2F2, NR2F6, NR3C₁, NR3C₂, NR4A1, NR4A2, NR4A3, NR5A1, NR5A2, NR6A1, NRF1, NRL, OLIG1, OLIG2, OLIG3, ONECUT1, ONECUT2, ONECUT3, OSR1, OSR2, OTP, OTX1, OTX2, OVOL1, OVOL2, OVOL3, PA2G4, PATZ1, PAX1, PAX2, PAX3, PAX4, PAX5, PAX6, PAX7, PAX8, PAX9, PBX1, PBX2, PBX3, PBX4, PCGF2, PCGF6, PDX1, PEG3, PGR, PHF1, PHF19, PHF20, PHF21A, PHOX2A, PHOX2B, PIN1, PITX1, PITX2, PITX3, PKNOX1, PKNOX2, PLAG1, PLAGL1, PLAGL2, PLSCR1, POGK, POUIF1, POU2AF1, POU2F1, POU2F2, POU2F3, POU3F1, POU3F2, POU3F3, POU3F4, POU4F1, POU4F2, POU4F3, POU5F1, POUSFIB, POU5F2, POU6F1, POU6F2, PPARA, PPARD, PPARG, PRDM1, PRDM10, PRDM12, PRDM13, PRDM14, PRDM15, PRDM16, PRDM2, PRDM4, PRDM5, PRDM6, PRDM8, PRDM9, PREB, PRMT3, PROP1, PROX1, PROX2, PRR12, PRR^{X1}, PRRX2, PTFIA, PURA, PURB, PURG, RAG1, RARA, RARB, RARG, RAX, RAX2, RBAK, RBCK1, RBPJ, RBPJL, RBSN, REL, RELA, RELB, REPIN1, REST, REXO4, RFX1, RFX2, RFX3, RFX4, RFX5, RFX6, RFX7, RFX8, RHOXF1, RHOXF2, RHOXF2B, RLF, RORA, RORB, RORC, RREB1, RUNX1, RUNX2, RUNX3, RXRA, RXRB, RXRG, SAFB, SAFB2, SALL1, SALL2, SALL3, SALL4, SATB1, SATB2, SCMH1, SCML4, SCRT1, SCRT2, SCX, SEBOX, SETBP1, SETDB1, SETDB2, SGSM2, SHOX, SHOX2, SIM1, SIM2, SIX1, SIX2, SIX3, SIX4, SIX5, SIX6, SK1, SKIL, SKOR1, SKOR2, SLC2A4RG, SMAD1, SMAD3, SMAD4, SMAD5, SMAD9, SMARCA4, SMARCA2, SMYD3, SNAI1, SNAI2, SNAI3, SNAPC2, SNAPC4, SNAPC5, SOHLH1, SOHLH2, SON, SOX1, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP100, SP110, SP140, SP140L, SP2, SP3, SP4, SP5, SP6, SP7, SP8, SP9, SPDEF, SPEN, SPI1, SPIB, SPIC, SPZ1, SRCAP, SREBF1, SREBF2, SRF, SRY, ST18, STAT1, STAT2, STAT3, STAT4, STATSA, STA5B, STT6, T, TAL1, TAL2, TBP, TBPL1, TBPL2, TBR1, TBX1, TBX10, TBX15, TBX18, TBX19, TBX2, TBX20, TBX21, TBX22, TBX3, TBX4, TBX5, TBX6, TCF12, TCF15, TCF20, TCF21, TCF23, TCF24, TCF3, TCF4, TCF7, TCF7L1, TCF7L2, TCFL5, TEAD1, TEAD2, TEAD3, TEAD4, TEF, TERB1, TERF1, TERF2, TET1, TET2, TET3, TFAP2A, TFAP2B, TFAP2C, TFAP2D, TFAP2E, TFAP4, TFCP2, TFCP2L1, TFDP1, TFDP2, TFDP3, TFE3, TFEB, TFEC, TGIF1, TGIF2, TGIF2LX, TGIF2LY, THAP1, THAP10, THAP11, THAP12, THAP2, THAP3, THAP4, THAP5, THAP6, THAP7, THAP8, THAP9, THRA, THRB, THYN1, TIGD1, TIGD2, TIGD3, TIGD4, TIGD5, TIGD6, TIGD7, TLX1, TLX2, TLX3, TMF1, TOPORS, TP53, TP63, TP73, TPR^{X1}, TRAFD1, TRERF1, TRPS1, TRIM24, TSC22D1, TSHZ1, TSHZ2, TSHZ3, TTF1, TWIST1, TWIST, UBP1, UNCX, USF1, USF2, USF3, VAX1, VAX2, VDR, VENTX, VEZF1, VSX1, VSX2, WIZ, WT1, XBP1, XPA, YBX1, YBX2, YBX3, YY1, YY2, ZBED1, ZBED2, ZBED3, ZBED4, ZBED5, ZBED6, ZBED9, ZBTB1, ZBTB10, ZBTB11, ZBTB12, ZBTB14, ZBTB16, ZBTB17, ZBTB18, ZBTB2, ZBTB20, ZBTB21, ZBTB22, ZBTB24, ZBTB25, ZBTB26, ZBTB3, ZBTB32, ZBTB33, ZBTB34, ZBTB37, ZBTB38, ZBTB39, ZBTB4, ZBTB40, ZBTB41, ZBTB42, ZBTB43, ZBTB44, ZBTB45, ZBTB46, ZBTB47, ZBTB48, ZBTB49, ZBTB5, ZBTB6, ZBTB7A, ZBTB7B, ZBTB7C, ZBTB8A, ZBTB8B, ZBTB9, ZC3H8, ZEB1, ZEB2, ZFAT, ZFHX2, ZFHX3, ZFHX4, ZFP1, ZFP14, ZFP2, ZFP28, ZFP3, ZFP30, ZFP37, ZFP41, ZFP42, ZFP57, ZFP62, ZFP64, ZFP69, ZFP69B, ZFP82, ZFP90, ZFP91, ZFP92, ZFPM1, ZFPM2, ZFX, ZFY, ZGLP1, ZGPAT, ZHX1, ZHX2, ZHX3, ZIC1, ZIC2, ZIC3, ZIC4, ZIC5, ZIK1, ZIM2, ZIM3, ZKSCAN1, ZKSCAN2, ZKSCAN3, ZKSCAN4, ZKSCAN5, ZKSCAN7, ZKSCAN8, ZMAT1, ZMAT4, ZNF10, ZNF100, ZNF101, ZNF107, ZNF112, ZNF114, ZNF117, ZNF12, ZNF121, ZNF124, ZNF131, ZNF132, ZNF133, ZNF134, ZNF135, ZNF136, ZNF138, ZNF14, ZNF140, ZNF141, ZNF142, ZNF143, ZNF146, ZNF148, ZNF154, ZNF155, ZNF157, ZNF16, ZNF160, ZNF165, ZNF169, ZNF17, ZNF174, ZNF175, ZNF177, ZNF18, ZNF180, ZNF181, ZNF182, ZNF184, ZNF189, ZNF19, ZNF195, ZNF197, ZNF2, ZNF20, ZNF200, ZNF202, ZNF205, ZNF207, ZNF208, ZNF211, ZNF212, ZNF213, ZNF214, ZNF215, ZNF217, ZNF219, ZNF22, ZNF221, ZNF222, ZNF223, ZNF224, ZNF225, ZNF226, ZNF227, ZNF229, ZNF23, ZNF230, ZNF232, ZNF233, ZNF234, ZNF235, ZNF236, ZNF239, ZNF24, ZNF248, ZNF25, ZNF250, ZNF251, ZNF253, ZNF254, ZNF256, ZNF257, ZNF26, ZNF260, ZNF263, ZNF264, ZNF266, ZNF267, ZNF268, ZNF273, ZNF274, ZNF275, ZNF276, ZNF277, ZNF28, ZNF280A, ZNF280B, ZNF280C, ZNF280D, ZNF281, ZNF282, ZNF283, ZNF284, ZNF285, ZNF286A, ZNF286B, ZNF287, ZNF292, ZNF296, ZNF3, ZNF30, ZNF300, ZNF302, ZNF304, ZNF311, ZNF316, ZNF317, ZNF318, ZNF319, ZNF32, ZNF320, ZNF322, ZNF324, ZNF324B, ZNF326, ZNF329, ZNF331, ZNF333, ZNF334, ZNF335, ZNF337, ZNF33A, ZNF33B, ZNF34, ZNF341, ZNF343, ZNF345, ZNF346, ZNF347, ZNF35, ZNF350, ZNF354A, ZNF354B, ZNF354C, ZNF358, ZNF362, ZNF365, ZNF366, ZNF367, ZNF37A, ZNF382, ZNF383, ZNF384, ZNF385A, ZNF385B, ZNF385C, ZNF385D, ZNF391, ZNF394, ZNF395, ZNF396, ZNF397, ZNF398, ZNF404, ZNF407, ZNF408, ZNF41, ZNF410, ZNF414, ZNF415, ZNF416, ZNF417, ZNF418, ZNF419, ZNF420, ZNF423, ZNF425, ZNF426, ZNF428, ZNF429, ZNF43, ZNF430, ZNF431, ZNF432, ZNF433, ZNF436, ZNF438, ZNF439, ZNF44, ZNF440, ZNF441, ZNF442, ZNF443, ZNF444, ZNF445, ZNF446, ZNF449, ZNF45, ZNF451, ZNF454, ZNF460, ZNF461, ZNF462, ZNF467, ZNF468, ZNF469, ZNF470, ZNF471, ZNF473, ZNF474, ZNF479, ZNF48, ZNF480, ZNF483, ZNF484, ZNF485, ZNF486, ZNF487, ZNF488, ZNF490, ZNF491, ZNF492, ZNF493, ZNF496, ZNF497, ZNF500, ZNF501, ZNF502, ZNF503, ZNF506, ZNF507, ZNF510, ZNF511, ZNF512, ZNF512B, ZNF513, ZNF514, ZNF516, ZNF517, ZNF518A, ZNF518B, ZNF519, ZNF521, ZNF524, ZNF525, ZNF526, ZNF527, ZNF528, ZNF529, ZNF530, ZNF532, ZNF534, ZNF536, ZNF540, ZNF541, ZNF543, ZNF544, ZNF546, ZNF547, NF548, ZNF549, ZNF550, ZNF551, ZNF552, ZNF554, ZNF555, ZNF556, ZNF557, ZNF558, ZNF559, ZNF560, ZNF561, ZNF562, ZNF563, ZNF564, ZNF565, ZNF566, ZNF567, ZNF568, ZNF569, ZNF57, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF623, ZNF624, ZNF625, ZNF626, ZNF627, ZNF628, ZNF629, ZNF630, ZNF639, ZNF641, ZNF644, ZNF645, ZNF646, ZNF648, ZNF649, ZNF652, ZNF653, ZNF654, ZNF655, ZNF658, ZNF66, ZNF660, ZNF662, ZNF664, ZNF665, ZNF667, ZNF668, ZNF669, ZNF670, ZNF671, ZNF672, ZNF674, ZNF675, ZNF676, ZNF677, ZNF678, ZNF679, ZNF680, ZNF681, ZNF682, ZNF683, ZNF684, ZNF687, ZNF688, ZNF689, ZNF69, ZNF691, ZNF692, ZNF695, ZNF696, ZNF697, ZNF699, ZNF7, ZNF70, ZNF700, ZNF701, ZNF703, ZNF704, ZNF705A, ZNF705B, ZNF705D, ZNF705E, ZNF705G, ZNF706, ZNF707, ZNF708, ZNF709, ZNF71, ZNF710, ZNF711, ZNF713, ZNF714, ZNF716, ZNF717, ZNF718, ZNF721, ZNF724, ZNF726, ZNF727, ZNF728, ZNF729, ZNF730, ZNF732, ZNF735, ZNF736, ZNF737, ZNF74, ZNF740, ZNF746, ZNF747, ZNF749, ZNF750, ZNF75A, ZNF75D, ZNF76, ZNF761, ZNF763, ZNF764, ZNF765, ZNF766, ZNF768, ZNF77, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788, ZNF789, ZNF79, ZNF790, ZNF791, ZNF792, ZNF793, ZNF799, ZNF8, ZNF80, ZNF800, ZNF804A, ZNF804B, ZNF805, ZNF808, ZNF81, ZNF813, ZNF814, ZNF816, ZNF821, ZNF823, ZNF827, ZNF829, ZNF83, ZNF830, ZNF831, ZNF835, ZNF836, ZNF837, ZNF84, ZNF841, ZNF843, ZNF844, ZNF845, ZNF846, ZNF85, ZNF850, ZNF852, ZNF853, ZNF860, ZNF865, ZNF878, ZNF879, ZNF880, ZNF883, ZNF888, ZNF891, ZNF90, ZNF91, ZNF92, ZNF93, ZNF98, ZNF99, ZSCAN1, ZSCAN10, ZSCAN12, ZSCAN16, ZSCAN18, ZSCAN2, ZSCAN20, ZSCAN21, ZSCAN22, ZSCAN23, ZSCAN25, ZSCAN26, ZSCAN29, ZSCAN30, ZSCAN31, ZSCAN32, ZSCAN4, ZSCAN5A, ZSCAN5B, ZSCAN5C, ZSCAN9, ZUFSP, ZXDA, ZXDB, ZXDC, and ZZZ3.

In some embodiments, B is selected from the group consisting of Hsp90 inhibitors, kinase inhibitors (e.g., cyclin dependent kinase inhibitors), MDM2 inhibitors, compounds targeting bromodomain-containing proteins, BET inhibitors, compounds targeting FKBP, HDAC inhibitors, lysine methyltransferase inhibitors, angiogenesis inhibitors, immunosuppressive compounds, and compounds targeting the aryl hydrocarbon receptor. In some embodiments, B is a Hsp90 inhibitor. In some embodiments, B is a kinase inhibitor (e.g., a cyclin dependent kinase inhibitor). In some embodiments, B is a MDM2 inhibitor. In some embodiments, B is a compound targeting bromodomain-containing protein (e.g., BRD4). In some embodiments, B is a bromodomain inhibitor (e.g., BRD4). In some embodiments, B is a BET inhibitor. In some embodiments, B is a compound targeting FKBP. In some embodiments, B is a HDAC inhibitor. In some embodiments, B is a lysine methyltransferase inhibitor. In some embodiments, B is an angiogenesis inhibitor. In some embodiments, B is an immunosuppressive compound. In some embodiments, B is a compound targeting the aryl hydrocarbon receptor.

In some embodiments, B is selected from the group consisting of group consisting of angiogenesis inhibitors, HDAC inhibitors, heat shock protein 90 (HSP90) inhibitors, human lysine methyltransferase inhibitors, immunosuppressive compounds, kinase inhibitors (e.g., cyclin dependent kinase inhibitors), and phosphatase inhibitors, MDM2 inhibitors. In some embodiments, B targets acyl-protein thioesterase-1 and -2 (APT1 and APT2), androgen receptor (AR), aryl hydrocarbon receptor (AHR), BET Bromodomain-containing proteins, estrogen receptor (ER), FKBP, HIV protease, HIV integrase, HCV protease, REF receptor kinase, or thyroid hormone receptor.

In some embodiments, B binds is a group that binds ABL, Akt, AMPK, and Era, Apaf-1, AR, A-Raf, ASK1, Ataxin-1, Aurora A, Aurora B, Aurora C, BAD, Bax, BCL-2, Bcl-xL, beta-catenin/TCF, BMI1, B-Raf, BRD2, BRD3, BRD4, BRK, BRM, BRSK I, BRSK2, BTK, C 1 delta, C 2, C₃G, CAMKK alpha, CAMKK beta, CAMKKI, CAS, Caspase-3, Caspase-6, Caspase-7, Caspase-9, catenin, Cbl, cdc25, cdc25A, CDC37, CDG4/6, CDK2, CDK9, c-Fos, CHK1/2, CK1 gamma, Clip, CLK2, C-Raf, CRK, CSK, C-TAK1, CXCR4, Cyclin DI, cyclin E, Cycline D, Cyto c, DAPK1, DDR2, DP-1, DYRK1A/2/3, E2F, EF2K, EGFR, EIF2A 3, ELK, EPH-A2/A4/B1/B2/B3/B4, ER81, ErbB3, Erk2, Erk5, Erk8, FADD, FAK, FAK, FGF-R1, FLIP, FoxOl, Fyn, Gap-1, GCK, GRB2, GSK-3, GSK3 beta, HDAC, HECTH9, HER4, HIPK 1/2/3/, HMGN1, HPK1, HRas, HSP70, HSP90, IGF-IR, IKK, IKK-alpha, IKK-beta, IKK-epsilon, IKK-gamma, IMP, IRA 2, IRAK4, IRAKI, IRE1, IRR, JAK, JNK1/2/3, KRas, Lamln A, LAMTOR2, Lck, LKB1, Lyn, MAP4K3, MAP4K5, MAPKAPK, MAPKAP-K2 K3, MAPKIb, MARK 1/2/3/4, MCAK, Mcl1, MDM2, ME K4, MEK1, MEK2, MEKKI, MEL, MINK1, MKK 1/2/3/4/6/7, MKK3, MKK6, MKP-3, MLK1/3, MN 1/2, MOS, MPK, MPSK1, MSK1, MST2/3/4, mTOR, mTORCI, Mucl, myc, Myc, Myc, NE 2a/6/7, NF-kappaB, NRas, NUAK1, OSR1, p19INK4D, p21 Cipl, p21 Wafl, p27 KIP1, p38 alpha/beta/delta/gamma MAPK, p53, p65RelA, p90RSK, PAK 1/2/4/5/6, PAX, PDGFRA, PDK1, PEA-15, PHK, pi 8 INK4, PI3K, PIM 1/2/3, PKA, PKB alpha/beta, PKC, PKC alpha/gamma/zeta, PKD, pl6 INK4A, PLC, PLDL Erkl, PLK1, PRAK, PRK2, PTEN, PYK2, RIP1, Rac, RACK-1, Rapl, Raptor, RAS, Rb, RIPK2, RIPK2, ROCK2, RSK1/2, SAP, SCF, SCF, SGK 1, SHC, SIK2/3, Smac, Smad2, Smad3, Smad4, Smad7, SmMLCK, SOS, spred, Spry, Src, Src, SRF, SRPK1, StaO, CREB, Statl, STK33, Suv39HI, Syk, TAB, TAK1, TAK1, Tal, TAO1, TBK1, TESK1, TGFBR1, TIE2, TIF la, TLK1, Tpl2, Tpl2/cotl, TRADD, TRAF2, TRAF3, TRAF6, TrkA, TSC2, TSSK1, TTBK1/2, TTK, UBF, ULK1/2, VEGFR1, WAVE-2, WNK 1, Wnt, XIAP, YES1, or ZAP70.

In some embodiments, B is any ligand (e.g., a ligand of a protein of interest) disclosed in: Li, X., Song, Y. Proteolysis-targeting chimera (PROTAC) for targeted protein degradation and cancer therapy. J Hematol Oncol 13, 50 (2020); Scheepstra, M., Hekking, K., van Hijfte, L., & Folmer, R. Bivalent Ligands for Protein Degradation in Drug Discovery. Computational and structural biotechnology journal, 17, 160-176 (2019).

In some embodiments, B is a ligand as disclosed in PCT publication WO/2019/165216, WO/2016/105518, WO/2019/165229, WO/2017/007612, or WO/2020/006157; or US publication 2021/0015929 or 2019/0175572, each of which is incorporated herein by reference.

In some embodiments, B is selected from the group consisting of JQ1, I-BET-762, OTX-015, I-BET-151, TEN-010, CPI-203, PFI-1, MS436, RVX-297, RVX-208, ABBV-744, CPI-0610, HJB97, rapamycin, FK506, GPI1046, GPI1485, V10367, ElteN378, everolimus, tacrolimus, ridaforolimus, zotarolimus, 3BDO, iRap, AP2167, cRap, pRap, AP23102, AP1510, AP1903, Shield-1, AP20187, ibrutinib, N-piperidine ibrutinib, quizartinib, BI-4464, molibresib, abemaciclib, N-deshydroxyethyl dasatinib, SI-109, navitoclax-piperazine, androstanolone acetate, palbociclib-propargyl, SMARCA-BD, and SLF.

In some embodiments, B is selected from the group consisting of JQ1, I-BET-762, OTX-015, I-BET-151, TEN-010, CPI-203, PFI-1, MS436, RVX-297, RVX-208, ABBV-744, CPI-0610, HJB97, rapamycin, FK506, GPI1046, GPI1485, V10367, ElteN378, everolimus, tacrolimus, ridaforolimus, zotarolimus, 3BDO, iRap, AP2167, cRap, pRap, AP23102, AP1510, AP1903, Shield-1, AP20187, ibrutinib, N-piperidine ibrutinib, quizartinib, BI-4464, molibresib, abemaciclib, N-deshydroxyethyl dasatinib, dasatinib, SI-109, navitoclax, navitoclax-piperazine, androstanolone acetate, palbociclib, palbociclib-propargyl, SMARCA-BD, and SLF.

In some embodiments, B is selected from the group consisting of JQ1, I-BET-762, OTX-015, I-BET-151, TEN-010, CPI-203, PFI-1, MS436, RVX-297, RVX-208, ABBV-744, CPI-0610, and HJB97. In certain embodiments, B is selected from the group consisting of:

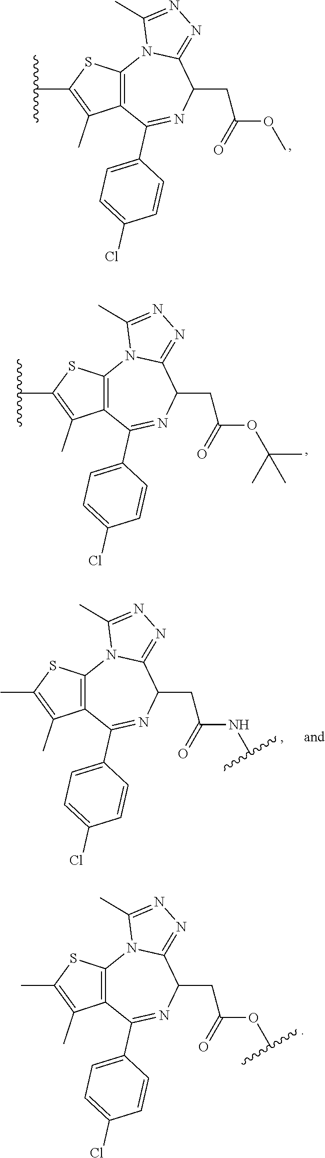

In some embodiments, B is a FKBP binder. In some embodiments, the FKBP binder is a FKBP12 binder. In some embodiments, B is:

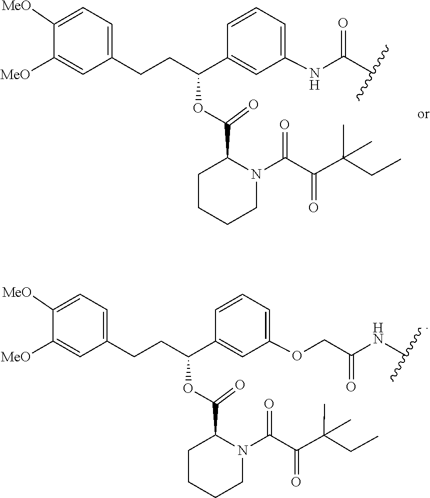 [SMILES: *C(=O)Nc1cccc([C@@H](CCc2ccc(OC)c(OC)c2)OC(=O)[C@@H]2CCCCN2C(=O)C(=O)C(C)(C)CC)c1 ; *NC(=O)COc1cccc([C@@H](CCc2ccc(OC)c(OC)c2)OC(=O)[C@@H]2CCCCN2C(=O)C(=O)C(C)(C)CC)c1]

In some embodiments, B is selected from the group consisting of rapamycin, FK506, GPI1046, GPI1485, V10367, ElteN378, everolimus, tacrolimus, ridaforolimus, zotarolimus, 3BDO, iRap, AP2167, cRap, pRap, AP23102, AP1510, AP1903, Shield-1, and AP20187.

In some embodiments, B is:

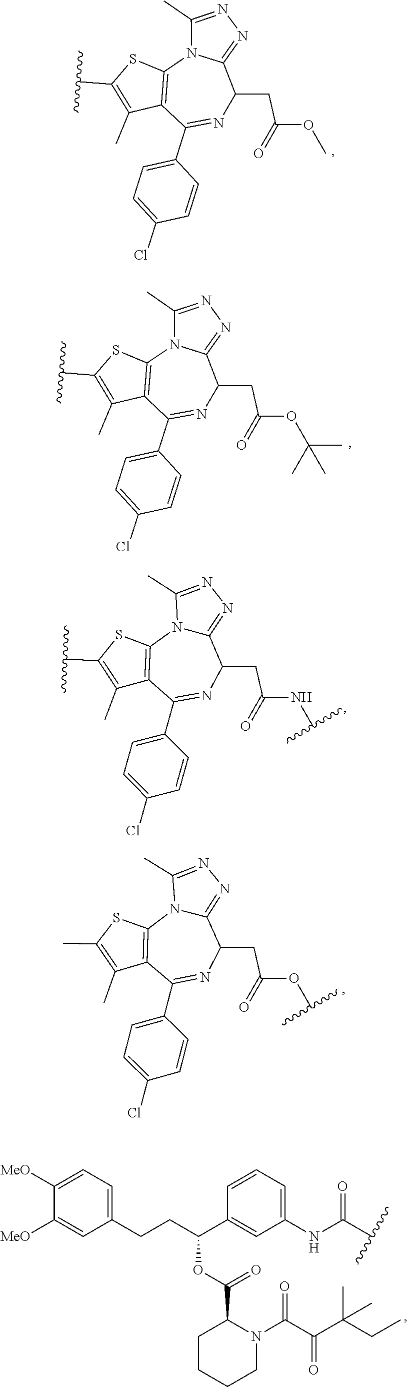

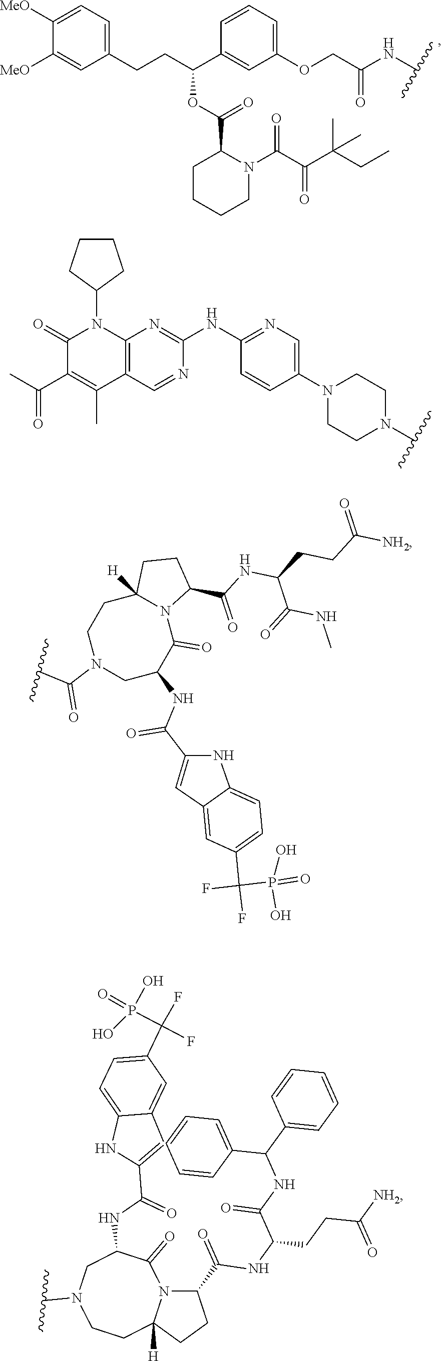 [SMILES: *NC(=O)COc1cccc([C@@H](CCc2ccc(OC)c(OC)c2)OC(=O)[C@@H]2CCCCN2C(=O)C(=O)C(C)(C)CC)c1 ; *N1CCN(c2ccc(Nc3ncc4c(C)c(C(C)=O)c(=O)n(C5CCCC5)c4n3)nc2)CC1 ; *C(=O)N1CC[C@@H]2CC[C@@H](C(=O)N[C@@H](CCC(N)=O)C(=O)NC)N2C(=O)[C@@H](NC(=O)c2cc3cc(C(F)(F)P(=O)(O)O)ccc3[nH]2)C1 ; *N1CC[C@H]2CC[C@@H](C(=O)N[C@@H](CCC(N)=O)C(=O)NC(c3ccccc3)c3ccccc3)N2C(=O)[C@@H](NC(=O)c2cc3cc(C(F)(F)P(=O)(O)O)ccc3[nH]2)C1]

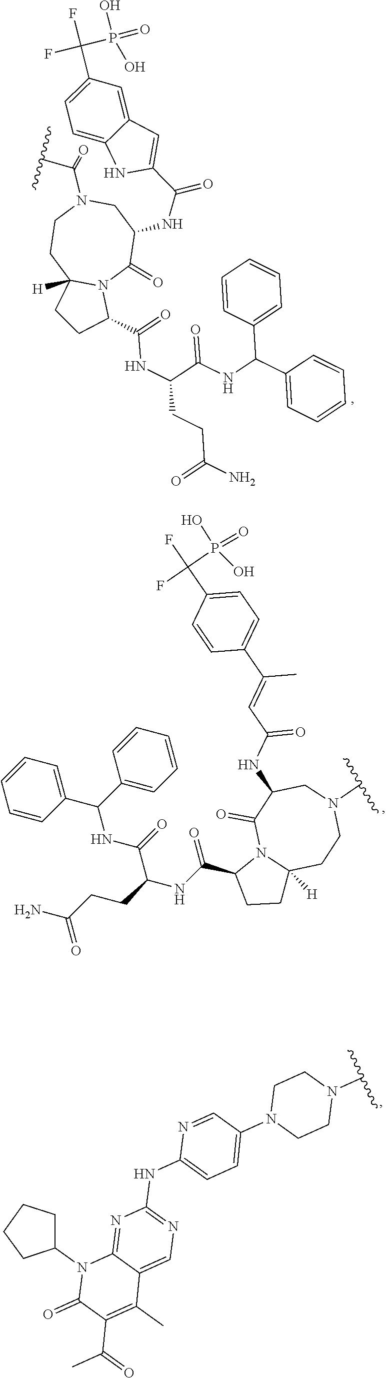

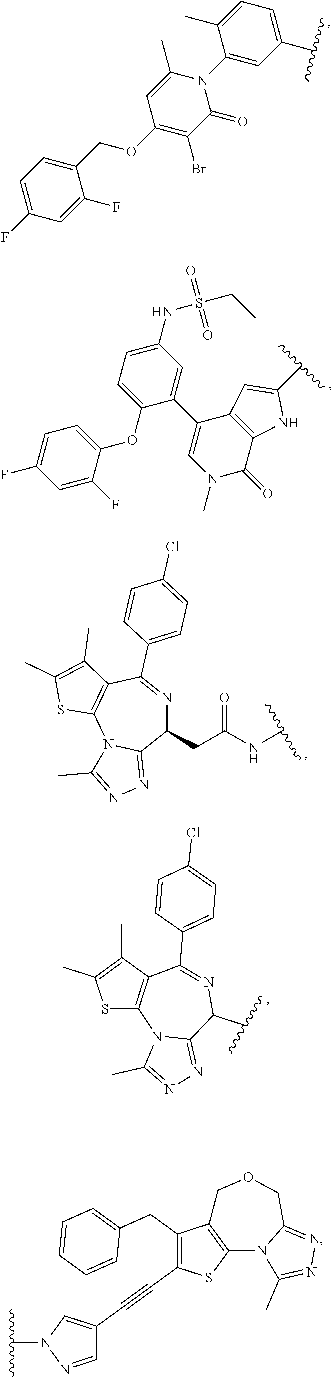 [SMILES: *c1ccc(C)c(-n2c(C)cc(OCc3ccc(F)cc3F)c(Br)c2=O)c1 ; *c1cc2c(-c3cc(NS(=O)(=O)CC)ccc3Oc3ccc(F)cc3F)cn(C)c(=O)c2[nH]1 ; *NC(=O)C[C@@H]1N=C(c2ccc(Cl)cc2)c2c(sc(C)c2C)-n2c(C)nnc21 ; *C1N=C(c2ccc(Cl)cc2)c2c(sc(C)c2C)-n2c(C)nnc21 ; *n1cc(C#Cc2sc3c(c2Cc2ccccc2)COCc2nnc(C)n2-3)cn1]

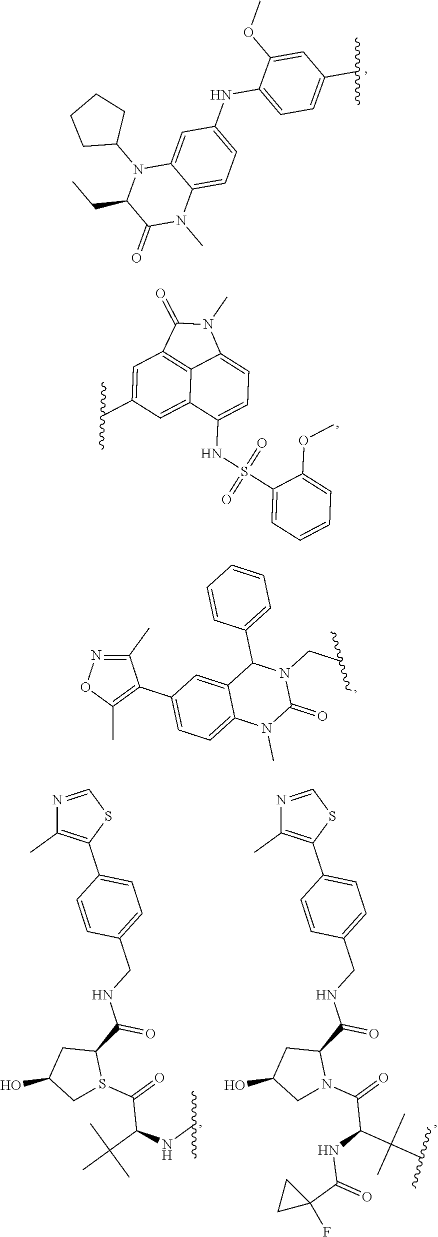

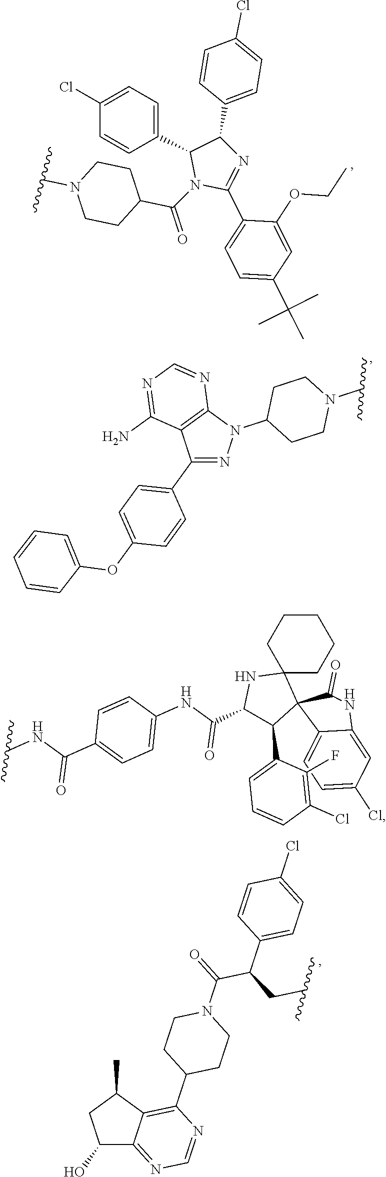

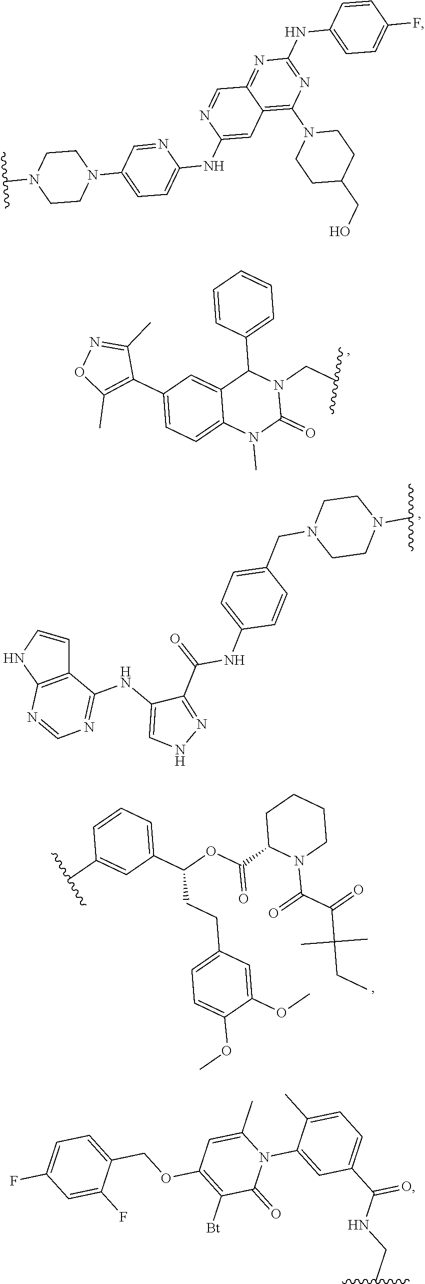

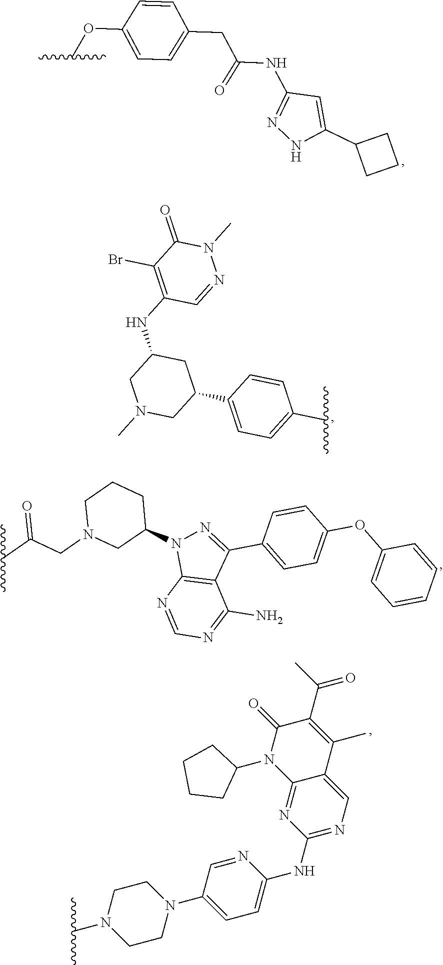 [SMILES: *Oc1ccc(CC(=O)Nc2cc(C3CCC3)[nH]n2)cc1 ; *c1ccc([C@H]2C[C@@H](Nc3cnn(C)c(=O)c3Br)CN(C)C2)cc1 ; *C(=O)CN1CCC[C@@H](n2nc(-c3ccc(Oc4ccccc4)cc3)c3c(N)ncnc32)C1 ; *N1CCN(c2ccc(Nc3ncc4c(C)c(C(C)=O)c(=O)n(C5CCCC5)c4n3)nc2)CC1]

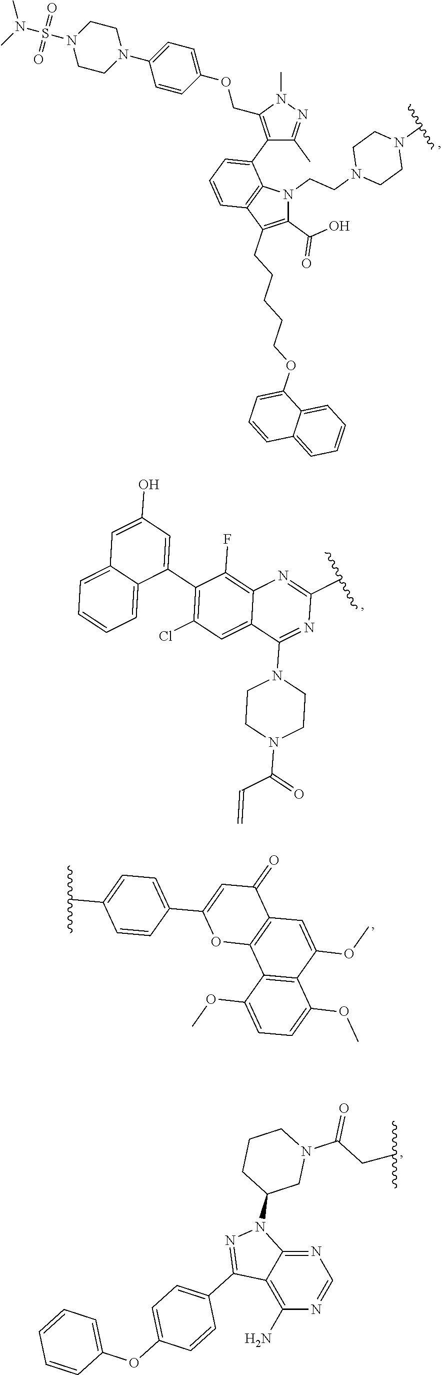 [SMILES: *N1CCN(CCn2c(C(=O)O)c(CCCCCOc3cccc4ccccc34)c3cccc(-c4c(C)nn(C)c4COc4ccc(N5CCN(S(=O)(=O)N(C)C)CC5)cc4)c32)CC1 ; *c1nc(N2CCN(C(=O)C=C)CC2)c2cc(Cl)c(-c3cc(O)cc4ccccc34)c(F)c2n1 ; *c1ccc(-c2cc(=O)c3cc(OC)c4c(OC)ccc(OC)c4c3o2)cc1 ; *CC(=O)N1CCC[C@H](n2nc(-c3ccc(Oc4ccccc4)cc3)c3c(N)ncnc32)C1]

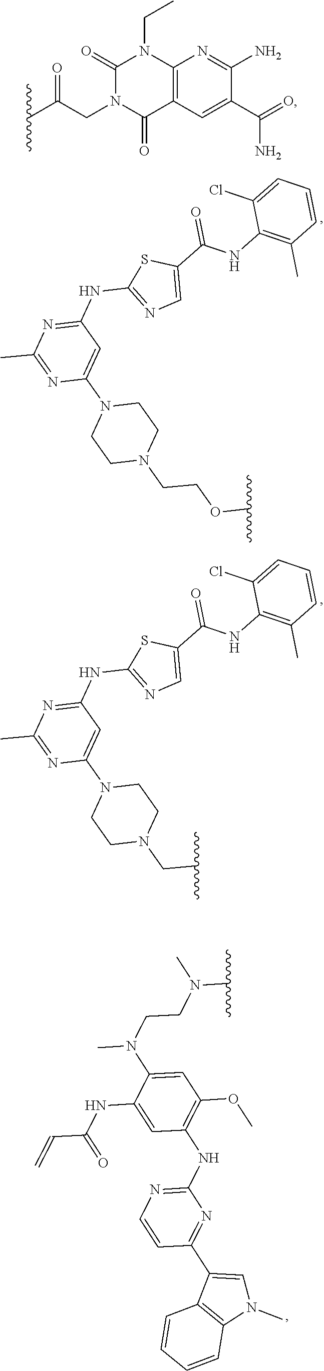

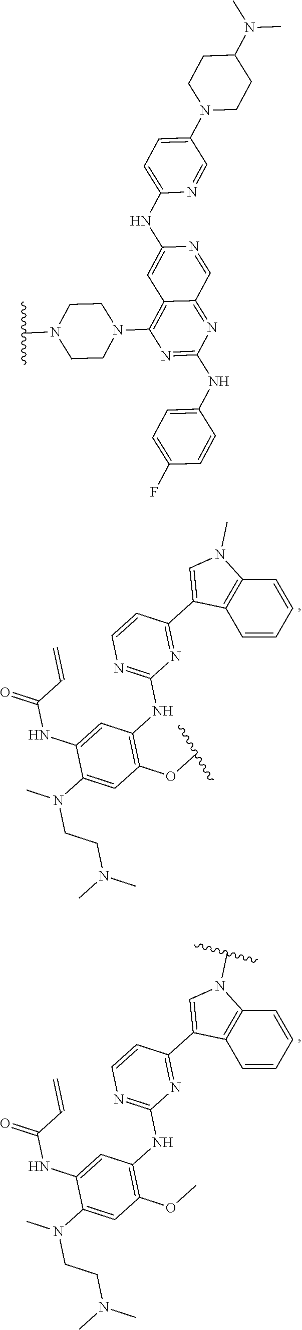 [SMILES: *N1CCN(c2nc(Nc3ccc(F)cc3)nc3cnc(Nc4ccc(N5CCC(N(C)C)CC5)cn4)cc23)CC1 ; *Oc1cc(N(C)CCN(C)C)c(NC(=O)C=C)cc1Nc1nccc(-c2cn(C)c3ccccc23)n1 ; *n1cc(-c2ccnc(Nc3cc(NC(=O)C=C)c(N(C)CCN(C)C)cc3OC)n2)c2ccccc21]

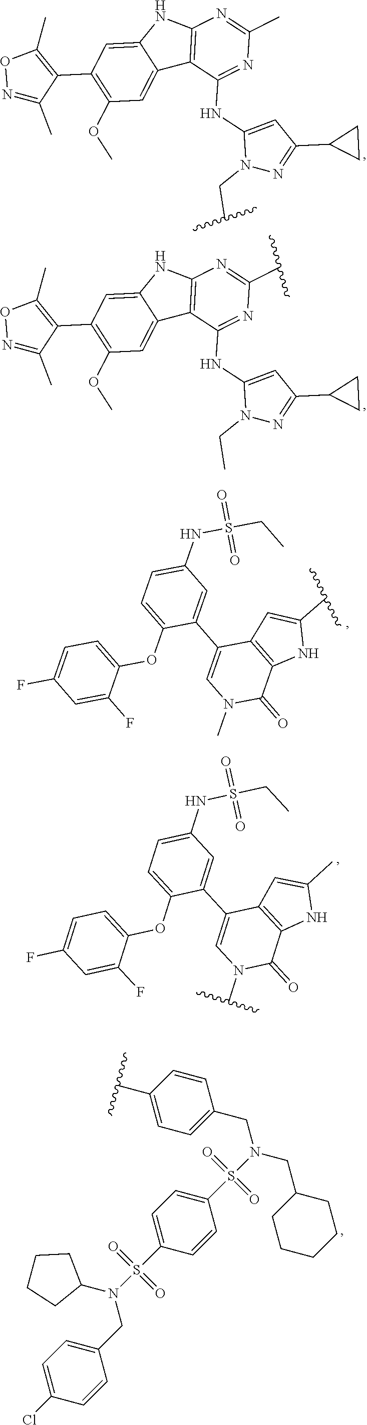

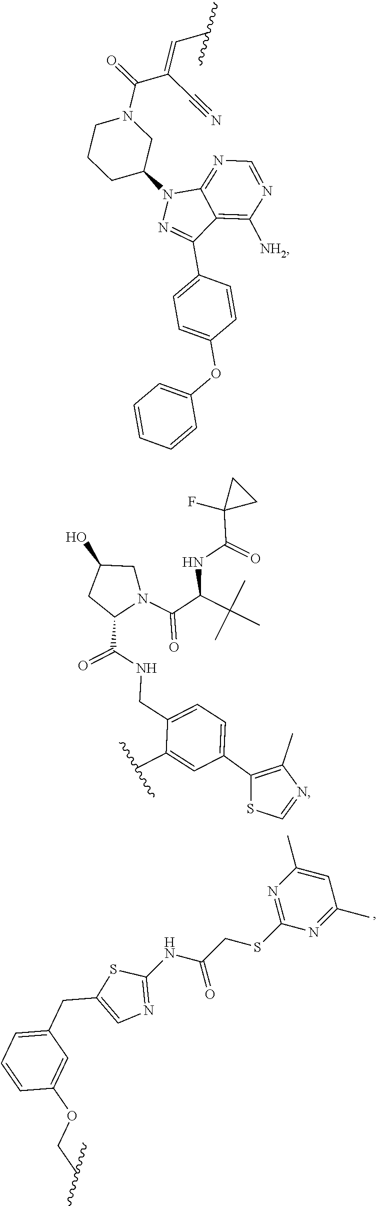

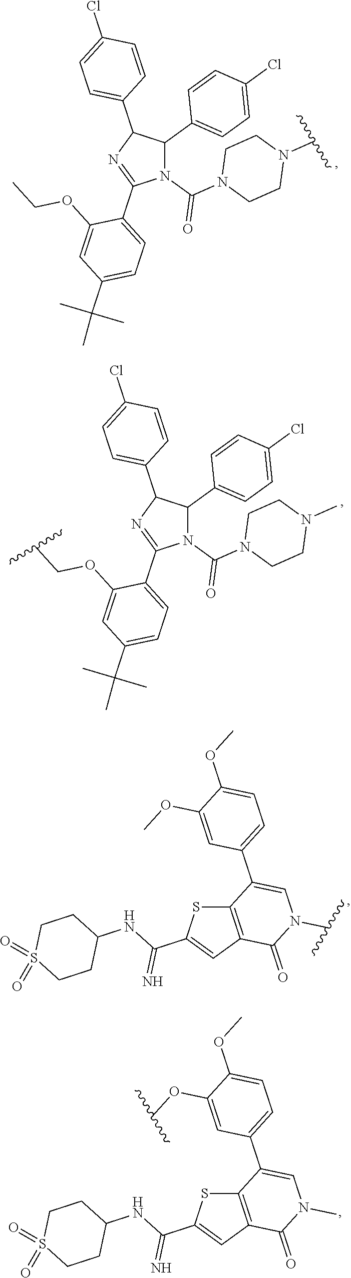

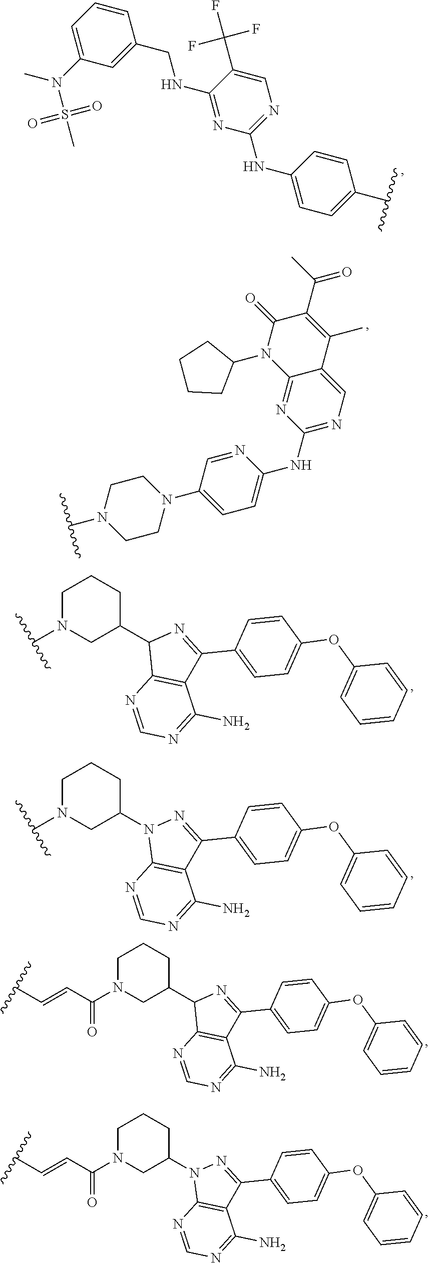

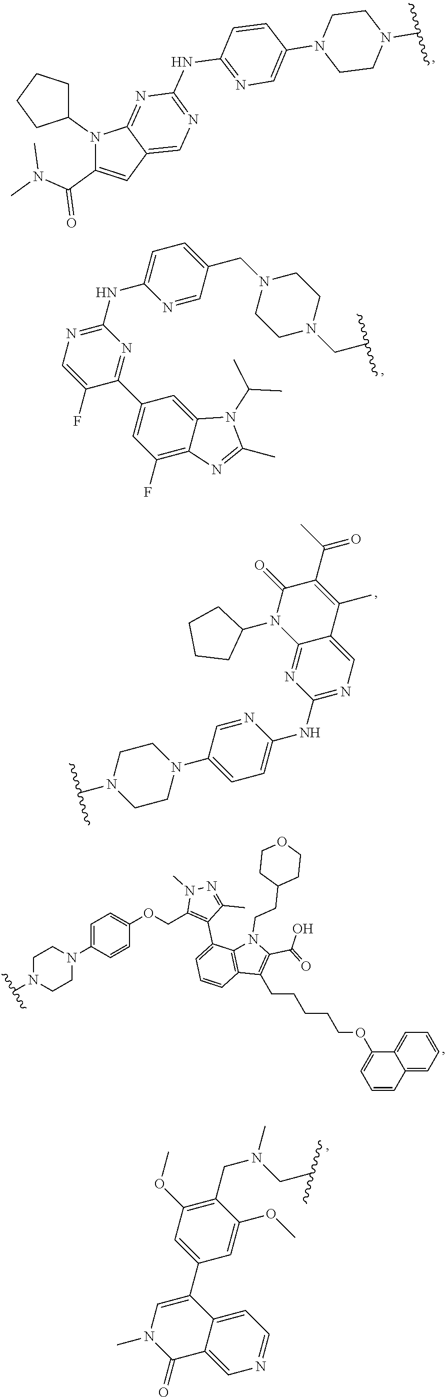

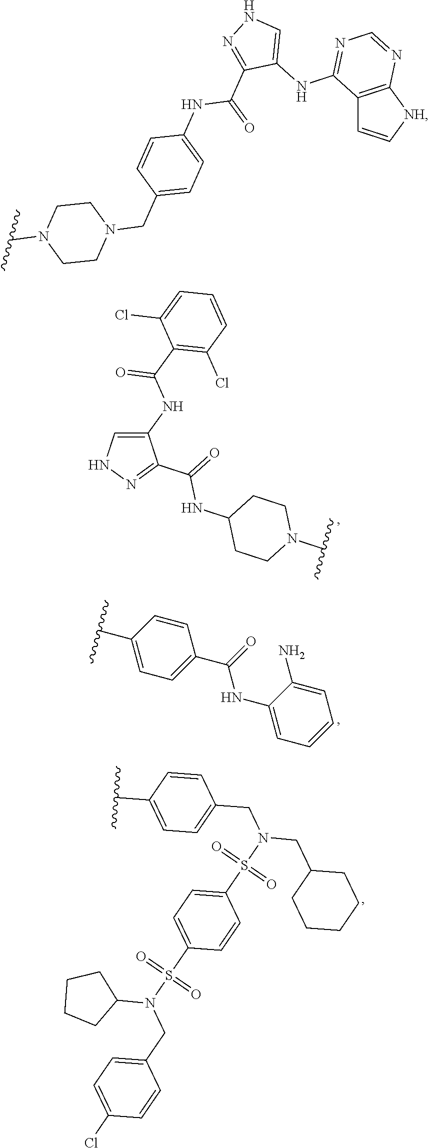 [SMILES: *N1CCN(Cc2ccc(NC(=O)c3n[nH]cc3Nc3ncnc4[nH]ccc34)cc2)CC1 ; *N1CCC(NC(=O)c2n[nH]cc2NC(=O)c2c(Cl)cccc2Cl)CC1 ; *c1ccc(C(=O)Nc2ccccc2N)cc1 ; *c1ccc(CN(CC2CCCCC2)S(=O)(=O)c2ccc(S(=O)(=O)N(Cc3ccc(Cl)cc3)C3CCCC3)cc2)cc1]

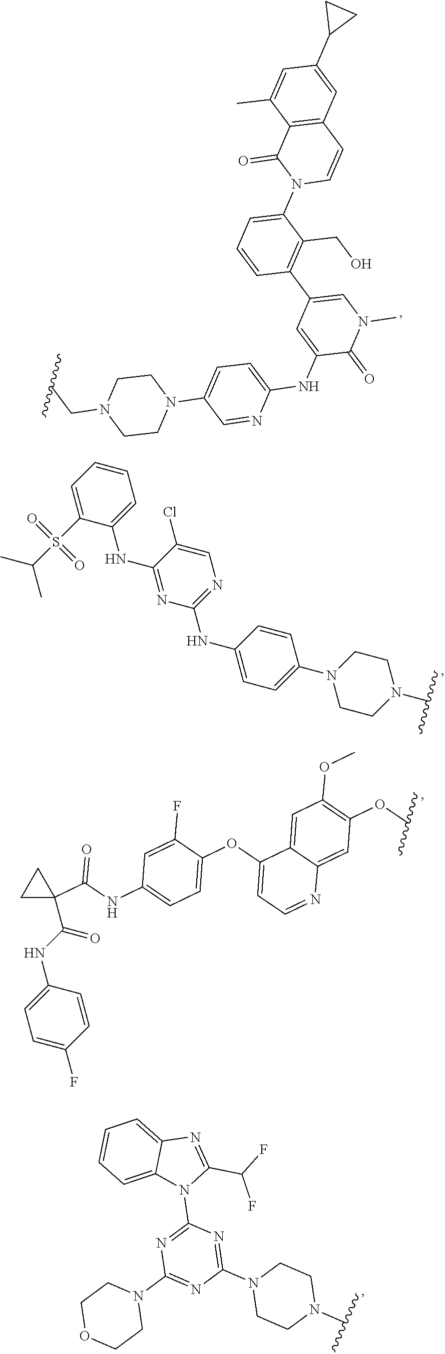

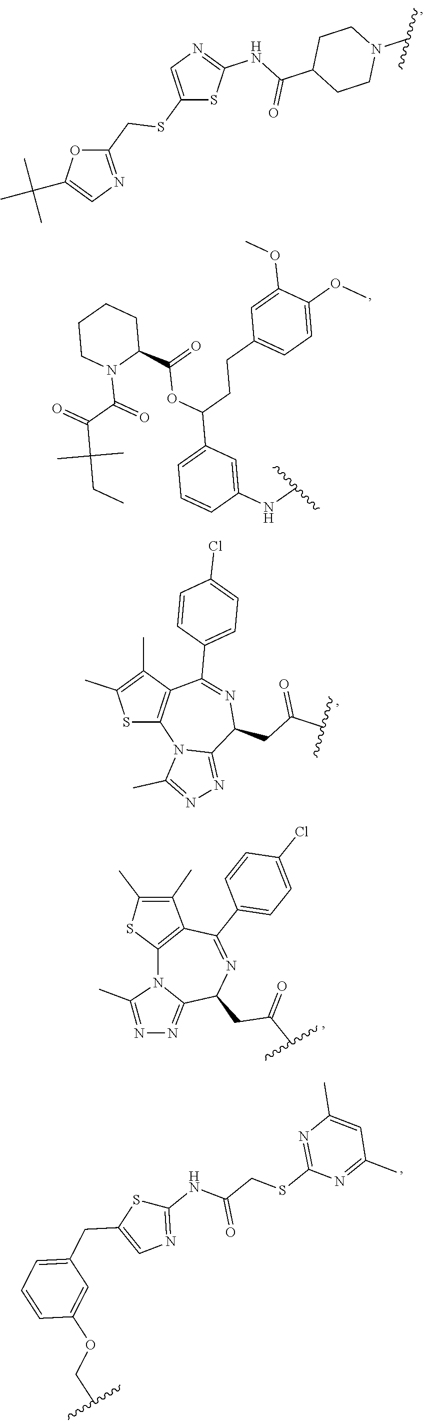

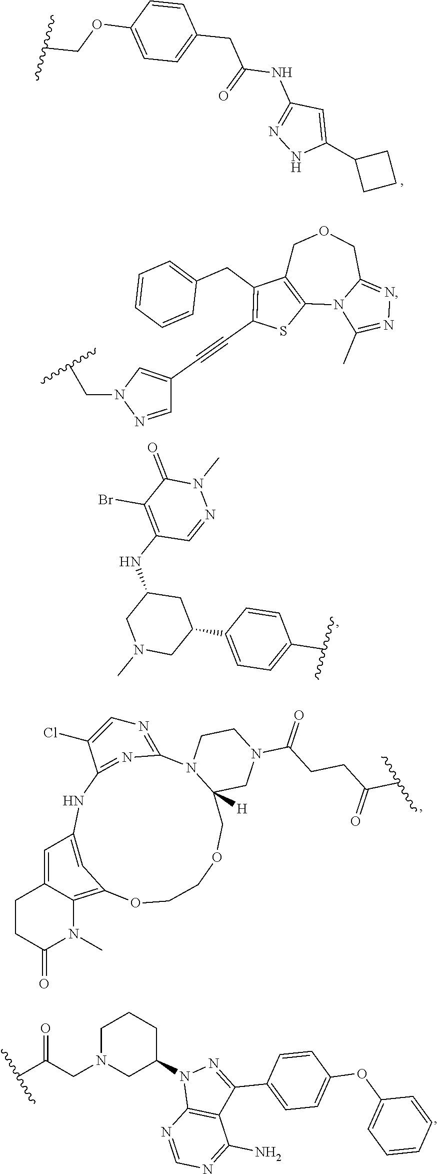

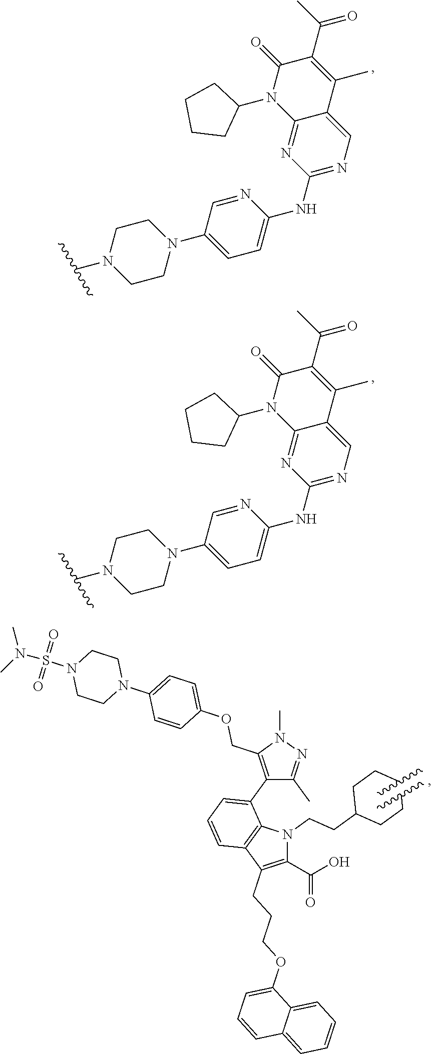

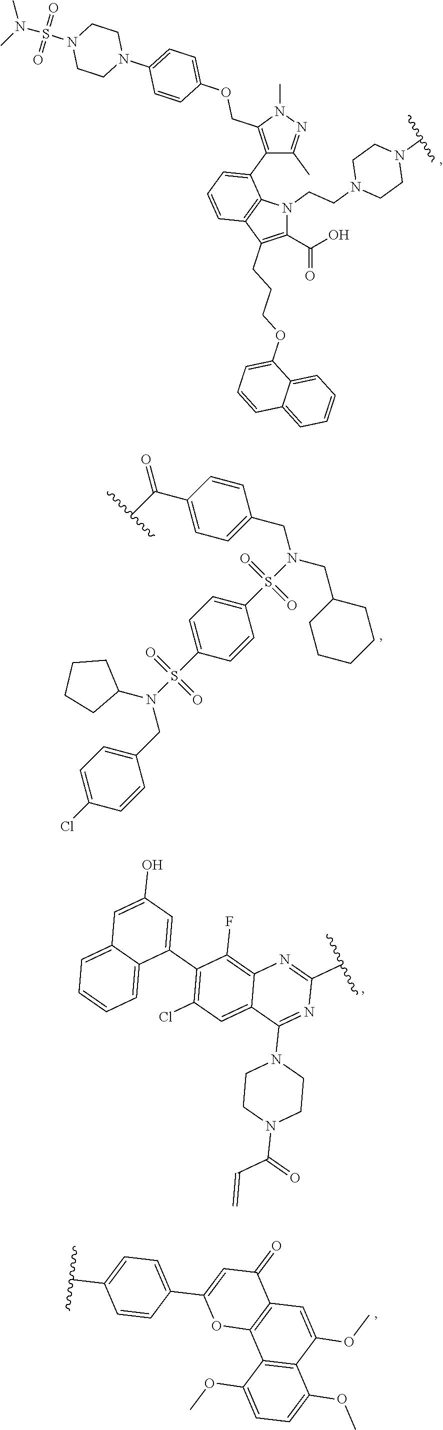 [SMILES: *N1CCN(CCn2c(C(=O)O)c(CCCOc3cccc4ccccc34)c3cccc(-c4c(C)nn(C)c4COc4ccc(N5CCN(S(=O)(=O)N(C)C)CC5)cc4)c32)CC1 ; *C(=O)c1ccc(CN(CC2CCCCC2)S(=O)(=O)c2ccc(S(=O)(=O)N(Cc3ccc(Cl)cc3)C3CCCC3)cc2)cc1 ; *c1nc(N2CCN(C(=O)C=C)CC2)c2cc(Cl)c(-c3cc(O)cc4ccccc34)c(F)c2n1 ; *c1ccc(-c2cc(=O)c3cc(OC)c4c(OC)ccc(OC)c4c3o2)cc1]

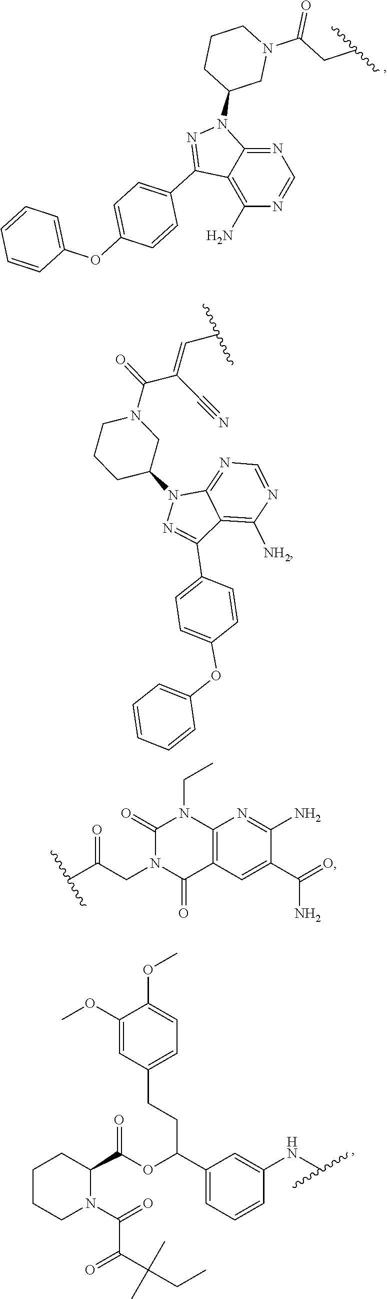

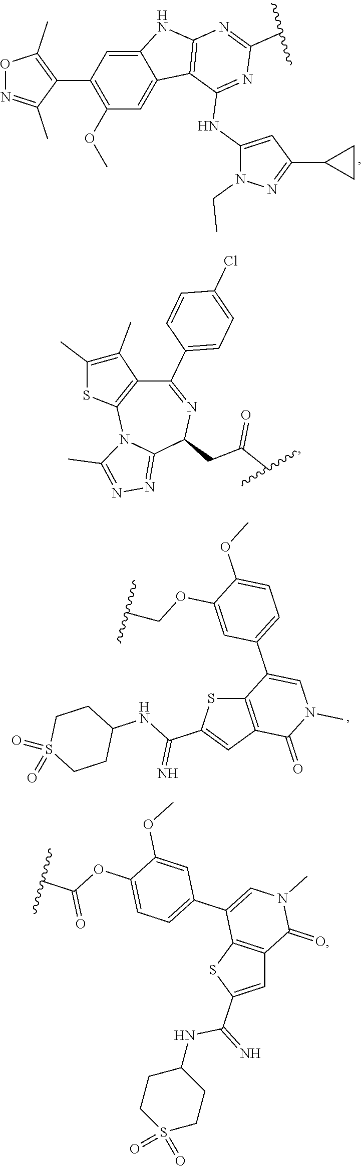

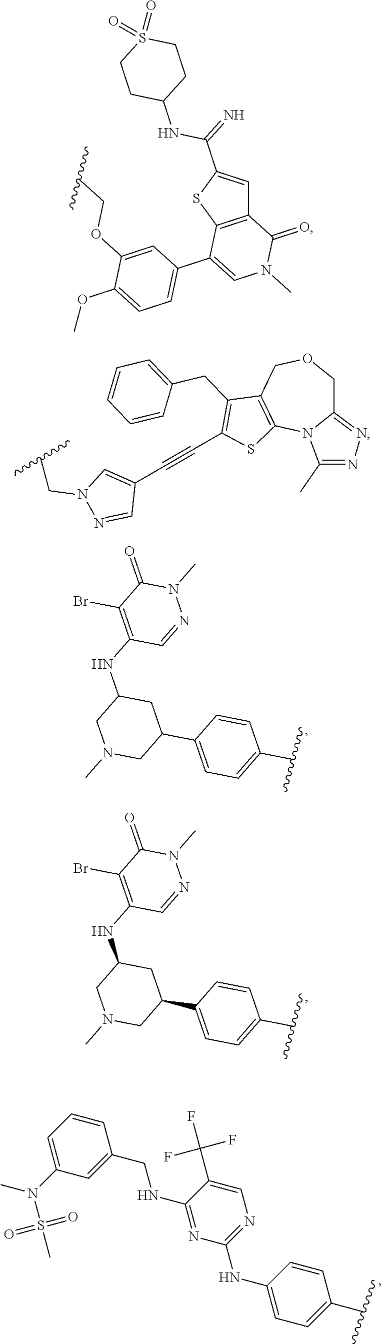

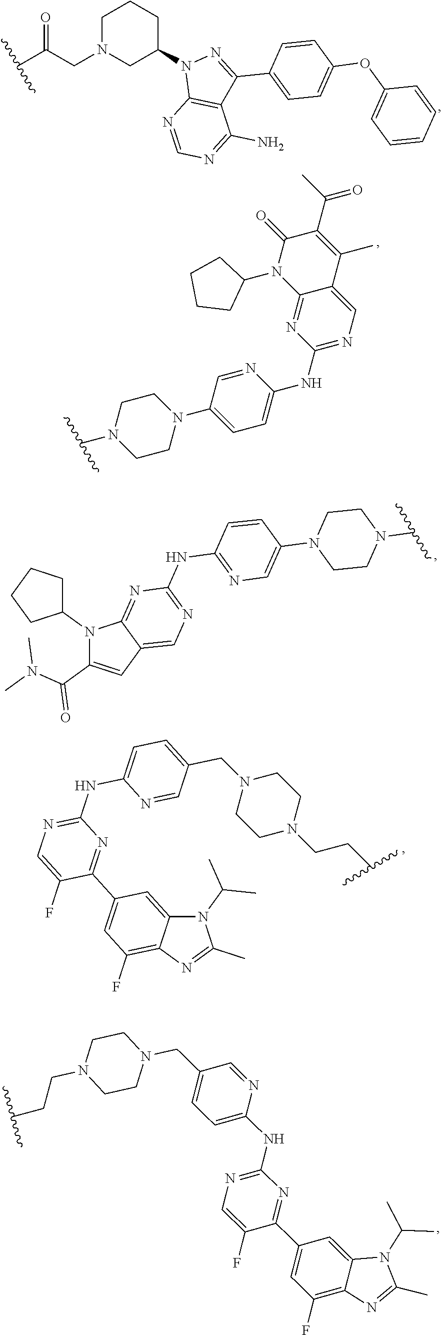

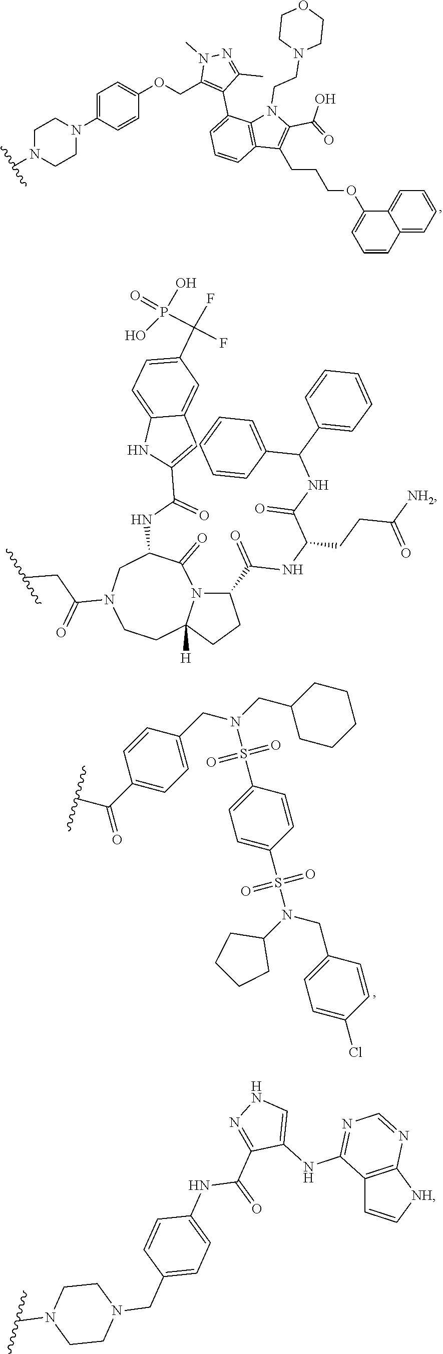 [SMILES: *N1CCN(c2ccc(OCc3c(-c4cccc5c(CCCOc6cccc7ccccc67)c(C(=O)O)n(CCN6CCOCC6)c45)c(C)nn3C)cc2)CC1 ; *CC(=O)N1CC[C@H]2CC[C@@H](C(=O)N[C@@H](CCC(N)=O)C(=O)NC(c3ccccc3)c3ccccc3)N2C(=O)[C@@H](NC(=O)c2cc3cc(C(F)(F)P(=O)(O)O)ccc3[nH]2)C1 ; *C(=O)c1ccc(CN(CC2CCCCC2)S(=O)(=O)c2ccc(S(=O)(=O)N(Cc3ccc(Cl)cc3)C3CCCC3)cc2)cc1 ; *N1CCN(Cc2ccc(NC(=O)c3n[nH]cc3Nc3ncnc4[nH]ccc34)cc2)CC1]

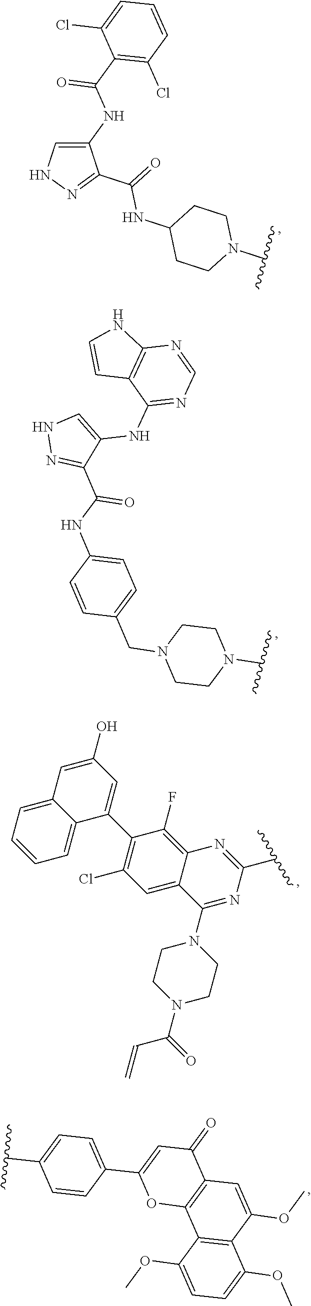 [SMILES: *N1CCC(NC(=O)c2n[nH]cc2NC(=O)c2c(Cl)cccc2Cl)CC1 ; *N1CCN(Cc2ccc(NC(=O)c3n[nH]cc3Nc3ncnc4[nH]ccc34)cc2)CC1 ; *c1nc(N2CCN(C(=O)C=C)CC2)c2cc(Cl)c(-c3cc(O)cc4ccccc34)c(F)c2n1 ; *c1ccc(-c2cc(=O)c3cc(OC)c4c(OC)ccc(OC)c4c3o2)cc1]

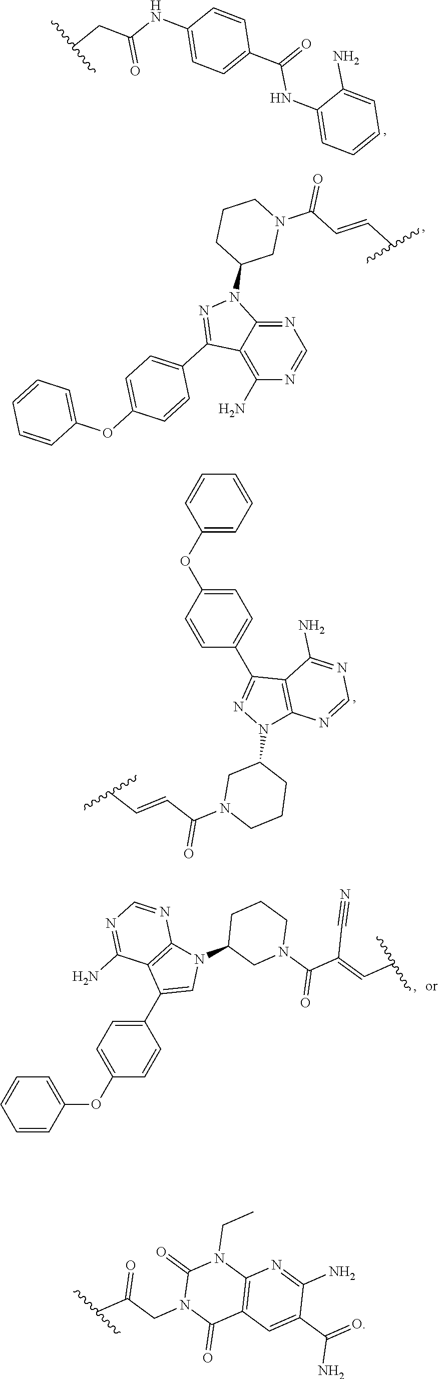

In some embodiments, B is

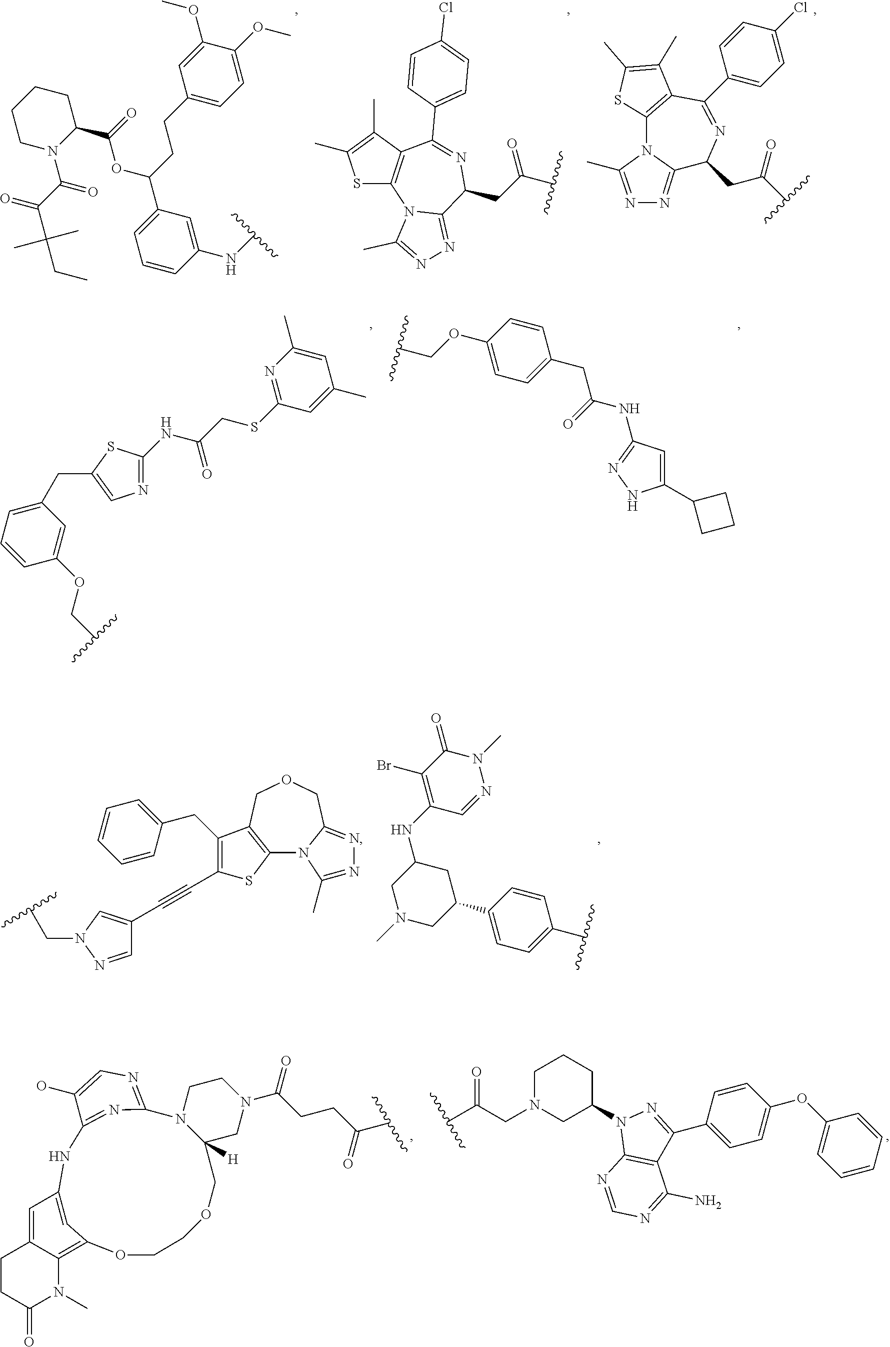

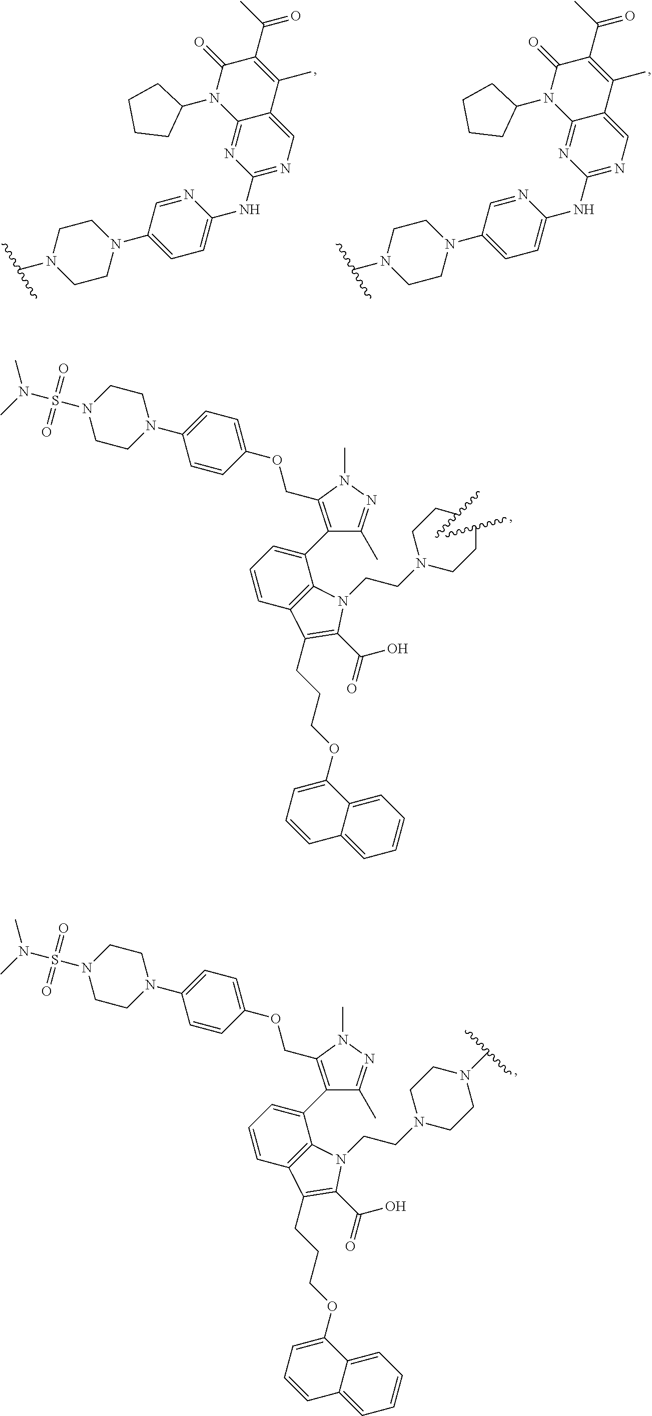 [SMILES: *N1CCN(c2ccc(Nc3ncc4c(C)c(C(C)=O)c(=O)n(C5CCCC5)c4n3)nc2)CC1 ; *N1CCN(c2ccc(Nc3ncc4c(C)c(C(C)=O)c(=O)n(C5CCCC5)c4n3)nc2)CC1 ; *CCN(CC*)CCn1c(C(=O)O)c(CCCOc2cccc3ccccc23)c2cccc(-c3c(C)nn(C)c3COc3ccc(N4CCN(S(=O)(=O)N(C)C)CC4)cc3)c21 ; *N1CCN(CCn2c(C(=O)O)c(CCCOc3cccc4ccccc34)c3cccc(-c4c(C)nn(C)c4COc4ccc(N5CCN(S(=O)(=O)N(C)C)CC5)cc4)c32)CC1]

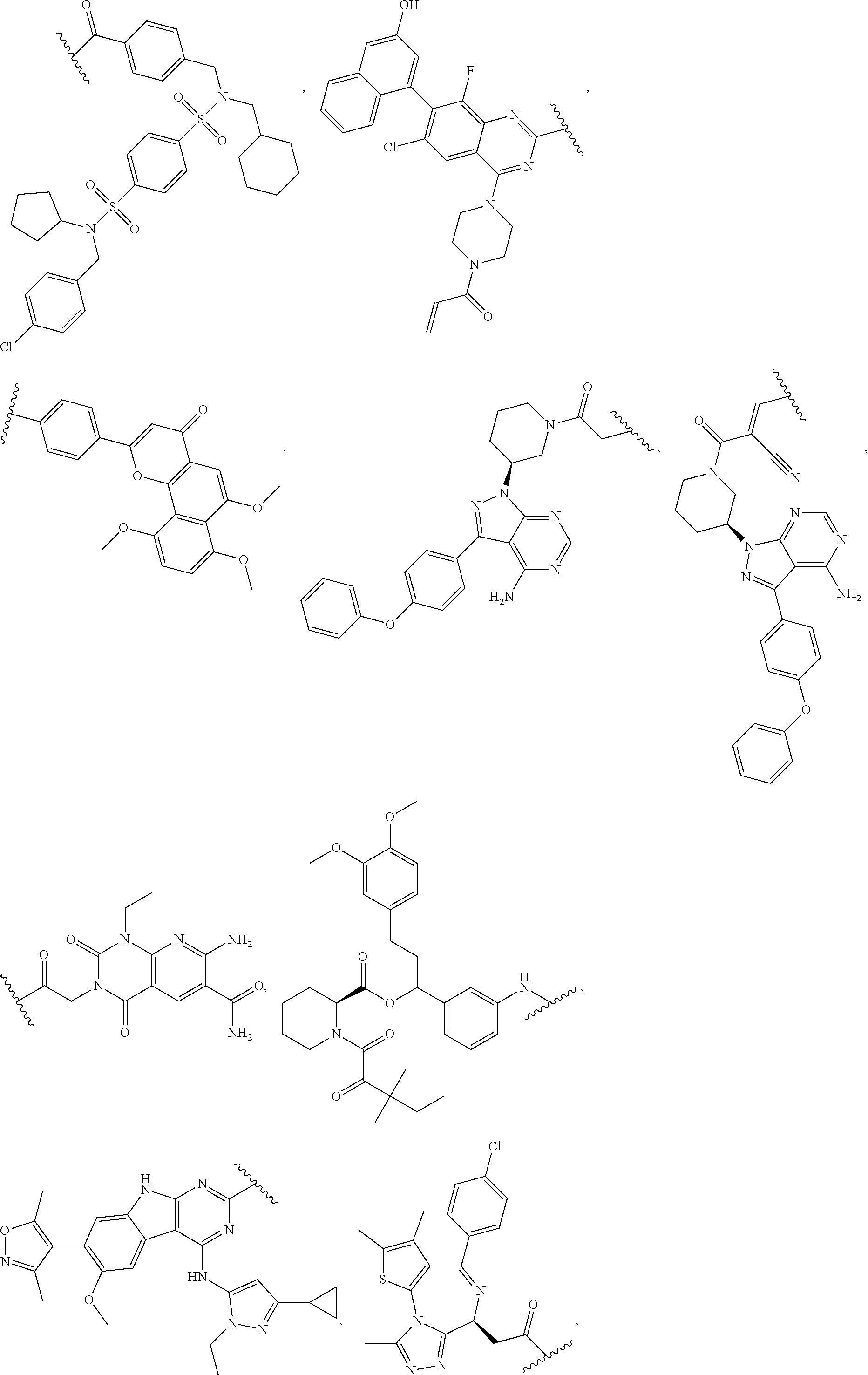

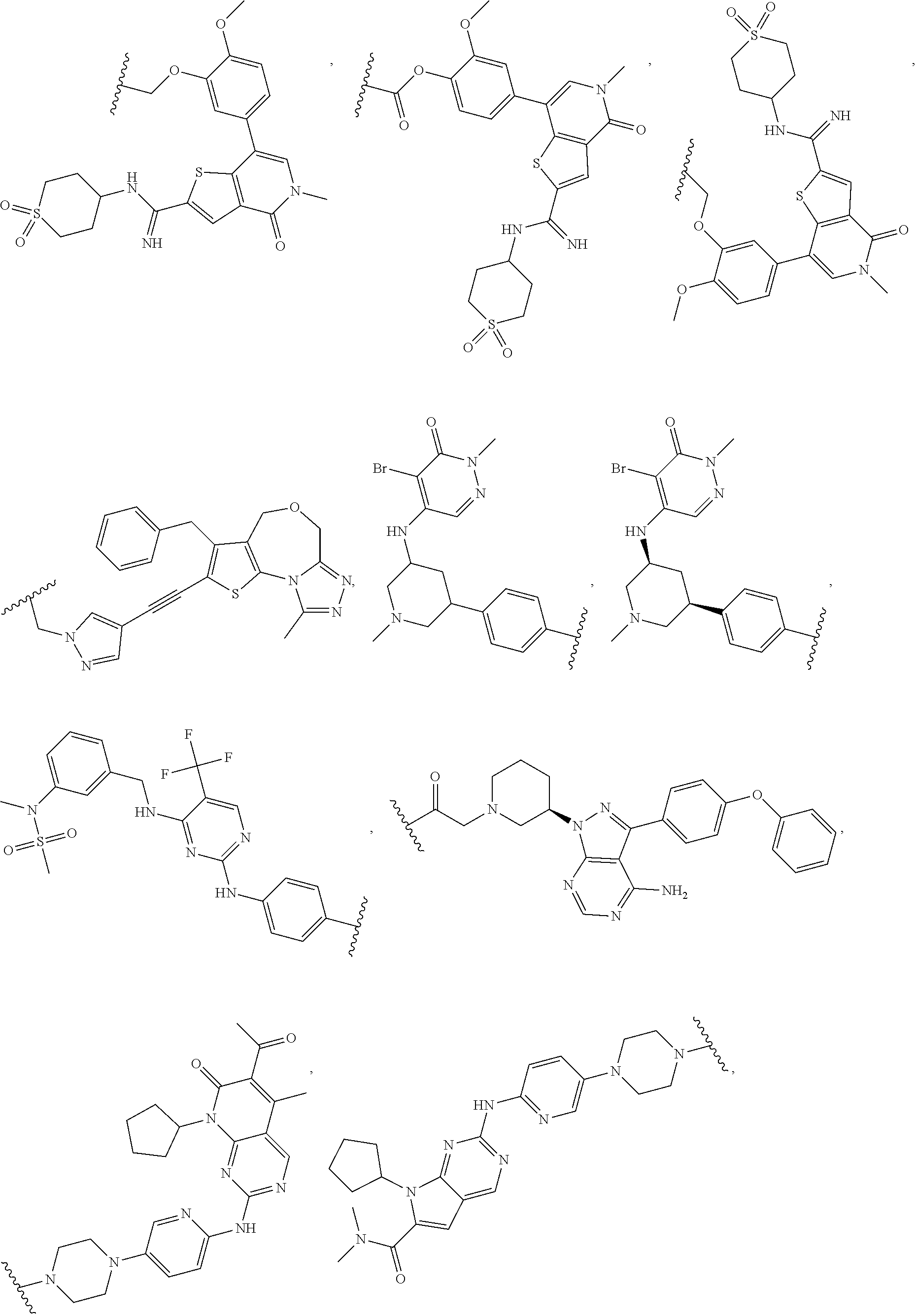

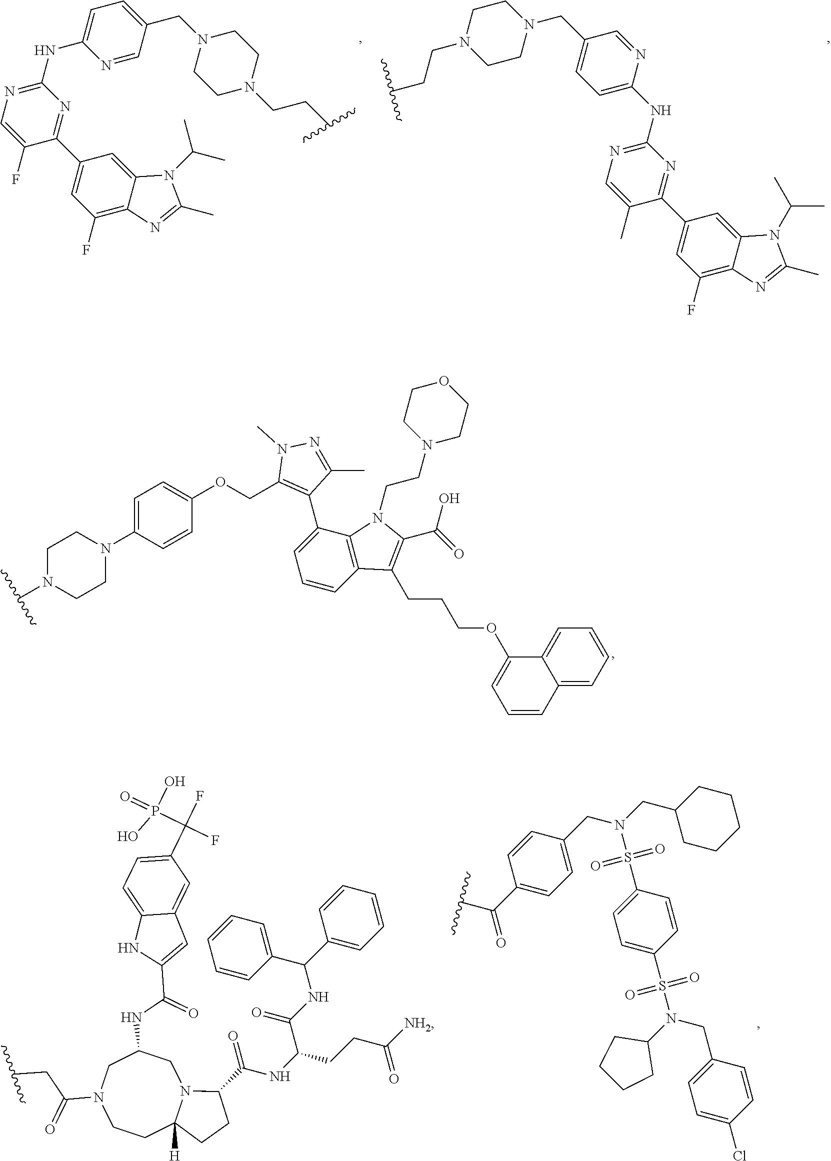 [SMILES: *CCN1CCN(Cc2ccc(Nc3ncc(F)c(-c4cc(F)c5nc(C)n(C(C)C)c5c4)n3)nc2)CC1 ; *CCN1CCN(Cc2ccc(Nc3ncc(C)c(-c4cc(F)c5nc(C)n(C(C)C)c5c4)n3)nc2)CC1 ; *N1CCN(c2ccc(OCc3c(-c4cccc5c(CCCOc6cccc7ccccc67)c(C(=O)O)n(CCN6CCOCC6)c45)c(C)nn3C)cc2)CC1 ; *CC(=O)N1CC[C@H]2CC[C@@H](C(=O)N[C@@H](CCC(N)=O)C(=O)NC(c3ccccc3)c3ccccc3)N2C[C@@H](NC(=O)c2cc3cc(C(F)(F)P(=O)(O)O)ccc3[nH]2)C1 ; *C(=O)c1ccc(CN(CC2CCCCC2)S(=O)(=O)c2ccc(S(=O)(=O)N(Cc3ccc(Cl)cc3)C3CCCC3)cc2)cc1]

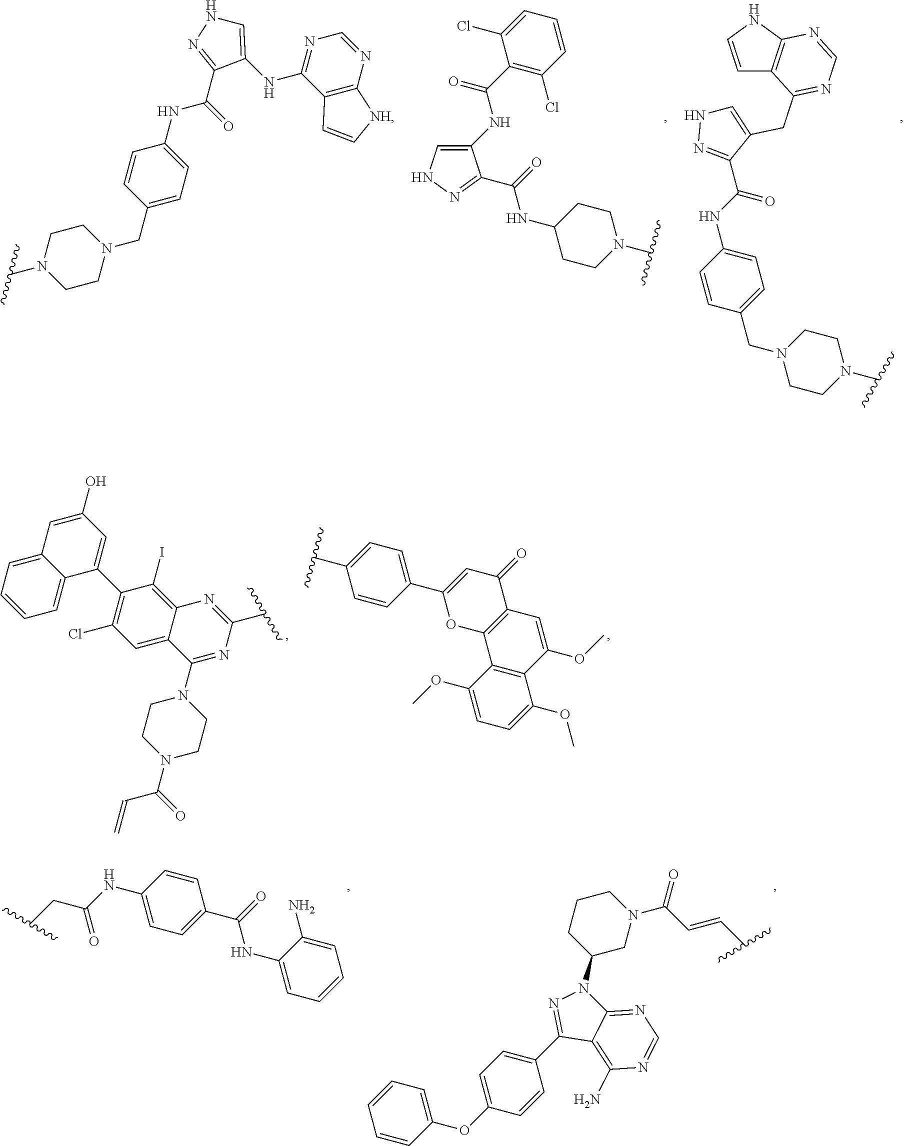

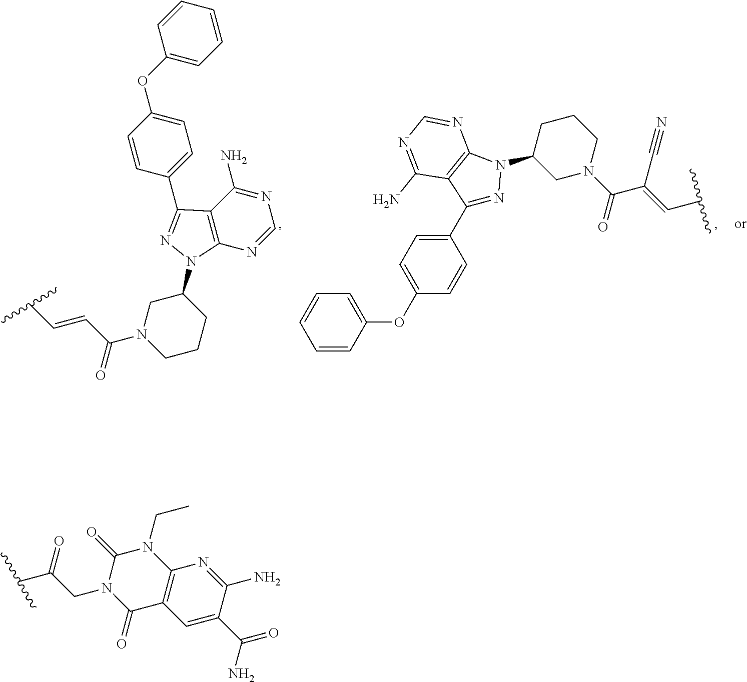 [SMILES: */C=C/C(=O)N1CCC[C@H](n2nc(-c3ccc(Oc4ccccc4)cc3)c3c(N)ncnc32)C1 ; */C=C(\C#N)C(=O)N1CCC[C@H](n2nc(-c3ccc(Oc4ccccc4)cc3)c3c(N)ncnc32)C1 ; *C(=O)Cn1c(=O)c2cc(C(N)=O)c(N)nc2n(CC)c1=O]

In some embodiments, the compound of Formula (I′) or (I) is of the formula:

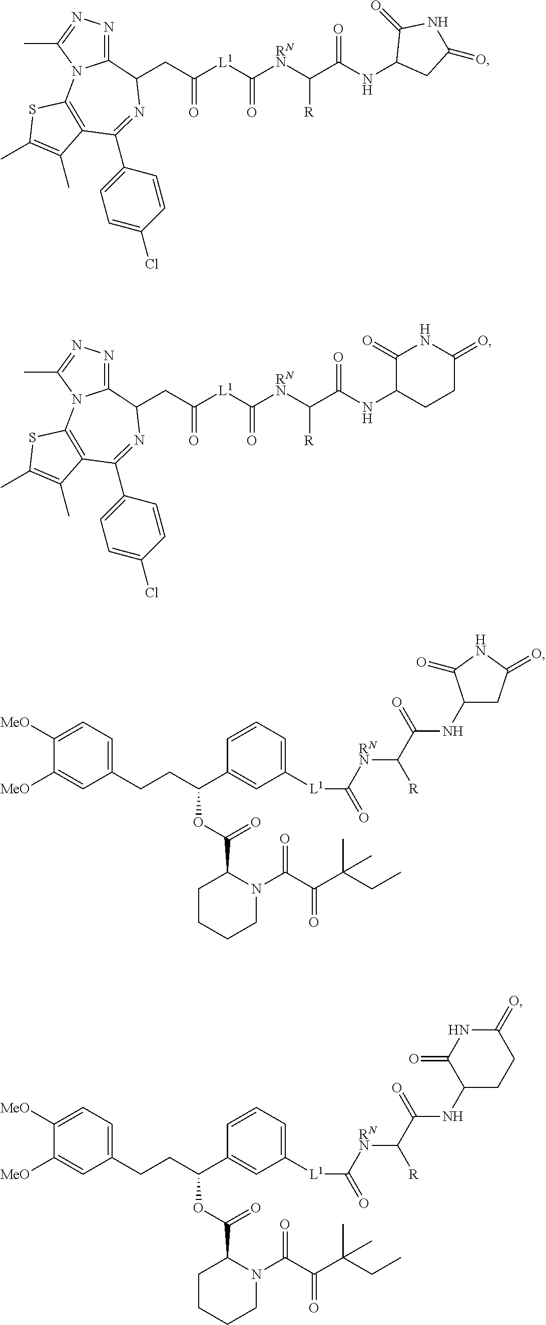

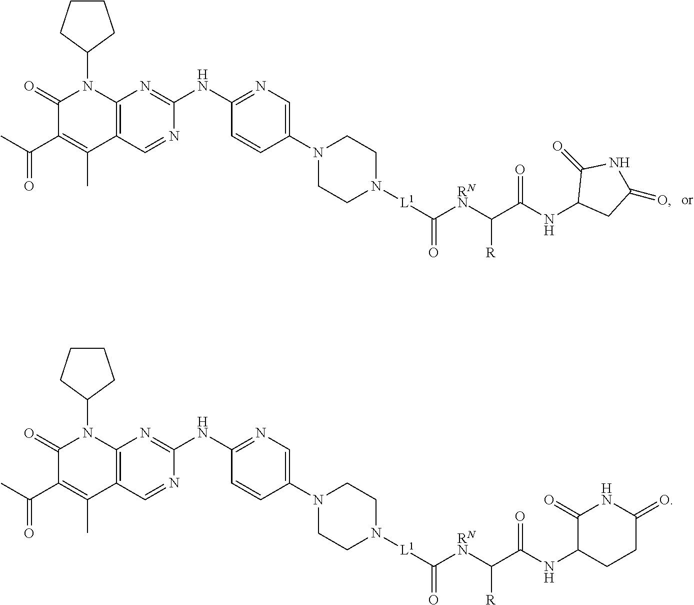

In some embodiments, the compound of Formula (I′) or (I) is of the formula:

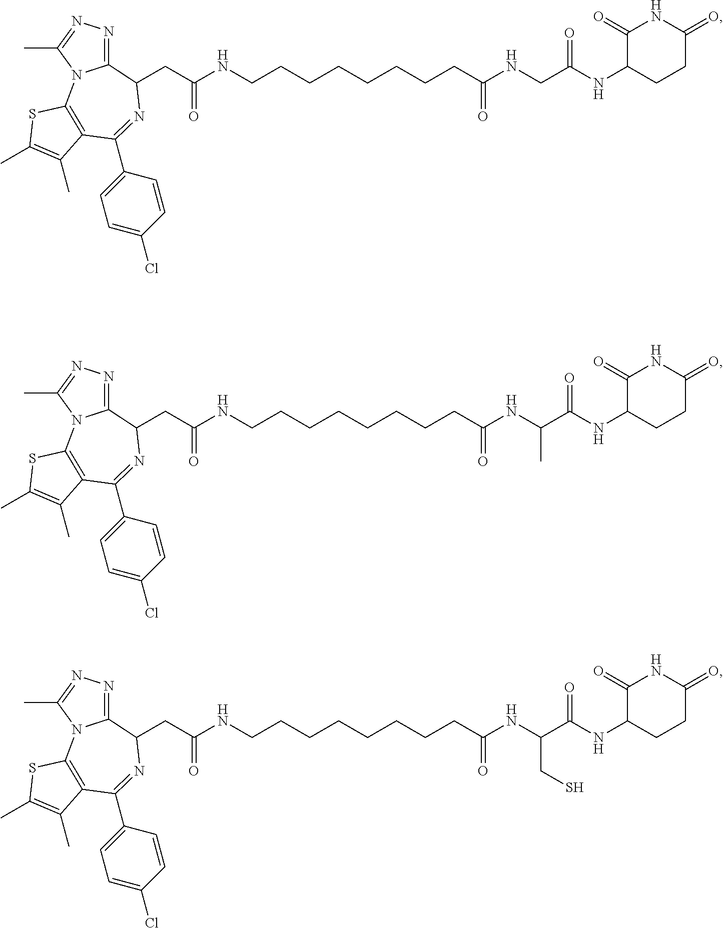 [SMILES: Cc1sc2c(c1C)C(c1ccc(Cl)cc1)=NC(CC(=O)NCCCCCCCCC(=O)NCC(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(C)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CS)C(=O)NC1CCC(=O)NC1=O)c1nnc(C)n1-2]

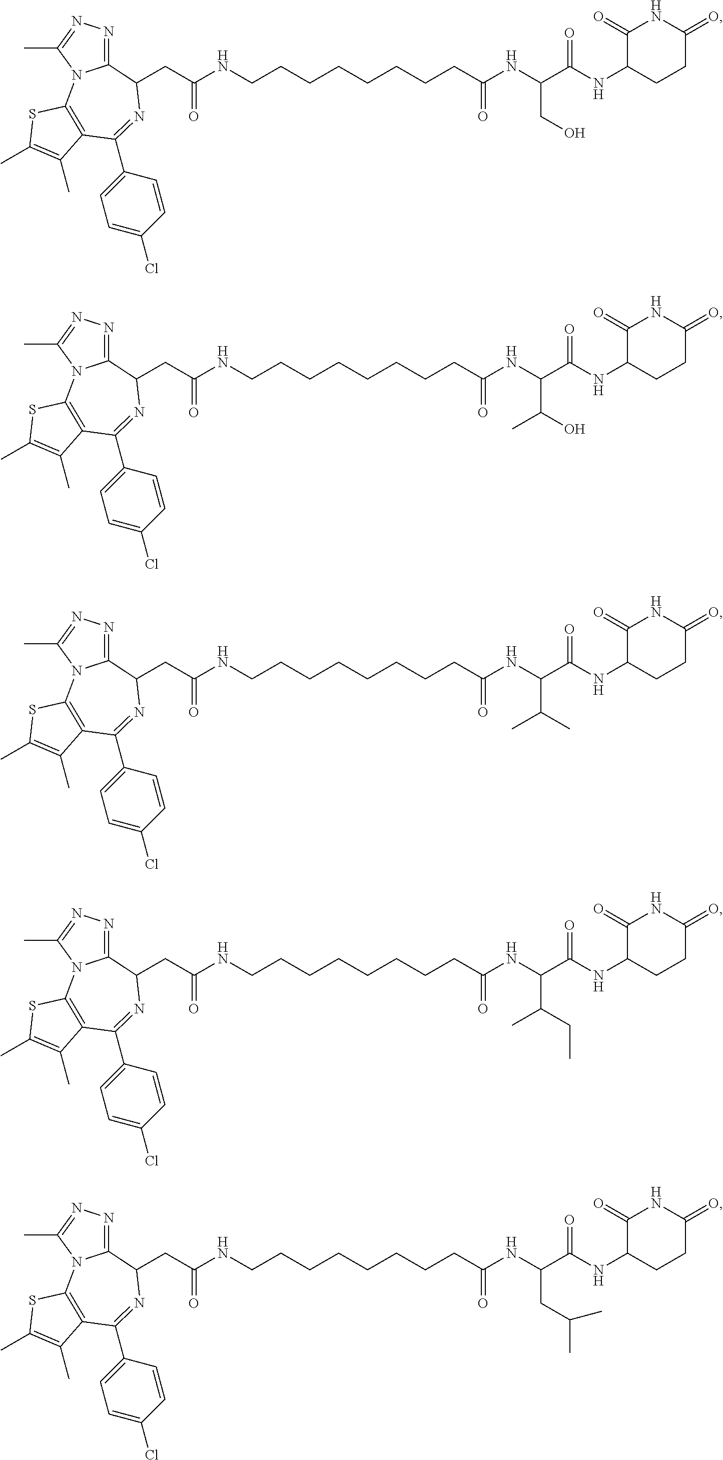 [SMILES: Cc1sc2c(c1C)C(c1ccc(Cl)cc1)=NC(CC(=O)NCCCCCCCCC(=O)NC(CO)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(C(=O)NC1CCC(=O)NC1=O)C(C)O)c1nnc(C)n1-2 ; Cc1sc2c(c1C)C(c1ccc(Cl)cc1)=NC(CC(=O)NCCCCCCCCC(=O)NC(C(=O)NC1CCC(=O)NC1=O)C(C)C)c1nnc(C)n1-2 ; CCC(C)C(NC(=O)CCCCCCCCNC(=O)CC1N=C(c2ccc(Cl)cc2)c2c(sc(C)c2C)-n2c(C)nnc21)C(=O)NC1CCC(=O)NC1=O ; Cc1sc2c(c1C)C(c1ccc(Cl)cc1)=NC(CC(=O)NCCCCCCCCC(=O)NC(CC(C)C)C(=O)NC1CCC(=O)NC1=O)c1nnc(C)n1-2]

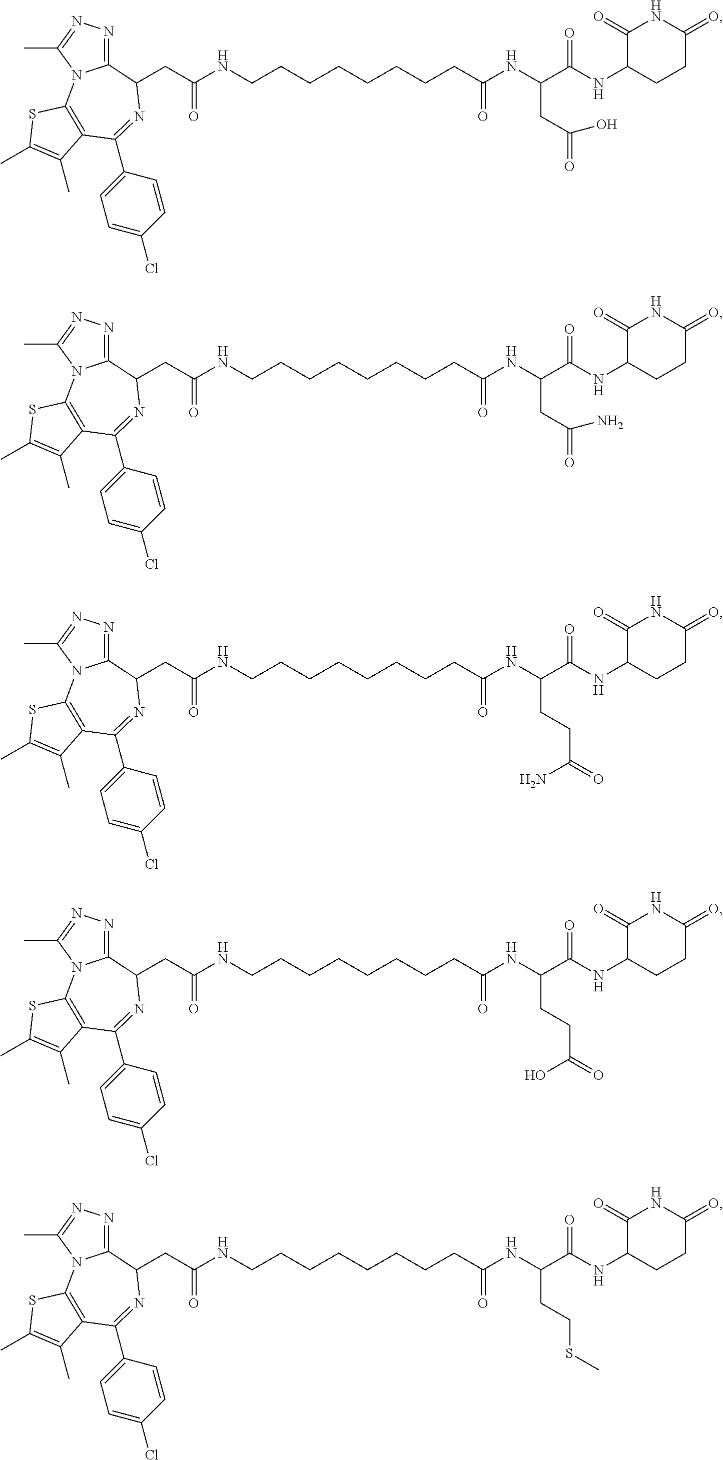 [SMILES: Cc1sc2c(c1C)C(c1ccc(Cl)cc1)=NC(CC(=O)NCCCCCCCCC(=O)NC(CC(=O)O)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CC(N)=O)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CCC(N)=O)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CCC(=O)O)C(=O)NC1CCC(=O)NC1=O)c1nnc(C)n1-2 ; CSCCC(NC(=O)CCCCCCCCNC(=O)CC1N=C(c2ccc(Cl)cc2)c2c(sc(C)c2C)-n2c(C)nnc21)C(=O)NC1CCC(=O)NC1=O]

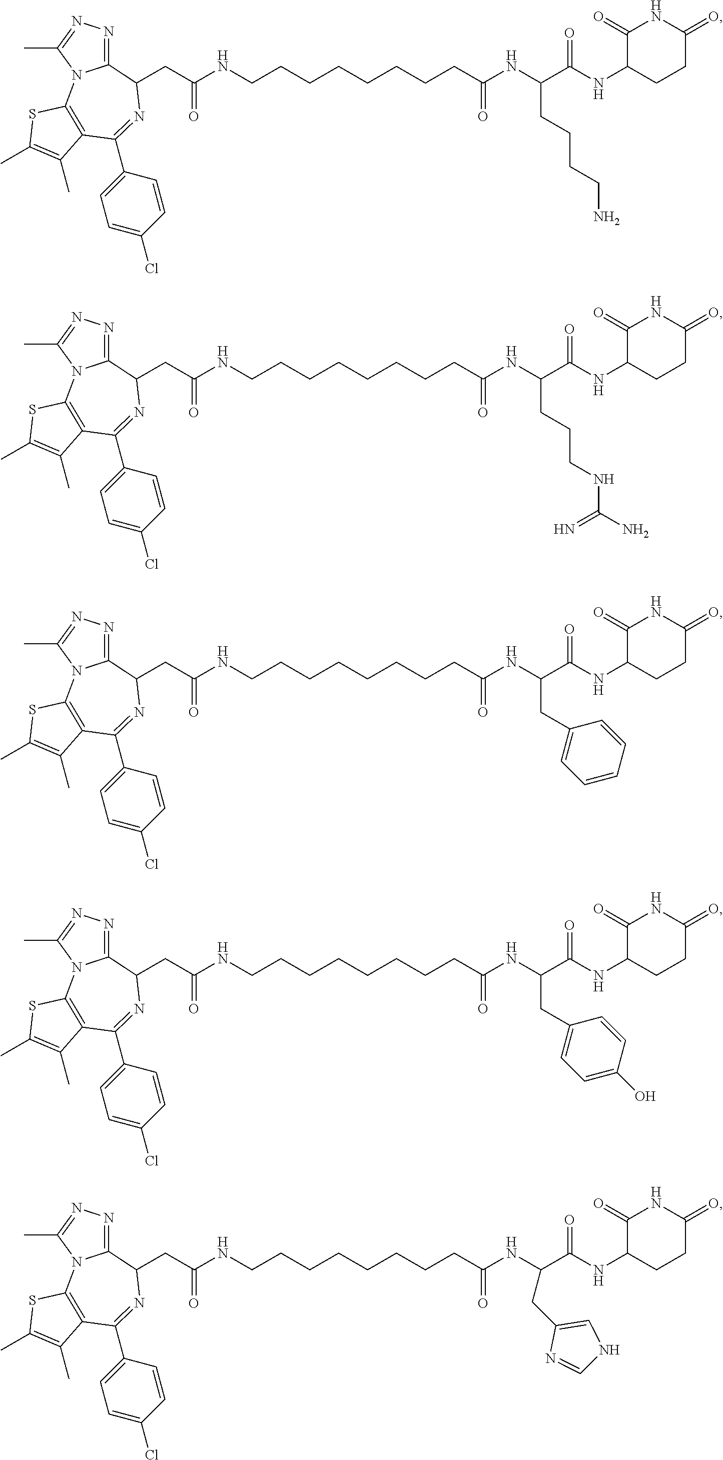 [SMILES: Cc1sc2c(c1C)C(c1ccc(Cl)cc1)=NC(CC(=O)NCCCCCCCCC(=O)NC(CCCCN)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CCCNC(=N)N)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(Cc1ccccc1)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(Cc1ccc(O)cc1)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(Cc1c[nH]cn1)C(=O)NC1CCC(=O)NC1=O)c1nnc(C)n1-2]

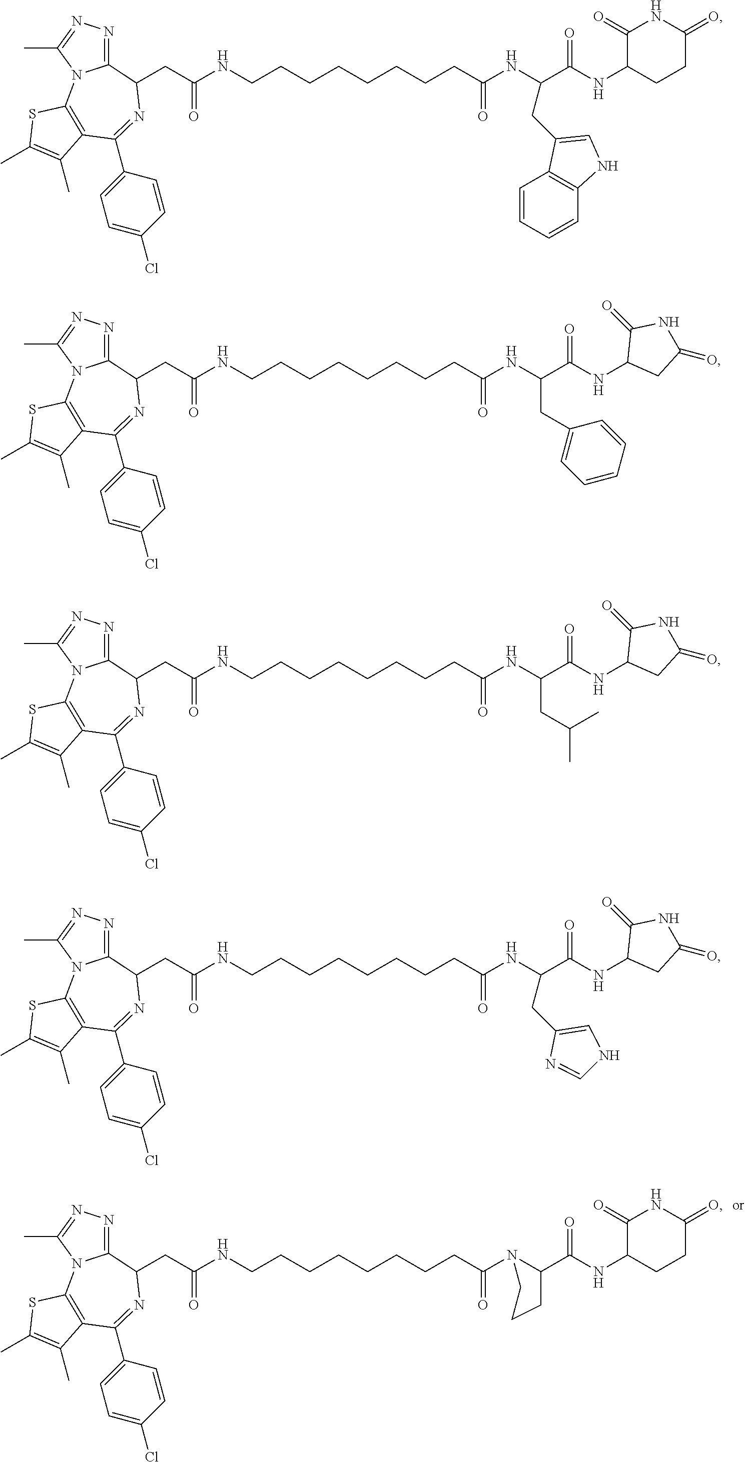 [SMILES: Cc1sc2c(c1C)C(c1ccc(Cl)cc1)=NC(CC(=O)NCCCCCCCCC(=O)NC(Cc1c[nH]c3ccccc13)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(Cc1ccccc1)C(=O)NC1CC(=O)NC1=O)c1nnc(C)n1-2 ; Cc1sc2c(c1C)C(c1ccc(Cl)cc1)=NC(CC(=O)NCCCCCCCCC(=O)NC(CC(C)C)C(=O)NC1CC(=O)NC1=O)c1nnc(C)n1-2 ; Cc1sc2c(c1C)C(c1ccc(Cl)cc1)=NC(CC(=O)NCCCCCCCCC(=O)NC(Cc1c[nH]cn1)C(=O)NC1CC(=O)NC1=O)c1nnc(C)n1-2 ; Cc1sc2c(c1C)C(c1ccc(Cl)cc1)=NC(CC(=O)NCCCCCCCCC(=O)N1CCCC1C(=O)NC1CCC(=O)NC1=O)c1nnc(C)n1-2]

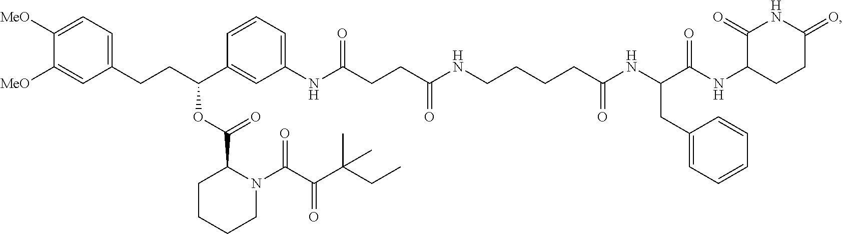 [SMILES: CCC(C)(C)C(=O)C(=O)N1CCCC[C@H]1C(=O)O[C@H](CCc1ccc(OC)c(OC)c1)c1cccc(NC(=O)CCC(=O)NCCCCC(=O)NC(Cc2ccccc2)C(=O)NC2CCC(=O)NC2=O)c1]

or a pharmaceutically acceptable salt or tautomer thereof.

In some embodiments, a compound of Formula (I′) or (I) is of the formula:

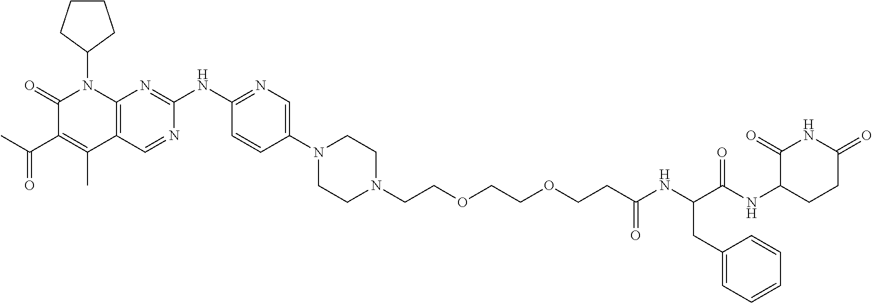 [SMILES: CC(=O)c1c(C)c2cnc(Nc3ccc(N4CCN(CCOCCOCCC(=O)NC(Cc5ccccc5)C(=O)NC5CCC(=O)NC5=O)CC4)cn3)nc2n(C2CCCC2)c1=O]

In some embodiments, a compound of Formula (I′) or (I) is of the formula:

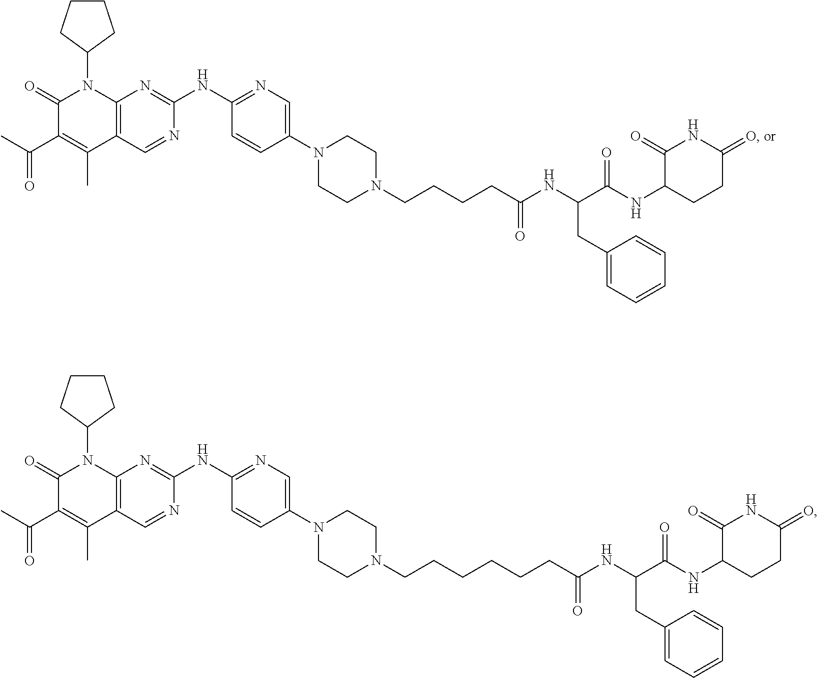 [SMILES: CC(=O)c1c(C)c2cnc(Nc3ccc(N4CCN(CCCCC(=O)NC(Cc5ccccc5)C(=O)NC5CCC(=O)NC5=O)CC4)cn3)nc2n(C2CCCC2)c1=O ; CC(=O)c1c(C)c2cnc(Nc3ccc(N4CCN(CCCCCCC(=O)NC(Cc5ccccc5)C(=O)NC5CCC(=O)NC5=O)CC4)cn3)nc2n(C2CCCC2)c1=O]

or a pharmaceutically acceptable salt or tautomer thereof.

In some embodiments, the compound of Formula (I′) or (I) is of the formula:

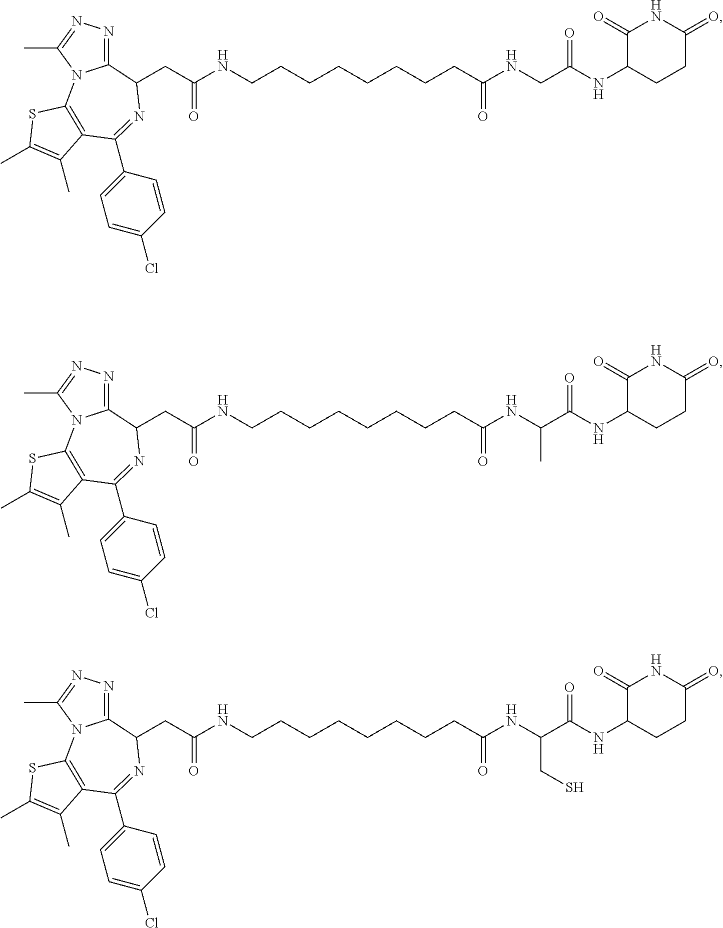 [SMILES: Cc1sc2c(c1C)C(c1ccc(Cl)cc1)=NC(CC(=O)NCCCCCCCCC(=O)NCC(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(C)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CS)C(=O)NC1CCC(=O)NC1=O)c1nnc(C)n1-2]

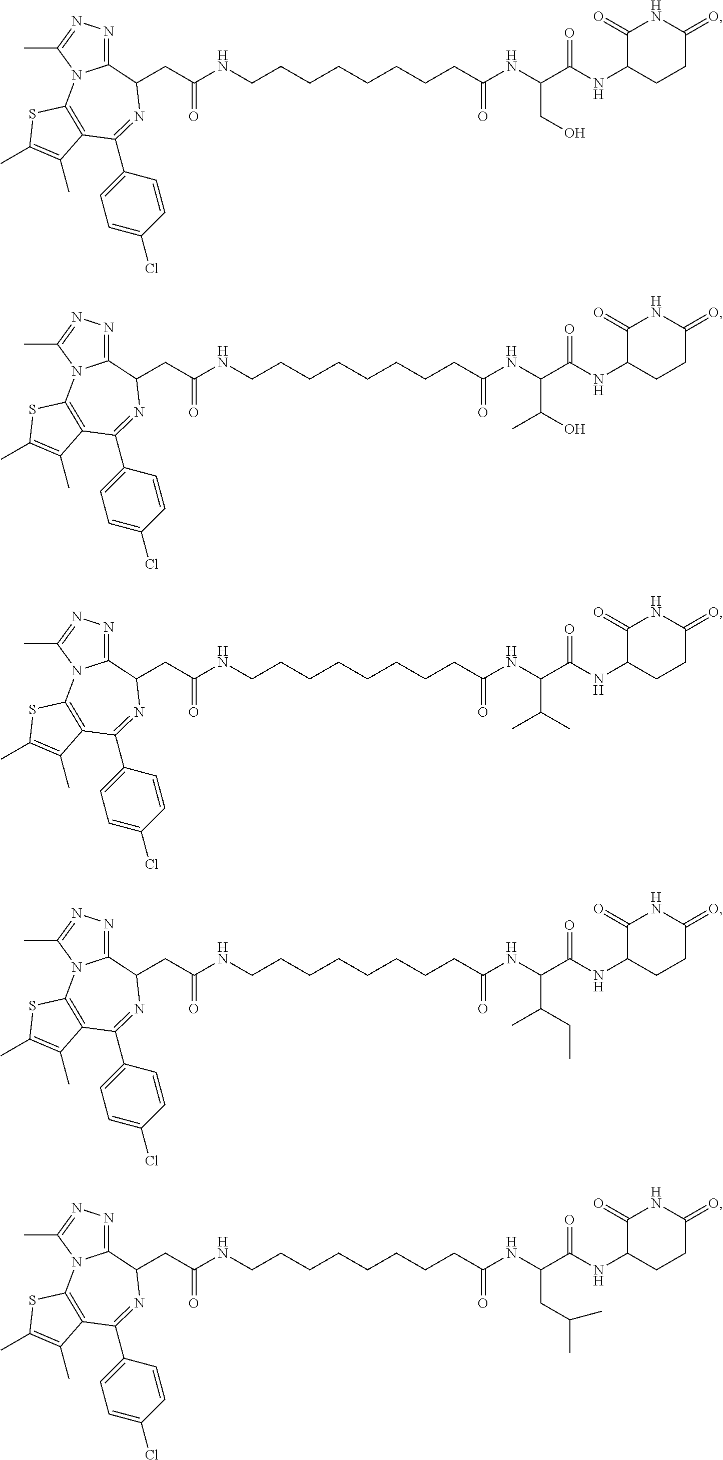 [SMILES: Cc1sc2c(c1C)C(c1ccc(Cl)cc1)=NC(CC(=O)NCCCCCCCCC(=O)NC(CO)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(C(=O)NC1CCC(=O)NC1=O)C(C)O)c1nnc(C)n1-2 ; Cc1sc2c(c1C)C(c1ccc(Cl)cc1)=NC(CC(=O)NCCCCCCCCC(=O)NC(C(=O)NC1CCC(=O)NC1=O)C(C)C)c1nnc(C)n1-2 ; CCC(C)C(NC(=O)CCCCCCCCNC(=O)CC1N=C(c2ccc(Cl)cc2)c2c(sc(C)c2C)-n2c(C)nnc21)C(=O)NC1CCC(=O)NC1=O ; Cc1sc2c(c1C)C(c1ccc(Cl)cc1)=NC(CC(=O)NCCCCCCCCC(=O)NC(CC(C)C)C(=O)NC1CCC(=O)NC1=O)c1nnc(C)n1-2]

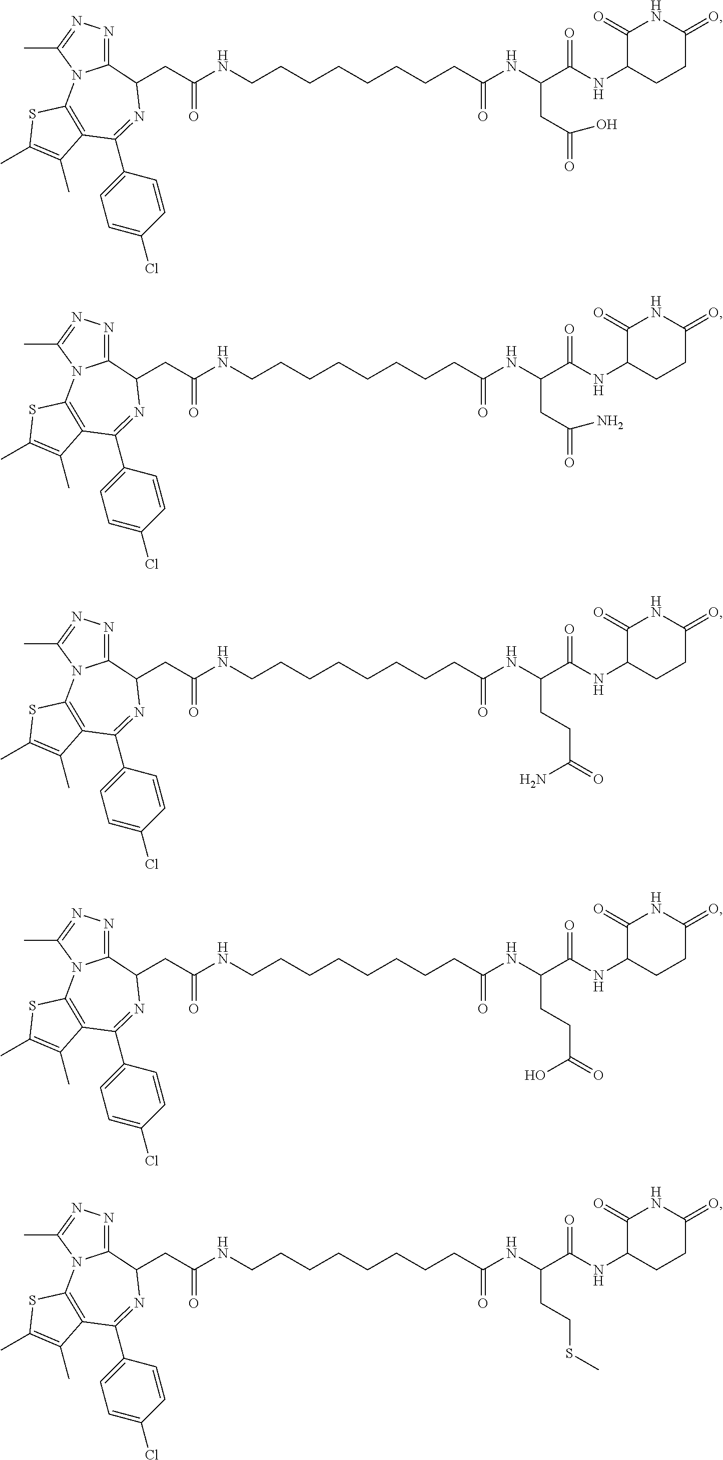 [SMILES: Cc1sc2c(c1C)C(c1ccc(Cl)cc1)=NC(CC(=O)NCCCCCCCCC(=O)NC(CC(=O)O)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CC(N)=O)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CCC(N)=O)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CCC(=O)O)C(=O)NC1CCC(=O)NC1=O)c1nnc(C)n1-2 ; CSCCC(NC(=O)CCCCCCCCNC(=O)CC1N=C(c2ccc(Cl)cc2)c2c(sc(C)c2C)-n2c(C)nnc21)C(=O)NC1CCC(=O)NC1=O]

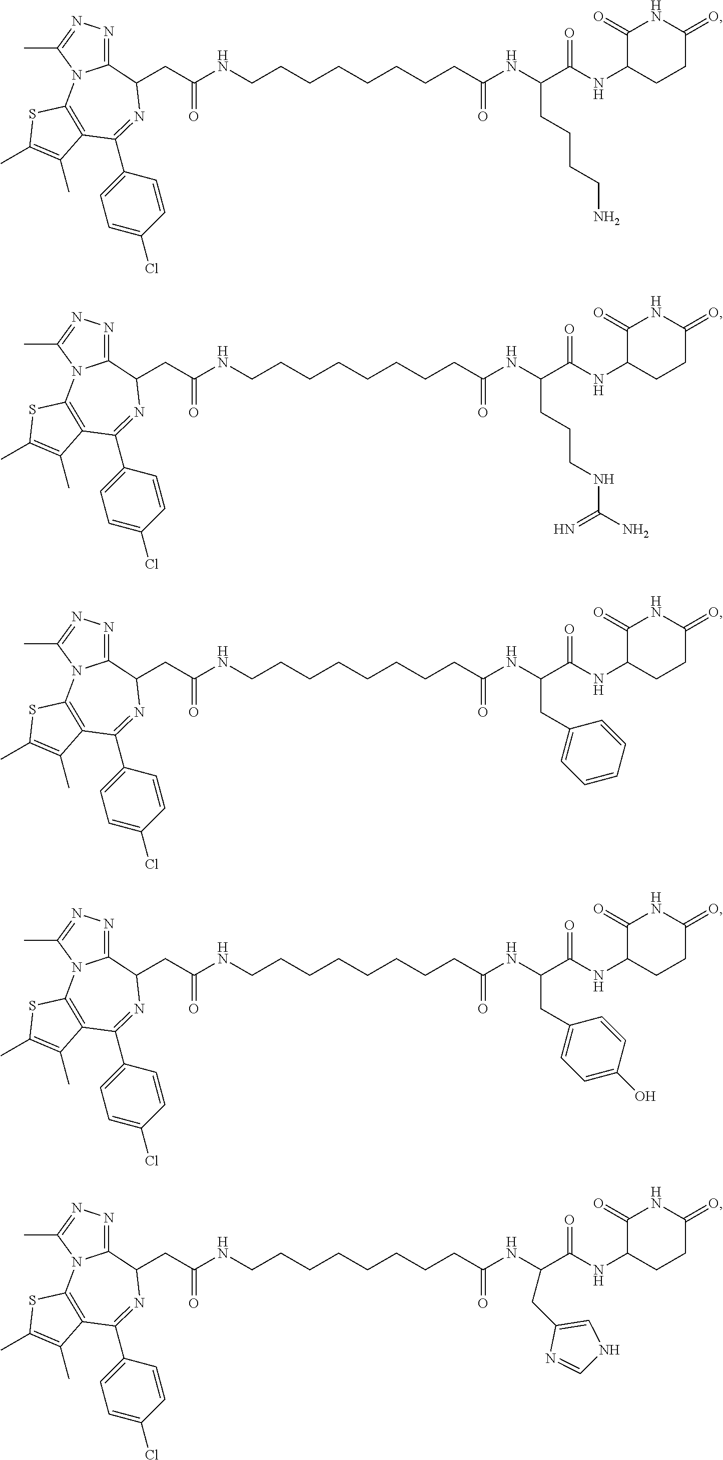 [SMILES: Cc1sc2c(c1C)C(c1ccc(Cl)cc1)=NC(CC(=O)NCCCCCCCCC(=O)NC(CCCCN)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CCCNC(=N)N)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(Cc1ccccc1)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(Cc1ccc(O)cc1)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(Cc1c[nH]cn1)C(=O)NC1CCC(=O)NC1=O)c1nnc(C)n1-2]

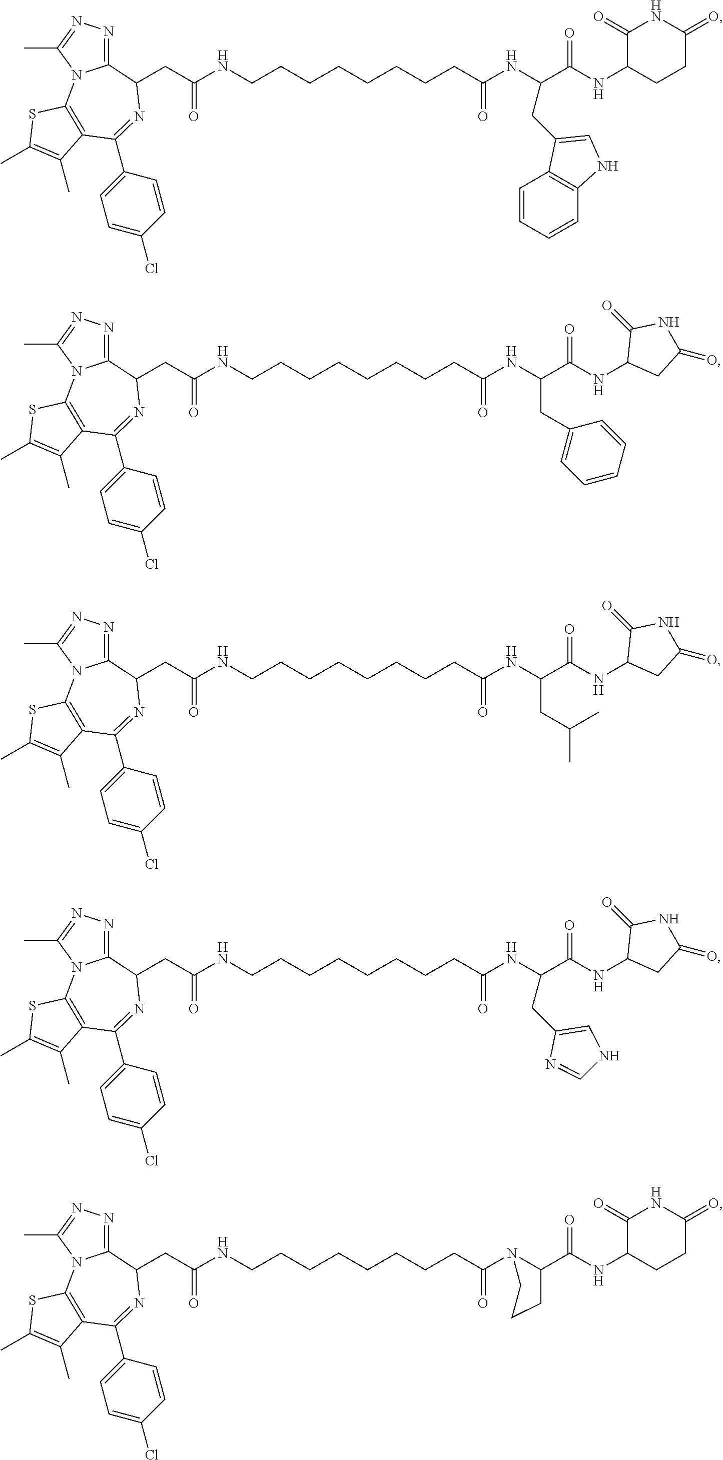 [SMILES: Cc1sc2c(c1C)C(c1ccc(Cl)cc1)=NC(CC(=O)NCCCCCCCCC(=O)NC(Cc1c[nH]c3ccccc13)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(Cc1ccccc1)C(=O)NC1CC(=O)NC1=O)c1nnc(C)n1-2 ; Cc1sc2c(c1C)C(c1ccc(Cl)cc1)=NC(CC(=O)NCCCCCCCCC(=O)NC(CC(C)C)C(=O)NC1CC(=O)NC1=O)c1nnc(C)n1-2 ; Cc1sc2c(c1C)C(c1ccc(Cl)cc1)=NC(CC(=O)NCCCCCCCCC(=O)NC(Cc1c[nH]cn1)C(=O)NC1CC(=O)NC1=O)c1nnc(C)n1-2 ; Cc1sc2c(c1C)C(c1ccc(Cl)cc1)=NC(CC(=O)NCCCCCCCCC(=O)N1CCCC1C(=O)NC1CCC(=O)NC1=O)c1nnc(C)n1-2]

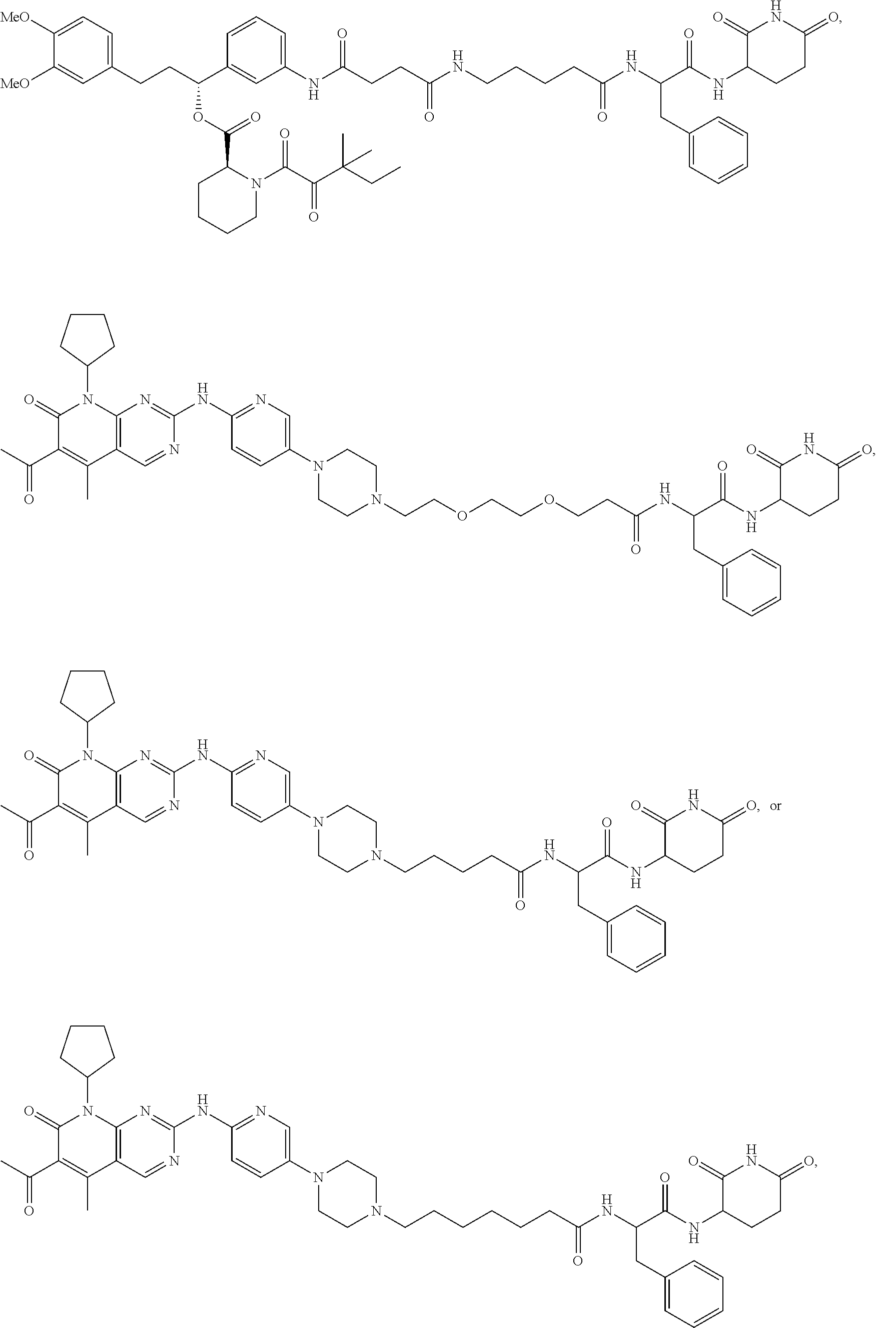 [SMILES: CCC(C)(C)C(=O)C(=O)N1CCCC[C@H]1C(=O)O[C@H](CCc1ccc(OC)c(OC)c1)c1cccc(NC(=O)CCC(=O)NCCCCC(=O)NC(Cc2ccccc2)C(=O)NC2CCC(=O)NC2=O)c1 ; CC(=O)c1c(C)c2cnc(Nc3ccc(N4CCN(CCOCCOCCC(=O)NC(Cc5ccccc5)C(=O)NC5CCC(=O)NC5=O)CC4)cn3)nc2n(C2CCCC2)c1=O ; CC(=O)c1c(C)c2cnc(Nc3ccc(N4CCN(CCCCC(=O)NC(Cc5ccccc5)C(=O)NC5CCC(=O)NC5=O)CC4)cn3)nc2n(C2CCCC2)c1=O ; CC(=O)c1c(C)c2cnc(Nc3ccc(N4CCN(CCCCCCC(=O)NC(Cc5ccccc5)C(=O)NC5CCC(=O)NC5=O)CC4)cn3)nc2n(C2CCCC2)c1=O]

or a pharmaceutically acceptable salt or tautomer thereof.
In some embodiments, a compound of Formula (I′) or (I) lacks a protein targeting moiety (i.e., B).

In some embodiments, a compound of Formula (I′) or (I) is of the formula:

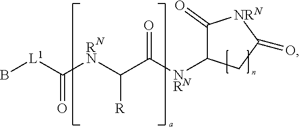

wherein

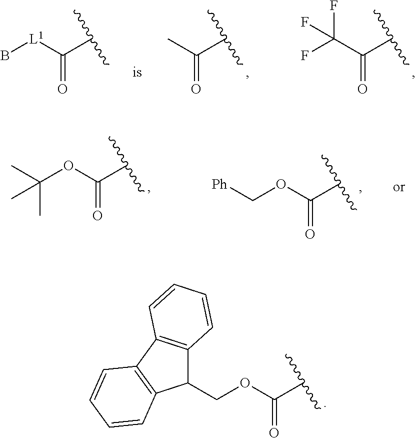

In some embodiments,

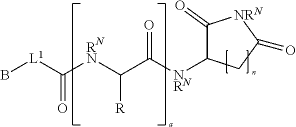

is of the formula:

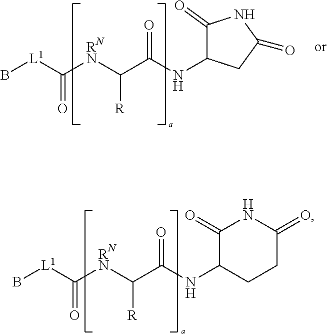

wherein

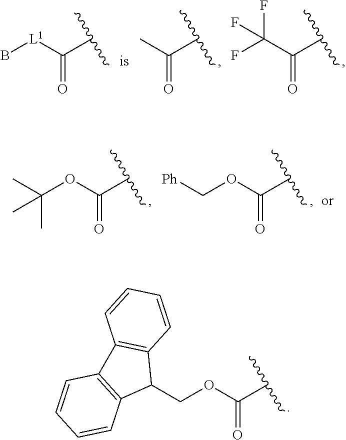

In some embodiments,

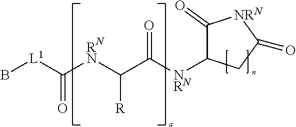

is of the formula:

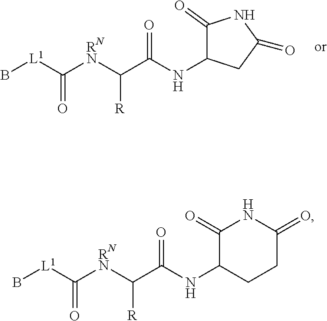

wherein

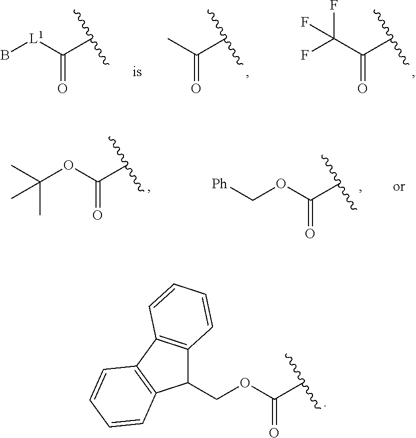

In some embodiments, a compound of Formula (I′) or (I) is of the formula:

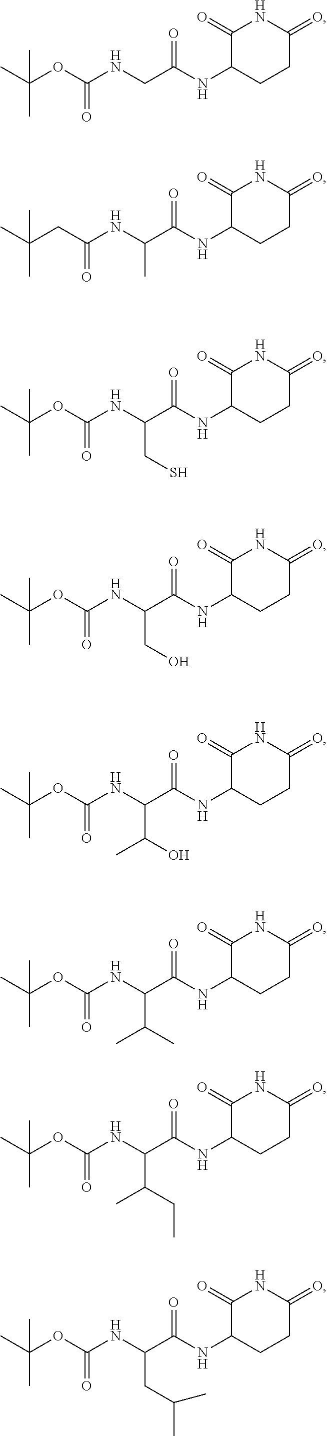

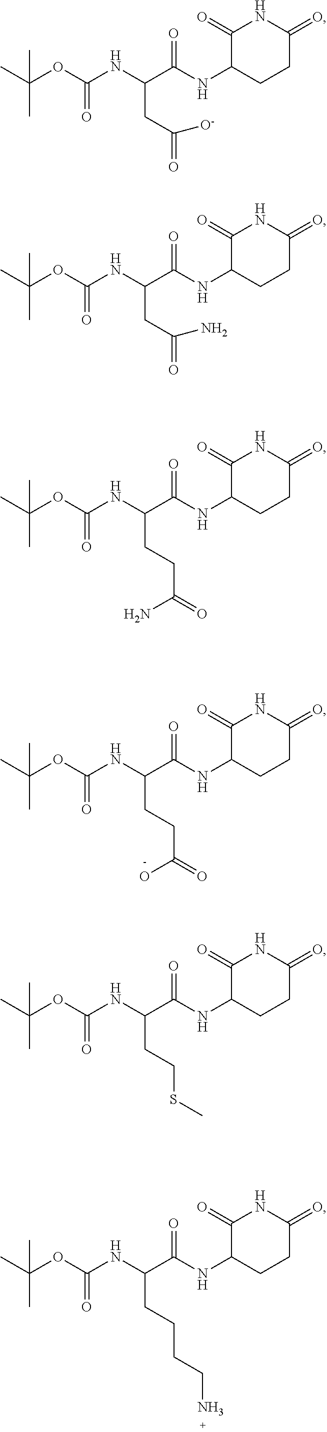 [SMILES: CC(C)(C)OC(=O)NC(CC(=O)[O-])C(=O)NC1CCC(=O)NC1=O ; CC(C)(C)OC(=O)NC(CC(N)=O)C(=O)NC1CCC(=O)NC1=O ; CC(C)(C)OC(=O)NC(CCC(N)=O)C(=O)NC1CCC(=O)NC1=O ; CC(C)(C)OC(=O)NC(CCC(=O)O)C(=O)NC1CCC(=O)NC1=O ; CSCCC(NC(=O)OC(C)(C)C)C(=O)NC1CCC(=O)NC1=O ; CC(C)(C)OC(=O)NC(CCCCN)C(=O)NC1CCC(=O)NC1=O]

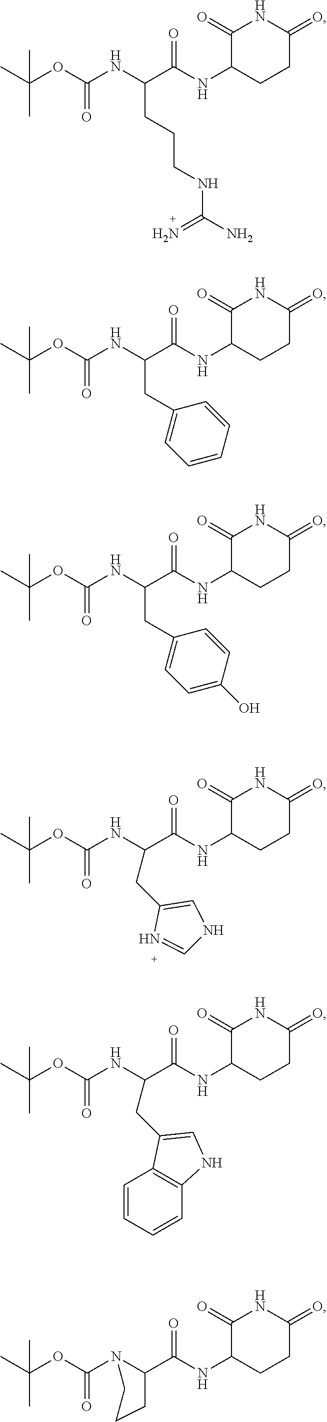 [SMILES: CC(C)(C)OC(=O)NC(CCCNC(=N)N)C(=O)NC1CCC(=O)NC1=O ; CC(C)(C)OC(=O)NC(Cc1ccccc1)C(=O)NC1CCC(=O)NC1=O ; CC(C)(C)OC(=O)NC(Cc1ccc(O)cc1)C(=O)NC1CCC(=O)NC1=O ; CC(C)(C)OC(=O)NC(Cc1c[nH]cn1)C(=O)NC1CCC(=O)NC1=O ; CC(C)(C)OC(=O)NC(Cc1c[nH]c2ccccc12)C(=O)NC1CCC(=O)NC1=O ; CC(C)(C)OC(=O)N1CCCC1C(=O)NC1CCC(=O)NC1=O]

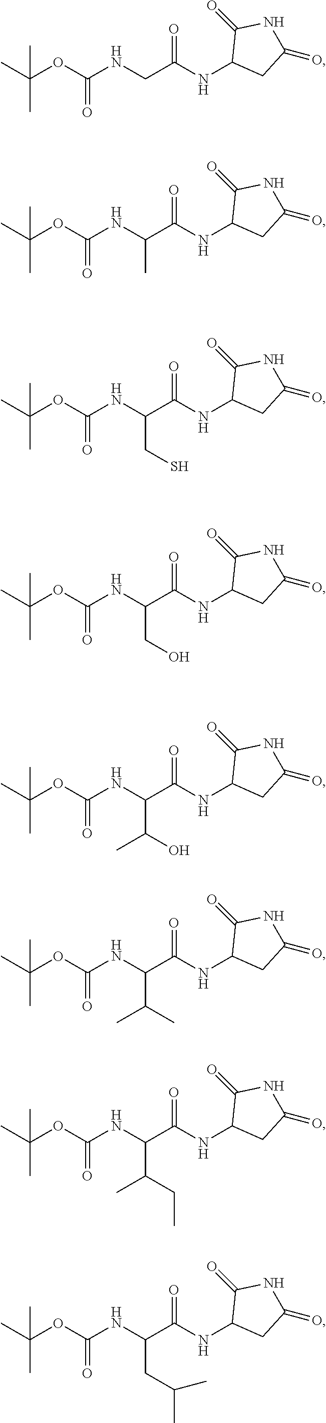

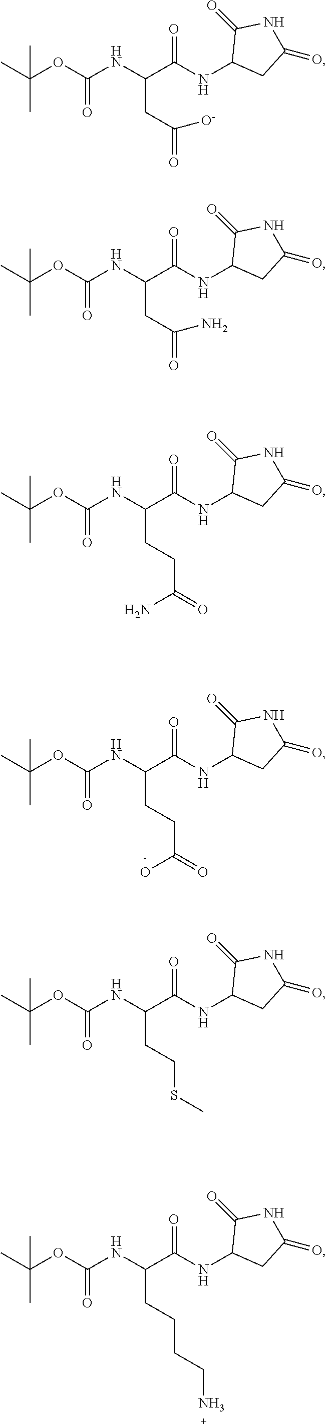 [SMILES: CC(C)(C)OC(=O)NC(CC(=O)[O-])C(=O)NC1CC(=O)NC1=O ; CC(C)(C)OC(=O)NC(CC(N)=O)C(=O)NC1CC(=O)NC1=O ; CC(C)(C)OC(=O)NC(CCC(N)=O)C(=O)NC1CC(=O)NC1=O ; CC(C)(C)OC(=O)NC(CCC(=O)O)C(=O)NC1CC(=O)NC1=O ; CSCCC(NC(=O)OC(C)(C)C)C(=O)NC1CC(=O)NC1=O ; CC(C)(C)OC(=O)NC(CCCCN)C(=O)NC1CC(=O)NC1=O]

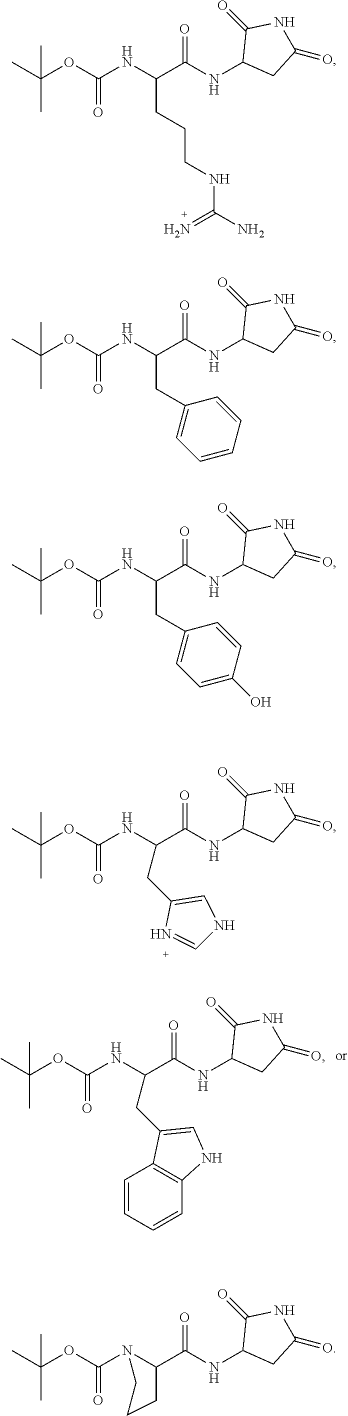 [SMILES: CC(C)(C)OC(=O)NC(CCCNC(=N)N)C(=O)NC1CC(=O)NC1=O ; CC(C)(C)OC(=O)NC(Cc1ccccc1)C(=O)NC1CC(=O)NC1=O ; CC(C)(C)OC(=O)NC(Cc1ccc(O)cc1)C(=O)NC1CC(=O)NC1=O ; CC(C)(C)OC(=O)NC(Cc1c[nH]cn1)C(=O)NC1CC(=O)NC1=O ; CC(C)(C)OC(=O)NC(Cc1c[nH]c2ccccc12)C(=O)NC1CC(=O)NC1=O ; CC(C)(C)OC(=O)N1CCCC1C(=O)NC1CC(=O)NC1=O]

In some embodiments, a compound of Formula (I′) or (I) is of the formula:

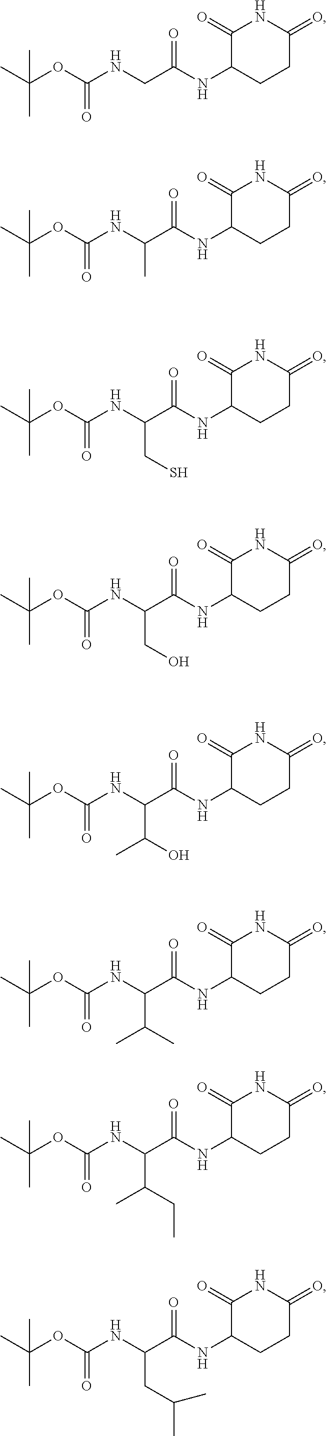

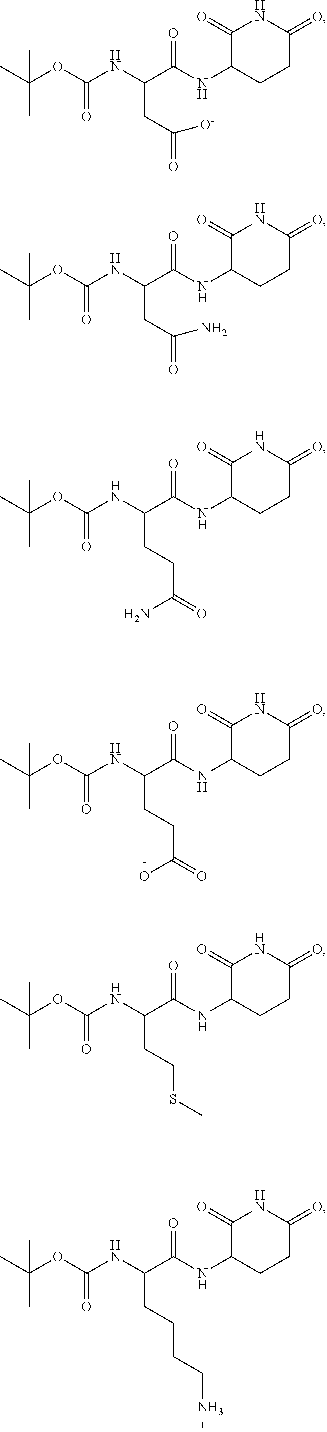 [SMILES: CC(C)(C)OC(=O)NC(CC(=O)[O-])C(=O)NC1CCC(=O)NC1=O ; CC(C)(C)OC(=O)NC(CC(N)=O)C(=O)NC1CCC(=O)NC1=O ; CC(C)(C)OC(=O)NC(CCC(N)=O)C(=O)NC1CCC(=O)NC1=O ; CC(C)(C)OC(=O)NC(CCC(=O)O)C(=O)NC1CCC(=O)NC1=O ; CSCCC(NC(=O)OC(C)(C)C)C(=O)NC1CCC(=O)NC1=O ; CC(C)(C)OC(=O)NC(CCCCN)C(=O)NC1CCC(=O)NC1=O]

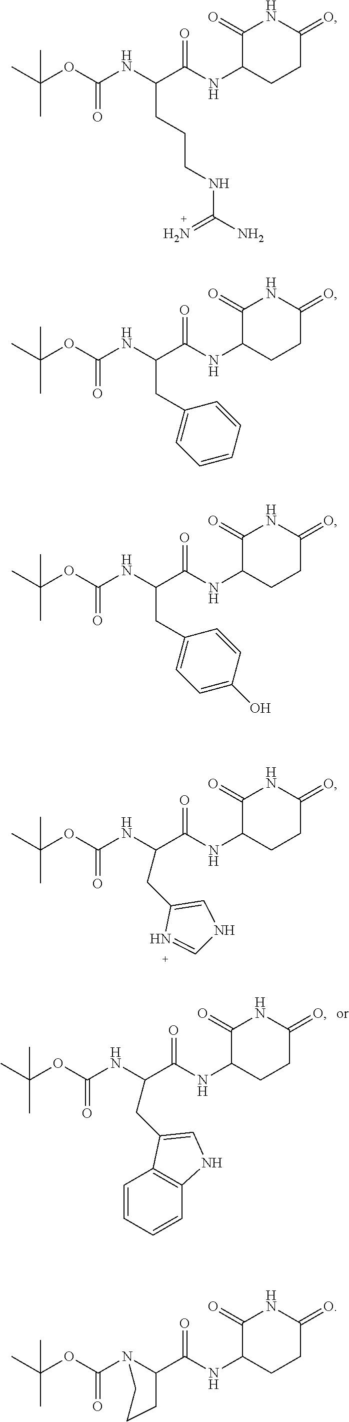 [SMILES: CC(C)(C)OC(=O)NC(CCCNC(=N)N)C(=O)NC1CCC(=O)NC1=O ; CC(C)(C)OC(=O)NC(Cc1ccccc1)C(=O)NC1CCC(=O)NC1=O ; CC(C)(C)OC(=O)NC(Cc1ccc(O)cc1)C(=O)NC1CCC(=O)NC1=O ; CC(C)(C)OC(=O)NC(Cc1c[nH]cn1)C(=O)NC1CCC(=O)NC1=O ; CC(C)(C)OC(=O)NC(Cc1c[nH]c2ccccc12)C(=O)NC1CCC(=O)NC1=O ; CC(C)(C)OC(=O)N1CCCC1C(=O)NC1CCC(=O)NC1=O]

In some embodiments, a compound of Formula (I′) or (I) is of the formula:

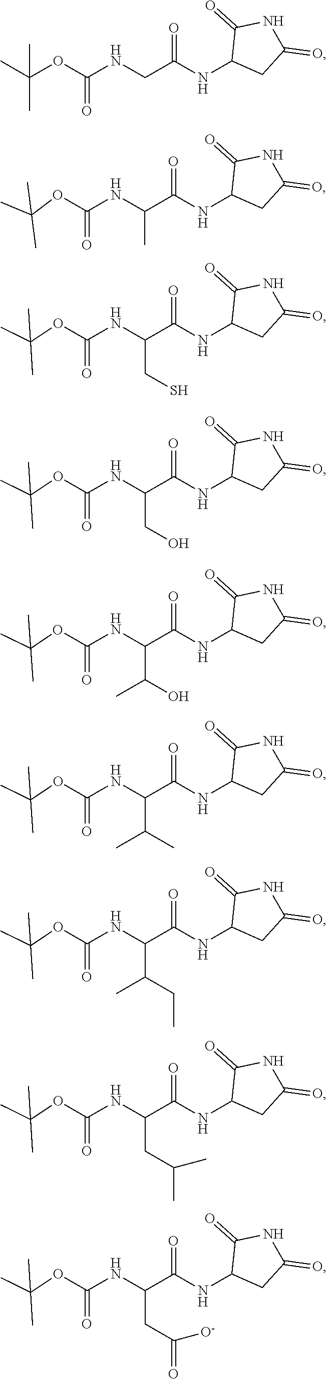

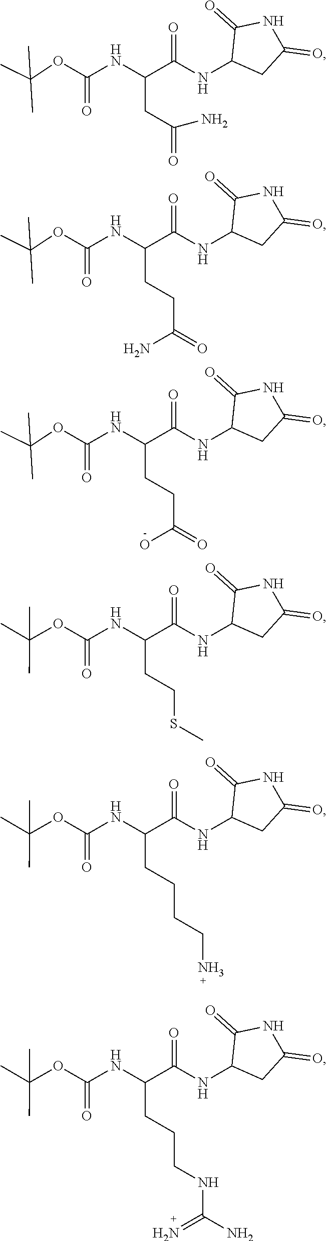

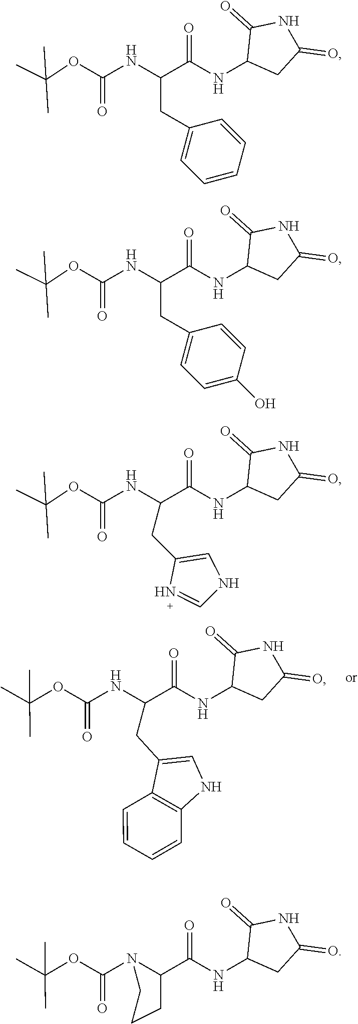

In some embodiments, a compound of Formula (I′) or (I) is of the formula:

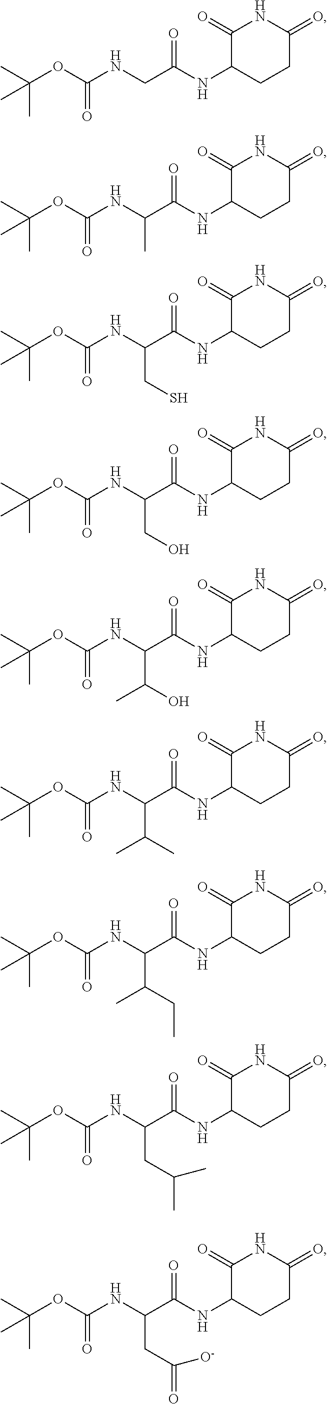

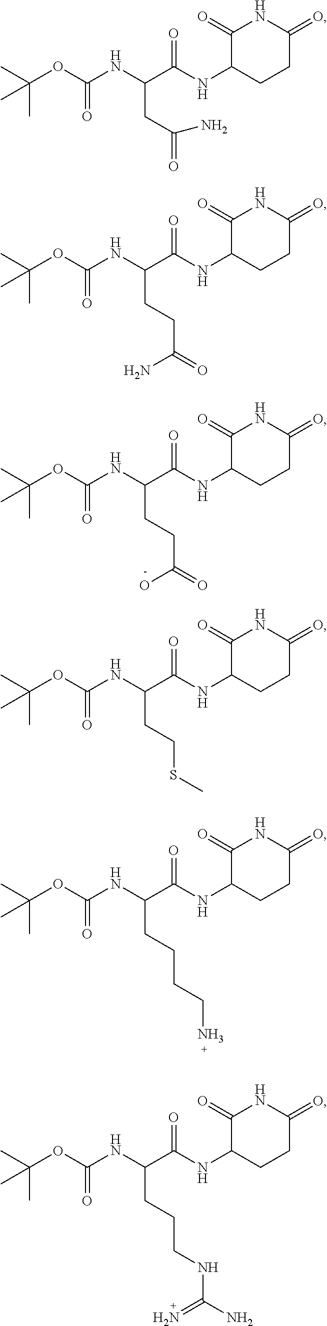

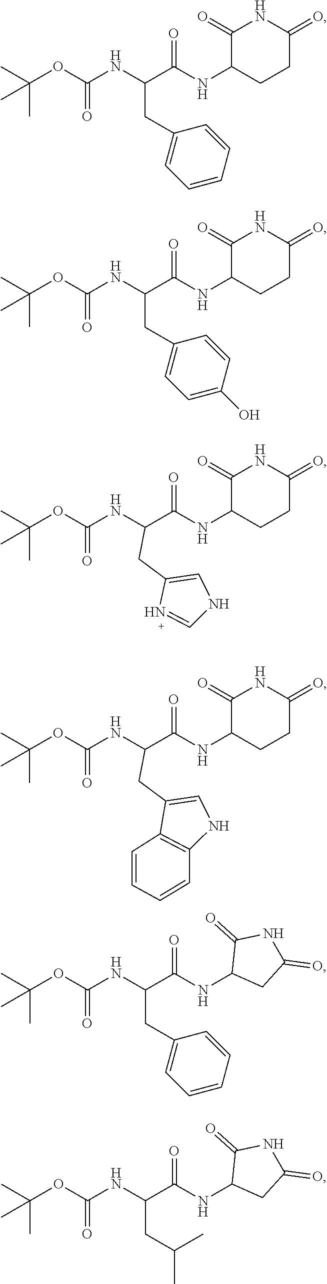

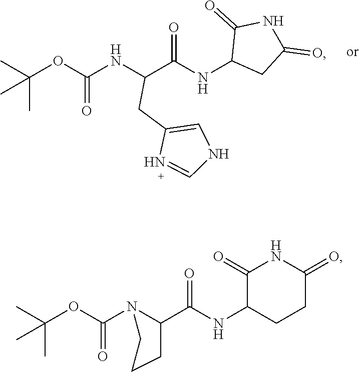 [SMILES: CC(C)(C)OC(=O)NC(Cc1c[nH]cn1)C(=O)NC1CC(=O)NC1=O ; "or" ; CC(C)(C)OC(=O)N1CCCC1C(=O)NC1CCC(=O)NC1=O]

or a pharmaceutically acceptable salt or tautomer thereof. In some embodiments, a compound of Formula (I′) is of the formula:

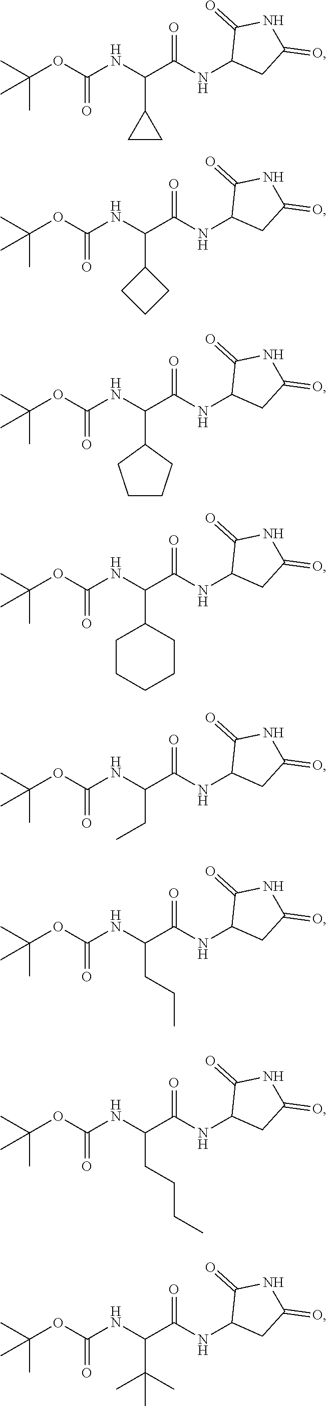

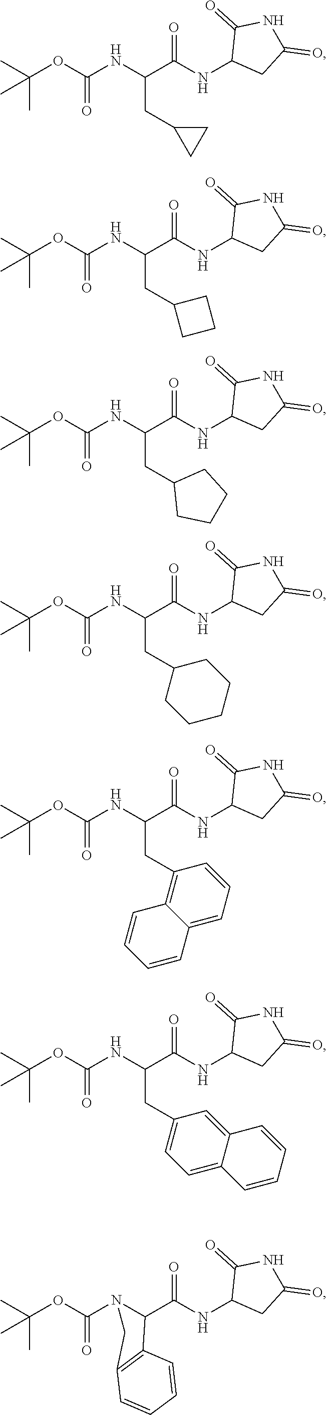

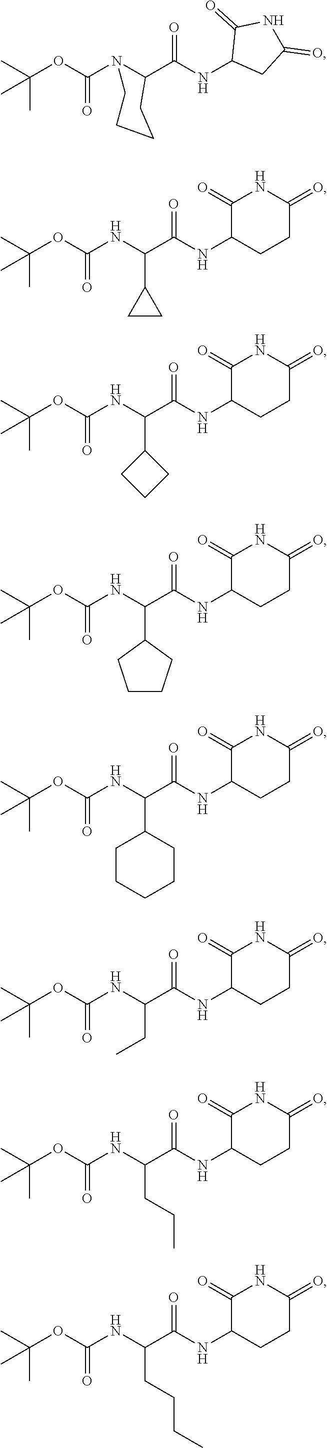

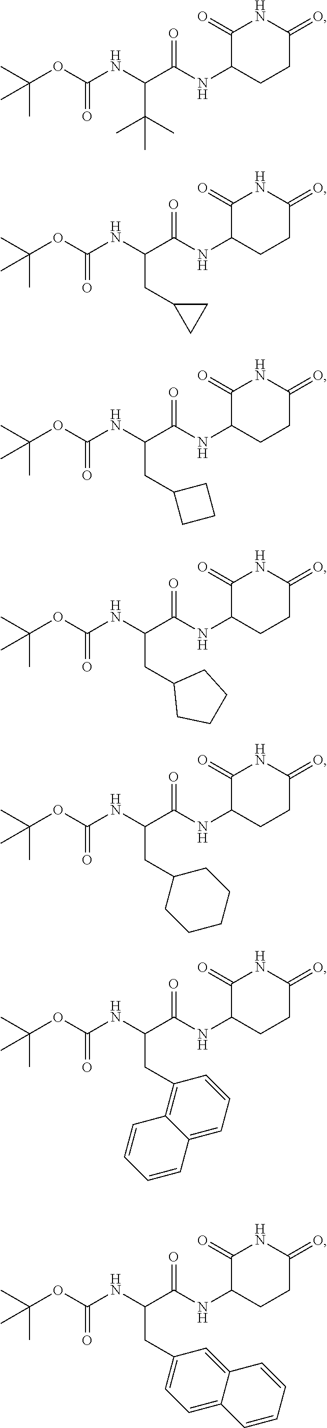

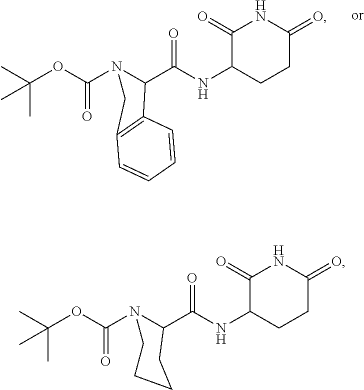 [SMILES: CC(C)(C)OC(=O)N1Cc2ccccc2C1C(=O)NC1CCC(=O)NC1=O ; "or" ; CC(C)(C)OC(=O)N1CCCCC1C(=O)NC1CCC(=O)NC1=O]

or a pharmaceutically acceptable salt or tautomer thereof. In some embodiments, a compound of Formula (I′) is of the formula:

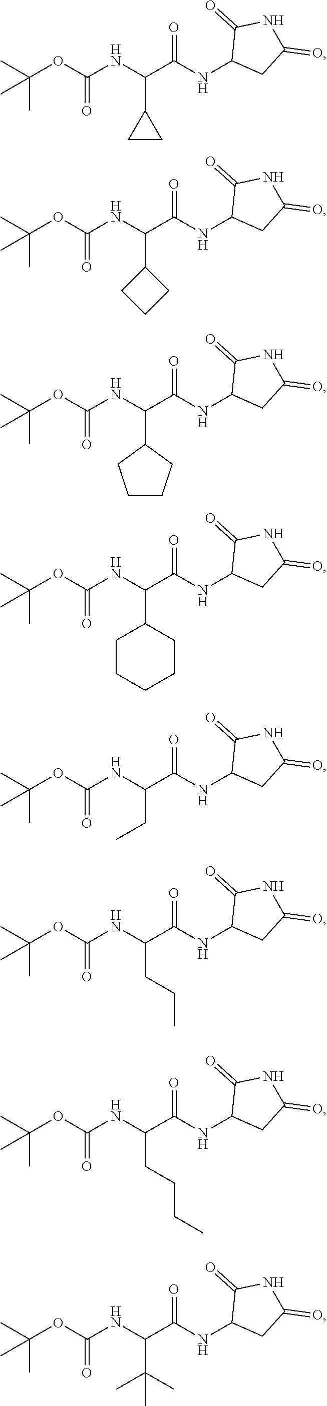

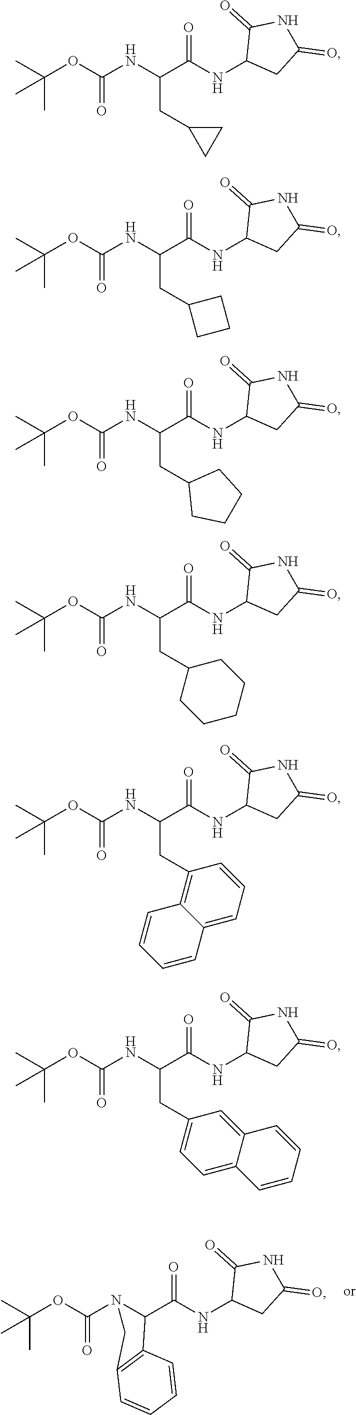

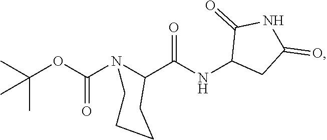 [SMILES: CC(C)(C)OC(=O)N1CCCCC1C(=O)NC1CC(=O)NC1=O]

or a pharmaceutically acceptable salt or tautomer thereof. In some embodiments, a compound of Formula (I′) is of the formula:

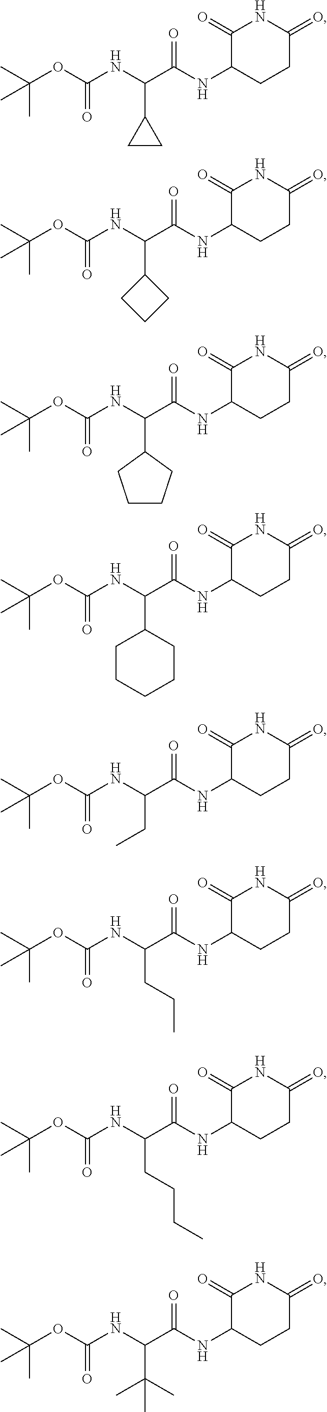

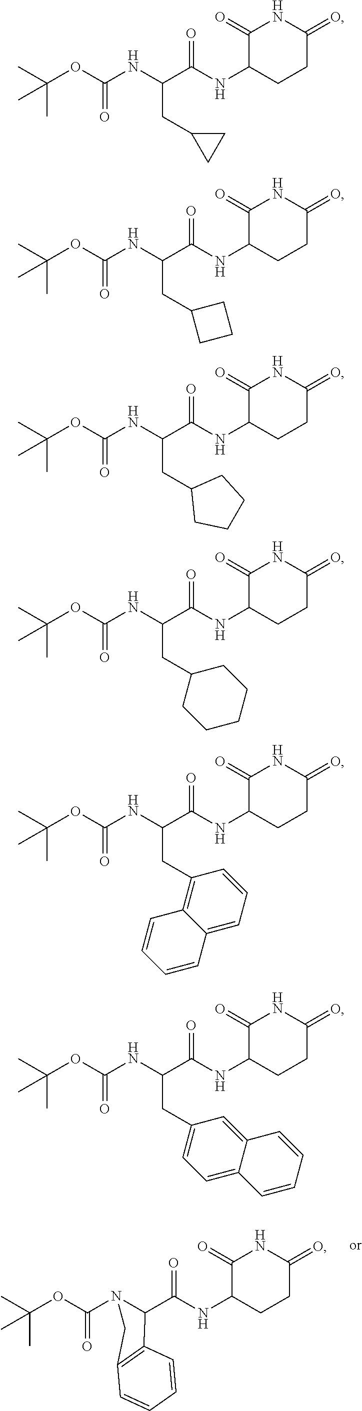

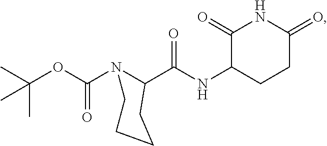 [SMILES: CC(C)(C)OC(=O)N1CCCCC1C(=O)NC1CCC(=O)NC1=O]

or a pharmaceutically acceptable salt or tautomer thereof.

### Compositions, Kits, and Combinations

The present disclosure provides pharmaceutical compositions comprising a compound disclosed herein (e.g., a compound of Formula (I′) or (I)), or a pharmaceutically acceptable salt or tautomer thereof, and optionally a pharmaceutically acceptable excipient. In certain embodiments, the pharmaceutical composition described herein comprises a compound of Formula (I′) or (I), or a pharmaceutically acceptable salt thereof, and a pharmaceutically acceptable excipient.

In certain embodiments, the compound of Formula (I′) or (I) is provided in an effective amount in the pharmaceutical composition. In certain embodiments, the effective amount is a therapeutically effective amount. In certain embodiments, the effective amount is a prophylactically effective amount.

In certain embodiments, the effective amount is an amount effective for treating a disease or disorder. In some embodiments, the disease or disorder is an inflammatory disease, proliferative disease, autoimmune disease, hematological disease, genetic disease, neurological disease, painful condition, metabolic disorder, infectious disease, cardiovascular disease, cerebrovascular disease, tissue repair disorder, pulmonary disease, dermatological disease, bone disease, or hormonal disease. In certain embodiments, the effective amount is an amount effective for treating a proliferative disorder in a subject in need thereof. In certain embodiments, the effective amount is an amount effective for treating cancer in a subject in need thereof. In certain embodiments, the effective amount is an amount effective for treating lung cancer, blood cancer, breast cancer, prostate cancer, pancreatic cancer, colorectal cancer, thyroid cancer, ovarian cancer, neuroblastoma, a carcinoma, a sarcoma, a melanoma, or a tumor. In certain embodiments, the effective amount is an amount effective for treating a hemopoietic cancer. In certain embodiments, the effective amount is an amount effective for treating a leukemia, a lymphoma, or multiple myeloma. In certain embodiments, the effective amount is an amount effective for treating nuclear protein of the testis (NUT) midline carcinoma, treatment-refractory acute myeloid leukemia, acute myeloid leukemia (AML), hairy cell leukemia (HCL), acute lymphocytic leukemia (ALL), acute promyelocytic leukemia (APL), chronic lymphocytic leukemia (CLL), chronic myeloid leukemia (CML), myeloproliferative neoplasms (MPN), systemic mastocytosis, plasmacytoma, multiple myeloma, myelodysplastic syndrome, triple negative breast cancer, estrogen receptor-positive breast cancer, small cell lung cancer, non-small cell lung cancer, castration resistant prostate cancer, pancreatic ductal adenocarcinoma, N-Myc Proto-Oncogene Protein (MYCN)-driven solid tumors, Ewing sarcoma, anaplastic thyroid carcinoma (ATC), medulloblastoma, or uveal melanoma. In certain embodiments, the effective amount is an amount effective for treating acute myeloid leukemia. In certain embodiments, the effective amount is an amount effective for treating multiple myeloma. In certain embodiments, the effective amount is an amount effective for treating del(5q) myelodysplastic syndrome. In certain embodiments, the effective amount is an amount effective for treating a cancer provided in the Definitions section.

In certain embodiments, the effective amount is an amount effective for treating an inflammatory disease. In certain embodiments, the effective amount is an amount effective for treating erythema nodosum leprosum, HIV-associated ulcers, and tuberculous meningitis. In certain embodiments, the effective amount is an amount effective for treating Crohn's disease, colitis, arthritis, rheumatoid arthritis, or inflammatory bowel disease. In certain embodiments, the effective amount is an amount effective for treating an inflammatory disease provided in the Definitions section.

In certain embodiments, the effective amount is an amount effective for treating an autoimmune disease. In certain embodiments, the effective amount is an amount effective for treating pulmonary fibrosis or systemic lupus erythematosus (SLE). In certain embodiments, the effective amount is an amount effective for treating Crohn's disease, colitis, arthritis, rheumatoid arthritis, or inflammatory bowel disease. In certain embodiments, the effective amount is an amount effective for treating an autoimmune disease provided in the Definitions section.

In certain embodiments, the effective amount is an amount effective for preventing a proliferative disorder in a subject in need thereof. In certain embodiments, the effective amount is an amount effective for preventing cancer in a subject in need thereof. In certain embodiments, the effective amount is an amount effective for preventing lung cancer, blood cancer, breast cancer, prostate cancer, pancreatic cancer, colorectal cancer, thyroid cancer, ovarian cancer, neuroblastoma, a carcinoma, a sarcoma, a melanoma, or a tumor. In certain embodiments, the effective amount is an amount effective for preventing a hemopoietic cancer. In certain embodiments, the effective amount is an amount effective for preventing a leukemia, a lymphoma, or multiple myeloma. In certain embodiments, the effective amount is an amount effective for preventing nuclear protein of the testis (NUT) midline carcinoma, treatment-refractory acute myeloid leukemia, acute myeloid leukemia (AML), hairy cell leukemia (HCL), acute lymphocytic leukemia (ALL), acute promyelocytic leukemia (APL), chronic lymphocytic leukemia (CLL), chronic myeloid leukemia (CML), myeloproliferative neoplasms (MPN), systemic mastocytosis, plasmacytoma, multiple myeloma, myelodysplastic syndrome, triple negative breast cancer, estrogen receptor-positive breast cancer, small cell lung cancer, non-small cell lung cancer, castration resistant prostate cancer, pancreatic ductal adenocarcinoma, N-Myc Proto-Oncogene Protein (MYCN)-driven solid tumors, Ewing sarcoma, anaplastic thyroid carcinoma (ATC), medulloblastoma, or uveal melanoma. In certain embodiments, the effective amount is an amount effective for preventing acute myeloid leukemia. In certain embodiments, the effective amount is an amount effective for preventing multiple myeloma. In certain embodiments, the effective amount is an amount effective for preventing del(5q) myelodysplastic syndrome. In certain embodiments, the effective amount is an amount effective for preventing a cancer provided in the Definitions section.

In certain embodiments, the effective amount is an amount effective for preventing an inflammatory disease. In certain embodiments, the effective amount is an amount effective for preventing erythema nodosum leprosum, HIV-associated ulcers, and tuberculous meningitis. In certain embodiments, the effective amount is an amount effective for preventing Crohn's disease, colitis, arthritis, rheumatoid arthritis, or inflammatory bowel disease. In certain embodiments, the effective amount is an amount effective for preventing an inflammatory disease provided in the Definitions section.

In certain embodiments, the effective amount is an amount effective for preventing an autoimmune disease. In certain embodiments, the effective amount is an amount effective for preventing pulmonary fibrosis or systemic lupus erythematosus (SLE). In certain embodiments, the effective amount is an amount effective for preventing Crohn's disease, colitis, arthritis, rheumatoid arthritis, or inflammatory bowel disease. In certain embodiments, the effective amount is an amount effective for preventing an autoimmune disease provided in the Definitions section.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of a target (i.e., the target to which B binds). In certain embodiments, the effective amount is an amount effective for promoting the degradation of a target (i.e., the target to which B binds) by a range between a percentage described in this paragraph and another percentage described in this paragraph, inclusive. In some embodiments, the degradation is the amount degraded in a cell. In some embodiments, the degradation is the amount degraded in a subject.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of a bromodomain-containing protein (e.g., BRD4). In certain embodiments, the effective amount is an amount effective for promoting the degradation of a bromodomain-containing protein (e.g., BRD4) by a range between a percentage described in this paragraph and another percentage described in this paragraph, inclusive. In some embodiments, the degradation is the amount degraded in a cell. In some embodiments, the degradation is the amount degraded in a subject.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of a bromodomain (e.g., BRD4). In certain embodiments, the effective amount is an amount effective for promoting the degradation of a bromodomain (e.g., BRD4) by a range between a percentage described in this paragraph and another percentage described in this paragraph, inclusive. In some embodiments, the degradation is the amount degraded in a cell. In some embodiments, the degradation is the amount degraded in a subject.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of a FKBP. In certain embodiments, the effective amount is an amount effective for promoting the degradation of a FKBP by a range between a percentage described in this paragraph and another percentage described in this paragraph, inclusive. In some embodiments, the degradation is the amount degraded in a cell. In some embodiments, the degradation is the amount degraded in a subject.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of FKBP12. In certain embodiments, the effective amount is an amount effective for promoting the degradation of FKBP12 by a range between a percentage described in this paragraph and another percentage described in this paragraph, inclusive. In some embodiments, the degradation is the amount degraded in a cell. In some embodiments, the degradation is the amount degraded in a subject.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of a cyclin dependent kinase. In certain embodiments, the effective amount is an amount effective for promoting the degradation of a cyclin dependent kinase by a range between a percentage described in this paragraph and another percentage described in this paragraph, inclusive. In some embodiments, the degradation is the amount degraded in a cell. In some embodiments, the degradation is the amount degraded in a subject.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of CDK4. In certain embodiments, the effective amount is an amount effective for promoting the degradation of CDK4 by a range between a percentage described in this paragraph and another percentage described in this paragraph, inclusive. In some embodiments, the degradation is the amount degraded in a cell. In some embodiments, the degradation is the amount degraded in a subject.

In certain embodiments, the effective amount is an amount effective for promoting the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of CDK6. In certain embodiments, the effective amount is an amount effective for promoting the degradation of CDK6 by a range between a percentage described in this paragraph and another percentage described in this paragraph, inclusive. In some embodiments, the degradation is the amount degraded in a cell. In some embodiments, the degradation is the amount degraded in a subject.

The present disclosure provides pharmaceutical compositions comprising a compound for use in treating or preventing a disease in a subject in need thereof. In some embodiments, the present disclosure provides pharmaceutical compositions comprising a compound that interacts with a protein for use in treating or preventing a disease in a subject in need thereof. In some embodiments, the present disclosure provides pharmaceutical compositions comprising a compound that interacts with a bromodomain-containing protein (e.g., BRD4), a bromodomain (e.g., BRD4), a kinase, a cyclin dependent kinase (e.g., CDK4, CDK6), or a FKBP (e.g., FKBP12) for use in treating or preventing a disease in a subject in need thereof. In some embodiments, the present disclosure provides pharmaceutical compositions comprising a compound that interacts with a bromodomain-containing protein (e.g., BRD4), a bromodomain (e.g., BRD4), a cyclin dependent kinase (e.g., CDK4, CDK6), or a FKBP (e.g., FKBP12) for use in treating or preventing a disease in a subject in need thereof.

In some embodiments, the present disclosure provides pharmaceutical compositions comprising a compound that interacts with a bromodomain-containing protein (e.g., BRD4), a bromodomain (e.g., BRD4), or a FKBP (e.g., FKBP12) for use in treating or preventing a disease in a subject in need thereof. In some embodiments, the present disclosure provides pharmaceutical compositions comprising a compound that interacts with a bromodomain-containing protein (e.g., BRD4), a bromodomain (e.g., BRD4), a kinase, a cyclin dependent kinase (e.g., CDK4, CDK6), or a FKBP (e.g., FKBP12) for use in treating or preventing a disease in a subject in need thereof. In some embodiments, the present disclosure provides pharmaceutical compositions comprising a compound that interacts with a bromodomain-containing protein (e.g., BRD4), a bromodomain (e.g., BRD4), a cyclin dependent kinase (e.g., CDK4, CDK6), or a FKBP (e.g., FKBP12) for use in treating or preventing a disease in a subject in need thereof.

The present disclosure provides pharmaceutical compositions for use in treating a proliferative disease in a subject in need thereof. In certain embodiments, the composition is for use in treating cancer. In certain embodiments, the composition is for use in treating lung cancer, blood cancer, breast cancer, prostate cancer, pancreatic cancer, colorectal cancer, thyroid cancer, ovarian cancer, neuroblastoma, a carcinoma, a sarcoma, a melanoma, or a tumor. In certain embodiments, the composition is for use in treating a hemopoietic cancer. In certain embodiments, the composition is for use in treating a leukemia, a lymphoma, or multiple myeloma. In certain embodiments, the composition is for use in treating nuclear protein of the testis (NUT) midline carcinoma, treatment-refractory acute myeloid leukemia, acute myeloid leukemia (AML), hairy cell leukemia (HCL), acute lymphocytic leukemia (ALL), acute promyelocytic leukemia (APL), chronic lymphocytic leukemia (CLL), chronic myeloid leukemia (CML), myeloproliferative neoplasms (MPN), systemic mastocytosis, plasmacytoma, multiple myeloma, myelodysplastic syndrome, triple negative breast cancer, estrogen receptor-positive breast cancer, small cell lung cancer, non-small cell lung cancer, castration resistant prostate cancer, pancreatic ductal adenocarcinoma, N-Myc Proto-Oncogene Protein (MYCN)-driven solid tumors, Ewing sarcoma, anaplastic thyroid carcinoma (ATC), medulloblastoma, or uveal melanoma. In certain embodiments, the composition is for use in treating acute myeloid leukemia. In certain embodiments, the composition is for use in treating multiple myeloma. In certain embodiments, the composition is for use in treating del(5q) myelodysplastic syndrome. In certain embodiments, the composition is for use in treating a cancer provided in the Definitions section.

The present disclosure provides pharmaceutical compositions for use in treating an inflammatory disease in a subject in need thereof. In certain embodiments, the composition is for use in treating erythema nodosum leprosum, HIV-associated ulcers, and tuberculous meningitis. In certain embodiments, the composition is for use in treating Crohn's disease, colitis, arthritis, rheumatoid arthritis, or inflammatory bowel disease. In certain embodiments, the composition is for use in treating an inflammatory disease provided in the Definitions section.

The present disclosure provides pharmaceutical compositions for use in treating an autoimmune disease in a subject in need thereof. In certain embodiments, the composition is for use in treating pulmonary fibrosis or systemic lupus erythematosus (SLE). In certain embodiments, the composition is for use in treating Crohn's disease, colitis, arthritis, rheumatoid arthritis, or inflammatory bowel disease. In certain embodiments, the composition is for use in treating an autoimmune disease provided in the Definitions section.

The present disclosure provides pharmaceutical compositions for use in preventing a proliferative disease in a subject in need thereof. In certain embodiments, the composition is for use in preventing cancer. In certain embodiments, the composition is for use in preventing lung cancer, blood cancer, breast cancer, prostate cancer, pancreatic cancer, colorectal cancer, thyroid cancer, ovarian cancer, neuroblastoma, a carcinoma, a sarcoma, a melanoma, or a tumor. In certain embodiments, the composition is for use in preventing a hemopoietic cancer. In certain embodiments, the composition is for use in preventing a leukemia, a lymphoma, or multiple myeloma. In certain embodiments, the composition is for use in preventing nuclear protein of the testis (NUT) midline carcinoma, treatment-refractory acute myeloid leukemia, acute myeloid leukemia (AML), hairy cell leukemia (HCL), acute lymphocytic leukemia (ALL), acute promyelocytic leukemia (APL), chronic lymphocytic leukemia (CLL), chronic myeloid leukemia (CML), myeloproliferative neoplasms (MPN), systemic mastocytosis, plasmacytoma, multiple myeloma, myelodysplastic syndrome, triple negative breast cancer, estrogen receptor-positive breast cancer, small cell lung cancer, non-small cell lung cancer, castration resistant prostate cancer, pancreatic ductal adenocarcinoma, N-Myc Proto-Oncogene Protein (MYCN)-driven solid tumors, Ewing sarcoma, anaplastic thyroid carcinoma (ATC), medulloblastoma, or uveal melanoma. In certain embodiments, the composition is for use in preventing acute myeloid leukemia. In certain embodiments, the composition is for use in preventing multiple myeloma. In certain embodiments, the composition is for use in preventing del(5q) myelodysplastic syndrome. In certain embodiments, the composition is for use in preventing a cancer provided in the Definitions section.

The present disclosure provides pharmaceutical compositions for use in preventing an inflammatory disease in a subject in need thereof. In certain embodiments, the composition is for use in preventing erythema nodosum leprosum, HIV-associated ulcers, and tuberculous meningitis. In certain embodiments, the composition is for use in preventing Crohn's disease, colitis, arthritis, rheumatoid arthritis, or inflammatory bowel disease. In certain embodiments, the composition is for use in preventing an inflammatory disease provided in the Definitions section.

The present disclosure provides pharmaceutical compositions for use in preventing an autoimmune disease in a subject in need thereof. In certain embodiments, the composition is for use in preventing pulmonary fibrosis or systemic lupus erythematosus (SLE). In certain embodiments, the composition is for use in preventing Crohn's disease, colitis, arthritis, rheumatoid arthritis, or inflammatory bowel disease. In certain embodiments, the composition is for use in preventing an autoimmune disease provided in the Definitions section.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve their ability to cross the blood-brain barrier, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., cancer). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage, the pharmaceutical compositions of this disclosure can be administered to humans and other animals orally, parenterally, intracisternally, intraperitoneally, topically, bucally, or the like, depending on the disease or condition being treated.

In certain embodiments, a pharmaceutical composition comprising a compound of Formula (I′) or (I) is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a compound of Formula (I′) or (I) into association with an excipient and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients, such as cocoa butter and suppository waxes, coloring agents, coating agents, sweetening, flavoring, and perfuming agents, may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the disclosure are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable excipient such as a lotion, cream, ointment, or soap. Useful excipients are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this disclosure. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the excipient for a topical formulation can be in the form of a hydroalcoholic system (e.g., quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical excipients include anhydrous solids and semisolids (such as gels and sticks); and aqueous based mousse systems.

Also encompassed by the disclosure are kits (e.g., pharmaceutical packs). The kits provided may comprise a pharmaceutical composition or compound (e.g., a compound of Formula (I′) or (I)) described herein and instructions for using the compound or composition. The kits provided may comprise a pharmaceutical composition or compound (e.g., a compound of Formula (I′) or (I)) described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating or preventing a disease (e.g., a proliferative disease, an inflammatory disease, or an autoimmune disease) in a subject in need thereof. In certain embodiments, the kits are useful for treating a proliferative disorder (e.g., a cancer) in a subject in need thereof. In certain embodiments, the kits are useful for treating cancer (e.g., a hematological cancer) in a subject in need thereof. In certain embodiments, the kits are useful for preventing cancer (e.g., a hematological cancer) in a subject in need thereof. In certain embodiments, the kits are useful for reducing the risk of developing cancer (e.g., a hematological cancer) in a subject in need thereof. In certain embodiments, the kits are useful for treating an inflammatory disease in a subject in need thereof. In certain embodiments, the kits are useful for preventing an inflammatory disease in a subject in need thereof. In certain embodiments, the kits are useful for reducing the risk of developing an inflammatory disease in a subject in need thereof. In certain embodiments, the kits are useful for treating an autoimmune disease in a subject in need thereof. In certain embodiments, the kits are useful for preventing an autoimmune disease in a subject in need thereof. In certain embodiments, the kits are useful for reducing the risk of developing an autoimmune disease in a subject in need thereof. In certain embodiments, the kits are useful for promoting the degradation of a bromodomain containing protein (e.g., BRD4), a bromodomain (e.g., BRD4), or a FKBP (e.g., FKBP12) in a subject or cell. In certain embodiments, the kits are useful for promoting the degradation of a bromodomain containing protein (e.g., BRD4), a bromodomain (e.g., BRD4), a kinase, a cyclin dependent kinase (e.g., CDK4, CDK6), or a FKBP (e.g., FKBP12) in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, a kit described herein may include one or more additional pharmaceutical agents described herein as a separate composition.

### Methods and Uses

Provided herein are methods and uses comprising a compound disclosed herein (e.g., a compound of Formula (I′) or (I)), or a pharmaceutically acceptable salt or tautomer thereof, or composition disclosed herein. The recited methods provided herein should be understood to encompass the corresponding embodiments and/or claim types (e.g., first medical use claims (e.g., compound/composition for use . . . , compound/composition for use as a medicament . . . ); purpose-limited composition claims (e.g., composition for use . . . ); second medical use/EPC2000 claims (e.g., use of a compound/composition for the treatment of . . . , or compound/composition for use in treating . . . ); and Swiss-type claims (e.g., use of compound/composition in the manufacture of a medicament for the treatment of . . . ).

Provided herein are methods of treating or preventing a disease in a subject, the method comprising administering to the subject a therapeutically effective amount of a compound disclosed herein (e.g., a compound of Formula (I′) or (I)), or a pharmaceutically acceptable salt or tautomer thereof, or composition disclosed herein. As noted above, for example, also provided herein are the corresponding uses including a compound disclosed herein (e.g., a compound of Formula (I′) or (I)), or a pharmaceutically acceptable salt or tautomer thereof, or composition disclosed herein for use in treating or preventing a disease.

In some embodiments, provided herein is a method of treating a disease associated with a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments the disease is a proliferative disease, an inflammatory disease, or an autoimmune disease.

In some embodiments, provided herein is a method of treating a disease associated with or mediated by a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments the disease is a proliferative disease, an inflammatory disease, or an autoimmune disease.

In some embodiments, provided herein is a method of treating a disease mediated by a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments the disease is a proliferative disease, an inflammatory disease, or an autoimmune disease. In some embodiments, the disease is cancer.

In some embodiments, provided herein is a method of treating a disease associated with aberrant (e.g., increased) activity of a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments the disease is a proliferative disease, an inflammatory disease, or an autoimmune disease.

In some embodiments, provided herein is a method of treating a disease mediated by aberrant (e.g., increased) activity of a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments the disease is a proliferative disease, an inflammatory disease, or an autoimmune disease.

In some embodiments, provided herein is a method of treating a disease associated with increased activity of a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments the disease is a proliferative disease, an inflammatory disease, or an autoimmune disease.

In some embodiments, provided herein is a method of treating a disease mediated by increased activity of a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the disease is cancer. In some embodiments the disease is a proliferative disease, an inflammatory disease, or an autoimmune disease.

In some embodiments, provided herein is a method of modulating (e.g., inhibiting) the activity of a target (i.e., the target that B binds to) in a subject, the method comprising administering to the subject an effective amount of a compound disclosed herein (e.g., a compound of Formula (I′) or (I)), or a pharmaceutically acceptable salt or tautomer thereof, or composition disclosed herein.

In some embodiments, provided herein is a method of inhibiting the activity of a target (i.e., the target that B binds to) in a subject, the method comprising administering to the subject an effective amount of a compound disclosed herein (e.g., a compound of Formula (I′) or (I)), or a pharmaceutically acceptable salt or tautomer thereof, or composition disclosed herein.

In some embodiments, provided herein is a method of modulating (e.g., inhibiting) the activity of a target (i.e., the target that B binds to) in a biological sample, the method comprising contacting the biological sample with an effective amount of a compound disclosed herein (e.g., a compound of Formula (I′) or (I)), or a pharmaceutically acceptable salt or tautomer thereof, or composition disclosed herein.

In some embodiments, provided herein is a method of inhibiting the activity of a target (i.e., the target that B binds to) in a biological sample, the method comprising contacting the biological sample with an effective amount of a compound disclosed herein (e.g., a compound of Formula (I′) or (I)), or a pharmaceutically acceptable salt or tautomer thereof, or composition disclosed herein.

In some embodiments, provided herein is a method of modulating (e.g., inhibiting) the expression of a gene that is regulated by a target (i.e., the target that B binds to) in a subject, the method comprising administering to the subject an effective amount of a compound disclosed herein (e.g., a compound of Formula (I′) or (I)), or a pharmaceutically acceptable salt or tautomer thereof, or composition disclosed herein.

In some embodiments, provided herein is a method decreasing the level of a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments the disease is a proliferative disease, an inflammatory disease, or an autoimmune disease. In some embodiments, the disease is cancer.

In some embodiments, provided herein is a method decreasing the concentration of a target (i.e., the target that B binds to) in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments the disease is a proliferative disease, an inflammatory disease, or an autoimmune disease. In some embodiments, the disease is cancer.

In some embodiments, provided herein is a method of inhibiting the expression of a gene that is regulated by a target (i.e., the target that B binds to) in a subject, the method comprising administering to the subject an effective amount of a compound disclosed herein (e.g., a compound of Formula (I′) or (I)), or a pharmaceutically acceptable salt or tautomer thereof, or composition disclosed herein.

In some embodiments, provided herein are methods of treating a disease associated with a bromodomain-containing protein, a bromodomain, a kinase, a cyclin dependent kinase, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, provided herein are methods of treating a disease associated with a bromodomain-containing protein, a bromodomain, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, provided herein are methods of treating a disease mediated by a bromodomain-containing protein, a bromodomain, a kinase, a cyclin dependent kinase, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, provided herein are methods of treating a disease mediated by a bromodomain-containing protein, a bromodomain, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the bromodomain-containing protein is BRD4. In some embodiments, the bromodomain is BRD4. In some embodiments, the method is selective for BRD4. In certain embodiments, the FKBP is FKBP12. In some embodiments, the method is selective for FKBP12. In some embodiments the cyclin dependent kinase is CDK4 or CDK6. In some embodiments the cyclin dependent kinase is CDK4. In some embodiments the cyclin dependent kinase is CDK6. In some embodiments, the method is selective for CDK6 or CDK4. In some embodiments, the method is selective for CDK6. In some embodiments, the method is selective for CDK4.

Also provided herein are methods of treating a disease associated with or mediated by a bromodomain-containing protein, a bromodomain, a kinase, a cyclin dependent kinase, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. Also provided herein are methods of treating a disease associated with or mediated by a bromodomain-containing protein, a bromodomain, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the bromodomain-containing protein is BRD4. In some embodiments, the bromodomain is BRD4. In some embodiments, the method is selective for BRD4. In certain embodiments, the FKBP is FKBP12. In some embodiments, the method is selective for FKBP12. In some embodiments the cyclin dependent kinase is CDK4 or CDK6. In some embodiments the cyclin dependent kinase is CDK4. In some embodiments the cyclin dependent kinase is CDK6. In some embodiments, the method is selective for CDK6 or CDK4. In some embodiments, the method is selective for CDK4. In some embodiments, the method is selective for CDK6.

In certain embodiments, provided herein are methods of treating a disease associated with aberrant activity a bromodomain-containing protein, a bromodomain, a kinase, a cyclin dependent kinase, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In certain embodiments, provided herein are methods of treating a disease associated with aberrant activity a bromodomain-containing protein, a bromodomain, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In certain embodiments, provided herein are methods of treating a disease mediated by aberrant activity a bromodomain-containing protein, a bromodomain, a kinase, a cyclin dependent kinase, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In certain embodiments, provided herein are methods of treating a disease associated with mediated by a bromodomain-containing protein, a bromodomain, or a FKBP in a subject in need thereof, the method comprising administering to the subject a therapeutically effective amount of a compound disclosed herein (e.g., a compound of Formula (I′) or (I)), or a pharmaceutically acceptable salt or tautomer thereof, or composition disclosed herein. In some embodiments, the aberrant activity is increased activity. In some embodiments, the bromodomain-containing protein is BRD4. In some embodiments, the bromodomain is BRD4. In some embodiments, the method is selective for BRD4. In certain embodiments, the FKBP is FKBP12. In some embodiments, the method is selective for FKBP12. In some embodiments the cyclin dependent kinase is CDK4 or CDK6. In some embodiments the cyclin dependent kinase is CDK4. In some embodiments the cyclin dependent kinase is CDK6. In some embodiments, the method is selective for CDK6 or CDK4. In some embodiments, the method is selective for CDK4. In some embodiments, the method is selective for CDK6.

The present disclosure provides methods for treating or preventing a disease or disorder. In some embodiments, the disease or disorder is an inflammatory disease, proliferative disease, autoimmune disease, hematological disease, genetic disease, neurological disease, painful condition, metabolic disorder, infectious disease, cardiovascular disease, cerebrovascular disease, tissue repair disorder, pulmonary disease, dermatological disease, bone disease, or hormonal disease.

The present disclosure provides methods for treating a proliferative disease. The present disclosure provides methods for preventing a proliferative disease. In some embodiments, the proliferative disease is cancer. In some embodiments, the cancer is lung cancer, blood cancer, breast cancer, prostate cancer, pancreatic cancer, colorectal cancer, thyroid cancer, ovarian cancer, neuroblastoma, a carcinoma, a sarcoma, a melanoma, or a tumor. In some embodiments, the cancer is a hemopoietic cancer. In some embodiments, the cancer is a leukemia, a lymphoma, or multiple myeloma. In some embodiments, the cancer is nuclear protein of the testis (NUT) midline carcinoma, treatment-refractory acute myeloid leukemia, acute myeloid leukemia (AML), hairy cell leukemia (HCL), acute lymphocytic leukemia (ALL), acute promyelocytic leukemia (APL), chronic lymphocytic leukemia (CLL), chronic myeloid leukemia (CML), myeloproliferative neoplasms (MPN), systemic mastocytosis, plasmacytoma, multiple myeloma, myelodysplastic syndrome, triple negative breast cancer, estrogen receptor-positive breast cancer, small cell lung cancer, non-small cell lung cancer, castration resistant prostate cancer, pancreatic ductal adenocarcinoma, N-Myc Proto-Oncogene Protein (MYCN)-driven solid tumors, Ewing sarcoma, anaplastic thyroid carcinoma (ATC), medulloblastoma, or uveal melanoma. In some embodiments, the cancer is acute myeloid leukemia. In certain embodiments, the cancer is multiple myeloma. In some embodiments, the myelodysplastic syndrome is del(5q) myelodysplastic syndrome. In some embodiments, the cancer is a cancer provided in the Definitions section.

The present disclosure provides methods for treating an inflammatory disease. The present disclosure provides methods for preventing an inflammatory disease. In certain embodiments, the inflammatory disease is selected from erythema nodosum leprosum, HIV-associated ulcers, and tuberculous meningitis. In some embodiments, the inflammatory disease is Crohn's disease, colitis, arthritis, rheumatoid arthritis, or inflammatory bowel disease. In some embodiments, the inflammatory disease is an inflammatory disease provided in the Definitions section.

The present disclosure provides methods for treating an autoimmune disease. The present disclosure provides methods for preventing an autoimmune disease. In some embodiments, the autoimmune disease is pulmonary fibrosis or systemic lupus erythematosus (SLE). In some embodiments, the autoimmune disease is Crohn's disease, colitis, arthritis, rheumatoid arthritis, or inflammatory bowel disease. In some embodiments, the autoimmune disease is an autoimmune disease provided in the Definitions section.

In some embodiments, the disease is associated with or mediated by a bromodomain, a bromodomain-containing protein, a histone methyltransferase, a kinase, a cytosolic signaling protein, a nuclear protein, a histone deacetylase, a lysine methyltransferase, a protein regulating angiogenesis, a protein regulating immune response, an aryl hydrocarbon receptor, a hormone receptor, or a transcription factor. In certain embodiments, the disease is associated with or mediated by bromodomain, kinase, or FKBP activity. In certain embodiments, the disease is associated with or mediated by bromodomain or FKBP activity. In some embodiments, the bromodomain-containing protein is BRD4. In some embodiments, the bromodomain is BRD4. In certain embodiments, the FKBP is FKBP12. In certain embodiments, the disease is associated with or mediated by kinase activity. In certain embodiments, the disease is associated with or mediated by cyclin dependent kinase activity. In some embodiments, the cyclin dependent kinase is CDK4 or CDK6. In some embodiments, the cyclin dependent kinase is CDK4. In some embodiments, the cyclin dependent kinase is CDK6.

Provided herein are methods of modulating the activity of a bromodomain-containing protein, a bromodomain, a kinase, or a FKBP in a subject, the method comprising administering to the subject an effective amount of a compound disclosed herein (e.g., a compound of Formula (I′) or (I)), or a pharmaceutically acceptable salt or tautomer thereof, or composition disclosed herein. Provided herein are methods of modulating the activity of a bromodomain-containing protein, a bromodomain, or a FKBP in a subject, the method comprising administering to the subject an effective amount of a compound disclosed herein (e.g., a compound of Formula (I′) or (I)), or a pharmaceutically acceptable salt or tautomer thereof, or composition disclosed herein. In some embodiments, provided herein is a method of modulating the activity of a bromodomain-containing protein, a bromodomain, a kinase, or a FKBP a cell, tissue, or biological sample, the method comprising contacting the cell, tissue, or biological sample with an effective amount of a compound disclosed herein (e.g., a compound of Formula (I′) or (I)), or a pharmaceutically acceptable salt or tautomer thereof, or composition disclosed herein. In some embodiments, provided herein is a method of modulating the activity of a bromodomain-containing protein, a bromodomain, or a FKBP a cell, tissue, or biological sample, the method comprising contacting the cell, tissue, or biological sample with an effective amount of a compound disclosed herein (e.g., a compound of Formula (I′) or (I)), or a pharmaceutically acceptable salt or tautomer thereof, or composition disclosed herein. Provided herein are methods of inhibiting the activity of a bromodomain-containing protein, a bromodomain, a kinase, or a FKBP in a subject, the method comprising administering to the subject an effective amount of a compound disclosed herein (e.g., a compound of Formula (I′) or (I)), or a pharmaceutically acceptable salt or tautomer thereof, or composition disclosed herein. Provided herein are methods of inhibiting the activity of a bromodomain-containing protein, a bromodomain, or a FKBP in a subject, the method comprising administering to the subject an effective amount of a compound disclosed herein (e.g., a compound of Formula (I′) or (I)), or a pharmaceutically acceptable salt or tautomer thereof, or composition disclosed herein. In some embodiments, provided herein is a method of inhibiting the activity of a bromodomain-containing protein, a bromodomain, a kinase, or a FKBP a cell, tissue, or biological sample, the method comprising contacting the cell, tissue, or biological sample with an effective amount of a compound disclosed herein (e.g., a compound of Formula (I′) or (I)), or a pharmaceutically acceptable salt or tautomer thereof, or composition disclosed herein. In some embodiments, provided herein is a method of inhibiting the activity of a bromodomain-containing protein, a bromodomain, or a FKBP a cell, tissue, or biological sample, the method comprising contacting the cell, tissue, or biological sample with an effective amount of a compound disclosed herein (e.g., a compound of Formula (I′) or (I)), or a pharmaceutically acceptable salt or tautomer thereof, or composition disclosed herein. In some embodiments, the bromodomain-containing protein is BRD4. In some embodiments, the bromodomain is BRD4. In certain embodiments, the FKBP is FKBP12. In certain embodiments, the kinase is a cyclin dependent kinase. In some embodiments, the cyclin dependent kinase is CDK4 or CDK6. In some embodiments, the cyclin dependent kinase is CDK6. In some embodiments, the cyclin dependent kinase is CDK4.

In certain embodiments, provided herein are methods of inhibiting the expression of a gene that is regulated by a bromodomain-containing protein, a bromodomain, a kinase, or a FKBP in a subject, the method comprising administering to the subject an effective amount of a compound disclosed herein (e.g., a compound of Formula (I′) or (I)), or a pharmaceutically acceptable salt or tautomer thereof, or composition disclosed herein. In certain embodiments, provided herein are methods of inhibiting the expression of a gene that is regulated by a bromodomain-containing protein, a bromodomain, or a FKBP in a subject, the method comprising administering to the subject an effective amount of a compound disclosed herein (e.g., a compound of Formula (I′) or (I)), or a pharmaceutically acceptable salt or tautomer thereof, or composition disclosed herein. In some embodiments, provided herein is a method of inhibiting the expression of a gene that is regulated by a bromodomain-containing protein, a bromodomain, a kinase, or a FKBP in a cell, tissue, or biological sample, the method comprising administering to the cell, tissue, or biological sample an effective amount of a compound disclosed herein (e.g., a compound of Formula (I′) or (I)), or a pharmaceutically acceptable salt or tautomer thereof, or composition disclosed herein. In some embodiments, provided herein is a method of inhibiting the expression of a gene that is regulated by a bromodomain-containing protein, a bromodomain, or a FKBP in a cell, tissue, or biological sample, the method comprising administering to the cell, tissue, or biological sample an effective amount of a compound disclosed herein (e.g., a compound of Formula (I′) or (I)), or a pharmaceutically acceptable salt or tautomer thereof, or composition disclosed herein. In some embodiments, the bromodomain-containing protein is BRD4. In some embodiments, the bromodomain is BRD4. In certain embodiments, the FKBP is FKBP12. In certain embodiments, the kinase is a cyclin dependent kinase. In some embodiments, the cyclin dependent kinase is CDK4 or CDK6. In some embodiments, the cyclin dependent kinase is CDK4. In some embodiments, the cyclin dependent kinase is CDK6.

In some embodiments, provided herein are methods of inducing the degradation of a protein in a subject, the method comprising administering to the subject an effective amount of a compound disclosed herein (e.g., a compound of Formula (I′) or (I)), or a pharmaceutically acceptable salt or tautomer thereof, or composition disclosed herein. In some embodiments, provided herein is a method of inducing the degradation of a protein in a cell, tissue, or biological sample, the method comprising administering to the cell, tissue, or biological sample an effective amount of a compound disclosed herein (e.g., a compound of Formula (I′) or (I)), or a pharmaceutically acceptable salt or tautomer thereof, or composition disclosed herein. In some embodiments, the protein is bromodomain-containing protein, a bromodomain, a kinase, or a FKBP. In some embodiments, the bromodomain-containing protein is BRD4. In some embodiments, the bromodomain is BRD4. In certain embodiments, the FKBP is FKBP12. In certain embodiments, the kinase is a cyclin dependent kinase. In some embodiments, the cyclin dependent kinase is CDK4 or CDK6. In some embodiments, the cyclin dependent kinase is CDK4. In some embodiments, the cyclin dependent kinase is CDK6.

In certain embodiments, the method is selective for bromodomain-containing protein, a bromodomain, a kinase, or a FKBP. In some embodiments, the method is selective for BRD4. In certain embodiments, the method is selective for FKBP12. In certain embodiments, the method is selective for CDK4. In certain embodiments, the method is selective for CDK6.

In some embodiments, the method is selective for promoting the degradation of bromodomain-containing protein, a bromodomain, a kinase, or a FKBP. In some embodiments, the method is selective for promoting the degradation of BRD4. In certain embodiments, the method is selective for promoting the degradation of FKBP12. In some embodiments, the method is selective for promoting the degradation of CDK4. In some embodiments, the method is selective for promoting the degradation of CDK6.

In some embodiments, the method inhibits IRF4 expression.

In certain embodiments, the method does not affect off-target transcription factors IKZF1, IKZF3, and SALL. In certain embodiments, the method does not affect off-target transcription factors IKZF1, IKZF3, and SALL4.

In some embodiments, the method mitigates off-target interactions compared to an immunomodulatory drug. In certain embodiments, the method mitigates off-target degradation compared to an immunomodulatory drug. In some embodiments, the method decreases side effects compared to an immunomodulatory drug. In certain embodiments, the immunomodulatory drug is selected from the group consisting of thalidomide, lenalidomide, and pomalidomide.

In certain embodiments, the methods of the disclosure comprise administering to the subject an effective amount of a compound of Formula (I′) or (I), or a pharmaceutically acceptable salt or tautomer thereof, or composition thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as the compound of Formula (I′) or (I), or at different times than the compound of Formula (I′) or (I). For example, the compound of Formula (I′) or (I) and any additional pharmaceutical agent(s) may be on the same dosing schedule or different dosing schedules. All or some doses of the compound of Formula (I′) or (I) may be administered before all or some doses of an additional pharmaceutical agent, after all or some does an additional pharmaceutical agent, within a dosing schedule of an additional pharmaceutical agent, or a combination thereof. The timing of administration of the compound of Formula (I′) or (I) and additional pharmaceutical agents may be different for different additional pharmaceutical agents.

In certain embodiments, the additional pharmaceutical agent comprises an agent useful in the treatment of proliferative disease. In certain embodiments, the additional pharmaceutical agent is useful in the treatment of cancer. In certain embodiments, the additional pharmaceutical agent is useful in the treatment of lung cancer, blood cancer, breast cancer, prostate cancer, pancreatic cancer, colorectal cancer, thyroid cancer, ovarian cancer, neuroblastoma, a carcinoma, a sarcoma, a melanoma, or a tumor. In certain embodiments, the additional pharmaceutical agent is useful in the treatment of a hematological cancer. In certain embodiments, the additional pharmaceutical agent cancer is useful in the treatment of multiple myeloma, a leukemia, or a lymphoma. In certain embodiments, the additional pharmaceutical agent cancer is useful in the treatment of a leukemia or a lymphoma. In certain embodiments, the additional pharmaceutical agent is useful in the treatment of nuclear protein of the testis (NUT) midline carcinoma, treatment-refractory acute myeloid leukemia, acute myeloid leukemia (AML), hairy cell leukemia (HCL), acute lymphocytic leukemia (ALL), acute promyelocytic leukemia (APL), chronic lymphocytic leukemia (CLL), chronic myeloid leukemia (CML), myeloproliferative neoplasms (MPN), systemic mastocytosis, plasmacytoma, multiple myeloma, myelodysplastic syndrome, triple negative breast cancer, estrogen receptor-positive breast cancer, small cell lung cancer, non-small cell lung cancer, castration resistant prostate cancer, pancreatic ductal adenocarcinoma, N-Myc Proto-Oncogene Protein (MYCN)-driven solid tumors, Ewing sarcoma, anaplastic thyroid carcinoma (ATC), medulloblastoma, or uveal melanoma. In certain embodiments, the additional pharmaceutical agent is useful in the treatment of acute myeloid leukemia. In certain embodiments, the additional pharmaceutical agent is useful in the treatment of multiple myeloma. In certain embodiments, the additional pharmaceutical agent is useful in the treatment of del(5q) myelodysplastic syndrome.

In certain embodiments, the additional pharmaceutical agent is useful in the treatment of an inflammatory disease. In certain embodiments, the additional pharmaceutical agent is useful in the treatment of erythema nodosum leprosum, HIV-associated ulcers, and tuberculous meningitis. n certain embodiments, the additional pharmaceutical agent is useful in the treatment of Crohn's disease, colitis, arthritis, rheumatoid arthritis, or inflammatory bowel disease.

In certain embodiments, the additional pharmaceutical agent is useful in the treatment of an autoimmune disease. In certain embodiments, the additional pharmaceutical agent is useful in the treatment of pulmonary fibrosis or systemic lupus erythematosus (SLE). In certain embodiments, the additional pharmaceutical agent is useful in the treatment of Crohn's disease, colitis, arthritis, rheumatoid arthritis, or inflammatory bowel disease.

Certain methods described herein may comprise administering one or more additional therapies in combination with the compounds described herein. In some embodiments, the method further comprises administering to the subject an additional therapy. In certain embodiments, the additional therapy is chemotherapy, radioimmunotherapy, surgical therapy, immunotherapy, radiation therapy, or targeted therapy, or any combination thereof.

### Examples

In order that the present disclosure may be more fully understood, the following examples are set forth. The synthetic and biological examples described in this application are offered to illustrate the compounds, pharmaceutical compositions, methods, and uses provided herein and are not to be construed in any way as limiting their scope.

### Materials and Methods

### General Supplies

(R, S)-Lenalidomide (BioVision 1862-25)
Pomalidomide (TCI P2074)
MLN4924 (Selleck Chemicals S7109)
3-(4,5-Dimethylthiazol-2-yl)-2,5-diphenyltetrazolium bromide (MTT) (Sigma-Aldrich 475989)
Mini bio-spin chromatography columns (Bio-Rad 732-6207)
B-PER (ThermoFisher 78248)
Lysozyme (VWR 97062-138)
Benzonase (Sigma-Aldrich E1014-5KU)
His-purification Dynabeads (Life Technologies 10103D)
Zeba 7 kDa desalting columns (0.5 mL and 5 mL, ThermoFisher 89882 and 89892)
Dulbecco's Modified Eagle's Medium (DMEM) (Genesee Scientific 25-500)
RPMI 1640 (Gibco 10-040-CV)
Trypsin-EDTA (Fisher Scientific 25200114)
Fetal bovine serum (FBS) (Peak Serum PS-FB2)
Penicillin-streptomycin (100×) (Lonza 17-602E)
NuPAGE 3-8% Tris-Acetate precast gels (ThermoFisher EA0375BOX)
Criterion XT Tris-Acetate precast gels (Bio-Rad 3450131)
4-15% Criterion TGX precast gels (Bio-Rad 5671085)
16.5% Mini-PROTEAN® Tris-Tricine gels (Bio-Rad 4563066)
iBlot 2 nitrocellulose transfer stack (Invitrogen IB23001; IB23002).
BCA solution (BCA Reagent A) (VWR 786-847)
Copper Solution (BCA Reagent B) (VWR 76825-860)
Vivaspin spin concentrators (Cytiva)
Opti-MEM I Reduced Serum Medium (ThermoFisher 31985070)
Lipofectamine 3000 Transfection Reagent (ThermoFisher L300001)
TransIT-Pro Transfection Reagent (Mirus MIR 5760)

### Cloning Reagents

Q5 site-directed mutagenesis kit (New England BioLabs E0552S)

### Immunoprecipitation

Non-denaturing cell lysis buffer (10×) (Cell Signaling Technology 9803S)
Protease/phosphatase inhibitor cocktail (100×) (Cell Signaling Technology 5872S)
Protein G magnetic beads (Cell Signaling Technology 70024)
Anti-FLAG M2 beads (Sigma-Aldrich M8823-1ML)
2× SDS-PAGE loading buffer (concentration for 1×: 50 mM Tris-HCl, 2% SDS, 10% glycerol, 1% β-mercaptoethanol, 0.02% bromophenol blue)
3× FLAG peptide (Sigma-Aldrich F4799-4MG)

### Global Quantitative Proteomics

S-Trap micro (Protifi)
Triethylammonium bicarbonate buffer (Sigma-Aldrich T7408-100ML)
Trypsin/Lys-C mix, mass spectrometry grade (Promega V5073)
Trypsin, sequencing grade modified (Promega V5111)
TMT10plex isobaric label reagent set (ThermoFisher 90406)
TMTpro 16plex label reagent set (ThermoFisher A44520)
Pierce high pH reversed-phase peptide fractionation kit (ThermoFisher 84868)

### Alphascreen

Optiplate 384 (Perkin Elmer 6007290)
TopSeal A (Perkin Elmer 6050185)
GST-BRD4 (Epicypher 15-0013)
His-CRBN/DDB1 (generous gift from Bristol Myers Squibb)
AlphaScreen Glutathione Donor beads (Perkin Elmer 6765301)
Nickel chelate AlphaLISA acceptor beads (Perkin Elmer AL108M)
Alphascreen Omnibeads (Perkin Elmer 6760626D)
GST-His₆ (EMD Millipore 12-523)

### NanoBRET

NanoBRET CRBN Ternary Complex Starter Kit (ND2720)
NanoBRET Nano-Glo Detection Systems (N1662)
MG132 (Selleck Chemicals S2619)

### In Vitro Ubiquitylation

Pierce IP lysis buffer (ThermoFisher 87788)
Protease inhibitor cocktail (Sigma-Aldrich 11873580001, 1 tablet dissolved in 2 mL water for a 25× stock solution)
Anti-FLAG M2 beads (Sigma-Aldrich M8823-1ML)
3× FLAG peptide (Sigma-Aldrich F4799-4MG)
UBE1 (E1, Boston Biochem E-305-025)
UbcH5a (E2, aka UBE2D1, Boston Biochem E2-616-100)
UbcH5c (E2, aka UBE2D3, Boston Biochem E2-627-100)
KO ubiquitin (Boston Biochem UM-NOK-01M)
Ubiquitin aldehyde (Boston Biochem U-201-050)
Mg-ATP (Boston Biochem B-20)
E3 ligase buffer (Boston Biochem B-71)
MG132 (Boston Biochem I13005M)
MG101 (Tocris 3358)

### Electroporation

Neon Transfection System 100 μL Kit (ThermoFisher MPK10025)
Neon Transfection Tubes (ThermoFisher MPT100)

### RT-qPCR

Monarch Total RNA Miniprep Kit (New England BioLabs T2010S)
Luna Universal One-step RT-qPCR Kit (New England BioLabs E3005)

### Synthetic Peptides

HBB[42-60]: FFESFGDLSTPDAVMGNPK (Biomatik) (SEQ ID NO: 44)
ACTB[96-113]: VAPEEHPVLLTEAPLNPK (Biomatik) (SEQ ID NO: 37)
HBA[63-91]: VADALTNAVAHVDDMPNALSALSDLHAHK (Biomatik) (SEQ ID NO: 40)

### Mammalian Cell Lines

HEK293T and MM.1S cells were obtained from American Type Culture Collection (ATCC). HEK293T cells stably expressing FLAG-CRBN (HEK-CRBN cells) and CRBN knockdown HEK293T cells were kindly provided by the Deshaies Lab (California Institute of Technology).¹⁰¹ Whole blood samples were obtained from Stanford Blood Center. Fresh bovine eyes were obtained from Nebraska Scientific (PZ7K486F).

### Bacterial Strains

Mach1 cells (ThermoFisher C862003)
5-alpha Competent E. coli (High Efficiency) (New England BioLabs C2987H)
BL21 (DE3) (New England Biolabs C2527H)
NEB Stable Competent E. coli (New England BioLabs C3040I)
Mix & Go! E. coli Transformation Buffer Set (Zymo Research T3002)

### Antibodies

TABLE E1

| | Antibody | Host | | | |
|---|---|---|---|---|---|
| No. | Name | Species | Dilution | Supplier | Catalog# |
| 1 | BRD4 | Rabbit | 1:1000 | Cell Signaling | 13440S |
| | (E2A7X) | mAb | | Technology | |
| 2 | Vinculin | Mouse | 1:1000 | Bio-Rad | MCA465 |
| | (V284) | mAb | | | GA |
| 3 | CRBN | Rabbit | 1:1000 | Sigma-Aldrich | HPA045910 |
| | | pAb | | | |
| 4 | FLAG (M2) | Mouse | 1:1000 | Sigma-Aldrich | F1804 |
| | | mAb | | | |
| 5 | FLAG | Rabbit | 1:1000 | Cell Signaling | 14793S |
| | | mAb | | Technology | |
| 6 | Ikaros/IKZF1 | Rabbit | 1:1000 | Cell Signaling | 9034S |
| | (D10E5) | mAb | | Technology | |
| 7 | Aiolos/IKZF3 | Rabbit | 1:1000 | Novus | NBP2- |
| | | pAb | | Biologicals | 16938 |
| 8 | β-actin (C4) | Mouse | 1:1000 | Santa Cruz | sc-47778 |
| | | mAb | | Biotechnology | |
| 9 | FKBP12 | Rabbit | 1:1000 | Abcam | ab24373 |
| | | pAb | | | |
| 10 | CDK6 | Rabbit | 1:1000 | Cell Signaling | D4S8S |
| | | mAb | | Technology | |
| 11 | GFP (D5.1) | Rabbit | 1:1000 | Cell Signaling | 2956S |
| | | mAb | | Technology | |
| 12 | Hemoglobin | Rabbit | 1:1000 | Abcam | ab231584 |
| | | pAb | | | |
| 13 | Anti-mouse- | Goat | 1:10,000 | Rockland | 610-1302 |
| | HRP | | | Immunochemicals | |
| 14 | Anti-rabbit- | Goat | 1:10,000 | Rockland | 611-1302 |
| | HRP | | | Immunochemicals | |
| 15 | Anti-mouse- | Goat | 1:10,000 | LI-COR | 925-32210 |
| | IRDye ® | | | Biosciences | |
| | 800CW | | | | |

### Plasmids

TABLE E2

| No. | Plasmid name | Source |
|---|---|---|
| 1 | pET29-eSRTa | Addgene 75144 |
| 2 | pET28a:GFP | Addgene 60733 |
| 3 | pET28a-GFP-LPETG-Hi_{S6} | This work |
| | (SEQ ID NO: 5) | |
| 4 | pGEX2T-FKBP12 | A gift from the Liu lab |
| | | (Harvard University) |
| 5 | pGEX2T-FKBP12-LPETG | This work |
| 6 | CRBN CRISPR/Cas9 KO (h) | SCBT sc-412142 |
| 7 | CRBN HDR (h) | SCBT sc-412142-HDR |

### Primers

TABLE E3

| No. | Primer name | Sequence (5′ to 3′) |
|---|---|---|
| 1 | GFP-LPETG-F | CACCACCACCACCACCAC (SEQ ID NO: |
| | (SEQ ID NO: | 45) |
| | 5) | |
| 2 | GFP-LPETG-R | ACCGGTTTCCGGGAGGCTACCACCGGTCTT |
| | (SEQ ID NO: | GTACAGCTCGTCCATGC (SEQ ID NO: |
| | 5) | 46) |
| 3 | GST-FKBP-F1 | GGCGGCAGCGGAGTGCAGGTGGAAACCAT |
| | | (SEQ ID NO: 47) |
| 4 | GST-FKBP-R1 | TTTTGGAGGATGGTCGCCAC (SEQ ID |
| | | NO: 48) |
| 5 | GST-FKBP-F2 | GGCCACCATCACCATCACCACTAAACTAGA |
| | | ATTCATCGTGACTGACTGA (SEQ ID |
| | | NO: 49) |
| 6 | GST-FKBP-R2 | GCCCGTCTCAGGTAACGAGCCTCCAGTCAG |
| | | ATCCTCTTCTGAGATGAGTT (SEQ ID |
| | | NO: 50) |
| 7 | β-actin-F | CATGTACGTTGCTATCCAGGC (SEQ ID |
| | | NO: 51) |
| 8 | β-actin-R | CTCCTTAATGTCACGCACGAT (SEQ ID |
| | | NO: 52) |
| 9 | BRD2-F | AATGGCACAAACGCTGGAAAA (SEQ ID |
| | | NO: 53) |
| 10 | BRD2-R | CACTGGTAACACTGCCCTG (SEQ ID |
| | | NO: 54) |
| 11 | BRD4-F | GAGCTACCCACAGAAGAAACC (SEQ ID |
| | | NO: 55) |
| 12 | BRD4-R | GAGTCGATGCTTGAGTTGTGTT (SEQ |
| | | ID NO: 56) |
| 13 | IKZF1-F | TTCCGTGATCCTTTTGAGTGC (SEQ ID |
| | | NO: 57) |
| 14 | IKZF1-R | CTCGCGTTATGTGCGACGA (SEQ ID |
| | | NO: 58) |

### Instrumentation

Protein quantification by bicinchoninic acid assay (BCA) was measured on a multi-mode microplate reader FilterMax F3 (Molecular Devices LLC, Sunnyvale, CA, 570 nm filter). AlphaScreen readings were performed on a SpectraMax i3x plate reader equipped with an AlphaScreen Detection Cartridge (384 STD) (Molecular Devices LLC, Sunnyvale, CA). Protein concentration and OD600 measurements were measured by Nanodrop OneC Microvolume UV-Vis Spectrophotometer (ThermoFisher). Cell lysis was performed using a Branson Ultrasonic Probe Sonicator (model 250). Fluorescence and chemiluminescence imaging was performed using an Azure Imager c600 or 600 (Azure Biosystems, Inc., Dublin, CA). Protein purification and analytical SEC was performed using an ÄKTA pure 25 equipped with a F9-R fraction collector, a C9n conductivity monitor, and computer running UNICORN v6.3.2.89 (GE Healthcare). All proteomics data were obtained on a Waters ACQUITY UPLC system connected in line to an Orbitrap Fusion Lumos Tribrid Mass Spectrometer (ThermoFisher) within the Mass Spectrometry and Proteomics Resource Laboratory at Harvard University. Intact protein mass spectra were collected using a Bruker Impact II q-TOF mass spectrometer coupled to an Agilent 1290 HPLC within the Mass Spectrometry and Proteomics Resource Laboratory at Harvard University. Western blotting transfer was performed using an Invitrogen iBlot 2 dry blotting system. RT-qPCR was performed using a iQ5 Multicolor Real-Time PCR Detection System (Bio-Rad).

Electroporation was performed using a Neon electroporation system (ThermoFisher). Flow cytometry was conducting using Fortessa and LSRII flow cytometers (both BD). Cell numbers and viability were measured using TC20 automated cell counter (Bio-Rad). Samples were dried using a Vacufuge Plus (Eppendorf).

### Software

Data was analyzed and visualized using Microsoft Excel (v16.44) and GraphPad Prism (v8.4.3). MOE (Chemical Computing Group, v2019.01) was used to model the protein complexes. DNA and protein sequences were analyzed using Geneious (v11.0.3). Proteomics data was analyzed using Proteome Discoverer (v2.4.1.15). Images were made using ImageJ (NIH, v1.52q), Adobe Photoshop (v21.1.1) and Adobe Illustrator (v24.1).

### General Procedures

### Cell Culture, Transfection Protocols, and Collection

Cells were cultured in DMEM or RPMI 1640 supplemented with 10% heat-inactivated fetal bovine serum (FBS) and 1× penicillin-streptomycin. Mouse Embryonic Fibroblast (MEF) cells were cultured in DMEM supplemented with 15% heat-inactivated FBS and 1× penicillin-streptomycin. Cells were grown at 37° C. in a humidified atmosphere with 5% CO₂. Mycoplasma testing was performed regularly for all cell lines to check for contamination.

For the collection of cell pellets, cells were dissociated and collected by two PBS washes and centrifugation at 500×g, 24° C., 3 min, followed by a final PBS wash of the pellets. The pellets were flash frozen with liquid nitrogen and stored at −80° C. until use.

### Western Blotting Procedures

Unless otherwise noted, cells were lysed by probe sonication (5 sec on, 3 sec off, 15 sec in total, 11% amplitude) in 1-2% SDS in 1×PBS and cleared by centrifugation at 21,000×g, 4° C., 10 min. As needed, a BCA assay was performed to determine the protein concentration of lysates and the concentration was adjusted using lysis buffer. 5× SDS-PAGE loading buffer (5% (v/v) β-mercaptoethanol, 0.02% (w/v) bromophenol blue, 30% (v/v) glycerol, 10% (w/v) SDS/250 mM Tris pH 6.8) was added to the protein samples to a final concentration of 1× and the samples were heated at 95° C. for 5 min. Protein samples (8-15 μL per lane) were loaded on NuPAGE 3-8% Tris-Acetate precast gels or Criterion XT Tris-Acetate precast gels for high molecular weight proteins, 6/12% Tris-Glycine gels or 4-15% Criterion™ TGX™ precast gels for medium molecular weight proteins, or 16.5% Mini-PROTEAN® Tris-Tricine gels for low molecular weight proteins. Gels were transferred to membranes using the Invitrogen iBlot 2 dry blotting system and iBlot 2 nitrocellulose transfer stacks, using program PO (1 min at 20 V, 4 min at 23 V, 2 min at 25 V) for most proteins and 8 min at 25 V for high molecular weight proteins. Membranes were stained with Ponceau S solution to visualize transfer and protein loading. After being blocked with 5% milk or BSA in TBST at 24° C. for 1 h, the membranes were incubated with primary antibodies at 24° C. for 1 h or at 4° C. for 1 to 24 h. Membranes were washed (3×5 min) with TBST and incubated with secondary antibodies at 24° C. for 1 h. Membranes were washed (3×5 min) with TBST and the results were obtained by chemiluminescence and/or IR imaging using Azure 600 or c600.

### Molecular Cloning Procedures

Plasmids were constructed using standard cloning procedures. Insertions and deletions were accomplished using the Q5 site-directed mutagenesis kit. Sequences were verified by Sanger sequencing (Quintara Bio) prior to use.

### Generation of CRBN Knockout HEK293T Cells by CRISPR Cas9

CRBN CRISPR/Cas9 knockout plasmids mix, consisting of three CRBN-specific gRNA and HDR plasmids, were purchased from Santa-Cruz Biotechnology. The HDR plasmid contains puromycin resistance and RFP encoding genes to be inserted into the DSB site. Transfection was performed using similar procedure as described by the manufacturer. WT HEK293T were seeded in 6-well plate with DMEM+10% FBS without antibiotics to reach 90% confluency at the time of transfection. For each well, 0.5 μg CRBN CRISPR/Cas9 KO plasmid and 0.5 μg HDR plasmid were diluted in 150 μL Opti-MEM I and 4 μL Lipofectamine 3000 was then added. This mix was added into another 150 μL Opti-MEM I containing 4 μL Lipofectamine 3000 and incubated for 20 min. The mixture was added dropwise into each well and incubated for 48 h. Selection was performed with 4 μg/mL puromycin for 7 days. The top 1% RFP-expressing population was the selected by sorting and the sorted cells were expanded and validated for CRBN knockout by Western blotting.

### BRD4 Degradation Assay

1.5×10⁶ HEK293T cells were seeded in DMEM supplemented with 10% FBS and 1× penicillin-streptomycin and incubated at 37° C., 5% CO₂ for 30 min, then treated with compounds of interest and incubated at 37° C., 5% CO₂ for 4 h prior to collection and lysis. If noted, cells were treated with 1 μM MLN4924 for 1 h before treatment with compounds following the initial 30 min incubation. Compounds were dissolved in DMSO, and the final DMSO concentration after addition of the compound to the cells did not exceed 0.2% v/v.

### FKBP12 Degradation Assay

1.5×10⁶ HEK293T cells were seeded in DMEM supplemented with 10% FBS and 1× penicillin-streptomycin and incubated at 37° C., 5% CO₂ for 30 min, then treated with compounds of interest and incubated at 37° C., 5% CO₂ for 18 h prior to collection and lysis. If noted, cells were treated with 1 μM MLN4924 for 1 h before treatment with compounds following the initial 30 min incubation. Compounds were dissolved in DMSO and the final DMSO concentration after addition of the compound to the cells did not exceed did not exceed 0.2% v/v.

### CDK6 Degradation Assay

1.5×10⁶ Jurkat cells were seeded in RPMI supplemented with 10% FBS and 1× penicillin-streptomycin and incubated at 37° C., 5% CO₂ for 30 min, then treated with compounds of interest and incubated at 37° C., 5% CO₂ for 24 h prior to collection and lysis. If noted, cells were treated with 1 μM MLN4924 for 1 h before treatment with compounds following the initial 30 min incubation. All compounds were dissolved in DMSO and the final DMSO concentration after addition of the compound to the cells did not exceed did not exceed 0.2% (v/v).

### Immunoprecipitation

For in vivo FLAG-tag co-immunoprecipitation, 5.0×10⁶ HEK-CRBN cells were seeded and incubated at 37° C., 5% CO₂ for 30 min, and then were treated with 1 μM MLN4924 for 1 h. Compounds of interest were added to a final concentration of 25 μM and the cells were incubated at 37° C., 5% CO₂ for 2 h. The cells were collected and lysed in 1× protease/phosphatase inhibitor/1× non-denaturing lysis buffer (500 μL) and clarified by centrifugation (21,000×g, 4° C., 10 min). The soluble portion of the lysate was collected and 200 μL of lysate was incubated with of protein G magnetic beads for 20 min (20 μL) to minimize the non-specific binding. The solution was collected and was then incubated with anti-FLAG M2 magnetic beads (40 μL) on a tube rotator at 4° C. for 1.5 h. The magnetic beads were washed with 1× non-denaturing lysis buffer (5×500 μL). Then, the enriched proteins were eluted by addition of 40 μL of 2× SDS-PAGE loading buffer and heated at 95 ºC for 5 min prior to Western blot analysis.

For in vitro FLAG-tag co-immunoprecipitation, 5.0×10⁶ HEK-CRBN cells were collected by centrifugation, lysed in 1× protease/phosphatase inhibitor/1× non-denaturing lysis buffer (400 μL), and cleared by centrifugation (21,000×g, 4° C., 10 min). The soluble portion of the lysate was collected and 200 μL of lysate was incubated with compound of interest at 4° C. for 2 h. Then, the mixture was added to pre-washed anti-FLAG M2 magnetic beads (40 μL) and incubated on a tube rotator at 4° C. for 1.5 h. The magnetic beads were washed with 1× non-denaturing lysis buffer (5×500 μL). The enriched proteins were eluted by the addition of 40 μL of 2× SDS-PAGE loading buffer and heated at 95° C. for 5 min prior to Western blot analysis.

### AlphaScreen

AlphaScreen buffer (3× stock solution: 150 mM HEPES pH 7.4, 600 mM NaCl, 0.3% w/v BSA, 3 mM TCEP) was prepared fresh for each experiment. A 3× stock solution of each compound (60 μM, 3% DMSO final/1× AlphaScreen buffer) and a series of 2-fold serial dilutions, in 3% DMSO/1× AlphaScreen buffer, were prepared fresh for each experiment. A solution of 750 nM His₆-CRBN/DDB1, 375 nM GST-BRD4/1× AlphaScreen buffer (5 μL) was added to each well of a 384-well Optiplate. Then, compound (5 μL) was added, with each concentration assayed in triplicate. As a positive control, 10 μL 100 nM GST-His₆ was added to three wells. The plate was sealed with TopSeal A, centrifuged (200× g, 25° C., 1 min), and incubated at 25° C. for 1 h. Under low light, the seal was removed and 5 μL of 60 μg/mL AlphaScreen Glutathione Donor beads, 60 μg/mL AlphaLISA Nickel Chelate Donor beads/1× AlphaScreen buffer was added to each well. The plate was sealed with TopSeal A, centrifuged (200× g, 25° C., 1 min), and incubated at 25° C. for 1 h. The seal was removed and the plate was analyzed. Prior to analysis, the plate reader was calibrated with a plate containing 15 μL of 20 μg/mL Omnibeads/1× AlphaScreen buffer in the corner wells. Analysis was performed in Graphpad Prism, using the vehicle-treated wells as the baseline, fitting the signal from each compound to a Gaussian curve, and calculating the area under the curve (AUC). AUC results from each plate were normalized to dBET6.

### NanoBRET Assay

4×10⁶ HEK293T cells were seeded in a 100 mm TC-treated dish in 10 mL DMEM supplemented with 10% FBS. Cells were incubated 18-24 h (37° C., 5% CO₂). The transfection mix, containing 12 μg HaloTag-CRBN fusion vector, 0.12 μg NanoLuc-BRD4 FL fusion vector, 1.2 mL Opti-MEM I reduced serum media without phenol red, and 12 μL TransIT Pro, was incubated at 25° C. for 15 min prior to dropwise addition to the culture. Cells were incubated 20 h (37° C., 5% CO₂). Cells were trypsinized and washed, then resuspended at 2×10⁵ cells/mL in Opti-MEM I reduced serum media without phenol red. For ligand samples, 5.5 μL DMSO was added per 5.5 mL cell suspension. For +ligand samples, 5.5 μL of 0.1 mM HaloTag NanoBRET 618 Ligand was added per 5.5 mL cell suspension. In an opaque white TC-treated 96 well plate, cells were reseeded, with 100 μL cell suspension per well. Cells were incubated 18-24 h (37° C., 5% CO₂). From a 50 mM stock in DMSO, MG132 was diluted to 50 μM in Opti-MEM I reduced serum media without phenol red and 25 μL was added to each well. Cells were incubated 30 min (37° C., 5% CO₂). From 1 mM stocks in DMSO, compounds being analyzed were diluted to 6 μM in Opti-MEM I reduced serum media without phenol red and 25 μL was added to each well, with each compound dosed in triplicate in cells±ligand. Cells were incubated 2 h (37° C., 5% CO₂). Nanoglo substrate was diluted to 4×, from a 500× stock, in Opti-MEM I reduced serum media without phenol red, and 50 μL was added to each well. Within 10 min, luminescence at 450 nm and 618 nm (15 nm bandpass filters, 1 s integration time) was read on an SpectraMax i3x plate reader. BRET ratios were calculated as the luminescence at 618 nm divided by the luminescence at 450 nm, and corrected BRET ratios were calculated by subtracting the BRET ratio −ligand from the BRET ratio +ligand for each compound.

### Computational Modeling

Computational models were generated in MOE version 2020.09. The IMiD ligand from the indicated crystal structure was adapted to the indicated dipeptide and the adapted complex was subjected to energy minimization with Amber10:EHT force field followed by preparation with Protonate 3D.

### Global Quantitative Proteomics Sample Preparation

Global proteomics samples were prepared in biological triplicate for each condition. 1.5×10⁶ HEK293T or MM.1S cells were seeded in 6-well plates and incubated at 37° C., 5% CO₂ for 30 min. Small molecules of interested were then added, and cells were incubated at 37° C., 5% CO₂ for the indicated time. Cells were collected according to the described general procedure, lysed by probe sonication (5 sec on, 3 sec off, 15 sec in total, 11% amplitude) in lysis buffer (5% SDS in 50 mM triethylammonium bicarbonate (TEAB), pH 7.55), and cleared by centrifugation (21,000× g, 4° C., 10 min). After protein quantification by BCA protein assay, the lysates were diluted to 1 mg/mL with the lysis buffer. The diluted lysates (100 μL) were reduced by addition of dithiothreitol (20 mM) at 24° C. for 30 min then alkylated by addition of iodoacetamide (40 mM) and incubation in the dark at 24° C. for 30 min.

The samples were desalted and digested using a S-Trap micro.^{102,103} In brief, samples were acidified by the addition of phosphoric acid to a final concentration of 1.2%. S-Trap buffer (90% methanol, 0.1 M TEAB, pH 7.1, 900 μL) was then added. Each sample was transferred to a S-Trap micro column. Using a vacuum manifold, the columns were washed with S-Trap buffer (3×150 μL). To digest the S-trap-bound proteins, 4 μg of trypsin/Lys-C mix resuspended in 40 μL 50 mM TEAB pH 8.0 was added to each column and incubated at 37° C. for 16 h without rotation. The digested peptides were eluted by sequential addition of 50 mM TEAB pH 8.0 (40 μL), ddH₂O (40 μL) and 0.2% formic acid, 50% acetonitrile/water (35 μL), with each elution collected by centrifugation (4,000× g, 24° C., 1 min) in a clean Eppendorf tube. The eluted samples were concentrated to dryness in a vacufuge and resuspended in 45 or 80 μL ddH₂O for 9 or 16 samples, respectively. For each resuspended sample, 5 μL was taken for labeling with TMT reagent (2 μL) at 24° C. for 1 h such that the combined total protein was 100 μg. Hydroxylamine (50%, 1.2 μL) was added to each sample to quench the TMT reagent, and the samples were incubated at 24° C. for 15 min. The TMT-labeled samples were combined and dried in a vacufuge. The dried sample was resuspended in 300 μL 0.1% trifluoroacetic acid (TFA) and fractionated to 20 fractions using a Pierce high pH reversed-phase peptide fractionation kit. The peptides were eluted sequentially by 4% acetonitrile/0.1% triethylamine (TEA) through 20% acetonitrile/0.1% TEA in 1% acetonitrile increments (17 fractions), followed by 25%, 30% and 50% acetonitrile/0.1% TEA. The first fraction (4% acetonitrile/0.1% TEA) was excluded from LC-MS/MS analysis. The other fractions were concentrated to dryness and each sample was resuspended in 20 μL of 0.1% formic acid prior to LC-MS/MS analysis.

### Proteomics Mass Spectrometry Acquisition Procedures

Desalted and fractionated samples were resuspended in 0.1% formic acid/water (20 μL per sample). The sample (2.0 μL) was loaded onto a C18 trap column (3 cm, 3 μm particle size C10 Dr. Maisch 150 μm I.D) and then separated on an analytical column (Thermo Scientific Acclaim PepMap 100, 2 μm particle size, 250 mm length, 75 μm internal diameter) at 150 nL/min with a Thermo Scientific Ultimate 3000 system connected in line to a Thermo Scientific Orbitrap Fusion Tribrid. The column temperature was maintained at 50° C. Peptides were eluted using a multi-step gradient at a flow rate of 0.15 μL/min over 120 min (0-5 min, 2-5% acetonitrile in 0.1% formic acid/water; 5-95 min, 5-50%; 95-105 min, 50-98%; 105-115 min, 98%; 115-116 min, 98-2%; 116-120 min, 2%). The electrospray ionization voltage was set to 2 kV and the capillary temperature was set to 275° C. Dynamic exclusion was enabled with a repeat count of 2, repeat duration of 30 sec, exclusion list size of 400, and exclusion duration of 30 sec. MS1 scans were performed over 400-2000 m/z at resolution 120,000. HCD fragmentation was performed on the top ten most abundant precursors exhibiting a charge state from two to five at a resolving power setting of 50,000 and fragmentation energy of 37% in the Orbitrap. CID fragmentation was applied with 35% collision energy, and resulting fragments were detected using the normal scan rate in the ion trap.

### Proteomics Mass Spectrometry Acquisition Procedures for Global Proteomics

Desalted and fractionated samples were resuspended in 0.1% formic acid/water (20 μL per sample). The sample (10 μL) was loaded onto a C18 trap column (3 cm, 3 μm particle size C18 Dr. Maisch 150 μm I.D) and then separated on an analytical column (50 cm PharmaFluidics, Belgium) at 0.2 μL/min with a Thermo Scientific Ultimate 3000 system connected in line to a Thermo Scientific Orbitrap Fusion Lumos Tribrid. The column oven temperature was maintained at 35° C. Peptides were eluted using a multi-step gradient at a flow rate of 0.2 μL/min over 90 min (0-15 min, 7% acetonitrile in 0.1% formic acid/water; 15-65 min, 7-37%; 65-75 min, 37-95%; 75-85 min, 95%; 85-90 min, 95-2%). The electrospray ionization voltage was set to 2.2 kV and the capillary temperature was set to 275° C. Dynamic exclusion was enabled with a mass tolerance of 10 ppm and exclusion duration of 150 sec. MS1 scans were performed over 410-1800 m/z at resolution 120,000. HCD fragmentation was performed on the top ten most abundant precursors exhibiting charge states from two to five at a resolving power setting of 60,000 and fragmentation energy of 38% in the HCD Orbitrap. CID fragmentation was applied with 35% collision energy, and resulting fragments were detected using the normal scan rate in the ion trap.

### Mass Spectrometry Data Analysis

Analysis was performed in Thermo Scientific Proteome Discoverer version 2.4.1.15. The raw data were searched against SwissProt human (Homo sapiens) protein database (19 Aug. 2016; 20,156 total entries) and contaminant proteins using the Sequest HT algorithm. Searches were performed with the following guidelines: spectra with a signal-to-noise ratio greater than 1.5; mass tolerance of 10 ppm for the precursor ions and 0.02 Da (HCD) and 0.6 Da (CID) for fragment ions; full trypsin digestion; 2 missed cleavages; variable oxidation on methionine residues (+15.995 Da); static carboxyamidomethylation of cysteine residues (+57.021 Da); static TMT labeling (+226.163 Da for TMT-10plex or +304.207 Da for TMTpro-16plex) at lysine residues and N-termini. The TMT reporter ions were quantified using the Reporter Ions Quantifier node and normalized such that the summed peptide intensity per channel was equal. Peptide spectral matches (PSMs) were filtered using a 1% false discovery rate (FDR) using Percolator. PSMs were filtered to PSMs in only one protein group with an isolation interference under 70%. For the obtained proteome, the data were further filtered to include only master proteins with high protein FDR confidence and exclude all contaminant proteins. For the proteomics of MM.1S cells, the data were additionally filtered to proteins with greater than or equal to 2 unique peptides. For the p-value and fold change calculations, the data were further processed according to the methods of Huber and coworkers.¹⁰⁴ The model incorporates dependence of the variance on the mean intensity and a variance-stabilizing data transformation. In brief, missing abundances were filled in with minimum noise level computed by taking the minimum for each channel in Control and minimum for each channel in Treatment. A set of 2000 centroids were generated at random from the absolute maximum in the Control and Treatment and the absolute minimum in Control and Treatment, and a minimum noise level was generated using a K-means clustering method. If one abundance was missing, then the instance was filled with the geometric mean of the PSM for Control or Treatment. If all abundances were missing for Control and Treatment or the variance between existing abundances was above 30%, the PSM was removed. P-values for the abundance ratios were calculated using the t-test (background) method.

### Identification of C-terminal Cyclic Imide Modification Sites

Data were analyzed as above with the following modifications. For the analysis of global proteomics datasets obtained from CPTAC, the RAW datasets were processed according to the methods in the respective publication for fully tryptic peptides and spectra that did not receive a confident peptide spectral match were searched for semi-tryptic sequences at the C-terminus and an additional dynamic modification of dehydration on asparagine or glutamine residues (−18.015 Da). For the proteomics of red blood cells, data were searched against fragments ions with semi-trypsic digestion and an additional dynamic modification of dehydration on asparagine or glutamine residues (−18.015 Da). The data were filtered with a 5% or 1% false discovery rate (FDR) using Percolator, respectively.

### Intact Protein Mass Spectrometry

The column used was an Agilent PLRP-S (50 mm length, 5 μm particle size, 4.6 mm ID, 1000 Å pore size). The column was maintained at 70° C. during the run. For each sample, 10 μL protein solution was either injected directly through a union and eluted with 0.1% formic acid/60% acetonitrile/water or injected onto the column and eluted using the following method with mobile phases A (0.1% formic acid/water) and B (0.1% formic acid/acetonitrile). Prior to the gradient, the column was maintained at 0% B for 2 min to wash salts. Then, a linear gradient was applied over 10 min to a final concentration of 100% B. The column was maintained at 100% B for 1 min to wash before changing to 0% B over 0.1 min. Then, the column was re-equilibrated at 0% B for 5.9 min prior to the next run. The data was internally calibrated using sodium formate clusters injected at the end of each run. The data was analyzed using Bruker Compass DataAnalysis (v. 4.3), and deconvoluted using the maximum entropy algorithm between selected mass ranges (27-29 kDa for GFP or 25-40 kDa for GST-FKBP12). Modification masses used included: GFP fluorophore formation=−20.0256 Da, dehydration=−18.0153 Da, methylation=+14.0266 Da.

### Overexpression and Purification of SrtA

The procedure for overexpression and purification of SrtA was adapted from Liu and co-workers and Zenobi-Wong and co-workers.^{105,106} BL21 (DE3) cells transformed with pET29-eSrtA (Plasmid 1) were used to inoculate overnight cultures of LB+50 μg/mL kanamycin, which were then incubated at 37° C. with shaking at 200 rpm for approximately 16 h. For each large-scale overexpression, kanamycin was added to 750 mL autoclaved LB to a final concentration of 50 μg/mL and the culture was inoculated with overnight culture diluted 1:100. The overexpression cultures were incubated at 37° C. with shaking at 200 rpm until the OD600 was approximately 0.5-0.8, at which point IPTG was added to a final concentration of 0.1 mM and the temperature was reduced to 30° C. The cultures were incubated for 3 h prior to collecting the cells by centrifugation, flash freezing with liquid nitrogen, and storing at −80° C.

Up to 2 pellets, each from a 750 mL overexpression, were purified simultaneously. To purify protein, cell pellets were thawed on ice or in cool water, then resuspended in 10 mL of 0.1 mg/mL lysozyme, 1:1000 benzonase, 6 mM MgCl₂/B-PER per pellet. Lysates were incubated at 25° C. with shaking for 15 min, then were clarified by centrifugation (15,000×g, 4° C., 10 min), and syringe filtration (0.45 μm). The His-tagged protein was crudely purified on a Ni-bound 1 mL HiTrap Chelating HP column using standard methods, equilibrating and washing with 25 mM imidazole/PBS and eluting with a gradient to 500 mM imidazole/PBS. Protein-containing fractions, as determined by A280, not eluting with the dead volume were concentrated to approximately 1 mL and further purified on a S75 10/300 GL column, pre-equilibrated and run with TBS. Protein-containing fractions not eluting with the dead volume were collected and concentrated to approximately 100 μM with a 10 kDa MWCO spin concentrator, with protein concentration determined by measuring the A280 (ε=14,440 M⁻¹ cm⁻¹). The protein was aliquoted, flash frozen with liquid nitrogen, and stored at −80° C.

### Overexpression and Purification of GFP-LPETG (SEQ ID) NO: 5)

The protocol for overexpression and purification of GFP-LPETG (SEQ ID NO: 5) was adapted from Liu and coworkers.¹⁰⁷ pET28a-GFP-LPETG (SEQ ID NO: 5) (Plasmid 3) was constructed by adding the sequence encoding TGGSLPETG-His₆ (SEQ ID NO: 59) to the C-terminus of GFP in pET28a:GFP (Plasmid 2) using primers 1 and 2. BL21 (DE3) cells transformed with pET28a-GFP-LPETG (SEQ ID NO: 5) and were used to inoculate overnight cultures of LB+50 μg/mL kanamycin, which were then incubated at 37° C. with shaking at 200 rpm for approximately 16 h. For each large-scale overexpression, kanamycin was added to 750 mL autoclaved LB to a final concentration of 50 μg/mL and the large-scale overexpression cultures were inoculated with overnight culture diluted 1:100. The overexpression cultures were incubated at 37° C. with shaking at 200 rpm until the OD600 was approximately 1, at which point IPTG was added to a final concentration of 0.45 mM and the temperature was reduced to 20° C. The cultures were incubated for approximately 16 h prior to collecting the cells by centrifugation, flash freezing with liquid nitrogen, and storing at −80° C.

Up to 3 pellets, each from a 750 mL overexpression, were purified simultaneously. To purify protein, cell pellets were thawed on ice or in cool water, then resuspended in 8.3 mL of 25 mM imidazole, 1× protease inhibitor, 1% Triton-X 100/PBS per pellet. Lysates were combined and sonicated (30 sec on, 10 sec off, 5 min total, 25% amplitude) on ice. Lysates were clarified by centrifugation (20,000× g, 4° C., 10 min) and syringe filtration (0.45 μm). The His-tagged protein was crudely purified on a Ni-bound 1 mL HiTrap Chelating HP column using standard methods, equilibrating and washing with 25 mM imidazole/PBS and eluting with a gradient to 500 mM imidazole/PBS. Protein-containing fractions, as determined by A280, were concentrated to approximately 1 mL and further purified on a S75 10/300 GL column, pre-equilibrated and run with TBS. Protein-containing fractions not eluting with the dead volume were collected and concentrated to approximately 200 μM with a 10 kDa MWCO spin filter, then diluted to 50 μM with TBS, with protein concentration determined by measuring the A488 (8=55,000 M⁻¹ cm⁻¹). The protein was aliquoted, flash frozen with liquid nitrogen, and stored at −80° C.

### Overexpression and Purification of GST-FKBP12-LPETG (SEQ ID) NO: 5)

pGEX2T-FKPB12-LPETG (SEQ ID NO: 5) (Plasmid 5) was constructed from pGEX2T-FKBP12 (Plasmid 4) using primers 3 and 4 to remove the internal His₆ tag and primers 5 and 6 to add the C-terminal LPETG-His₆ (SEQ ID NO: 5) tag. Overexpression in Rossetta 2 (DE3) cells was performed as previously described for pGEX2T-FKBP12.108

Up to 3 pellets, each from a 750 mL overexpression, were purified simultaneously. To purify protein, cell pellets were thawed on ice or in cool water, then resuspended in 8.3 mL of 25 mM imidazole, 1 mM PMSF, 1% Triton-X 100/PBS per pellet. Lysates were combined and sonicated (30 sec on, 10 sec off, 5 min total, 25% amplitude) on ice together. Lysates were clarified by centrifugation (20,000× g, 4° C., 10 min) and syringe filtration (0.45 μm). The His-tagged protein was crudely purified on a Ni-bound 1 mL HiTrap Chelating HP column using standard methods, equilibrating and washing with 25 mM imidazole/PBS and eluting with a gradient to 500 mM imidazole/PBS. Protein-containing fractions, as determined by A280, were concentrated to approximately 1 mL and further purified on a S75 10/300 GL column, pre-equilibrated and run with TBS. Protein-containing fractions not eluting with the dead volume were collected and concentrated to approximately 500 μM with a 10 kDa MWCO spin filter, with protein concentration determined by measuring the A280 (€=53,080 M⁻¹ cm⁻¹). The protein was aliquoted, flash frozen with liquid nitrogen, and stored at −80° C.

### Sortase Reaction

Conditions for the sortase reaction were adapted from Liu and coworkers and Ploegh and coworkers. 107.109 For each reaction, 10 μL of 50 μM GFP-LPETG (SEQ ID NO: 5) or 100 μM GST-FKBP12-LPETG (SEQ ID NO: 5) was combined with 2 μL of 10 mM peptide substrate in 37 μM of 100 mM Tris pH 7.5, 150 mM NaCl, 5 mM CaCl₂). Then, 1 μL of 97 μM eSrtA was added and the reaction was mixed by flicking. The reaction was allowed to incubate at 25° C. for 1 h. Non-reacted GFP-LPETG (SEQ ID NO: 5) and eSrtA was removed by adding 25 μL of washed His-purification Dynabeads and incubating with inversion for 15 min. The supernatant was collected using a magnetic tube rack. To remove excess peptide substrate, a 0.5 mL Zeba 7 kDa desalting column was equilibrated three times with 300 μL of experiment buffer (centrifuging 1,500×g, 25° C., 1 min for each equilibration). The sample was added to the equilibrated column, which was centrifuged (1500×g, 25° C., 2 min). The flowthrough was collected. The final concentration of GFP was determined by measuring the A488 (€=55,000 M⁻¹ cm⁻¹).¹¹⁰ For in cellulo experiments, the reaction was performed on a larger scale, with the volume of Dynabeads reduced to be equal to the volume of 50 μM GFP-LPETG (SEQ ID NO: 5) or 100 μM GST-FKBP12-LPETG (SEQ ID NO: 5) used. The larger-scale reactions were desalted using a 5 mL Zeba 7 kDa spin desalting column then concentrated using a 3 kDa MWCO spin concentrator. If not used immediately, proteins were flash frozen with liquid nitrogen and stored at −80° C. until use.

### In Vitro Ubiquitylation

5.0×10⁶ HEK-CRBN cells were collected per pellet, flash frozen with liquid nitrogen, and stored at −80° C. until use. Approximately 1 pellet per 1.5 samples was thawed on ice, and each pellet was resuspended in 1× protease inhibitor cocktail/Pierce IP lysis buffer (250 μL). Lysates were incubated on ice for 10 min, then were clarified by centrifugation (21,000×g, 4° C., 10 min). The soluble portions of the lysates were collected, with 1 mL lysate per tube, and 150 μL of TBS-washed anti-FLAG M2 beads were added. Samples were incubated at 4° C. on a roller for 1 h. Using a magnetic tube rack, the beads were collected and washed 3× with 1 mL TBS. Each sample was then eluted with by adding 100 μL of 100 ng/μL 3× FLAG peptide/TBS and incubating at 4° C. on a roller for 1 h. The eluent was collected. Substrate proteins were diluted to 5-7.5 μM in PBS, using the same protein concentration for all samples in each experiment, and 25× stocks of small molecules were prepared in 2.5% DMSO/PBS. Ubiquitylation mastermixes were prepared at 2× with and without E1 and E2 enzymes. Final concentrations (1×) of the mastermix components were 0.2 μM UBE1, 2 μM UbcH5a, 1 mM UbcH5c, 400 μg/mL KO ubiquitin, 1 μM ubiquitin aldehyde, 1× Mg-ATP, 1× E3 ligase buffer, 10 μM MG132, 100 nM MG101. Reactions were prepared by combining 6.25 μL FLAG eluent, 5.25 μL of target protein (6.25 μL if no small molecule competition was performed in experiment), 1 μL of 25× small molecule stock in 2.5% DMSO/PBS, and 12.5 μL of ubiquitylation mastermix in PCR tubes. Reactions were incubated at 30° C. for 90 min then were stopped by the addition of 6.25 μL of 5× SDS-PAGE loading buffer. Samples were heated at 95° C. for 5 min prior to analysis by SDS-PAGE and Western blotting.

### Cellular Degradation of C-Terminally-Tagged Proteins

HEK293T cells were grown to 80-90% confluency in DMEM+10% FBS without antibiotics (DMEM+/−). Cells were detached by trypsinization and washed with PBS, then resuspended in PBS and counted. For each sample, an equal number of cells (7×10⁵-2.5×10⁶) were aliquoted into a 1.7 mL tube and pelleted. Electroporation mixes were prepared for each sample type by combining 311 μL Neon buffer R with 3.6 μL DMSO or 100× compound or, for experiments without small molecule competition, 315 μL Neon buffer R and 45 μL of 50-60 μM protein in PBS, using the same protein concentration for all samples in each experiment, or PBS for mock samples. Immediately prior to each electroporation, the PBS was removed from the pelleted cells and the pellet was resuspended in 110 μL of electroporation mix. The sample was taken up into a 100 μL tip attached to a Neon pipette, and the pipette tip was submerged in a Neon cuvette containing 3 mL Neon buffer E2. The sample was then electroporated (800 V, 25 msec, 2 pulses). The cells were then dispensed into 1 mL warmed PBS. This process was repeated for each sample, with each tip used for 3 electroporation cycles. Cells were then pelleted by centrifugation and the supernatant was removed. Cells were resuspended in 0.5 mL trypsin-EDTA solution and incubated at 37° C. for 5 min. Trypsinization was quenched by the addition of 0.5 mL DMEM+/− and cells were again pelleted. The supernatant was removed. Each sample was resuspended in 1 mL DMEM+/− and transferred to a well of a TC-treated 12-well plate, with DMSO or 1000× compound stock added to the media. The samples were then incubated at 37° C., 5% CO₂ until 6 h after electroporation. Media was removed by aspiration and the cells were washed with PBS. Cells were detached by trypsinization, collected by centrifugation, and washed with PBS. For analysis of GFP levels, each sample was resuspended in 500 μL PBS with 10 μL 0.5 mg/mL propidium iodide added to allow for exclusion of dead cells. Cells were analyzed by flow cytometry (mCherry and FITC on LSRII or dsRed and FITC on Fortessa). At least 9,600 events were analyzed for each sample. Relative GFP level was determined by subtracting the geometric mean GFP signal among live cells in the mock sample from the geometric mean GFP signal among live cells for each sample, then normalizing the resulting values to the value for GFP-His₆. For FKBP12 samples, protein solutions were prepared in TBS instead of PBS, and following electroporation and trypsinization cells were dispensed into DMEM+/− in an untreated 12-well plate. Cells were collected by centrifugation 6 h after electroporation and were lysed in 1% SDS, 1× protease inhibitor/PBS by brief electroporation (5 sec, 10% amplitude). Protein concentration was normalized by BCA assay, and samples were analyzed by Western blotting.

### Alternative Cellular Degradation of C-Terminally-Tagged Proteins

Cells were grown to 80-90% confluency in FBS-supplemented media without antibiotics (+/−) prior to electroporation. Cells were detached by trypsinization as necessary and washed with PBS, then resuspended in PBS and counted. For each sample, an equal number of cells (7×10⁵-2.5×10⁶) were aliquoted into a 1.7 mL tube and pelleted. Electroporation mixes were prepared for each sample type. Electroporation mixes for 100 μL tips were prepared by combining 311 μL Neon buffer R with 3.6 μL DMSO or 100× compound or, for experiments without small molecule competition, 315 μL Neon buffer R and 45 μL of 50-60 μM protein in PBS or TBS, using the same protein concentration for all samples in each experiment, or buffer alone for control samples. Electroporation mixes for 10 μL tips were prepared for each sample type by combining 43.25 μL Neon buffer R with 0.5 μL DMSO or 100× compound and 6.25 μL of 50 μM protein in PBS, or TBS for control samples. Immediately prior to each electroporation, the PBS was removed from the pelleted cells and the pellet was resuspended in 110 μL or 12 μL of electroporation mix (for 100 and 10 μL tips, respectively). The sample was taken up into a tip attached to a Neon pipette, and the pipette tip was submerged in a Neon cuvette containing 3 mL Neon buffer E2. The sample was then electroporated (HEK 293T cells 800 V, 25 msec, 2 pulses; Jurkat cells 1325 V, 10 msec, 3 pulses; MEF cells 1350 V, 30 msec, 1 pulse). The cells were then dispensed into 10 volumes of warmed PBS. This process was repeated for each sample, with each tip used for 3 electroporation cycles. Cells were then pelleted by centrifugation and the supernatant was removed. Cells were resuspended in 0.5 mL or 50 μL trypsin-EDTA solution (for 100 and 10 μL tips, respectively) and incubated at 37° C. for 5 min. Trypsinization was quenched by the addition of 0.5 mL DMEM+/− with DMSO or 1000× compound stock added to the media and cells electroporated with 100 μL tips were again pelleted. The supernatant was removed. Each sample was resuspended in 1 mL DMEM+/− and transferred to a well of a 12-well plate, with DMSO or 1000× compound stock added to the media. Cells electroporated with 10 μL tips were transferred directly to a 24-well plate. The samples were then incubated at 37° C., 5% CO₂ until 6 h after electroporation. Cells were detached by trypsinization, agitation, and/or scraping, collected by centrifugation, and washed with PBS.

For analysis of GFP levels, each sample was resuspended in 500 μL PBS with 50 nM SYTOX Blue or 10 μL 0.5 mg/mL propidium iodide added to allow for exclusion of dead cells. Cells were analyzed by flow cytometry (mCherry, Pacific Blue, and FITC on LSRII or dsRed and FITC on Fortessa). At least 9,600 events were analyzed for each sample. Relative GFP level was determined by subtracting the arithmetic mean GFP signal among live cells in the control sample from the arithmetic mean GFP signal among live cells for each sample, then normalizing the resulting values to the value for GFP-His₆ or GFP-Me. For FKBP12 samples, cells were collected by centrifugation 6 h after electroporation and were lysed in 1% SDS, 1× protease inhibitor/PBS by brief electroporation (5 sec, 10% amplitude). Protein concentration was normalized by BCA assay, and samples were analyzed by Western blotting.

### Time-Course Study of Hydrolysis of GFP-FcQ FeN

The indicated proteins were diluted to 1 μM in PBS (120 μL), with each condition prepared in triplicate. Samples were incubated in a sand bath pre-heated at 37° C., and 20 μL was taken from each sample at t=0 h (immediately after sample preparation), 24 h, 48 h, 72 h, and 96 h. Samples were flash-frozen in liquid nitrogen and stored at −80° C. until analysis by intact protein MS. The maximum peak intensity within 2 Da of the expected masses of the cyclized and hydrolyzed forms of each protein was used to determine the % cQ or cN remaining relative to the starting proportion.

### Preparation of Red Blood Cells

Whole blood (˜10 mL) was pelleted by centrifugation (500×g) at 24° C. for 10 min. The plasma supernatant was aspirated and the cell pellet was washed with 150 mM NaCl in PBS, pH 7.4 (1×5 mL) and PBS (2×5 mL). The washed red blood cells were then resuspended in PBS (5 mL), aliquoted, flash frozen with liquid nitrogen, and stored at −80° C. until use.

### Global Proteomics of Red Blood Cells

Red blood cells (50 μL) were resuspended in 5% SDS in 50 mM triethylammonium bicarbonate (TEAB), pH 7.55 (400 μL) and clarified by centrifugation (21,000× g, 4° C., 10 min). An equivalent suspension of red blood cells (50 μL) was prepared in 400 μL non-SDS lysis buffer (25 mM Tris-HCl pH 7.4, 150 mM NaCl, 1 mM EDTA, 1% NP-40 and 5% glycerol) for protein concentration measurement. The concentration of lysate was measured by NanoDrop using the “Oxy-hemoglobin custom method”.¹¹¹ As both conditions gave homogenous lysates and SDS led to the degradation of heme,¹¹² the protein concentration of cells in 5% SDS in TEAB was determined by that of the equivalent suspension. The lysate was diluted to 1 mg/mL and 100 μL of the lysate was digested on an S-trap micro column as previously described. Following TMT-labeling, the dried TMT-labeled sample was resuspended in 300 μL 0.1% TFA and fractionated to 6 fractions using the Pierce high pH reversed-phase peptide fractionation kit at 5%, 10%, 15%, 20%, 35% and 50% acetonitrile/0.1% TEA. The fractions were concentrated to dryness and each sample was resuspended in 20 μL of 0.1% formic acid prior to LC-MS/MS analysis.

### Preparation of Bovine Lens Lysates

Lenses were extracted from fresh whole bovine eyes and rinsed with PBS prior to flash-freezing with liquid nitrogen. Lenses were stored at −80° C. Each frozen lens was then ground to a powder using a mortar and pestle cooled with liquid nitrogen. The frozen powder was stored at −80° C. To prepare lysate, 100 mg crushed lens powder was suspended in 5% SDS/50 mM TEAB pH 7.4. The sample was lysed by sonication (5 s on, 2 s off, 10 s total, 10% amplitude) and clarified by centrifugation (21,000× g, 4° C., 10 min). Protein concentration was measured by BCA assay.

### Proteomics for Cyclic Imide Detection in Red Blood Cells and Bovine Lens

Red blood cells and bovine lens samples were prepared in biological triplicate and quadruplicate for each condition, respectively. The lysates were diluted to 1 mg/mL and 100 μL of the lysates were loaded on an S-trap micro column similarly as “Global Quantitative Proteomics Sample Preparation”. To digest the S-trap-bound proteins, 2 μL g of trypsin resuspended in 40 μL 50 mM TEAB pH 7.4 was added to each column and incubated at 47° C. for 1 h without rotation. The eluted samples were concentrated to dryness in a vacufuge and resuspended in 25 μL ddH₂O. For each resuspended sample, 10 μL was taken for labeling with TMT reagent (10 μL) at 24° C. for 1 h. TMT labeling was quenched by 1M Tris-C1 (5 μL, pH 7.6) instead of hydroxylamine to minimize the hydrolysis of cyclic imides. For base ablation of the cyclic imides, the dried, digested samples were incubated with 1% triethylamine/H₂O (100 μL, pH 12.0) at 65° C. for 30 min. The base-treated samples were concentrated to dryness before TMT labeling. The combined sample after TMT labeling was resuspended in 900 μL 0.1% TFA and 300 μL was taken to fractionation into 6 fractions at 5%, 10%, 15%, 20%, 25%, 35% and 50% acetonitrile/0.1% TEA using the Pierce high pH reversed-phase peptide fractionation kit. The first fraction (5% acetonitrile/0.1% TEA) was excluded from LC-MS/MS analysis. Immediately after the elution, each fraction was acidified by the addition of 5 μL of 10% formic acid. The fractions were concentrated to dryness and each sample was resuspended in 20 μL of 0.1% formic acid prior to LC-MS/MS analysis. Samples for cyclic imide detection were dried in a vacufuge set at 24° C., and the dried samples were stored at −20° C. or −80° C. to avoid long exposure to higher temperature.

### LC-MS Analysis of HBB [42-60] Peptide

A synthetic peptide corresponding to hemoglobin beta residues 42-60 (HBB[42-60]) was purchased from Biomatik. Peptide solution (20 μL, 10 μg/mL stock in ddH2O) was taken to labeling with TMT-10plex reagent (10 μL) at 24° C. for 1 h. TMT labeling was quenched by hydroxylamine (6 μL, 50%) at 24° C. for 15 min. The sample was dried in a vacufuge and resuspended in 0.1% formic acid prior to LC-MS/MS analysis. Approximately 10 ng of peptide was injected on a Thermo Orbitrap Fusion Lumos Tribrid. The peptide was eluted using a multistep gradient at a flow rate of 0.2 μL/min over 90 min (0-5 min, 2% acetonitrile in 0.1% formic acid/water; 5-7 min, 2-5%; 7-50 min, 5-45%; 50-80 min, 45-95%; 80-90 min, 95%). The electrospray ionization voltage was set to 2 kV and the capillary temperature was set to 275° C. MSI scans were performed over 410-1400 m/z at resolution 120,000. HCD fragmentation was performed on the top ten most abundant precursors exhibiting a charge state from two to five at a resolving power setting of 60,000 and fragmentation energy of 37% in the Orbitrap. CID fragmentation was applied with 35% collision energy, and resulting fragments were detected using the normal scan rate in the ion trap. The raw chromatogram was extracted with m/z of the TMT labeled parent peptide (1259.13-1259.15) and the cyclic asparagine fragment (1022.97-1022.99), both observed in the data of red blood cell lysates.

### Cell Viability Assay

2.0×10⁴ MM.1S cells were seeded in each well of a 96-well plate containing 100 μL RPMI160 media supplemented with 10% FBS and 1× penicillin-streptomycin. The compound of interest was added to each well to a final concentration of 10 nM-100 μM from 100× stock solutions in 4% DMSO/PBS (1 μL). Samples were incubated at 37° C., 5% CO₂ for 5 days. Each well was treated with 3-(4,5-Dimethylthiazol-2-yl)-2,5-diphenyltetrazolium (MTT, 4 mg/mL, 10 μL), and the treated plate was incubated at 37° C., 5% CO₂ for 3 h. The formazan crystals were solubilized by the addition of 100 μL of 10% SDS, 0.01 M HCl per well, and the plate was allowed to incubate at 37° C. overnight. The absorbance at 570 nm was measured to quantify the formazan generated in each well. The blank was defined by wells containing media and MTT reagent without any cells. For each treatment well, the cell viability was calculated by subtracting the blank value and normalizing to the average absorbance of the vehicle control wells (cells treated with 4% DMSO/PBS).

### RT-qPCR

Cells were treated with the indicated drugs in biological triplicate and collected as described in the general procedures. Total RNA was extracted using the Monarch total RNA miniprep kit with on-column DNase digestion. Reverse transcription and real-time PCR were performed using the Luna universal one-step RT-qPCR kit using the supplied protocol. The relative mRNA level was calculated using the 2^{(-ΔΔCt)} method with B-actin as the reference gene. Primers 7-14 were used.

### Time-Course Study of Formation of Cyclic Imide and its Hydrolysis Products

The synthetic peptides (20 μL, 5 mM stock in ddH2O) were incubated in ammonium acetate buffer (380 μL, 20 mM) at pH 7.4, 8.0, or 9.0. Samples were incubated in a sand bath pre-heated to 37° C., and 30 μL was taken from each sample at t=0 h (collected immediately after resuspension in pH 7.4 buffer), 24 h, 48 h, 72 h, 96 h, 120 h, 144 h, 168 h, 192 h and 240 h. Immediately after collection, the samples were quenched by the addition of 0.75 μL of 10% formic acid and stored at −80° C. until analysis. The MS samples were prepared by mixing 2 μL of the peptides, 18 μL 0.1% formic acid, and 20 μL acetonitrile. Peptides were injected on a Thermo Orbitrap Fusion Lumos Tribrid or LTQ Orbitrap Velos and eluted using a multi-step gradient at a flow rate of 0.2 L/min over 60 min (0-5 min, 5% acetonitrile in 0.1% formic acid/water; 5-52 min, 5-80%; 52-55 min, 80-98%; 55-60 min, 98%). The electrospray ionization voltage was set to 2 kV and the capillary temperature was set to 275° C. MS1 scans were performed over 400-2000 m/z at resolution 120,000. HCD fragmentation was performed on the top ten most abundant precursors exhibiting a charge state from one to five at a resolving power setting of 50,000 or 60,000 and fragmentation energy of 37 or 38% in the Orbitrap. CID fragmentation was applied with 35% collision energy, and resulting fragments were detected using the normal scan rate in the ion trap. The raw chromatograms were extracted with m/z of label-free parent peptide (1030.47-1030.49), cyclic imide (908.39-908.41), and hydrolysis products (917.39-917.41) for HBB[42-60]; parent peptide (978.03-978.05), cyclic imide (856.46-856.48), and hydrolysis products (865.46-865.48) for ACTB[96-113]; parent peptide (999.83-999.85), cyclic imide (867.90-867.92 or 685.34-685.36), and hydrolysis products (876.90-876.92 or 703.35-703.37) for HBA[63-91]. For each species, only the peak area at the expected retention time was integrated and quantified using Genesis peak detection on Xcalibur Qual Browser version 3.0.63. The relative log 2 ratio of cyclic imide or hydrolysis products to parent peptide were calculated by subtracting the log 2 ratio at t=0 h from those at other time points.

### Proteomics for Cyclic Imide Detection in HEK293T Cells

Samples were prepared in biological triplicate for each condition. 3×10⁶ WT HEK293T cells or CRBN-KO HEK293T cells with the same passage number (p19) were collected in separate tubes. For lenalidomide treatment, 1.5×10⁶ WT HEK293T cells (p19) were seeded in 6-well plates and incubated at 37° C. for 1 h. The cells were treated with 200 μM lenalidomide for 24 h first. Then, the media was aspirated and fresh media containing 200 μM lenalidomide was added into each well and incubated for another 24 h (48 h treatment in total). After protein quantification by BCA assay, the lysates were diluted to 1 mg/mL with the lysis buffer. To the diluted lysate (100 μL), 0.5 μg of GFP-LPETG (SEQ ID NO: 5) was added as an internal standard (0.42 μL of 43 μM TBS stock), and the mixtures were taken for reduction and alkylation. The samples were loaded on S-trap micro columns similarly as “Global Quantitative Proteomics Sample Preparation”. To digest the S-trap-bound proteins, 2.5 μg of trypsin in 40 μL 50 mM TEAB pH 7.4 was added to each column and incubated at 47° C. for 1 h without rotation. TMT labeling was performed as described in “Proteomics for Cyclic Imide Detection in Red Blood Cells and Bovine Lens”. The combined sample after TMT labeling was resuspended in 900 μL 0.1% TFA and 300 μL was taken to fractionation into 18 fractions using the Pierce high pH reversed-phase peptide fractionation kit. The peptides were eluted sequentially by 5%, 6%, 8%, 10%, 11-20% (with 1% increments), 25%, 30%, 35% and 50% acetonitrile/0.1% TEA. Immediately after the elution, each fraction was acidified by the addition of 5 μL of 10% formic acid. The fractions were concentrated to dryness and each sample was resuspended in 20 μL of 0.1% formic acid prior to LC-MS/MS analysis.

Proteomics for Cyclic Imide Detection in MM.1S cells

Samples were prepared in biological quadruplicate for each condition. 2×10⁶ MM.1S cells (p21) were seeded in 6-well plates and incubated at 37° C. for 1 h. The cells were treated with DMSO or 200 μM lenalidomide for 24 h first. Then, the cells were centrifugated at 300×g, 24° C., 4 min and the media aspirated. Cells were reseeded in 6-well plates with fresh media containing DMSO or 200 μM lenalidomide and incubated for another 24 h (48 h treatment in total). After protein quantification by BCA protein assay, the lysates were diluted to 1 mg/mL with the lysis buffer. To the diluted lysate (100 μL) was added 0.5 μg of GFP-LPETG (SEQ ID NO: 5) as the internal standard (0.42 μL of 43 μM TBS stock), and the mixtures were taken to reduction and alkylation. Digestion, TMT labeling and fractionation were performed as described in “Proteomics for Cyclic Imide Detection in HEK293T cells”.

### Proteomics Mass Spectrometry Acquisition Procedures for Cyclic Imide Detection

Desalted and fractionated samples were resuspended in 0.1% formic acid/water (20 μL per sample). The sample (2.0 μL) was loaded onto a C18 trap column (3 cm, 3 μm particle size C10 Dr. Maisch 150 μm I.D) and then separated on an analytical column (Thermo Scientific Acclaim PepMap 100, 2 μm particle size, 250 mm length, 75 μm internal diameter) at 0.2 μL/min with a Thermo Scientific Ultimate 3000 system connected in line to a Thermo Scientific Orbitrap Fusion Lumos Tribrid. The column temperature was maintained at 50° C. Peptides were eluted using a multi-step gradient at a flow rate of 0.2 μL/min over 90 min (0-5 min, 2% acetonitrile in 0.1% formic acid/water; 5-7 min, 2-5%; 7-75 min, 5-40%; 75-85 min, 40-95%; 85-90 min, 95%) for HEK293T and MM.1S and 180 min (0-5 min, 2% acetonitrile in 0.1% formic acid/water; 5-15 min, 2-10%; 15-160 min, 10-40%; 160-170 min, 40-95%; 170-180 min, 95%) for RBC and bovine lens. The electrospray ionization voltage was set to 2 kV and the capillary temperature was set to 275° C. Dynamic exclusion was enabled with a mass tolerance of 10 ppm and exclusion duration of 90 sec. MS1 scans were performed over 410-2000 m/z at resolution 120,000. HCD fragmentation was performed on the top ten most abundant precursors exhibiting a charge state from two to five at a resolving power setting of 60,000 and fragmentation energy of 37% in the Orbitrap. CID fragmentation was applied with 35% collision energy, and resulting fragments were detected using the normal scan rate in the ion trap.

### Mass Spectrometry Data Analysis for Cyclic Imide Detection

Analysis was performed in Thermo Scientific Proteome Discoverer version 2.4.1.15. The raw data were searched against SwissProt human (Homo sapiens) protein database (21 Feb. 2019; 20,355 total entries) or SwissProt bovine (Bos taurus) protein database (19 Aug. 2016; 5,997 entries) and contaminant proteins using the Sequest HT algorithm. Searches were performed with the following guidelines: spectra with a signal-to-noise ratio greater than 1.5; mass tolerance of 10 ppm for the precursor ions and 0.02 Da (HCD) and 0.6 Da (CID) for fragment ions; semi-tryptic digestion with specificity at the peptide N-terminus only; 2 missed cleavages; variable oxidation on methionine residues (+15.995 Da); static carboxyamidomethylation of cysteine residues (+57.021 Da); static TMT labeling (+226.163 Da) at lysine residues and N-termini; variable dehydration on asparagine and glutamine residues at C-terminus only (−18.015 Da). The TMTreporter ions were quantified using the Reporter Ions Quantifier node and normalized to the intensity of GFP-LPETG (SEQ ID NO: 5) peptides for HEK293T and MM.1S and the summed peptide intensity for RBC and bovine lens. Peptide spectral matches (PSMs) were filtered using a 1% or 5% FDR using Target Decoy PSM validator. For the obtained peptide groups, the data were further filtered to include only peptides with 1% or 5% FDR, bearing the modification of dehydration on asparagine or glutamine residues, and derived from non-contaminant proteins to generate the list of peptides bearing C-terminal cyclic imide modifications. To generate the list of peptides bearing C-terminal glutamine or asparagine, the data were filtered to include only peptides with 1% or 5% FDR, bearing asparagine or glutamine residues at the peptide C-terminus, and derived from noncontaminant proteins. The peptides that were mapped back to the protein C-terminus were excluded. The p-value and fold change calculations were performed using the algorithm from Grouping and Quantification on Proteome Discoverer. Standard deviations of the grouped abundance were calculated by the multiplication of grouped abundance and CV % which were both obtained from Proteome Discoverer.

Identification of C-Terminal Cyclic Imide Modification Sites from Public Datasets

Data were analyzed as described under “Mass Spectrometry Data Analysis for Cyclic Imide Detection” with the following modifications. For the analysis of global proteomics datasets obtained from CPTAC, the RAW datasets were processed according to the methods in the respective publication for fully tryptic peptides and spectra that did not receive a confident peptide spectral match were searched for N-terminal semi-tryptic sequences and an additional dynamic modification of dehydration on asparagine or glutamine residues (−18.015 Da) at the C-terminus of the peptide. The data were filtered with a 1% FDR using Percolator.

### Synthetic Procedures

### General Reagent Information

All reactions were performed under air using the indicated method in general procedures unless otherwise noted. N,N-Dimethylformamide (DMF) (Beantown Chemical, catalog no. BT138690) and tetrahydrofuran (THF) (Fisher Chemical, catalog no. T427-4) were vigorously purged with argon for 1 h, followed by passage under argon pressure through two packed columns of neutral alumina (Pure Process Technology). Milli-Q (MQ) water was prepared using a Barnstead™ GenPure™ xCAD Plus Ultrapure Water Purification System (Thermo Fisher Scientific). CD₃OD, DMSO-d₆, and acetone-do were purchased from Cambridge Isotope Laboratories. dFKBP-1 was synthesized as previously described and NMR spectra were matched with reported data.¹¹³ dCDK6-Pom was synthesized as previously described and NMR spectra were matched with reported data (N. A. Anderson, et al. Bioorg. Med. Chem. Lett. 30, 127106 (2020)). Other solvents and reagents were either prepared according to referenced literature procedures or were purchased from chemical suppliers (Sigma Aldrich, Alfa Aesar, AstaTech, Chem-Impex International, Inc., Strem Chemicals Inc., Acros Organics, TCI, Combi-Blocks, Matrix Scientific, Oakwood Chemical, Thermo Fisher Scientific, A1 BioChem Labs, Cayman Chemical Company, VWR Chemicals BDH®, or J. T. Baker) and were used as received unless otherwise noted. Flash Column Chromatography was performed using silica gel purchased from Silicycle (SilicaFlash® F60, 40-63 μm) with the aid of a CombiFlash® NextGen 300+ Automated Flash Chromatography System (Teledyne ISCO). Organic solutions were concentrated in vacuo using a Buchi rotary evaporator. Analytical thin-layer chromatography (TLC) was performed using glass plates pre-coated with silica gel (0.25 mm, 60 Å pore size) impregnated with a fluorescent indicator (254 nm). TLC plates were visualized by exposure to ultraviolet light (UV), iodine (I₂), and/or submersion in p-anisaldehyde followed by brief heating with a heat gun (10-15 sec).

### General Analytical Information

All compounds were characterized by ¹H NMR and ¹³C NMR. New compounds were also characterized by IR spectroscopy and high-resolution mass spectroscopy. NMR experiments were performed on a Bruker 400 MHZ, Varian 500 MHZ, or Agilent 600 MHz instrument at 24° C. Chemical shifts are reported in parts per million (ppm) relative to residual solvent as an internal reference (CD₂HOD: δ 3.31 ppm for ¹H and δ 49.00 ppm for ¹³C; DMSO: 2.50 ppm for ¹H and 39.52 ppm for ¹³C; acetone: δ 2.05 ppm for ¹H and 29.84 ppm for ¹³C). The following abbreviations were used to explain multiplicities: s=singlet, bs=broad singlet, d=doublet, t=triplet, q=quartet, dd=doublet of doublets, m=multiplet. ¹³C NMR spectra were obtained with ¹H decoupling. IR spectra were recorded on a Bruker ALPHA FT-IR and are reported in terms of frequency of absorption (cm⁻¹) and intensity of absorption (s=strong, m=medium, w=weak, br=broad). High-resolution mass spectra were recorded on a Bruker micro TOF-Q II hybrid quadrupole-time of flight system and on an Agilent 1260 UPLC-MS system. Low-resolution mass spectra were obtained on a Waters ACQUITY UPLC system equipped with SQ Detector 2 mass spectrometer.

### General Procedure A: Synthesis of JQ1-Linker

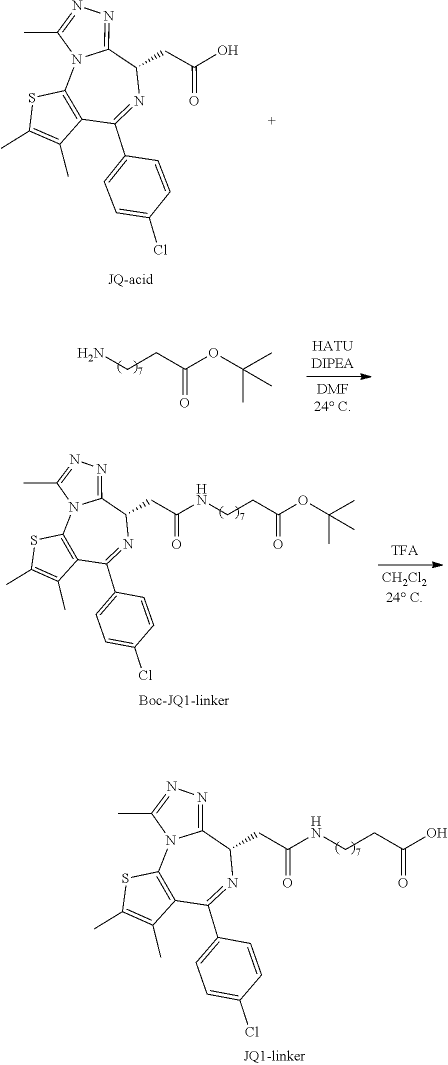

JQ-acid¹¹³ (677 mg, 1.69 mmol, 1.00 equiv) and tert-butyl 9-aminononanoate (229 mg, 1.86 mmol, 1.10 equiv) were dissolved in dry DMF (16.9 mL, 0.1 M). N,N-Diisopropylethyl amine (1.47 mL, 8.44 mmol, 5.00 equiv) and HATU (642 mg, 1.69 mmol, 1.00 equiv) were added in sequence to the stirred reaction mixture. After stirring at 24° C. for 18 h, the reaction mixture was diluted with ethyl acetate (20 mL) and transferred to a separatory funnel. The organic layer was washed sequentially with brine (100 mL), 10% aqueous citric acid (50 mL), and saturated aqueous NaHCO₃ (50 mL). The organic layer was dried over Na₂SO₄, filtered, and concentrated with the aid of a rotary evaporator. The residue obtained was purified by column chromatography (ISCO, 80 g column, 0-5% MeOH/CH₂Cl₂, 30 min gradient) to afford Boc-JQ1-linker as a cream colored solid (374 mg, 0.611 mmol, 36% yield).

¹H NMR (500 MHz, CD₃OD): δ 7.45 (d, J=8.5 Hz, 2H), 7.39 (d, J=8.7 Hz, 2H), 4.63 (dd, J=9.1, 5.1 Hz, 1H), 3.41 (dd, J=14.9, 9.1 Hz, 1H), 3.29-3.17 (m, 3H), 2.69 (s, 3H), 2.43 (s, 3H), 2.19 (t, J=7.4 Hz, 2H), 1.69 (s, 3H), 1.60-1.51 (m, 4H), 1.43 (s, 9H), 1.38-1.29 (m, 8H). ¹³C NMR (126 MHz, CD₃OD): δ 175.0, 172.6, 166.1, 157.0, 152.1, 138.1, 137.9, 133.5, 133.2, 132.0, 132.0, 131.3, 129.7, 81.2, 55.2, 40.5, 38.8, 36.4, 30.5, 30.4, 30.3, 30.1, 28.4, 28.0, 26.2, 14.5, 13.0, 11.6. IR (ATR-FTIR) 3297 (br), 2927 (m), 1725 (m), 1654 (m), 1530 (m), 1418 (m), 1365 (m), 1149 (s), 1089 (m), 1014 (w), 840 (m), 732 (s). HRMS (ESI) (m/z): [M+H]⁺ calculated for C₃₂H₄₃ClN₅O₃S, 612.2770. found, 612.2761.

Boc-JQ1-linker (30.6 mg, 50.0 μmol, 1.00 equiv) was dissolved in a solution of TFA (0.50 mL) and CH₂Cl₂ (0.25 mL). After 1 h, the reaction mixture was concentrated with the aid of a rotary evaporator and dried under high vacuum to yield JQ1-linker as a yellow oil (26.8 mg, 48.2 μmol, 96% yield), which was used directly in the following step without further purification.

### General Procedure B: Synthesis of FcQ

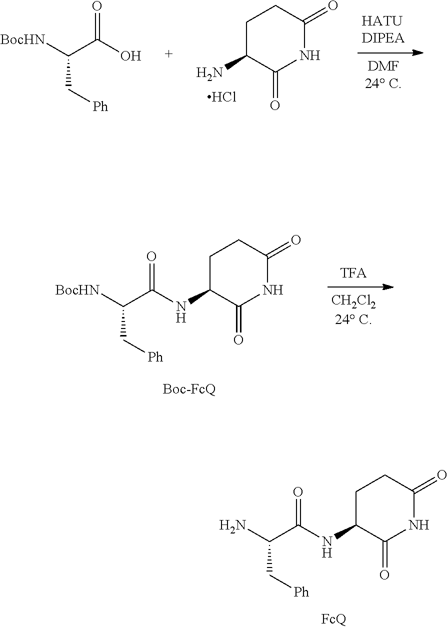 [SMILES: CC(C)(C)OC(=O)N[C@@H](Cc1ccccc1)C(=O)O ; Cl ; N[C@H]1CCC(=O)NC1=O ; CC(C)(C)OC(=O)N[C@@H](Cc1ccccc1)C(=O)N[C@H]1CCC(=O)NC1=O ; N[C@@H](Cc1ccccc1)C(=O)N[C@H]1CCC(=O)NC1=O]

Boc-L-phenylalanine (531 mg, 2.00 mmol, 1.00 equiv) and (S)-3-aminopiperidine-2,6-dione hydrochloride/14 (346 mg, 2.10 mmol, 1.05 equiv) were dissolved in dry DMF (10 mL, 0.2 M). N,N-Diisopropylethyl amine (1.74 mL, 10.0 mmol, 5.00 equiv) and HATU (761 mg, 2.00 mmol, 1.00 equiv) were added in sequence to the stirred reaction mixture. After stirring at 24° C. for 18 h, the reaction mixture was diluted with ethyl acetate (30 mL) and washed sequentially with brine (100 mL), 10% aqueous citric acid (50 mL), and saturated aqueous NaHCO₃ (50 mL). The organic layer was dried over Na₂SO₄, filtered, and concentrated with the aid of a rotary evaporator. The residue obtained was purified by column chromatography (ISCO, 12 g column, 0-10% MeOH/CH₂Cl₂, 30 min gradient) to give Boc-FcQ as a white solid (468 mg, 1.25 mmol, 62% yield). NMR spectra were matched with reported data.¹¹⁵ Boc-FcQ (18.8 mg, 50.0 μmol, 1.00 equiv) was dissolved in TFA (0.50 mL) and CH₂Cl₂ (0.50 mL). After 1 h, the reaction mixture was concentrated with the aid of a rotary evaporator and dried under high vacuum to yield FcQ as a yellow oil, which was used directly in the following step without further purification.

### General Procedure C: Synthesis of JQ1-XcQ

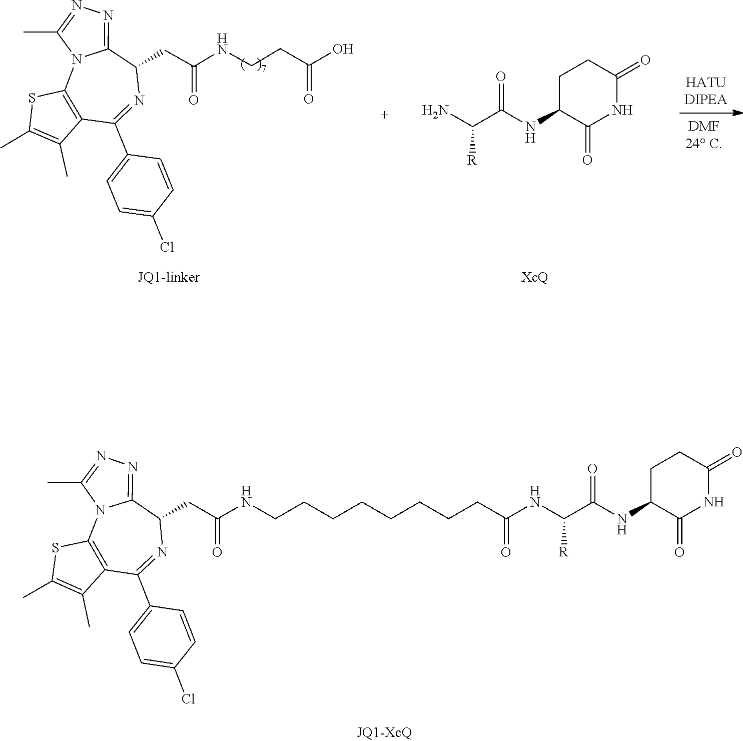

XcQ was prepared according to General Procedure B from the respective Boc-protected amino acid (1.00 equiv) and (S)-3-aminopiperidine-2,6-dione hydrochloride (1.05 equiv). JQ1-linker (1.00 equiv) and XcQ (1.00-2.10 equiv) were dissolved in dry DMF (0.048 M). N,N-Diisopropylethyl amine (5.00 equiv) and HATU (1.00 equiv) were added in sequence to the stirred reaction mixture. After stirring at 24° C. for 18-24 h, the reaction mixture was diluted with ethyl acetate and washed sequentially with brine, 10% aqueous citric acid, and saturated aqueous NaHCO₃. The organic layer was dried over Na₂SO₄, filtered, and concentrated with the aid of a rotary evaporator. The residue obtained was purified by column chromatography to afford JQ1-XcQ.

### JQ1-GcQ

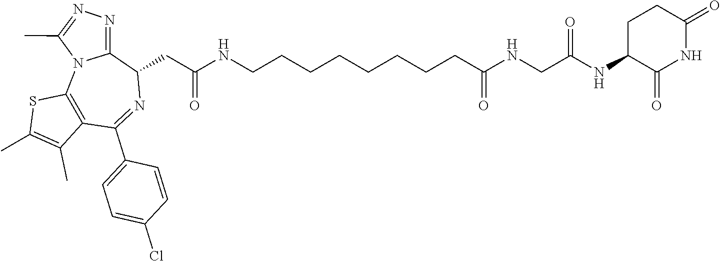 [SMILES: Cc1sc2c(c1C)C(c1ccc(Cl)cc1)=N[C@@H](CC(=O)NCCCCCCCCC(=O)NCC(=O)N[C@H]1CCC(=O)NC1=O)c1nnc(C)n1-2]

The title compound was prepared according to General Procedure C from JQ1-linker (12.4 mg, 22.3 μmol, 1.00 equiv), GcQ (4.1 mg, 22.3 μmol, 1.00 equiv), N,N-diisopropylethyl amine (20 μL, 111 μmol, 5.00 equiv), and HATU (9.2 mg, 24.5 μmol, 1.10 equiv) in dry DMF (0.46 mL, 0.048 M). After purification by column chromatography (ISCO, 4 g column, 0-10% MeOH/CH₂Cl₂, 45 min gradient), the title compound, JQ1-GcQ, was obtained as a white solid (3.5 mg, 4.83 μmol, 22% yield).

¹H NMR (500 MHZ, CD₃OD) δ 7.46 (d, J=8.6 Hz, 2H), 7.41 (d, J=8.7 Hz, 2H), 4.68-4.58 (m, 2H), 3.91 (d, J=2.1 Hz, 2H), 3.41 (dd, J=14.9, 9.0 Hz, 1H), 3.30-3.21 (m, 3H), 2.80-2.72 (m, 1H), 2.70 (s, 3H), 2.69-2.62 (m, 1H), 2.45 (s, 3H), 2.27 (t, J=7.5 Hz, 2H), 2.20-2.14 (m, 1H), 2.06-1.96 (m, 1H), 1.70 (s, 3H), 1.66-1.54 (m, 4H), 1.38-1.30 (m, 8H). ¹³C NMR (101 MHz, DMSO-d₆) δ 172.9, 172.5, 172.4, 172.1, 169.3, 163.0, 155.1, 149.8, 136.7, 135.2, 132.3, 130.7, 130.1, 129.8, 129.6, 128.5, 53.9, 51.8, 49.1, 41.8, 38.5, 37.7, 35.2, 30.9, 29.3, 28.8, 28.7, 26.4, 25.1, 24.3, 14.1, 12.7, 11.3. IR (ATR-FTIR) 3296 (br), 2922 (s), 2852 (m), 1650 (s), 1549 (s), 1418 (m), 1378 (m), 1262 (m), 1089 (m), 1014 (m), 804 (w), 732 (m). HRMS (ESI) (m/z) [M+H]⁺ calculated for C₃₅H₄₄ClN₈O₅S, 723.2838. found, 723.2827.

### JQ1-ACQ

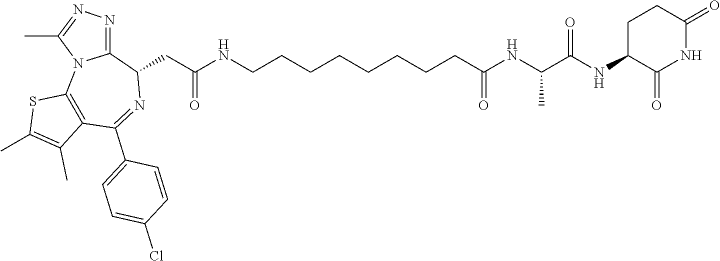 [SMILES: Cc1sc2c(c1C)C(c1ccc(Cl)cc1)=N[C@@H](CC(=O)NCCCCCCCCC(=O)N[C@@H](C)C(=O)N[C@H]1CCC(=O)NC1=O)c1nnc(C)n1-2]

The title compound was prepared according to General Procedure C from JQ1-linker (18.2 mg, 32.7 μmol, 1.00 equiv), AcQ (9.3 mg, 39.3 μmol, 1.20 equiv), N,N-diisopropylethyl amine (28.5 μL, 164 μmol, 5.00 equiv), and HATU (13.7 mg, 35.9 μmol, 1.10 equiv) in dry DMF (0.70 mL, 0.047 M). After purification by column chromatography (ISCO, 4 g column, 0-10% MeOH/CH₂Cl₂, 45 min gradient), the title compound, JQ1-AcQ, was obtained as an off-white white solid (15.4 mg, 20.9 μmol, 64% yield).

¹H NMR (500 MHZ, CD₃OD) δ 7.45 (d, J=8.4 Hz, 2H), 7.40 (d, J=8.4 Hz, 2H), 4.66-4.58 (m, 2H), 4.38 (q, J=7.1 Hz, 1H), 3.41 (dd, J=14.9, 9.0 Hz, 1H), 3.30-3.19 (m, 3H), 2.79-2.71 (m, 1H), 2.70 (s, 3H), 2.68-2.62 (m, 1H), 2.45 (s, 3H), 2.23 (t, J=7.5 Hz, 2H), 2.19-2.14 (m, 1H), 2.04-1.94 (m, 1H), 1.70 (s, 3H), 1.65-1.53 (m, 4H), 1.42-1.33 (m, 11H). ¹³C NMR (101 MHZ, Acetone-d₆) δ 173.5, 173.2, 172.8, 172.4, 170.6, 164.1, 156.5, 150.6, 138.3, 136.7, 133.6, 131.7, 131.2, 131.1, 131.1, 129.3, 55.3, 50.8, 49.4, 39.6, 39.2, 36.4, 31.8, 30.4, 29.8, 29.5, 29.5, 27.2, 26.1, 25.5, 18.6, 14.6, 13.0, 11.8. IR (ATR-FTIR) 3272 (br s), 2923 (m), 2852 (m), 1643 (s), 1528 (s), 1418 (m), 1358 (m), 1245 (m), 1192 (s), 1089 (m), 1013 (w), 840 (w). HRMS (ESI) (m/z) [M+H]⁺ calculated for C₃₆H₄₅ClN₈O₅S, 737.2995. found, 737.2987.

### JQ1-LcQ

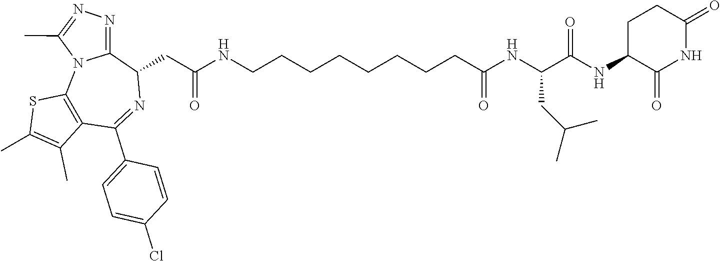 [SMILES: Cc1sc2c(c1C)C(c1ccc(Cl)cc1)=N[C@@H](CC(=O)NCCCCCCCCC(=O)N[C@@H](CC(C)C)C(=O)N[C@H]1CCC(=O)NC1=O)c1nnc(C)n1-2]

The title compound was prepared according to General Procedure C from JQ1-linker (10.0 mg, 18.0 μmol, 1.00 equiv), LcQ (8.7 mg, 36.0 μmol, 2.00 equiv), N,N-diisopropylethyl amine (16 μL, 90.0 μmol, 5.00 equiv), and HATU (13.7 mg, 36.0 μmol, 2.00 equiv) in dry DMF (0.36 mL, 0.050 M). After purification by column chromatography (ISCO, 4 g column, 0-5% MeOH/CH₂Cl₂, 45 min gradient), the title compound, JQ1-LcQ, was obtained as a white solid (6.1 mg, 7.83 μmol, 43% yield).

¹H NMR (400 MHZ, CD₃OD) δ 7.46 (d, J=8.7 Hz, 2H), 7.40 (d, J=8.7 Hz, 2H), 4.67-4.58 (m, 2H), 4.43 (dd, J=9.1, 6.0 Hz, 1H), 3.41 (dd, J=14.9, 9.0 Hz, 1H), 3.29-3.22 (m, 2H), 2.80-2.71 (m, 1H), 2.70 (s, 3H), 2.69-2.61 (m, 1H), 2.45 (s, 3H), 2.27-2.21 (m, 2H), 2.20-2.11 (m, 1H), 2.08-1.94 (m, 1H), 1.80-1.71 (m, 1H), 1.71 (s, 3H), 1.66-1.52 (m, 6H), 1.40-1.35 (m, 9H), 0.96 (dd, J=15.1, 6.4 Hz, 6H). ¹³C NMR (101 MHz, CD₃OD) δ 176.3, 175.2, 174.8, 173.2, 172.7, 166.2, 157.1, 152.2, 138.1, 138.0, 133.5, 133.3, 132.0, 132.0, 131.3, 129.8, 55.3, 53.1, 51.1, 42.0, 40.5, 38.8, 36.8, 32.0, 30.5, 30.4, 30.3, 30.2, 28.0, 26.9, 25.9, 25.6, 23.5, 22.0, 14.4, 12.9, 11.6. IR (ATR-FTIR) 3320 (br), 2918 (s), 2850 (m), 1735 (m), 1654 (m), 1541 (m), 1457 (m), 1377 (m), 1197 (m), 1091 (m), 844 (w), 803 (w). HRMS (ESI) (m/z) [M+H]⁺ calculated for C₃₉H₅₂ClN₈O₅S, 779.3464. found, 779.3456.

### JQ1-IcQ

The title compound was prepared according to General Procedure C from JQ1-linker (10.0 mg, 18.0 μmol, 1.00 equiv), IcQ (8.7 mg, 36.0 μmol, 2.00 equiv), N,N-diisopropylethyl amine (16 μL, 90.0 μmol, 5.00 equiv), and HATU (7.5 mg, 20.0 μmol, 1.10

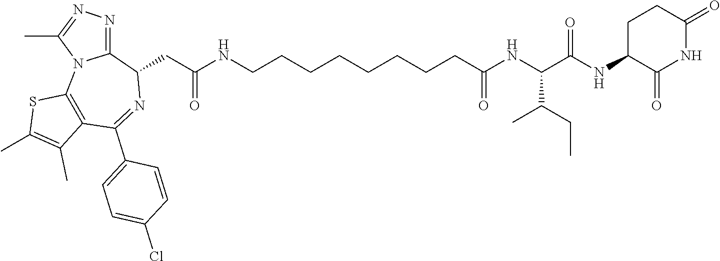 [SMILES: CCC(C)[C@H](NC(=O)CCCCCCCCNC(=O)C[C@@H]1N=C(c2ccc(Cl)cc2)c2c(sc(C)c2C)-n2c(C)nnc21)C(=O)N[C@H]1CCC(=O)NC1=O]

equiv) in dry DMF (0.36 mL, 0.050 M). After purification by column chromatography (ISCO, 4 g column, 0-5% MeOH/CH₂Cl₂, 45 min gradient), the title compound, JQ1-IcQ, was obtained as a white solid (5.5 mg, 7.06 μmol, 39% yield).

¹H NMR (400 MHZ, CD₃OD) δ 7.46 (d, J=8.7 Hz, 2H), 7.40 (d, J=8.7 Hz, 2H), 4.67-4.60 (m, 2H), 4.25 (d, J=7.8 Hz, 1H), 3.41 (dd, J=14.9, 9.0 Hz, 1H), 3.29-3.20 (m, 2H), 2.81-2.71 (m, 1H), 2.70 (s, 3H), 2.69-2.61 (m, 1H), 2.45 (s, 3H), 2.30-2.21 (m, 2H), 2.18-2.11 (m, 1H), 2.07-1.95 (m, 1H), 1.92-1.80 (m, 1H), 1.70 (s, 3H), 1.64-1.54 (m, 5H), 1.40-1.31 (m, 9H), 1.00 (d, J=6.8 Hz, 3H), 0.94-0.89 (m, 4H). ¹³C NMR (101 MHZ, CD₃OD) δ 176.2, 174.8, 174.0, 172.9, 172.7, 166.2, 157.1, 152.2, 138.1, 138.0, 133.5, 133.3, 132.0, 132.0, 131.3, 129.8, 59.2, 55.3, 51.0, 40.5, 38.8, 38.0, 36.8, 32.0, 30.5, 30.4, 30.3, 30.2, 27.9, 27.0, 26.0, 25.6, 15.9, 14.4, 12.9, 11.6, 11.3. IR (ATR-FTIR) 3306 (br), 2918 (s), 2850 (m), 1717 (m), 1654 (m), 1541 (m), 1377 (m), 1195 (m), 1091 (m), 952 (w), 844 (m), 803 (w). HRMS (ESI) (m/z) [M+H]⁺ calculated for C₃₉H₅₂ClN₈O₅S, 779.3464. found, 779.3458.

### JQ1-VcQ

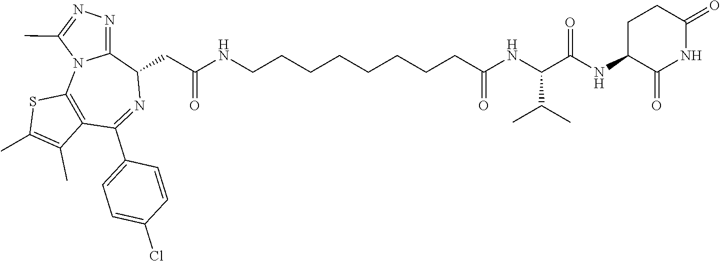 [SMILES: Cc1sc2c(c1C)C(c1ccc(Cl)cc1)=N[C@@H](CC(=O)NCCCCCCCCC(=O)N[C@H](C(=O)N[C@H]1CCC(=O)NC1=O)C(C)C)c1nnc(C)n1-2]

The title compound was prepared according to General Procedure C from JQ1-linker (23.0 mg, 41.4 μmol, 1.00 equiv), VcQ (11.3 mg, 49.6 μmol, 1.20 equiv), N,N-diisopropylethyl amine (36 μL, 207 μmol, 5.00 equiv), and HATU (17.3 mg, 45.5 μmol, 1.10 equiv) in dry DMF (0.90 mL, 0.046 M). After purification by column chromatography (ISCO, 4 g column, 0-10% MeOH/CH₂Cl₂, 45 min gradient), the title compound, JQ1-VcQ, was obtained as a white solid (21.2 mg, 27.7 μmol, 69% yield).

¹H NMR (500 MHZ, CD₃OD) δ 7.45 (d, J=8.4 Hz, 2H), 7.40 (d, J=8.3 Hz, 2H), 4.67-4.60 (m, 2H), 4.21 (d, J=7.2 Hz, 1H), 3.41 (dd, J=14.9, 9.0 Hz, 1H), 3.28-3.20 (m, 2H), 2.79-2.71 (m, 1H), 2.70 (s, 3H), 2.68-2.62 (m, 1H), 2.45 (s, 3H), 2.30-2.21 (m, 2H), 2.18-2.06 (m, 2H), 2.06-1.96 (m, 1H), 1.70 (s, 3H), 1.64-1.53 (m, 4H), 1.39-1.32 (m, 9H), 1.02 (d, J=6.8 Hz, 3H), 0.98 (d, J=6.7 Hz, 3H). ¹³C NMR (126 MHz, CD₃OD) δ 176.3, 174.8, 173.9, 173.0, 172.7, 166.2, 157.0, 152.2, 138.1, 138.0, 133.5, 133.3, 132.0, 132.0, 131.3, 129.8, 60.2, 55.3, 51.0, 40.5, 38.8, 36.8, 32.0, 31.9, 30.5, 30.4, 30.3, 30.2, 27.9, 27.0, 25.6, 19.8, 18.8, 14.4, 12.9, 11.6. IR (ATR-FTIR) 3293 (br), 2925 (s), 2854 (m), 1708 (m), 1647 (s), 1541 (s), 1419 (m), 1252 (w), 1195 (m), 1089 (m), 1014 (w), 732 (w). HRMS (ESI) (m/z) [M+H]⁺ calculated for C₃₈H₅₀ClN₈O₅S, 765.3308. found, 765.3300.

### JQ1-McQ

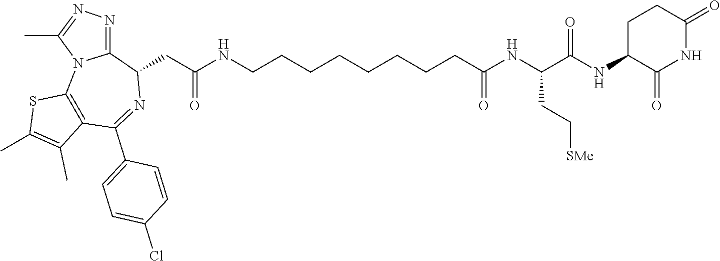 [SMILES: CSCC[C@H](NC(=O)CCCCCCCCNC(=O)C[C@@H]1N=C(c2ccc(Cl)cc2)c2c(sc(C)c2C)-n2c(C)nnc21)C(=O)N[C@H]1CCC(=O)NC1=O]

The title compound was prepared according to General Procedure C from JQ1-linker (10.0 mg, 17.9 μmol, 1.00 equiv), McQ (6.4 mg, 21.6 μmol, 1.20 equiv), N,N-diisopropylethyl amine (15.7 μL, 89.9 μmol, 5.00 equiv), and HATU (7.5 mg, 19.8 μmol, 1.10 equiv) in dry DMF (0.38 mL, 0.047 M). After purification by column chromatography (ISCO, 4 g column, 0-10% MeOH/CH₂Cl₂, 45 min gradient), the title compound, JQ1-McQ, was obtained as a white solid (7.2 mg, 9.03 μmol, 50% yield).

¹H NMR (400 MHZ, CD₃OD) δ 7.46 (d, J=8.6 Hz, 2H), 7.40 (d, J=8.6 Hz, 2H), 4.67-4.60 (m, 2H), 4.50 (dd, J=8.4, 5.5 Hz, 1H), 3.41 (dd, J=14.9, 9.0 Hz, 1H), 3.29-3.19 (m, 2H), 2.81-2.71 (m, 1H), 2.70 (s, 3H), 2.69-2.66 (m, 1H), 2.65-2.52 (m, 2H), 2.45 (s, 3H), 2.25 (t, J=7.4 Hz, 2H), 2.19-2.10 (m, 2H), 2.09 (s, 3H), 2.07-1.91 (m, 2H), 1.70 (s, 3H), 1.66-1.52 (m, 4H), 1.43-1.30 (m, 9H). ¹³C NMR (101 MHZ, CD₃OD) δ 176.3, 174.8, 174.2, 173.2, 172.7, 166.2, 157.1, 152.2, 138.1, 138.0, 133.5, 133.3, 132.0, 132.0, 131.3, 129.8, 55.3, 53.9, 51.1, 40.5, 38.8, 36.8, 32.9, 32.1, 31.0, 30.5, 30.3, 30.3, 30.2, 27.9, 26.8, 25.5, 15.3, 14.4, 12.9, 11.6. IR (ATR-FTIR) 3291 (br), 2925 (m), 1708 (m), 1644 (s), 1532 (s), 1419 (m), 1265 (m), 1194 (m), 1089 (m), 1014 (w), 731 (s), 701 (m). HRMS (ESI) (m/z) [M+H]⁺ calculated for C₃₈H₅₀ClN₈O₅S₂, 797.3029. found, 797.3018.

### JQ1-PcQ

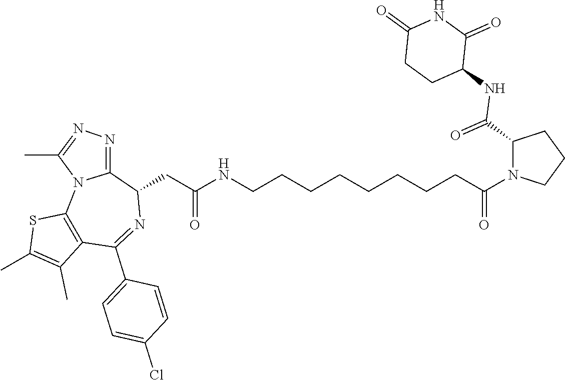 [SMILES: Cc1sc2c(c1C)C(c1ccc(Cl)cc1)=N[C@@H](CC(=O)NCCCCCCCCC(=O)N1CCC[C@H]1C(=O)N[C@H]1CCC(=O)NC1=O)c1nnc(C)n1-2]

The title compound was prepared according to General Procedure C from JQ1-linker (21.1 mg, 37.9 μmol, 1.00 equiv), PcQ (14.9 mg, 45.5 μmol, 1.10 equiv), N,N-diisopropylethyl amine (33 μL, 189 μmol, 5.00 equiv), and HATU (15.9 mg, 41.7 μmol, 1.10 equiv) in dry DMF (0.81 mL, 0.047 M). After purification by column chromatography (ISCO, 4 g column, 0-10% MeOH/CH₂Cl₂, 45 min gradient), the title compound, JQ1-PcQ, was obtained as a white solid (10.7 mg, 14.0 μmol, 38% yield).

¹H NMR (mixture of rotamers, 600 MHz, CD₃OD) δ 7.46 (d, J=8.6 Hz, 2H), 7.41 (d, J=8.0 Hz, 2H), 4.66-4.61 (m, 2H), 4.47 (dd, J=8.7, 2.7 Hz, 0.3H), 4.42 (dd, J=8.5, 3.7 Hz, 0.7H), 3.69-3.62 (m, 1H), 3.60-3.53 (m, 0.9H), 3.53-3.47 (m, 0.3H), 3.44-3.38 (m, 1H), 3.29-3.21 (m, 2H), 2.79-2.72 (m, 1H), 2.70 (s, 3H), 2.69-2.62 (m, 1H), 2.45 (s, 3H), 2.40-2.33 (m, 1.6H), 2.31-2.26 (m, 0.6H), 2.24-2.15 (m, 2H), 2.13-2.05 (m, 2H), 2.04-1.95 (m, 1.6H), 1.95-1.89 (m, 0.6H), 1.70 (s, 3H), 1.64-1.54 (m, 4H), 1.41-1.32 (m, 9H). ¹³C NMR (mixture of rotamers, 101 MHz, DMSO-d₆) δ 173.1, 173.0, 172.2, 172.2, 172.2, 172.1, 171.3, 170.8, 169.4, 163.0, 155.2, 149.9, 136.8, 135.3, 132.3, 130.8, 130.2, 129.8, 129.6, 128.5, 60.0, 59.2, 54.0, 49.0, 46.8, 46.4, 38.45, 37.7, 33.6, 33.3, 31.8, 30.9, 30.9, 30.8, 29.5, 29.3, 29.0, 29.0, 28.8, 26.4, 24.3, 24.3, 24.2, 24.1, 14.1, 12.7, 11.3. IR (ATR-FTIR) 3306 (br), 2926 (m), 2854 (m), 1735 (m), 1654 (s), 1541 (s), 1488 (m), 1419 (m), 1088 (m), 1014 (w), 841 (w), 732 (w). HRMS (ESI) (m/z) [M+H]⁺ calculated for C₃₈H₄₈ClN₈O₅S, 763.3151; found, 763.3144.

### JO1-FcQ

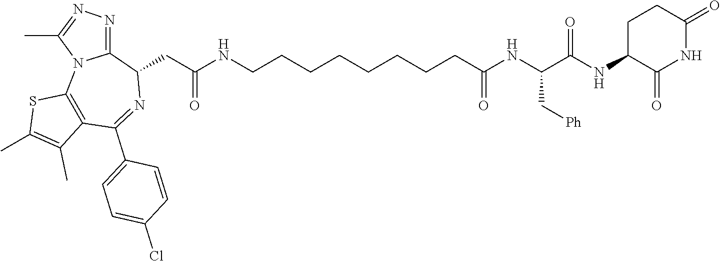 [SMILES: Cc1sc2c(c1C)C(c1ccc(Cl)cc1)=N[C@@H](CC(=O)NCCCCCCCCC(=O)N[C@@H](Cc1ccccc1)C(=O)N[C@H]1CCC(=O)NC1=O)c1nnc(C)n1-2]

The title compound was prepared according to General Procedure C from JQ1-linker (14.2 mg, 25.5 μmol, 1.00 equiv), FcQ (7.7 mg, 28.0 μmol, 1.10 equiv), N,N-diisopropylethyl amine (22 μL, 127 μmol, 5.00 equiv), and HATU (10.6 mg, 28.0 μmol, 1.10 equiv) in dry DMF (0.53 mL, 0.048 M). After purification by column chromatography (ISCO, 4 g column, 0-10% MeOH/CH₂Cl₂, 45 min gradient), the title compound, JQ1-FcQ, was obtained as a white solid (9.0 mg, 11.1 μmol, 44% yield).

¹H NMR (500 MHZ, CD₃OD) δ 7.45 (d, J=8.6 Hz, 2H), 7.40 (d, J=8.8 Hz, 2H), 7.30-7.23 (m, 4H), 7.22-7.14 (m, 1H), 4.71 (dd, J=9.9, 4.8 Hz, 1H), 4.65-4.58 (m, 2H), 3.41 (dd, J=14.9, 9.0 Hz, 1H), 3.28-3.19 (m, 3H), 2.89 (dd, J=14.1, 10.0 Hz, 1H), 2.79-2.71 (m, 1H), 2.69 (s, 3H), 2.68-2.61 (m, 1H), 2.45 (s, 3H), 2.19-2.09 (m, 3H), 2.07-1.96 (m, 1H), 1.70 (s, 3H), 1.61-1.53 (m, 2H), 1.50-1.43 (m, 2H), 1.37-01.14 (m, 9H). ¹³C NMR (101 MHZ, CD₃OD) δ 176.1, 174.8, 174.0, 173.1, 172.7, 166.2, 157.1, 152.2, 138.6, 138.1, 138.0, 133.5, 133.3, 132.0, 132.0, 131.3, 130.4, 129.8, 129.4, 127.7, 55.7, 55.3, 51.2, 40.5, 39.0, 38.8, 36.8, 32.0, 30.5, 30.3, 30.2, 30.0, 27.9, 26.8, 25.6, 14.4, 12.9, 11.6. IR (ATR-FTIR) 3296 (br), 2926 (m), 1646 (s), 1592 (w), 1532 (s), 1419 (m), 1362 (w), 1195 (m), 1089 (m), 1014 (w), 732 (m), 700 (m). HRMS (ESI) (m/z) [M+H]⁺ calculated for C₄₂H₅₀ClN₈O₅S, 813.3308. found, 813.3291.

### JQ1-WcQ

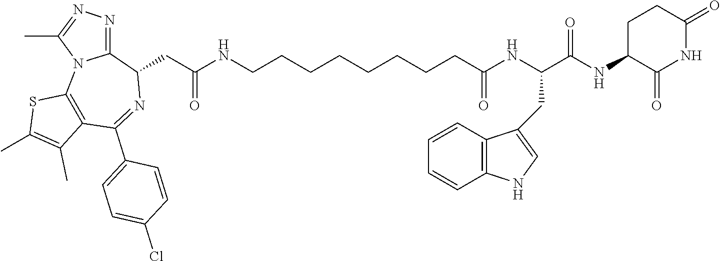 [SMILES: Cc1sc2c(c1C)C(c1ccc(Cl)cc1)=N[C@@H](CC(=O)NCCCCCCCCC(=O)N[C@@H](Cc1c[nH]c3ccccc13)C(=O)N[C@H]1CCC(=O)NC1=O)c1nnc(C)n1-2]

The title compound was prepared according to General Procedure C from JQ1-linker (31.0 mg, 55.7 μmol, 1.00 equiv), WcQ (19.3 mg, 61.3 μmol, 1.10 equiv), N,N-diisopropylethyl amine (49 μL, 279 μmol, 5.00 equiv), and HATU (23.3 mg, 61.3 μmol, 1.10 equiv) in dry DMF (1.17 mL, 0.048 M). After purification by column chromatography (ISCO, 4 g column, 0-10% MeOH/CH₂Cl₂, 45 min gradient), the title compound, JQ1-WcQ, was obtained as a white solid (7.5 mg, 8.80 μmol, 16% yield).

¹H NMR (500 MHZ, CD₃OD) δ 7.61 (d, J=7.8 Hz, 1H), 7.44 (d, J=8.6 Hz, 2H), 7.39 (d, J=8.8 Hz, 2H), 7.30 (d, J=8.1 Hz, 1H), 7.15 (s, 1H), 7.09-7.03 (m, 1H), 7.02-6.96 (m, 1H), 4.76 (dd, J=8.7, 5.2 Hz, 1H), 4.67-4.55 (m, 2H), 3.41 (dd, J=14.9, 9.0 Hz, 1H), 3.28-3.23 (m, 2H), 3.13 (dd, J=14.8, 8.6 Hz, 1H), 2.77-2.69 (m, 1H), 2.68 (s, 3H), 2.66-2.60 (m, 1H), 2.43 (s, 3H), 2.17-2.06 (m, 3H), 2.03-1.93 (m, 1H), 1.67 (s, 3H), 1.59-1.51 (m, 2H), 1.49-1.41 (m, 2H), 1.36-1.10 (m, 10H). ¹³C NMR (101 MHZ, CD₃OD) δ 177.5, 176.2, 174.5, 173.3, 172.7, 166.2, 157.0, 152.2, 138.1, 138.0, 138.0, 133.5, 133.3, 132.0, 132.0, 131.3, 129.8, 128.9, 124.6, 122.4, 119.8, 119.3, 112.3, 110.9, 55.6, 55.3, 53.2, 40.5, 38.8, 36.8, 32.4, 30.5, 30.3, 30.1, 30.0, 28.8, 28.4, 27.9, 26.7, 14.4, 12.9, 11.6. IR (ATR-FTIR) 3283 (br), 2926 (m), 1735 (w), 1647 (s), 1534 (m), 1488 (w), 1419 (m), 1265 (w), 1088 (m), 1014 (w), 732 (s), 701 (w). HRMS (ESI) (m/z) [M+H]⁺ calculated for C₄H₅₁ClN₉O₅S, 852.3417. found, 852.3404.

### JQ1-YcQ

The title compound was prepared according to General Procedure C from JQ1-linker (18.1 mg, 32.5 μmol, 1.00 equiv), YcQ (12.8 mg, 39.0 μmol, 1.20 equiv), N,N-diisopropylethyl amine (28 μL, 163 μmol, 5.00 equiv), and HATU (13.6 mg, 35.8 μmol, 1.10 equiv) in dry DMF (0.65 mL, 0.050 M). After purification by column chromatography

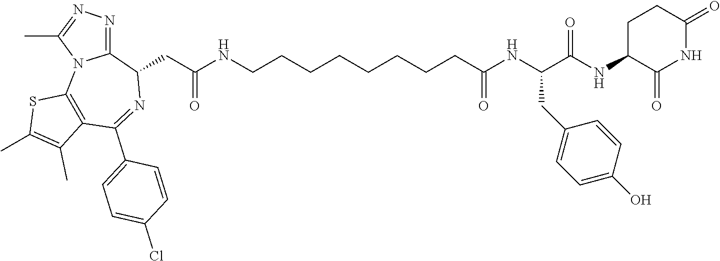 [SMILES: Cc1sc2c(c1C)C(c1ccc(Cl)cc1)=N[C@@H](CC(=O)NCCCCCCCCC(=O)N[C@@H](Cc1ccc(O)cc1)C(=O)N[C@H]1CCC(=O)NC1=O)c1nnc(C)n1-2]

(ISCO, 4 g column, 0-10% MeOH/CH₂Cl₂, 45 min gradient), the title compound, JQ1-YcQ, was obtained as a white solid (17.2 mg, 20.7 μmol, 74% yield).

¹H NMR (400 MHZ, CD₃OD) δ 7.45 (d, J=8.7 Hz, 2H), 7.40 (d, J=8.8 Hz, 2H), 7.08 (d, J=8.5 Hz, 2H), 6.69 (d, J=8.5 Hz, 2H), 4.67-4.61 (m, 2H), 4.61-4.54 (m, 1H), 3.41 (dd, J=14.9, 8.9 Hz, 1H), 3.29-3.20 (m, 2H), 3.12 (dd, J=14.1, 4.9 Hz, 1H), 2.85-2.71 (m, 2H), 2.69 (s, 3H), 2.68-2.60 (m, 1H), 2.44 (s, 3H), 2.24-2.10 (m, 3H), 2.07-1.95 (m, 1H), 1.70 (s, 3H), 1.60-1.52 (m, 2H), 1.52-1.43 (m, 2H), 1.40-1.31 (m, 4H), 1.27-1.08 (m, 5H). ¹³C NMR (101 MHZ, DMSO-d₆) δ 173.0, 172.1, 172.0, 171.7, 169.3, 163.0, 155.7, 155.1, 149.8, 136.7, 135.2, 132.3, 130.7, 130.1, 130.1, 129.8, 129.6, 128.5, 128.0, 114.8, 54.0, 53.9, 49.0, 38.5, 37.7, 37.0, 35.2, 30.9, 29.3, 28.8, 28.7, 28.5, 26.4, 25.2, 24.2, 14.1, 12.7, 11.3. IR (ATR-FTIR) 3283 (br), 2923 (m), 1647 (s), 1541 (s), 1515 (s), 1419 (w), 1243 (w), 1195 (m), 1089 (m), 1014 (w), 840 (w), 732 (m). HRMS (ESI) (m/z) [M+H]⁺ calculated for C₄₂H₅₀ClN₈O₆S, 829.3257. found, 829.3251.

### JQ1-H(Dnp)cQ

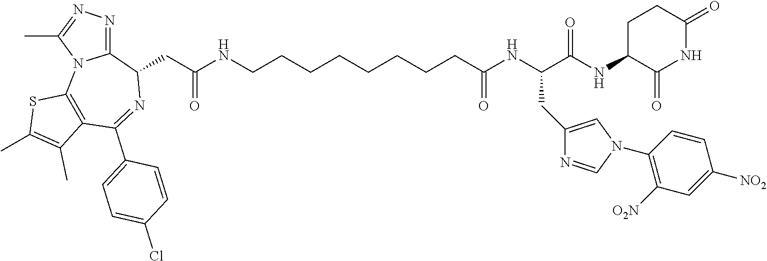 [SMILES: Cc1sc2c(c1C)C(c1ccc(Cl)cc1)=N[C@@H](CC(=O)NCCCCCCCCC(=O)N[C@@H](Cc1cn(-c3ccc([N+](=O)[O-])cc3[N+](=O)[O-])cn1)C(=O)N[C@H]1CCC(=O)NC1=O)c1nnc(C)n1-2]

JQ1-H(Dnp)cQ was prepared according to General Procedure C from JQ1-linker (14.6 mg, 26.3 μmol, 1.00 equiv), H(Dnp)cQ (13.6 mg, 31.5 μmol, 1.20 equiv), N,N-diisopropylethyl amine (23 μL, 131 μmol, 5.00 equiv), and HATU (11.0 mg, 28.9 μmol, 1.10 equiv) in dry DMF (0.55 mL, 0.048 M). After purification by column chromatography (ISCO, 4 g column, 0-10% MeOH/CH₂Cl₂, 45 min gradient), JQ1-H(Dnp)cQ was obtained as a white solid (12.2 mg, 12.6 μmol, 48% yield).

¹H NMR (400 MHZ, CD₃OD) δ 8.90 (d, J=2.6 Hz, 1H), 8.60 (dd, J=8.8, 2.6 Hz, 1H), 8.00-7.86 (m, 2H), 7.44 (d, J=8.6 Hz, 2H), 7.39 (d, J=8.7 Hz, 2H), 7.24 (s, 1H), 4.71 (dd, J=8.2, 6.3 Hz, 1H), 4.66-4.58 (m, 2H), 3.41 (dd, J=14.9, 8.9 Hz, 1H), 3.27-3.15 (m, 3H), 3.00 (dd, J=14.9, 8.2 Hz, 1H), 2.79-2.71 (m, 1H), 2.70 (s, 3H), 2.69-2.62 (m, 1H), 2.45 (s, 3H), 2.28-2.21 (m, 2H), 2.20-2.13 (m, 1H), 2.06-1.94 (m, 1H), 1.70 (s, 3H), 1.62-1.51 (m, 4H), 1.40-1.31 (m, 9H). ¹³C NMR (101 MHZ, DMSO-d₆) δ 173.0, 172.1, 172.1, 171.4, 169.3, 163.0, 155.1, 149.8, 146.1, 143.5, 139.8, 136.8, 136.7, 135.2, 134.6, 132.3, 130.7, 130.1, 129.8, 129.6, 129.3, 128.7, 128.4, 121.3, 116.9, 55.3, 53.9, 52.1, 49.0, 38.5, 37.7, 35.2, 30.9, 29.2, 28.8, 28.7, 28.6, 26.4, 25.1, 24.1, 14.1, 12.7, 11.3. IR (ATR-FTIR) 3340 (br), 2926 (m), 2855 (w), 1725 (m), 1656 (m), 1544 (s), 1384 (m), 1345 (m), 1200 (w), 1089 (m), 1014 (w), 742 (w). HRMS (ESI) (m/z) [M+H]⁺ calculated for C₄₅H₅₀ClN₁₂O₉S, 969.3227. found, 969.3209.

### JQ1-HcQ

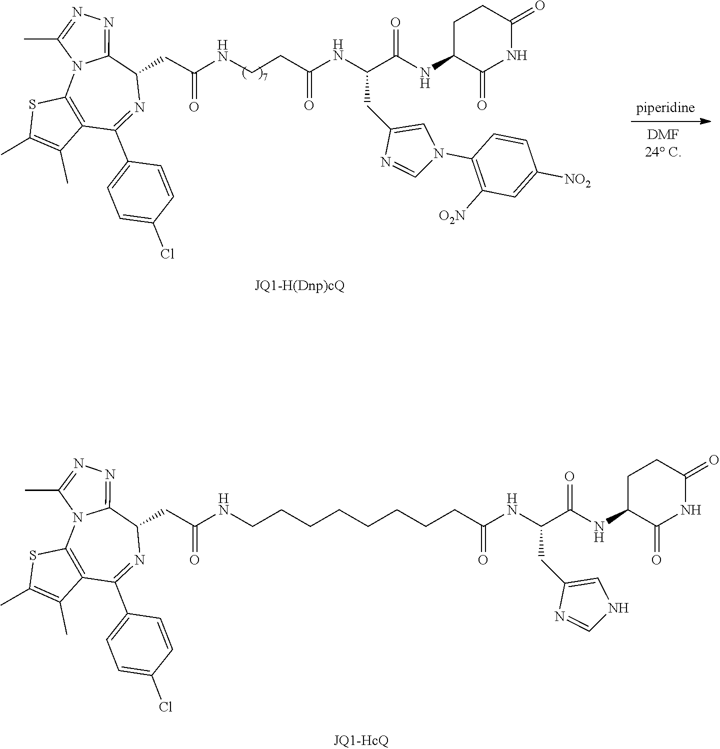

To a solution of JQ1-H(Dnp)cQ (12.2 mg, 12.6 μmol, 1.00 equiv) in DMF (60 μL, 0.21 M) was added piperidine (12 μL, 126 μmol, 10.0 equiv) and stirred at 24° C. for 30 min. The obtained mixture was concentrated with the aid of a rotary evaporator. Purification by preparative HPLC (Waters XBridge Prep C18 OBD, 5 μm, dimensions 19 mm×100 mm, 95-5% MQ water/ACN, 30 min gradient, 280 nm and 254 nm detection, 10 mL/min flow rate) gave the title compound, JQ1-HcQ, as a white solid (2.2 mg, 2.74 μmol, 22% yield).

¹H NMR (400 MHz, CD₃OD) δ 7.61 (br s, 1H), 7.45 (d, J=8.6 Hz, 2H), 7.40 (d, J=8.8 Hz, 2H), 6.91 (d, J=13.3 Hz, 1H), 4.71-4.53 (m, 3H), 3.41 (dd, J=14.9, 8.9 Hz, 1H), 3.28-3.19 (m, 2H), 3.19-3.06 (m, 1H), 3.04-2.83 (m, 1H), 2.81-2.71 (m, 1H), 2.70 (s, 3H), 2.68-2.57 (m, 1H), 2.45 (s, 3H), 2.20 (t, J=7.4 Hz, 2H), 2.14-1.93 (m, 2H), 1.70 (s, 3H), 1.62-1.48 (m, 4H), 1.43-1.26 (m, 9H). ¹³C NMR (101 MHz, DMSO-d₆) δ 172.9, 172.9, 172.0, 171.9, 169.3, 163.0, 155.1, 149.8, 136.7, 135.2, 134.5, 132.3, 130.7, 130.1, 129.8, 129.6, 128.4, 113.0, 53.9, 49.0, 38.5, 37.7, 35.2, 30.8, 30.6, 29.2, 28.8, 28.7, 28.6, 28.5, 26.4, 25.1, 24.1, 14.1, 12.7, 11.3. IR (ATR-FTIR) 3304 (br), 2923 (m), 1653 (s), 1551 (s), 1488 (m), 1387 (m), 1259 (w), 1192 (w), 1088 (m), 1014 (m), 912 (w), 840 (w). HRMS (ESI) (m/z) [M+H]⁺ calculated for C₃₉H₄₈ClN₁₀O₅S, 803.3213. found, 803.3201.

### JQ1-S(TBS)cQ

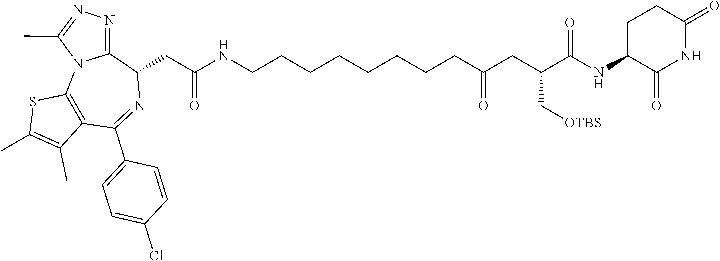 [SMILES: Cc1sc2c(c1C)C(c1ccc(Cl)cc1)=N[C@@H](CC(=O)NCCCCCCCCC(=O)C[C@@H](CO[Si](C)(C)C(C)(C)C)C(=O)N[C@H]1CCC(=O)NC1=O)c1nnc(C)n1-2]

JQ1-S(TBS)cQ was prepared according to General Procedure C from JQ1-linker (10.0 mg, 18.0 mmol, 1.00 equiv), S(TBS)cQ (12.2 mg, 36.9 μmol, 2.05 equiv), N,N-diisopropylethyl amine (15.7 μL, 90.0 μmol, 5.00 equiv), and HATU (7.5 mg, 19.8 μmol, 1.10 equiv) in dry DMF (0.62 mL, 0.048 M). After purification by column chromatography (ISCO, 4 g column, 0-5% MeOH/CH₂Cl₂, 45 min gradient), JQ1-S(TBS)cQ was obtained as a white solid (9.1 mg, 10.5 μmol, 58% yield).

¹H NMR (400 MHZ, CD₃OD) δ 7.46 (d, J=8.8 Hz, 2H), 7.41 (d, J=8.9 Hz, 2H), 4.63 (dd, J=9.0, 5.1 Hz, 1H), 4.58 (dd, J=12.6, 5.2 Hz, 1H), 4.52 (t, J=5.7 Hz, 1H), 3.98-3.80 (m, 2H), 3.41 (dd, J=14.9, 9.0 Hz, 1H), 3.28-3.18 (m, 2H), 2.81-2.71 (m, 1H), 2.70 (s, 3H), 2.69-2.60 (m, 1H), 2.45 (s, 3H), 2.28 (t, J=7.5 Hz, 2H), 2.24-2.14 (m, 1H), 2.07-1.92 (m, 1H), 1.71 (s, 3H), 1.66-1.51 (m, 4H), 1.46-1.31 (m, 9H), 0.90 (s, 9H), 0.09 (s, 6H). 13C NMR (101 MHZ, DMSO-d₆) δ 172.9, 172.2, 171.8, 169.8, 169.3, 163.0, 155.1, 149.8, 136.7, 135.2, 132.3, 130.7, 130.1, 129.8, 129.6, 128.4, 63.4, 54.5, 53.9, 49.2, 38.5, 37.7, 35.2, 30.8, 29.3, 28.9, 28.7, 28.6, 26.4, 25.8, 25.2, 24.1, 18.0, 14.1, 12.7, 11.3, -5.5. IR (ATR-FTIR) 3304 (br), 2927 (m), 2854 (m), 1654 (s), 1541 (s), 1419 (m), 1252 (m), 1195 (m), 1091 (m), 838 (m), 780 (w), 732 (w). HRMS (ESI) (m/z) [M+H]⁺ calculated for C₄₂H₆₀ClN₈O₆SSi, 867.3809. found, 867.3804.

### JQ1-ScQ

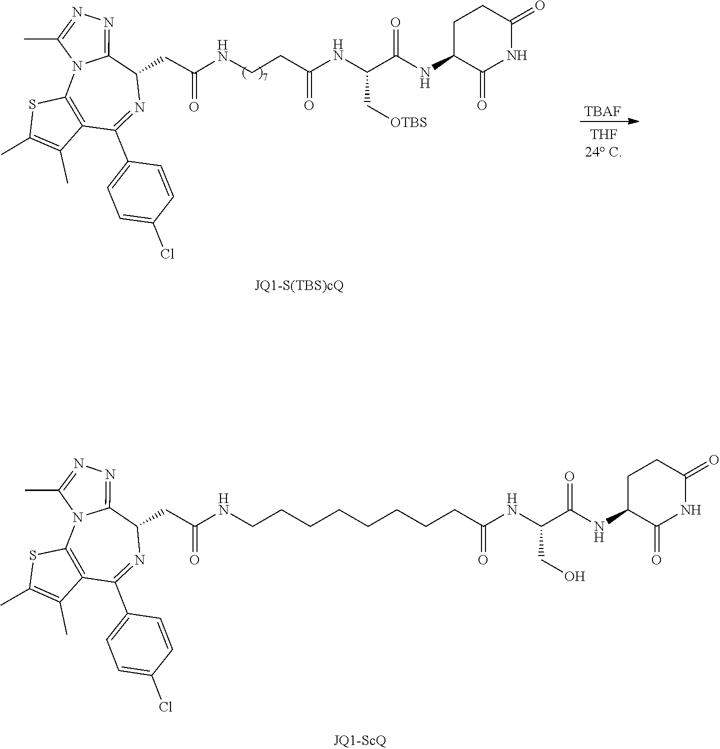

To a solution of JQ1-S(TBS)cQ (10.0 mg, 11.5 μmol, 1.00 equiv) in THF (1.0 mL) was added TBAF (1.0 M in THF, 14 μL, 13.8 μmol, 1.20 equiv) and stirred at 24° C. for 30 min. The obtained mixture was diluted with EtOAc and washed sequentially with brine and saturated aqueous NaHCO₃. The organic layer was dried over Na₂SO₄, filtered, and concentrated with the aid of a rotary evaporator. Purification by preparative HPLC (Waters XBridge Prep C18 OBD, 5 μm, dimensions 19 mm×100 mm, 95-5% MQ water/ACN, 30 min gradient, 280 nm and 254 nm detection, 10 mL/min flow rate) gave the title compound, JQ1-ScQ, as a white solid (2.4 mg, 3.19 μmol, 28% yield).

¹H NMR (400 MHZ, CD₃OD) δ 7.46 (d, J=8.7 Hz, 2H), 7.41 (d, J=8.8 Hz, 2H), 4.67-4.57 (m, 2H), 4.51-4.42 (m, 1H), 3.83-3.75 (m, 2H), 3.41 (dd, J=14.9, 8.9 Hz, 1H), 3.28-3.21 (m, 2H), 2.81-2.71 (m, 1H), 2.70 (s, 3H), 2.68-2.60 (m, 1H), 2.45 (s, 3H), 2.28 (t, J=7.8 Hz, 2H), 2.24-2.14 (m, 1H), 2.08-1.88 (m, 1H), 1.71 (s, 3H), 1.67-1.51 (m, 4H), 1.46-1.26 (m, 9H). ¹³C NMR (101 MHZ, DMSO-d₆) δ 172.9, 172.3, 172.1, 170.3, 169.3, 163.0, 155.1, 149.8, 136.7, 135.2, 132.3, 130.7, 130.1, 129.8, 129.6, 128.5, 61.9, 55.0, 53.9, 49.2, 38.5, 37.7, 35.2, 30.8, 29.3, 28.9, 28.7, 28.7, 26.4, 25.2, 24.1, 14.1, 12.7, 11.3. IR (ATR-FTIR) 3308 (br), 2916 (s), 2850 (s), 1735 (m), 1654 (m), 1590 (m), 1559 (m), 1466 (m), 1378 (m), 1197 (m), 1180 (m), 722 (w). HRMS (ESI) (m/z) [M+H]⁺ calculated for C₃₆H₄₆ClN₈O₆S, 753.2944. found, 753.2942.

### JQ1-T(TBS)cQ

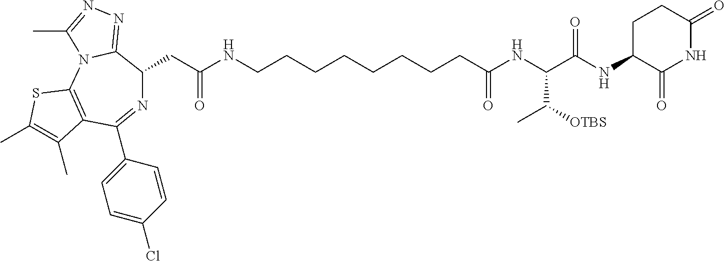 [SMILES: Cc1sc2c(c1C)C(c1ccc(Cl)cc1)=N[C@@H](CC(=O)NCCCCCCCCC(=O)N[C@H](C(=O)N[C@H]1CCC(=O)NC1=O)[C@@H](C)O[Si](C)(C)C(C)(C)C)c1nnc(C)n1-2]

JQ1-T(TBS)cQ was prepared according to General Procedure C from JQ1-linker (10.0 mg, 18.0 μmol, 1.00 equiv), T(TBS)cQ (12.7 mg, 37.0 μmol, 2.00 equiv), N,N-diisopropylethyl amine (15.7 μL, 90.0 μmol, 5.00 equiv), and HATU (7.5 mg, 19.8 μmol, 1.10 equiv) in dry DMF (0.36 mL, 0.050 M). After purification by column chromatography (ISCO, 4 g column, 0-10% MeOH/CH₂Cl₂, 45 min gradient), JQ1-T(TBS)cQ was obtained as a white solid (14.3 mg, 16.2 μmol, 90% yield).

¹H NMR (400 MHZ, CD₃OD) δ 7.46 (d, J=8.6 Hz, 2H), 7.40 (d, J=8.8 Hz, 2H), 4.67-4.63 (m, 1H), 4.62 (dd, J=5.2, 3.1 Hz, 1H), 4.46 (d, J=3.6 Hz, 1H), 4.38-4.29 (m, 1H), 3.41 (dd, J=14.9, 9.0 Hz, 1H), 3.28-3.19 (m, 2H), 2.81-2.71 (m, 1H), 2.70 (s, 3H), 2.68-2.62 (m, 1H), 2.45 (s, 3H), 2.37-2.30 (m, 2H), 2.27-2.20 (m, 1H), 2.01-1.87 (m, 1H), 1.71 (s, 3H), 1.67-1.60 (m, 2H), 1.60-1.54 (m, 2H), 1.42-1.33 (m, 9H), 1.21 (d, J=6.3 Hz, 3H), 0.90 (s, 9H), 0.12 (s, 3H), 0.10 (s, 3H). ¹³C NMR (101 MHZ, Acetone-d₆) δ 173.4, 172.6, 172.4, 171.0, 170.5, 164.1, 156.5, 150.5, 138.3, 136.7, 133.7, 131.6, 131.2, 131.1, 131.1, 129.3, 69.4, 58.9, 55.3, 51.2, 39.7, 39.3, 36.5, 31.8, 30.5, 30.0, 29.8, 29.7, 27.4, 26.3, 26.3, 25.9, 20.0, 18.6, 14.6, 13.0, 11.8, −4.4, −4.7. IR (ATR-FTIR) 3306 (br), 2927 (m), 2855 (m), 1711 (m), 1654 (s), 1534 (m), 1419 (m), 1253 (m), 1195 (m), 1091 (m), 838 (m), 779 (w). HRMS (ESI) (m/z) [M+H]⁺ calculated for C₄₃H₆₂ClN₈O₆SSi, 881.3965. found, 881.3956.

### JQ1-TcQ

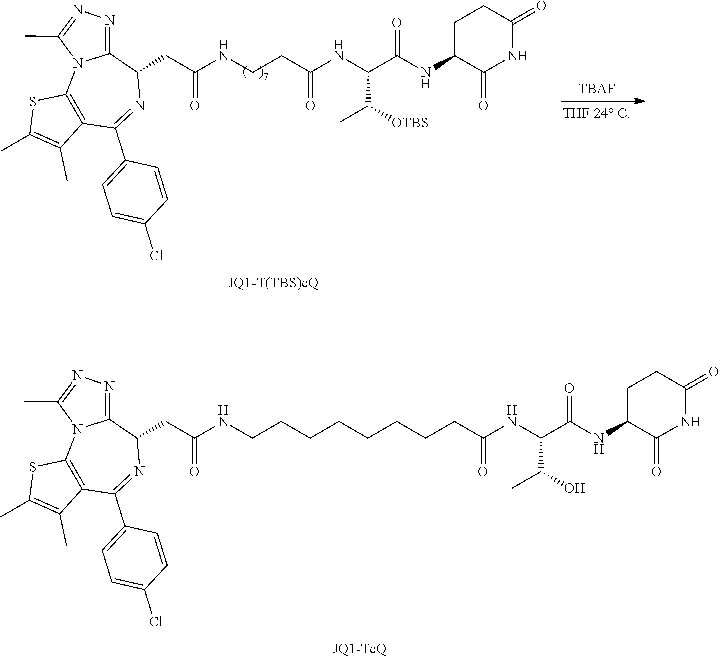

To a solution of JQ1-T(TBS)cQ (12.0 mg, 13.6 μmol, 1.00 equiv) in THF (1.0 mL) was added TBAF (1.0 M in THF, 16 μL, 16.3 μmol, 1.20 equiv). After stirring at 24° C. for 3 h, the obtained mixture was directly purified by preparative HPLC (Waters XBridge Prep C18 OBD, 5 μm, dimensions 19 mm×100 mm, 95-5% MQ water/ACN, 30 min gradient, 280 nm and 254 nm detection, 10 mL/min flow rate) to give the title compound, JQ1-TcQ, as a white solid (8.0 mg, 10.4 μmol, 76% yield).

¹H NMR (400 MHZ, DMSO-d₆) δ 10.81 (br s, 1H), 8.15 (t, J=5.6 Hz, 1H), 8.05 (d, J=7.9 Hz, 1H), 7.65 (d, J=8.7 Hz, 1H), 7.48 (d, J=8.8 Hz, 2H), 7.42 (d, J=8.6 Hz, 2H), 4.75 (d, J=5.4 Hz, 1H), 4.62-4.53 (m, 1H), 4.50 (dd, J=8.1, 6.1 Hz, 1H), 4.23 (dd, J=8.7, 4.3 Hz, 1H), 4.01-3.91 (m, 1H), 3.30-3.01 (m, 5H), 2.77-2.65 (m, 1H), 2.59 (s, 3H), 2.41 (s, 3H), 2.25-2.10 (m, 2H), 1.98-1.89 (m, 2H), 1.63 (s, 3H), 1.52-1.37 (m, 4H), 1.29-1.21 (m, 8H), 1.07 (d, J=6.3 Hz, 3H). ¹³C NMR (101 MHZ, DMSO-d₆) δ 172.9, 172.4, 172.2, 170.4, 169.3, 163.0, 155.1, 149.8, 136.7, 135.2, 132.3, 130.7, 130.1, 129.8, 129.6, 128.4, 66.7, 58.1, 53.9, 49.2, 38.5, 37.7, 35.2, 30.9, 29.3, 28.8, 28.7, 28.7, 26.4, 25.3, 24.2, 19.8, 14.1, 12.7, 11.3. IR (ATR-FTIR) 3306 (br), 2925 (m), 1654 (s), 1592 (m), 1534 (m), 1419 (m), 1384 (m), 1263 (m), 1207 (w), 1089 (m), 841 (m), 732 (m). HRMS (ESI) (m/z) [M+H]⁺ calculated for C₃₇H₄₈ClN₈₀₆S, 767.3101. found, 767.3092.

### JQ1-N(Trt)cQ

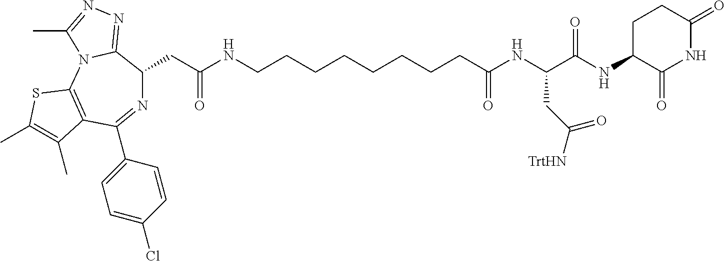

JQ1-N(Trt)cQ was prepared according to General Procedure C from JQ1-linker (18.0 mg, 32.4 μmol, 1.00 equiv), N(Trt)cQ (31.4 mg, 64.7 μmol, 2.00 equiv), N,N-diisopropylethyl amine (28.0 μL, 162 μmol, 5.00 equiv), and HATU (13.6 mg, 35.6 μmol, 1.10 equiv) in dry DMF (1.0 mL, 0.032 M). After purification by column chromatography (ISCO, 4 g column, 0-10% MeOH/CH₂Cl₂, 30 min gradient), JQ1-N(Trt)cQ was obtained as a white solid (14.9 mg, 14.6 μmol, 45% yield).

¹H NMR (400 MHz, CD₃OD) δ 7.45 (d, J=8.5 Hz, 2H), 7.40 (d, J=7.9 Hz, 2H), 7.28-7.18 (m, 15H), 4.79-4.73 (m, 1H), 4.63 (dd, J=8.9, 5.3 Hz, 1H), 4.60-4.51 (m, 1H), 3.40 (dd, J=14.9, 9.0 Hz, 1H), 3.28-3.19 (m, 2H), 2.97-2.79 (m, 2H), 2.79-2.70 (m, 1H), 2.69 (s, 3H), 2.66-2.58 (m, 1H), 2.44 (s, 3H), 2.22 (t, J=7.6 Hz, 2H), 2.14-2.06 (m, 1H), 1.98-1.84 (m, 1H), 1.69 (s, 3H), 1.63-1.50 (m, 4H), 1.44-1.30 (m, 9H). ¹³C NMR (101 MHz, CD₃OD) δ 176.1, 174.8, 173.6, 173.2, 172.6, 171.6, 166.2, 157.0, 152.2, 145.9, 138.1, 138.0, 133.5, 133.3, 132.0, 132.0, 131.3, 130.1, 129.8, 128.7, 127.8, 71.8, 55.3, 51.7, 51.3, 40.5, 39.6, 38.8, 36.9, 32.0, 30.4, 30.3, 30.2, 30.1, 27.9, 26.7, 25.4, 14.4, 12.9, 11.6. IR (ATR-FTIR) 3306 (br), 2927 (m), 1654 (s), 1534 (m), 1447 (m), 1263 (w), 1200 (m), 1091 (w), 843 (s), 736 (w), 701 (m), 558 (m). HRMS (ESI) (m/z) [M+H]⁺ calculated for C₅₆H₆₁ClN₉O₆S, 1022.4149. found, 1022.4139.

### JQ1-NcQ

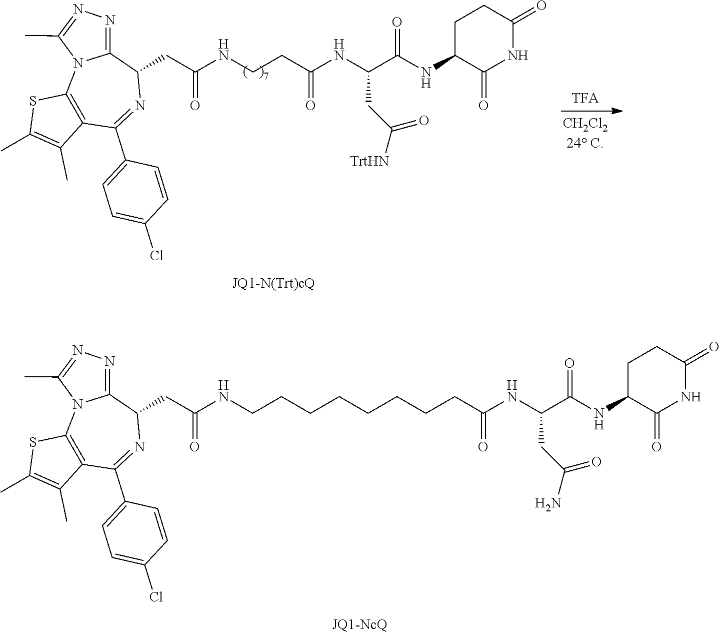

JQ1-N(Trt)cQ (14.9 mg, 14.6 μmol, 1.00 equiv) was dissolved in TFA (0.10 mL) and CH₂Cl₂ (1.0 mL). After stirring at 24° C. for 3 h, the obtained mixture was concentrated with the aid of a rotary evaporator and dried under high vacuum. Purification by preparative HPLC (Waters XBridge Prep C18 OBD, 5 μm, dimensions 19 mm×100 mm, 95-5% MQ water/ACN, 30 min gradient, 280 nm and 254 nm detection, 10 mL/min flow rate) gave the title compound, JQ1-NcQ, as a white solid (4.6 mg, 5.9 μmol, 40% yield).

¹H NMR (400 MHZ, DMSO-d₆) δ 10.79 (br s, 1H), 8.15 (t, J=5.6 Hz, 1H), 8.05 (d, J=8.1 Hz, 1H), 7.96 (t, J=8.7 Hz, 1H), 7.48 (d, J=8.9 Hz, 2H), 7.42 (d, J=8.8 Hz, 2H), 7.25 (br s, 1H), 6.86 (br s, 1H), 4.64-4.55 (m, 1H), 4.55-4.42 (m, 2H), 3.27-3.15 (m, 2H), 3.14-3.01 (m, 2H), 2.78-2.64 (m, 1H), 2.59 (s, 3H), 2.56-2.51 (m, 1H), 2.46-2.30 (m, 1H), 2.41 (s, 3H), 2.15-2.03 (m, 2H), 1.97-1.84 (m, 2H), 1.63 (s, 3H), 1.54-1.37 (m, 4H), 1.33-1.15 (m, 9H). ¹³C NMR (101 MHZ, DMSO-d₆) δ 172.9, 172.1, 172.1, 171.5, 171.3, 169.3, 163.0, 155.1, 149.8, 136.7, 135.2, 132.3, 130.7, 130.1, 129.8, 129.6, 128.4, 53.9, 49.5, 49.1, 38.5, 37.7, 37.4, 35.2, 30.8, 29.3, 28.9, 28.7, 28.6, 26.4, 25.1, 24.1, 14.1, 12.7, 11.3. IR (ATR-FTIR) 3306 (br), 2918 (s), 2850 (m), 1654 (s), 1541 (m), 1446 (m), 1419 (m), 1379 (m), 1255 (w), 1198 (m), 1091 (w), 702 (m). HRMS (ESI) (m/z) [M+H]⁺ calculated for C₃₇H₄₇ClN₉O₆S, 780.3053. found, 780.3041.

### JQ1-Q(Trt)cQ

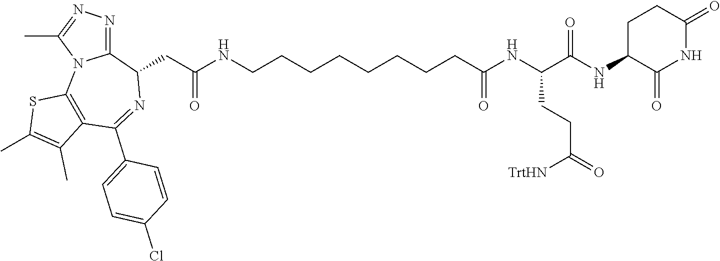

JQ1-Q(Trt)cQ was prepared according to General Procedure C from JQ1-linker (10.0 mg, 18.0 μmol, 1.00 equiv), Q(Trt)cQ (18.0 mg, 36.1 μmol, 1.10 equiv), N,N-diisopropylethyl amine (15.7 μL, 90.0 μmol, 5.00 equiv), and HATU (7.5 mg, 19.8 μmol, 1.10 equiv) in dry DMF (0.36 mL, 0.050 M). After purification by column chromatography (ISCO, 4 g column, 0-5% MeOH/CH₂Cl₂, 45 min gradient), JQ1-Q(Trt)cQ was obtained as a white solid (16.3 mg, 15.7 μmol, 87% yield).

¹H NMR (400 MHZ, CD₃OD) δ 7.45 (d, J=8.8 Hz, 2H), 7.40 (d, J=8.9 Hz, 2H), 7.28-7.18 (m, 15H), 4.67-4.56 (m, 2H), 4.35 (dd, J=8.2, 5.8 Hz, 1H), 3.40 (dd, J=14.9, 8.9 Hz, 1H), 3.27-3.18 (m, 2H), 2.80-2.70 (m, 1H), 2.68 (s, 3H), 2.67-2.59 (m, 1H), 2.58-2.45 (m, 2H), 2.44 (s, 3H), 2.25-2.18 (m, 2H), 2.16-1.86 (m, 4H), 1.69 (s, 3H), 1.63-1.53 (m, 4H), 1.38-1.31 (m, 9H). ¹³C NMR (101 MHZ, CD₃OD) δ 176.3, 174.8, 174.4, 174.1, 173.3, 172.6, 166.2, 157.1, 152.2, 146.0, 138.1, 138.0, 133.5, 133.3, 132.0, 132.0, 131.3, 130.0, 129.8, 128.7, 127.8, 71.6, 55.3, 54.2, 51.1, 40.5, 38.8, 36.8, 33.8, 32.0, 30.5, 30.3, 30.2, 30.2, 29.0, 27.9, 26.8, 25.5, 14.4, 12.9, 11.6. IR (ATR-FTIR) 3306 (br), 2918 (s), 2851 (m), 1654 (s), 1541 (m), 1375 (m), 1259 (m), 1195 (m), 1091 (m), 844 (m), 804 (w), 701 (m). HRMS (ESI) (m/z) [M+H]⁺ calculated for C₅₇H₆₃ClN₉O₆S, 1036.4305. found, 1036.4295.

### JQ1-QcQ

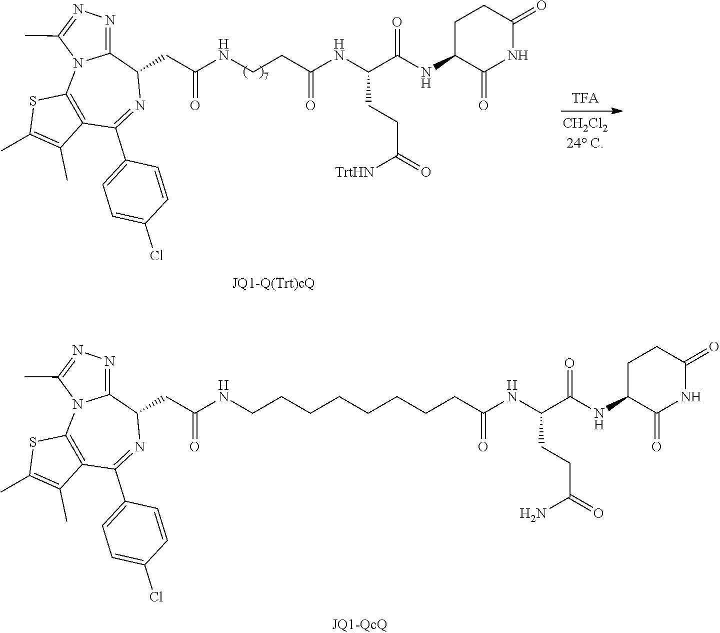

JQ1-Q(Trt)cQ (16.0 mg, 15.4 μmol, 1.00 equiv) was dissolved in TFA (0.10 mL) and CH₂Cl₂ (1.0 mL). After stirring at 24° C. for 2 h, the reaction mixture was concentrated with the aid of a rotary evaporator and dried under high vacuum. Purification by preparative HPLC (Waters XBridge Prep C18 OBD, 5 μm, dimensions 19 mm×100 mm, 95-5% MQ water/ACN, 30 min gradient, 280 nm and 254 nm detection, 10 mL/min flow rate) to give the title compound, JQ1-QcQ, as a white solid (2.3 mg, 2.90 μmol, 19% yield).

¹H NMR (400 MHZ, CD₃OD) δ 7.46 (d, J=8.7 Hz, 2H), 7.41 (d, J=8.8 Hz, 2H), 4.69-4.60 (m, 2H), 4.38 (dd, J=8.1, 6.1 Hz, 1H), 3.41 (dd, J=14.9, 8.9 Hz, 1H), 3.27-3.20 (m, 2H), 2.81-2.71 (m, 1H), 2.70 (s, 3H), 2.69-2.62 (m, 1H), 2.45 (s, 3H), 2.40-2.30 (m, 2H), 2.25 (t, J=7.5 Hz, 2H), 2.19-2.07 (m, 2H), 2.06-1.92 (m, 2H), 1.70 (s, 3H), 1.65-1.53 (m, 4H), 1.44-1.29 (m, 9H). ¹³C NMR (101 MHz, DMSO-d₆) δ 173.8, 173.0, 172.2, 172.1, 171.7, 169.3, 163.0, 155.1, 149.8, 136.7, 135.2, 132.3, 130.7, 130.1, 129.8, 129.6, 128.5, 53.9, 52.0, 49.0, 38.5, 37.7, 35.2, 31.5, 30.9, 29.3, 28.8, 28.7, 28.7, 28.1, 26.4, 25.2, 24.2, 14.1, 12.7, 11.3. IR (ATR-FTIR) 3291 (br), 2918 (m), 2851 (m), 1654 (s), 1592 (m), 1541 (s), 1419 (m), 1378 (m), 1088 (m), 1015 (m), 953 (w), 732 (w). HRMS (ESI) (m/z) [M+H]⁺ calculated for C₃₈H₄₉ClNO₆S, 794.3210. found, 794.3198.

### JQ1-D(Bn)cQ

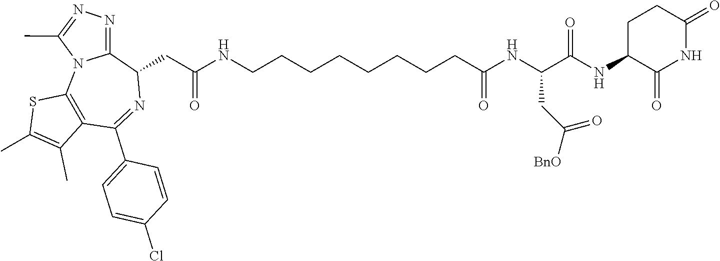 [SMILES: Cc1sc2c(c1C)C(c1ccc(Cl)cc1)=N[C@@H](CC(=O)NCCCCCCCCC(=O)N[C@@H](CC(=O)OCc1ccccc1)C(=O)N[C@H]1CCC(=O)NC1=O)c1nnc(C)n1-2]

JQ1-D(Bn)cQ was prepared according to General Procedure C from JQ1-linker (10.0 mg, 18.0 μmol, 1.00 equiv), D(Bn)cQ (15.0 mg, 45.0 μmol, 1.10 equiv), N,N-diisopropylethyl amine (16 μL, 90.0 mmol, 5.00 equiv), and HATU (7.5 mg, 19.8 μmol, 1.10 equiv) in dry DMF (0.36 mL, 0.050 M). After purification by column chromatography (ISCO, 4 g column, 0-10% MeOH/CH₂Cl₂, 45 min gradient), JQ1-D(Bn)cQ was obtained as a white solid (14.3 mg, 16.4 μmol, 91% yield).

¹H NMR (500 MHZ, CD₃OD) δ 7.45 (d, J=8.7 Hz, 2H), 7.40 (d, J=8.9 Hz, 2H), 7.37-7.28 (m, 5H), 5.13 (s, 2H), 4.84 (dd, J=8.6, 5.3 Hz, 1H), 4.63 (dd, J=9.0, 5.3 Hz, 1H), 4.56 (dd, J=12.7, 5.3 Hz, 1H), 3.41 (dd, J=14.9, 9.0 Hz, 1H), 3.28-3.20 (m, 2H), 2.98-2.91 (m, 1H), 2.81-2.70 (m, 2H), 2.69 (s, 3H), 2.66-2.58 (m, 1H), 2.44 (s, 3H), 2.24-2.16 (m, 2H), 2.15-2.08 (m, 1H), 2.02-1.92 (m, 1H), 1.70 (s, 3H), 1.62-1.51 (m, 4H), 1.39-1.30 (m, 9H). ¹³C NMR (126 MHZ, CD₃OD) δ 176.3, 174.8, 173.1, 173.1, 172.6, 171.7, 166.2, 157.0, 152.2, 138.1, 138.0, 137.4, 133.5, 133.3, 132.0, 132.0, 131.3, 129.8, 129.5, 129.3, 129.3, 67.7, 55.3, 51.3, 40.5, 38.8, 37.3, 36.8, 32.0, 30.5, 30.3, 30.2, 30.1, 27.9, 26.7, 25.4, 14.4, 12.9, 11.6. IR (ATR-FTIR) 3320 (br), 2918 (s), 2850 (m), 1735 (m), 1654 (s), 1541 (m), 1260 (m), 1195 (w), 1091 (m), 1015 (m), 802 (m), 734 (w). HRMS (ESI) (m/z) [M+H]⁺ calculated for C₄₄H₅₂ClN₈O₇S, 871.3363. found, 871.3352.

### JQ1-DcQ

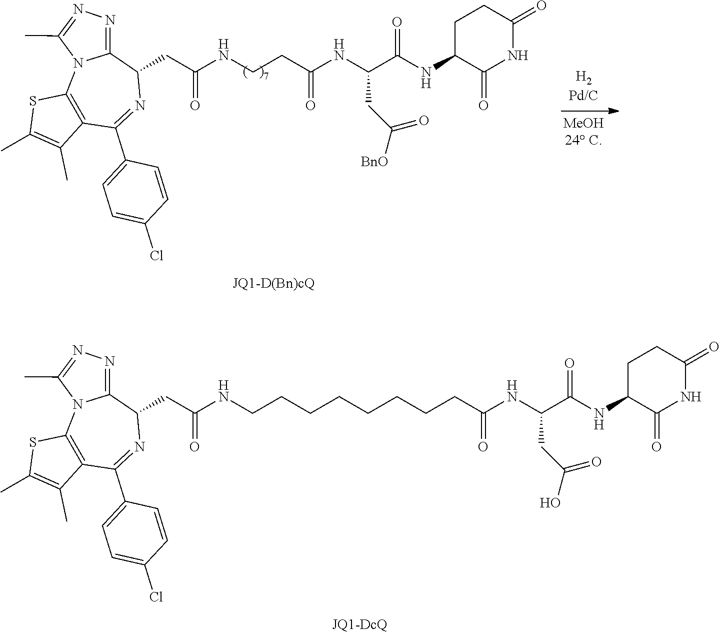

To a solution of JQ1-D(Bn)cQ (10.1 mg, 11.6 μmol, 1.00 equiv) in MeOH (0.58 mL, 20 mM) was added 10% Pd/C (12.3 mg). The reaction mixture was treated with H₂ and stirred at 24° C. for 1 h. After the reaction reached full completion, the obtained mixture was filtered through a short pad of Celite and the filtrate was rinsed with MeOH and then concentrated with the aid of a rotary evaporator. Purification by preparative HPLC (Waters XBridge Prep C18 OBD, 5 μm, dimensions 19 mm×100 mm, 95-5% MQ water/ACN, 30 min gradient, 280 nm and 254 nm detection, 10 mL/min flow rate) gave the title compound, JQ1-DcQ, as a white solid (1.4 mg, 1.79 μmol, 15% yield).

¹H NMR (400 MHZ, DMSO-d₆) δ 12.10 (br s, 1H, OH), 10.78 (br s, 1H, NH), 8.35 (br s, 1H, NH), 8.17 (t, J=5.7 Hz, 1H, NH), 8.06 (d, J=8.4 Hz, 1H, NH), 7.48 (d, J=8.8 Hz, 2H), 7.42 (d, J=8.6 Hz, 2H), 4.64-4.55 (m, 1H), 4.55-4.43 (m, 2H), 3.24-3.14 (m, 2H), 3.14-3.01 (m, 2H), 2.77-2.60 (m, 2H), 2.59 (s, 3H), 2.47-2.42 (m, 1H), 2.41 (s, 3H), 2.08 (t, J=7.4 Hz, 2H), 1.95-1.84 (m, 2H), 1.62 (s, 3H), 1.51-1.38 (m, 4H), 1.31-1.19 (m, 9H). 13C NMR (101 MHz, DMSO-d₆) δ 172.9, 172.0, 169.3, 163.0, 155.1, 149.8, 136.7, 135.2, 132.3, 130.7, 130.1, 129.8, 129.6, 128.4, 53.9, 49.4, 49.1, 38.5, 37.7, 35.3, 30.8, 29.2, 28.8, 28.7, 28.6, 26.4, 25.2, 24.1, 14.1, 12.7, 11.3. IR (ATR-FTIR) 3379 (br), 2925 (m), 2855 (w), 1649 (m), 1590 (m), 1552 (s), 1487 (w), 1382 (s), 1204 (m), 1089 (m), 1013 (m), 912 (w). HRMS (ESI) (m/z) [M+H]⁺ calculated for C₃₇H₄₆ClN₈O₇S, 781.2893. found, 781.2886.

### JQ1-E(Bn)cQ

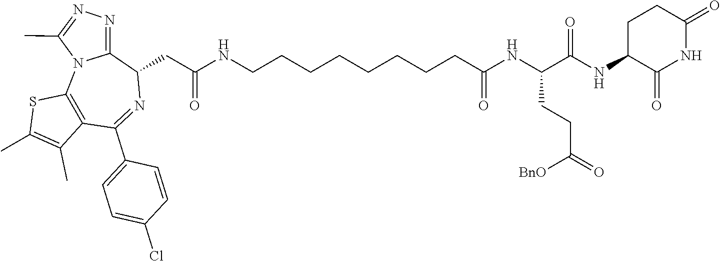 [SMILES: Cc1sc2c(c1C)C(c1ccc(Cl)cc1)=N[C@@H](CC(=O)NCCCCCCCCC(=O)N[C@@H](CCC(=O)OCc1ccccc1)C(=O)N[C@H]1CCC(=O)NC1=O)c1nnc(C)n1-2]

JQ1-E(Bn)cQ was prepared according to General Procedure C from JQ1-linker (10.0 mg, 18.0 μmol, 1.00 equiv), E(Bn)cQ (15.6 mg, 45.0 μmol, 2.50 equiv), N,N-diisopropylethyl amine (16 μL, 90.0 μmol, 5.00 equiv), and HATU (7.5 mg, 19.8 mmol, 1.10 equiv) in dry DMF (0.36 mL, 0.050 M). After purification by column chromatography (ISCO, 4 g column, 0-10% MeOH/CH₂Cl₂, 45 min gradient), JQ1-E(Bn)cQ was obtained as a white solid (10.4 mg, 11.7 μmol, 65% yield).

¹H NMR (400 MHZ, CD₃OD) δ 7.45 (d, J=8.8 Hz, 2H), 7.40 (d, J=8.9 Hz, 2H), 7.38-7.24 (m, 5H), 5.12 (s, 2H), 4.66-4.59 (m, 2H), 4.43 (dd, J=8.5, 5.8 Hz, 1H), 3.41 (dd, J=14.9, 9.0 Hz, 1H), 3.28-3.18 (m, 2H), 2.80-2.70 (m, 1H), 2.69 (s, 3H), 2.68-2.61 (m, 1H), 2.54 (t, J=7.8 Hz, 2H), 2.44 (s, 3H), 2.22 (t, J=7.4 Hz, 2H), 2.19-2.09 (m, 2H), 2.06-1.92 (m, 2H), 1.70 (s, 3H), 1.63-1.51 (m, 4H), 1.42-1.30 (m, 9H). ¹³C NMR (101 MHZ, CD₃OD) δ 176.3, 174.8, 174.3, 173.9, 173.1, 172.7, 166.2, 157.0, 152.2, 138.1, 138.0, 137.6, 133.5, 133.3, 132.0, 132.0, 131.3, 129.8, 129.5, 129.2, 129.2, 67.4, 55.3, 53.9, 51.1, 40.5, 38.8, 36.8, 32.1, 31.3, 30.5, 30.3, 30.2, 30.2, 28.3, 27.9, 26.8, 25.5, 14.4, 12.9, 11.6. IR (ATR-FTIR) 3321 (br), 2916 (s), 2850 (s), 1735 (m), 1654 (m), 1559 (m), 1457 (w), 1260 (w), 1175 (w), 1092 (m), 1032 (m), 800 (m). HRMS (ESI) (m/z) [M+H]⁺ calculated for C₄₅H₅₄ClN₈O₇S, 885.3519. found, 885.3502.

### JQ1-EcQ

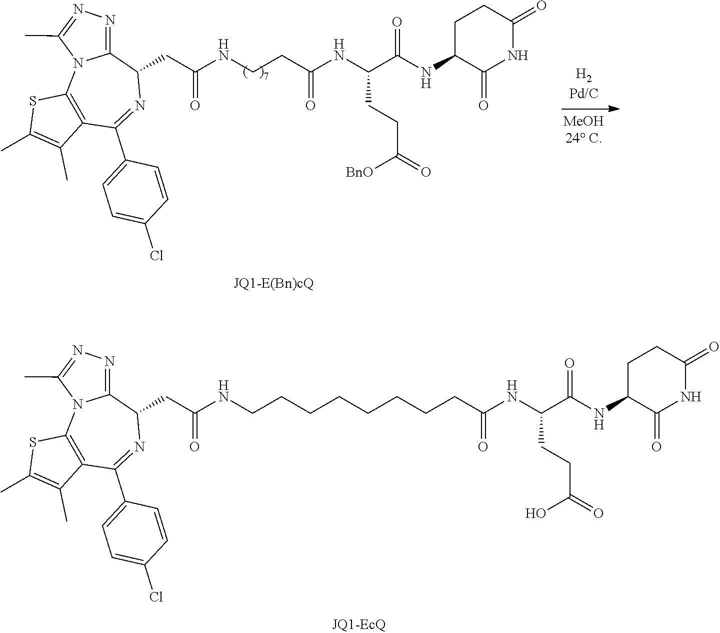

To a solution of JQ1-E(Bn)cQ (6.9 mg, 7.79 μmol, 1.00 equiv) in MeOH (0.39 mL, 20 mM) was added 10% Pd/C (8.2 mg). The reaction mixture was treated with H₂ and stirred at 24° C. for 1 h. After the reaction reached full completion, the obtained mixture was filtered through a short pad of Celite and the filtrate was concentrated with the aid of a rotary evaporator. Purification by preparative HPLC (Waters XBridge Prep C18 OBD, 5 μm, dimensions 19 mm×100 mm, 95-5% MQ water/ACN, 30 min gradient, 280 nm and 254 nm detection, 10 mL/min flow rate) gave the title compound, JQ1-EcQ, as a white solid (3.1 mg, 3.90 μmol, 50% yield).

¹H NMR (400 MHZ, DMSO-d₆) δ 10.77 (br s, 1H, NH), 8.40 (br s, 1H, NH), 8.21 (br s, 2H, NH), 7.48 (d, J=8.8 Hz, 2H), 7.42 (d, J=8.6 Hz, 2H), 4.61-4.43 (m, 2H), 4.29-4.14 (m, 1H), 3.23-3.15 (m, 2H), 3.14-3.00 (m, 2H), 2.79-2.67 (m, 1H), 2.59 (s, 3H), 2.41 (s, 3H), 2.21-2.03 (m, 4H), 2.01-1.80 (m, 3H), 1.77-1.67 (m, 1H), 1.62 (s, 3H), 1.51-1.39 (m, 4H), 1.33-1.19 (m, 9H). ¹³C NMR (101 MHz, DMSO-d₆) δ 173.0, 172.1, 172.1, 172.1, 171.9, 169.3, 163.0, 155.1, 149.8, 136.7, 135.2, 132.2, 130.7, 130.1, 129.8, 129.6, 128.4, 55.3, 53.9, 49.0, 38.5, 37.6, 35.2, 30.9, 29.2, 28.8, 28.7, 28.7, 26.4, 25.2, 24.2, 14.1, 12.7, 11.3. IR (ATR-FTIR) 3296 (br), 2922 (m), 2851 (m), 1647 (s), 1558 (s), 1418 (m), 1260 (m), 1197 (m), 1088 (m), 1014 (w), 731 (m), 700 (w). HRMS (ESI) (m/z) [M+H]⁺ calculated for C₃₈H₄₈ClN₈O₇S, 795.3050. found, 795.3042.

### JQ1-K(2-Cl-Z)cQ

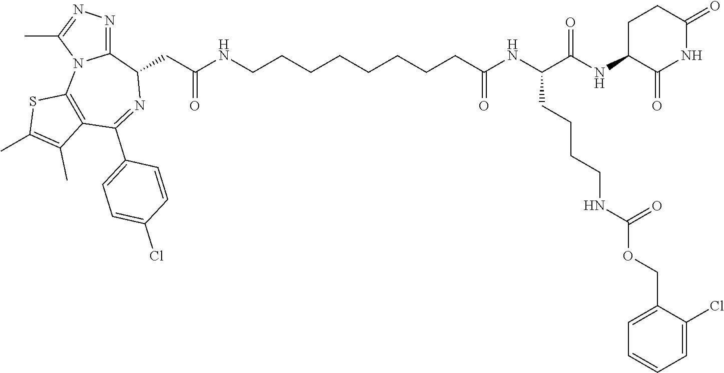 [SMILES: Cc1sc2c(c1C)C(c1ccc(Cl)cc1)=N[C@@H](CC(=O)NCCCCCCCCC(=O)N[C@@H](CCCCNC(=O)OCc1ccccc1Cl)C(=O)N[C@H]1CCC(=O)NC1=O)c1nnc(C)n1-2]

JQ1-K(2-Cl-Z)cQ was prepared according to General Procedure C from JQ1-linker (10.0 mg, 18.0 μmol, 1.00 equiv), K(2-Cl-Z)cQ (16.0 mg, 37.8 μmol, 2.10 equiv), N,N-diisopropylethyl amine (16 μL, 90.0 μmol, 5.00 equiv), and HATU (7.5 mg, 19.8 μmol, 1.10 equiv) in dry DMF (0.36 mL, 0.050 M). After purification by column chromatography (ISCO, 4 g column, 0-10% MeOH/CH₂Cl₂, 30 min gradient), JQ1-K(2-Cl-Z)cQ was obtained as a white solid (9.7 mg, 10.1 μmol, 56% yield).

¹H NMR (400 MHZ, CD₃OD) δ 7.50-7.34 (m, 6H), 7.32-7.26 (m, 2H), 5.16 (s, 2H), 4.68-4.54 (m, 2H), 4.34 (dd, J=8.7, 5.5 Hz, 1H), 3.41 (dd, J=14.9, 9.0 Hz, 1H), 3.29-3.19 (m, 2H), 3.14 (t, J=6.6 Hz, 2H), 2.83-2.70 (m, 1H), 2.69 (s, 3H), 2.67-2.58 (m, 1H), 2.44 (s, 3H), 2.24 (t, J=7.5 Hz, 2H), 2.18-2.07 (m, 1H), 2.07-1.92 (m, 1H), 1.92-1.79 (m, 1H), 1.70 (s, 3H), 1.64-1.43 (m, 8H), 1.40-1.24 (m, 10H). ¹³C NMR (101 MHZ, CD₃OD) δ 176.3, 174.8, 174.7, 173.2, 172.7, 166.2, 158.6, 157.0, 152.2, 138.1, 138.0, 136.0, 134.1, 133.5, 133.3, 132.0, 132.0, 131.4, 130.5, 130.4, 130.3, 129.8, 128.2, 64.6, 55.2, 54.7, 51.0, 41.5, 40.5, 38.8, 36.8, 32.8, 32.0, 30.5, 30.4, 30.3, 30.2, 28.0, 27.9, 26.9, 25.6, 24.0, 14.4, 12.9, 11.6. IR (ATR-FTIR) 3320 (br), 2918 (s), 2850 (m), 1718 (m), 1654 (s), 1559 (m), 1541 (m), 1457 (m), 1256 (m), 1198 (m), 1093 (w), 847 (w). HRMS (ESI) (m/z) [M+H]⁺ calculated for C₄₇H₅₈C₁₂N₉O₇S, 962.3551. found, 962.3544.

### JQ1-KcQ

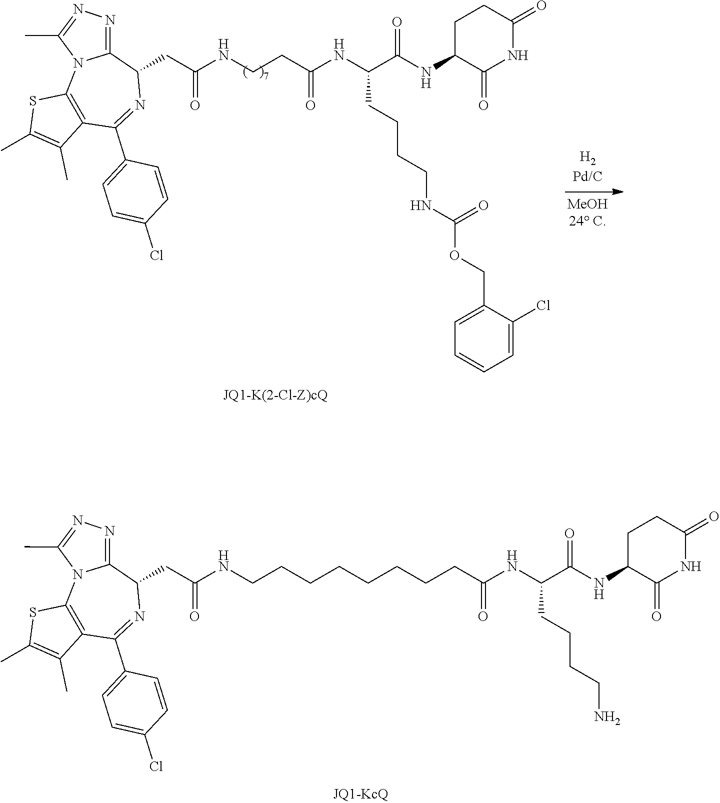

To a solution of JQ1-K(2-Cl-Z)cQ (9.7 mg, 10.1 μmol, 1.00 equiv) in MeOH (0.25 mL, 0.040 M) was added 10% Pd/C (10.7 mg). The reaction mixture was treated with H₂ and stirred at 24° C. for 15 h. After the reaction reached full completion, the obtained mixture was filtered through a short pad of Celite and the filtrate was rinsed with MeOH and then concentrated with the aid of a rotary evaporator. The obtained solid was triturated with diethyl ether (2.0 mL×3) to give the title compound, JQ1-KcQ, as a white solid (6.0 mg, 7.55 μmol, 75% yield).

¹H NMR (400 MHZ, CD₃OD) δ 7.46 (d, J=8.6 Hz, 2H), 7.41 (d, J=8.9 Hz, 2H), 4.70-4.57 (m, 2H), 4.38 (dd, J=7.9, 6.2 Hz, 1H), 3.41 (dd, J=15.0, 9.0 Hz, 1H), 3.27-3.20 (m, 2H), 2.94 (t, J=7.4 Hz, 2H), 2.82-2.72 (m, 1H), 2.71 (s, 3H), 2.69-2.61 (m, 1H), 2.45 (s, 3H), 2.25 (t, J=7.4 Hz, 2H), 2.20-2.08 (m, 1H), 2.08-1.80 (m, 1H), 1.77-1.71 (m, 1H), 1.70 (s, 3H), 1.69-1.66 (m, 1H), 1.66-1.44 (m, 7H), 1.43-1.25 (m, 10H). ¹³C NMR (101 MHZ, CD₃OD) δ 176.3, 174.7, 174.3, 173.4, 172.7, 166.2, 157.0, 152.2, 138.1, 138.0, 133.5, 133.3, 132.0, 132.0, 131.3, 129.8, 55.3, 54.3, 51.0, 40.6, 40.5, 38.8, 36.8, 32.6, 32.1, 30.5, 30.3, 30.2, 30.2, 28.2, 27.9, 26.8, 25.6, 23.6, 14.4, 12.9, 11.6. IR (ATR-FTIR) 3281 (br), 2923 (s), 2854 (m), 1654 (s), 1541 (s), 1419 (m), 1375 (w), 1259 (w), 1198 (m), 1089 (m), 1014 (w), 724 (w). HRMS (ESI) (m/z) [M+H]⁺ calculated for C₃₉H₅₃ClN₉O₅S, 794.3573. found, 794.3573.

### JQ1-R(Pbf)cQ

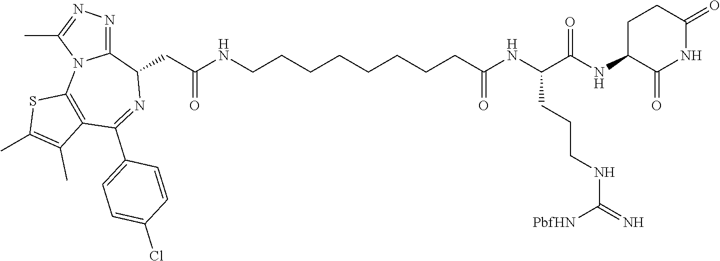

JQ1-R(Pbf)cQ was prepared according to General Procedure C from JQ1-linker (27.8 mg, 50.0 μmol, 1.00 equiv), R(Pbf)cQ (53.6 mg, 100 μmol, 2.00 equiv), N,N-diisopropylethyl amine (44 μL, 250 μmol, 5.00 equiv), and HATU (20.9 mg, 55.0 μmol, 1.10 equiv) in dry DMF (1.00 mL, 0.050 M). After purification by column chromatography (ISCO, 4 g column, 0-10% MeOH/CH₂Cl₂, 45 min gradient), JQ1-R(Pbf)cQ was obtained as a white solid (21.9 mg, 20.4 μmol, 41% yield).

¹H NMR (400 MHZ, CD₃OD) δ 7.45 (d, J=8.7 Hz, 2H), 7.39 (d, J=8.7 Hz, 2H), 4.70-4.54 (m, 2H), 4.44-4.34 (m, 1H), 3.41 (dd, J=15.0, 8.9 Hz, 1H), 3.28-3.12 (m, 4H), 2.99 (s, 2H), 2.80-2.70 (m, 1H), 2.69 (s, 3H), 2.68-2.61 (m, 1H), 2.57 (s, 3H), 2.50 (s, 3H), 2.44 (s, 3H), 2.23 (t, J=7.5 Hz, 2H), 2.18-2.11 (m, 1H), 2.07 (s, 3H), 2.05-1.91 (m, 1H), 1.88-1.76 (m, 1H), 1.70 (s, 3H), 1.66-1.51 (m, 7H), 1.44 (s, 6H), 1.40-1.23 (m, 9H). 13C NMR (101 MHZ, CD₃OD) δ 177.5, 176.2, 174.8, 173.3, 172.7, 166.2, 159.8, 158.1, 157.0, 152.2, 139.4, 138.1, 138.0, 134.4, 133.5, 133.3, 132.0, 132.0, 131.3, 129.8, 126.0, 118.4, 87.7, 55.3, 54.2, 53.2, 51.0, 44.0, 40.5, 38.8, 36.8, 32.4, 32.0, 30.5, 30.3, 30.2, 30.2, 28.7, 28.2, 27.9, 27.9, 26.8, 25.5, 19.6, 18.4, 14.4, 12.9, 12.5, 11.6. IR (ATR-FTIR) 3320 (br), 2926 (m), 1649 (m), 1541 (s), 1419 (w), 1251 (m), 1197 (m), 1089 (s), 841 (s). 732 (s), 661 (w), 558 (m). HRMS (ESI) (m/z) [M+H]⁺ calculated for C₅₂H₆₉ClN₁₁O₈S₂, 1074.4455. found, 1074.4433.

### JQ1-RcQ

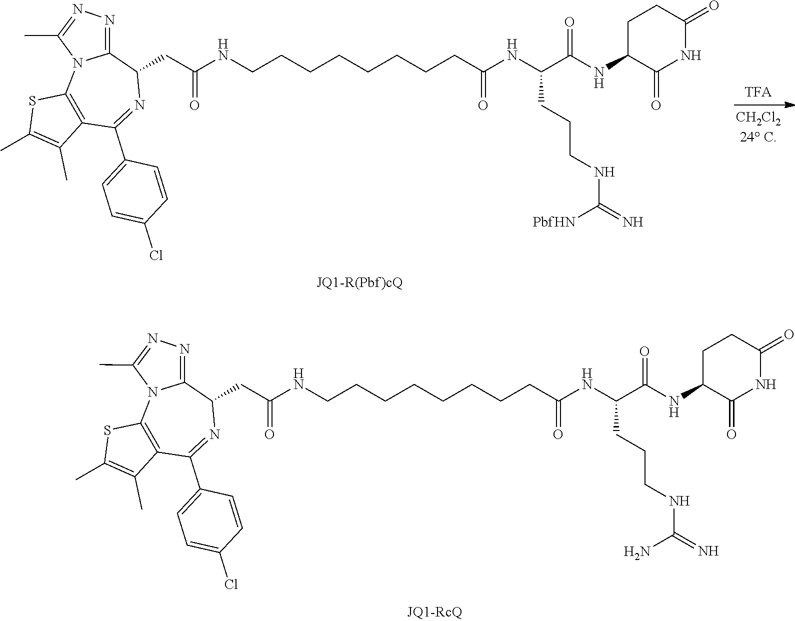

JQ1-R(Pbf)cQ (11.0 mg, 10.2 μmol, 1.00 equiv) was dissolved in TFA (0.95 mL) and CH₂Cl₂ (0.05 mL). After stirring at 24° C. for 2 h, the obtained mixture was concentrated with the aid of a rotary evaporator and dried under high vacuum. Purification by preparative HPLC (Waters XBridge Prep C18 OBD, 5 μm, dimensions 19 mm×100 mm, 95-5% MQ water/ACN, 40 min gradient, 280 nm and 254 nm detection, 10 mL/min flow rate) gave the title compound, JQ1-RcQ as a white solid (4.3 mg, 5.2 μmol, 51% yield).

¹H NMR (500 MHZ, CD₃OD) δ 7.46 (d, J=8.7 Hz, 2H), 7.41 (d, J=8.8 Hz, 2H), 4.63 (dd, J=8.9, 5.6 Hz, 1H), 4.50-4.21 (m, 2H), 3.41 (dd, J=14.7, 9.3 Hz, 1H), 3.29-3.15 (m, 5H), 2.70 (s, 3H), 2.45 (s, 3H), 2.32 (t, J=7.4 Hz, 1H), 2.29-2.24 (m, 2H), 2.22-2.10 (m, 1H), 1.97-1.82 (m, 2H), 1.73 (d, J=7.6 Hz, 1H), 1.70 (s, 3H), 1.69-1.64 (m, 2H), 1.64-1.55 (m, 4H), 1.41-1.32 (m, 9H). ¹³C NMR (101 MHz, DMSO-d₆) δ 173.3, 172.3, 171.9, 169.3, 163.0, 156.9, 155.1, 149.8, 136.7, 135.2, 132.3, 130.7, 130.1, 129.8, 129.6, 128.4, 76.6, 69.8, 55.3, 53.9, 51.6, 40.4, 38.5, 37.7, 35.1, 31.1, 29.2, 28.8, 28.7, 28.7, 26.5, 26.4, 25.3, 24.8, 14.1, 12.7, 11.3. IR (ATR-FTIR) 3291 (br), 2927 (m), 2857 (w), 1654 (s), 1549 (s), 1419 (m), 1381 (m), 1198 (m), 1089 (m), 1014 (m), 841 (w), 804 (w). HRMS (ESI) (m/z) [M+H]⁺ calculated for C₃₉H₅₃ClN₁₁O₅S, 822.3635. found, 822.3623.

### JQ1-C(PMB)cQ

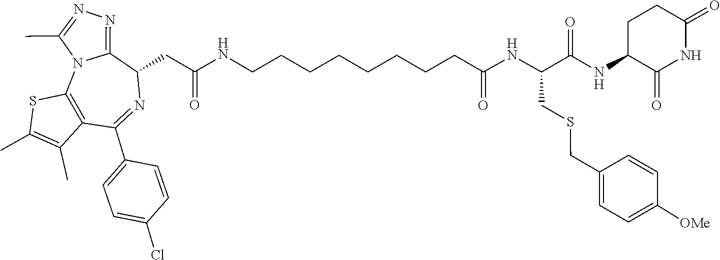 [SMILES: COc1ccc(CSC[C@H](NC(=O)CCCCCCCCNC(=O)C[C@@H]2N=C(c3ccc(Cl)cc3)c3c(sc(C)c3C)-n3c(C)nnc32)C(=O)N[C@H]2CCC(=O)NC2=O)cc1]

JQ1-C(PMB)cQ was prepared according to General Procedure C from JQ1-linker (29.8 mg, 53.6 μmol, 1.00 equiv), C(PMB)cQ (22.6 mg, 64.3 μmol, 1.20 equiv), N,N-diisopropylethyl amine (47 μL, 268 μmol, 5.00 equiv), and HATU (22.4 mg, 58.9 μmol, 1.10 equiv) in dry DMF (1.10 mL, 0.049 M). After purification by column chromatography (ISCO, 4 g column, 0-10% MeOH/CH₂Cl₂, 45 min gradient), JQ1-C(PMB)cQ was obtained as a white solid (12.8 mg, 14.4 μmol, 27% yield).

¹H NMR (400 MHZ, CD₃OD) δ 7.45 (d, J=8.0 Hz, 2H), 7.40 (d, J=8.0 Hz, 2H), 7.25 (d, J=8.3 Hz, 2H), 6.84 (d, J=8.7 Hz, 2H), 4.65-4.57 (m, 3H), 3.76 (s, 3H), 3.73 (s, 2H), 3.41 (dd, J=14.9, 8.9 Hz, 1H), 3.30-3.19 (m, 3H), 2.91 (dd, J=14.0, 5.4 Hz, 1H), 2.80-2.71 (m, 1H), 2.69 (s, 3H), 2.68-2.62 (m, 1H), 2.44 (s, 3H), 2.24 (t, J=7.2 Hz, 2H), 2.20-2.13 (m, 1H), 2.07-1.96 (m, 1H), 1.69 (s, 3H), 1.64-1.53 (m, 4H), 1.39-1.32 (m, 9H). ¹³C NMR (101 MHZ, DMSO-d₆) δ 172.9, 172.3, 171.9, 170.6, 169.3, 163.0, 158.1, 155.1, 149.8, 136.8, 135.3, 132.3, 130.7, 130.2, 130.1, 130.1, 129.8, 129.6, 128.5, 113.7, 55.0, 53.9, 52.0, 49.2, 38.5, 37.7, 35.2, 34.7, 33.4, 30.8, 29.3, 28.9, 28.8, 28.6, 26.4, 25.3, 24.1, 14.1, 12.7, 11.3. IR (ATR-FTIR) 3296 (br), 2925 (m), 1711 (m), 1647 (s), 1539 (s), 1419 (m), 1246 (m), 1195 (m), 1089 (m), 1014 (w), 841 (s), 732 (m). HRMS (ESI) (m/z) [M+H]⁺ calculated for C₄₄H₅₄ClN₈O₆S₂, 889.3291. found, 889.3278.

### JQ1-CcQ

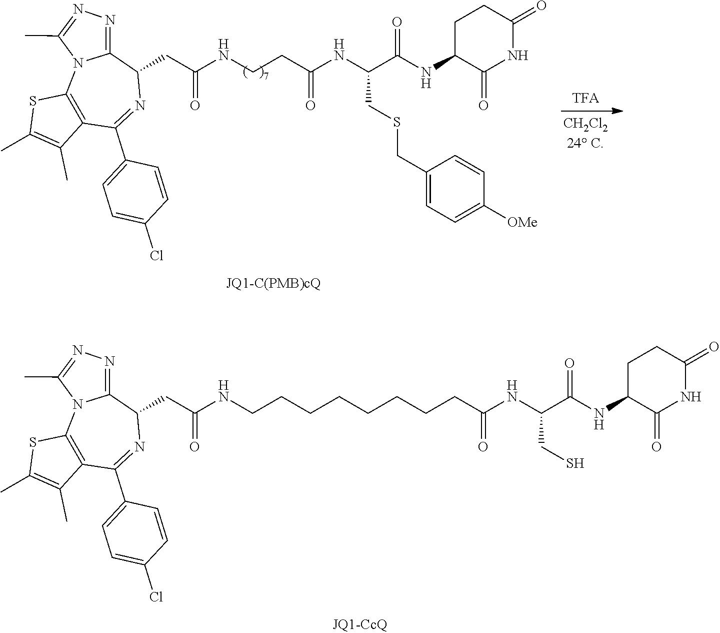

JQ1-C(PMB)cQ (9.1 mg, 10.2 μmol, 1.00 equiv) was dissolved in TFA (0.10 mL) and CH₂Cl₂ (0.10 mL). After stirring at 24° C. for 24 h, the obtained mixture was concentrated with the aid of a rotary evaporator, washed with diethyl ether (2.0 mL×2), and dried under high vacuum. Purification by preparative HPLC (Waters XBridge Prep C18 OBD, 5 μm, dimensions 19 mm×100 mm, 95-5% MQ water/ACN, 30 min gradient, 280 nm and 254 nm detection, 10 mL/min flow rate) gave the title compound, JQ1-CcQ, as a white solid (1.9 mg, 2.41 μmol, 24% yield).

¹H NMR (400 MHZ, DMSO-d₆) δ 10.81 (br s, 1H, NH), 8.28 (d, J=8.1 Hz, 1H, NH), 8.22-8.06 (m, 2H, NH), 7.47 (d, J=8.3 Hz, 2H), 7.42 (d, J=8.3 Hz, 2H), 4.73-4.58 (m, 1H), 4.58-4.43 (m, 2H), 3.24-3.17 (m, 2H), 3.17-3.02 (m, 3H), 2.93-2.81 (m, 1H), 2.78-2.67 (m, 1H), 2.59 (s, 3H), 2.40 (s, 3H), 2.12 (t, J=7.4 Hz, 2H), 2.03-1.88 (m, 2H), 1.62 (s, 3H), 1.50-1.39 (m, 4H), 1.32-1.22 (m, 9H). ¹³C NMR (101 MHZ, DMSO-d₆) δ 172.9, 172.5, 171.8, 170.2, 169.3, 163.0, 155.1, 149.8, 136.7, 135.2, 132.3, 130.7, 130.1, 129.8, 129.6, 128.4, 55.3, 53.9, 51.7, 49.2, 38.5, 37.7, 35.2, 30.8, 29.3, 28.8, 28.7, 28.6, 26.4, 25.1, 24.0, 14.0, 12.7, 11.3. IR (ATR-FTIR) 3320 (br), 2923 (m), 1654 (m), 1551 (s), 1487 (m), 1438 (m), 1387 (m), 1249 (m), 1195 (m), 1089 (m), 1014 (m), 912 (w), 843 (w). LCMS (m/z) [M+H]⁺ calculated for C₃₆H₄₆ClN₈O₅S₂, 769.2716. found, 769.2705.

### epiJQ1-FcQ

The title compound was prepared according to General Procedure C from epiJQ1-linker (11.1 mg, 19.9 μmol, 1.00 equiv), FcQ (12.2 mg, 23.9 μmol, 1.20 equiv), N,N-diisopropylethyl amine (17 μL, 99.8 μmol, 5.00 equiv), and HATU (8.3 mg, 21.9 μmol, 1.10 equiv) in dry DMF (0.45 mL, 0.044 M). After purification by column chromatography

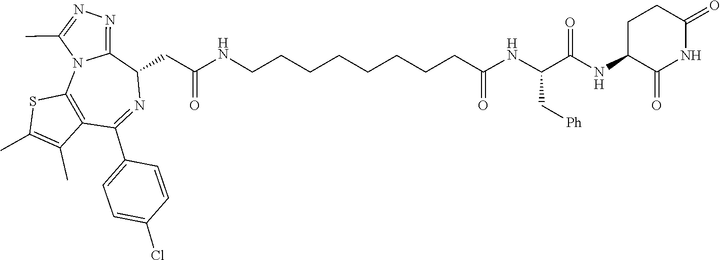 [SMILES: Cc1sc2c(c1C)C(c1ccc(Cl)cc1)=N[C@@H](CC(=O)NCCCCCCCCC(=O)N[C@@H](Cc1ccccc1)C(=O)N[C@H]1CCC(=O)NC1=O)c1nnc(C)n1-2]

(ISCO, 4 g column, 0-10% MeOH/CH₂Cl₂, 45 min gradient), the title compound, epiJQ1-FcQ, was obtained as a white solid (9.9 mg, 12.2 μmol, 61% yield).

¹H NMR (400 MHZ, CD₃OD) δ 7.45 (d, J=8.6 Hz, 2H), 7.40 (d, J=8.7 Hz, 2H), 7.30-7.22 (m, 4H), 7.21-7.15 (m, 1H), 4.71 (dd, J=9.9, 4.8 Hz, 1H), 4.66-4.57 (m, 2H), 3.41 (dd, J=14.9, 8.9 Hz, 1H), 3.29-3.19 (m, 3H), 2.89 (dd, J=14.0, 9.8 Hz, 1H), 2.80-2.70 (m, 1H), 2.69 (s, 3H), 2.68-2.59 (m, 1H), 2.44 (s, 3H), 2.20-2.09 (m, 3H), 2.07-1.96 (m, 1H), 1.69 (s, 3H), 1.61-1.52 (m, 2H), 1.50-1.41 (m, 2H), 1.39-1.21 (m, 9H). ¹³C NMR (101 MHz, DMSO-d₆) δ 173.0, 172.1, 172.0, 171.6, 169.3, 163.0, 155.1, 149.8, 138.0, 136.7, 135.2, 132.3, 130.7, 130.1, 129.8, 129.6, 129.2, 128.5, 127.9, 126.2, 53.9, 53.6, 49.0, 38.5, 37.8, 37.7, 35.2, 30.9, 29.3, 28.8, 28.7, 28.4, 26.4, 25.2, 24.2, 14.1, 12.7, 11.3. IR (ATR-FTIR) 3296 (br), 2925 (s), 2854 (m), 1708 (m), 1647 (s), 1541 (s), 1419 (w), 1249 (w), 1197 (m), 1089 (m), 841 (m), 732 (m). HRMS (ESI) (m/z) [M+H]⁺ calculated for C₄₂H₅₀ClN₈O₅S, 813.3308. found, 813.3293.

### JQ1-epiFcQ

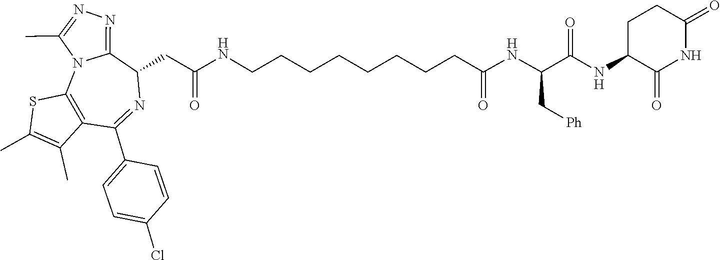 [SMILES: Cc1sc2c(c1C)C(c1ccc(Cl)cc1)=N[C@@H](CC(=O)NCCCCCCCCC(=O)N[C@H](Cc1ccccc1)C(=O)N[C@H]1CCC(=O)NC1=O)c1nnc(C)n1-2]

The title compound was prepared according to General Procedure C from JQ1-linker (11.3 mg, 20.3 μmol, 1.00 equiv), epiFcQ (7.6 mg, 24.4 μmol, 1.20 equiv), N,N-diisopropylethyl amine (18 μL, 102 μmol, 5.00 equiv), and HATU (8.5 mg, 22.4 μmol, 1.10 equiv) in dry DMF (0.46 mL, 0.044 M). After purification by column chromatography (ISCO, 4 g column, 0-10% MeOH/CH₂Cl₂, 45 min gradient), the title compound, JQ1-epiFcQ, was obtained as a white solid (6.8 mg, 8.36 μmol, 41% yield).

¹H NMR (400 MHZ, CD₃OD) δ 7.45 (d, J=8.6 Hz, 2H), 7.40 (d, J=8.8 Hz, 2H), 7.28-7.22 (m, 4H), 7.21-7.15 (m, 1H), 4.71 (dd, J=9.0, 6.2 Hz, 1H), 4.64 (dd, J=9.0, 5.2 Hz, 1H), 4.57 (dd, J=12.4, 5.3 Hz, 1H), 3.42 (dd, J=14.9, 9.0 Hz, 1H), 3.28-3.12 (m, 3H), 2.89 (dd, J=13.8, 9.0 Hz, 1H), 2.75-2.65 (m, 1H), 2.69 (s, 3H), 2.62-2.54 (m, 1H), 2.44 (s, 3H), 2.16 (t, J=7.4 Hz, 2H), 2.05-1.98 (m, 1H), 1.93-1.81 (m, 1H), 1.70 (s, 3H), 1.60-1.52 (m, 2H), 1.52-1.44 (m, 2H), 1.38-1.19 (m, 9H). ¹³C NMR (101 MHZ, CD₃OD) δ 176.1, 174.8, 173.7, 173.0, 172.7, 166.2, 157.1, 152.2, 138.5, 138.1, 138.0, 133.5, 133.3, 132.0, 132.0, 131.3, 130.4, 129.8, 129.4, 127.8, 55.8, 55.3, 51.1, 40.5, 39.1, 38.8, 36.9, 31.9, 30.5, 30.3, 30.2, 30.0, 27.9, 26.8, 25.5, 14.4, 12.9, 11.6. IR (ATR-FTIR) 3296 (br), 2925 (m), 2854 (m), 1707 (m), 1647 (s), 1541 (s), 1419 (m), 1249 (w), 1197 (m), 1089 (m), 841 (w), 701 (w). HRMS (ESI) (m/z) [M+H]⁺ calculated for C₄₂H₅₀ClN₈O₅S, 813.3308. found, 813.3295.

### JQ1-FepicQ

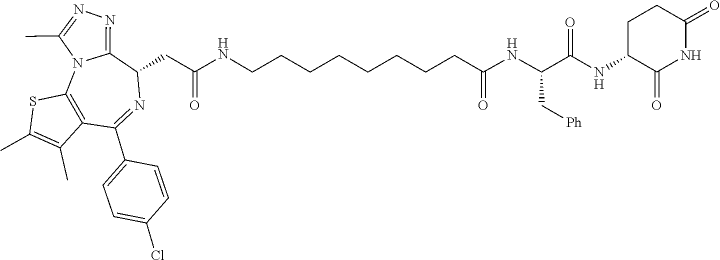 [SMILES: Cc1sc2c(c1C)C(c1ccc(Cl)cc1)=N[C@@H](CC(=O)NCCCCCCCCC(=O)N[C@@H](Cc1ccccc1)C(=O)N[C@@H]1CCC(=O)NC1=O)c1nnc(C)n1-2]

The title compound was prepared according to General Procedure C from JQ1-linker (10.5 mg, 18.9 μmol, 1.00 equiv), FepicQ (7.1 mg, 22.7 μmol, 1.20 equiv), N,N-diisopropylethyl amine (16 μL, 94.4 μmol, 5.00 equiv), and HATU (7.9 mg, 20.8 μmol, 1.10 equiv) in dry DMF (0.38 mL, 0.049 M). After purification by column chromatography (ISCO, 4 g column, 0-10% MeOH/CH₂Cl₂, 45 min gradient), the title compound, JQ1-FepicQ, was obtained as a white solid (2.8 mg, 3.44 μmol, 18% yield).

¹H NMR (500 MHZ, CD₃OD) δ 7.45 (d, J=8.6 Hz, 2H), 7.40 (d, J=8.8 Hz, 2H), 7.30-7.21 (m, 4H), 7.21-7.17 (m, 1H), 4.71 (dd, J=9.0, 6.2 Hz, 1H), 4.63 (dd, J=9.0, 5.2 Hz, 1H), 4.57 (dd, J=12.4, 5.3 Hz, 1H), 3.41 (dd, J=14.9, 9.0 Hz, 1H), 3.28-3.20 (m, 2H), 3.15 (dd, J=13.8, 6.2 Hz, 1H), 2.90 (dd, J=13.7, 9.0 Hz, 1H), 2.76-2.65 (m, 1H), 2.69 (s, 3H), 2.61-2.55 (m, 1H), 2.45 (s, 3H), 2.16 (t, J=7.4 Hz, 2H), 2.04-1.99 (m, 1H), 1.92-1.83 (m, 1H), 1.70 (s, 3H), 1.60-1.53 (m, 2H), 1.51-1.44 (m, 2H), 1.40-1.22 (m, 9H). ¹³C NMR (126 MHZ, CD₃OD) δ 176.1, 174.8, 173.7, 173.0, 172.7, 166.2, 157.1, 152.2, 138.5, 138.1, 138.0, 133.5, 133.3, 132.0, 132.0, 131.3, 130.4, 129.8, 129.4, 127.8, 55.8, 55.3, 51.1, 40.5, 39.1, 38.8, 36.9, 31.9, 30.5, 30.3, 30.2, 30.0, 27.9, 26.8, 25.5, 14.4, 12.9, 11.6. IR (ATR-FTIR) 3291 (br), 2926 (m), 1708 (m), 1647 (s), 1541 (s), 1419 (m), 1364 (w), 1195 (m), 1089 (m), 841 (m), 732 (m), 701 (m). HRMS (ESI) (m/z) [M+H]⁺ calculated for C₄₂H₅₀ClN₈O₅S, 813.3308. found, 813.3293.

### JQ1-cQ

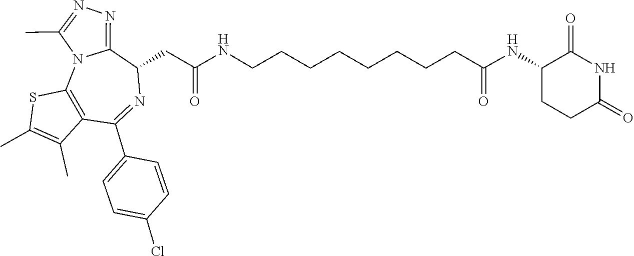 [SMILES: Cc1sc2c(c1C)C(c1ccc(Cl)cc1)=N[C@@H](CC(=O)NCCCCCCCCC(=O)N[C@H]1CCC(=O)NC1=O)c1nnc(C)n1-2]

The title compound was prepared according to General Procedure C from JQ1-linker (16.3 mg, 29.3 μmol, 1.00 equiv), cQ (5.3 mg, 32.2 μmol, 1.10 equiv), N,N-diisopropylethyl amine (25 μL, 146 μmol, 5.00 equiv), and HATU (12.2 mg, 32.2 μmol, 1.10 equiv) in dry DMF (0.62 mL, 0.048 M). After purification by column chromatography (ISCO, 4 g column, 0-10% MeOH/CH₂Cl₂, 45 min gradient), the title compound, JQ1-cQ, was obtained as a white solid (7.4 mg, 11.1 μmol, 38% yield).

¹H NMR (400 MHZ, CD₃OD) δ 7.46 (d, J=8.7 Hz, 2H), 7.41 (d, J=8.7 Hz, 2H), 4.67-4.58 (m, 2H), 3.41 (dd, J=14.9, 9.0 Hz, 1H), 3.30-3.19 (m, 3H), 2.80-2.71 (m, 1H), 2.70 (s, 3H), 2.68-2.60 (m, 1H), 2.45 (s, 3H), 2.30-2.23 (m, 2H), 2.16-2.08 (m, 1H), 2.07-1.94 (m, 1H), 1.71 (s, 3H), 1.67-1.53 (m, 4H), 1.43-1.32 (m, 8H). ¹³C NMR (101 MHZ, DMSO-d₆) δ 173.0, 172.3, 172.1, 169.3, 163.0, 155.1, 149.8, 136.7, 135.2, 132.3, 130.7, 130.1, 129.8, 129.6, 128.4, 53.9, 48.9, 38.5, 37.7, 35.2, 30.9, 29.2, 28.8, 28.7, 28.5, 26.4, 25.2, 24.4, 14.1, 12.7, 11.3. IR (ATR-FTIR) 3294 (br), 2926 (m), 1649 (s), 1531 (s), 1419 (m), 1265 (m), 1194 (m), 1089 (m), 1014 (w), 840 (w), 732 (s), 701 (m). HRMS (ESI) (m/z) [M+H]⁺ calculated for C₃₃H₄₁ClN₇O₄S, 666.2624. found, 666.2618.

### JQ1-CN

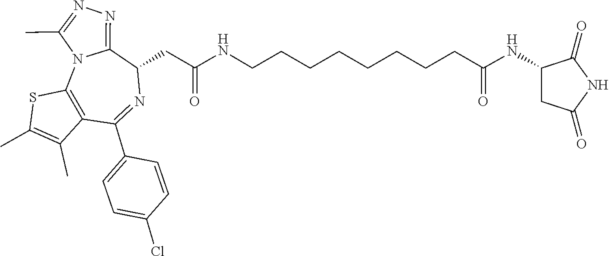 [SMILES: Cc1sc2c(c1C)C(c1ccc(Cl)cc1)=N[C@@H](CC(=O)NCCCCCCCCC(=O)N[C@H]1CC(=O)NC1=O)c1nnc(C)n1-2]

The title compound was prepared according to General Procedure C from JQ1-linker (16.3 mg, 29.3 μmol, 1.00 equiv), cN¹¹⁵ (4.8 mg, 32.2 μmol, 1.10 equiv), N,N-diisopropylethyl amine (25 μL, 146 μmol, 5.00 equiv), and HATU (12.2 mg, 32.2 μmol, 1.10 equiv) in dry DMF (0.62 mL, 0.048 M). After purification by column chromatography (ISCO, 4 g column, 0-10% MeOH/CH₂Cl₂, 45 min gradient), the title compound, JQ1-cN, was obtained as a white solid (6.8 mg, 10.4 μmol, 36% yield).

¹H NMR (400 MHZ, CD₃OD) δ 7.46 (d, J=8.7 Hz, 2H), 7.41 (d, J=8.8 Hz, 2H), 4.63 (dd, J=8.9, 5.3 Hz, 1H), 4.45 (dd, J=9.3, 5.7 Hz, 1H), 3.41 (dd, J=14.9, 9.0 Hz, 1H), 3.29-3.20 (m, 3H), 2.98 (dd, J=17.8, 9.3 Hz, 1H), 2.70 (s, 3H), 2.65 (dd, J=17.8, 5.7 Hz, 1H), 2.45 (s, 3H), 2.21 (t, J=7.5 Hz, 2H), 1.71 (s, 3H), 1.65-1.53 (m, 4H), 1.42-1.30 (m, 8H). ¹³C NMR (101 MHZ, DMSO-d₆) δ 177.7, 176.4, 172.5, 169.3, 163.0, 155.1, 149.8, 136.7, 135.2, 132.3, 130.7, 130.1, 129.8, 129.6, 128.4, 53.9, 49.6, 38.5, 37.7, 36.2, 34.9, 29.2, 28.8, 28.7, 28.5, 26.4, 25.0, 14.1, 12.7, 11.3. IR (ATR-FTIR) 3297 (br), 2926 (m), 1725 (m), 1654 (s), 1551 (s), 1487 (m), 1419 (m), 1191 (w), 1089 (m), 1014 (m), 840 (w), 732 (m). HRMS (ESI) (m/z) [M+H]⁺ calculated for C₃₂H₃₉ClN₇O₄S, 652.2467. found, 652.2460.

### JQ1-FpE

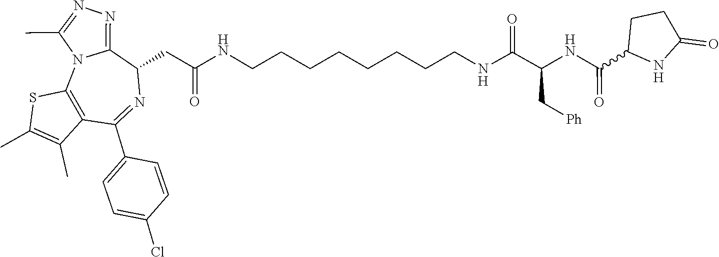 [SMILES: Cc1sc2c(c1C)C(c1ccc(Cl)cc1)=N[C@@H](CC(=O)NCCCCCCCCNC(=O)[C@H](Cc1ccccc1)NC(=O)C1CCC(=O)N1)c1nnc(C)n1-2]

The title compound was prepared according to General Procedure C from JQ1-linker (42.9 mg, 81.5 μmol, 1.00 equiv), FpE (24.8 mg, 89.6 μmol, 1.10 equiv), N,N-diisopropylethyl amine (71 μL, 407 μmol, 5.00 equiv), and HATU (34.1 mg, 89.6 μmol, 1.10 equiv) in dry DMF (1.71 mL, 0.048 M). ¹H NMR analysis of the crude reaction mixture indicated a 6:4 (S)/(R)-pE ratio. After purification by column chromatography (ISCO, 4 g column, 0-10% MeOH/CH₂Cl₂, 45 min gradient), the title compound, JQ1-FpE, was obtained as a white solid (23.1 mg, 29.4 μmol, 36% yield, 6:4 dr).

¹H NMR (major diastereomer, 400 MHz, CD₃OD) δ 7.46 (d, J=8.7 Hz, 2H), 7.40 (d, J=8.8 Hz, 2H), 7.29-7.16 (m, 5H), 4.68-4.56 (m, 2H), 4.19-4.10 (m, 1H), 3.41 (dd, J=14.9, 9.0 Hz, 1H), 3.29-3.20 (m, 2H), 3.19-3.03 (m, 3H), 2.96-2.84 (m, 1H), 2.69 (s, 3H), 2.45 (s, 3H), 2.40-2.17 (m, 3H), 1.92-1.81 (m, 1H), 1.70 (s, 3H), 1.62-1.51 (m, 2H), 1.45-1.17 (m, 11H). ¹³C NMR (major diastereomer, 101 MHz, CD₃OD) δ 181.4, 174.6, 173.0, 172.7, 166.2, 157.0, 152.2, 138.3, 138.1, 138.0, 133.5, 133.3, 132.0, 132.0, 131.3, 130.3, 129.8, 129.5, 127.9, 58.0, 56.0, 55.3, 40.5, 39.4, 39.2, 38.8, 30.5, 30.4, 30.3, 30.3, 30.2, 27.9, 27.8, 26.7, 14.4, 12.9, 11.6. IR (ATR-FTIR) 3290 (br), 2926 (m), 1647 (s), 1531 (m), 1419 (m), 1265 (m), 1089 (m), 1014 (w), 840 (w), 731 (s), 700 (s), 484 (w). HRMS (ESI) (m/z) [M+H]⁺ calculated for C₄₁H₅₀ClN₈O₄S, 785.3359. found, 785.3346.

### JO1-FcN

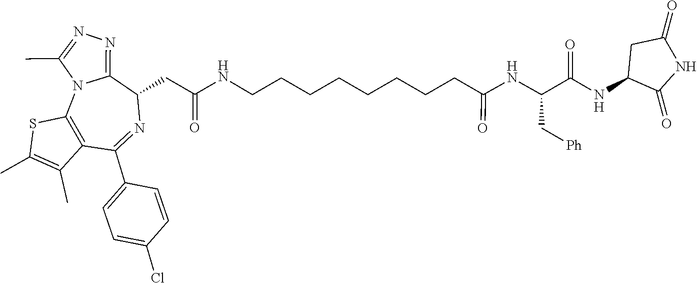 [SMILES: Cc1sc2c(c1C)C(c1ccc(Cl)cc1)=N[C@@H](CC(=O)NCCCCCCCCC(=O)N[C@@H](Cc1ccccc1)C(=O)N[C@H]1CC(=O)NC1=O)c1nnc(C)n1-2]

The title compound was prepared according to General Procedure C from JQ1-linker (30.3 mg, 54.6 μmol, 1.00 equiv), FcN (15.0 mg, 57.3 μmol, 1.05 equiv), N,N-diisopropylethyl amine (48 μL, 273 μmol, 5.00 equiv), and HATU (22.8 mg, 60.0 μmol, 1.10 equiv) in dry DMF (1.15 mL, 0.048 M). After purification by column chromatography (ISCO, 4 g column, 0-10% MeOH/CH₂Cl₂, 45 min gradient), the title compound, JQ1-FcN, was obtained as a white solid (18.4 mg, 23.0 μmol, 42% yield).

¹H NMR (500 MHZ, CD₃OD) δ 7.45 (d, J=8.6 Hz, 2H), 7.40 (d, J=8.8 Hz, 2H), 7.29-7.22 (m, 4H), 7.22-7.17 (m, 1H), 4.70-4.60 (m, 2H), 4.46 (dd, J=9.3, 5.7 Hz, 1H), 3.41 (dd, J=14.9, 9.0 Hz, 1H), 3.29-3.20 (m, 2H), 3.14 (dd, J=13.9, 5.7 Hz, 1H), 2.95-2.84 (m, 2H), 2.69 (s, 3H), 2.58 (dd, J=17.8, 5.7 Hz, 1H), 2.44 (s, 3H), 2.18-2.13 (m, 2H), 1.70 (s, 3H), 1.60-1.52 (m, 2H), 1.50-1.42 (m, 2H), 1.39-1.22 (m, 7H), 1.20-1.12 (m, 2H). ¹³C NMR (101 MHZ, DMSO-d₆) δ 177.7, 176.7, 172.0, 171.5, 169.3, 163.0, 155.1, 149.8, 137.8, 136.7, 135.2, 132.3, 130.7, 130.1, 129.8, 129.6, 129.1, 128.4, 128.0, 126.2, 53.9, 53.3, 49.5, 38.5, 37.7, 37.6, 36.1, 35.2, 29.3, 28.8, 28.7, 28.5, 26.4, 25.1, 14.1, 12.7, 11.3. IR (ATR-FTIR) 3293 (br), 2927 (m), 1721 (s), 1647 (s), 1531 (s), 1419 (m), 1266 (m), 1191 (m), 1089 (m), 1014 (w), 732 (s), 700 (m). HRMS (ESI) (m/z) [M+H]⁺ calculated for C₄₁H₄₈ClN₈O₅S, 799.3151. found, 799.3143.

### JQ1-LcN

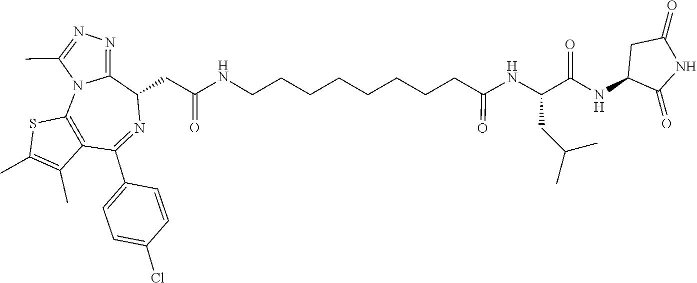 [SMILES: Cc1sc2c(c1C)C(c1ccc(Cl)cc1)=N[C@@H](CC(=O)NCCCCCCCCC(=O)N[C@@H](CC(C)C)C(=O)N[C@H]1CC(=O)NC1=O)c1nnc(C)n1-2]

The title compound was prepared according to General Procedure C from JQ1-linker (16.7 mg, 30.0 μmol, 1.0 equiv), LcN (10.5 mg, 46.0 μmol, 1.53 equiv), N,N-diisopropylethyl amine (26 μL, 150 μmol, 5.00 equiv), and HATU (12.5 mg, 12.5 μmol, 1.10 equiv) in dry DMF (0.63 mL, 0.048 M). After purification by column chromatography (ISCO, 4 g column, 0-10% MeOH/CH₂Cl₂, 45 min gradient), the title compound, JQ1-FcN, was obtained as a white solid (13.1 mg, 17.1 μmol, 57% yield).

¹H NMR (400 MHZ, CD₃OD) δ 7.46 (d, J=8.6 Hz, 2H), 7.40 (d, J=8.8 Hz, 2H), 4.63 (dd, J=9.0, 5.3 Hz, 1H), 4.55 (dd, J=9.3, 5.7 Hz, 1H), 4.43-4.36 (m, 1H), 3.41 (dd, J=14.9, 9.0 Hz, 1H), 3.28-3.19 (m, 2H), 2.98 (dd, J=17.9, 9.4 Hz, 1H), 2.70 (s, 3H), 2.63 (dd, J=17.8, 5.7 Hz, 1H), 2.45 (s, 3H), 2.28-2.21 (m, 2H), 1.70 (s, 3H), 1.67-1.53 (m, 7H), 1.42-1.30 (m, 9H), 0.94 (dd, J=16.0, 6.5 Hz, 6H). ¹³C NMR (101 MHz, DMSO-d₆) δ 177.9, 176.8, 172.5, 172.1, 169.3, 163.0, 155.1, 149.8, 136.7, 135.2, 132.3, 130.7, 130.1, 129.8, 129.6, 128.4, 53.9, 50.4, 49.4, 40.8, 38.5, 37.7, 36.2, 35.2, 29.3, 28.8, 28.8, 28.6, 26.4, 25.2, 24.2, 23.0, 21.4, 14.1, 12.7, 11.3. IR (ATR-FTIR) 3294 (br), 2927 (m), 1725 (m), 1643 (s), 1531 (s), 1419 (m), 1265 (m), 1088 (m), 1014 (w), 840 (w), 731 (s), 701 (m). HRMS (ESI) (m/z) [M+H]⁺ calculated for C₃₈H₅₀ClN₈O₅S, 765.3308. found, 765.3298.

### JO1-HcN

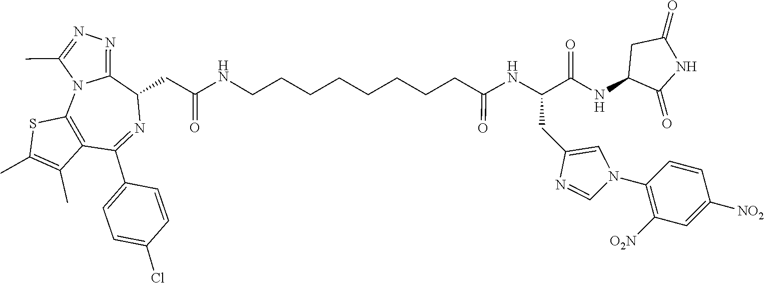 [SMILES: Cc1sc2c(c1C)C(c1ccc(Cl)cc1)=N[C@@H](CC(=O)NCCCCCCCCC(=O)N[C@@H](Cc1cn(-c3ccc([N+](=O)[O-])cc3[N+](=O)[O-])cn1)C(=O)N[C@H]1CC(=O)NC1=O)c1nnc(C)n1-2]

JQ1-H(Dnp)cN was prepared according to General Procedure C from JQ1-linker (30.0 mg, 53.9 μmol, 1.00 equiv), H(Dnp)cN (27.1 mg, 64.7 μmol, 1.20 equiv), N,N-diisopropylethyl amine (47 μL, 270 μmol, 5.00 equiv), and HATU (22.6 mg, 59.3 μmol, 1.10 equiv) in dry DMF (1.1 mL, 0.049 M). After purification by column chromatography (ISCO, 4 g column, 0-10% MeOH/CH₂Cl₂, 45 min gradient), JQ1-H(Dnp)cN was obtained as a white solid (24.9 mg, 26.1 μmol, 44% yield).

¹H NMR (400 MHZ, CD₃OD) δ 8.89 (d, J=2.5 Hz, 1H), 8.61 (dd, J=8.7, 2.6 Hz, 1H), 8.00-7.86 (m, 2H), 7.44 (d, J=8.6 Hz, 2H), 7.38 (d, J=8.7 Hz, 2H), 7.21 (s, 1H), 4.72-4.65 (m, 1H), 4.63 (dd, J=8.9, 5.1 Hz, 1H), 4.54 (dd, J=9.3, 5.7 Hz, 1H), 3.45-3.38 (m, 1H), 3.28-3.20 (m, 2H), 3.16-3.09 (m, 1H), 3.02-2.92 (m, 2H), 2.70 (s, 3H), 2.69-2.62 (m, 1H), 2.45 (s, 3H), 2.28-2.21 (m, 2H), 1.69 (s, 3H), 1.60-1.53 (m, 4H), 1.40-1.30 (m, 9H). ¹³C NMR (101 MHz, CD₃OD) δ 178.8, 178.1, 176.2, 173.7, 172.6, 166.2, 157.0, 152.2, 148.5, 145.7, 140.0, 138.6, 138.1, 138.0, 136.2, 133.5, 133.3, 132.0, 132.0, 131.3, 131.1, 129.8, 129.5, 122.4, 119.5, 55.2, 54.2, 51.4, 40.5, 38.8, 37.1, 36.8, 31.2, 30.4, 30.4, 30.3, 30.2, 27.9, 26.6, 14.4, 12.9, 11.6. IR (ATR-FTIR) 3304 (br), 2919 (m), 2851 (m), 1718 (m), 1654 (m), 1541 (s), 1341 (m), 1260 (m), 1192 (w), 1089 (m), 803 (w), 738 (w). HRMS (ESI) (m/z) [M+H]⁺ calculated for C₄₄H₄₈ClN₁₂O₉S, 955.3071. found, 955.3055.

### JQ1-HcN

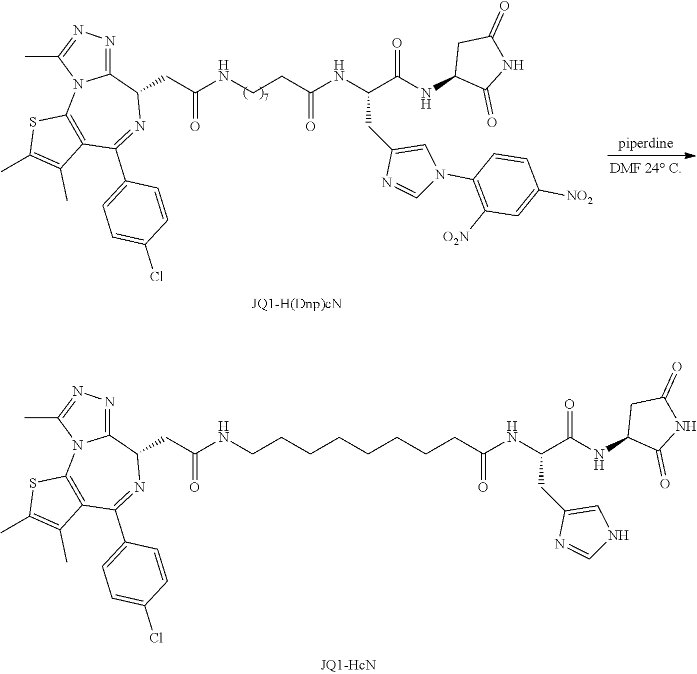

To a solution of JQ1-H(Dnp)cN (11.9 mg, 12.5 μmol, 1.00 equiv) in DMF (60 μL, 0.21 M) was added piperidine (12 μL, 125 μmol, 10.0 equiv). After stirring at 24° C. for 1 h, the obtained mixture was concentrated with the aid of a rotary evaporator. Purification by preparative HPLC (Waters XBridge Prep C18 OBD, 5 μm, dimensions 19 mm×100 mm, 95-5% MQ water/ACN, 30 min gradient, 280 nm and 254 nm detection, 10 mL/min flow rate) gave the title compound, JQ1-HcN, as a white solid (2.5 mg, 3.17 μmol, 25% yield).

¹H NMR (600 MHZ, CD₃OD) δ 7.59 (s, 1H), 7.45 (d, J=8.6 Hz, 2H), 7.41 (d, J=8.6 Hz, 2H), 6.89 (s, 1H), 4.66-4.60 (m, 2H), 4.51 (dd, J=9.3, 5.8 Hz, 1H), 3.41 (dd, J=14.9, 8.6 Hz, 1H), 3.28-3.24 (m, 2H), 3.08 (dd, J=15.4, 5.6 Hz, 1H), 2.99-2.87 (m, 2H), 2.70 (s, 3H), 2.61 (dd, J=17.8, 5.7 Hz, 1H), 2.45 (s, 3H), 2.20 (t, J=7.4 Hz, 2H), 1.70 (s, 3H), 1.59-1.51 (m, 4H), 1.42-1.30 (m, 7H), 1.27-1.23 (m, 2H). ¹³C NMR (101 MHZ, DMSO-d₆) δ 177.5, 176.5, 172.0, 171.6, 169.3, 163.0, 155.1, 149.8, 136.7, 135.2, 134.6, 132.3, 130.7, 130.1, 129.8, 129.6, 128.4, 113.0, 53.9, 52.3, 49.4, 38.5, 37.7, 36.0, 35.3, 30.8, 29.2, 28.8, 28.7, 28.6, 26.4, 25.1, 14.1, 12.7, 11.3. IR (ATR-FTIR) 3267 (br), 2926 (m), 2855 (w), 1719 (s), 1646 (s), 1530 (s), 1418 (m), 1191 (m), 1089 (m), 838 (m), 731 (s), 623 (m). HRMS (ESI) (m/z) [M+H]⁺ calculated for C₃₈H₄₆ClN₁₀O₅S, 789.3056. found, 789.3045.

### General Procedure D: Synthesis of dFKBP-FcQ

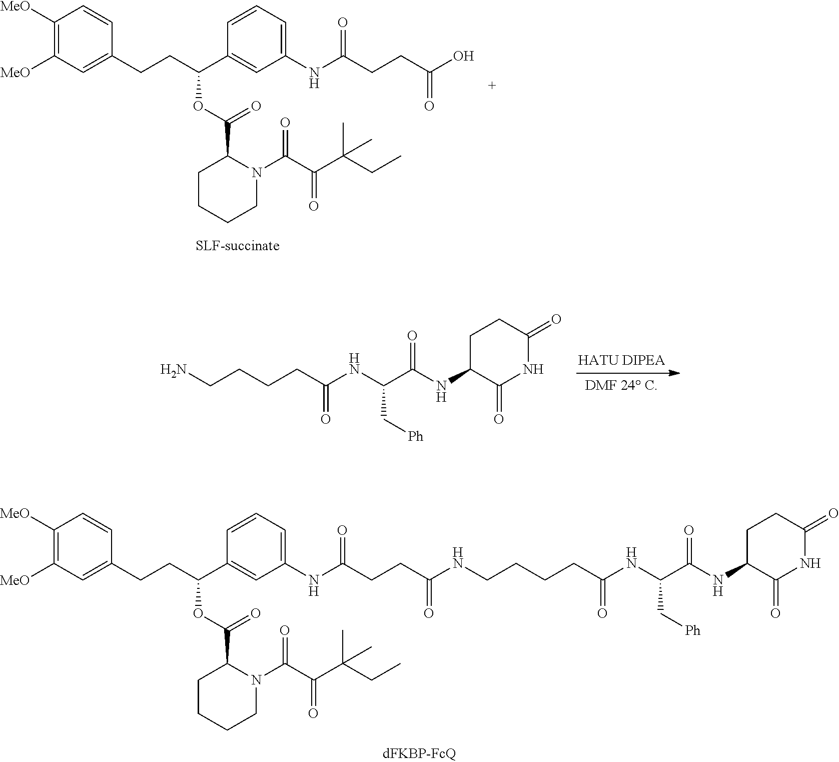 [SMILES: CCC(C)(C)C(=O)C(=O)N1CCCC[C@H]1C(=O)O[C@H](CCc1ccc(OC)c(OC)c1)c1cccc(NC(=O)CCC(=O)O)c1 ; NCCCCC(=O)N[C@@H](Cc1ccccc1)C(=O)N[C@H]1CCC(=O)NC1=O ; CCC(C)(C)C(=O)C(=O)N1CCCC[C@H]1C(=O)O[C@H](CCc1ccc(OC)c(OC)c1)c1cccc(NC(=O)CCC(=O)NCCCCC(=O)N[C@@H](Cc2ccccc2)C(=O)N[C@H]2CCC(=O)NC2=O)c1]

SLF-succinate¹¹³ (7.9 mg, 12.6 μmol, 1.00 equiv) and 5-amino-N—((S)-1-(((S)-2,6-dioxopiperidin-3-yl)amino)-1-oxo-3-phenylpropan-2-yl)pentanamide (6.1 mg, 16.3 μmol, 1.30 equiv) were dissolved in dry DMF (0.13 mL, 0.10 M). N,N-diisopropylethyl amine (11 μL, 62.8 μmol, 5.00 equiv) was then added to the reaction mixture, followed by HATU (5.7 mg, 15.1 μmol, 1.20 equiv). After stirring at 24° C. for 24 h, the reaction mixture was diluted with EtOAc and washed sequentially with brine, 10% aqueous citric acid, and saturated aqueous NaHCO₃. The organic layer was dried over Na₂SO₄, filtered and concentrated with the aid of a rotary evaporator. Purification by column chromatography (ISCO, 4 g column, 0-10% MeOH/CH₂Cl₂, 45 min gradient) gave dFKBP-FcQ as a white solid (8.4 mg, 8.56 μmol, 68% yield).

¹H NMR (major rotamer, 400 MHZ, CD₃OD) δ 7.70 (s, 1H), 7.46-7.38 (m, 1H), 7.31-7.24 (m, 5H), 7.22-7.15 (m, 1H), 7.10-7.03 (m, 1H), 6.85 (d, J=8.3 Hz, 1H), 6.80 (dd, J=7.7, 2.0 Hz, 1H), 6.72 (dd, J=8.1, 2.0 Hz, 1H), 5.77-5.66 (m, 1H), 5.22 (d, J=5.4 Hz, 1H), 4.70 (dd, J=9.9, 4.8 Hz, 1H), 4.60 (dd, J=12.5, 5.4 Hz, 1H), 3.81 (s, 3H), 3.79 (s, 3H), 3.45-3.35 (m, 1H), 3.22 (dd, J=14.0, 4.8 Hz, 1H), 3.15-3.06 (m, 2H), 2.89 (dd, J=14.0, 9.9 Hz, 1H), 2.81-2.69 (m, 1H), 2.69-2.51 (m, 7H), 2.41-2.29 (m, 1H), 2.29-2.21 (m, 1H), 2.21-1.95 (m, 5H), 1.79-1.57 (m, 5H), 1.55-1.40 (m, 3H), 1.39-1.31 (m, 3H), 1.24 (s, 3H), 1.21 (s, 3H), 1.08 (s, 1H), 0.88 (t, J=7.5 Hz, 3H). ¹³C NMR (major rotamer, 101 MHz, CD₃OD) δ 209.1, 175.9, 175.8, 174.5, 174.0, 173.4, 173.0, 171.0, 169.1, 150.4, 148.8, 142.3, 140.4, 138.5, 135.2, 130.3, 130.1, 129.5, 127.8, 123.2, 121.7, 120.6, 119.1, 113.6, 113.3, 78.2, 56.6, 56.5, 56.0, 53.2, 52.8, 47.7, 45.8, 39.9, 39.2, 38.7, 36.1, 33.6, 33.1, 32.3, 31.9, 29.6, 28.3, 27.4, 25.9, 24.0, 23.9, 23.6, 22.1, 9.1. IR (ATR-FTIR) 3291 (br), 2929 (m), 1735 (m), 1640 (s), 1545 (m), 1515 (m), 1443 (m), 1260 (m), 1083 (w), 1028 (w), 734 (m), 701 (m). HRMS (ESI) (m/z) [M+H]⁺ calculated for C₅₃H₆₉N₆O₁₂, 981.4968. found, 981.4959.

### General Procedure: Synthesis of dCDK6-FcQ

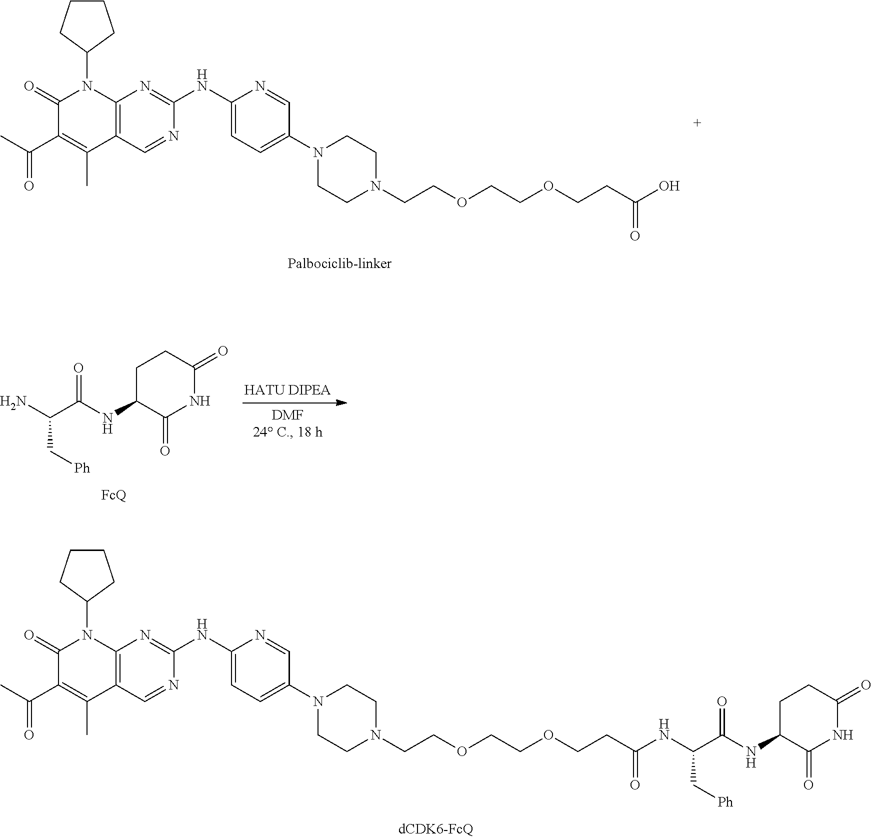 [SMILES: CC(=O)c1c(C)c2cnc(Nc3ccc(N4CCN(CCOCCOCCC(=O)O)CC4)cn3)nc2n(C2CCCC2)c1=O ; N[C@@H](Cc1ccccc1)C(=O)N[C@H]1CCC(=O)NC1=O ; CC(=O)c1c(C)c2cnc(Nc3ccc(N4CCN(CCOCCOCCC(=O)N[C@@H](Cc5ccccc5)C(=O)N[C@H]5CCC(=O)NC5=O)CC4)cn3)nc2n(C2CCCC2)c1=O]

Palbociclib-linker (N. A. Anderson, et al., Bioorg. Med. Chem. Lett. 30, 127106 (2020)) (18.8 mg, 30.9 μmol, 1.00 equiv) and FcQ (10.2 mg, 37.1 μmol, 1.20 equiv) were dissolved in dry DMF (0.77 mL, 0.04 M). N,N-diisopropylethyl amine (27.0 μL, 154 μmol, 5.00 equiv) was then added to the reaction mixture, followed by HATU (14.1 mg, 37.1 μmol, 1.20 equiv). After stirring at 24° C. for 24 h, the reaction mixture was diluted with EtOAc and washed sequentially with brine, 10% aqueous citric acid, and saturated aqueous NaHCO₃. The organic layer was dried over Na₂SO₄, filtered and concentrated with the aid of a rotary evaporator. Purification by preparative HPLC (Agilent 5 Prep-C₁₈ OBD, 5 μm, dimensions 100 mm×30.0 mm, 95-5% MQ water/ACN, 5 min gradient, 250 nm detection, 24 mL/min flow rate) gave dCDK6-FcQ as a yellow solid (16.3 mg, 18.8 μmol, 61% yield).

¹H NMR (400 MHz, DMSO-d₆) δ 10.82 (s, 1H), 10.08 (s, 1H), 8.95 (s, 1H), 8.37 (d, J=8.3 Hz, 1H), 8.13-7.99 (m, 2H), 7.84 (d, J=9.0 Hz, 1H), 7.46 (dd, J=9.1, 3.1 Hz, 1H), 7.30-7.22 (m, 4H), 7.21-7.15 (m, 1H), 5.82 (p, J=8.7 Hz, 1H), 4.63-4.49 (m, 2H), 3.56-3.38 (m, 10H), 3.18-3.11 (m, 3H), 3.08 (dd, J=13.8, 4.3 Hz, 1H), 2.84-2.56 (m, 8H), 2.42 (s, 3H), 2.34-2.28 (m, 2H), 2.31 (s, 3H), 2.27-2.18 (m, 2H), 1.99-1.83 (m, 4H), 1.82-1.72 (m, 2H), 1.64-1.52 (m, 2H). ¹³CNMR (101 MHz, DMSO-d₆) δ 202.5, 173.0, 172.1, 171.3, 169.9, 160.8, 158.6, 158.31, 154.8, 144.3, 143.5, 142.1, 137.9, 135.3, 129.3, 129.2, 128.0, 126.2, 124.7, 115.2, 106.6, 69.6, 69.4, 68.4, 66.7, 57.2, 55.0, 53.6, 52.9, 49.0, 48.3, 37.8, 35.9, 31.3, 30.9, 27.6, 25.1, 24.2, 13.7. IR (ATRFTIR) 2829 (br), 1654 (m), 1579 (s), 1541 (s), 1453 (m), 1290 (m), 1200 (m), 427 (w), 412 (w). HRMS (ESI) (m/z) [M+2H]²⁺ calculated for C₄₅H₅₈N₁₀O₈, 433.2214. found, 433.2218.

### General Procedure E: Synthesis of GGG-FcX

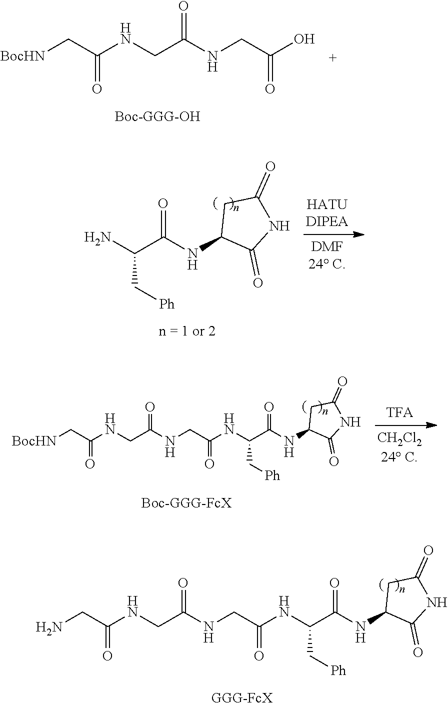

Boc-GGG-OH (28.9 mg, 0.100 mmol, 1.00 equiv) and FcX (0.110 mmol, 1.10 equiv) were dissolved in dry DMF (2.0 mL, 0.050 M). N,N-Diisopropylethyl amine (8.7 μL, 0.500 mmol, 5.00 equiv) and HATU (39.9 mg, 0.105 mmol, 1.05 equiv) were added in sequence to the stirred reaction mixture. After stirring at 24° C. for 18 h, the reaction mixture was concentrated with the aid of a rotary evaporator and the crude material was directly purified by column chromatography (ISCO, 4 g column, 0-20% MeOH/CH₂Cl₂, 40 min gradient). The obtained oil was triturated with diethyl ether (2.0 mL×3) and EtOAc (2.0 mL×3) to give Boc-GGG-FcX (SEQ ID NO: 63).

Boc-GGG-FcX (SEQ ID NO: 63) (1.00 equiv) was dissolved in TFA (0.10 M). After stirring at 24° C. for 1 h, the obtained mixture was concentrated with the aid of a rotary evaporator, washed with diethyl ether (2.0 mL×3), and dried under high vacuum to yield GGG-FcX (SEQ ID NO: 63).

### GGG-FcQ (SEQ ID NO: 8)

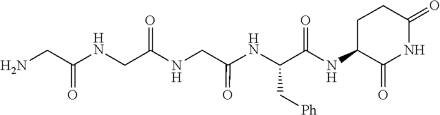 [SMILES: NCC(=O)NCC(=O)NCC(=O)N[C@@H](Cc1ccccc1)C(=O)N[C@H]1CCC(=O)NC1=O]

Boc-GGG-FcQ (SEQ ID NO: 8) was prepared according to General Procedure E from Boc-GGG-OH (28.9 mg, 0.100 mmol, 1.00 equiv), FcQ (30.3 mg, 0.110 mmol, 1.10 equiv), N,N-diisopropylethyl amine (0.087 mL, 0.500 mmol, 5.00 equiv), and HATU (39.9 mg, 0.105 mmol, 1.05 equiv) in dry DMF (2.0 mL, 0.050 M). After purification by column chromatography (ISCO, 4 g column, 5-20% MeOH/CH₂Cl₂, 40 min gradient) as well as trituration with diethyl ether (2.0 mL×3) and EtOAc (2.0 mL×3), Boc-GGG-FcQ (SEQ ID NO: 8) was obtained as a white solid (31.3 mg, 0.0573 mmol, 57% yield).

¹H NMR (500 MHZ, CD₃OD) δ 7.30-7.24 (m, 4H), 7.22-7.12 (m, 1H), 4.65 (dd, J=9.5, 4.7 Hz, 1H), 4.61-4.53 (m, 1H), 3.87-3.70 (m, 6H), 3.24 (dd, J=14.1, 4.7 Hz, 1H), 2.99 (dd, J=14.0, 9.7 Hz, 1H), 2.81-2.57 (m, 2H), 2.15-2.00 (m, 2H), 1.42 (s, 9H). ¹³C NMR (126 MHz, CD₃OD) δ 174.9, 173.6, 173.5, 173.0, 172.6, 171.4, 158.7, 138.6, 130.4, 129.5, 127.7, 81.0, 56.2, 51.3, 44.8, 43.8, 43.5, 38.6, 32.0, 28.7, 25.4. IR (ATR-FTIR) 3283 (br), 2918 (m), 2851 (w), 1701 (s), 1637 (s), 1522 (s), 1508 (s), 1246 (m), 1197 (m), 1166 (m), 1030 (w), 701 (w). HRMS (ESI) (m/z) [M+H]⁺ calculated for C₂₅H₃₅N₆O₈, 547.2511; found, 547.2506.

Boc-GGG-FcQ (SEQ ID NO: 8) (6.6 mg, 12.1 μmol, 1.00 equiv) was dissolved in TFA (0.12 mL, 0.10 M). After stirring at 24° C. for 1 h, the obtained mixture was concentrated with the aid of a rotary evaporator, washed with diethyl ether (2.0 mL×3), and dried under high vacuum to yield the title compound, GGG-FcQ (SEQ ID NO: 8), as a transparent oil (6.8 mg, 12.1 μmol, quantitative).

¹H NMR (500 MHZ, CD₃OD) δ 7.32-7.24 (m, 4H), 7.23-7.17 (m, 1H), 4.70 (dd, J=9.6, 4.8 Hz, 1H), 4.59 (dd, J=10.8, 7.0 Hz, 1H), 3.98-3.87 (m, 3H), 3.81-3.69 (m, 3H), 3.24 (dd, J=13.9, 4.9 Hz, 1H), 3.01 (dd, J=14.0, 9.5 Hz, 1H), 2.82-2.63 (m, 2H), 2.14-2.01 (m, 2H). ¹³C NMR (126 MHZ, CD₃OD) δ 174.8, 173.8, 173.2, 171.8, 171.2, 168.5, 138.4, 130.4, 129.5, 127.8, 55.9, 51.2, 43.8, 43.3, 41.5, 38.9, 32.0, 25.3. IR (ATR-FTIR) 3293 (br), 2927 (m), 2854 (w), 1663 (s), 1549 (s), 1419 (m), 1251 (w), 1202 (s), 1133 (m), 1027 (m), 802 (w), 721 (w). HRMS (ESI) (m/z) [M+H]⁺ calculated for C₂₀H₂₇N₆O₆, 447.1987. found, 447.1982.

### GGG-FcN (SEQ ID NO: 9)

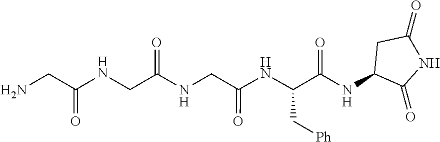 [SMILES: NCC(=O)NCC(=O)NCC(=O)N[C@@H](Cc1ccccc1)C(=O)N[C@H]1CC(=O)NC1=O]

Boc-GGG-FcN (SEQ ID NO: 9) was prepared according to General Procedure E from Boc-GGG-OH (21.1 mg, 0.0729 mmol, 1.00 equiv), FcN (21.0 mg, 0.0802 mmol, 1.10 equiv), N,N-diisopropylethyl amine (0.064 mL, 0.365 mmol, 5.00 equiv), and HATU (30.5 mg, 0.0802 mmol, 1.10 equiv) in dry DMF (1.46 mL, 0.050 M). After purification by column chromatography (ISCO, 4 g column, 5-20% MeOH/CH₂Cl₂, 40 min gradient) as well as trituration with diethyl ether (2.0 mL×3) and EtOAc (1.0 mL×3) analysis (Note: Boc-GGG-FcN is also dissolved in EtOAc and using an excess amount of EtOAc would lead to a lower yield), Boc-GGG-FcN (SEQ ID NO: 9) was obtained as a white solid (24.8 mg, 0.0466 mmol, 64% yield).

¹H NMR (500 MHZ, CD₃OD) & 7.32-7.23 (m, 4H), 7.23-7.16 (m, 1H), 4.60 (dd, J=9.6, 5.3 Hz, 1H), 4.46 (dd, J=9.4, 5.7 Hz, 1H), 3.88-3.71 (m, 6H), 3.22-3.17 (m, 1H), 3.03-2.86 (m, 2H), 2.63 (dd, J=17.9, 5.7 Hz, 1H), 1.43 (s, 9H). ¹³C NMR (126 MHZ, CD₃OD) δ 178.8, 178.1, 173.7, 173.6, 172.8, 171.5, 158.8, 138.5, 130.4, 129.5, 127.8, 81.0, 56.0, 51.6, 44.7, 43.9, 43.6, 38.3, 36.9, 28.7. IR (ATR-FTIR) 3304 (br), 2922 (w), 1654 (s), 1522 (s), 1365 (m), 1248 (m), 1200 (m), 1166 (s), 1133 (m), 1028 (w), 734 (w), 700 (w). HRMS (ESI) (m/z) [M+Na] calculated for C₂₄H₃₂N₆O₈Na, 555.2174. found, 555.2166.

Boc-GGG-FcN (SEQ ID NO: 9) (5.3 mg, 10.0 μmol, 1.00 equiv) was dissolved in TFA (0.50 mL, 0.020 M). After stirring at 24° C. for 1 h, the obtained mixture was concentrated with the aid of a rotary evaporator, washed with diethyl ether (2.0 mL×3), and dried under high vacuum to yield the title compound, GGG-FcN (SEQ ID NO: 9), as a white solid (5.5 mg, 10.0 μmol, quantitative).

¹H NMR (500 MHZ, CD₃OD) δ 7.33-7.24 (m, 4H), 7.23-7.19 (m, 1H), 4.64 (dd, J=9.1, 5.7 Hz, 1H), 4.47 (dd, J=9.4, 5.6 Hz, 1H), 3.99-3.86 (m, 3H), 3.80-3.70 (m, 3H), 3.21-3.14 (m, 1H), 3.03-2.85 (m, 2H), 2.59 (dd, J=17.9, 5.6 Hz, 1H). ¹³C NMR (101 MHZ, DMSO-d₆) δ 177.3, 176.3, 171.0, 168.5, 168.4, 166.6, 137.5, 129.2, 128.1, 126.4, 53.5, 49.5, 48.6, 41.9, 41.7, 37.7, 35.9. IR (ATR-FTIR) 3270 (br), 3066 (w), 1663 (s), 1541 (m), 1426 (m), 1258 (w), 1200 (s), 1132 (s), 836 (w), 800 (m), 721 (m), 640 (w). HRMS (ESI) (m/z) [M+H]⁺ calculated for C₁₉H₂₅N₆O₆, 433.1830. found, 433.1824.

### GGG-FcQ-Me

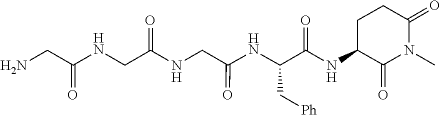 [SMILES: CN1C(=O)CC[C@H](NC(=O)[C@H](Cc2ccccc2)NC(=O)CNC(=O)CNC(=O)CN)C1=O]

Boc-GGG-FcQ-Me (SEQ ID NO: 60) was prepared according to General Procedure E from Boc-GGG-OH (19.8 mg, 69.1 μmol, 1.00 equiv), FcQ-Me (23.8 mg, 82.1 μmol, 1.20 equiv), N,N-diisopropylethyl amine (60 μL, 0.500 μmol, 5.00 equiv), and HATU (28.6 mg, 75.3 μmol, 1.10 equiv) in dry DMF (1.4 mL, 0.049 M). H NMR analysis of the crude reaction mixture indicated a 3:1 (S)/(R)-cQ ratio. After purification by column chromatography (ISCO, 4 g column, 5-20% MeOH/CH₂Cl₂, 40 min gradient) as well as trituration with diethyl ether (2.0 mL×3) and EtOAc (2.0 mL×3), Boc-GGG-FcQ-Me (SEQ ID NO: 60) was obtained as a white solid (18.3 mg, 32.6 μmol, 48% yield, 3:1 dr).

¹H NMR (major diastereomer, 500 MHz, CD₃OD) δ 7.32-7.25 (m, 4H), 7.23-7.18 (m, 1H), 4.69-4.64 (m, 1H), 4.64-4.57 (m, 1H), 3.87-3.71 (m, 6H), 3.28-3.21 (m, 1H), 3.11 (s, 3H), 3.08-2.95 (m, 1H), 2.81-2.77 (m, 2H), 2.14-2.02 (m, 2H), 1.43 (s, 9H). ¹³C NMR (major diastereomer, 101 MHZ, CD₃OD) δ 173.9, 173.6, 173.0, 172.8, 172.6, 171.4, 158.8, 138.6, 130.4, 129.5, 127.8, 80.9, 56.1, 51.9, 44.8, 43.8, 43.5, 38.6, 32.3, 28.7, 27.3, 24.6. IR (ATR-FTIR) 3293 (br), 2978 (w), 2930 (w), 1654 (s), 1528 (s), 1367 (m), 1285 (m), 1249 (m), 1166 (m), 1119 (m), 1030 (m), 701 (m). HRMS (ESI) (m/z) [M+Na] calculated for C₂₆H₃₆N₆O₈Na, 583.2487. found, 583.2481.

Boc-GGG-FcQ-Me (SEQ ID NO: 60) (7.2 mg, 12.8 μmol, 1.00 equiv) was dissolved in TFA (0.13 mL, 0.10 M). After stirring at 24° C. for 1 h, the obtained mixture was concentrated with the aid of a rotary evaporator, washed with diethyl ether (2.0 mL×2), and dried under high vacuum to yield the title compound, GGG-FcQ-Me (SEQ ID NO: 60), as a transparent oil (5.7 mg, 9.92 μmol, 77% yield, 3:1 dr).

¹H NMR (major diastereomer, 400 MHZ, CD₃OD) δ 7.38-7.24 (m, 4H), 7.24-7.17 (m, 1H), 4.73-4.66 (m, 1H), 4.66-4.55 (m, 1H), 4.02-3.85 (m, 3H), 3.81-3.66 (m, 3H), 3.27-3.16 (m, 1H), 3.12 (s, 3H), 3.07-2.96 (m, 1H), 2.87-2.75 (m, 2H), 2.14-1.99 (m, 2H). 13C NMR (major diastereomer, 101 MHz, CD₃OD) δ 173.8, 173.8, 173.7, 173.0, 171.8, 171.2, 138.4, 130.4, 129.5, 127.8, 55.9, 51.8, 43.8, 43.3, 41.6, 38.9, 32.2, 27.3, 24.5. IR (ATR-FTIR) 3291 (br), 3070 (w), 2929 (w), 1664 (s), 1541 (m), 1419 (w), 1202 (m), 1127 (m), 1031 (w), 836 (w), 800 (w), 722 (w). HRMS (ESI) (m/z) [M+H]⁺ calculated for C₂₁H₂₉N₆O₆, 461.2143. found, 461.2140.

### GGG-LcQ

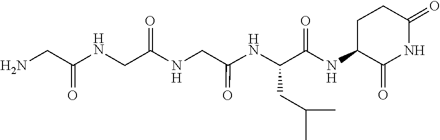 [SMILES: CC(C)C[C@H](NC(=O)CNC(=O)CNC(=O)CN)C(=O)N[C@H]1CCC(=O)NC1=O]

Boc-GGG-LcQ (SEQ ID NO: 33) was prepared according to General Procedure E from Boc-GGG-OH (8.1 mg, 28.1 μmol, 1.00 equiv), LcQ (6.8 mg, 28.1 μmol, 1.00 equiv), N,N-diisopropylethyl amine (24.5 μL, 141 μmol, 5.00 equiv), and HATU (11.2 mg, 29.5 μmol, 1.05 equiv) in dry DMF (0.56 mL, 0.050 M). After purification by preparative HPLC (Agilent 5 Prep-C₁₈ OBD, 5 μm, dimensions 100 mm×30.0 mm, 95-5% MQ water/ACN, 5 min gradient, 220 nm detection, 24 mL/min flow rate), Boc-GGG-LcQ (SEQ ID NO: 33) was obtained as a white solid (4.8 mg, 9.36 μmol, 33% yield).

¹H NMR (400 MHZ, CD₃OD) δ 4.60 (dd, J=11.6, 6.3 Hz, 1H), 4.46 (dd, J=8.8, 6.1 Hz, 1H), 3.96-3.84 (m, 4H), 3.82-3.68 (m, 2H), 2.82-2.62 (m, 2H), 2.15-2.01 (m, 2H), 1.80-1.64 (m, 3H), 1.45 (s, 9H), 0.97 (d, J=6.4 Hz, 3H), 0.93 (d, J=6.3 Hz, 3H).

Boc-GGG-LcQ (SEQ ID NO: 33) (4.8 mg, 9.36 μmol, 1.00 equiv) was dissolved in TFA (0.47 mL) and CH₂Cl₂ (0.47 mL). After stirring at 24° C. for 1 h, the obtained mixture was concentrated with the aid of a rotary evaporator, washed with diethyl ether (2.0 mL×3), and dried under high vacuum to yield the title compound, GGG-LcQ (SEQ ID NO: 33), as a white solid (4.8 mg, 9.12 μmol, 97% yield).

¹H NMR (400 MHZ, DMSO-d₆) δ 10.80 (s, 1H), 8.62 (t, J=5.1 Hz, 1H), 8.30 (d, J=8.4 Hz, 1H), 8.22 (t, J=5.8 Hz, 1H), 8.04 (d, J=8.4 Hz, 1H), 7.99 (s, 3H), 4.59-4.48 (m, 1H), 4.41-4.30 (m, 1H), 3.84 (d, J=5.6 Hz, 2H), 3.80-3.70 (m, 2H), 3.61 (d, J=5.5 Hz, 2H), 2.78-2.65 (m, 1H), 2.49-2.45 (m, 1H), 2.02-1.83 (m, 2H), 1.70-1.58 (m, 1H), 1.56-1.43 (m, 2H), 0.89 (d, J=6.6 Hz, 3H), 0.85 (d, J=6.5 Hz, 3H). ¹³C NMR (101 MHZ, DMSO-d₆) δ 173.0, 172.1, 171.9, 168.5, 168.3, 166.3, 55.1, 50.8, 48.9, 41.9, 41.7, 41.2, 30.9, 24.1, 23.1, 21.6. IR (ATR-FTIR) 3275 (br), 1655 (s), 1541 (m), 1470 (w), 1419 (w), 1363 (m), 1335 (w), 1249 (m), 1199 (s), 1133 (m), 835 (w), 800 (w), 721 (m), 560 (w), 531 (w), 447 (w). HRMS (ESI) (m/z) [M+H]⁺ calculated for C₁₇H₂₉N₆O₆, 413.2143. found, 413.2142.

### GGG-PcQ

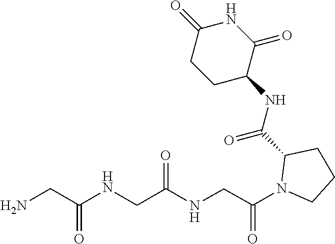 [SMILES: NCC(=O)NCC(=O)NCC(=O)N1CCC[C@H]1C(=O)N[C@H]1CCC(=O)NC1=O]

Boc-GGG-PcQ (SEQ ID NO: 61) was prepared according to General Procedure E from Boc-GGG-OH (10.3 mg, 35.7 μmol, 1.00 equiv), PcQ (8.0 mg, 35.7 μmol, 1.00 equiv), N,N-diisopropylethyl amine (31.1 μL, 179 μmol, 5.00 equiv), and HATU (14.3 mg, 37.5 μmol, 1.05 equiv) in dry DMF (0.71 mL, 0.050 M). After purification by preparative HPLC (Agilent 5 Prep-C18 OBD, 5 μm, dimensions 100 mm×30.0 mm, 95-5% MQ water/ACN, 5 min gradient, 220 nm detection, 24 mL/min flow rate), Boc-GGG-LcQ (SEQ ID NO: 61) was obtained as a white solid (15.1 mg, 30.4 μmol, 85% yield).

¹H NMR (major rotamer, 400 MHZ, CD₃OD) δ 4.61 (dd, J=12.5, 5.4 Hz, 1H), 4.46 (dd, J=8.4, 3.5 Hz, 1H), 4.16-3.83 (m, 4H), 3.75 (s, 2H), 3.72-3.50 (m, 2H), 2.84-2.61 (m, 2H), 2.45-2.16 (m, 2H), 2.14-1.87 (m, 4H), 1.45 (s, 9H).

Boc-GGG-PcQ (SEQ ID NO: 61) (10.0 mg, 20.1 μmol, 1.00 equiv) was dissolved in TFA (0.50 mL) and CH₂Cl₂ (1.00 mL). After stirring at 24° C. for 1 h, the obtained mixture was concentrated with the aid of a rotary evaporator, washed with diethyl ether (2.0 mL×3), and dried under high vacuum to yield the title compound, GGG-PcQ (SEQ ID NO: 61), as a white solid (4.8 mg, 9.12 μmol, 82% yield).

¹H NMR (major rotamer, 400 MHZ, DMSO-d₆) δ 10.81 (s, 1H), 8.62 (t, J=5.8 Hz, 1H), 8.22 (d, J=8.4 Hz, 1H), 8.18-8.10 (m, 1H), 7.98 (s, 3H), 4.61-4.47 (m, 1H), 4.46-4.25 (m, 1H), 4.09-3.90 (m, 2H), 3.89-3.82 (m, 2H), 3.68-3.58 (m, 2H), 3.57-3.33 (m, 2H), 2.79-2.65 (m, 1H), 2.47-2.29 (m, 1H), 2.28-2.00 (m, 2H), 1.95-1.75 (m, 4H). ¹³C NMR (major rotamer, 101 MHz, DMSO-d₆) δ 173.0, 172.1, 171.7, 168.3, 166.7, 166.2, 59.6, 55.1, 49.0, 45.9, 41.8, 41.1, 30.9, 29.4, 24.2, 22.1. IR (ATR-FTIR) 3271 (br), 3085 (br), 1670 (s), 1541 (m), 1438 (w), 1362 (w), 1334 (w), 1250 (w), 1199 (s), 1132 (m). HRMS (ESI) (m/z) [M+H]⁺ calculated for C₁₆H₂₅N₆O₆, 397.1830. found, 397.1846.

### GGG-LcN

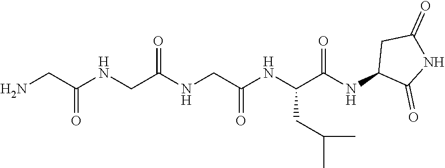 [SMILES: CC(C)C[C@H](NC(=O)CNC(=O)CNC(=O)CN)C(=O)N[C@H]1CC(=O)NC1=O]

Boc-GGG-LcN (SEQ ID NO: 62) was prepared according to General Procedure E from Boc-GGG-OH (12.4 mg, 42.8 μmol, 1.00 equiv), LcN (9.7 mg, 42.8 μmol, 1.00 equiv), N,N-diisopropylethyl amine (37.3 μL, 214 μmol, 5.00 equiv), and HATU (17.1 mg, 44.9 μmol, 1.05 equiv) in dry DMF (0.86 mL, 0.050 M). After purification by preparative HPLC (Agilent 5 Prep-C18 OBD, 5 μm, dimensions 100 mm×30.0 mm, 95-5% MQ water/ACN, 5 min gradient, 220 nm detection, 24 mL/min flow rate), Boc-GGG-LcN (SEQ ID NO: 62) was obtained as a white solid (16.3 mg, 32.7 μmol, 76% yield).

¹H NMR (400 MHZ, CD₃OD) δ 4.52 (dd, J=9.4, 5.6 Hz, 1H), 4.40 (dd, J=10.0, 4.8 Hz, 1H), 3.98-3.82 (m, 4H), 3.82-3.71 (m, 2H), 2.98 (dd, J=17.8, 9.3 Hz, 1H), 2.67 (dd, J=17.9, 5.6 Hz, 1H), 1.77-1.58 (m, 3H), 1.45 (s, 9H), 0.96 (d, J=6.3 Hz, 3H), 0.91 (d, J=6.3 Hz, 3H).

Boc-GGG-LcN (SEQ ID NO: 62) (10.0 mg, 20.1 μmol, 1.00 equiv) was dissolved in TFA (0.50 mL) and CH₂Cl₂ (1.00 mL). After stirring at 24° C. for 1 h, the obtained mixture was concentrated with the aid of a rotary evaporator, washed with diethyl ether (2.0 mL×3), and dried under high vacuum to yield the title compound, GGG-LcN (SEQ ID NO: 62), as a white solid (10.3 mg, 20.1 μmol, quant).

¹H NMR (400 MHZ, DMSO-d₆) δ 11.23 (s, 1H), 8.62 (t, J=5.8 Hz, 1H), 8.54 (d, J=7.9 Hz, 1H), 8.22 (t, J=5.8 Hz, 1H), 8.07 (d, J=8.3 Hz, 1H), 7.99 (s, 3H), 4.53-4.42 (m, 1H), 4.28 (dd, J=8.2, 8.2 Hz, 1H), 3.84 (d, J=5.8 Hz, 2H), 3.78 (t, J=5.9 Hz, 2H), 3.60 (s, 2H), 2.86 (dd, J=17.5, 9.4 Hz, 1H), 2.41 (dd, J=17.5, 5.5 Hz, 1H), 1.64-1.52 (m, 1H), 1.45 (t, J=7.5 Hz, 2H), 0.88 (d, J=6.5 Hz, 3H), 0.83 (d, J=6.5 Hz, 3H). ¹³C NMR (101 MHZ, DMSO-d₆) δ 177.5, 176.4, 172.2, 168.5, 168.5, 166.3, 50.6, 49.3, 41.9, 41.7, 40.9, 36.0, 24.1, 23.0, 21.5. IR (ATR-FTIR) 3272 (br), 3077 (br), 1723 (m), 1659 (s), 1540 (m), 1368 (w), 1244 (w), 1200 (s), 1135 (m), 722 (w). HRMS (ESI) (m/z) [M+H]⁺ calculated for C₁₆H₂₇N₆O₆, 399.1987. found, 399.1983.

### General Procedure F: Synthesis of Fmoc-GGG-FcX

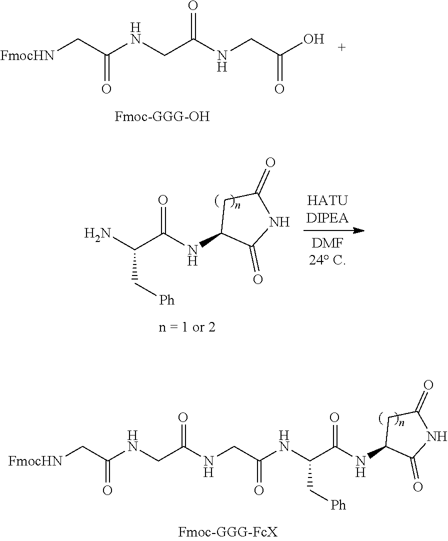

Fmoc-GGG-OH (1.00 equiv) and FcX (1.00 equiv) were dissolved in dry DMF (0.050 M). N,N-diisopropylethyl amine (5.00 equiv) and HATU (1.05 equiv) were added in sequence to the stirred reaction mixture. After stirring at 24° C. for 18 h, the reaction mixture was concentrated with the aid of a rotary evaporator and the crude material was directly purified by column chromatography (ISCO, 4 g column, 5-20% MeOH/CH₂Cl₂, 40 min gradient). The obtained oil was triturated with diethyl ether (2.0 mL×3) and EtOAc (2.0 mL×3) to afford Fmoc-GGG-FcX.

### Fmoc-GGG-FcQ

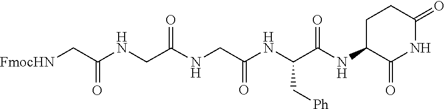

Fmoc-GGG-FcQ (SEQ ID NO: 8) was prepared according to General Procedure F from Fmoc-GGG-OH (49.4 mg, 120 μmol, 1.00 equiv), FcQ (33.0 mg, 120 μmol, 1.00 equiv), N,N-diisopropylethyl amine (0.105 mL, 600 μmol, 5.00 equiv), and HATU (47.9 mg, 126 μmol, 1.05 equiv) in dry DMF (2.4 mL, 0.050 M). After purification by column chromatography (ISCO, 4 g column, 5-20% MeOH/CH₂Cl₂, 40 min gradient) as well as trituration with diethyl ether (2.0 mL×3) and EtOAc (2.0 mL×3), Fmoc-GGG-FcQ (SEQ ID NO: 8) was obtained as a white solid (18.8 mg, 28.1 μmol, 23% yield).

¹H NMR (400 MHZ, DMSO-d₆) δ 10.84 (s, 1H), 8.41 (d, J=8.4 Hz, 1H), 8.19-8.03 (m, 3H), 7.89 (d, J=7.5 Hz, 2H), 7.71 (d, J=7.1 Hz, 2H), 7.57 (t, J=6.1 Hz, 1H), 7.41 (t, J=7.5 Hz, 2H), 7.32 (t, J=6.8 Hz, 2H), 7.28-7.23 (m, 4H), 7.20-7.14 (m, 1H), 4.63-4.49 (m, 2H), 4.32-4.17 (m, 3H), 3.79-3.63 (m, 5H), 3.57 (dd, J=16.8, 5.6 Hz, 1H), 3.16 (d, J=5.3 Hz, 1H), 3.08 (dd, J=13.8, 4.1 Hz, 1H), 2.84-2.69 (m, 2H), 2.04-1.81 (m, 2H). ¹³C NMR (101 MHz, DMSO-d₆) δ 172.9, 171.9, 171.1, 169.5, 169.1, 168.4, 156.5, 143.8, 140.7, 137.8, 129.2, 128.1, 127.6, 127.1, 126.3, 125.2, 120.1, 65.7, 53.8, 49.0, 46.6, 43.5, 42.0, 41.7, 37.7, 30.9, 24.1. IR (ATR-FTIR) 3287 (br), 3066 (w), 2920 (w), 1700 (s), 1660 (s), 1637 (s), 1525 (s), 1450 (m), 1245 (m), 1201 (m), 739 (m), 700 (w). HRMS (ESI) (m/z) [M+Na]⁺ calculated for C₃₅H₃₆N₆O₈Na, 691.2487. found, 691.2469.

### Fmoc-GGG-FcN

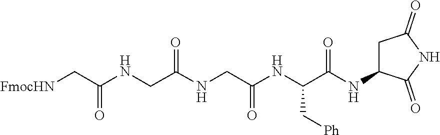

Fmoc-GGG-FcN (SEQ ID NO: 9) was prepared according to General Procedure F from Fmoc-GGG-OH (18.1 mg, 44.0 μmol, 1.00 equiv), FcN (11.5 mg, 44.0 μmol, 1.00 equiv), N,N-diisopropylethyl amine (0.038 mL, 220 μmol, 5.00 equiv), and HATU (17.6 mg, 46.2 μmol, 1.05 equiv) in dry DMF (0.88 mL, 0.050 M). After purification by column chromatography (ISCO, 4 g column, 5-20% MeOH/CH₂Cl₂, 40 min gradient) as well as trituration with diethyl ether (2.0 mL×3) and EtOAc (2.0 mL×3), Fmoc-GGG-FcN (SEQ ID NO: 9) was obtained as a white solid (7.4 mg, 11.3 μmol, 26% yield).

¹H NMR (400 MHZ, DMSO-d₆) δ 11.25 (br s, 1H), 8.63 (d, J=7.8 Hz, 1H), 8.22-8.05 (m, 3H), 7.89 (d, J=7.5 Hz, 2H), 7.71 (d, J=7.4 Hz, 2H), 7.58 (t, J=6.2 Hz, 1H), 7.41 (t, J=7.4 Hz, 2H), 7.32 (t, J=7.0 Hz, 2H), 7.29-7.12 (m, 5H), 4.52-4.38 (m, 2H), 4.36-4.15 (m, 3H), 3.81-3.55 (m, 6H), 3.01 (dd, J=13.8, 4.9 Hz, 1H), 2.89-2.73 (m, 2H), 2.38 (dd, J=17.6, 5.5 Hz, 1H). ¹³C NMR (101 MHz, DMSO-d₆) δ 177.4, 176.4, 171.1, 169.6, 169.2, 168.5, 156.5, 143.9, 140.7, 137.6, 129.2, 128.2, 127.7, 127.1, 126.4, 125.3, 120.2, 65.8, 53.6, 49.5, 46.6, 43.5, 42.0, 41.8, 37.6, 35.9. IR (ATR-FTIR) 3301 (br), 3065 (w), 2950 (m), 2918 (m), 1719 (m), 1654 (s), 1528 (m), 1450 (m), 1375 (m), 1262 (m), 1201 (m), 741 (m). HRMS (ESI) (m/z) [M+Na] calculated for C₃₄H₃₄N₆O₈Na, 677.2330. found, 677.2322.

### Time-Course Study of Hydrolysis of C-Terminal Cyclic Imides

To a 1.7 mL microcentrifuge tube (Sorenson, catalog no. 11500) was added 1×PBS (998 μL), DMSO (1.00 μL), and 10 mM Fmoc-GGG-FcQ (SEQ ID NO: 8) or Fmoc-GGG-FcN (SEQ ID NO: 9) in DMSO (1.00 μL). The resulting solution were placed in a sand bath pre-heated at 37° C., and the reaction progress was monitored by Ultra performance liquid chromatography (UPLC) with 250-270 nm detection. Representative UPLC spectra for these experiments with peak assignments are given below. The conversion of the Fmoc-GGG-FcQ (SEQ ID NO: 8) or Fmoc-GGG-FcN (SEQ ID NO: 9) to its hydrolyzed form was calculated based on the integration of the corresponding peak relative to the integration of the corresponding starting material. Each reaction was performed in triplicate.

Note: Hydrolysis of Fmoc-GGG-FcQ (SEQ ID) NO: 8) or Fmoc-GGG-FcN (SEQ ID NO: 9) would be expected to yield a mixture of two corresponding constitutional isomers.¹¹⁶ In our study, these two hydrolysis products were not distinguished.

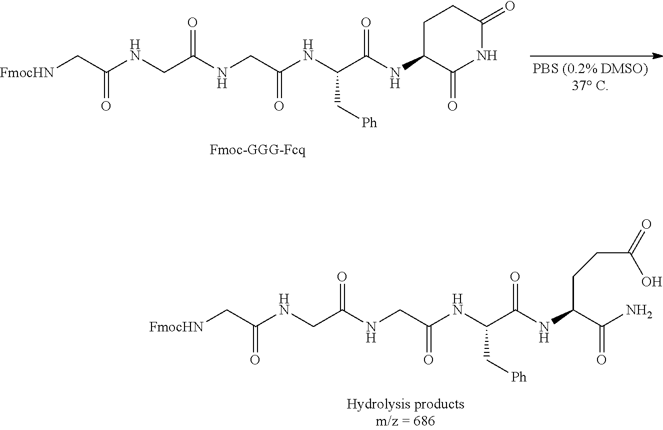

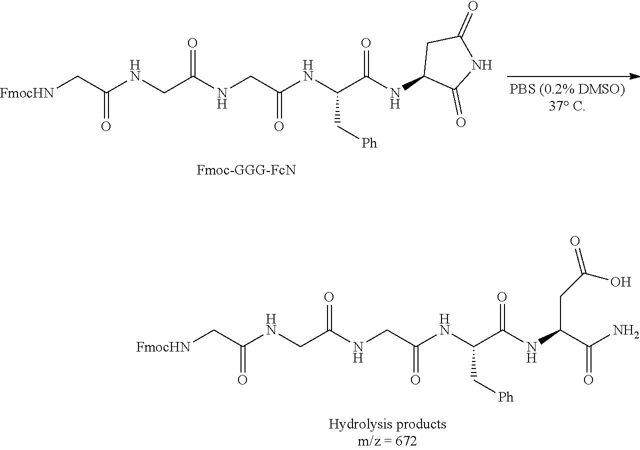

### Results

### C-Terminal Glutarimides are Functional CRBN Modulators in Targeted Protein Degradation

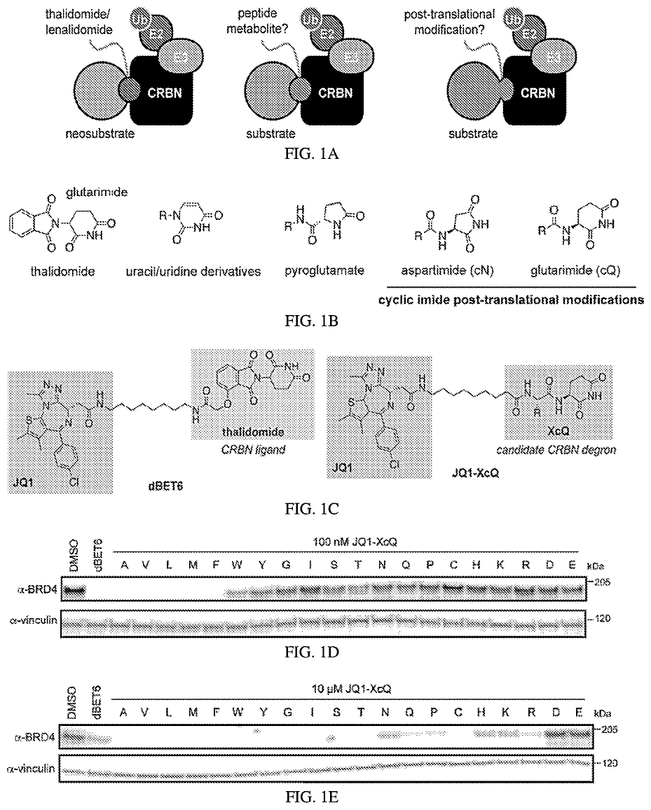

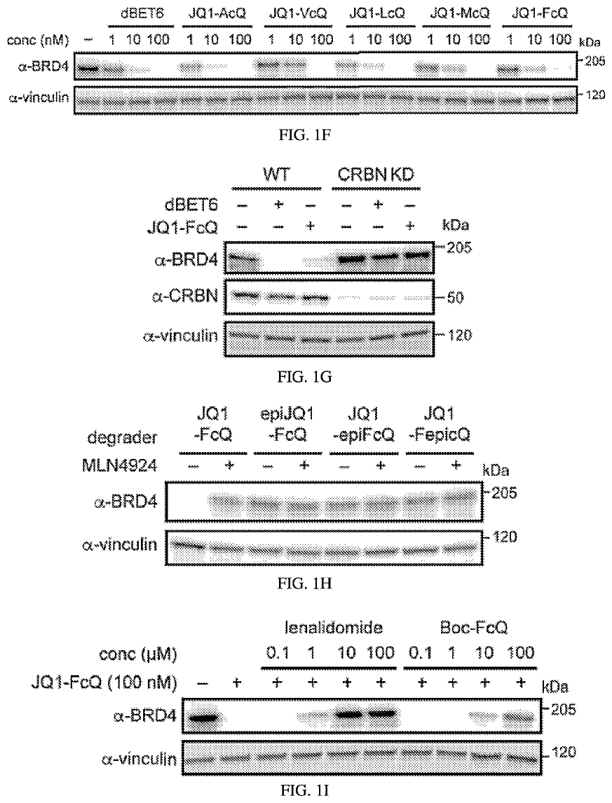

A physiologically relevant degron for the thalidomide binding domain of CRBN that promotes substrate degradation in cells was discovered. To circumvent this challenge, it was first set out to identify biomimetic ligands that functionally engage CRBN using a targeted protein degradation strategy. In this strategy, degradation of a known target protein by bifunctional small molecule degraders (e.g., PROTACs)²⁹ would report on functional engagement of CRBN in the CRL 4^{CRBN} complex in cells. Inspired by the flexible conversion of the bromodomain BRD4 inhibitor JQ1 into a BRD4 degrader by chemical functionalization with a thalidomide analog (e.g., dBET6),³⁰⁻³² analogous degraders were designed that replaced thalidomide with potential biomimetic ligands for CRBN (FIG. 1C). It was hypothesized that successful substitution of thalidomide with a biologically relevant structure would promote degradation of BRD4 and thus give a functional readout for CRBN engagement in cells.

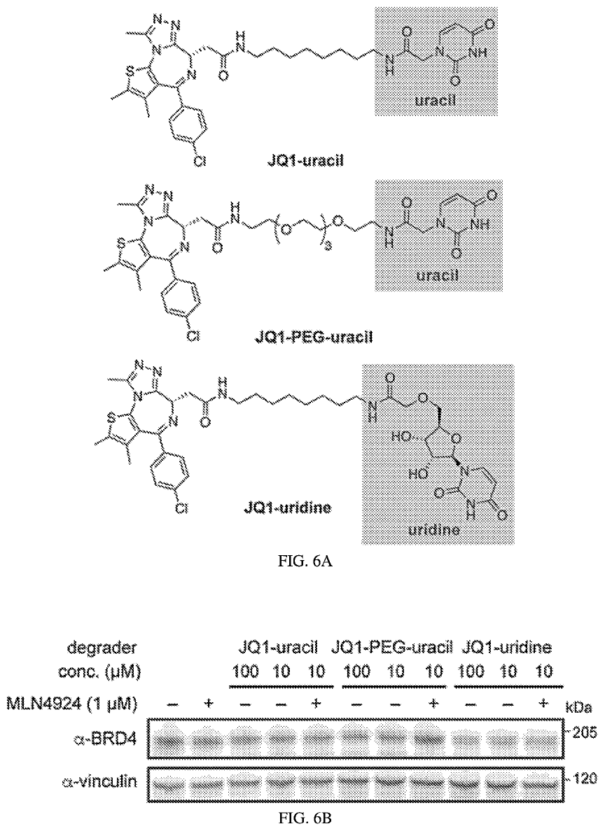

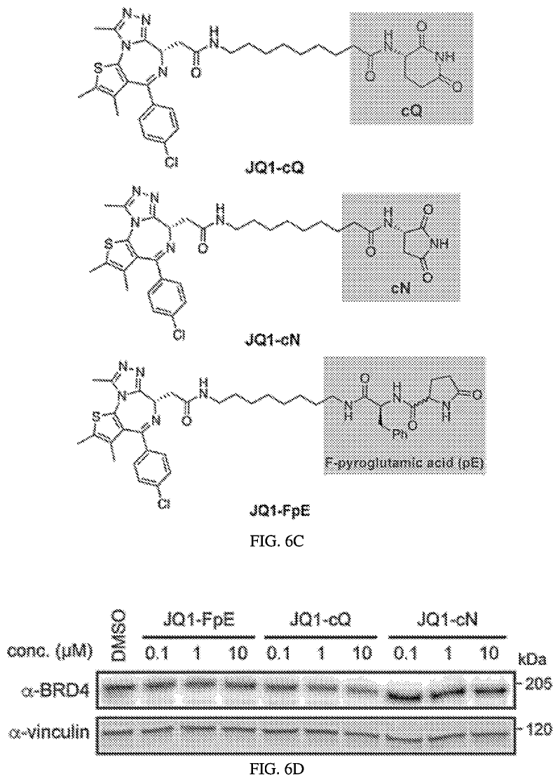

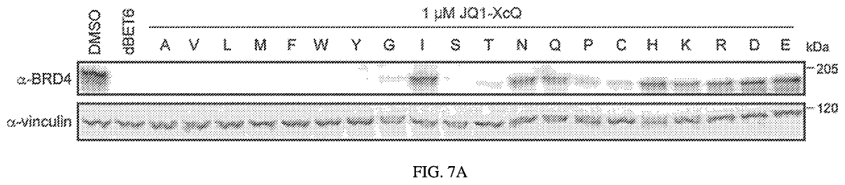

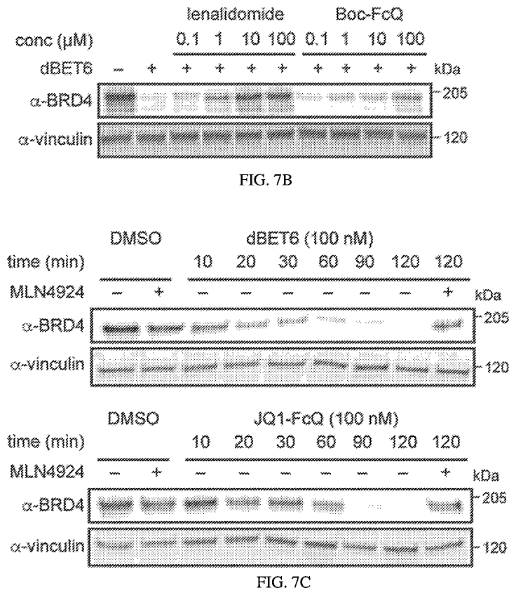

Initial evaluation of uridine-based probes revealed no functional engagement of CRBN leading to degradation of BRD4 in HEK293T cells (FIGS. 6A-6B). Ensuing evaluation of 15 candidate structures that resemble thalidomide eventually revealed a set of functional degraders consisting of JQ1 linked to a dipeptide with a C-terminal glutarimide (JQ1-XcQ, FIG. 1C). Substitution of the dipeptide degrader at the variable N−1 position (X) with each of the twenty amino acids showed that non-polar and aromatic amino acid side chains promoted high levels of degradation of BRD4 at 100 nM (FIG. 1D). The majority of dipeptide degraders were functional at 1 μM, and 18 out of 20 of the dipeptide degraders promoted degradation at 10 μM, with the exception of the negatively charged JQ1-DcQ and JQ1-EcQ (FIG. 1E, FIG. 7A). Interestingly, the hook effect resulting in reduced degradation of BRD4 can be observed from dBET6 at 10 μM, but not from the dipeptide degraders. The degradation efficiencies of the most potent dipeptide degraders JQ1-AcQ, JQ1-VcQ, JQ1-LcQ, JQ1-McQ, and JQ1-FcQ were further compared against dBET6 over a lower dose range of 1 to 100 nM (FIG. 1F). These dipeptide degraders exhibited dose-dependent degradation of BRD4 equivalent to or better than dBET6 in HEK293T cells. Degradation of BRD4 with JQ1-XcQ implicates the dipeptide motif for functional engagement of CRBN, as the cyclic imide alone did not promote degradation of BRD4 (FIGS. 6C-6D). Separately, an alternate glutamine-derived pyroglutamate dipeptide was also a non-functional degrader of BRD4 in cells (FIGS. 6C-6D). Based on these data and molecular similarity to thalidomide, the dipeptide FcQ was selected for further evaluation as a degron for CRBN.

To assess the dependence of JQ1-FcQ activity on CRBN engagement, wild-type HEK293T (WT) or HEK293T-CRBN knockdown (CRBN KD) cells³³ were treated with dBET6 or JQ1-FcQ. Degradation of BRD4 by both compounds was lost upon shRNA knockdown of CRBN (FIG. 1G). Epimerization of JQ1-FcQ at each of the three stereocenters (in JQ1, Phe, or cQ) inactivated the degrader, indicating that degradation occurs via the natural stereochemistry of the amino acids for optimal recognition by CRBN and engagement of BRD4 (FIG. 1H). Finally, addition of lenalidomide or Boc-protected FcQ (Boc-FcQ) competitively inhibited BRD4 degradation by JQ1-FcQ in a dose-dependent manner, indicating that the thalidomide binding site of CRBN is engaged by glutarimide ligands (FIG. 1I). Similarly, lenalidomide and Boc-FcQ competitively inhibited degradation of BRD4 in the presence of dBET6 (FIG. 7B). Degradation of BRD4 by dBET6 and JQ1-FcQ displayed similar kinetics over time, resulting in complete degradation within 90 min in a Cullin-dependent manner (FIG. 7C). Collectively, these data establish FcQ and glutarimide-based dipeptides more broadly as successful substitutes for thalidomide that functionally engage CRBN in cells.

### C-Terminal Aspartimides are Substitutes for Thalidomide in Targeted Protein Degradation

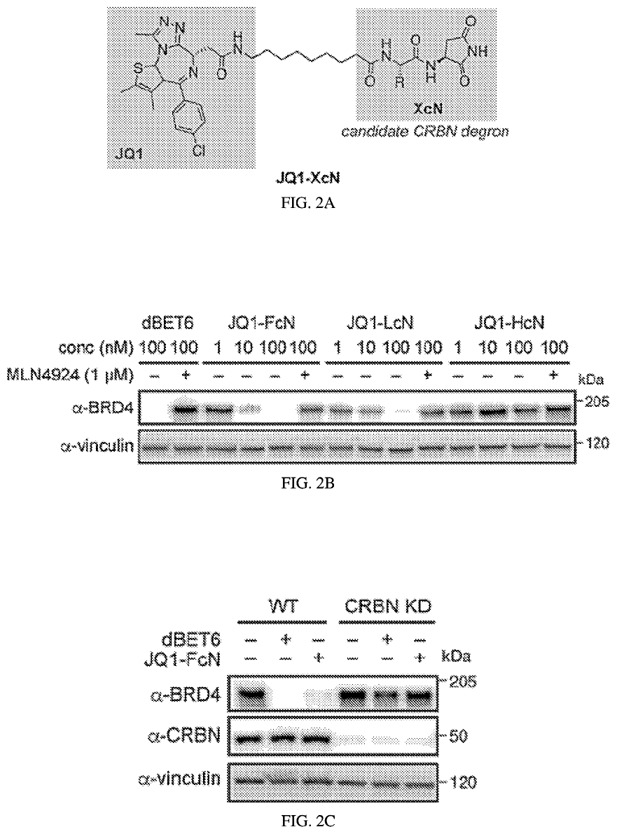

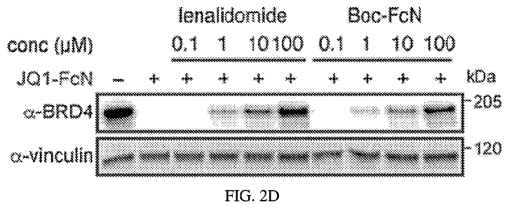

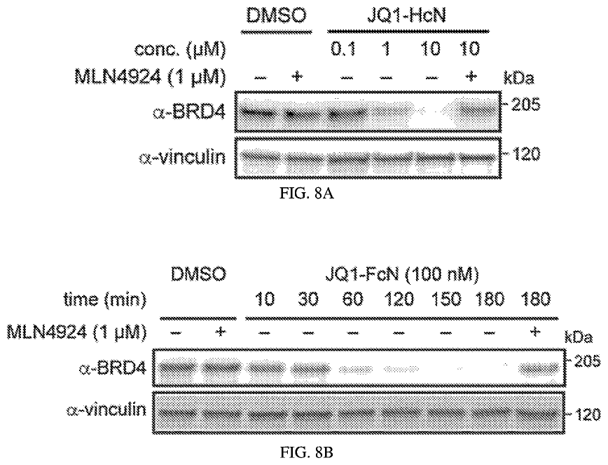

Next, it was evaluated whether the aspartimide, a second class of cyclic imides derived from asparagine, was also a functional substitute of thalidomide due to their chemical similarity and the reported formation of C-terminal aspartimides in long-lived proteins and protein splicing products (e.g., inteins), which are typically promoted by a penultimate histidine. Therefore, aspartimide-based dipeptide degraders were constructed for evaluation in BRD4 degradation assays in cells (FIG. 2A). Using insights from the glutarimide-based dipeptide degraders, JQ1-XcN degraders embedded with Phe, Leu, and His at the N−1 position (X) were selected. It was observed that degradation of BRD4 by the aspartimide-based dipeptide degraders was comparable to the glutarimide-based dipeptide degraders (FIG. 2B). As before, substitution of thalidomide with FcN and LcN yielded a degrader that was equivalent in efficiency to dBET6 over 1-100 nM concentrations, while substitution of thalidomide with HcN, yielded degradation of BRD4 over 1-10 μM concentrations (FIG. 2B, FIG. 8A). As the efficiency of the selected JQ1-XcN degraders is analogous to JQ1-XcQ degraders in the degradation of BRD4, engagement of CRBN by C-terminal aspartimides and glutarimides is comparable.

Closer investigation of JQ1-FcN showed that degradation of BRD4 by JQ1-FcN was completed within 2 h in HEK293T cells and was blocked by knockdown of CRBN (FIG. 2C, FIG. 8B). Likewise, lenalidomide and the dipeptide Boc-FcN were competitive inhibitors of BRD4 degradation, presumably via disruption of JQ1-FcN engagement of CRBN (FIG. 2D). These data indicate that aspartimide-based dipeptides are analogous to glutarimide-based dipeptides in terms of their cellular interaction with CRBN and are also functional substitutes for thalidomide in targeted protein degradation. Efforts were then focused on further characterization of both 5- and 6-membered C-terminal cyclic imides as degrons recognized by CRBN.

### Ternary Complex Formation Mediated by Cyclic Imide Dipeptide Degraders

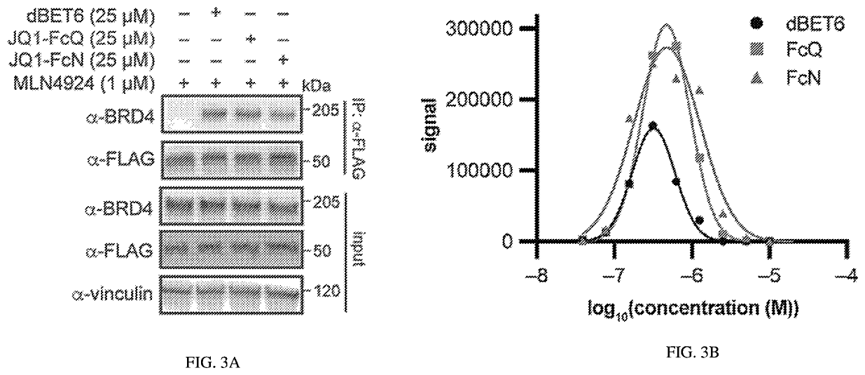

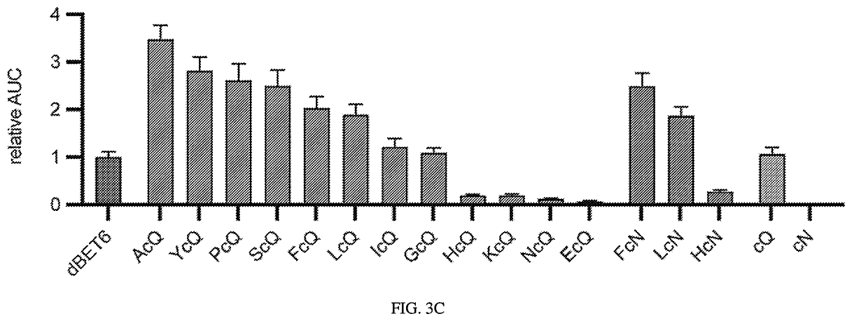

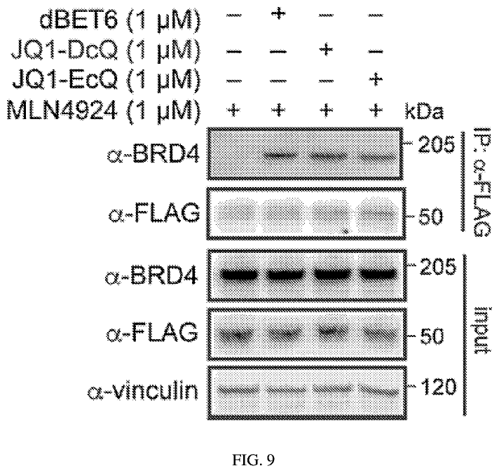

The cyclic imide degrons were characterized in comparison to thalidomide by investigating the propensity of the different degraders for direct engagement of CRBN and ternary complex formation with BRD4, as cellular degradation of BRD4 by chemical degraders is a composite of cellular accessibility, ternary complex engagement, and orientation of the complex for ubiquitylation,^{32,34} each of which may influence the effective degradation efficiency. The target protein BRD4 was readily co-immunoprecipitated by FLAG-CRBN from a stable HEK293T cell line (HEK-CRBN)³³ upon treatment with 25 μM dBET6, JQ1-FcQ, or JQ1-FcN, indicative of ternary complex formation driving BRD4 degradation in cells (FIG. 3A). Ternary complex formation of the weakest cellular dipeptide degraders JQ1-DcQ and JQ1-EcQ were investigated in vitro to circumvent any interference from cellular access. HEK-CRBN lysates were treated with dBET6, JQ1-DcQ, or JQ1-EcQ at 1 μM for 2 h and the three compounds promoted equivalent co-immunoprecipitation of BRD4 with FLAG-CRBN (FIG. 9). The ability of these dipeptide degraders, which exhibit a range of cellular degradation efficiencies for BRD4, to form a ternary complex implies that CRBN has a broad and flexible ligand scope for C-terminal cyclic imide ligands.

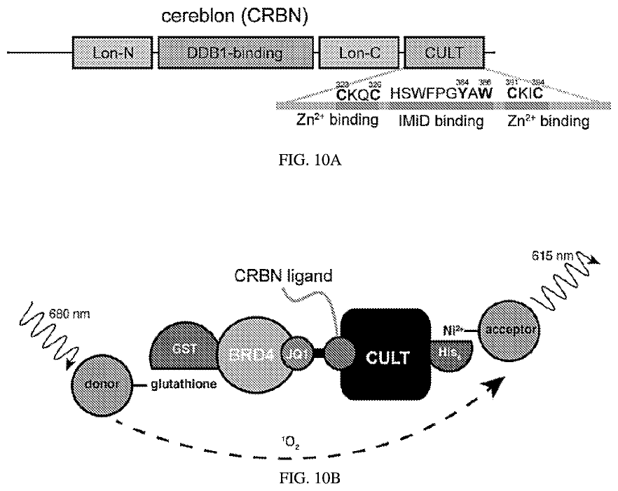

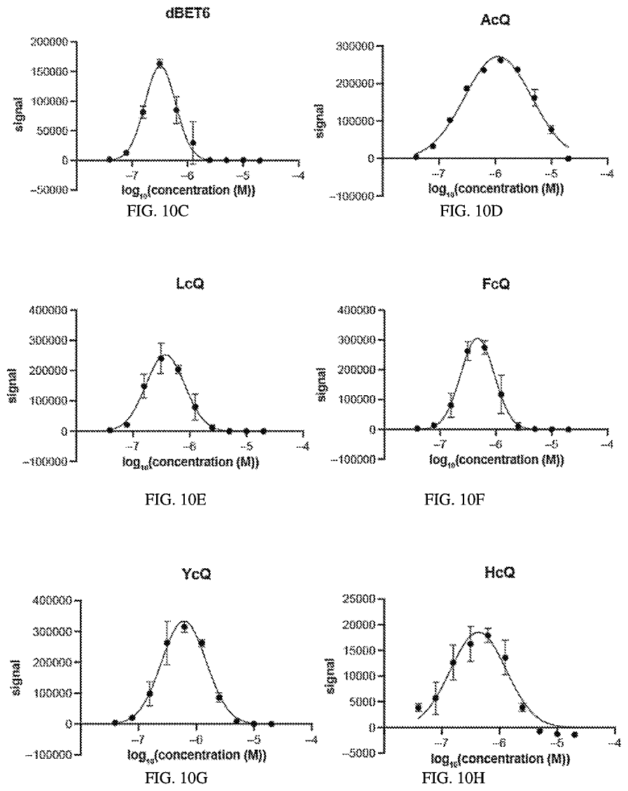

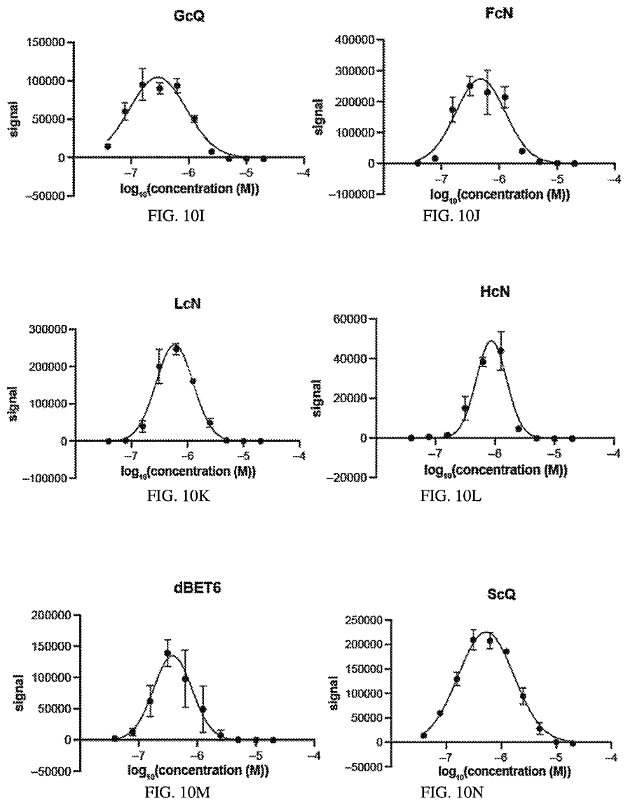

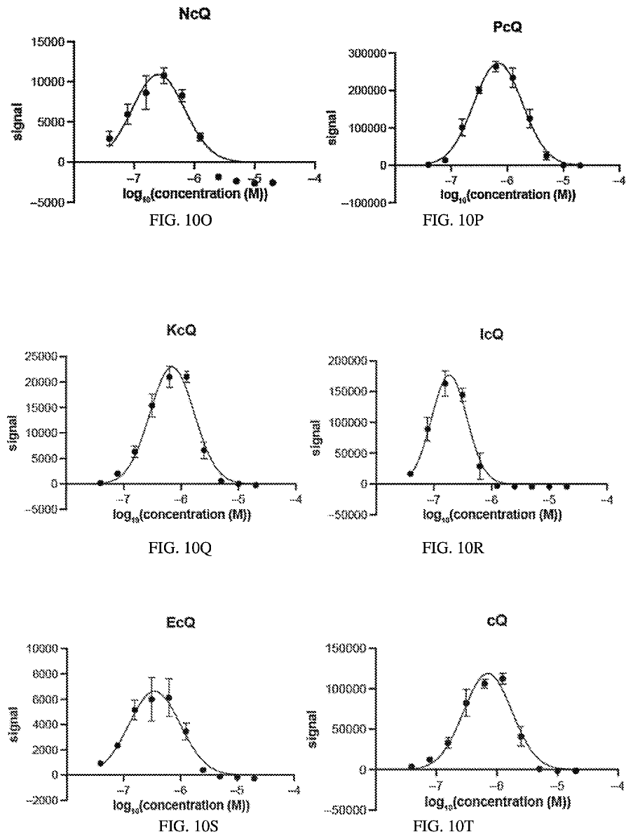

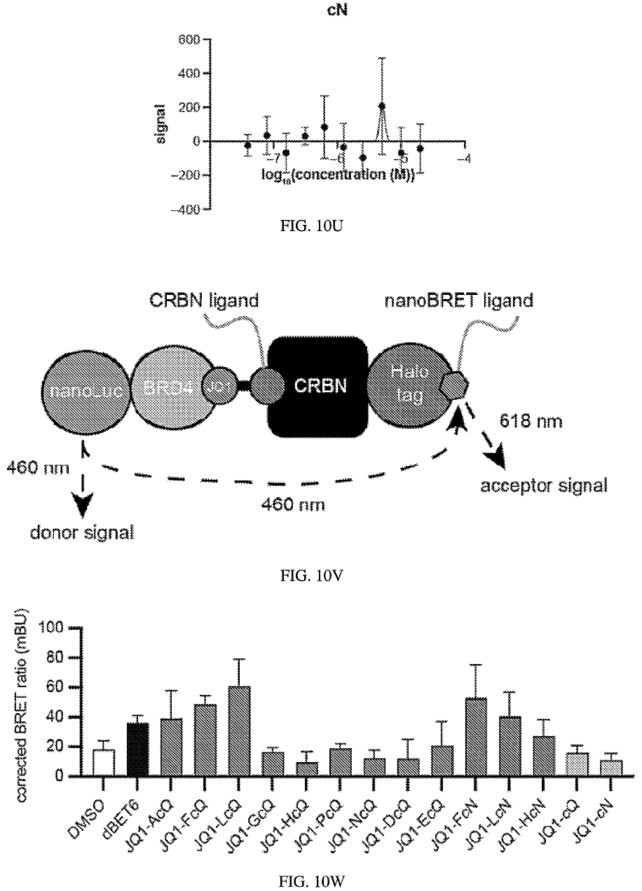

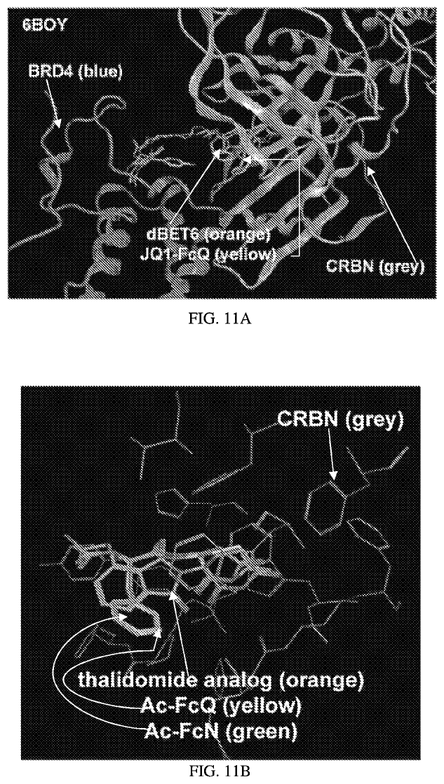

To further evaluate engagement of CRBN by the dipeptides, ternary complex formation mediated by the dipeptide degraders was systemically measured with recombinant GST-BRD4 and His-CRBN/DDB1, using AlphaScreen, which produces a signal if the degrader recruits GST-BRD4 and His-CRBN/DDB1 in close proximity (FIG. 10A-10B). The dipeptide degraders tested induced ternary complex formation, with several of the dipeptide degraders resulting in signal greater than or comparable to dBET6 (FIGS. 3B-3C, FIGS. 10C-10L). Direct comparison of dBET6 with JQ1-FcQ and JQ1-FcN showed that both dipeptide degraders induced a stronger ternary complex than dBET6 (FIG. 3B). Comparison of the relative area under the curve elicited by dipeptide degraders reflected the cellular trends in that degraders with non-polar or aromatic amino acid side chains at the N−1 position tended to promote ternary complex formation in vitro (FIG. 3C). Aspartimide-based degraders induced comparable ternary complex formation to the analogous glutarimide-based degraders, in alignment with BRD4 degradation observed in cells. Interestingly, it was found that the inactive degrader JQ1-cQ promotes ternary complex formation equivalent to the active JQ1-GcQ, potentially indicating that the additional amino acid positions the rest of the ternary complex in a more productive conformation for ubiquitylation and degradation in cells. Measurement of ternary complex formation in cells using a NanoBRET assay revealed similar trends (FIGS. 10V and 10W). In sum, these data illustrate that all of the dipeptide degraders directly engage CRBN and that the efficiency of ternary complex formation broadly corresponds with the observed cellular activity.

### Selectivity and Transferability of the Cyclic Imide Degrons for Substrate Degradation

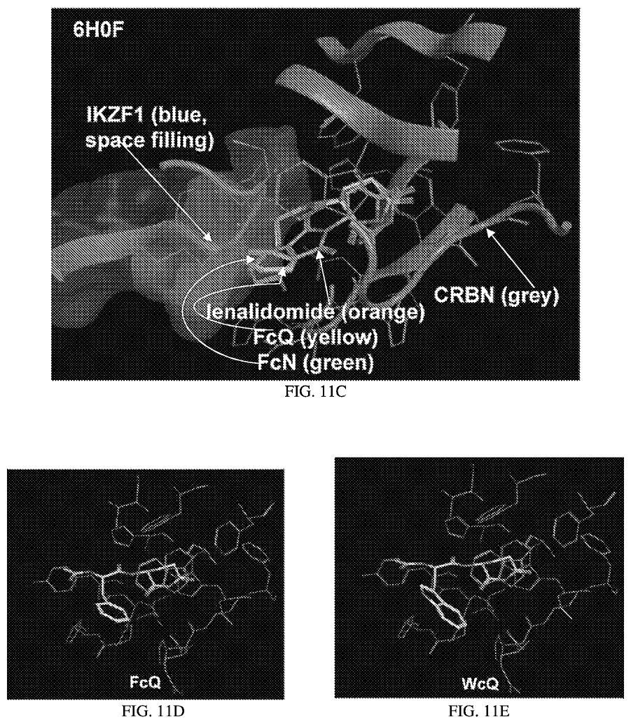

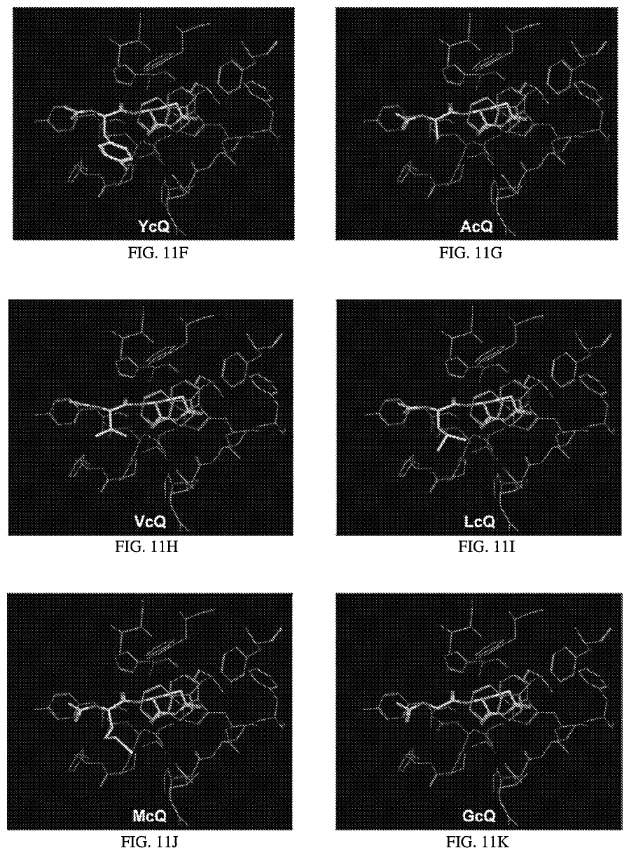

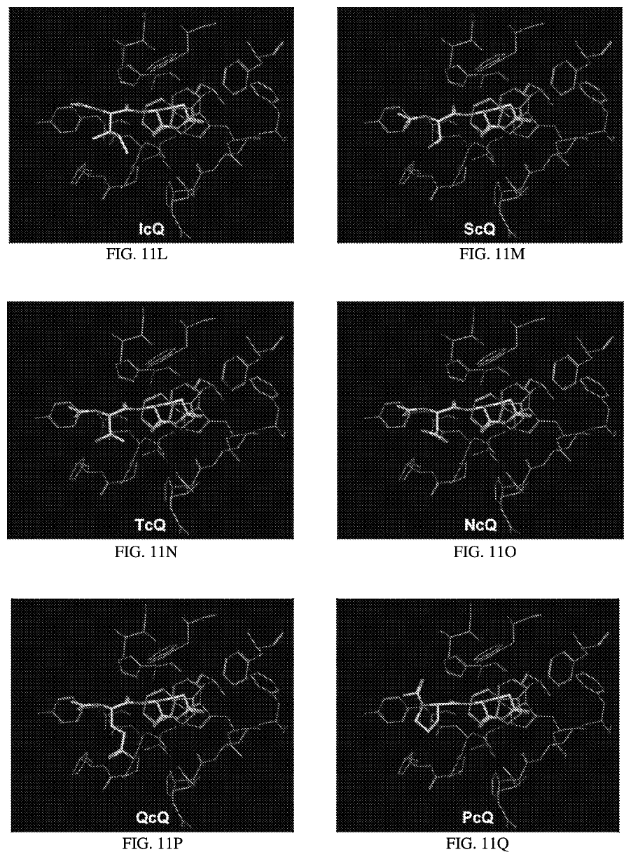

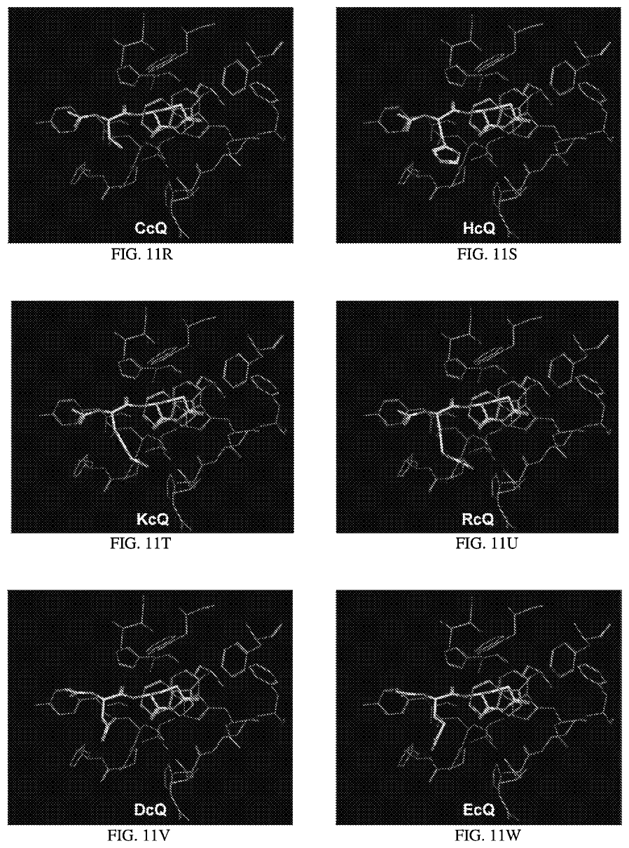

It was then investigated whether the cyclic imide-containing dipeptides act as molecular glues that induce substrate recruitment for ubiquitylation by the CRL4^{CRBN} E3 ligase complex, as depicted in one of the two models for how thalidomide could mimic the native CRBN degron (FIG. 1A). This model is analogous to the mechanism of thalidomide and its derivatives, which directly mediates substrate degradation through a beta-hairpin motif in a CRBN-dependent manner.³⁶ To predict whether the dipeptides would recruit similar substrates as thalidomide, a molecular modeling of dBET6 with JQ1-FcQ and JQ1-FcN was performed in the ternary complex with CRBN and BRD4 and extended this model to the glutarimide dipeptides (FIGS. 11A-11W). These models indicated that the imide region of the dipeptide binds to CRBN in a similar manner as thalidomide, but the amino acid at the N−1 position occupies a distinct molecular space relative to the phthalimide region of thalidomide (FIGS. 11A-11C). The dipeptides are therefore unlikely to stabilize the same beta-hairpin motifs as the IMiDs, but may mediate degradation of unique substrates.

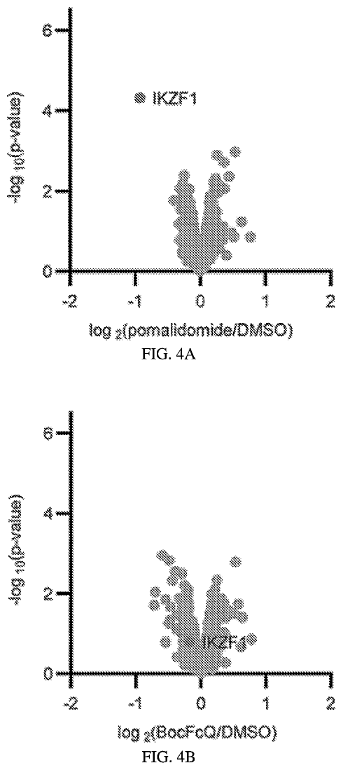

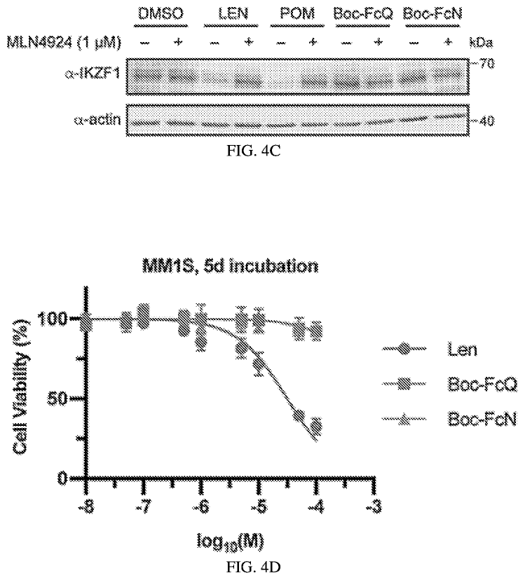

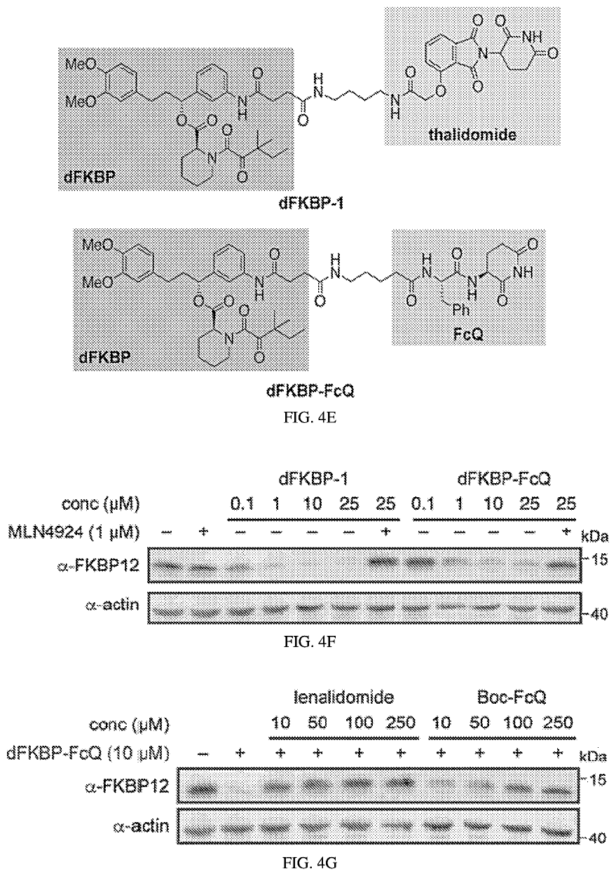

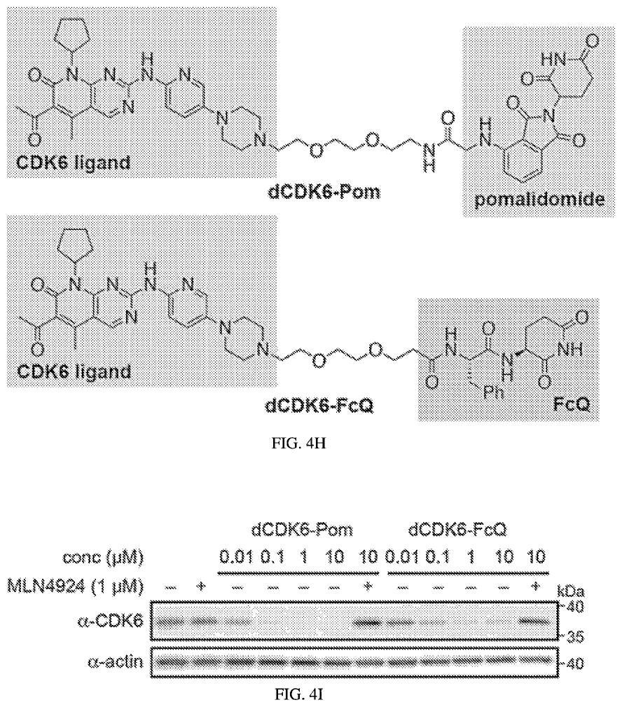

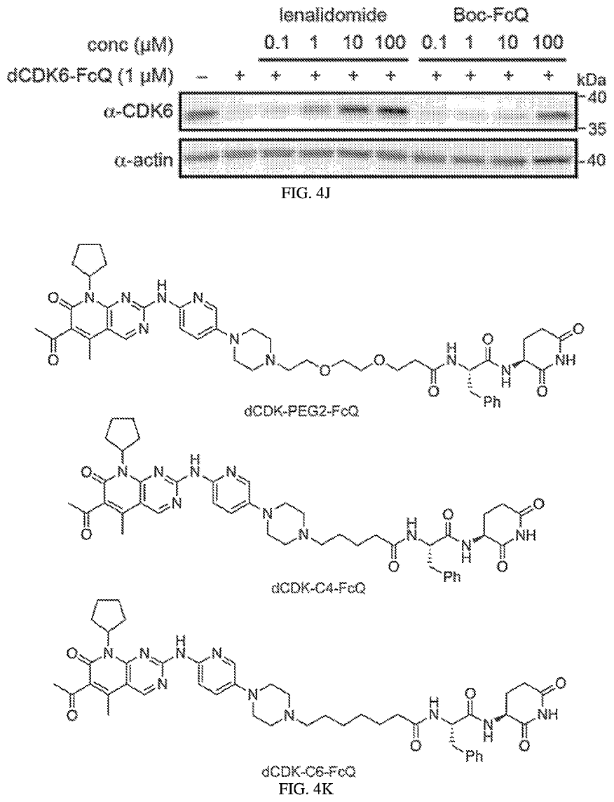

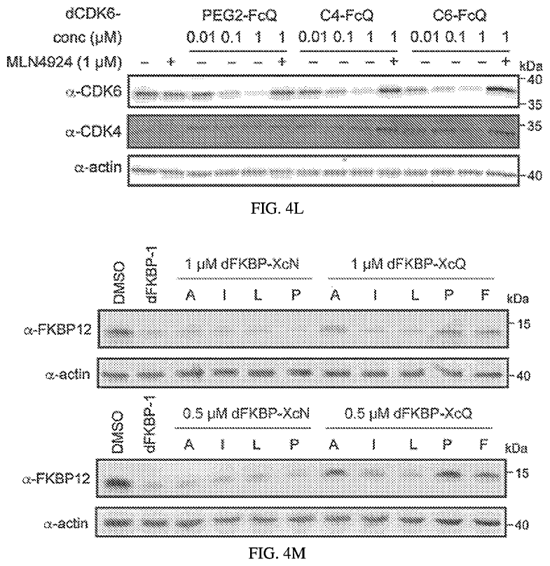

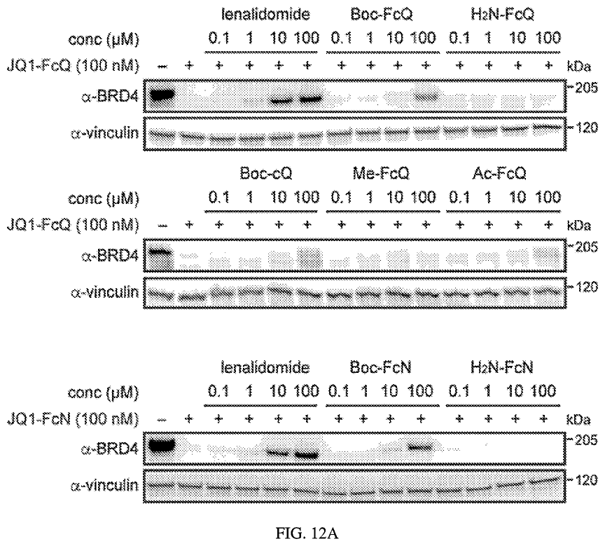

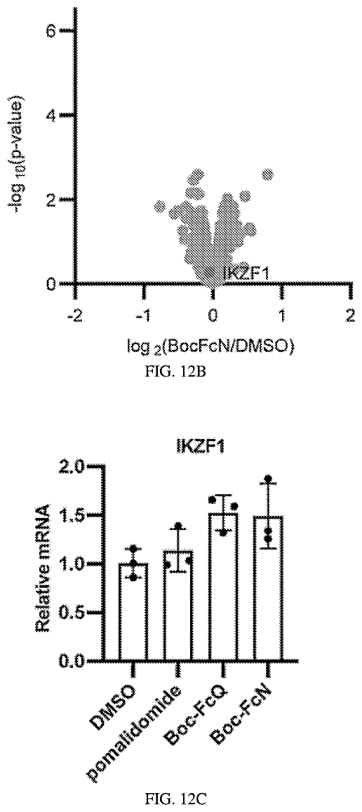

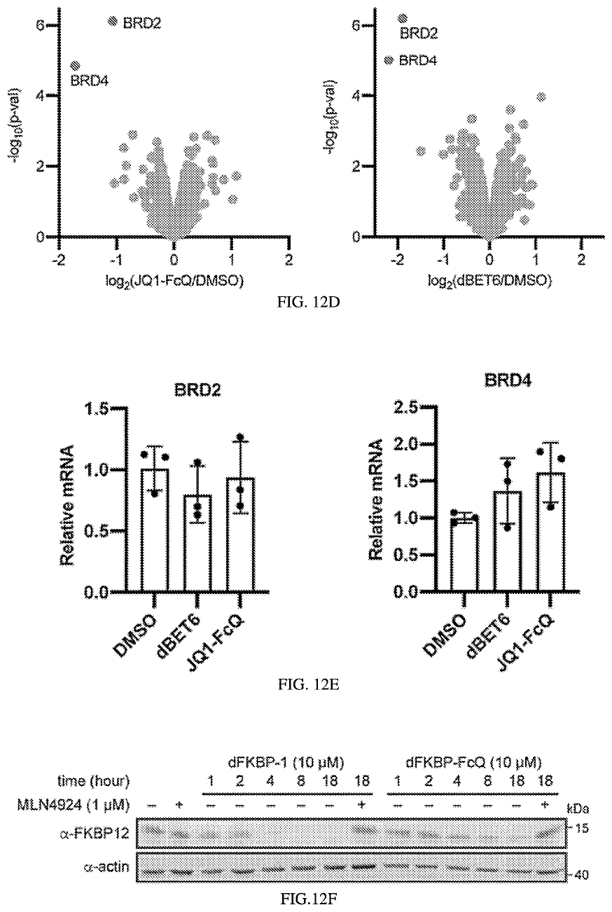

To evaluate whether the dipeptides independently promote substrate degradation using global proteomics, the competitive inhibition of BRD4 degradation by dBET6 in cells was examined, and it was found that the Boc-protected dipeptide (Boc-FcQ) was an effective competitor in cells (FIG. 12A). The multiple myeloma cell line MM.1S was treated with 10 μM of pomalidomide or a dipeptide (Boc-FcQ or Boc-FcN) for 10 h and evaluated by global proteomics to identify candidate substrates degraded by the dipeptides. Treatment with pomalidomide showed selective degradation of the substrate IKZF1 (FIG. 4A).^{2,3} However, IKZF1 was not susceptible to degradation in the presence of Boc-FcQ or Boc-FcN and no additional substrates were degraded upon treatment with the dipeptides (FIG. 4B, FIG. 12B). Indeed, IKZF1 was degraded upon treatment of MM.1S cells with lenalidomide and pomalidomide, but not with the dipeptides (FIG. 4C). Protein expression levels were similar across treatments (FIG. 12C). The differences in substrate degradation extend to differences in anti-proliferative effects, as MM.1S cells were sensitive to lenalidomide treatment (IC₅₀=31.6 μM), but not to Boc-FcQ or Boc-FcN (IC₅₀>100 μM, FIG. 4D). The absence of substrate degradation observed with Boc-FcQ translated to high target protein selectivity from the degrader JQ1-FcQ. After treatment with thalidomide-based dBET6 or dipeptide-based JQ1-FcQ, the proteome of HEK293T cells was evaluated by global quantitative proteomics (FIG. 12D). Both degrader molecules were highly selective and effective in depleting BRD2 and BRD4 as shown by global proteomics, while protein expression levels of BRD2 and BRD4 were similar across treatments (FIG. 12E). Taken together, these data indicate that the dipeptide FcQ does not degrade additional substrates either independently or in the context of targeted protein degradation in MM.1S or HEK293T cells, suggesting that binding of the dipeptide to CRBN does not induce a novel degron.

Engineered Proteins with C-Terminal Imides are Substrates for Degradation by Cereblon

With evidence that cyclic imide dipeptides are functional ligands of CRBN at the thalidomide binding site and are readily embedded to a small molecule degrader to induce degradation of multiple targets, it was next sought to characterize whether the dipeptides FcQ and FcN can be incorporated into a protein substrate and thus act as a degron for the protein.

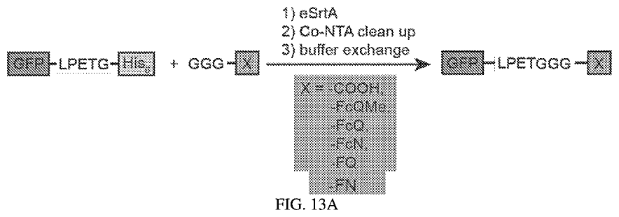

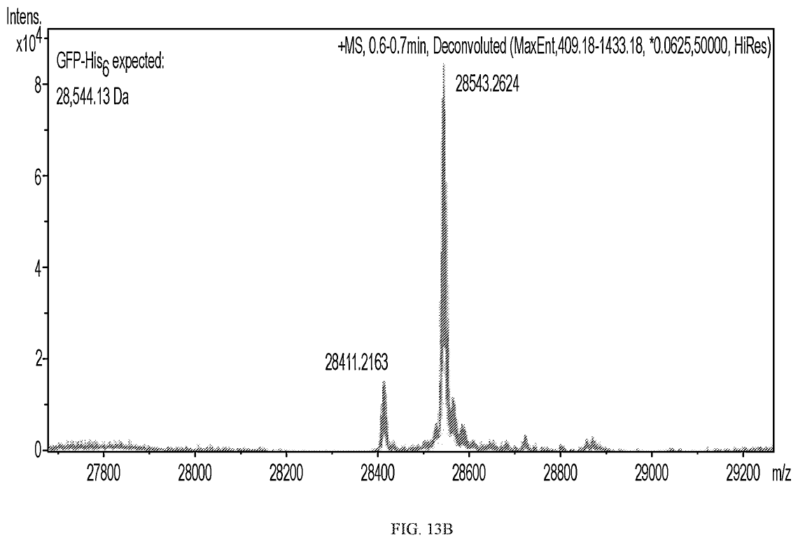

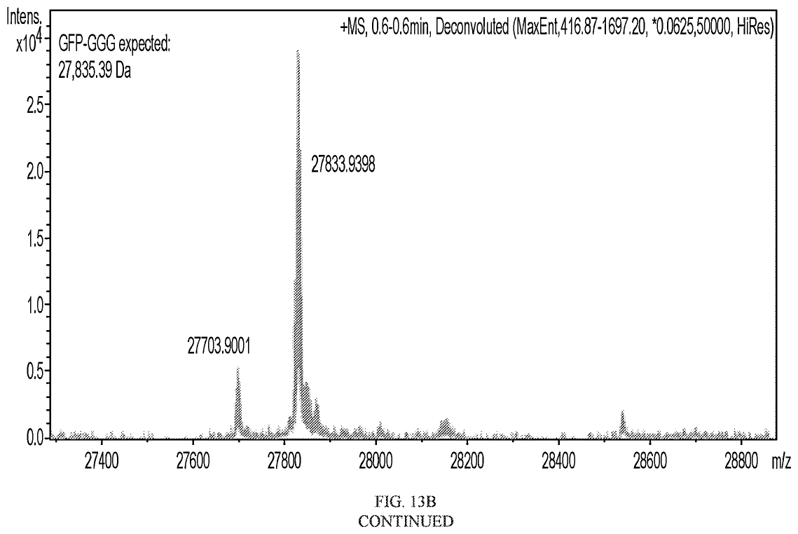

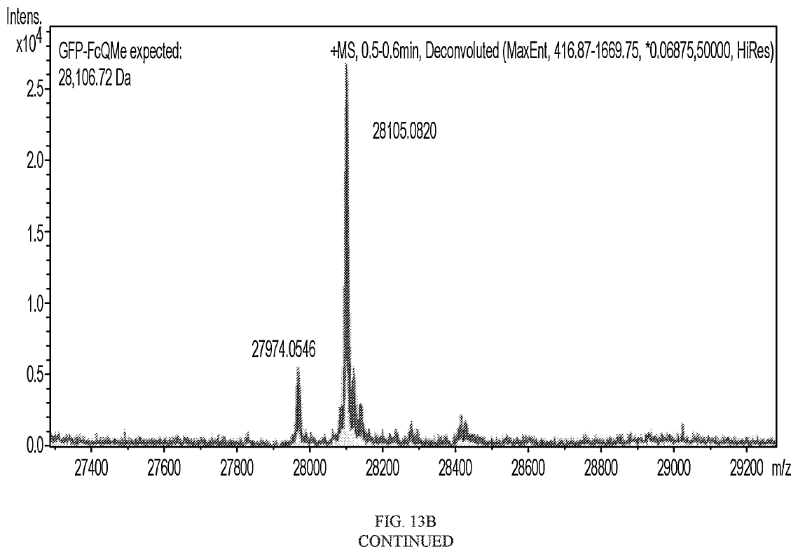

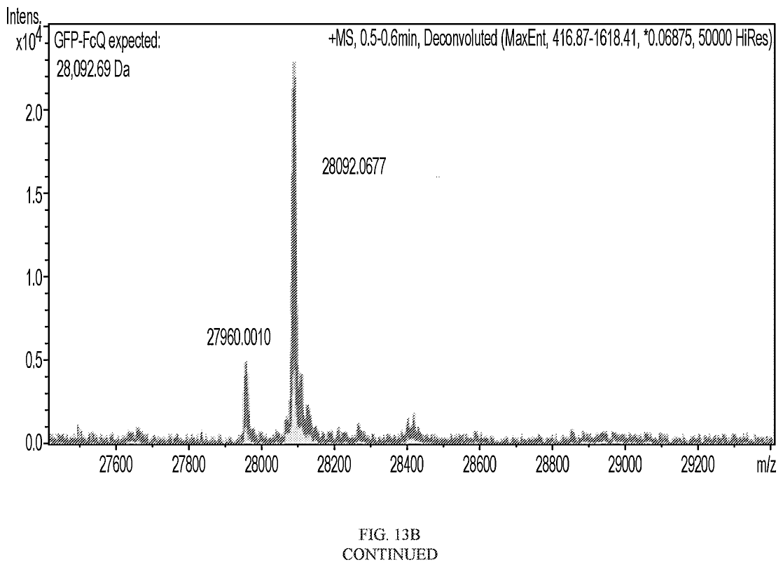

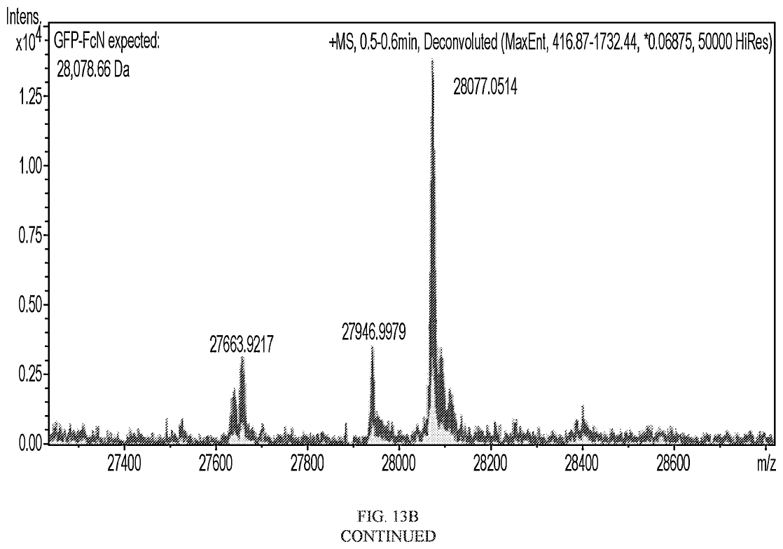

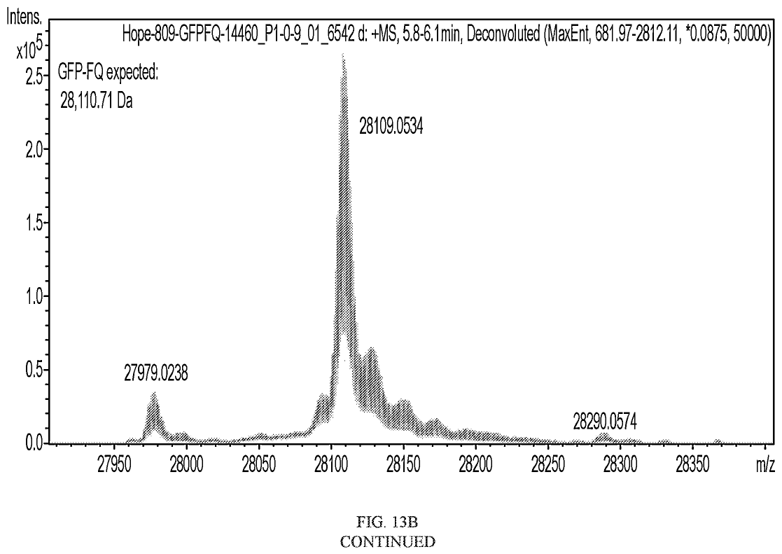

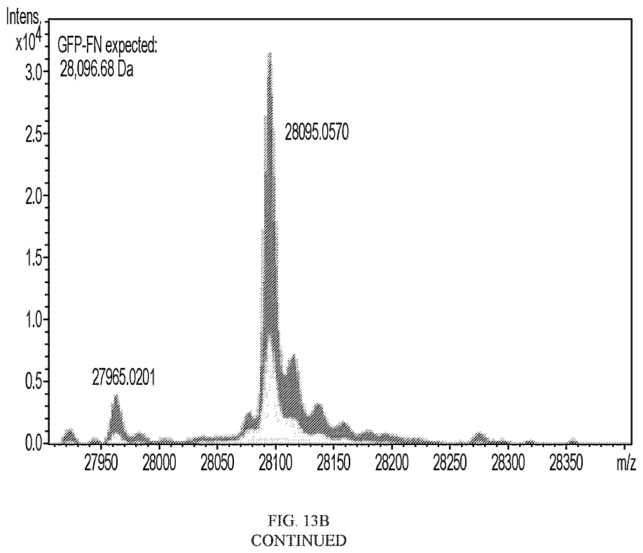

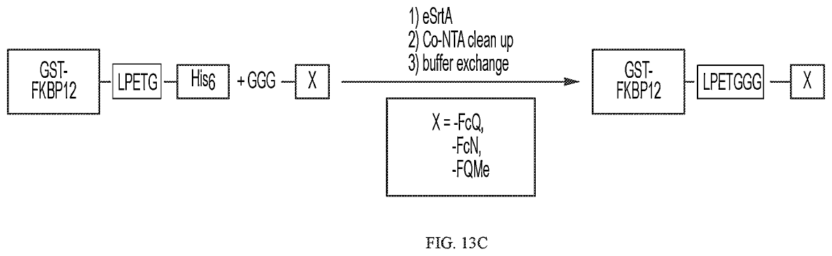

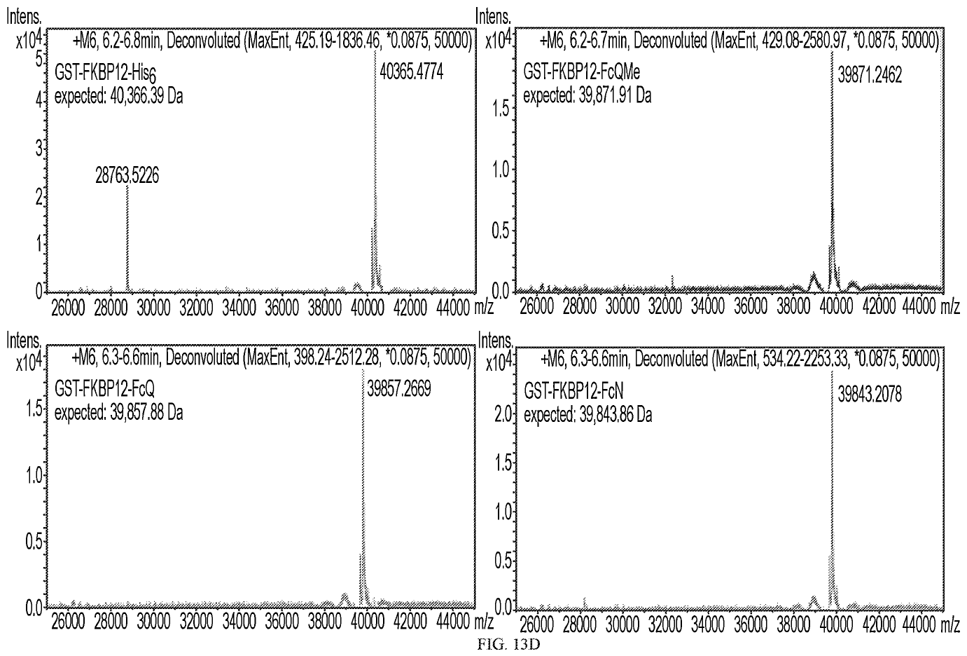

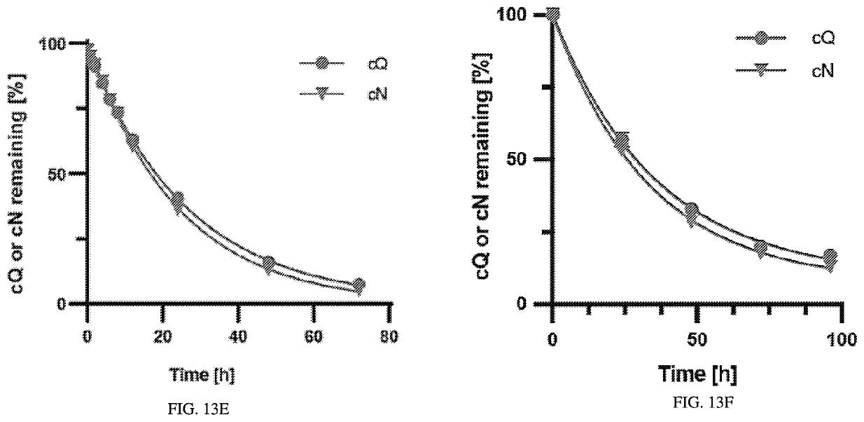

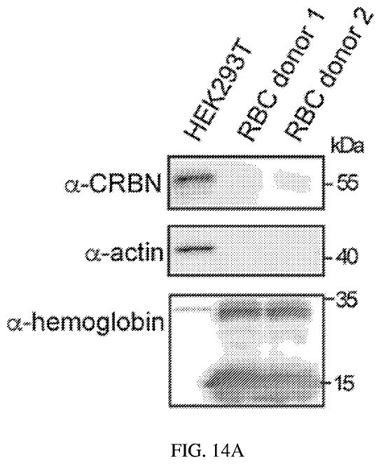

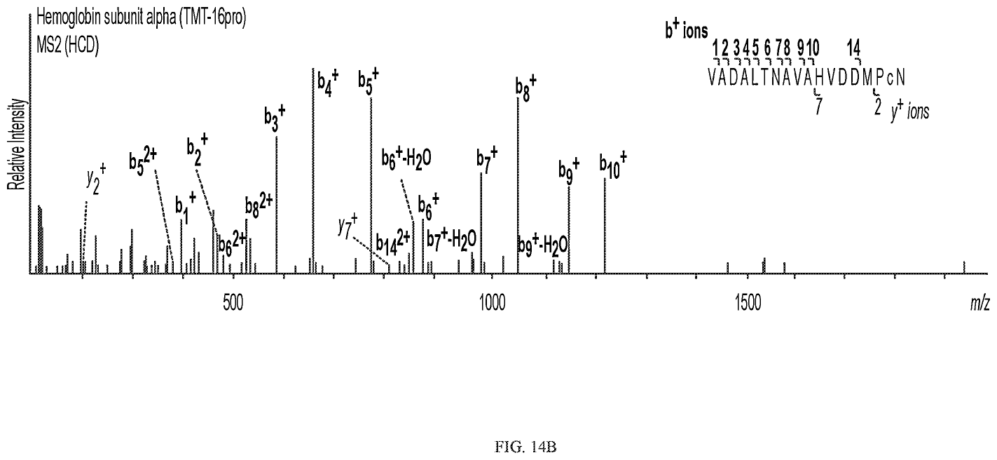

To generate the protein substrate, GFP was selected as an initial target. Using the sortase system,⁵¹ the C-terminal cyclic imide was installed on GFP to afford semi-synthetic GFP-FcQ or GFP-FcN. An inactive methylated glutarimide, GFP-Me, was also constructed with GFP-GGG as negative control (FIGS. 13A-13B).

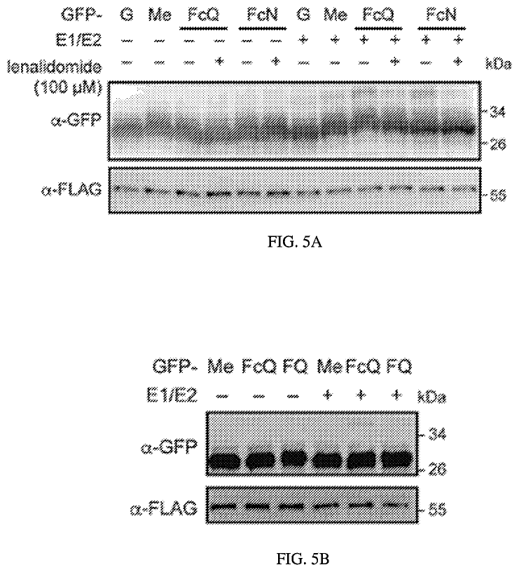

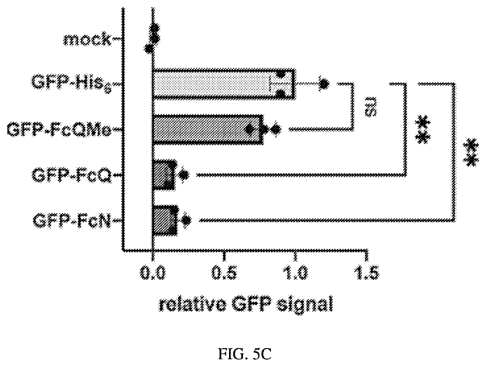

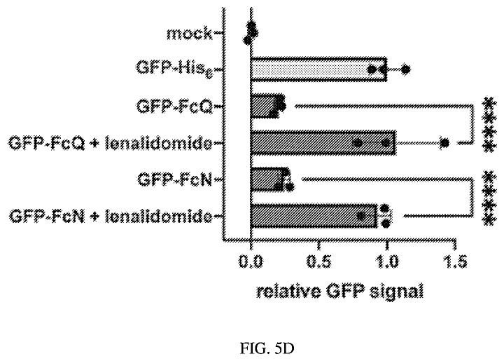

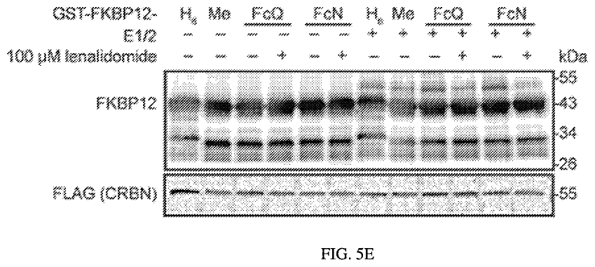

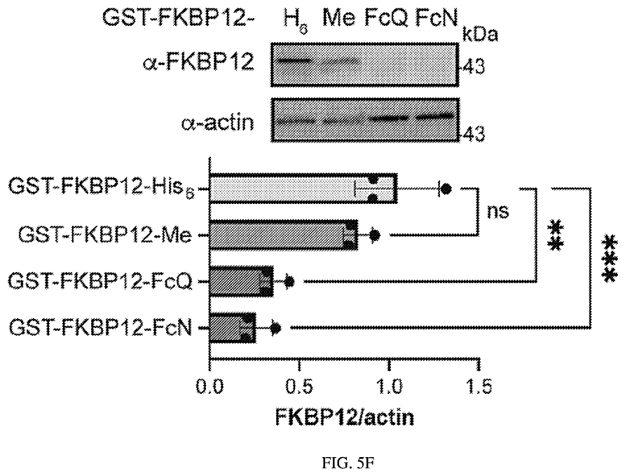

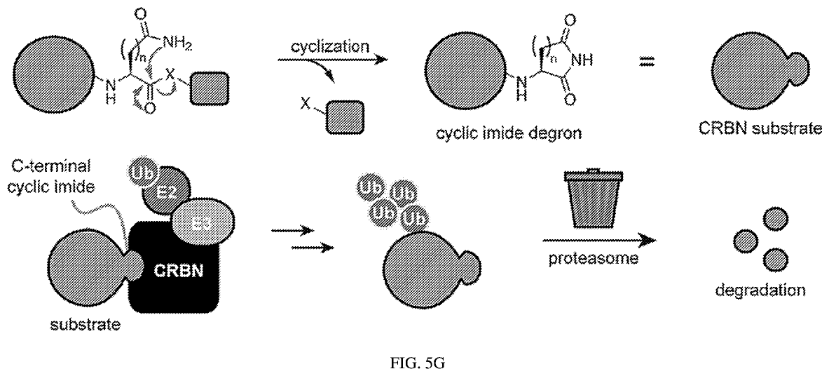

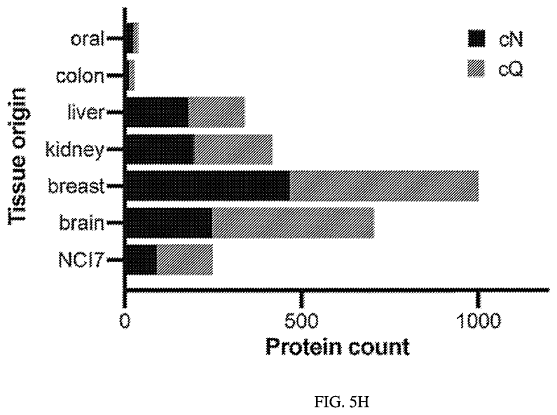

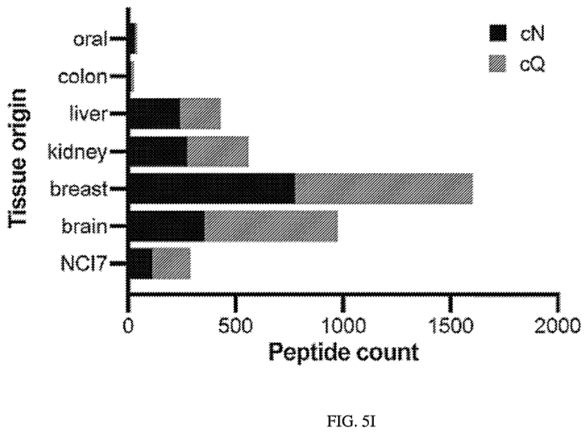

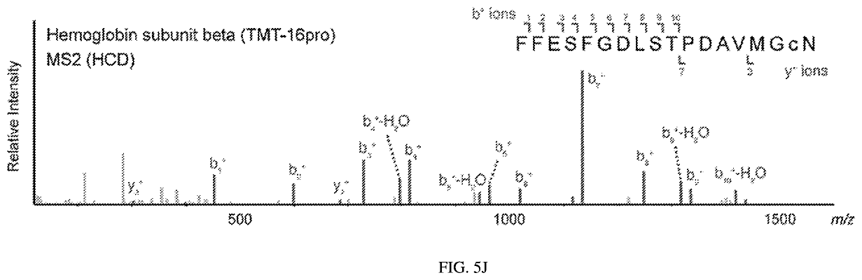

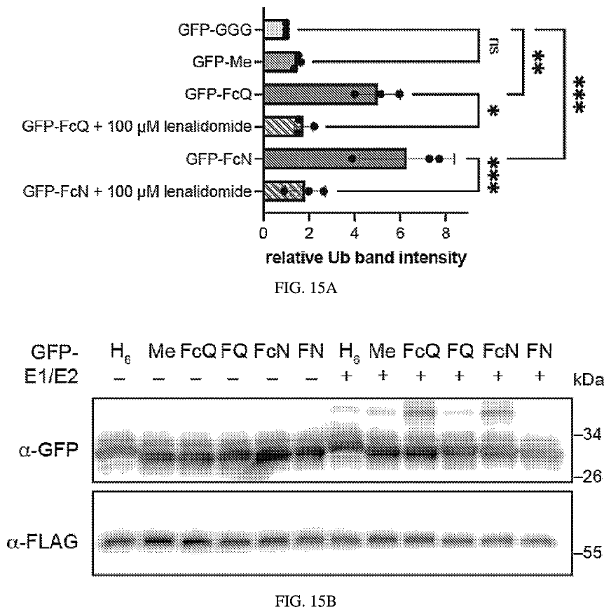

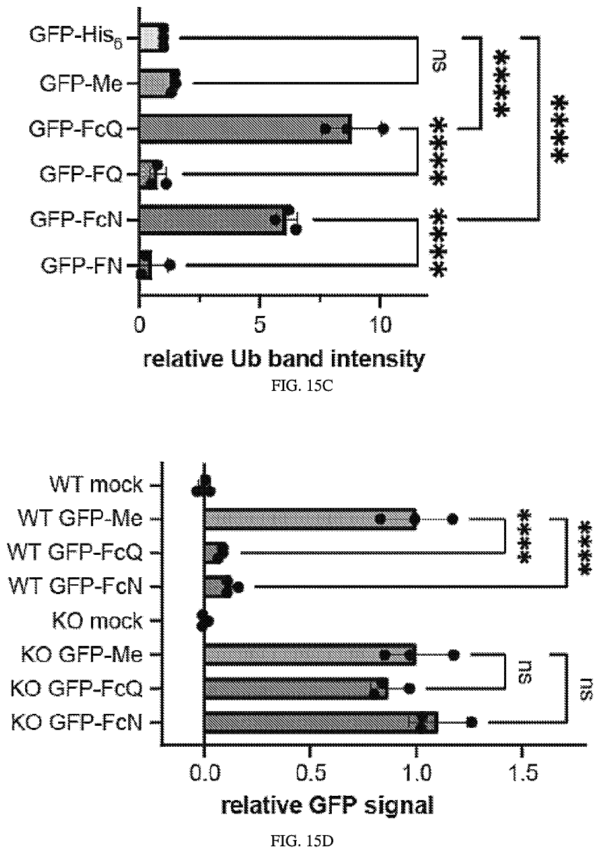

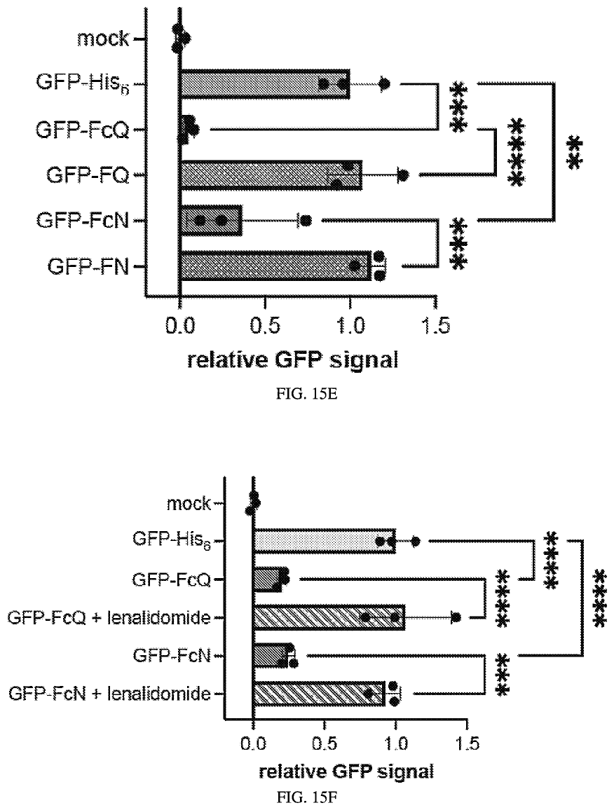

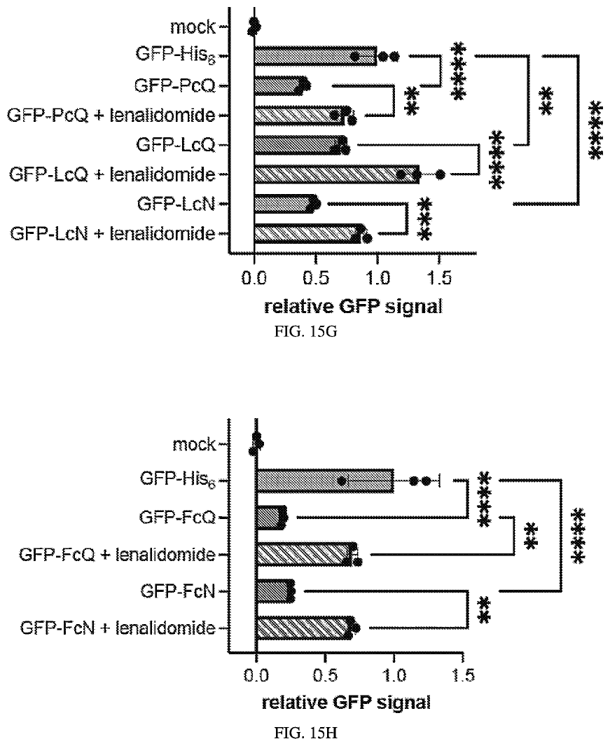

The semi-synthetic C-terminally modified GFPs were initially evaluated as potential substrates for the CRL4^{CRBN} complex by examining their susceptibility to ubiquitylation in vitro. The CRL4^{CRBN} complex isolated from HEK-CRBN cells selectively transferred ubiquitin to GFP-FcQ and GFP-FcN, but not to GFP-Me or GFP-GGG (FIG. 5A and FIG. 15A). In vitro ubiquitylation of GFP-FcQ and GFP-FcN was competitively inhibited by co-treatment with lenalidomide. To ensure the relevance of the cyclic imide, GFPs tagged with uncyclized glutamine and asparagine, GFP-FQ and GFP-FN, were evaluated as substrates for in vitro ubiquitylation and observed no modification by the CRL4^{CRBN} complex (FIG. 5B, FIG. 15B, and FIG. 15C).

Next, it was assessed whether the cyclic imide is a degron for CRBN in cells by introduction of the modified GFPs into HEK293T cells by electroporation. Analysis of cellular GFP levels after 6 h revealed lower GFP levels when GFP-FcQ or GFP-FcN carrying the CRBN degrons was delivered to cells relative to the unmodified GFP (GFP-His₆) or GFP-FcQMe (FIG. 5C), and the degradation was commuted with the genetic knockout of CRBN (FIG. 15D). Introduction of GFP-FQ and GFP-FN to HEK293T cells showed no difference in overall protein levels relative to GFP-His6, indicating that the rapid degradation of GFP-FcQ and GFP-FcN is dependent on the C-terminal cyclic imide (FIG. 15E). Inhibition of CRBN by cotreatment with lenalidomide competitively inhibited depletion of GFP-FcQ and GFP-FcN (FIG. 5D and FIG. 15F).

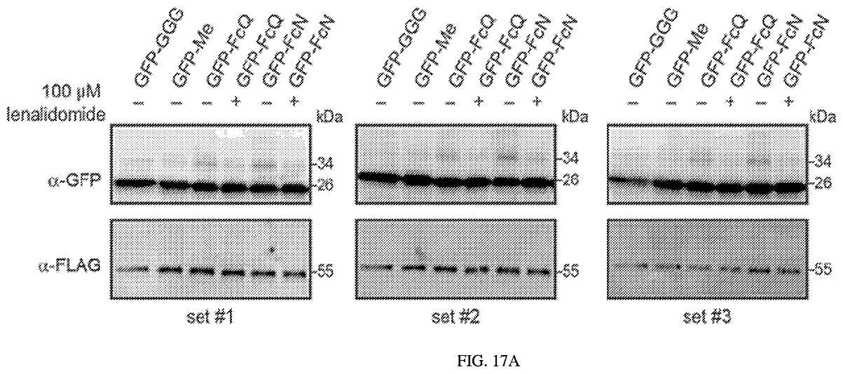

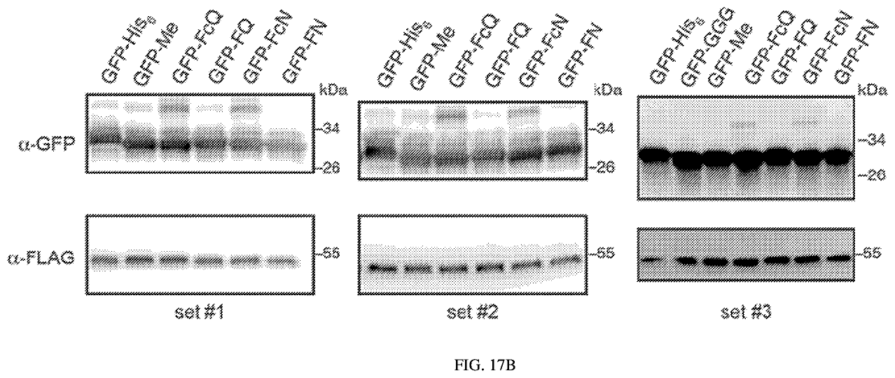

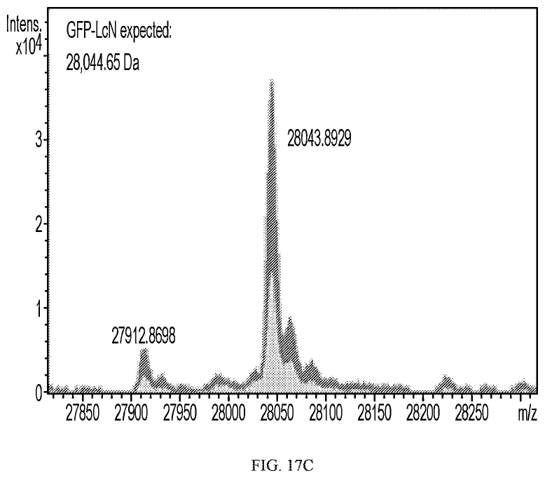

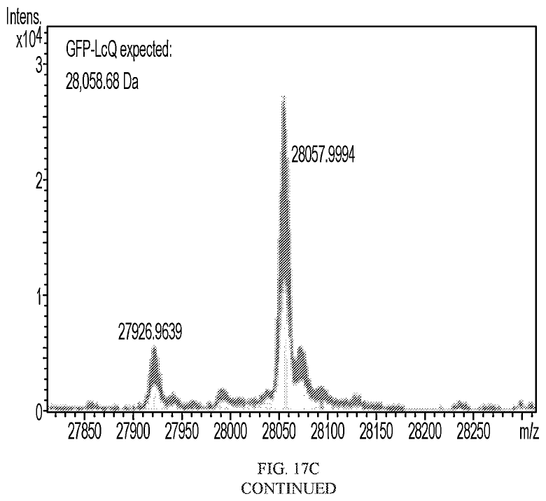

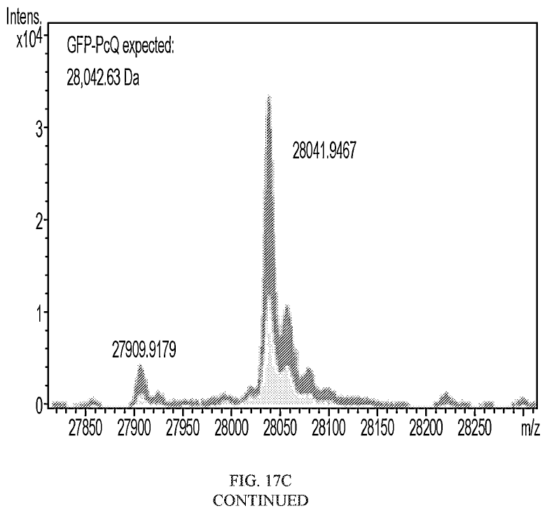

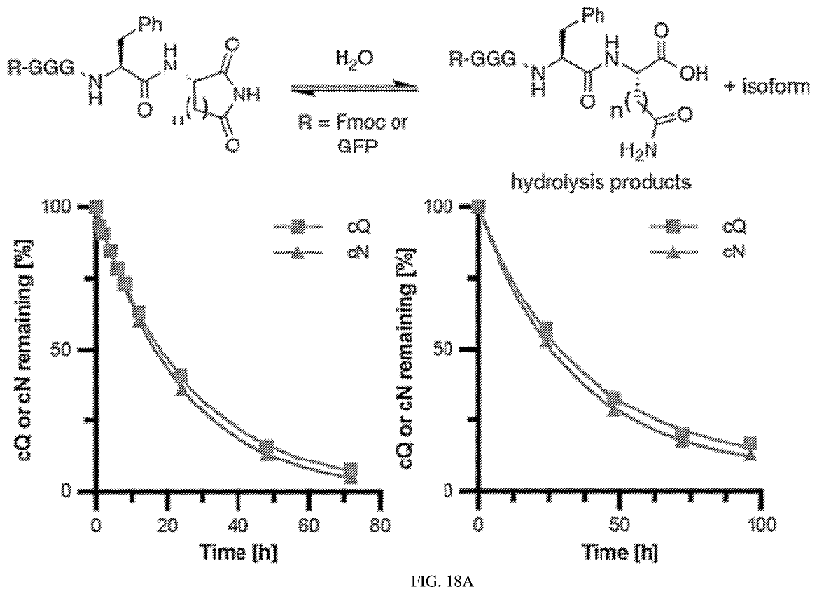

Next, the sequence specificity of the C-terminal cyclic imide degron and its activity across cell lines and species was evaluated. Tailoring of the C-terminus of GFP with PcQ, LcQ, or LcN significantly accelerated depletion of GFP relative to GFP-His6 in HEK293T cells, which was rescued by lenalidomide competition (FIG. 15G and FIG. 17C). Depletion of GFP dependent on C-terminal cyclic imides was additionally observed across cell lines (Jurkat cells) and species [mouse embryonic fibroblast cells (MEF), FIG. 15H and FIG. 15I. These data indicate that recognition of the C-terminal cyclic imide degron by CRBN is conserved across cell types and species and that CRBN flexibly accepts various amino acid residues adjacent to the cyclic imide.

### The C-Terminal Cyclic Imide Degron is Transferrable to Other Degraders and Proteins

If thalidomide is mimicking the C-terminal cyclic imide degron as in the second model, it was hypothesized that the degron would readily substitute thalidomide in a separate small molecule degrader. The thalidomide moiety of the degrader dFKBP-130 was substituted with FcQ to afford dFKBP-FcQ (FIG. 4E). Both dFKBP-1 and dFKBP-FcQ degraded the target protein FKBP12 over a similar dose-dependent concentration range (FIG. 4F), and degradation was inhibited upon co-treatment with lenalidomide or Boc-FcQ (FIG. 4G). The rate of FKBP12 degradation was slightly slower with dFKBP-FcQ and was completed within 18 h (FIG. 12F). These data indicate that the dipeptide FcQ is a transferrable chemical motif when incorporated to a bifunctional small molecule for degradation of a target protein, which is a consistent with previously described degrons. The pomalidomide moiety of a third degrader, dCDK6-Pom (N. A. Anderson et al., Bioorg Med Chem Lett 30, 127106 (2020); B. Jiang et al., Angew Chem Int Ed Engl 58, 6321-6326 (2019)) was also readily substituted with FcQ to afford the active CDK6 degrader, dCDK6-FcQ (FIGS. 4H-4J). These data indicate that the dipeptide FcQ is a transferrable chemical motif when incorporated into a bifunctional small molecule for degradation of a target protein. Furthermore, the cyclic imides were transferrable degrons that promoted ubiquitylation and degradation of GST-FKBP12 as a separate substrate at the thalidomide binding domain of CRBN in vitro and in cells (FIG. 5E, FIGS. 13C-13D). Therefore, cyclic imides are genuine degrons of CRBN that promote degradation of proteins bearing this modification at the C-terminus in cells.

### C-Terminal Cyclic Imides are Overlooked Post-Translational Modifications

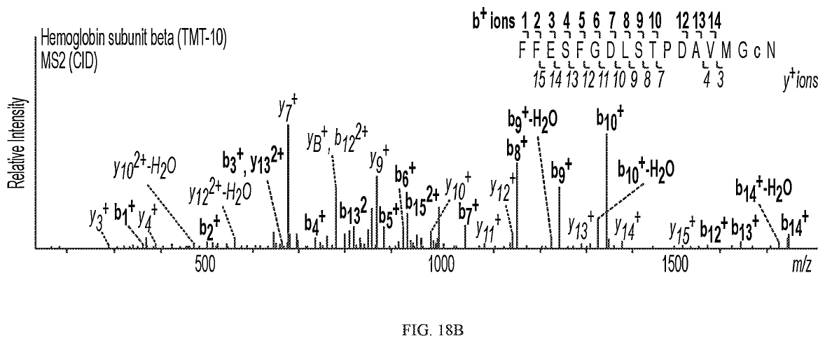

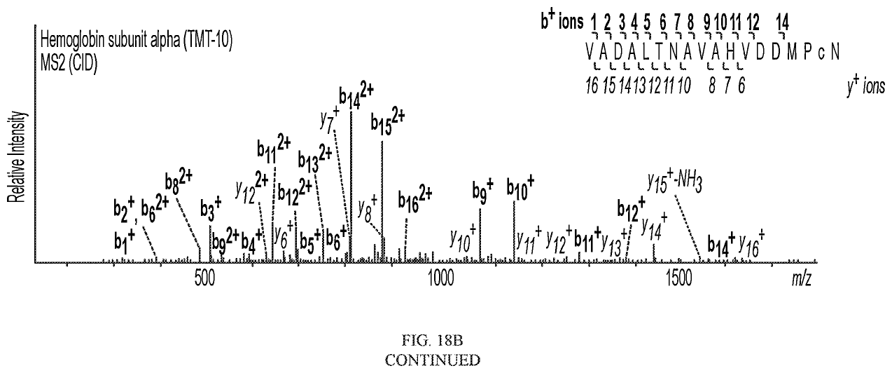

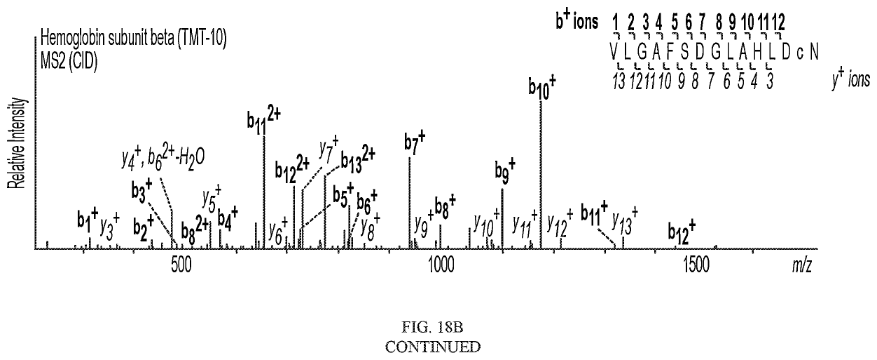

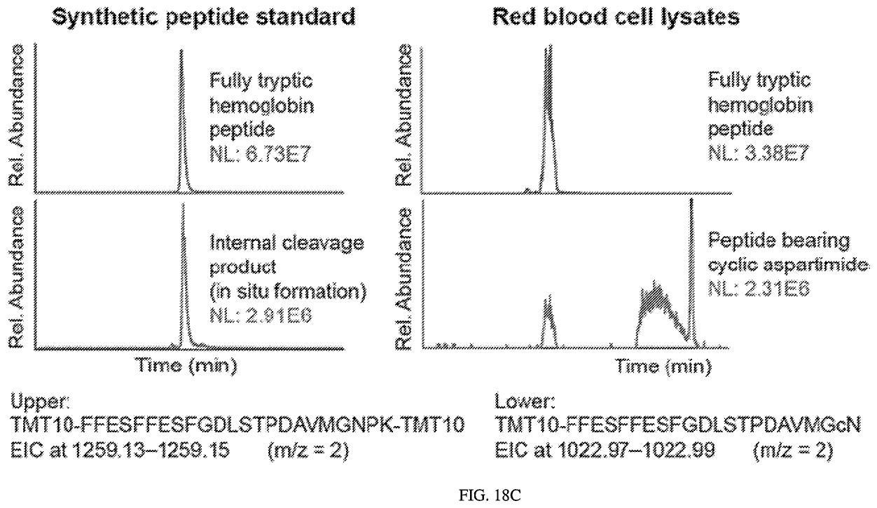

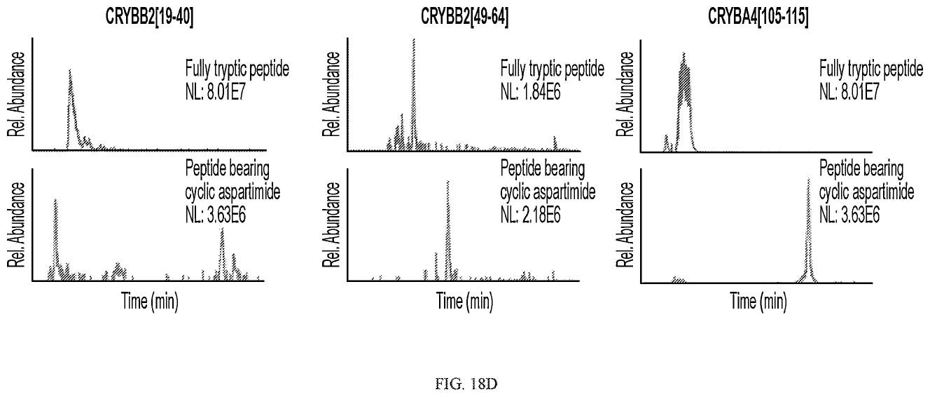

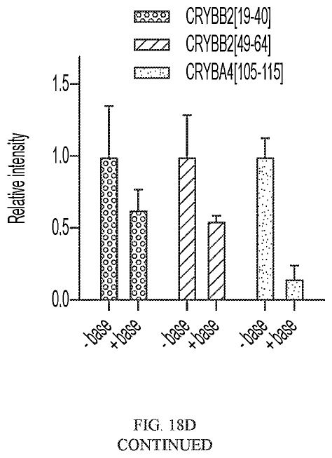

Finally, evidence for the natural occurrence of cyclic imide PTMs in the proteome was sought. Cyclic imides are formed spontaneously during Asn or Gln deamidation and an analogous process consisting of nucleophilic attack from the amide side chain results in protein cleavage and reveals the degron for CRBN (FIG. 5G). Although cyclic imides are presumed to undergo subsequent hydrolysis, the hydrolysis of C-terminal cyclic imides was examined on short peptides in vitro and found that their half-lives at 37° C. in PBS (t_{1/2} cQ=18.4 h, t_{1/2} cN=16.7 h) was well within the degradation timeframe of most known substrates of CRBN induced by the IMiDs (2-8 h in cells, FIG. 13E and FIG. 18A). The cyclic imide half-life at 37° C. additionally appears to increase when the degron is appended to the C-terminus of a protein (t_{1/2} GFP-FcQ=24.9 h, t_{1/2} GFP-FcN=23.0 h at 37° C., FIG. 13F and FIG. 18A).

The ready formation of these modifications via intramolecular cyclization and their relatively long-lived half-lives indicates their availability for recognition by CRBN and that the actual prevalence of C-terminal cyclic imide PTMs in the cellular proteome may be higher than previously recognized due to their depletion by CRBN. To detect these modifications in human proteomes, a meta-analysis was performed for cyclic imides using global proteomics datasets of the NCI7 cell line panel and six primary human tissue samples.³⁷⁻⁴³ Several hundred proteins with over one thousand unique sites of cN or cQ modification across these datasets were identified (FIGS. 5H-5I). Notably, the breast, brain, liver, and kidney proteomes appear to carry more of these modifications than oral or colon proteomes. The identification of over a thousand cN and cQ modification sites dramatically expands the map of these modifications in the human proteome and implies that they are more prevalent than previously understood.

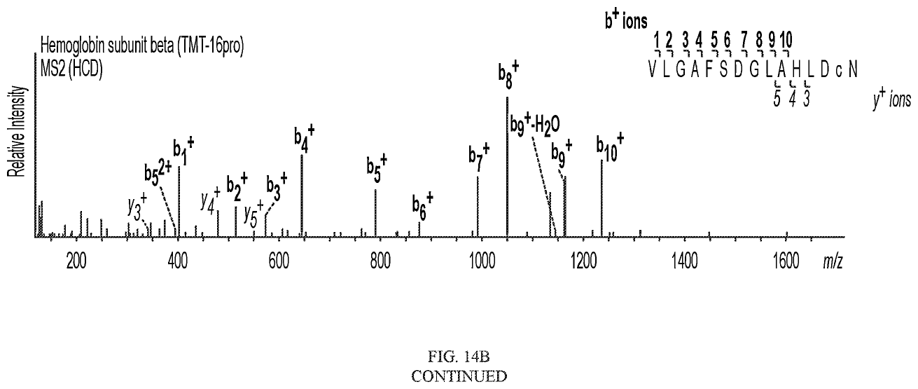

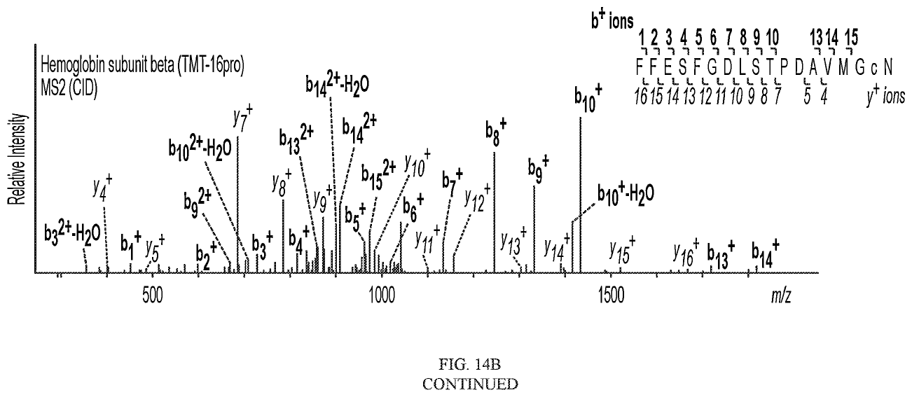

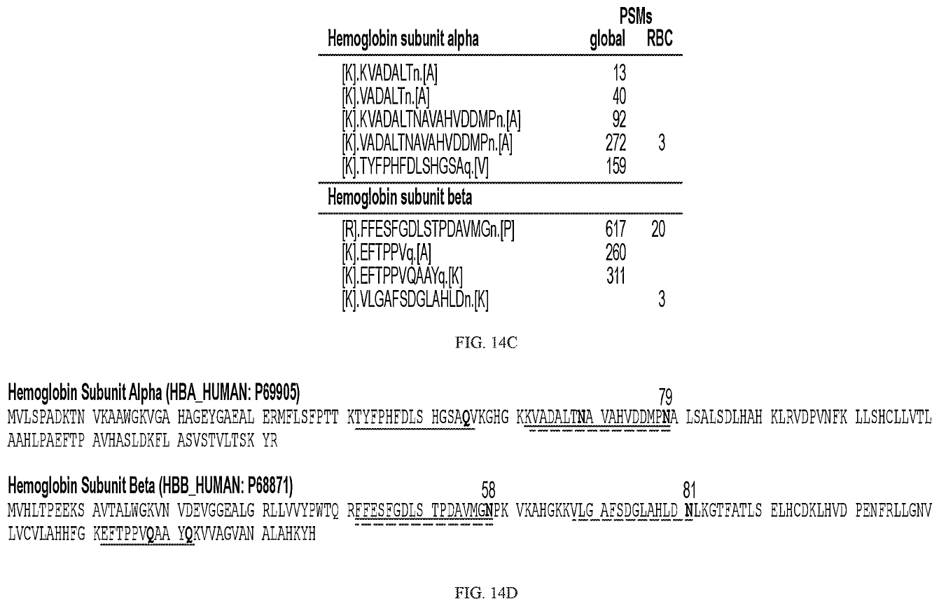

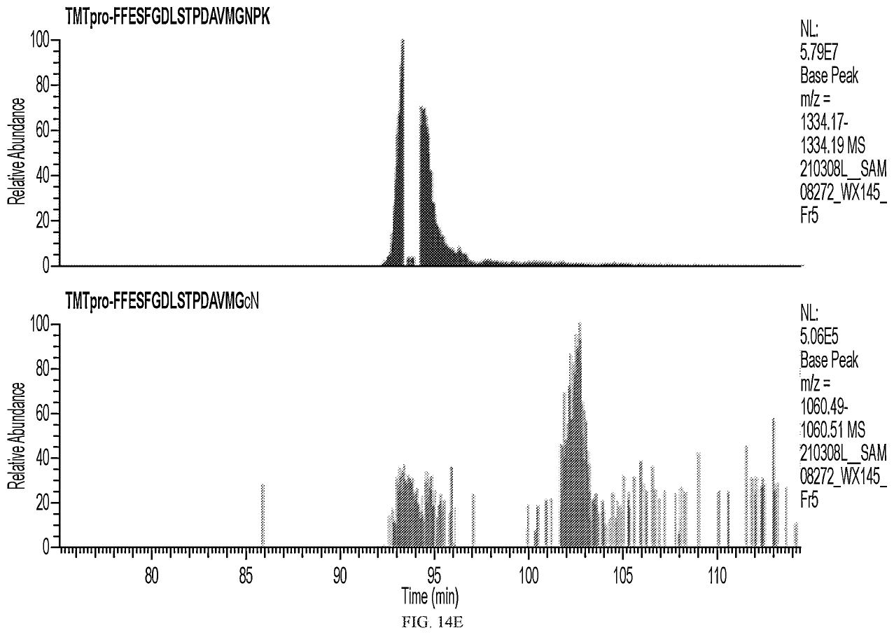

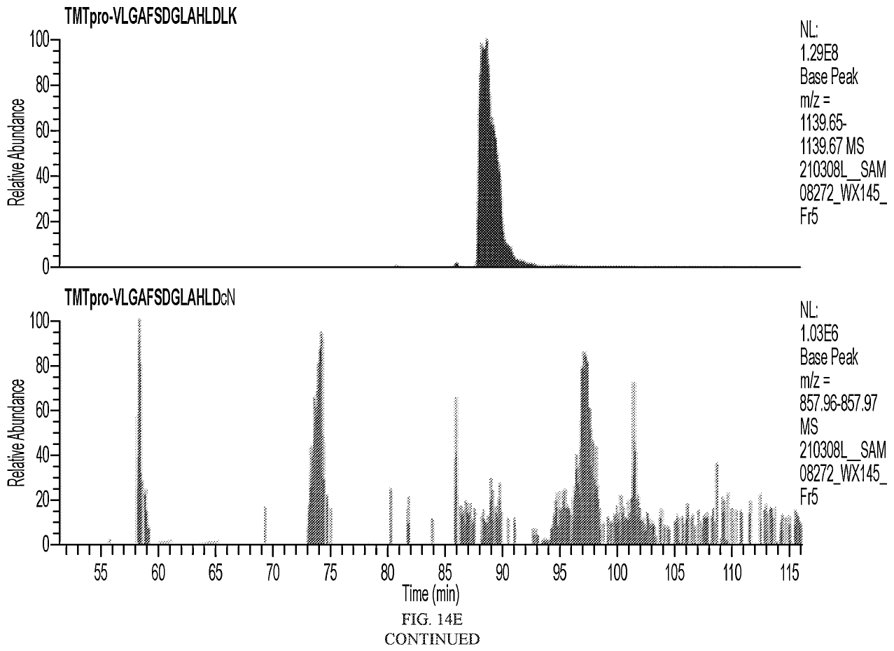

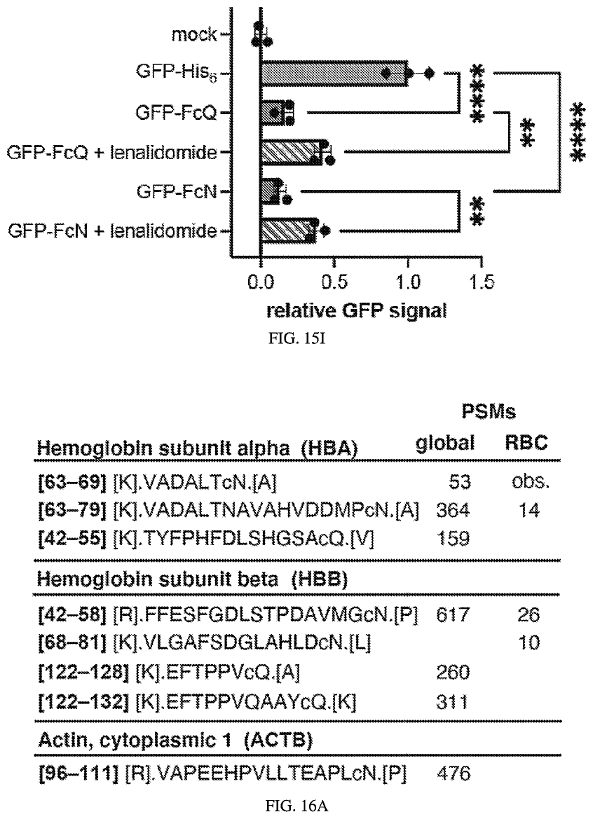

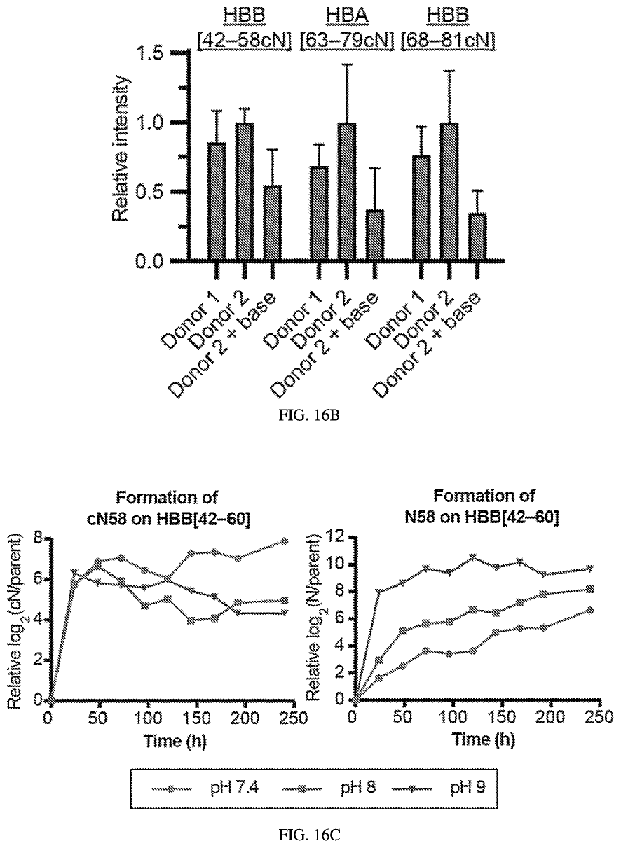

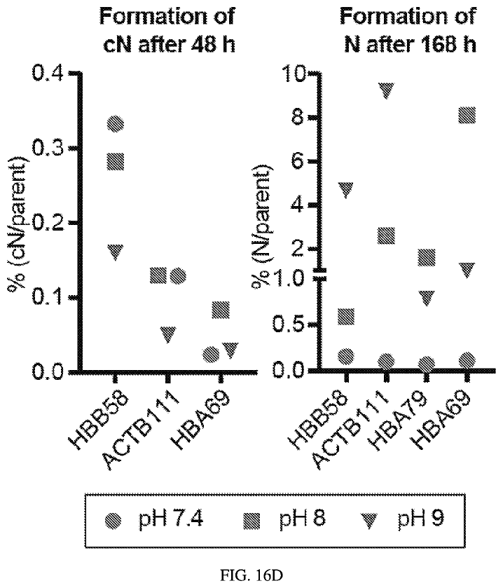

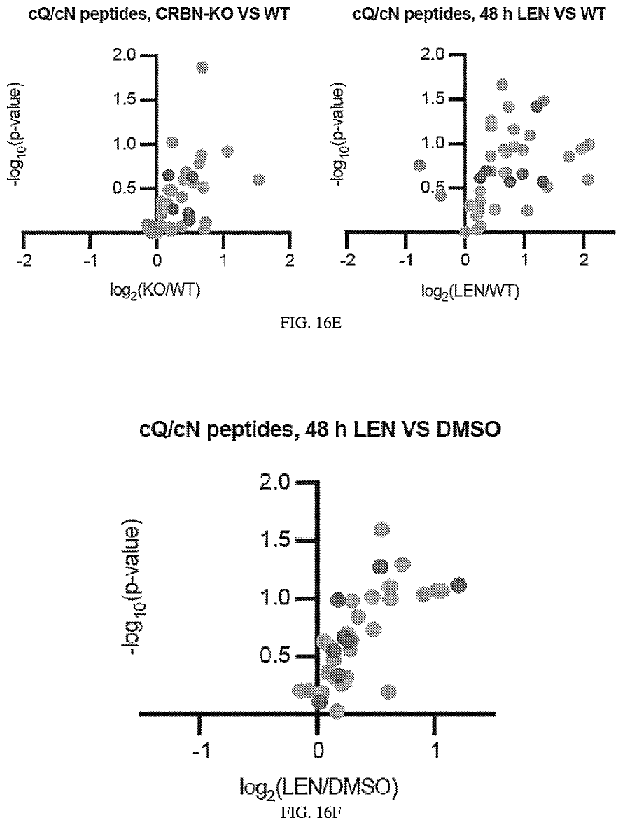

It was noted that C-terminal imides derived from hemoglobin subunits alpha and beta were two of the most frequently observed proteins by spectral counting in the analyses, with a total of eight unique cN or cQ modification sites identified across the global proteomics datasets. Hemoglobin globally expressed across tissue types, but is a particularly highly abundant protein in red blood cells, which have a lifespan of approximately 120 days, but do not express CRBN.⁴⁴ Therefore, cyclic imides formed on hemoglobin during protein aging may accumulate and not be degraded in vivo. Indeed, CRBN was not observed in red blood cells by proteomics or Western blot (FIG. 14A). To confirm the existence of cN and cQ modifications in red blood cells, cell lysates were digested from two healthy donors with trypsin for 1 h at 47° C. Three unique peptides bearing a C-terminal cN modification were observed (for representative spectra see FIG. 5J, FIG. 14B, and FIG. 18B). Two of the three modified peptides and cyclic imide sites observed from red blood cell lysates overlapped with those identified by the analysis of global proteomic datasets (FIGS. 14C-14D). All three peptides were fully responsive to base-treatment performed prior to mass spectrometry analysis, which induces the destruction of C-terminal cyclic imide via hydrolysis. These peptides were observed at retention times independent of the fully tryptic peptide, indicating that these species are present in the sample and not formed during mass spectrometry analysis (FIG. 14E, FIG. 16B, and FIG. 18C).

To further extend the observation of C-terminal cyclic imide modified proteins in aged protein samples, bovine eye lenses were obtained as crystallins, the major proteins of the lens, which are known to undergo deamidation to afford cyclic imide modifications during aging, leading to their structural instability and aggregation (T. Takata, et al. Protein Sci 17, 1565-1575 (2008); P. A. Wilmarth et al. J Proteome Res 5, 2554-2566 (2006)). Twenty-two modification sites were mapped, mostly on beta-crystallins, that were responsive to base treatment. In all cases, these modified peptides were differentiable from the corresponding tryptic peptides by their different retention times (FIG. 18D). The identification of over a thousand cN and cQ modification sites across proteins in human tissues, red blood cells, and bovine eye lenses dramatically expands the map of these modifications in the human proteome and implies that they are more prevalent than previously understood.

C-Terminal Cyclic Imides Readily Form on Peptides and Endogenous Proteins that are Regulated by CRBN

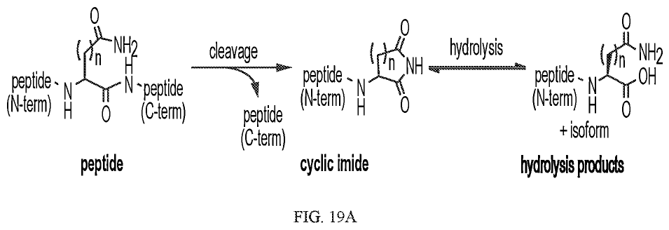

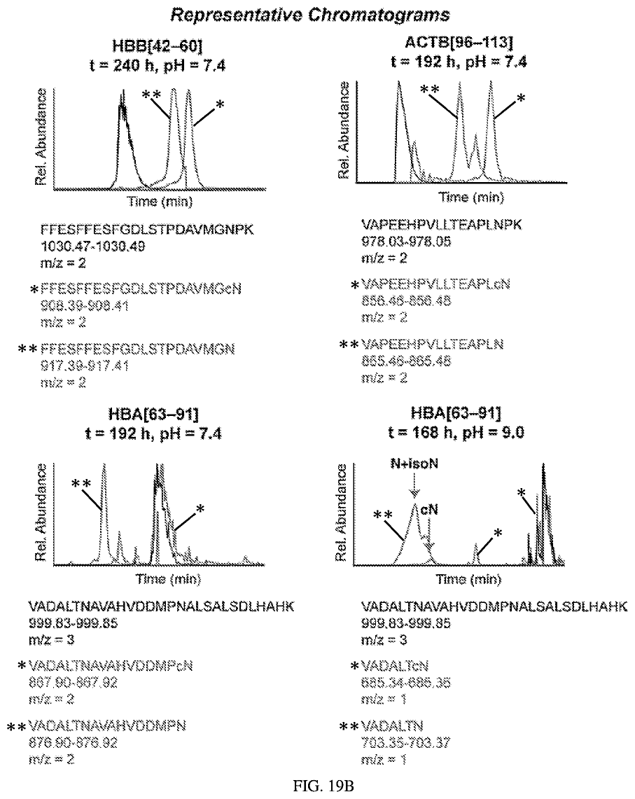

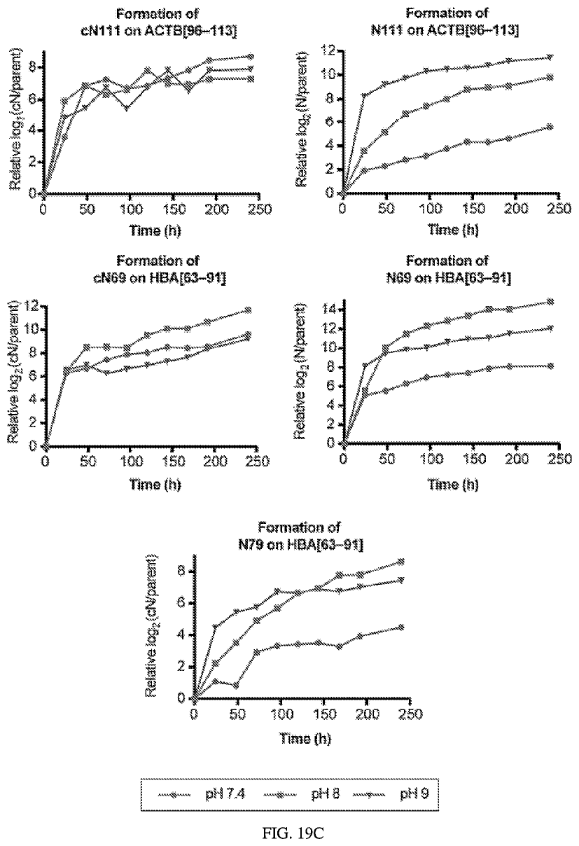

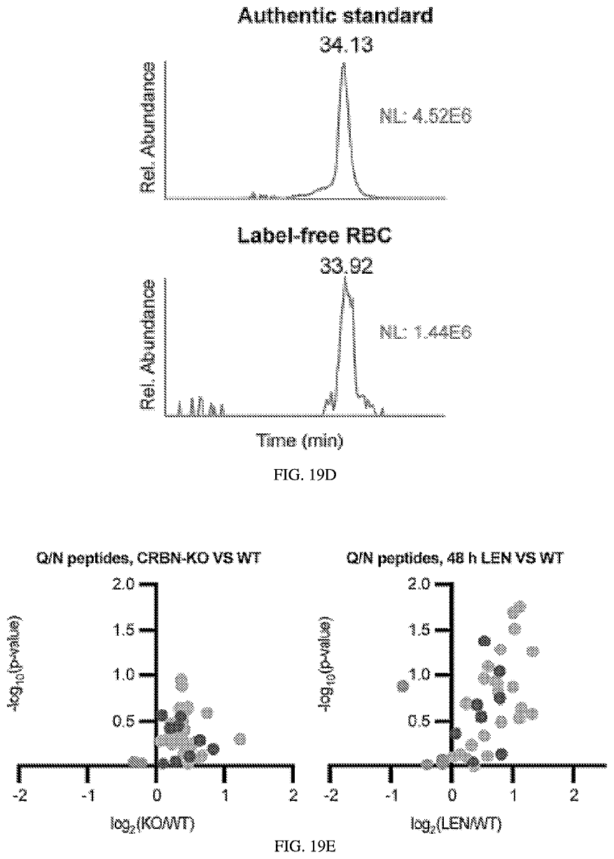

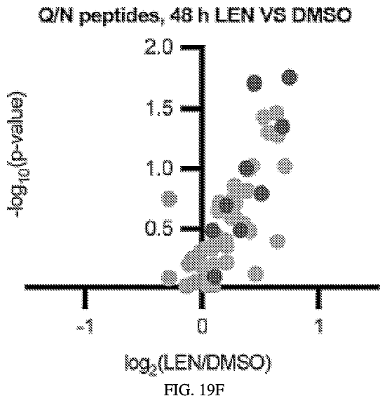

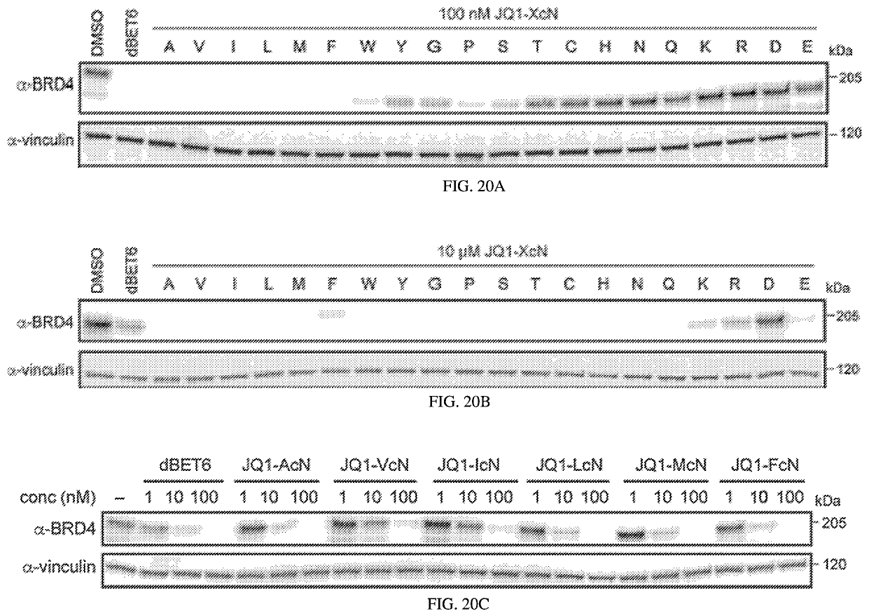

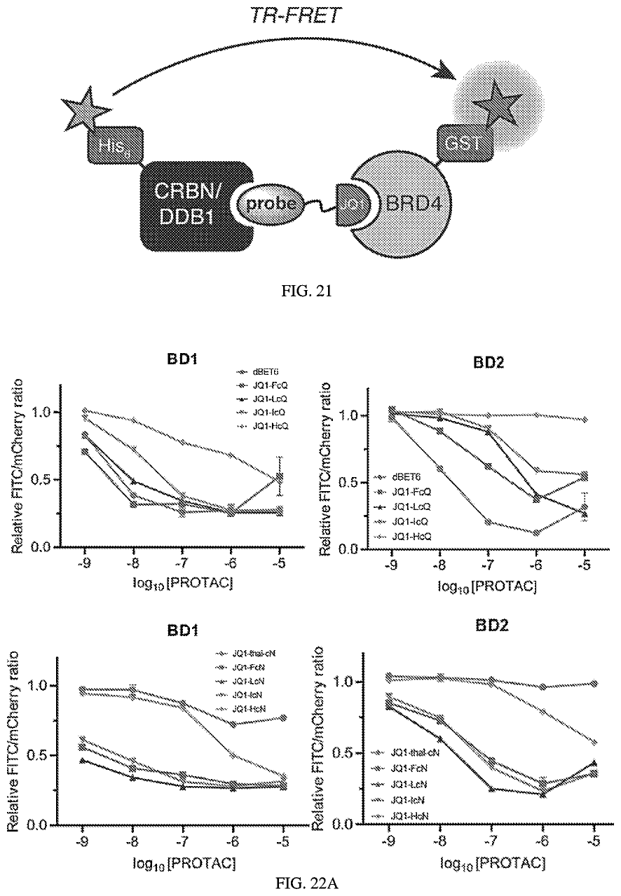

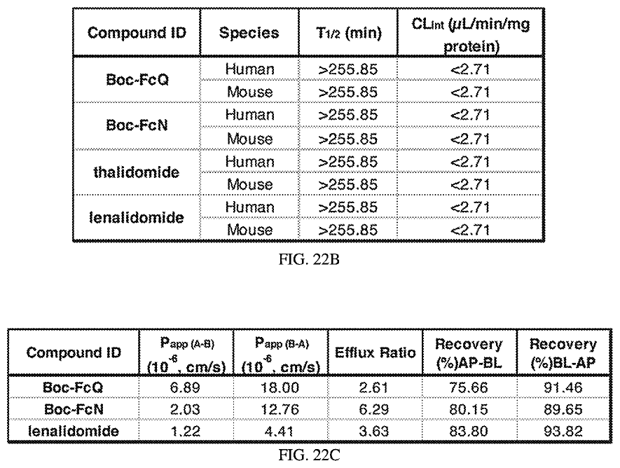

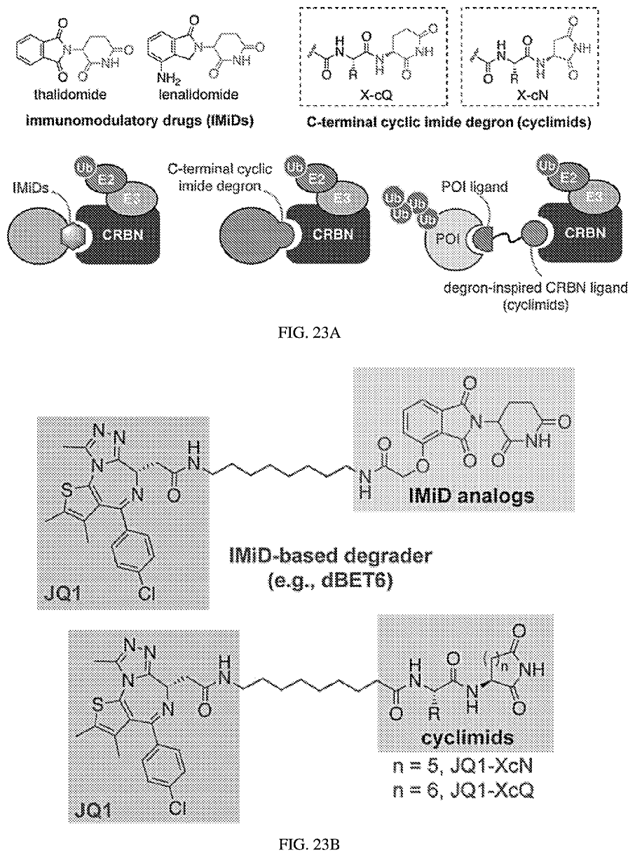

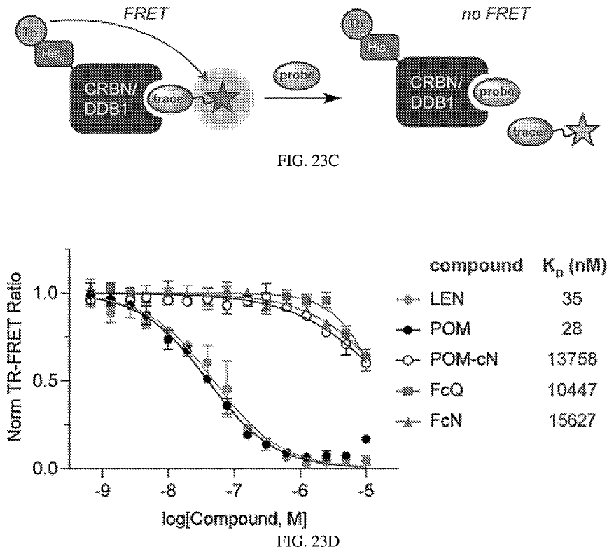

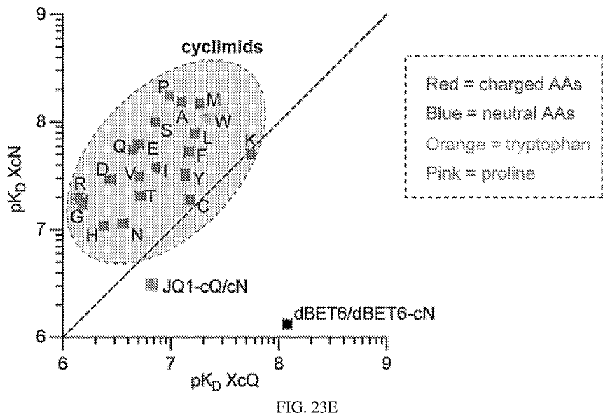

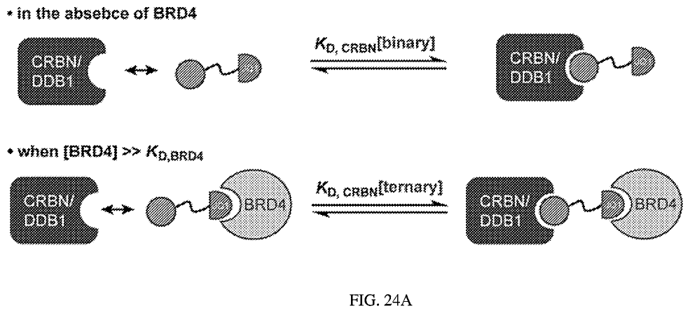

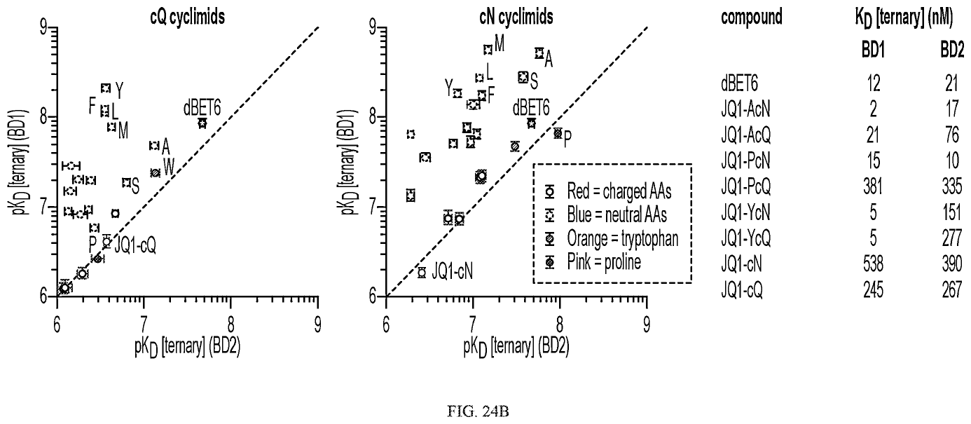

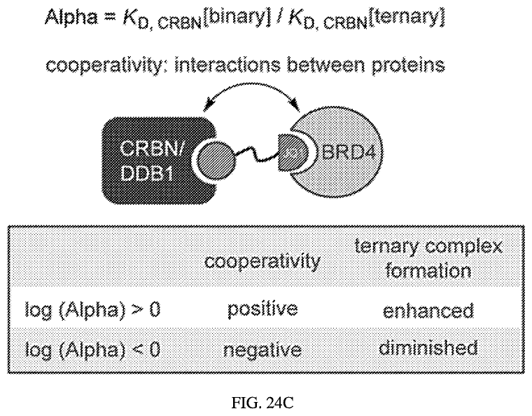

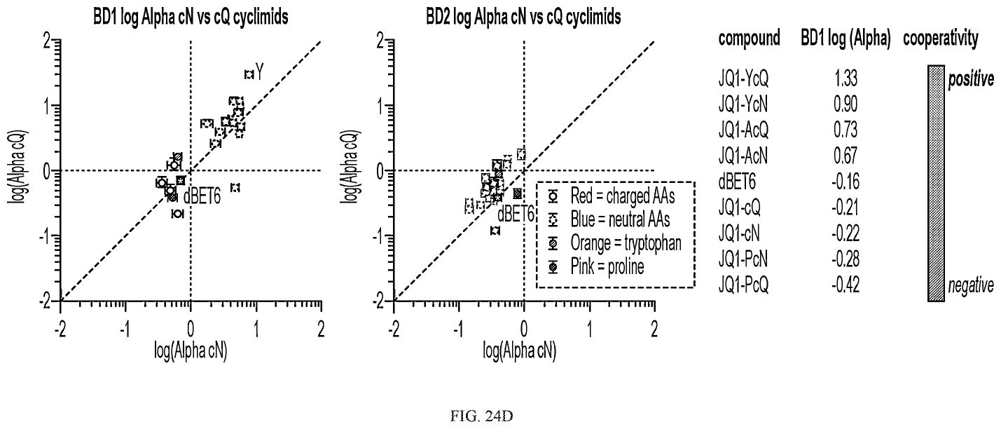

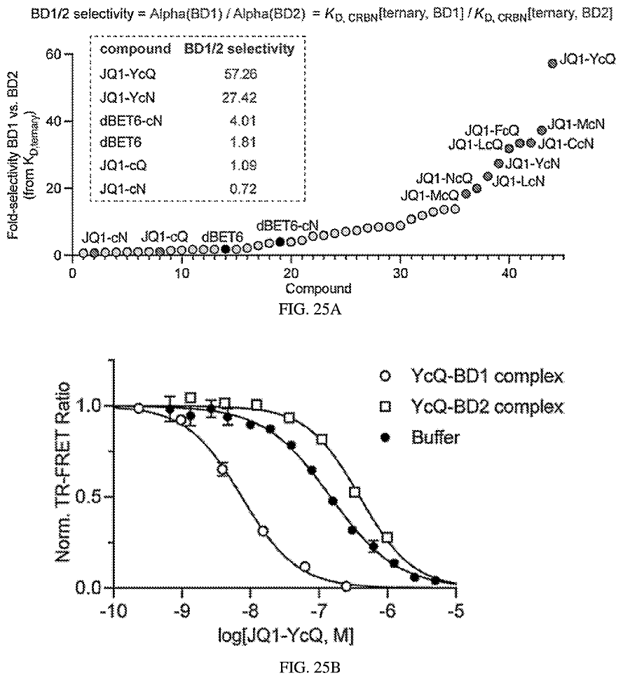

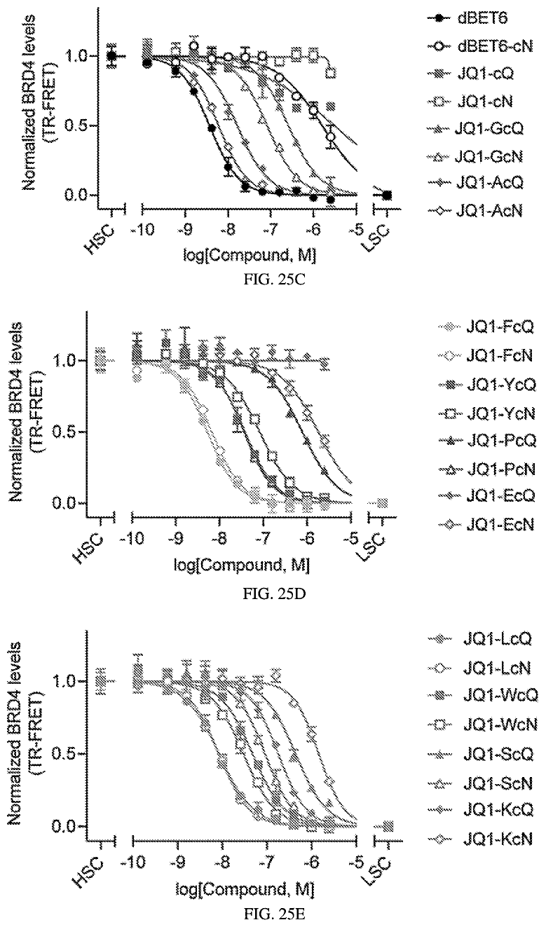

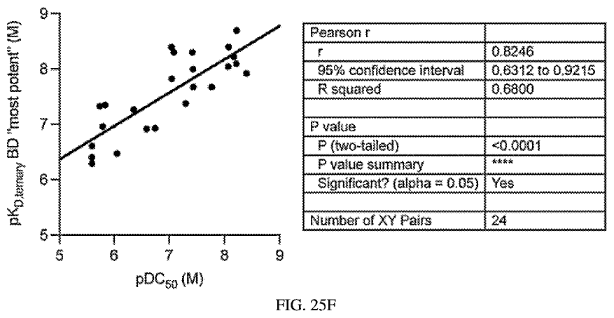

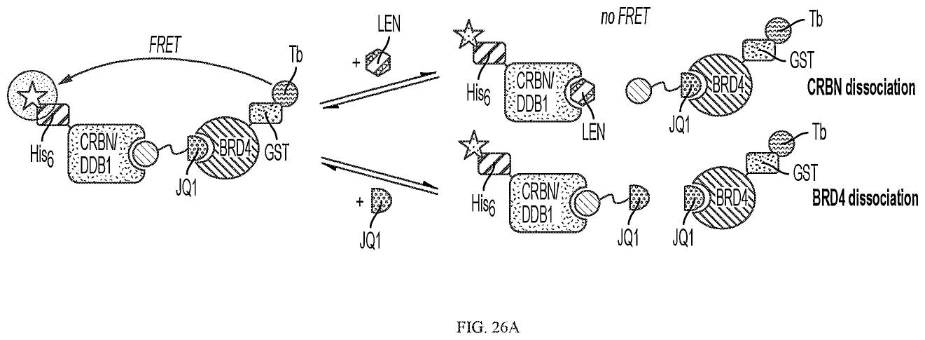

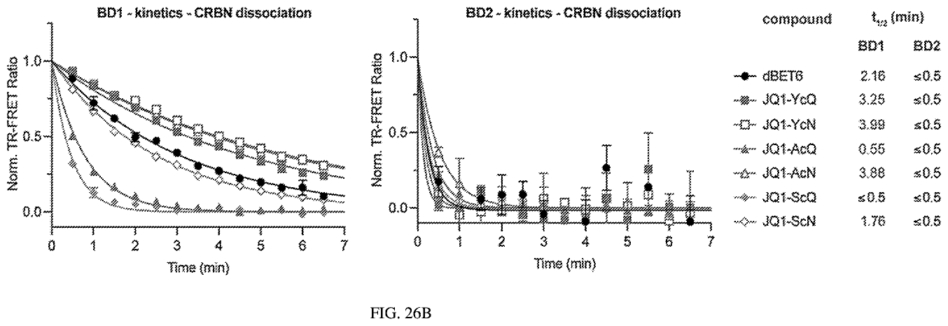

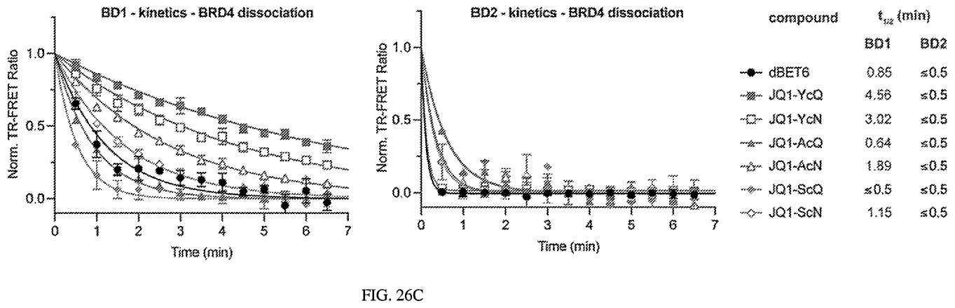

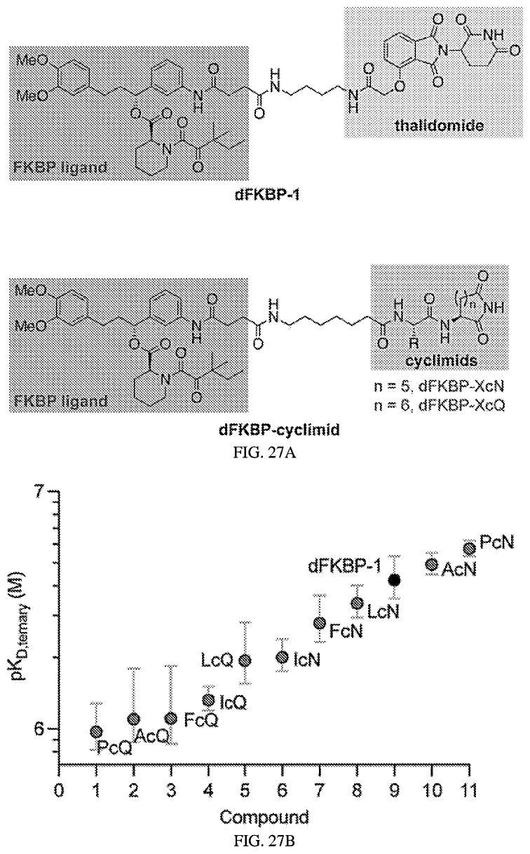

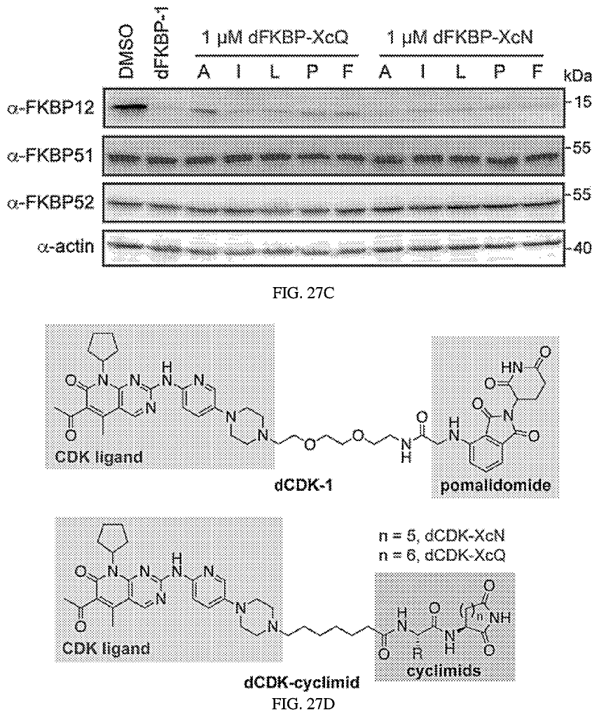

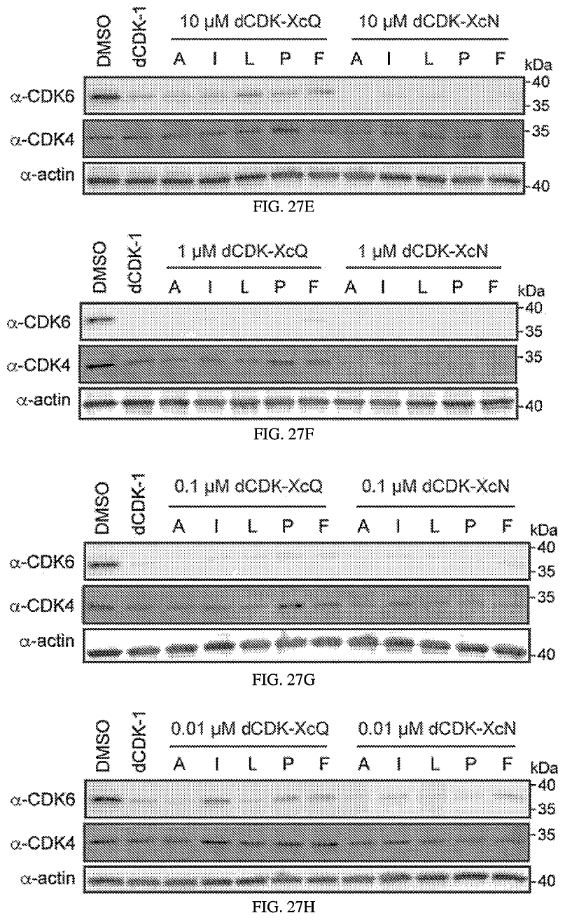

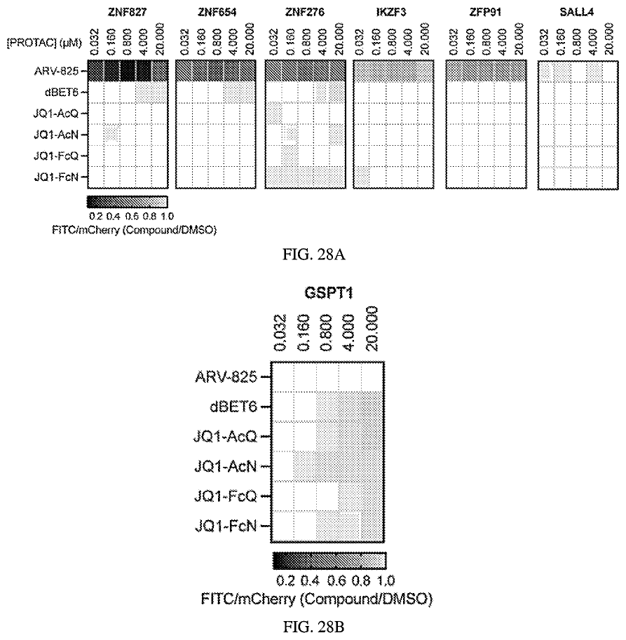

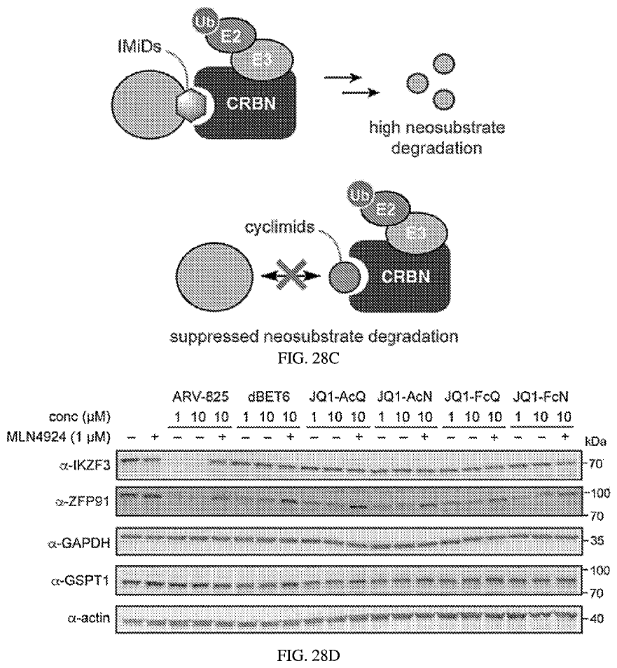

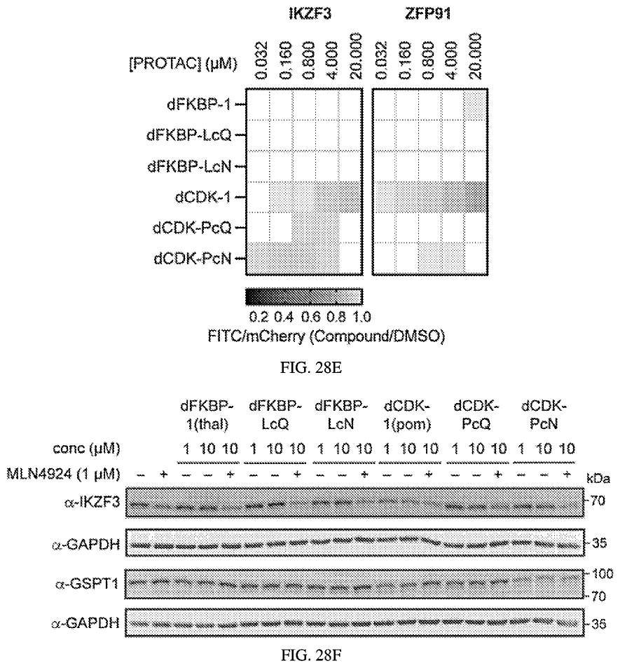

Enthused by the detection of C-terminal cyclic imides across various biological sources by global proteomics, verification of the ready formation of C-terminal cyclic imide modifications was sought with three synthetic peptides from proteins bearing four of the most frequently observed modification sites: HBB[42-60], HBA[63-91], and ACTB[96-113] (FIG. 16A). These peptides were monitored for internal cleavage and formation of the C-terminal cyclic imide, followed by downstream hydrolysis at 37° C., over a pH range of 7.4-9.0 (FIG. 19A). The extracted ion chromatograms for the parent peptide, the cyclic imide-bearing fragment, and its hydrolyzed products showed distinct retention times, with the exception of the cyclic imide fragment at HBA cN79, although the hydrolysis product was readily observed (FIG. 19B). The spontaneous formation of the cyclic imide fragment was observed from each peptide after 24 h and appeared to plateau after 48 h. In contrast, for all four sites, the abundance of the hydrolyzed forms of the cyclic imide fragment continuously increased over 10 d in a pH-dependent manner (FIGS. 16C-16D, FIG. 19C). The synthetic peptide HBB[42-60] affords HBB[42-cN58] after incubation, which eluted at the same retention time as the cyclic imide fragment observed from RBCs on liquid chromatography, further validating the presence of these modifications in biological samples (FIG. 19D). Although for any individual peptide sequence, the C-terminal cyclic imide concentration plateaued at a low level, the gradual increase of the corresponding hydrolyzed fragments suggests that these undesired fragments will accumulate significantly over time if there is no mechanism for removal of protein fragments bearing the C-terminal cyclic imide PTM.

The global increase in C-terminal cyclic imide modifications observed across a range of proteins following loss or competitive inhibition of CRBN would align with a protein damage or aging mechanism that generates these modifications, which are recognized for ubiquitylation and degradation by CRL4^{CRBN}. Thus, it was evaluated whether the presence of the PTM on endogenous substrates would promote removal of the proteins by CRBN-dependent degradation. To investigate whether the level of C-terminal cyclic imides across proteins are dependent on availability of the thalidomide-binding domain of CRBN, a quantitative proteomics experiment was performed to compare the global proteome obtained from HEK293T cells before and after CRBN knockout by CRISPR/Cas9 or treatment with lenalidomide using an exogenously added protein (GFP) as an internal control. It was found that 39 unique peptides bearing C-terminal cyclic 5 imides and observed the majority of these modified peptides increase when CRBN is knocked out or the thalidomide binding domain of CRBN is inhibited by lenalidomide (FIG. 16E). A similar increase in 34 out of 36 peptides bearing the C-terminal cyclic imide PTM was identified in MM.1S cells after treatment with lenalidomide (FIG. 16F). Peptides bearing C-terminal asparagine and glutamine also increase in both cell lines upon CRBN knockout or inhibition, consistent with a model where blockade of degradation of substrates bearing the C-terminal imide allows time for the modifications to hydrolyze (FIGS. 19E-19F). Thus, the thalidomide binding domain of CRBN promotes the ubiquitylation and eventual degradation of endogenous substrates bearing C-terminal cyclic imides that form in a surprisingly rapid and substrate-indiscriminate manner to prevent the unwanted accumulation of these fragments and their hydrolysis products over time. These data, combined with the conservation of the thalidomide-binding domain of CRBN across species, 35 implies that C-terminal cyclic imides are common PTMs that represent a core degron recognized by CRBN for substrate degradation.

### REFERENCES

1. T. Ito et al., Identification of a Primary Target of Thalidomide Teratogenicity. Science 327, 1345-1350 (2010).
2. G. Lu et al., The Myeloma Drug Lenalidomide Promotes the Cereblon-Dependent Destruction of Ikaros Proteins. Science 343, 305-309 (2014).
3. J. Krönke et al., Lenalidomide Causes Selective Degradation of IKZF1 and IKZF3 in Multiple Myeloma Cells. Science 343, 301-305 (2014).
4. J. Kronke et al., Lenalidomide induces ubiquitination and degradation of CK1[agr] in del(5q) MDS. Nature 523, 183-188 (2015).
5. M. E. Matyskiela et al., A novel cereblon modulator recruits GSPT1 to the CRL4(CRBN) ubiquitin ligase. Nature 535, 252-257 (2016).
6. K. A. Donovan et al., Thalidomide promotes degradation of SALL4, a transcription factor implicated in Duane Radial Ray syndrome. eLife 7, e38430 (2018).
7. M. E. Matyskiela et al., SALL4 mediates teratogenicity as a thalidomide-dependent cereblon substrate. Nat Chem Biol 14, 981-987 (2018).
8. T. Asatsuma-Okumura et al., p63 is a cereblon substrate involved in thalidomide teratogenicity. Nat Chem Biol 15, 1077-1084 (2019).
9. A. Bachmair, D. Finley, A. Varshavsky, In vivo half-life of a protein is a function of its amino-terminal residue. Science 234, 179-186 (1986).
10. I. Koren et al., The Eukaryotic Proteome Is Shaped by E3 Ubiquitin Ligases Targeting C-Terminal Degrons. Cell 173, 1622-1635 e1614 (2018).
11. H.-C. Lin et al., C-Terminal End-Directed Protein Elimination by CRL2 Ubiquitin Ligases. Mol Cell 70, 602-613.e603 (2018).
12. P. H. Maxwell et al., The tumour suppressor protein VHL targets hypoxia-inducible factors for oxygen-dependent proteolysis. Nature 399, 271-275 (1999).
13. W. M. Gray, S. Kepinski, D. Rouse, O. Leyser, M. Estelle, Auxin regulates SCFTIR1-dependent degradation of AUX/IAA proteins. Nature 414, 271-276 (2001).
14. E. S. Fischer et al., Structure of the DDB1-CRBN E3 ubiquitin ligase in complex with thalidomide. Nature 512, 49-53 (2014).
15. D. Del Prete, R. C. Rice, A. M. Rajadhyaksha, L. D'Adamio, Amyloid Precursor Protein (APP) May Act as a Substrate and a Recognition Unit for CRL4^{CRBN} and Stub1 E3 Ligases Facilitating Ubiquitination of Proteins Involved in Presynaptic Functions and Neurodegeneration. J Biol Chem 291, 17209-17227 (2016).
16. P. P. Chamberlain et al., Structure of the human Cereblon-DDB1-lenalidomide complex reveals basis for responsiveness to thalidomide analogs. Nat Struct Mol Biol 21, 803-809 (2014).
17. M. D. Hartmann et al., Thalidomide mimics uridine binding to an aromatic cage in cereblon. J Struct Biol 188, 225-232 (2014).
18. I. Boichenko, S. Deiss, K. Bar, M. D. Hartmann, B. Hernandez Alvarez, A FRET-Based Assay for the Identification and Characterization of Cereblon Ligands. J Med Chem 59, 770-774 (2016).
19. S. Schilling et al., Glutaminyl cyclase inhibition attenuates pyroglutamate Abeta and Alzheimer's disease-like pathology. Nat Med 14, 1106-1111 (2008).
20. T. Geiger, S. Clarke, Deamidation, isomerization, and racemization at asparaginyl and aspartyl residues in peptides. Succinimide-linked reactions that contribute to protein degradation. J Biol Chem 262, 785-794 (1987).
21. C. E. Voorter, W. A. de Haard-Hoekman, P. J. van den Oetelaar, H. Bloemendal, W. W. de Jong, Spontaneous peptide bond cleavage in aging alpha-crystallin through a succinimide intermediate. J Biol Chem 263, 19020-19023 (1988).
22. R. Tyler-Cross, V. Schirch, Effects of amino acid sequence, buffers, and ionic strength on the rate and mechanism of deamidation of asparagine residues in small peptides. J Biol Chem 266, 22549-22556 (1991).
23. T. V. Brennan, S. Clarke, Effect of adjacent histidine and cysteine residues on the spontaneous degradation of asparaginyl- and aspartyl-containing peptides. Int J Pept Protein Res 45, 547-553 (1995).
24. H. Paulus, Protein splicing and related forms of protein autoprocessing. Annu Rev Biochem 69, 447-496 (2000).
25. K. V. Mills, J. S. Manning, A. M. Garcia, L. A. Wuerdeman, Protein splicing of a Pyrococcus abyssi intein with a C-terminal glutamine. J Biol Chem 279, 20685-20691 (2004).
26. M. Q. Xu et al., Protein splicing: an analysis of the branched intermediate and its resolution by succinimide formation. EMBO J 13, 5517-5522 (1994).
27. Y. Shao, M. Q. Xu, H. Paulus, Protein splicing: characterization of the aminosuccinimide residue at the carboxyl terminus of the excised intervening sequence. Biochemistry 34, 10844-10850 (1995).
28. S. Frutos, M. Goger, B. Giovani, D. Cowburn, T. W. Muir, Branched intermediate formation stimulates peptide bond cleavage in protein splicing. Nat Chem Biol 6, 527-533 (2010).
29. K. M. Sakamoto et al., Protacs: chimeric molecules that target proteins to the Skp1-Cullin-F box complex for ubiquitination and degradation. Proceedings of the National Academy of Sciences of the United States of America 98, 8554-8559 (2001).
30. G. E. Winter et al., Phthalimide conjugation as a strategy for in vivo target protein degradation. Science 348, 1376-1381 (2015).
31. J. Lu et al., Hijacking the E3 Ubiquitin Ligase Cereblon to Efficiently Target BRD4. Chem Biol 22, 755-763 (2015).
32. R. P. Nowak et al., Plasticity in binding confers selectivity in ligand-induced protein degradation. Nat Chem Biol 14, 706-714 (2018).
33. T. V. Nguyen et al., Glutamine Triggers Acetylation-Dependent Degradation of Glutamine Synthetase via the Thalidomide Receptor Cereblon. Mol Cell 61, 809-820 (2016).
34. B. E. Smith et al., Differential PROTAC substrate specificity dictated by orientation of recruited E3 ligase. Nat Commun 10, 131 (2019).
35. A. N. Lupas, H. Zhu, M. Korycinski, The thalidomide-binding domain of cereblon defines the CULT domain family and is a new member of the beta-tent fold. PLOS computational biology 11, e1004023 (2015).
36. Q. L. Sievers et al., Defining the human C2H₂ zinc finger degrome targeted by thalidomide analogs through CRBN. Science 362, (2018).
37. B. Zhang et al., Proteogenomic characterization of human colon and rectal cancer. Nature 513, 382-387 (2014).
38. P. Mertins et al., Proteogenomics connects somatic mutations to signalling in breast cancer. Nature 534, 55-62 (2016).
39. T. W. Chen et al., APOBEC3A is an oral cancer prognostic biomarker in Taiwanese carriers of an APOBEC deletion polymorphism. Nat Commun 8, 465 (2017).
40. D. J. Clark et al., Evaluation of NCI-7 Cell Line Panel as a Reference Material for Clinical Proteomics. Journal of proteome research 17, 2205-2215 (2018).
41. Q. Gao et al., Integrated Proteogenomic Characterization of HBV-Related Hepatocellular Carcinoma. Cell 179, 561-577 e522 (2019).
42. D. J. Clark et al., Integrated Proteogenomic Characterization of Clear Cell Renal Cell Carcinoma. Cell 179, 964-983 e931 (2019).
43. L. B. Wang et al., Proteogenomic and metabolomic characterization of human glioblastoma. Cancer Cell, (2021).
44. A. H. Bryk, J. R. Wisniewski, Quantitative Analysis of Human Red Blood Cell Proteome. Journal of proteome research 16, 2752-2761 (2017).
45. D. L. Buckley et al., Targeting the von Hippel-Lindau E3 Ubiquitin Ligase Using Small Molecules To Disrupt the VHL/HIF-1a Interaction. J Am Chem Soc 134, 4465-4468 (2012).
46. A. Varshavsky, N-degron and C-degron pathways of protein degradation. Proc Natl Acad Sci 116, 358-366 (2019).
47. M. Ishoey et al., Translation Termination Factor GSPT1 Is a Phenotypically Relevant Off-Target of Heterobifunctional Phthalimide Degraders. ACS Chem Biol 13, 553-560 (2018).
48. Y. X. Zhu, K. M. Kortuem, A. K. Stewart, Molecular mechanism of action of immune-modulatory drugs thalidomide, lenalidomide and pomalidomide in multiple myeloma. Leuk. Lymphoma 54, 683-687 (2013).
49. A. B. Robinson, C. J. Rudd, Deamidation of glutaminyl and asparaginyl residues in peptides and proteins. Curr Top Cell Regul 8, 247-295 (1974).
50. L. Takemoto, D. Boyle, Deamidation of specific glutamine residues from alpha-A crystallin during aging of the human lens. Biochemistry 37, 13681-13685 (1998).
51. Li, M.; Tao, Y.; Shu, Y.; LaRochelle, J. R.; Steinauer, A.; Thompson, D.; Schepartz, A.; Chen, Z. Y.; Liu, D. R., Discovery and characterization of a peptide that enhances endosomal escape of delivered proteins in vitro and in vivo. J Am Chem Soc 137, 14084-93 (2015); Popp, M. W.; Antos, J. M.; Ploegh, H. L., Site-specific protein labeling via sortase-mediated transpeptidation. Curr Protoc Protein Sci 2009, Chapter 15, Unit 15, 3.

### REFERENCES FOR MATERIALS AND METHODS, GENERAL PROCEDURES, AND SYNTHETIC PROCEDURES

101. T. V. Nguyen et al., Glutamine Triggers Acetylation-Dependent Degradation of Glutamine Synthetase via the Thalidomide Receptor Cereblon. Mol Cell 61, 809-820 (2016).
102. K. R. Ludwig, M. M. Schroll, A. B. Hummon, Comparison of In-Solution, FASP, and S-Trap Based Digestion Methods for Bottom-Up Proteomic Studies. J Proteome Res 17, 2480-2490 (2018).
103. M. HaileMariam et al., S-Trap, an Ultrafast Sample-Preparation Approach for Shotgun Proteomics. J Proteome Res 17, 2917-2924 (2018).
104. W. Huber, A. von Heydebreck, H. Sultmann, A. Poustka, M. Vingron, Variance stabilization applied to microarray data calibration and to the quantification of differential expression. Bioinformatics 18 Suppl 1, S96-104 (2002).
105. I. Chen, B. M. Dorr, D. R. Liu, A general strategy for the evolution of bond-forming enzymes using yeast display. Proc Natl Acad Sci USA 108, 11399-11404 (2011).
106. N. Broguiere, F. A. Formica, G. Barreto, M. Zenobi-Wong, Sortase A as a cross-linking enzyme in tissue engineering. Acta Biomater 77, 182-190 (2018).
107. M. Li et al., Discovery and characterization of a peptide that enhances endosomal escape of delivered proteins in vitro and in vivo. J Am Chem Soc 137, 14084-14093 (2015).
108. H. A. Flaxman, C.-F. Chang, H.-Y. Wu, C. H. Nakamoto, C. M. Woo, A Binding Site Hotspot Map of the FKBP12-Rapamycin-FRB Ternary Complex by Photoaffinity Labeling and Mass Spectrometry-Based Proteomics. J Am Chem Soc 141, 11759-11764 (2019).
109. M. W. Popp, J. M. Antos, H. L. Ploegh, Site-specific protein labeling via sortase-mediated transpeptidation. Curr Protoc Protein Sci Chapter 15, Unit 15 13 (2009). 110. Clontech Laboratories Inc. (2001).
111. ThermoFisher. (https://assets.thermofisher.com/TFS-Assets/MSD/Application-Notes/nanodrop-one-onec-custom-method-hemoglobin-measurements-T144.pdf, 2018).
112. N. Salehi et al., Heme degradation upon production of endogenous hydrogen peroxide via interaction of hem
113. G. E. Winter, D. L. Buckley, J. Paulk, J. M. Roberts, A. Souza, S. Dhe-Paganon, J. E. Bradner, Phthalimide conjugation as a strategy for in vivo target protein degradation. Science 348, 1376-81 (2015).
114. Q. Li, H. Fang, X. Wang, W. Xu, Novel cyclic-imide peptidomimetics as aminopeptidase N inhibitors. Structure-based design, chemistry and activity evaluation. Eur J Med Chem 45, 1618-1626 (2010).
115. J. Yamaguchi, T. Noguchi-Yachide, Y. Sakaguchi, C. Shibata, S. Kanuma, A. Yoshizaki, Y. Takizawa, Y. Hashimoto, Synthesis of New Hydantoins Bearing Glutarimide or Succinimide Moiety and Their Evaluation for Cell Differentiation-inducing and Anti-angiogenic Activities. Heterocycles 91, 764-781 (2015).
116. Y. Shao, M.-Q. Xu, H. Paulus, Protein Splicing: Characterization of the Aminosuccinimide Residue at the Carboxyl Terminus of the Excised Intervening Sequence. Biochemistry 34, 10844-10850 (1995).

### EQUIVALENTS AND SCOPE

In the articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I′): 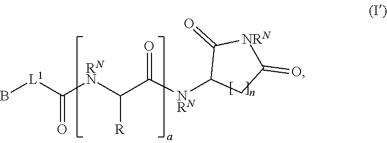
or a pharmaceutically acceptable salt or tautomer thereof, wherein:
B is hydrogen, optionally substituted alkyl, halogen, or a binder of a target, wherein the target is selected from a protein, polypeptide, peptide, carbohydrate, and small molecule;
L¹ is a bond, optionally substituted C₁₋₂₀ alkylene, optionally substituted C₂₋₂₀ alkenylene, optionally substituted C₂₋₂₀ alkynylene, optionally substituted C₁₋₂₀ heteroalkylene, optionally substituted C₂₋₂₀ heteroalkenylene, or optionally substituted C₂₋₂₀ heteroalkynylene, wherein:
optionally one or more backbone carbon atoms of the optionally substituted alkylene, optionally substituted alkenylene, optionally substituted alkynylene, optionally substituted heteroalkylene, optionally substituted heteroalkenylene, and optionally substituted heteroalkynylene are independently replaced with —O—, —S—, —NR^{A}—, —C(═O)—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene; and
optionally one or more backbone heteroatoms of the optionally substituted heteroalkylene, optionally substituted heteroalkenylene, and optionally substituted heteroalkynylene are independently replaced with —O—, —S—, —NR^{A}—, —C(═O)—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene;
R^{N} is hydrogen, optionally substituted alkyl, acyl, or a nitrogen protecting group;
R is hydrogen, optionally substituted alkyl, optionally substituted carbocyclyl, optionally substituted aryl, optionally substituted heteroaryl, or -L¹-B;
optionally where R and R^{N} are joined together to form a optionally substituted 6-membered ring or optionally substituted 5-membered ring;
a is selected from 0, 1, 2, 3, 4, and 5; and
n is selected from 1, 2, and 3;
provided that only one instance of B is a binder of a target.

2. The compound of claim 1, wherein the compound is of Formula (I): 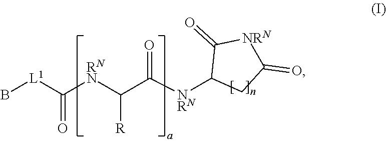
or a pharmaceutically acceptable salt or tautomer thereof, wherein:
B is hydrogen, optionally substituted alkyl, halogen, or a binder of a target, wherein the target is selected from a protein, polypeptide, peptide, carbohydrate, and small molecule;
L¹ is a bond, optionally substituted C₁₋₂₀ alkylene, optionally substituted C₂₋₂₀ alkenylene, optionally substituted C₂₋₂₀ alkynylene, optionally substituted C₁₋₂₀ heteroalkylene, optionally substituted C₂₋₂₀ heteroalkenylene, or optionally substituted C₂₋₂₀ heteroalkynylene, wherein:
optionally one or more backbone carbon atoms of the optionally substituted alkylene, optionally substituted alkenylene, optionally substituted alkynylene, optionally substituted heteroalkylene, optionally substituted heteroalkenylene, and optionally substituted heteroalkynylene are independently replaced with —O—, —S—, —NR^{A}—, —C(═O)—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene; and
optionally one or more backbone heteroatoms of the optionally substituted heteroalkylene, optionally substituted heteroalkenylene, and optionally substituted heteroalkynylene are independently replaced with —O—, —S—, —NR^{A}—, —C(═O)—, —C(═O)NR^{A}—, —NR^{A}C(═O)—, —C(═O)O—, —OC(═O)—, optionally substituted carbocyclylene, optionally substituted heterocyclylene, optionally substituted arylene, or optionally substituted heteroarylene;
R^{N} is hydrogen, optionally substituted alkyl, acyl, or a nitrogen protecting group;
R is hydrogen or optionally substituted alkyl;
optionally where R and R^{N} are joined together to form a 5-membered ring;
a is selected from 0, 1, 2, 3, 4, and 5; and
n is selected from 1, 2, and 3.

3. The compound of claim 1 or 2, wherein the target is a protein, peptide, or polypeptide.

4. The compound of any one of claims 1-3, or a pharmaceutically acceptable salt or tautomer thereof, wherein B is a binder of a target, wherein the target is selected from the group consisting of a bromodomain, a bromodomain-containing protein, a histone methyltransferase, a kinase, a cytosolic signaling protein, a nuclear protein, a histone deacetylase, a lysine methyltransferase, a protein regulating angiogenesis, a protein regulating immune response, an aryl hydrocarbon receptor, a hormone receptor, and a transcription factor.

5. The compound of any one of claims 1-4, or a pharmaceutically acceptable salt or tautomer thereof, wherein R is hydrogen or C₁₋₆ alkyl substituted with —OR^{O}, —O⁻, —SR^{S}, —N(R^{A})₂, —NH₃⁺, —C(═O)N(R^{A})₂, —C(═O)OR^{O}, —C(═O)O⁻, —N(R^{A})C(═NR^{A})N(R^{A})₂, —N(R^{A})C(═N⁺(R^{A})₂)N(R^{A})₂, optionally substituted aryl, optionally substituted heteroaryl, optionally substituted carbocyclyl, or optionally substituted heterocyclyl, wherein:
R^{O} is hydrogen, optionally substituted alkyl, or an oxygen protecting group;
R^{S} is hydrogen, optionally substituted alkyl, or a sulfur protection group; and
R^{A} is hydrogen, optionally substituted alkyl, or a nitrogen protecting group.

6. The compound of any one of claims 1-5, or a pharmaceutically acceptable salt or tautomer thereof, wherein R is an amino acid side chain or derivative thereof.

7. The compound of any one of claims 1-6, or a pharmaceutically acceptable salt or tautomer thereof, wherein R is selected from the group consisting of: 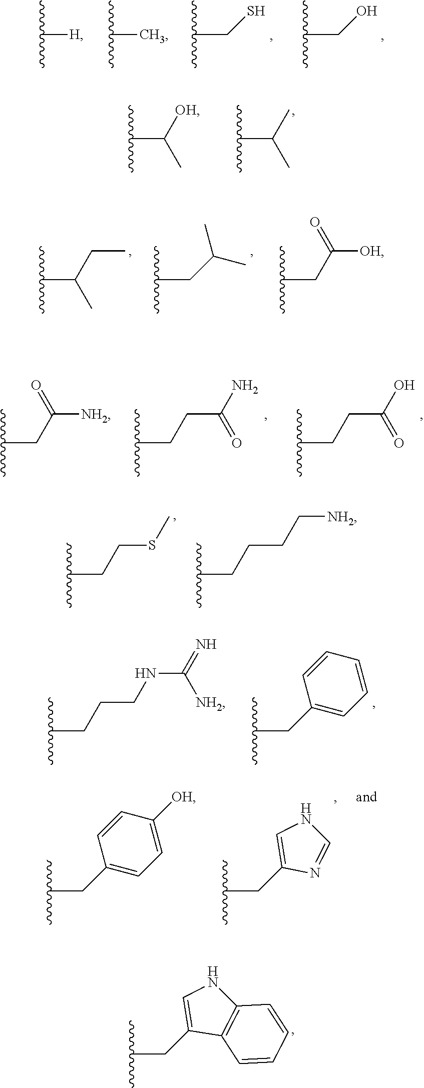
or a pharmaceutically acceptable salt or tautomer thereof.

8. The compound of any one of claims 1-7, or a pharmaceutically acceptable salt or tautomer thereof, wherein R is selected from the group consisting of: 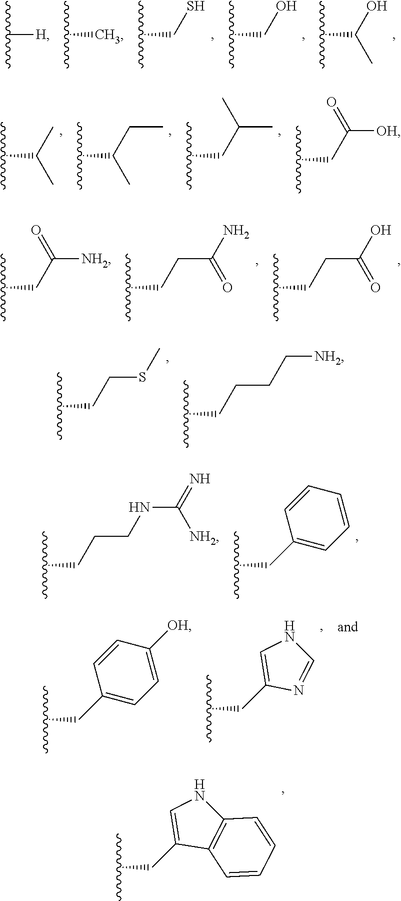
or a pharmaceutically acceptable salt or tautomer thereof.

9. The compound of any one of claims 1-8, wherein R is an amino acid analog.

10. The compound of claim 1, or a pharmaceutically acceptable salt or tautomer thereof, wherein R and R^{N} are joined together to form a 5-membered ring.

11. The compound of any one of claims 1-9, or a pharmaceutically acceptable salt or tautomer thereof, wherein each instance of R^{N} is hydrogen.

12. The compound of any one of claims 1-9 or a pharmaceutically acceptable salt or tautomer thereof, wherein two instances of R^{N} are hydrogen.

13. The compound of any one of claims 1-12, or a pharmaceutically acceptable salt or tautomer thereof, wherein n is 2.

14. The compound of any one of claims 1-12, or a pharmaceutically acceptable salt or tautomer thereof, wherein n is 1.

15. The compound of any one of claims 1-14, or a pharmaceutically acceptable salt or tautomer thereof, wherein a is 1.

16. The compound of any one of claims 1-14, or a pharmaceutically acceptable salt or tautomer thereof, wherein a is 2.

17. The compound of any one of claims 1-12, wherein 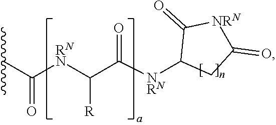
is of the formula:

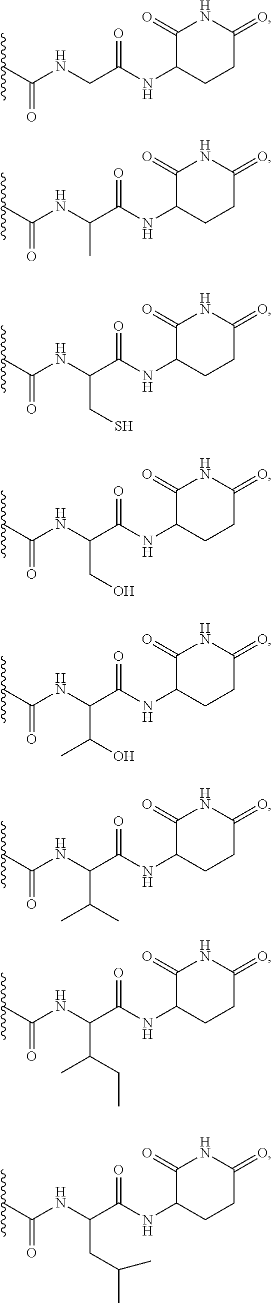

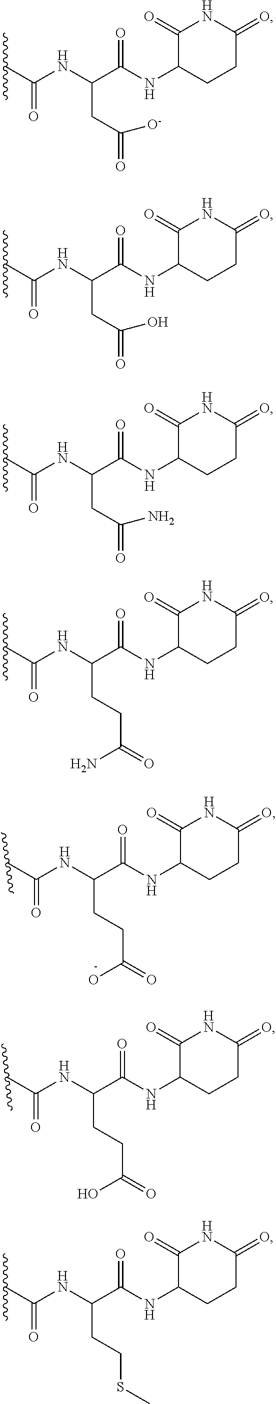

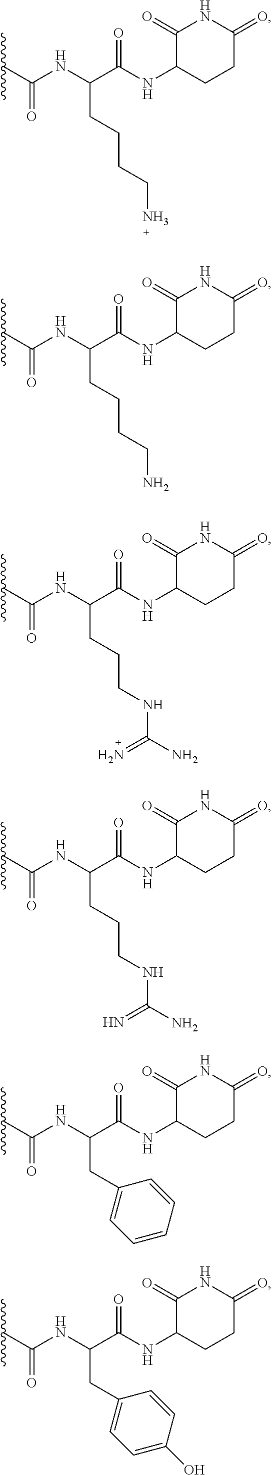

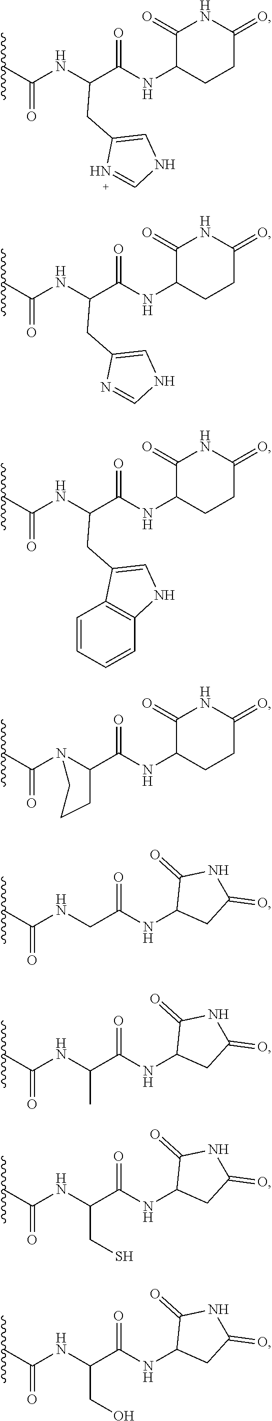

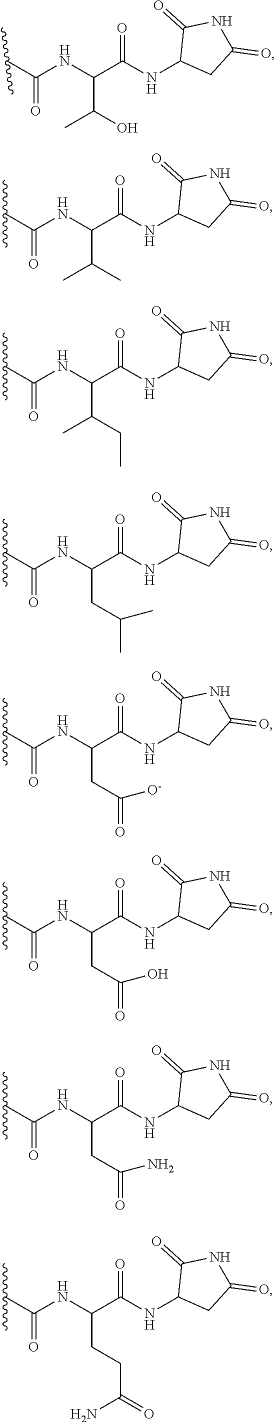

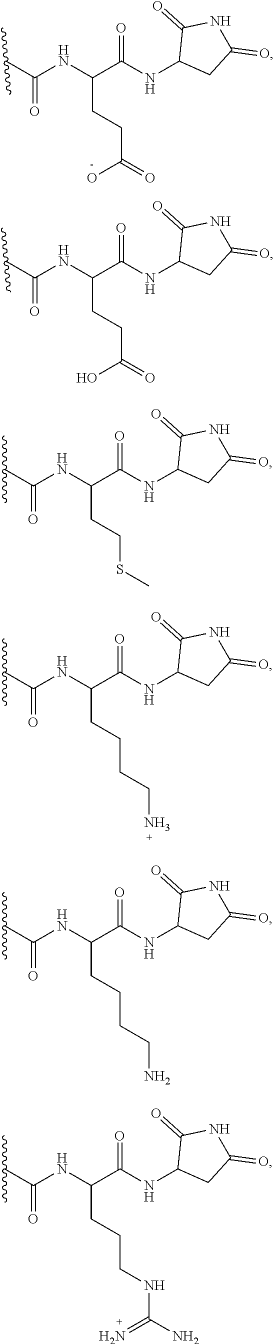

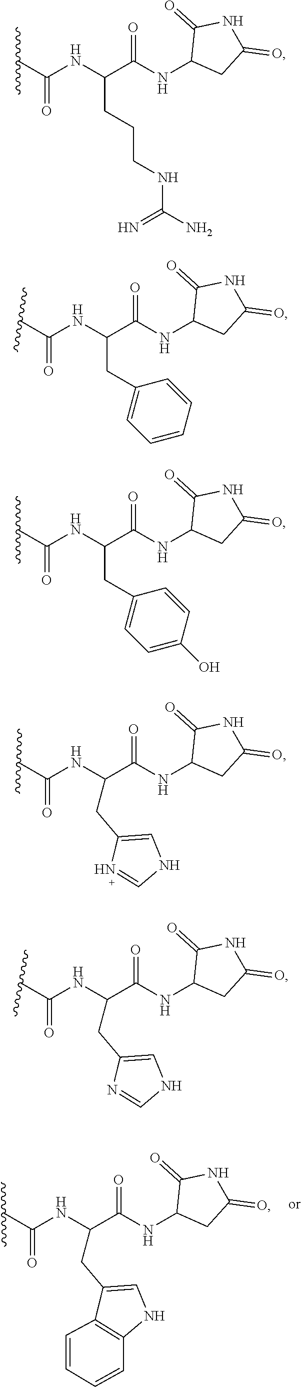

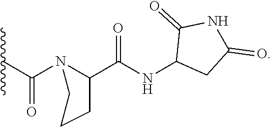 [SMILES: *C(=O)N1CCCC1C(=O)NC1CC(=O)NC1=O]

18. The compound of any one of claims 1-17, or a pharmaceutically acceptable salt or tautomer thereof, wherein L¹ comprises one or more groups independently selected from —O—, —NR^{A}—, —C(═O)NR^{A}—, —NR^{A}C(═O)—, —C(═O)O—, —OC(═O)—, —C(═O)—, 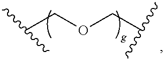
—C═C—, —C≡C—, optionally substituted piperidinylene, optionally substituted piperazinylene, optionally substituted phenylene, optionally substituted triazolylene, and optionally substituted pyrazolylene, wherein: g is an integer from 1 to 10.

19. The compound of any one of claims 1-18, or a pharmaceutically acceptable salt or tautomer thereof, wherein L¹ comprises at least three groups independently selected from —O—, —NR^{A}—, —C(═O)NR^{A}—, —NR^{A}C(═O)—, —C(═O)O—, —OC(═O)—, —C(═O)—, 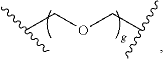
—C═C—, —C≡C—, optionally substituted piperidinylene, optionally substituted piperazinylene, optionally substituted phenylene, optionally substituted triazolylene, and optionally substituted pyrazolylene, wherein g is an integer from 1 to 10.

20. The compound of any one of claims 1-19, or a pharmaceutically acceptable salt or tautomer thereof, wherein L¹ comprises one or more groups independently selected from —O—, —NR^{A}—, —C(═O)NR^{A}—, —NR^{A}C(═O), C(═O)—, —C(═O)O—, and —OC(═O)—.

21. The compound of any one of claims 1-20, or a pharmaceutically acceptable salt or tautomer thereof, wherein L¹ comprises 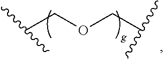
wherein:
g is an integer from 1 to 10.

22. The compound of any one of claims 1-21, or a pharmaceutically acceptable salt or tautomer thereof, wherein L¹ comprises 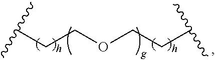
wherein:
g is an integer from 1 to 10; and
each instance of h is independently an integer from 1 to 10.

23. The compound of claim 22, or a pharmaceutically acceptable salt or tautomer thereof, wherein g is 2; and each instance of h is independently 1 or 2.

24. The compound of claim 22, or a pharmaceutically acceptable salt or tautomer thereof, wherein L¹ is

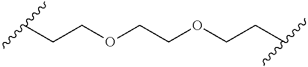 [SMILES: *CCOCCOCC*]

25. The compound of any one of claims 1-17, or a pharmaceutically acceptable salt or tautomer thereof, wherein L¹ is C₄₋₁₆ alkylene.

26. The compound of any one of claims 1-23, or a pharmaceutically acceptable salt or tautomer thereof, wherein L¹ is C₄₋₁₆ alkylene, wherein 1, 2, or 3 backbone carbon atoms of the alkylene are independently replaced with —O—, —NR^{A}—, —C(═O)—, —C(═O)NR^{A}, —NR^{A}C(═O), —C(═O)O—, or —OC(═O)—.

27. The compound of any one of claim 1-23 or 26, or a pharmaceutically acceptable salt or tautomer thereof, wherein L¹ is C₄₋₁₆ alkylene wherein 1 backbone carbon atom of the alkylene is replaced with —C(═O)NR^{A}—, —NR^{A}C(═O)—, —C(═O)—, —C(═O)O—, or —OC(═O)—.

28. The compound of any one of claims 1-23 and 26-27, or a pharmaceutically acceptable salt or tautomer thereof, wherein L¹ is of the formula:

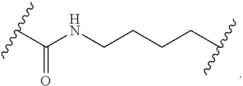 [SMILES: *CCCCNC(*)=O]

29. The compound of any one of claims 1-28, wherein the kinase is a tyrosine kinase, a serine/threonine kinase, a cyclin dependent kinase, or a leucine-rich repeat kinase.

30. The compound of claim 29, wherein the cyclin dependent kinase is cyclin dependent kinase 1 (CDK1), cyclin dependent kinase 2 (CDK2), cyclin dependent kinase 3 (CDK3), cyclin dependent kinase 4 (CDK4), cyclin dependent kinase 5 (CDK5), cyclin dependent kinase 6 (CDK6), cyclin dependent kinase 7 (CDK7), cyclin dependent kinase 8 (CDK8), cyclin dependent kinase 9 (CDK9), cyclin dependent kinase 10 (CDK10), or cyclin dependent kinase 11 (CDK11).

31. The compound of any one of claims 1-28, wherein the cytosolic signaling protein is FKBP12.

32. The compound of any one of claims 1-28, wherein the hormone receptor is an estrogen receptor, an androgen receptor, or a glucocorticoid receptor.

33. The compound of any one of claims 1-28, wherein the transcription factor is SMARCA4, SMARCA2, or TRIM24.

34. The compound of any one of claims 1-33, wherein B is selected from the group consisting of Hsp90 inhibitors, kinase inhibitors, MDM2 inhibitors, compounds targeting bromodomain-containing proteins, BET inhibitors, compounds targeting FKBP, HDAC inhibitors, lysine methyltransferase inhibitors, angiogenesis inhibitors, immunosuppressive compounds, and compounds targeting the aryl hydrocarbon receptor.

35. The compound of any one of claims 1-34, or a pharmaceutically acceptable salt or tautomer thereof, wherein B is selected from the group consisting of JQ1, I-BET-762, OTX-015, I-BET-151, TEN-010, CPI-203, PFI-1, MS436, RVX-297, RVX-208, ABBV-744, CPI-0610, HJB97, rapamycin, FK506, GPI1046, GPI1485, V10367, ElteN378, everolimus, tacrolimus, ridaforolimus, zotarolimus, 3BDO, iRap, AP2167, cRap, pRap, AP23102, AP1510, AP1903, Shield-1, AP20187, ibrutinib, N-piperidine ibrutinib, quizartinib, BI-4464, molibresib, abemaciclib, N-deshydroxyethyl dasatinib, SI-109, navitoclax-piperazine, androstanolone acetate, palbociclib, palbociclib-propargyl, SMARCA-BD, and SLF.

36. The compound of any one of claims 1-35, or a pharmaceutically acceptable salt or tautomer thereof, wherein B is a bromodomain-containing protein 1 (BRD1) binder, bromodomain-containing protein 2 (BRD2) binder, bromodomain-containing protein 3 (BRD3) binder, or bromodomain-containing protein 4 (BRD4) binder.

37. The compound of any one of claims 1-36, or a pharmaceutically acceptable salt or tautomer thereof, wherein B is a bromodomain-containing protein 4 (BRD4) binder.

38. The compound of any one of claims 1-37, or a pharmaceutically acceptable salt or tautomer thereof, wherein B is selected from the group consisting of:

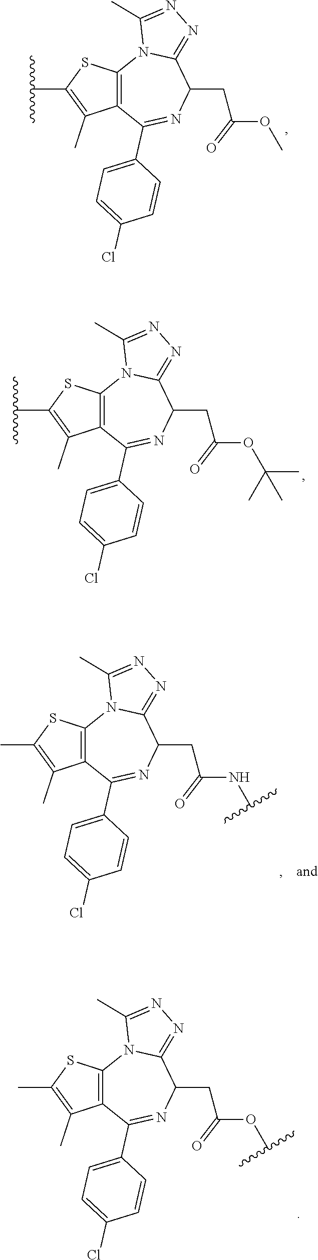

39. The compound of any one of claims 1-38, or a pharmaceutically acceptable salt or tautomer thereof, wherein B is selected from the group consisting of JQ1, I-BET-762, OTX-015, I-BET-151, TEN-010, CPI-203, PFI-1, MS436, RVX-297, RVX-208, ABBV-744, CPI-0610, and HJB97.

40. The compound of any one of claims 1-35, or a pharmaceutically acceptable salt or tautomer thereof, wherein B is a FKBP binder.

41. The compound of claim 40, or a pharmaceutically acceptable salt or tautomer thereof, wherein the FKBP binder is a FKBP12 binder.

42. The compound of any one of claims 1-35 and 40-41, or a pharmaceutically acceptable salt or tautomer thereof, wherein B is

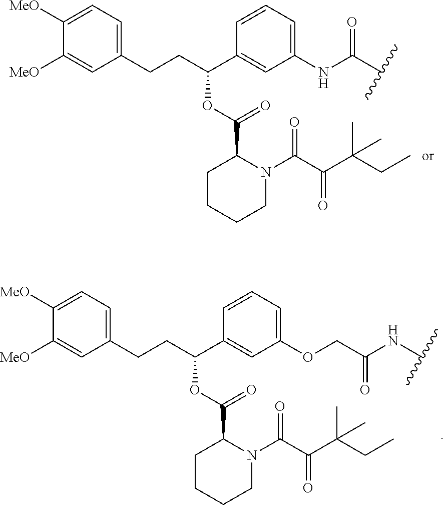 [SMILES: *C(=O)Nc1cccc([C@@H](CCc2ccc(OC)c(OC)c2)OC(=O)[C@@H]2CCCCN2C(=O)C(=O)C(C)(C)CC)c1 ; *NC(=O)COc1cccc([C@@H](CCc2ccc(OC)c(OC)c2)OC(=O)[C@@H]2CCCCN2C(=O)C(=O)C(C)(C)CC)c1]

43. The compound of any one of claims 1-35 and 40-41, or a pharmaceutically acceptable salt or tautomer thereof, wherein B is selected from the group consisting of rapamycin, FK506, GPI1046, GPI1485, V10367, ElteN378, everolimus, tacrolimus, ridaforolimus, zotarolimus, 3BDO, iRap, AP2167, cRap, pRap, AP23102, AP1510, AP1903, Shield-1, and AP20187

44. The compound of any one of claims 1-35, or a pharmaceutically acceptable salt or tautomer thereof, wherein B is a cyclin dependent kinase binder.

45. The compound of any one of claims 1-35 and 44, or a pharmaceutically acceptable salt or tautomer thereof, wherein B is a cyclin dependent kinase 1 (CDK1) binder, cyclin dependent kinase 2 (CDK2) binder, cyclin dependent kinase 3 (CDK3) binder, cyclin dependent kinase 4 (CDK4) binder, cyclin dependent kinase 5 (CDK5) binder, cyclin dependent kinase 6 (CDK6) binder, cyclin dependent kinase 7 (CDK7) binder, cyclin dependent kinase 8 (CDK8) binder, cyclin dependent kinase 9 (CDK9) binder, cyclin dependent kinase 10 (CDK10) binder, or cyclin dependent kinase 11 (CDK11).

46. The compound of any one of claims 1-35 and 44-45, or a pharmaceutically acceptable salt or tautomer thereof, wherein B is a cyclin dependent kinase 4 (CDK4) binder or cyclin dependent kinase 6 (CDK6) binder.

47. The compound of any one of claims 1-35 and 44-46, or a pharmaceutically acceptable salt or tautomer thereof, wherein B is palbociclib.

48. The compound of any one of claims 1-35 and 44-47, or a pharmaceutically acceptable salt or tautomer thereof, wherein B is:

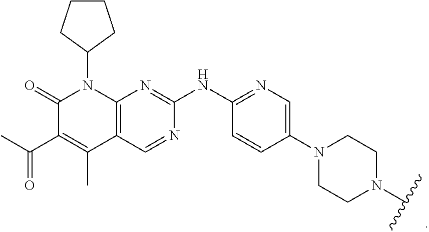 [SMILES: *N1CCN(c2ccc(Nc3ncc4c(C)c(C(C)=O)c(=O)n(C5CCCC5)c4n3)nc2)CC1]

49. The compound of any one of claims 1-3, or a pharmaceutically acceptable salt or tautomer thereof, wherein the compound of Formula (I) is of the formula: 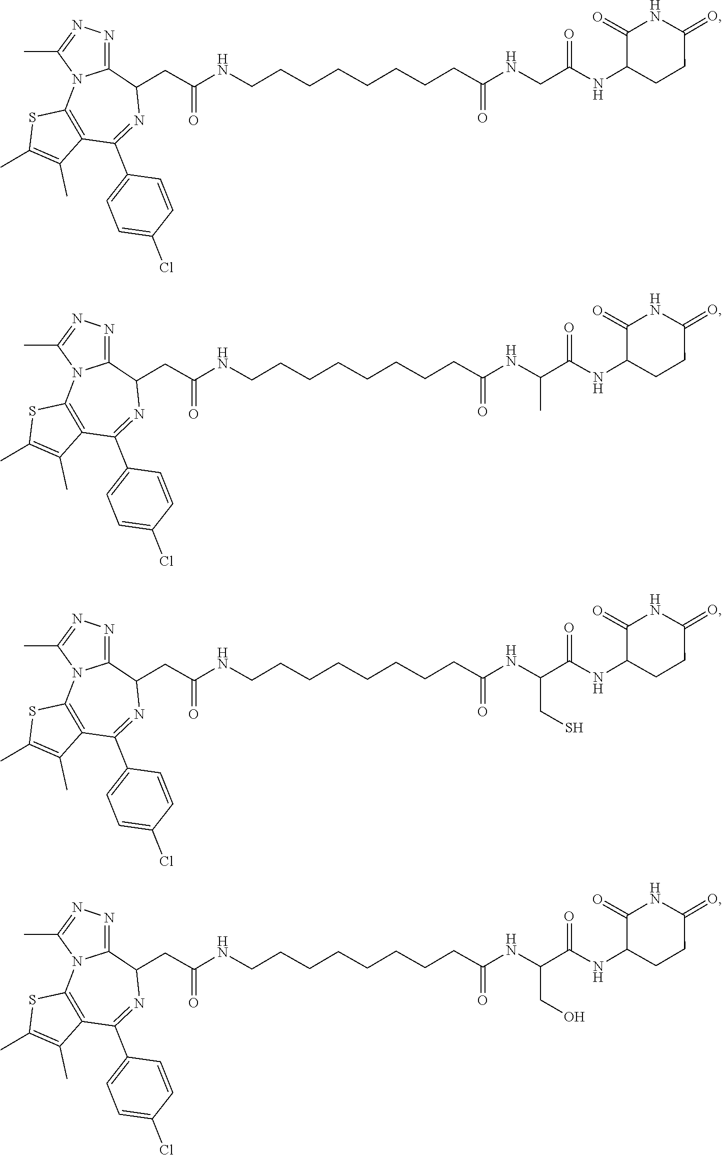 [SMILES: Cc1sc2c(c1C)C(c1ccc(Cl)cc1)=NC(CC(=O)NCCCCCCCCC(=O)NCC(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(C)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CS)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CO)C(=O)NC1CCC(=O)NC1=O)c1nnc(C)n1-2] 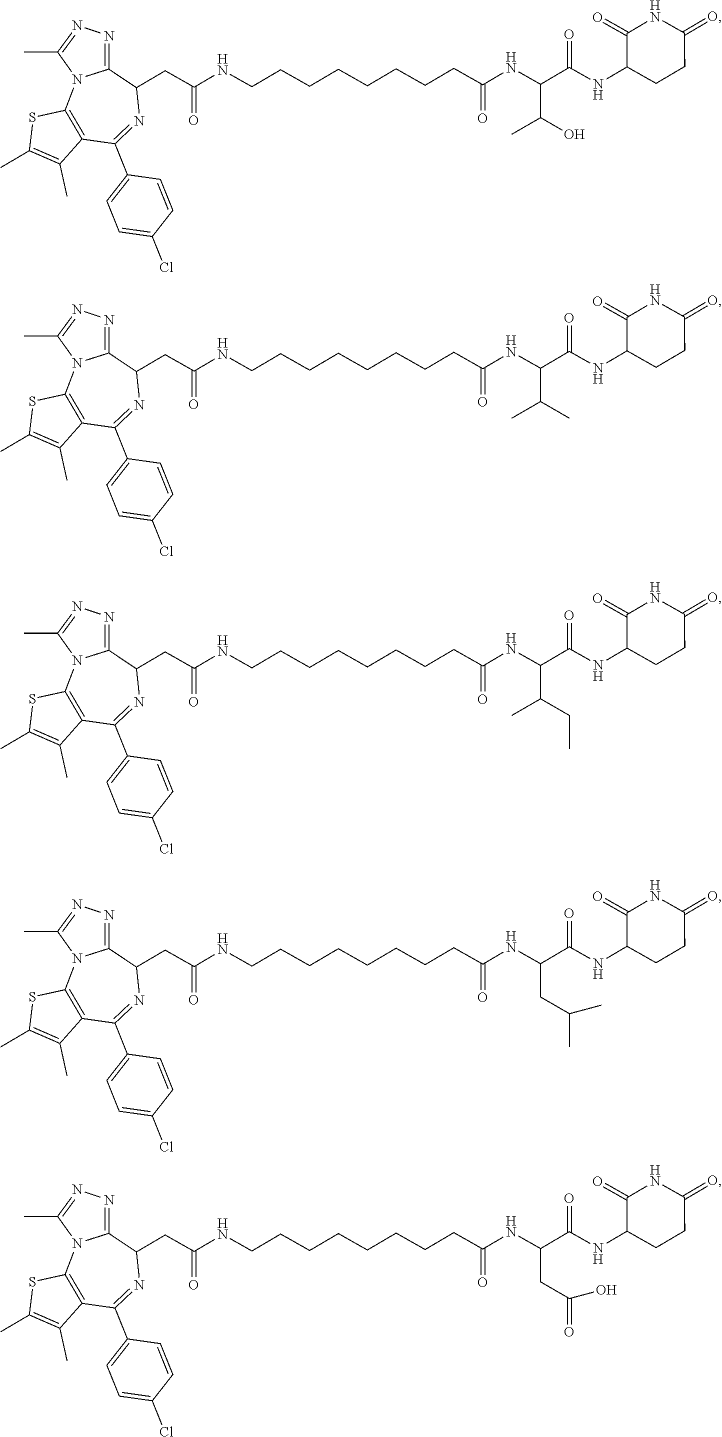 [SMILES: Cc1sc2c(c1C)C(c1ccc(Cl)cc1)=NC(CC(=O)NCCCCCCCCC(=O)NC(C(=O)NC1CCC(=O)NC1=O)C(C)O)c1nnc(C)n1-2 ; Cc1sc2c(c1C)C(c1ccc(Cl)cc1)=NC(CC(=O)NCCCCCCCCC(=O)NC(C(=O)NC1CCC(=O)NC1=O)C(C)C)c1nnc(C)n1-2 ; CCC(C)C(NC(=O)CCCCCCCCNC(=O)CC1N=C(c2ccc(Cl)cc2)c2c(sc(C)c2C)-n2c(C)nnc21)C(=O)NC1CCC(=O)NC1=O ; Cc1sc2c(c1C)C(c1ccc(Cl)cc1)=NC(CC(=O)NCCCCCCCCC(=O)NC(CC(C)C)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CC(=O)O)C(=O)NC1CCC(=O)NC1=O)c1nnc(C)n1-2] 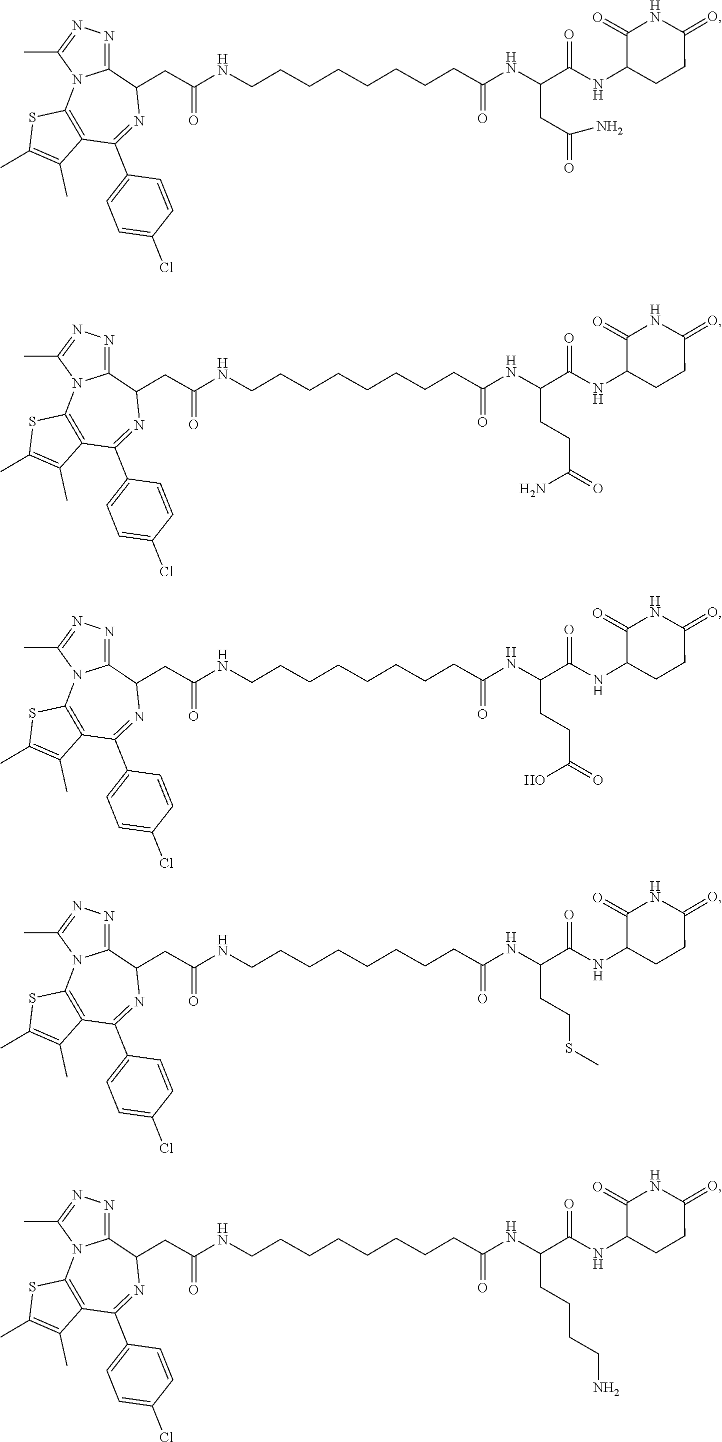 [SMILES: Cc1sc2c(c1C)C(c1ccc(Cl)cc1)=NC(CC(=O)NCCCCCCCCC(=O)NC(CC(N)=O)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CCC(N)=O)C(=O)NC1CCC(=O)NC1=O)c1nnc(C)n1-2 ; Cc1sc2c(c1C)C(c1ccc(Cl)cc1)=NC(CC(=O)NCCCCCCCCC(=O)NC(CCC(=O)O)C(=O)NC1CCC(=O)NC1=O)c1nnc(C)n1-2 ; CSCCC(NC(=O)CCCCCCCCNC(=O)CC1N=C(c2ccc(Cl)cc2)c2c(sc(C)c2C)-n2c(C)nnc21)C(=O)NC1CCC(=O)NC1=O ; Cc1sc2c(c1C)C(c1ccc(Cl)cc1)=NC(CC(=O)NCCCCCCCCC(=O)NC(CCCCN)C(=O)NC1CCC(=O)NC1=O)c1nnc(C)n1-2] 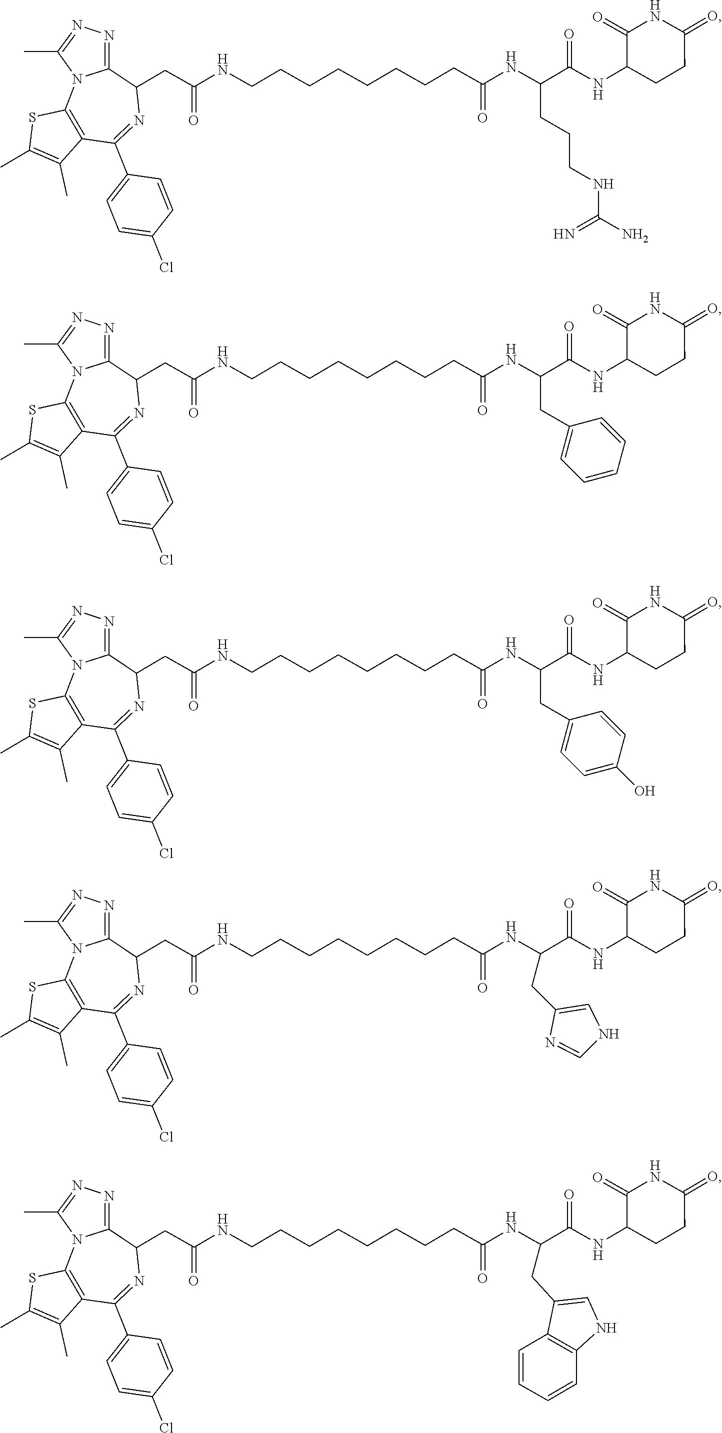 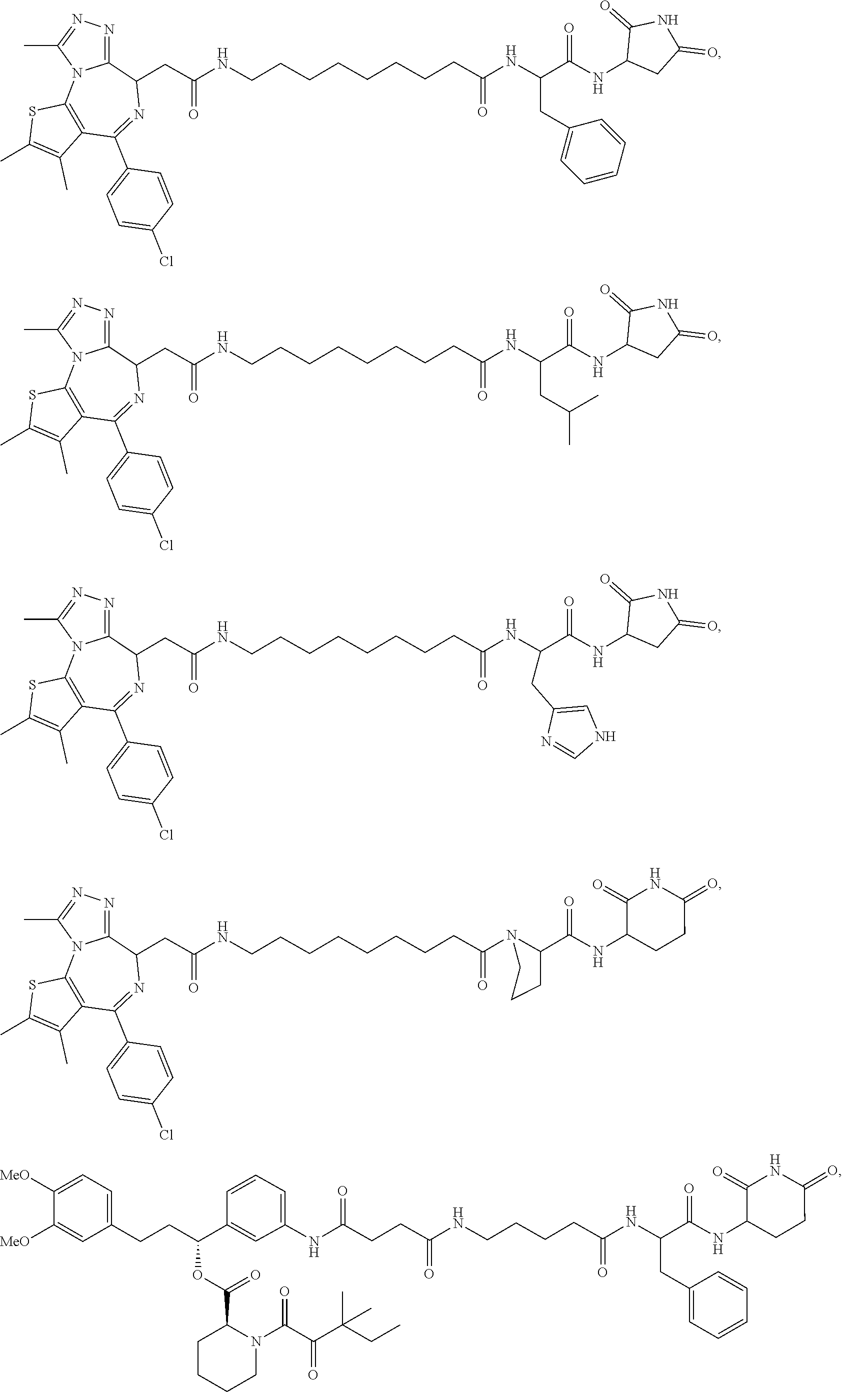 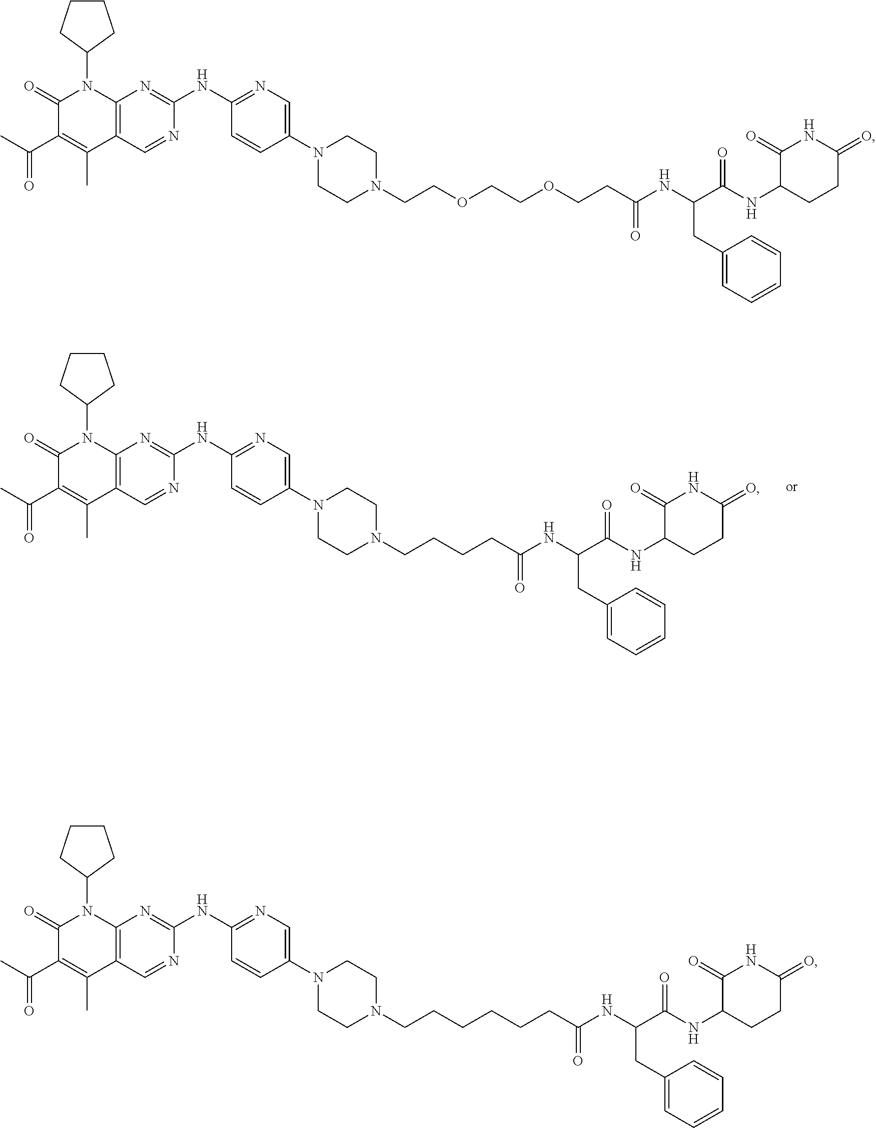 [SMILES: CC(=O)c1c(C)c2cnc(Nc3ccc(N4CCN(CCOCCOCCC(=O)NC(Cc5ccccc5)C(=O)NC5CCC(=O)NC5=O)CC4)cn3)nc2n(C2CCCC2)c1=O ; CC(=O)c1c(C)c2cnc(Nc3ccc(N4CCN(CCCCC(=O)NC(Cc5ccccc5)C(=O)NC5CCC(=O)NC5=O)CC4)cn3)nc2n(C2CCCC2)c1=O ; "or" ; CC(=O)c1c(C)c2cnc(Nc3ccc(N4CCN(CCCCCCC(=O)NC(Cc5ccccc5)C(=O)NC5CCC(=O)NC5=O)CC4)cn3)nc2n(C2CCCC2)c1=O]
or a pharmaceutically acceptable salt or tautomer thereof.

50. A composition comprising a compound of any one of claims 1-49, or a pharmaceutically acceptable salt or tautomer thereof, and optionally a pharmaceutically acceptable excipient.

51. A kit comprising a compound of any one of claims 1-49, or a pharmaceutically acceptable salt or tautomer thereof, or composition of claim 50 and instructions for using the compound or composition.

52. A method of treating or preventing a disease in a subject, the method comprising administering to the subject a therapeutically effective amount of a compound of any one of claims 1-49, or a pharmaceutically acceptable salt or tautomer thereof, or composition of claim 50.

53. A method of treating a disease associated with a target (i.e., the target that B binds to) in a subject in need thereof, the method comprising administering to the subject a therapeutically effective amount of a compound of any one of claims 1-49, or a pharmaceutically acceptable salt or tautomer thereof, or composition of claim 50.

54. A method of treating a disease associated with or mediated by a target (i.e., the target that B binds to) in a subject in need thereof, the method comprising administering to the subject a therapeutically effective amount of a compound of any one of claims 1-49, or a pharmaceutically acceptable salt or tautomer thereof, or composition of claim 50.

55. A method of treating a disease associated with aberrant activity of a target (i.e., the target that B binds to) in a subject in need thereof, the method comprising administering to the subject a therapeutically effective amount of a compound of any one of claims 1-49, or a pharmaceutically acceptable salt or tautomer thereof, or composition of claim 50.

56. A method of treating a disease associated with a bromodomain-containing protein, a bromodomain, a kinase, or a FKBP in a subject in need thereof, the method comprising administering to the subject a therapeutically effective amount of a compound of any one of claims 1-49, or a pharmaceutically acceptable salt or tautomer thereof, or composition of claim 50.

57. A method of treating a disease associated with or mediated by a bromodomain-containing protein, a bromodomain, a kinase, or a FKBP in a subject in need thereof, the method comprising administering to the subject a therapeutically effective amount of a compound of any one of claims 1-49, or a pharmaceutically acceptable salt or tautomer thereof, or composition of claim 50.

58. A method of treating a disease associated with aberrant activity a bromodomain-containing protein, a bromodomain, a kinase, or a FKBP in a subject in need thereof, the method comprising administering to the subject a therapeutically effective amount of a compound of any one of claims 1-49, or a pharmaceutically acceptable salt or tautomer thereof, or composition of claim 50.

59. The method of any one of claims 56-58, wherein the kinase is a cyclin dependent kinase.

60. The method of claim 55 or 58, wherein the aberrant activity is increased activity.

61. The method of any one of claims 52-60, wherein the disease is an inflammatory disease, proliferative disease, autoimmune disease, hematological disease, genetic disease, neurological disease, painful condition, metabolic disorder, infectious disease, cardiovascular disease, cerebrovascular disease, tissue repair disorder, pulmonary disease, dermatological disease, bone disease, or hormonal disease.

62. The method of claim 61, wherein the proliferative disease is cancer.

63. The method of claim 62, wherein the cancer is lung cancer, blood cancer, breast cancer, prostate cancer, pancreatic cancer, colorectal cancer, thyroid cancer, ovarian cancer, neuroblastoma, a carcinoma, a sarcoma, a melanoma, or a tumor.

64. The method of claim 62 or 63, wherein the cancer is nuclear protein of the testis (NUT) midline carcinoma, treatment-refractory acute myeloid leukemia, acute myeloid leukemia (AML), hairy cell leukemia (HCL), acute lymphocytic leukemia (ALL), acute promyelocytic leukemia (APL), chronic lymphocytic leukemia (CLL), chronic myeloid leukemia (CML), myeloproliferative neoplasms (MPN), systemic mastocytosis, plasmacytoma, multiple myeloma, myelodysplastic syndrome, triple negative breast cancer, estrogen receptor-positive breast cancer, small cell lung cancer, non-small cell lung cancer, castration resistant prostate cancer, pancreatic ductal adenocarcinoma, N-Myc Proto-Oncogene Protein (MYCN)-driven solid tumors, Ewing sarcoma, anaplastic thyroid carcinoma (ATC), medulloblastoma, or uveal melanoma.

65. The method of any one of claims 62-64, wherein the cancer is multiple myeloma.

66. The method of claim 64, wherein the myelodysplastic syndrome is del(5q) myelodysplastic syndrome.

67. The method of any one of claims 62-64, wherein the cancer is a hemopoietic cancer.

68. The method of any one of claims 52-61, wherein the disease is an inflammatory disease.

69. The method of claim 68, wherein the inflammatory disease is selected from erythema nodosum leprosum, HIV-associated ulcers, and tuberculous meningitis.

70. The method of any one of claims 52-61, wherein the disease is an autoimmune disease.

71. The method of claim 70, wherein the autoimmune disease is pulmonary fibrosis or systemic lupus erythematosus (SLE).

72. The method of claim 68 or 70, wherein the disease is Crohn's disease, colitis, arthritis, rheumatoid arthritis, or inflammatory bowel disease.

73. The method of any one of claims 52-72, wherein the disease is associated with or mediated by a bromodomain, a bromodomain-containing protein, a histone methyltransferase, a kinase, a cytosolic signaling protein, a nuclear protein, a histone deacetylase, a lysine methyltransferase, a protein regulating angiogenesis, a protein regulating immune response, an aryl hydrocarbon receptor, a hormone receptor, or a transcription factor.

74. The method of any one of claims 52-73, wherein the disease is associated with or mediated by bromodomain, a kinase, or FKBP activity.

75. The method of claim 74, wherein the kinase is a cyclin dependent kinase.

76. A method of modulating the activity of a target (i.e., the target that B binds to) in a subject, the method comprising administering to the subject an effective amount of a compound of any one of claims 1-49, or a pharmaceutically acceptable salt or tautomer thereof, or composition of claim 50.

77. A method of modulating the activity of a target (i.e., the target that B binds to) in a biological sample, the method comprising contacting the biological sample with an effective amount of a compound of any one of claims 1-49, or a pharmaceutically acceptable salt or tautomer thereof, or composition of claim 50.

78. A method of modulating the expression of a gene that is regulated by a target (i.e., the target that B binds to) in a subject, the method comprising administering to the subject an effective amount of a compound of any one of claims 1-49, or a pharmaceutically acceptable salt or tautomer thereof, or composition of claim 50.

79. A method of modulating the activity of a bromodomain-containing protein, a bromodomain, a kinase, or a FKBP in a subject, the method comprising administering to the subject an effective amount of a compound of any one of claims 1-49, or a pharmaceutically acceptable salt or tautomer thereof, or composition of claim 50.

80. A method of modulating the activity of a bromodomain-containing protein, a bromodomain, a kinase, or a FKBP in a biological sample, the method comprising contacting the biological sample with an effective amount of a compound of any one of claims 1-49, or a pharmaceutically acceptable salt or tautomer thereof, or composition of claim 50.

81. A method of modulating the expression of a gene that is regulated by a bromodomain-containing protein, a bromodomain, a kinase, or a FKBP in a subject, the method comprising administering to the subject an effective amount of a compound of any one of claims 1-49, or a pharmaceutically acceptable salt or tautomer thereof, or composition of claim 50.

82. The method of any one of claims 79-81, wherein the kinase is a cyclin dependent kinase.

83. The method of any one of claims 76-81, wherein the method of modulating is a method of inhibiting.

84. A method of inducing the degradation of a protein in a subject, the method comprising administering to the subject an effective amount of a compound of any one of claims 1-49, or a pharmaceutically acceptable salt or tautomer thereof, or composition of claim 50.

85. A method of inducing the degradation of a protein in a cell, tissue, or biological sample, the method comprising administering to the cell, tissue, or biological sample an effective amount of a compound of any one of claims 1-49, or a pharmaceutically acceptable salt or tautomer thereof, or composition of claim 50.

86. The method of any one of claims 53-85, wherein the target, protein, or bromodomain is BRD4.

87. The method of any one of claims 52-86, wherein the method is selective for BRD4.

88. The method of any one of claims 53-85, wherein the target, protein, or FKBP is FKBP12.

89. The method of any one of claims 52-85 and 88, wherein the method inhibits IRF4 expression.

90. The method of any one of claims 53-85, wherein the target, protein, or cyclin-dependent kinase is CDK4 or CDK6.

91. The method of any one of claims 52-85 and 90, wherein the method is selective for CDK4 or CDK6.

92. The method of any one of claims 52-91, wherein the method does not affect off-target transcription factors IKZF1, IKZF3, and SALL4.

93. The method of any one of claims 52-92, wherein the method mitigates off-target interactions compared to an immunomodulatory drug.

94. The method of any one of claims 52-93, wherein the method mitigates off-target degradation compared to an immunomodulatory drug.

95. The method of any one of claims 52-94, wherein the method decreases side effects compared to an immunomodulatory drug.

96. The method of any one of claims 93-95, wherein the immunomodulatory drug is selected from the group consisting of thalidomide, lenalidomide, and pomalidomide.

97. The method of any one of claims 93-96 further comprising administering to the subject an additional therapy.

98. The method of claim 97, wherein the additional therapy is chemotherapy, radioimmunotherapy, surgical therapy, immunotherapy, radiation therapy, or targeted therapy, or any combination thereof.


SEQUENCE LISTING (63 sequences)

SEQ ID NO: 1 - protein, 17 aa, Artificial Sequence
  Synthetic
  MOD_RES (17)..(17): aspartimide
FFESFGDLSTPDAVMGN

SEQ ID NO: 2 - protein, 4 aa, Artificial Sequence
  Synthetic
CKQC

SEQ ID NO: 3 - protein, 9 aa, Artificial Sequence
  Synthetic
HSWFPGYAW

SEQ ID NO: 4 - protein, 4 aa, Artificial Sequence
  Synthetic
CKIC

SEQ ID NO: 5 - protein, 5 aa, Artificial Sequence
  Synthetic
LPETG

SEQ ID NO: 6 - protein, 8 aa, Artificial Sequence
  Synthetic
  MISC_FEATURE (8)..(8): Xaa may be FcQMe, FcQ, FcN, FQ, FN
LPETGGGX

SEQ ID NO: 7 - protein, 8 aa, Artificial Sequence
  Synthetic
  MISC_FEATURE (8)..(8): Xaa may be FcQ, FcN, FQMe
LPETGGGX

SEQ ID NO: 8 - protein, 5 aa, Artificial Sequence
  Synthetic
  MOD_RES (5)..(5): glutarimide
GGGFQ

SEQ ID NO: 9 - protein, 5 aa, Artificial Sequence
  Synthetic
  MOD_RES (5)..(5): aspartimide
GGGFN

SEQ ID NO: 10 - protein, 17 aa, Artificial Sequence
  Synthetic
  MOD_RES (17)..(17): aspartimide
VADALTNAVAHVDDMPN

SEQ ID NO: 11 - protein, 14 aa, Artificial Sequence
  Synthetic
  MOD_RES (14)..(14): aspartimide
VLGAFSDGLAHLDN

SEQ ID NO: 12 - protein, 9 aa, Artificial Sequence
  Synthetic
  MOD_RES (9)..(9): cyclic imide modified alanine
KKVADALTA

SEQ ID NO: 13 - protein, 8 aa, Artificial Sequence
  Synthetic
  MOD_RES (8)..(8): cyclic imide modified alanine
KVADALTA

SEQ ID NO: 14 - protein, 19 aa, Artificial Sequence
  Synthetic
  MOD_RES (19)..(19): cyclic imide modified alanine
KKVADALTNAVAHVDDMPA

SEQ ID NO: 15 - protein, 18 aa, Artificial Sequence
  Synthetic
  MOD_RES (18)..(18): cyclic imide modified alanine
KVADALTNAVAHVDDMPA

SEQ ID NO: 16 - protein, 15 aa, Artificial Sequence
  Synthetic
  MOD_RES (15)..(15): cyclic imide modified valine
KTYFPHFDLSHGSAV

SEQ ID NO: 17 - protein, 18 aa, Artificial Sequence
  Synthetic
  MOD_RES (18)..(18): cyclic imide modified proline
RFFESFGDLSTPDAVMGP

SEQ ID NO: 18 - protein, 8 aa, Artificial Sequence
  Synthetic
  MOD_RES (8)..(8): cyclic imide modified alanine
KEFTPPVA

SEQ ID NO: 19 - protein, 12 aa, Artificial Sequence
  Synthetic
  MOD_RES (12)..(12): cyclic imide modified lysine
KEFTPPVQAAYK

SEQ ID NO: 20 - protein, 15 aa, Artificial Sequence
  Synthetic
  MOD_RES (15)..(15): cyclic imide modified lysine
KVLGAFSDGLAHLDK

SEQ ID NO: 21 - protein, 142 aa, Artificial Sequence
  Synthetic
  MOD_RES (55)..(55): glutarimide
  MOD_RES (69)..(69): aspartimide
  MOD_RES (79)..(79): aspartimide
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 22 - protein, 147 aa, Artificial Sequence
  Synthetic
  MOD_RES (58)..(58): aspartimide
  MOD_RES (81)..(81): aspartimide
  MOD_RES (128)..(128): glutarimide
  MOD_RES (132)..(132): glutarimide
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 23 - protein, 19 aa, Artificial Sequence
  Synthetic
FFESFGDLSTPDAVMGNPK

SEQ ID NO: 24 - protein, 15 aa, Artificial Sequence
  Synthetic
VLGAFSDGLAHLDLK

SEQ ID NO: 25 - protein, 9 aa, Artificial Sequence
  Synthetic
  MOD_RES (8)..(8): aspartimide
KVADALTNA

SEQ ID NO: 26 - protein, 19 aa, Artificial Sequence
  Synthetic
  MOD_RES (18)..(18): aspartimide
KVADALTNAVAHVDDMPNA

SEQ ID NO: 27 - protein, 16 aa, Artificial Sequence
  Synthetic
  MOD_RES (15)..(15): glutarimide
KTYFPHFDLSHGSAQV

SEQ ID NO: 28 - protein, 19 aa, Artificial Sequence
  Synthetic
  MOD_RES (18)..(18): aspartimide
RFFESFGDLSTPDAVMGNP

SEQ ID NO: 29 - protein, 16 aa, Artificial Sequence
  Synthetic
  MOD_RES (15)..(15): aspartimide
KVLGAFSDGLAHLDNL

SEQ ID NO: 30 - protein, 9 aa, Artificial Sequence
  Synthetic
  MOD_RES (8)..(8): glutarimide
KEFTPPVQA

SEQ ID NO: 31 - protein, 13 aa, Artificial Sequence
  Synthetic
  MOD_RES (12)..(12): glutarimide
KEFTPPVQAAYQK

SEQ ID NO: 32 - protein, 18 aa, Artificial Sequence
  Synthetic
  MOD_RES (17)..(17): aspartimide
RVAPEEHPVLLTEAPLNP

SEQ ID NO: 33 - protein, 5 aa, Artificial Sequence
  Synthetic
  MOD_RES (5)..(5): glutarimide
GGGLQ

SEQ ID NO: 34 - protein, 23 aa, Artificial Sequence
  Synthetic
FFESFFESFGDLSTPDAVMGNPK

SEQ ID NO: 35 - protein, 21 aa, Artificial Sequence
  Synthetic
  MOD_RES (21)..(21): aspartimide
FFESFFESFGDLSTPDAVMGN

SEQ ID NO: 36 - protein, 21 aa, Artificial Sequence
  Synthetic
FFESFFESFGDLSTPDAVMGN

SEQ ID NO: 37 - protein, 18 aa, Artificial Sequence
  Synthetic
VAPEEHPVLLTEAPLNPK

SEQ ID NO: 38 - protein, 16 aa, Artificial Sequence
  Synthetic
  MOD_RES (16)..(16): aspartimide
VAPEEHPVLLTEAPLN

SEQ ID NO: 39 - protein, 16 aa, Artificial Sequence
  Synthetic
VAPEEHPVLLTEAPLN

SEQ ID NO: 40 - protein, 29 aa, Artificial Sequence
  Synthetic
VADALTNAVAHVDDMPNALSALSDLHAHK

SEQ ID NO: 41 - protein, 17 aa, Artificial Sequence
  Synthetic
VADALTNAVAHVDDMPN

SEQ ID NO: 42 - protein, 7 aa, Artificial Sequence
  Synthetic
  MOD_RES (7)..(7): aspartimide
VADALTN

SEQ ID NO: 43 - protein, 7 aa, Artificial Sequence
  Synthetic
VADALTN

SEQ ID NO: 44 - protein, 19 aa, Artificial Sequence
  Synthetic
FFESFGDLSTPDAVMGNPK

SEQ ID NO: 45 - DNA, 18 bp, Artificial Sequence
  Synthetic
caccaccaccaccaccac

SEQ ID NO: 46 - DNA, 47 bp, Artificial Sequence
  Synthetic
accggtttccgggaggctaccaccggtcttgtacagctcgtccatgc

SEQ ID NO: 47 - DNA, 29 bp, Artificial Sequence
  Synthetic
ggcggcagcggagtgcaggtggaaaccat

SEQ ID NO: 48 - DNA, 20 bp, Artificial Sequence
  Synthetic
ttttggaggatggtcgccac

SEQ ID NO: 49 - DNA, 49 bp, Artificial Sequence
  Synthetic
ggccaccatcaccatcaccactaaactagaattcatcgtgactgactga

SEQ ID NO: 50 - DNA, 50 bp, Artificial Sequence
  Synthetic
gcccgtctcaggtaacgagcctccagtcagatcctcttctgagatgagtt

SEQ ID NO: 51 - DNA, 21 bp, Artificial Sequence
  Synthetic
catgtacgttgctatccaggc

SEQ ID NO: 52 - DNA, 21 bp, Artificial Sequence
  Synthetic
ctccttaatgtcacgcacgat

SEQ ID NO: 53 - DNA, 21 bp, Artificial Sequence
  Synthetic
aatggcacaaacgctggaaaa

SEQ ID NO: 54 - DNA, 19 bp, Artificial Sequence
  Synthetic
cactggtaacactgccctg

SEQ ID NO: 55 - DNA, 21 bp, Artificial Sequence
  Synthetic
gagctacccacagaagaaacc

SEQ ID NO: 56 - DNA, 22 bp, Artificial Sequence
  Synthetic
gagtcgatgcttgagttgtgtt

SEQ ID NO: 57 - DNA, 21 bp, Artificial Sequence
  Synthetic
ttccgtgatccttttgagtgc

SEQ ID NO: 58 - DNA, 19 bp, Artificial Sequence
  Synthetic
ctcgcgttatgtgcgacga

SEQ ID NO: 59 - protein, 9 aa, Artificial Sequence
  Synthetic
TGGSLPETG

SEQ ID NO: 60 - protein, 5 aa, Artificial Sequence
  Synthetic
  MOD_RES (5)..(5): methylated glutarimide
GGGFQ

SEQ ID NO: 61 - protein, 5 aa, Artificial Sequence
  Synthetic
  MOD_RES (5)..(5): glutarimide
GGGPQ

SEQ ID NO: 62 - protein, 5 aa, Artificial Sequence
  Synthetic
  MOD_RES (5)..(5): aspartimide
GGGLN

SEQ ID NO: 63 - protein, 5 aa, Artificial Sequence
  Synthetic
  MOD_RES (5)..(5): any cyclized amino acid
  MOD_RES (5)..(5): Xaa is any cyclized amino acid
GGGFX